(19) United States
(12) Patent Application Publication
(10) Pub. No.: US 2009/0208531 A1
(43) Pub. Date: Aug. 20, 2009

(54) ANTIVIRAL AGENTS AND VACCINES AGAINST INFLUENZA

(75) Inventors: Gary J. Nabel, Washington, DC (US); Wing-pui Kong, Germantown, MD (US); Zhi-yong Yang, Potomac, MD (US)

Correspondence Address: KNOBBE, MARTENS, OLSON & BEAR, LLP, 2040 MAIN STREET, FOURTEENTH FLOOR, IRVINE, CA, 92614, US

(73) Assignee: National Institutes of Health Office of Technology, Rockville, MD (US)

(21) Appl. No.: 12/279,332

(22) PCT Filed: Feb. 16, 2007
(86) PCT No.: PCT/US07/04506

§ 371 Date: Feb. 17, 2009

Related U.S. Application Data
(60) Provisional application No. 60/774,923, filed on Feb. 16, 2006.

(51) Int. Cl. A61K 39/145; C12N 15/11; A61K 31/7088; C12N 15/00

(52) U.S. Cl. 424/209.1; 536/23.1; 435/320.1; 514/44 R; 435/5

(57) ABSTRACT

These vaccines target H5N1, H1, H3 and other subtypes of influenza and are designed to elicit neutralizing antibodies, as well as cellular immunity. The DNA vaccines express hemagglutinin (HA) or nucleoprotein (NP) proteins from influenza which are codon optimized and/or contain modifications to protease cleavage sites of HA which affect the normal function of the protein. Adenoviral constructs expressing the same inserts have been engineered for prime boost strategies. Protein-based vaccines based on protein production from insect or mammalian cells using foldon trimerization stabilization domains with or without cleavage sites to assist in purification of such proteins have been developed. Another embodiment of this invention is the work with HA pseudotyped lentiviral vectors which would be used to screen for neutralizing antibodies in patients and to screen for diagnostic and therapeutic antivirals such as monoclonal antibodies.

### RELATED APPLICATIONS

This application claims the benefit of U.S. Provisional Application No. 60/774,923 filed Feb. 16, 2006 which is hereby incorporated by reference in its entirety.

### FIELD OF THE INVENTION

The present invention relates to the field of molecular biology. The present invention discloses influenza virus proteins, related nucleotide sequences, and usage for immunization by gene-based vaccines and recombinant proteins.

### DESCRIPTION OF THE RELATED ART

The significant public health impact of Influenza A and B virus infections is compounded by the threat of emerging virus strains. Concerns exist that avian influenza virus (H5N1), endemic in poultry in Southeast Asia, may trigger a pandemic in humans should the virus evolve to spread from human-to-human. The currently licensed influenza vaccines include inactivated influenza vaccines, propagated in embryonated chicken eggs (i.e., Fluzone®, Sanofi Pasteur, Inc.; Fluvirin®), Chiron Corporation; Flaurix™, GlaxoSmithKline, Inc.), and a cold-adapted, live attenuated influenza vaccine delivered intranasally (Flumist®, Medimmune Vaccines, Inc.). While highly efficacious, these vaccines depend upon labor-intensive methods and limited manufacturing capacity. Sanofi Pasteur, Inc. and Chiron Corporation are both producing inactivated vaccines for H5N1 avian influenza. The Sanofi Pasteur, Inc. product has proven to be well-tolerated in 300 volunteers (Sanofi_Pasteur, on the world-wide web at sanofipasteur.com/sanofipasteur/front/templates/vaccinations-travel-health-vaccine-aventis-pasteurjsp?&lang=EN&codeRubrique=13&codePage=CP_{—}15_{—}12_{—}2005, (cited Dec. 15, 2005)). However, there is serious concern that the currently available production methodology cannot meet world-wide public health needs.

Several new technologies have undergone evaluation in hundreds of research subjects in clinical studies, including protein subunit vaccines directed against Influenza A and avian influenza H5N1 strains (Protein Sciences Corporation, on the world-wide-web at proteinsciences.com/aboutus/pdf/PhaseII-IIIresults-June2005-2.pdf., cited Jun. 14, 2005), virosomes or lipid antigen-presenting systems (Solvay Pharmaceuticals) (de Bruijn, I. A. et al. 2005 Vaccine 23 (Suppl I):S39-49), adenoviral vectored vaccines (Vaxin (Van Kampen, K. R. et al. 2005 Vaccine 23:1029-1036)) and an epidermal DNA vaccine, coated onto gold beads and delivered by the PowderJect device (Drape, R. J. et al. 2005 Vaccine 24:4475-4481 2005). Other technology, including recombinant particulate vaccines with influenza proteins assembled into virus-like particles, are in preclinical stages of evaluation (Girard, M. P. et al. 2005 Vaccine 23:5708-5724).

A report of a February 2004 World Health Organization meeting underscored the need for new broad-spectrum influenza vaccines capable of inducing long-lasting immune responses (Cassetti, M. C. et al 2005 Vaccine 23:1529-1533). The meeting participants recommended that plasmid DNA-based technology, having demonstrated preclinical efficacy and fast and relatively easy manufacturing processes, should be assessed as an alternative to conventional influenza strategies (Cassetti, M. C. et al. 2005 Vaccine 23:1529-1533). The goal would be to develop a broader more universal vaccine that would protect against multiple influenza strains.

### SUMMARY OF THE INVENTION

This invention describes the development of plasmid DNA vaccines and plasmid DNA prime/protein boost strategies for prevention of influenza.

These vaccines target H5N1, H1, H3 and other subtypes of influenza and are designed to elicit neutralizing antibodies, as well as cellular immunity. The DNA vaccines express hemagglutinin (HA) or nucleoprotein (NP) proteins from influenza which are codon optimized and/or contain modifications to protease cleavage sites of HA which affect the normal function of the protein. They have been constructed in a different CMV/R or CMV/R 8 κB expression backbone. Adenoviral constructs expressing the same inserts have been engineered for prime boost strategies.

Protein-based vaccines based on protein production from insect or mammalian cells using foldon trimerization stabilization domains with or without cleavage sites to assist in purification of such proteins have been developed.

This invention provides a vaccine strategy for controlling influenza epidemics, including avian flu, should it cross over to humans, the 1918 strain of flu, and seasonal flu strains. In addition, the invention is designed to lead to a combination vaccine to provide a broadly protective vaccine.

Another embodiment of this invention is the work with HA pseudotyped lentiviral vectors which would be used to screen for neutralizing antibodies in patients and to screen for diagnostic and therapeutic antivirals such as monoclonal antibodies.

### BRIEF DESCRIPTION OF THE DRAWINGS

FIG. 1. Schematic diagram and nucleic acid sequence of VRC 9123.

FIG. 2. Schematic diagram and nucleic acid sequence of VRC 7702.

FIG. 3. Schematic diagram and nucleic acid sequence of VRC 7703.

FIG. 4. Schematic diagram and nucleic acid sequence of VRC 7704.

FIG. 5. Schematic diagram and nucleic acid sequence of VRC 7705.

FIG. 6. Schematic diagram and nucleic acid sequence of VRC 7706.

FIG. 7. Schematic diagram and nucleic acid sequence of VRC 7707.

FIG. 8. Schematic diagram and nucleic acid sequence of VRC 7708.

FIG. 9. Schematic diagram and nucleic acid sequence of VRC 7712.

FIG. 10. Schematic diagram and nucleic acid sequence of VRC 7713.

FIG. 11. Schematic diagram and nucleic acid sequence of VRC 7714.

FIG. 12. Schematic diagram and nucleic acid sequence of VRC 7715.

FIG. 13. Schematic diagram and nucleic acid sequence of VRC 7716.

FIG. 14. Schematic diagram and nucleic acid sequence of VRC 7717.

FIG. 15. Schematic diagram and nucleic acid sequence of VRC 7718.

FIG. 16. Schematic diagram and nucleic acid sequence of VRC 7719.

FIG. 17. Schematic diagram and nucleic acid sequence of 53349.

FIG. 18. Schematic diagram and nucleic acid sequence of 53350.

FIG. 19. Schematic diagram and nucleic acid sequence of 53352.

FIG. 20. Schematic diagram and nucleic acid sequence of 53353.

FIG. 21. Schematic diagram and nucleic acid sequence of 53355.

FIG. 22. Schematic diagram and nucleic acid sequence of 53356.

FIG. 23. Schematic diagram and nucleic acid sequence of 53358.

FIG. 24. Schematic diagram and nucleic acid sequence of 53359.

FIG. 25. Schematic diagram and nucleic acid sequence of 53361.

FIG. 26. Schematic diagram and nucleic acid sequence of 53362.

FIG. 27. Schematic diagram and nucleic acid sequence of 53364.

FIG. 28. Schematic diagram and nucleic acid sequence of 53365.

FIG. 29. Schematic diagram and nucleic acid sequence of 53367.

FIG. 30. Schematic diagram and nucleic acid sequence of 53320.

FIG. 31. Schematic diagram and nucleic acid sequence of 53322.

FIG. 32. Schematic diagram and nucleic acid sequence of 53325.

FIG. 33. Schematic diagram and nucleic acid sequence of 53326.

FIG. 34. Schematic diagram and nucleic acid sequence of 53328.

FIG. 35. Schematic diagram and nucleic acid sequence of 53331.

FIG. 36. Schematic diagram and nucleic acid sequence of 53332.

FIG. 37. Schematic diagram and nucleic acid sequence of 53334.

FIG. 38. Schematic diagram and nucleic acid sequence of 53335.

FIG. 39. Schematic diagram and nucleic acid sequence of 53336.

FIG. 40. Schematic diagram and nucleic acid sequence of 53337.

FIG. 41. Schematic diagram and nucleic acid sequence of 53338.

FIG. 42. Schematic diagram and nucleic acid sequence of 53340.

FIG. 43. Schematic diagram and nucleic acid sequence of 53955.

FIG. 44. Schematic diagram and nucleic acid sequence of 53367.

FIG. 45. Schematic diagram and nucleic acid sequence of 53504.

FIG. 46. Schematic diagram and nucleic acid sequence of 53510.

FIG. 47. Schematic diagram and nucleic acid sequence of 53515.

FIG. 48. Schematic diagram and nucleic acid sequence of 54567.

FIG. 49. Schematic diagram and nucleic acid sequence of 54568.

FIG. 50. Schematic diagram and nucleic acid sequence of 54569.

FIG. 51. Schematic diagram and nucleic acid sequence of 54570.

FIG. 52. Schematic diagram and nucleic acid sequence of 53956.

FIG. 53. Schematic diagram and nucleic acid sequence of 53957.

FIG. 54. Schematic diagram and nucleic acid sequence of 53967.

FIG. 55. Schematic diagram and nucleic acid sequence of 53329.

FIG. 56. Schematic diagram and nucleic acid sequence of 53330.

FIG. 57. Schematic diagram and nucleic acid sequence of 53331.

FIG. 58. Schematic diagram and nucleic acid sequence of 53503.

FIG. 59. Schematic diagram and nucleic acid sequence of 51490.

FIG. 60. Schematic diagram and nucleic acid sequence of 51491.

FIG. 61. Schematic diagram and nucleic acid sequence of 51492.

FIG. 62. Schematic diagram and nucleic acid sequence of 51493.

FIG. 63. Schematic diagram and nucleic acid sequence of 51494.

FIG. 64. Schematic diagram and nucleic acid sequence of 51495.

FIG. 65. Schematic diagram and nucleic acid sequence of 51497.

FIG. 66. Schematic diagram and nucleic acid sequence of 51498.

FIG. 67. Schematic diagram and nucleic acid sequence of 51499.

FIG. 68. Schematic diagram and nucleic acid sequence of 51804.

FIG. 69. Schematic diagram and nucleic acid sequence of 51805.

FIG. 70. Schematic diagram and nucleic acid sequence of 51803.

FIG. 71. Schematic diagram and nucleic acid sequence of 53335.

FIG. 72. Schematic diagram and nucleic acid sequence of 53336.

FIG. 73. Schematic diagram and nucleic acid sequence of 53337.

FIG. 74. Schematic diagram and nucleic acid sequence of 53505.

FIG. 75. Schematic diagram and nucleic acid sequence of 53508.

FIG. 76. Schematic diagram and nucleic acid sequence of 53323.

FIG. 77. Schematic diagram and nucleic acid sequence of 53344.

FIG. 78. Schematic diagram and nucleic acid sequence of 53346.

FIG. 79. Schematic diagram and nucleic acid sequence of 53353.

FIG. 80. Schematic diagram and nucleic acid sequence of 53355.

FIG. 81. Schematic diagram and nucleic acid sequence of 53356.

FIG. 82. Schematic diagram and nucleic acid sequence of 53358.

FIG. 83. Schematic diagram and nucleic acid sequence of 53501.

FIG. 84. Schematic diagram and nucleic acid sequence of 53502.

FIG. 85. Schematic diagram and nucleic acid sequence of 53506.

FIG. 86. Schematic diagram and nucleic acid sequence of 53508.

FIG. 87. Schematic diagram and nucleic acid sequence of 53511.

FIG. 88. Schematic diagram and nucleic acid sequence of 53512.

FIG. 89. Schematic diagram and nucleic acid sequence of 54671.

FIG. 90. Schematic diagram and nucleic acid sequence of 54672.

FIG. 91. Schematic diagram and nucleic acid sequence of 54673.

FIG. 92. Schematic diagram and nucleic acid sequence of 54675.

FIG. 93. Schematic diagram and nucleic acid sequence of 54678.

FIG. 94. Schematic diagram and nucleic acid sequence of 54679.

FIG. 95. Schematic diagram and nucleic acid sequence of 53500.

FIG. 96. Schematic diagram and nucleic acid sequence of 53509.

FIG. 97. Schematic diagram and nucleic acid sequence of 53513.

FIG. 98. Schematic diagram and nucleic acid sequence of 53514.

FIG. 99. Schematic diagram and nucleic acid sequence of 56382.

FIG. 100. Schematic diagram and nucleic acid sequence of 54580.

FIG. 101. Schematic diagram and nucleic acid sequence of 54581.

FIG. 102. Schematic diagram and nucleic acid sequence of 54582.

FIG. 103. Schematic diagram and nucleic acid sequence of 54583.

FIG. 104. Schematic diagram and nucleic acid sequence of 54680.

FIG. 105. Schematic diagram and nucleic acid sequence of 54681.

FIG. 106. Schematic diagram and nucleic acid sequence of 54682.

FIG. 107. Schematic diagram and nucleic acid sequence of 54563.

FIG. 108. Schematic diagram and nucleic acid sequence of 54564.

FIG. 109. Schematic diagram and nucleic acid sequence of 54565.

FIG. 110. Schematic diagram and nucleic acid sequence of 54566.

FIG. 111. Schematic diagram and nucleic acid sequence of 54670.

FIG. 112. Schematic diagram and nucleic acid sequence of 54676.

FIG. 113. Schematic diagram and nucleic acid sequence of 54677.

FIG. 114. Schematic diagram and nucleic acid sequence of 53957.

FIG. 115. Schematic diagram and nucleic acid sequence of 54510.

FIG. 116. Schematic diagram and nucleic acid sequence of 54671.

FIG. 117. Schematic diagram and nucleic acid sequence of 54672.

FIG. 118. Schematic diagram and nucleic acid sequence of 54675.

FIG. 119. Schematic diagram and nucleic acid sequence of 54678.

FIG. 120. Schematic diagram and nucleic acid sequence of 54679.

FIG. 121. Schematic diagram and nucleic acid sequence of 56383.

FIG. 122. Schematic diagram and nucleic acid sequence of 56384.

FIG. 123. Schematic diagram and nucleic acid sequence of 56478.

FIG. 124. Schematic diagram and nucleic acid sequence of 56479.

FIG. 125. Schematic diagram and nucleic acid sequence of VRC 7700.

FIG. 126. Schematic diagram and nucleic acid sequence of VRC 7710.

FIG. 127. Schematic diagram and nucleic acid sequence of VRC 7720.

FIG. 128. Schematic diagram and nucleic acid sequence of VRC 7730.

FIG. 129. Schematic diagram and nucleic acid sequence of VRC 7731.

FIG. 130. Schematic diagram and nucleic acid sequence of VRC 7732.

FIG. 131. Schematic diagram and nucleic acid sequence of VRC 7733.

FIG. 132. Schematic diagram and nucleic acid sequence of VRC 7734.

FIG. 133. Schematic diagram and nucleic acid sequence of VRC 7735.

FIG. 134. Schematic diagram and nucleic acid sequence of VRC 7742.

FIG. 135. Schematic diagram and nucleic acid sequence of VRC 7721.

FIG. 136. Schematic diagram and nucleic acid sequence of VRC 7743.

FIG. 137. Schematic diagram and nucleic acid sequence of VRC 7744.

FIG. 138. Schematic diagram and nucleic acid sequence of VRC 7745.

FIG. 139. Schematic diagram and nucleic acid sequence of VRC 7746.

FIG. 140. Schematic diagram and nucleic acid sequence of VRC 7747.

FIG. 141. Schematic diagram and nucleic acid sequence of VRC 7748.

FIG. 142. Schematic diagram and nucleic acid sequence of VRC 7749.

FIG. 143. Schematic diagram and nucleic acid sequence of VRC 7751.

FIG. 144. Schematic diagram and nucleic acid sequence of VRC 7752.

FIG. 145. Schematic diagram and nucleic acid sequence of VRC 7753.

FIG. 146. Schematic diagram and nucleic acid sequence of VRC 7754.

FIG. 147. Schematic diagram and nucleic acid sequence of VRC 7755.

FIG. 148. Schematic diagram and nucleic acid sequence of VRC 7757.

FIG. 149. Schematic diagram and nucleic acid sequence of VRC 7758.

FIG. 150. Schematic diagram and nucleic acid sequence of VRC 7759.

FIG. 151. A schematic diagram of the structure of the influenza A virus particle.

FIG. 152. Diagram of influenza A hemagglutinin (HA) protein.

FIG. 153. Diagram of influenza A nucleoprotein (NP); unconventional nuclear localization signal (NLS), (SEQ ID NO: 183), bipartite NLS, (SEQ ID NO: 184).

FIG. 154. Diagram of influenza A neuraminidase (NA) protein.

FIG. 155. Diagram of influenza A M2 protein.

FIG. 156. Expression of viral HAs; wild type, (SEQ ID NO: 151), H1(1918)ACS (SEQ ID NO: 152), H5ΔPS (SEQ ID NO: 153), and H5ΔPS2 (SEQ ID NO: 154).

FIG. 157. Humoral and cellular immune responses to 1918 influenza HA after DNA vaccination.

FIG. 158. Immune protection conferred against lethal challenge of 1918 influenza and lack of T cell dependence.

FIG. 159. Immune mechanism of protection showing dependence on Ig.

FIG. 160. Development of HA-pseudotyped lentiviral vectors.

FIG. 161. VRC 7720: CMV/R(8κb)Influenza H5(A/Thailand/1(KAN-1)/2004) HA/h, (SEQ ID NO: 161).

FIG. 162. VRC 7721: CMV/R(8κB)Influenza H5(A/Thailand/1(KAN-1)/2004) HA mutA/h, (SEQ ID NO: 162).

FIG. 163. VRC 7722: CMV/R 8κB Influenza A/New Caledonia/20/99(H1N1) wt, (SEQ ID NO: 163).

FIG. 164. VRC 7723 (VRC 7727): CMV/R 8κB Influenza A/New Caledonia/20/99(H1N1) mut a, (SEQ ID NO: 164).

FIG. 165. VRC 7724: CMV/R 8κB Influenza A/Wyoming/3/03 (H3N2)wt, (SEQ ID NO: 165).

FIG. 166. VRC 7725 (VRC 7729): CMV/R 8κB Influenza A/Wyoming/3/03 (H3N2) mut a, (SEQ ID NO: 166).

FIG. 167. Sequence alignment of CMV/R and CMV/R 8κB Promoters.

FIG. 168. Amino acid sequence alignment of VRC 7721 and VRC 7720 inserts.

FIG. 169. Intracellular flow cytometric analysis of gp145 env-specific CD4+ and CD8+ T-cell responses of immunized mice.

FIG. 170. End-point dilutions of antibody responses in mice vaccinated with wild-type CMV/R or CMV/R 8κb plasmid DNA expressing HIV gp145.

FIG. 171. Protective immunity to lethal H5N1 Influenza challenge in mice vaccinated with a CMV/R 8κB plasmid DNA vector expressing H5 Hemagglutinin.

FIG. 172. Schematic diagram of pseudotyped lentiviral reporter assay.

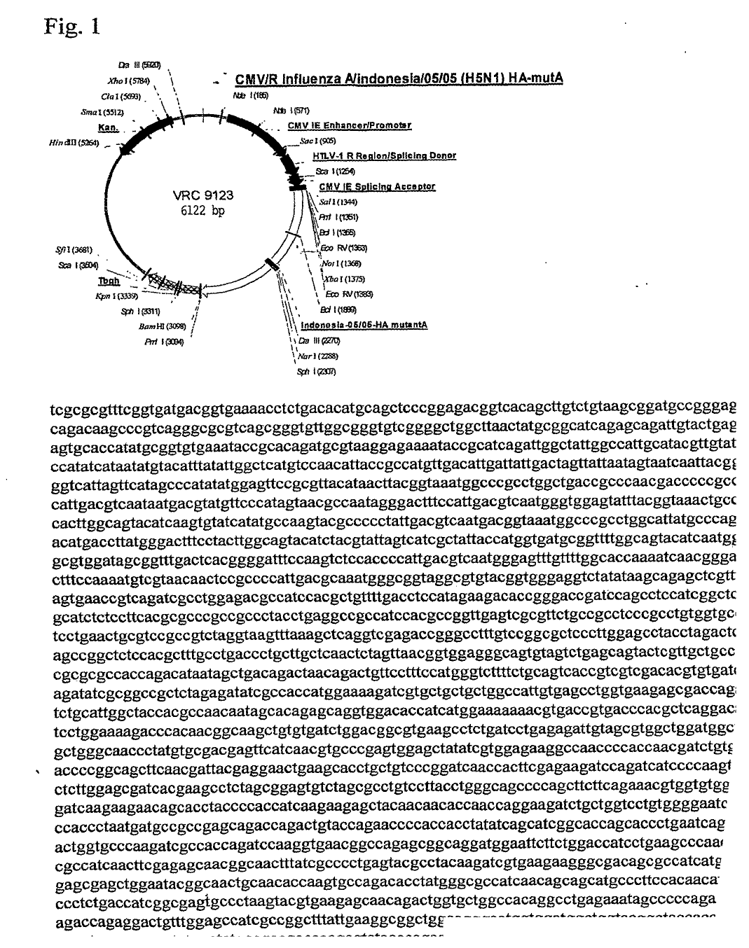

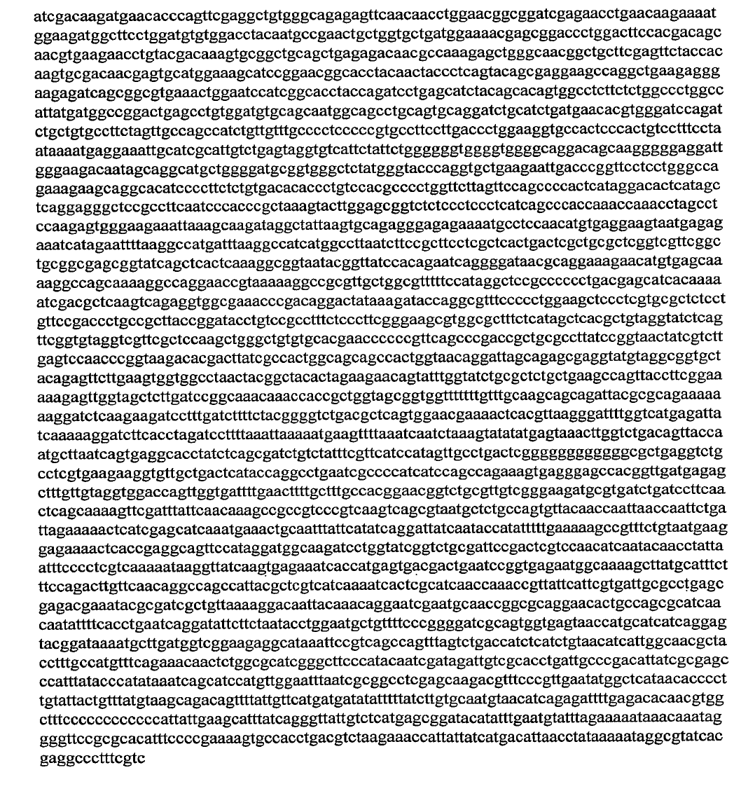

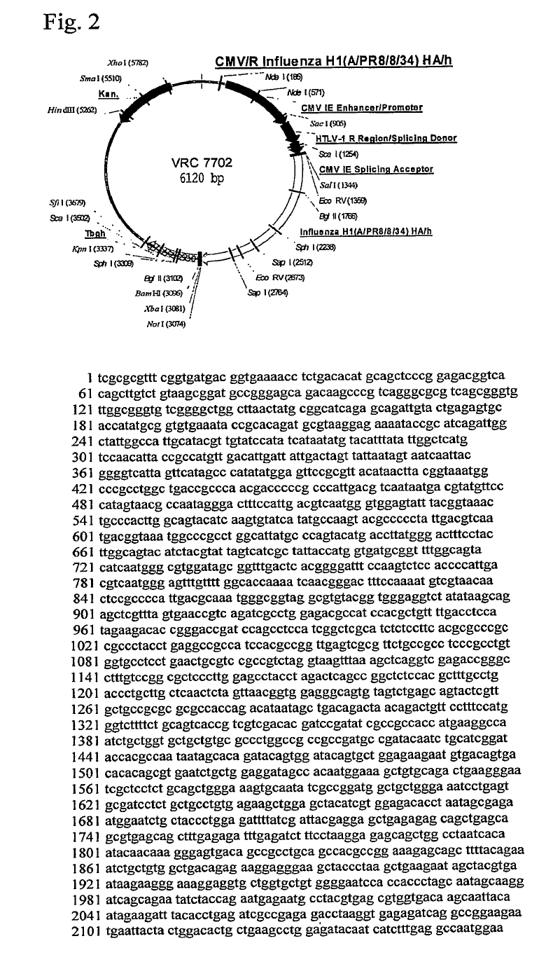

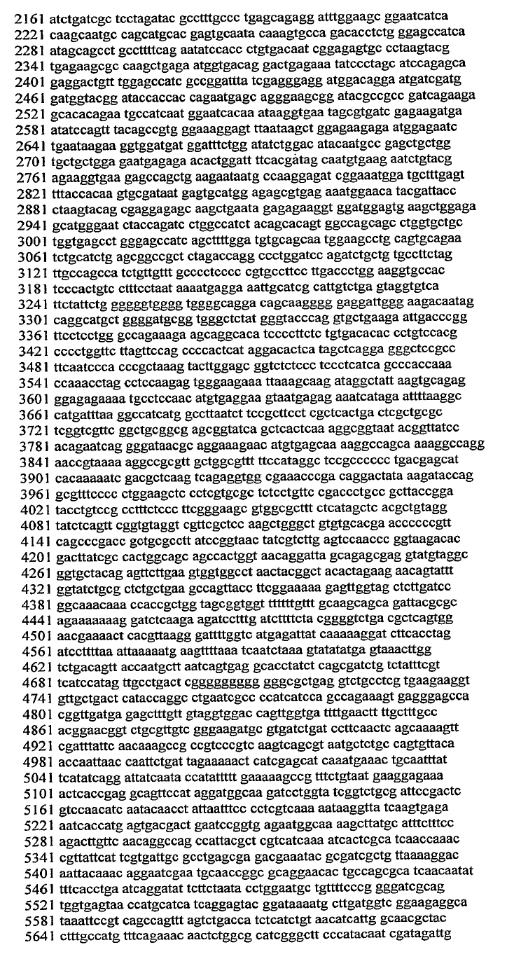

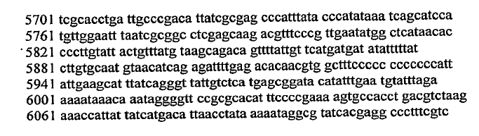

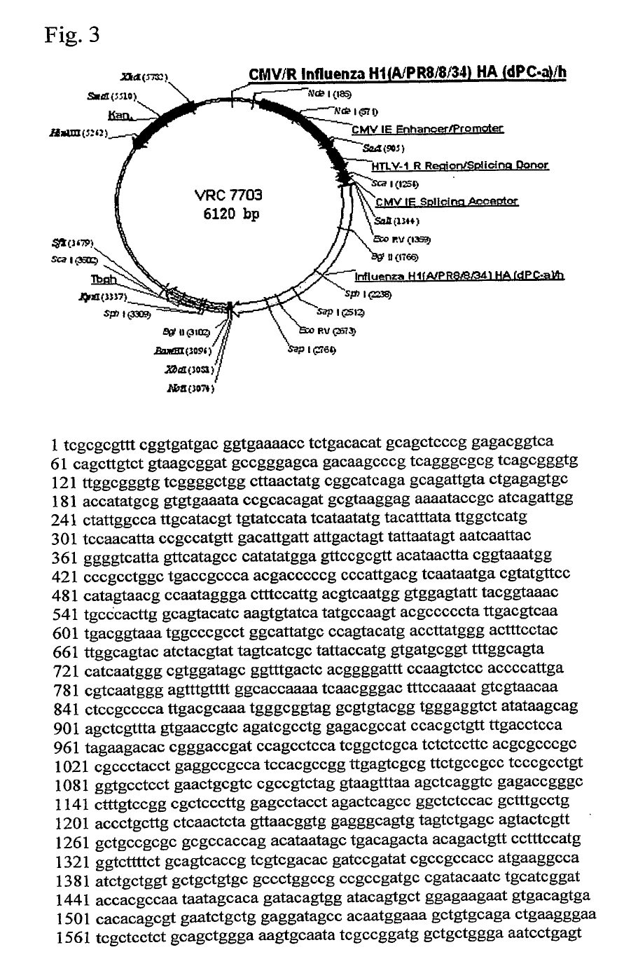

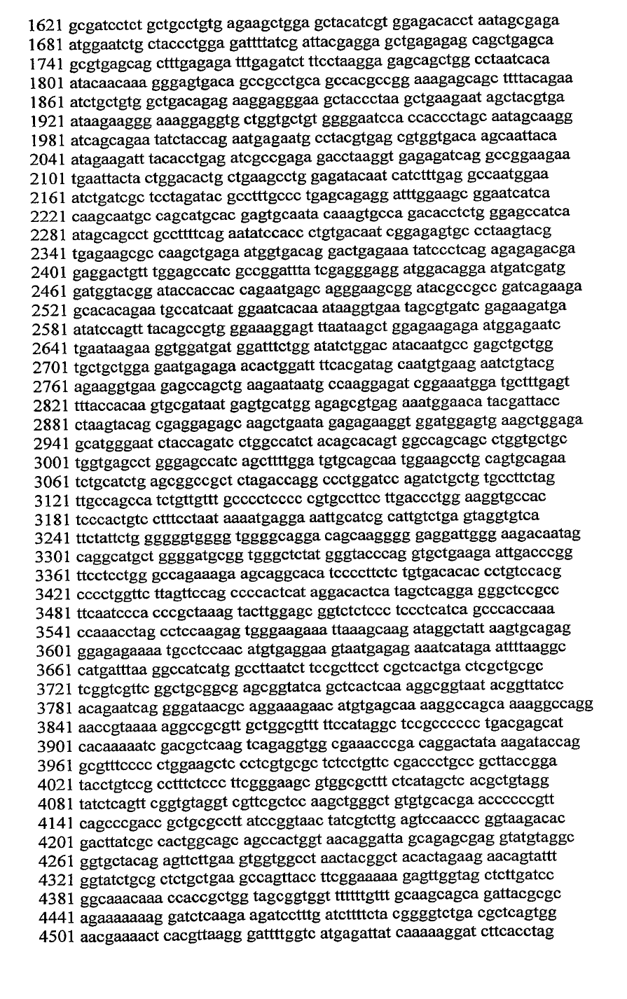

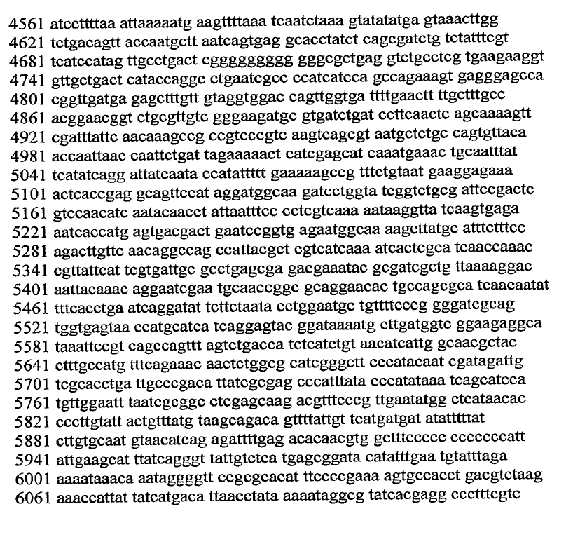

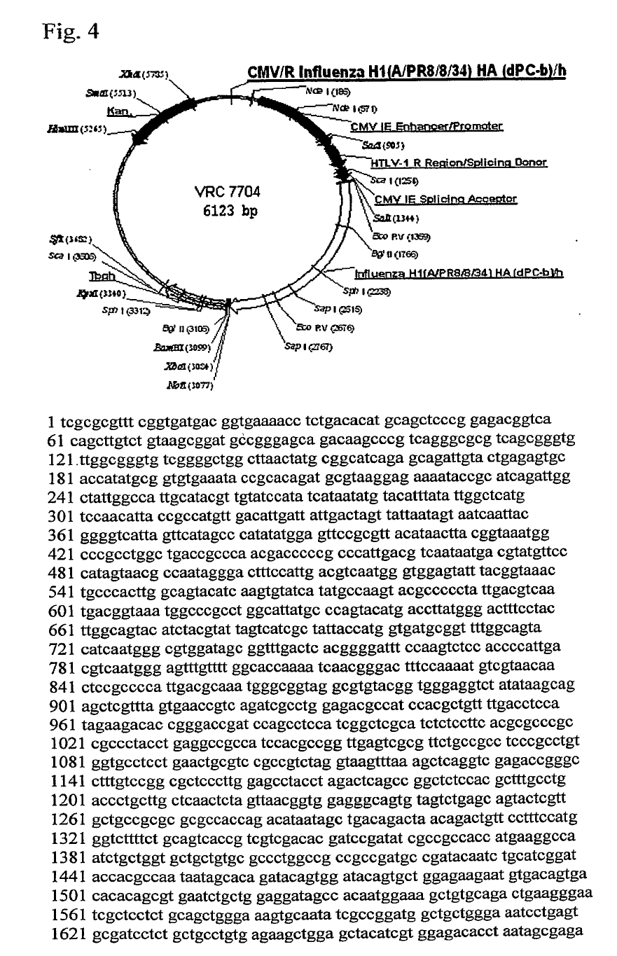

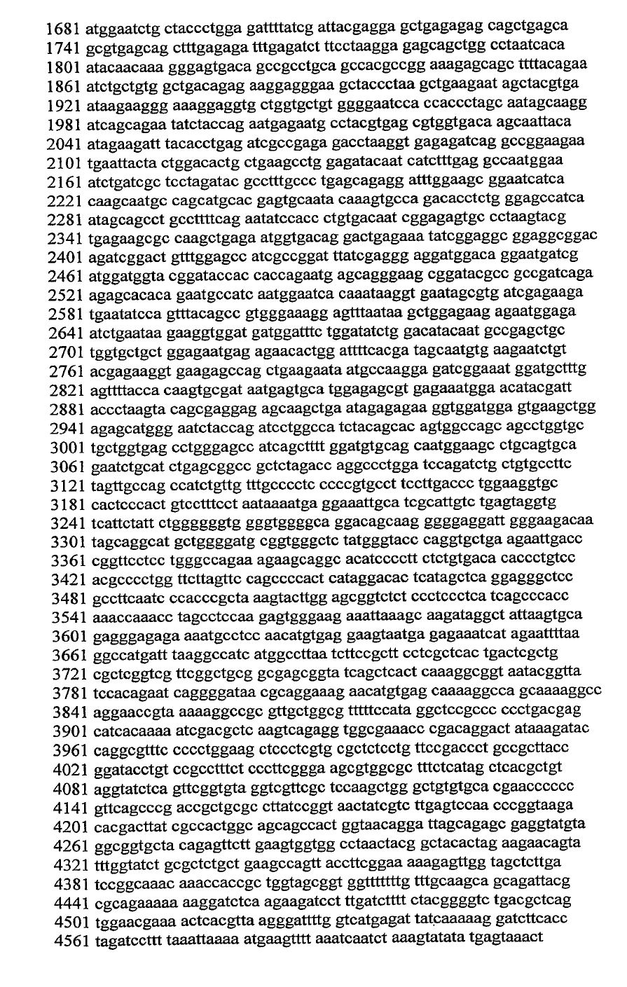

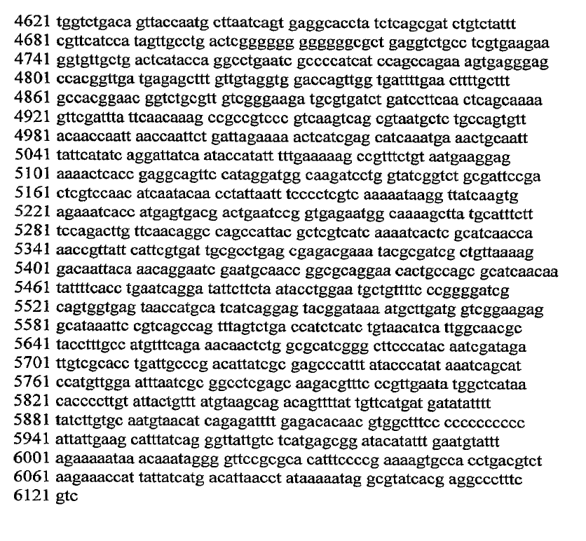

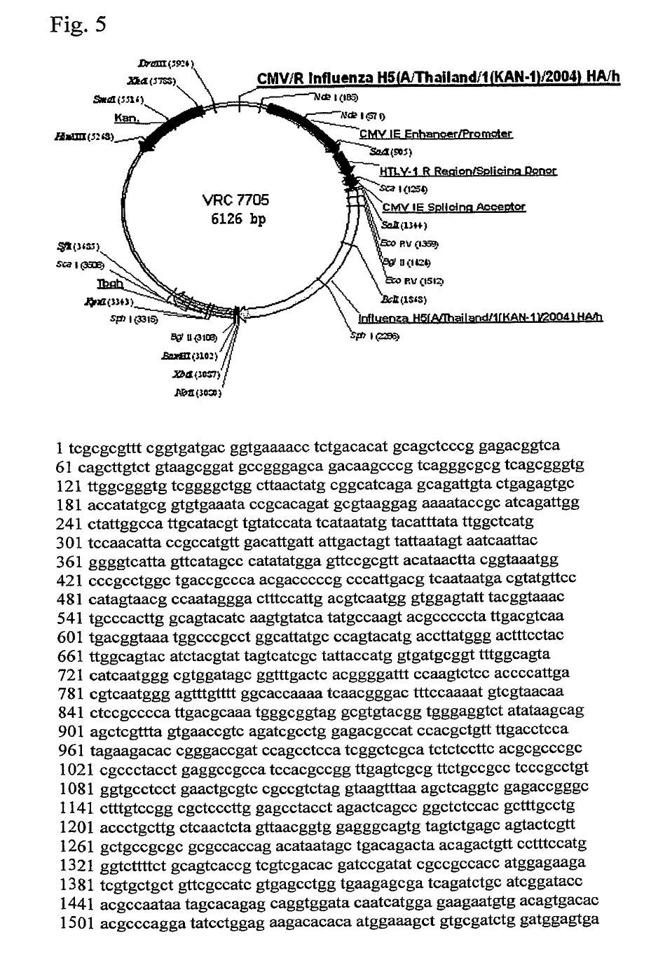

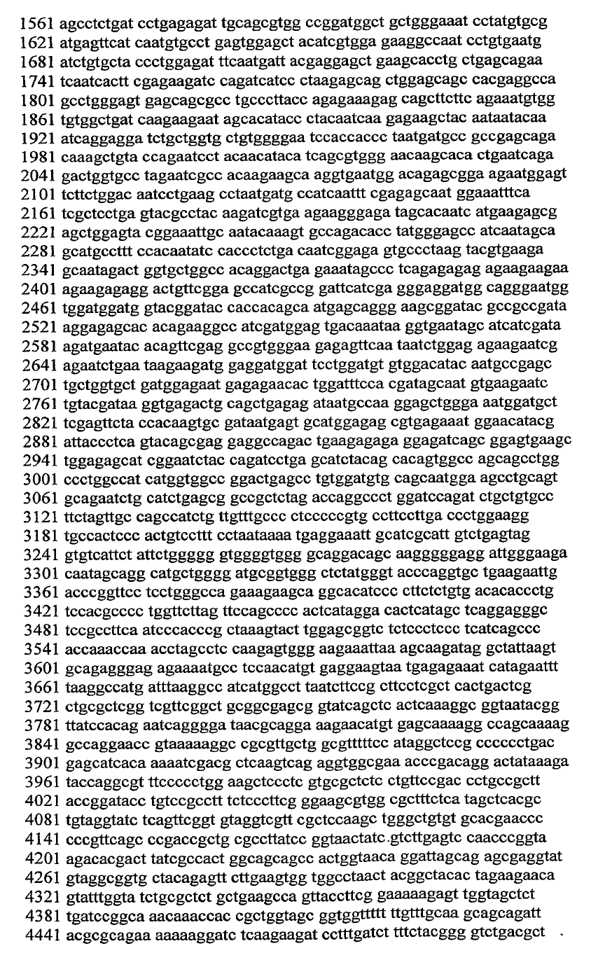

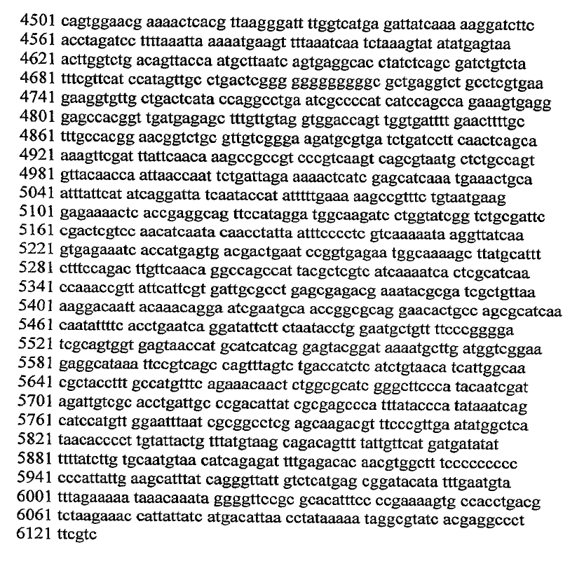

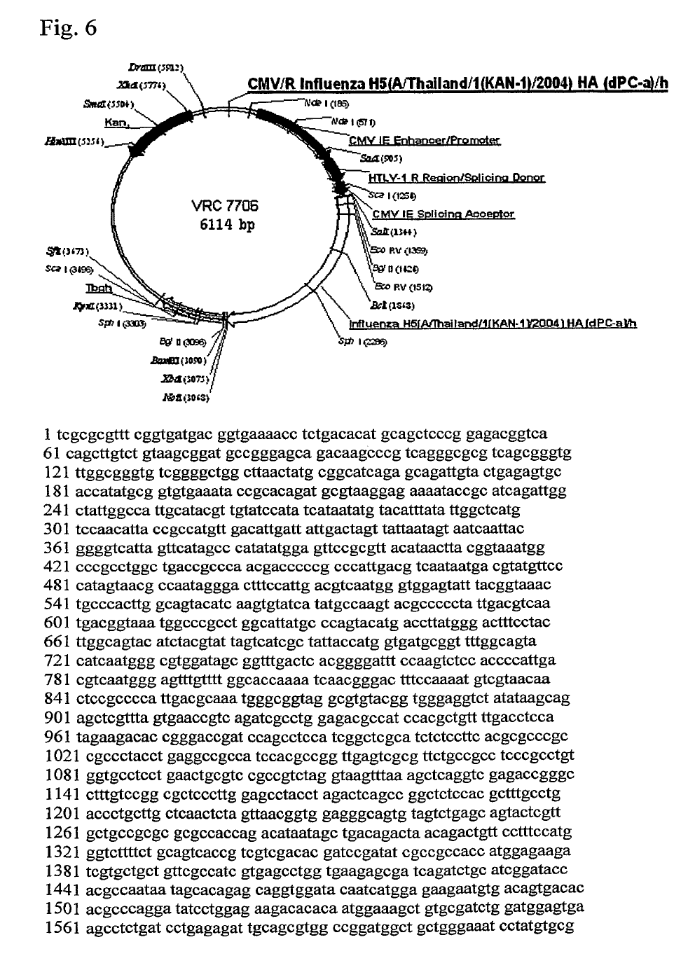

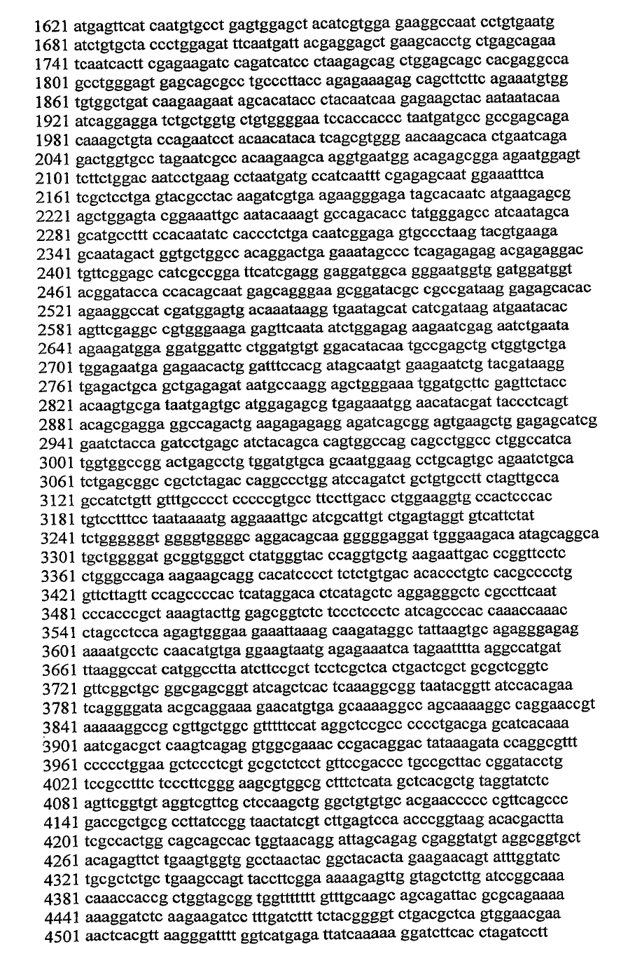

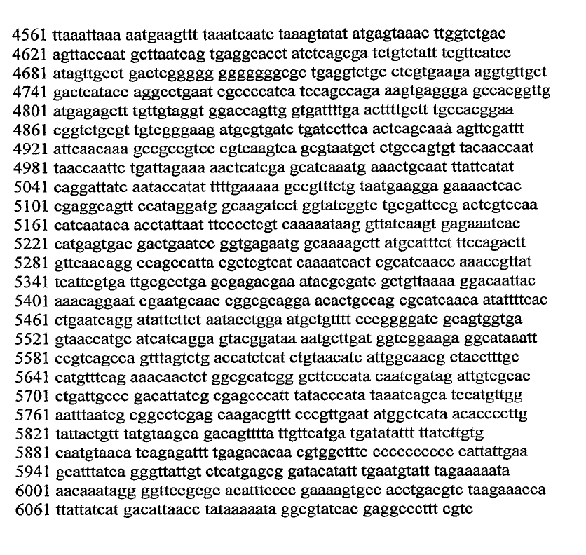

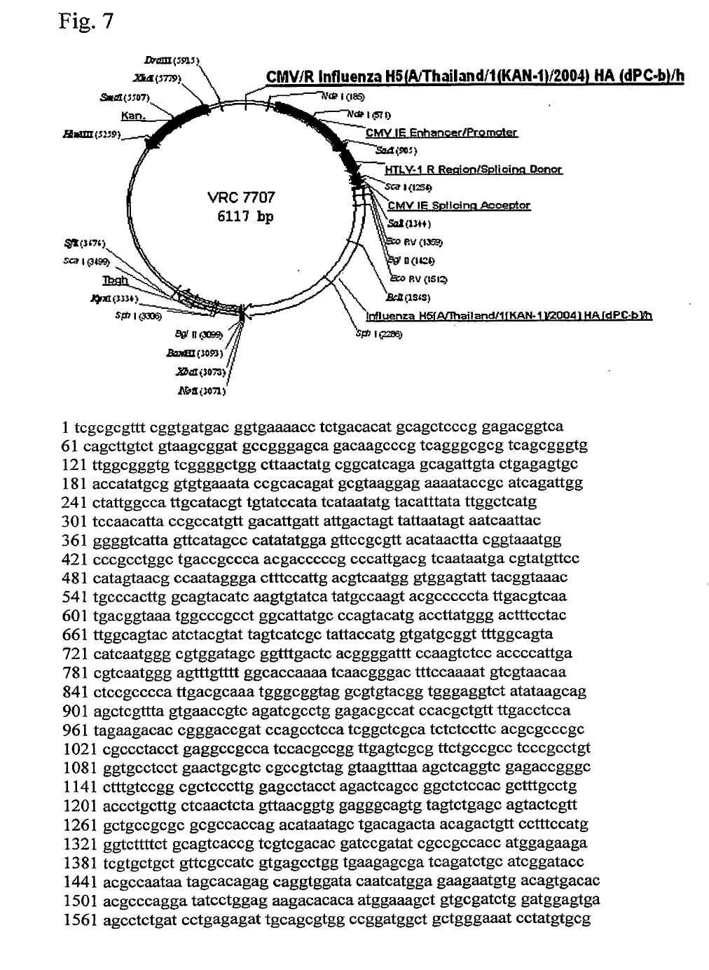

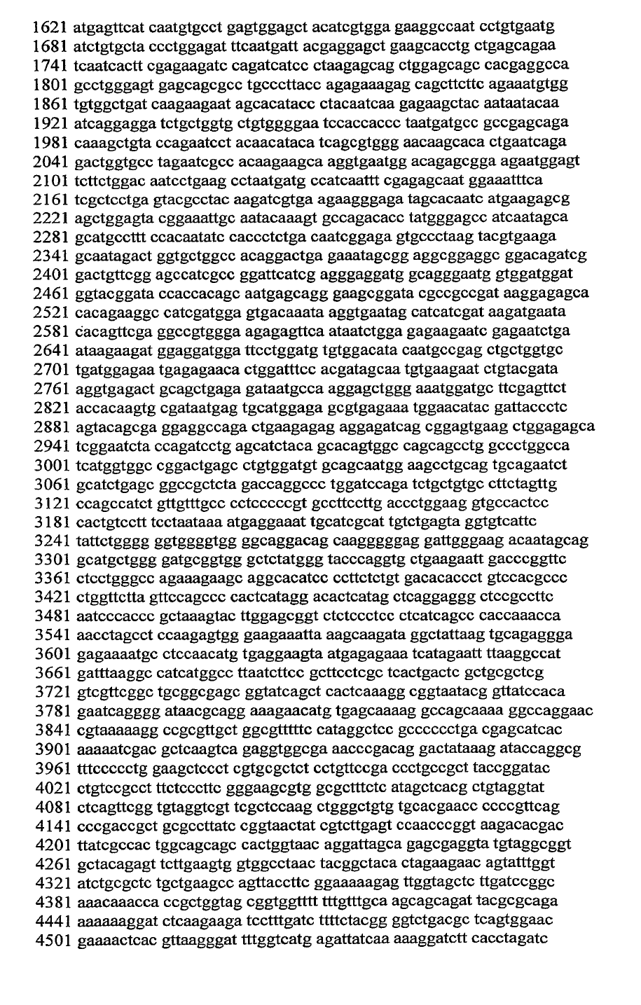

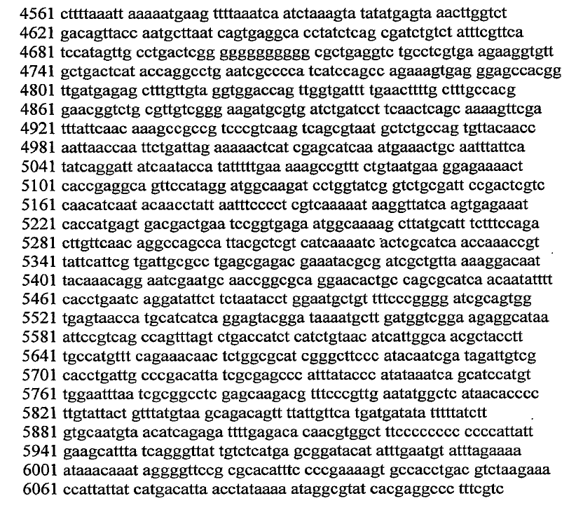

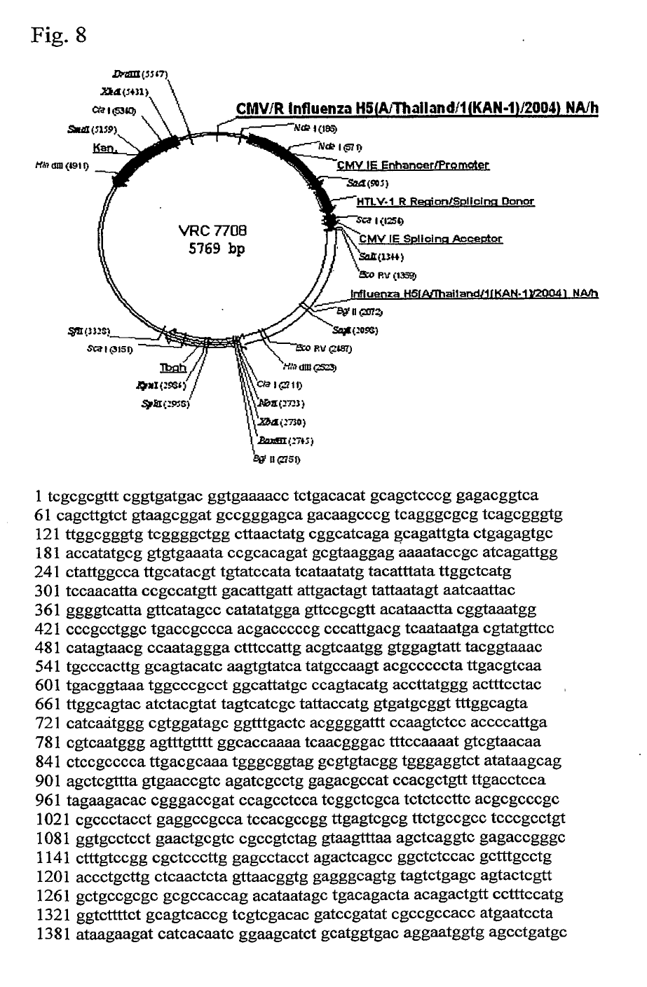

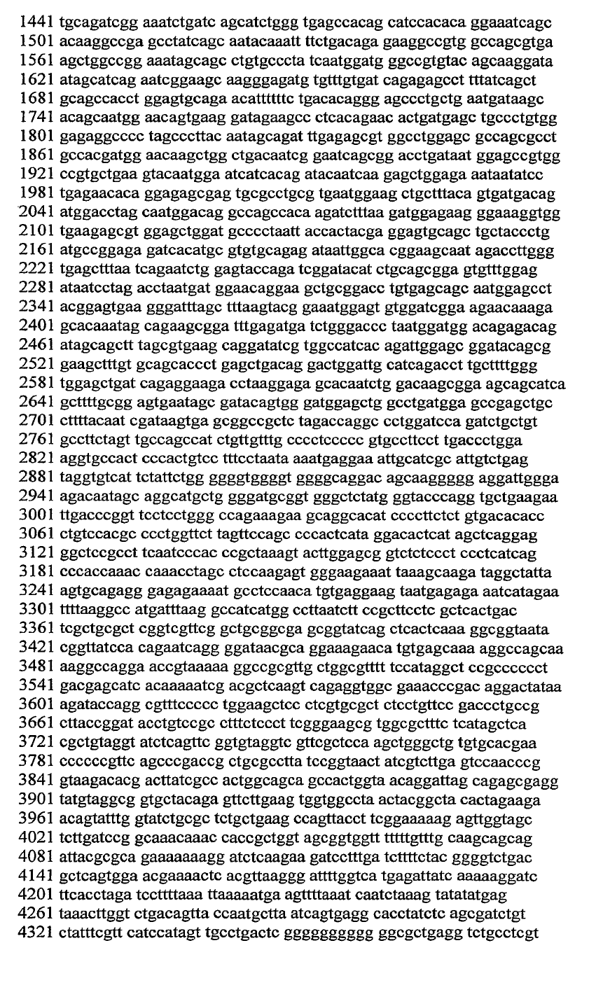

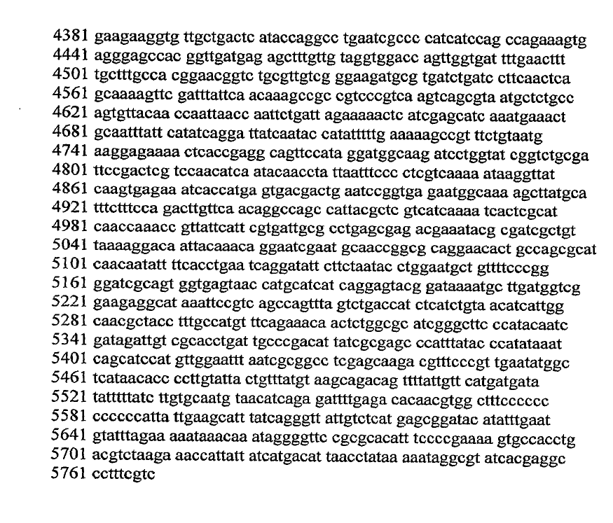

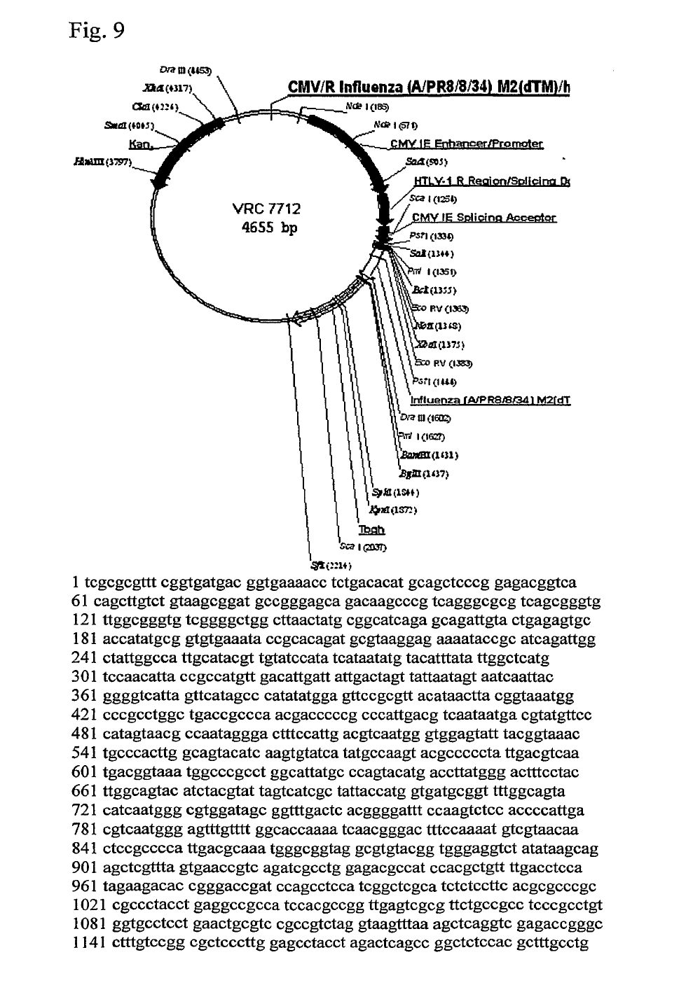

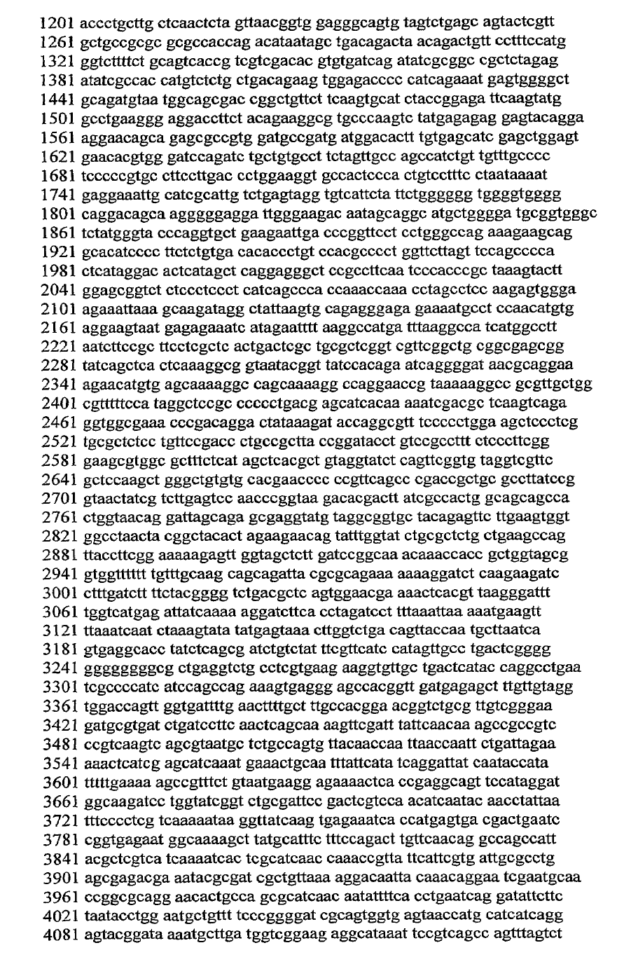

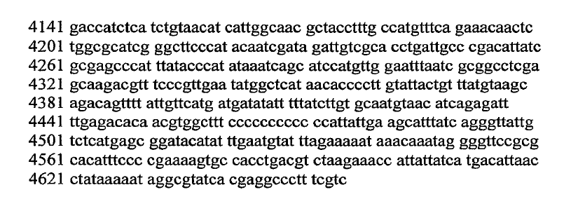

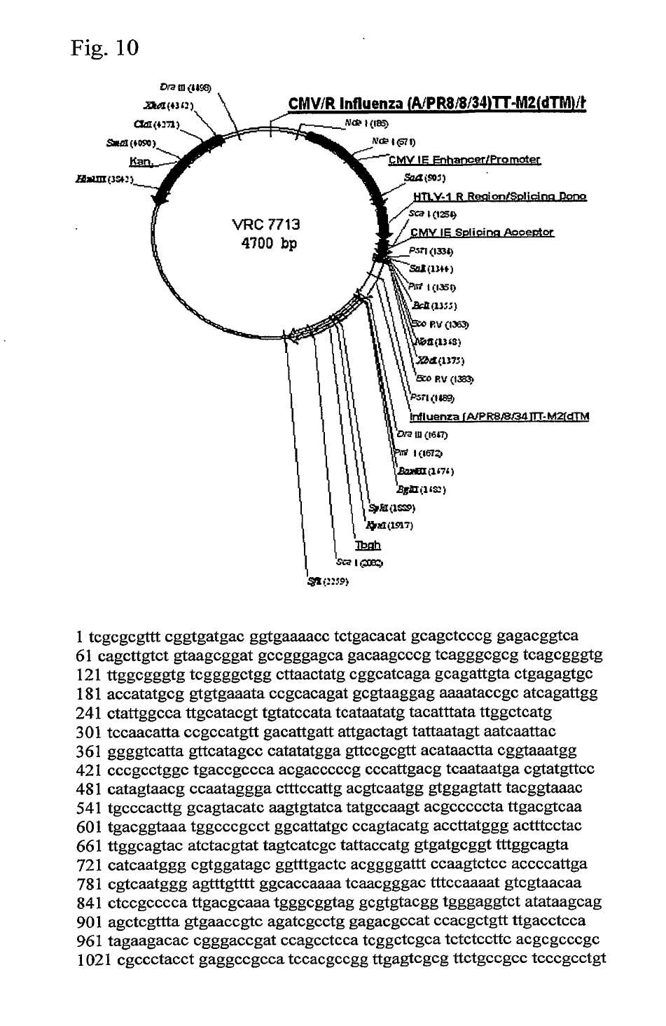

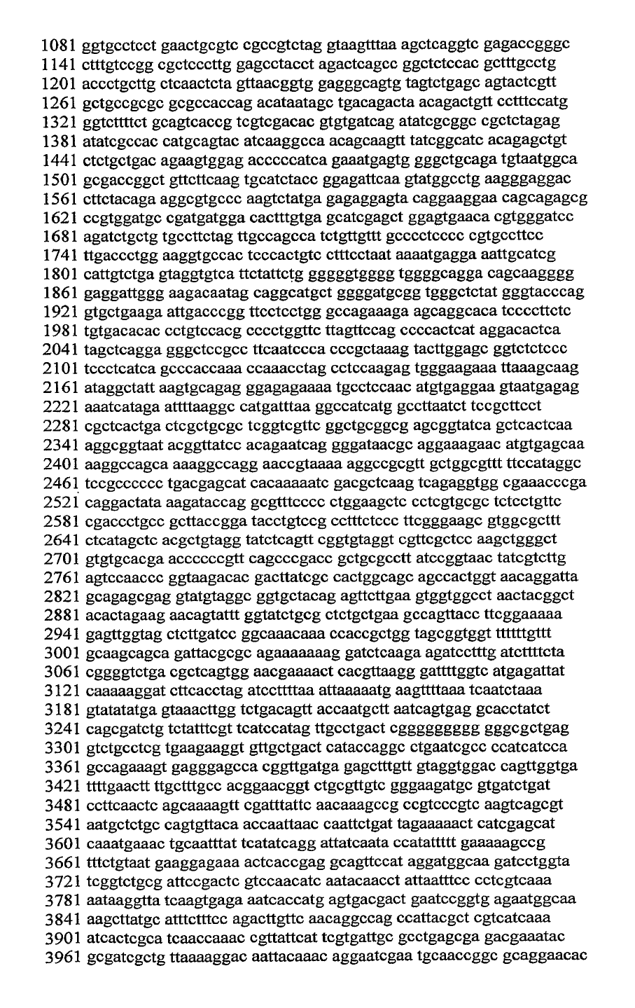

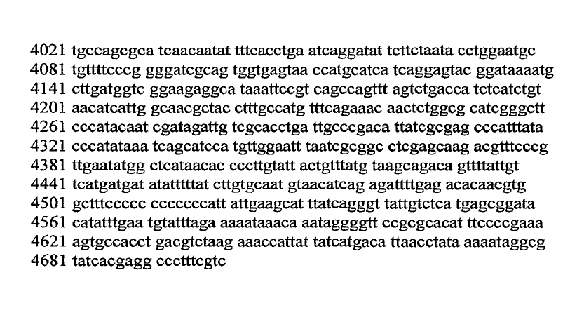

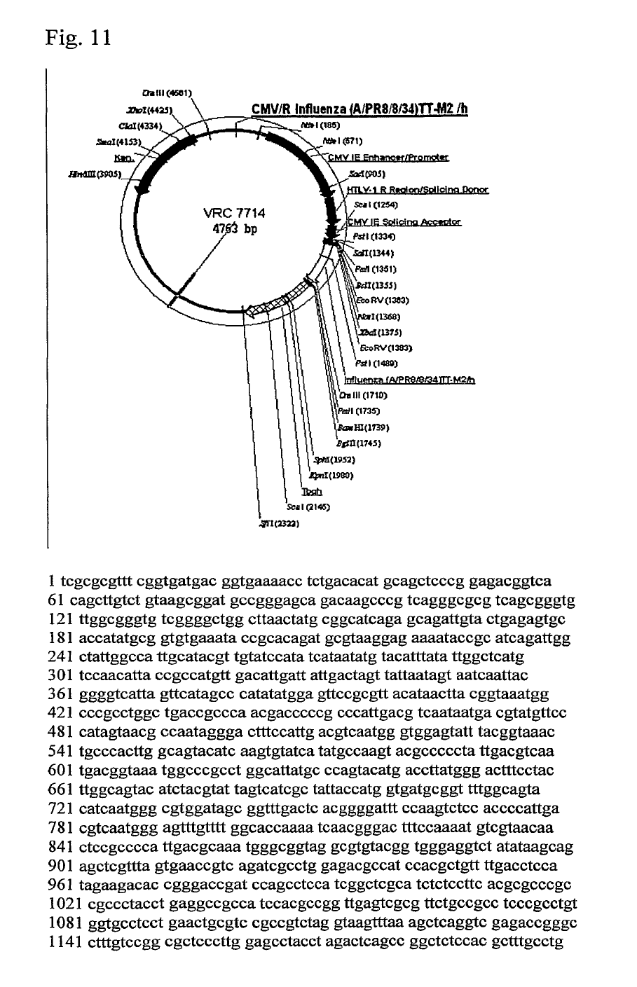

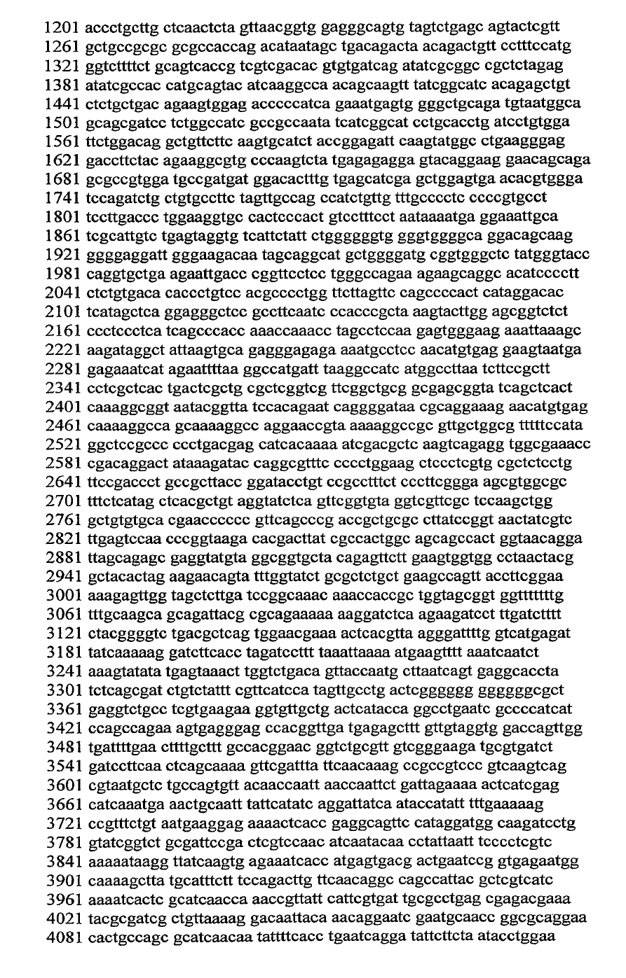

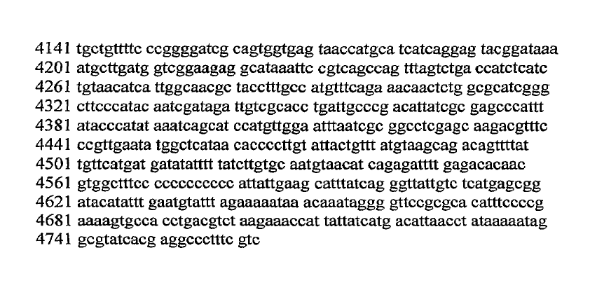

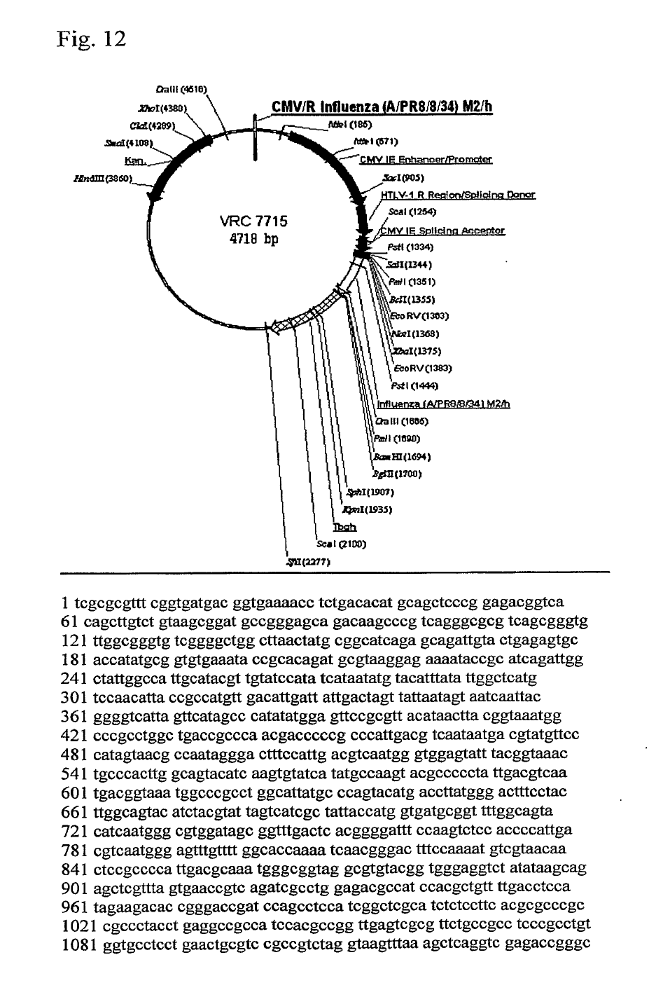

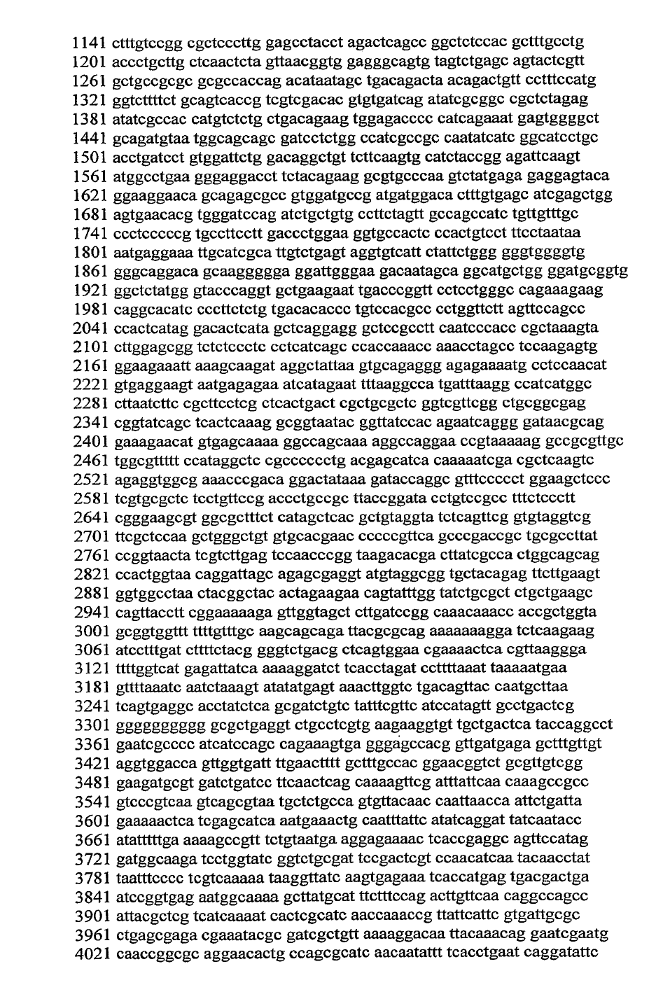

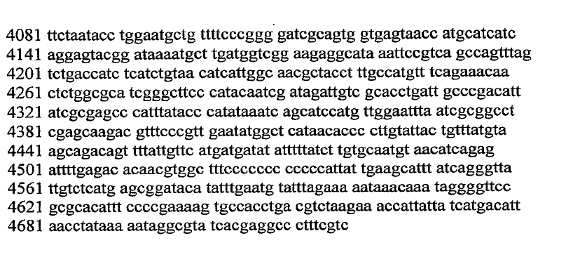

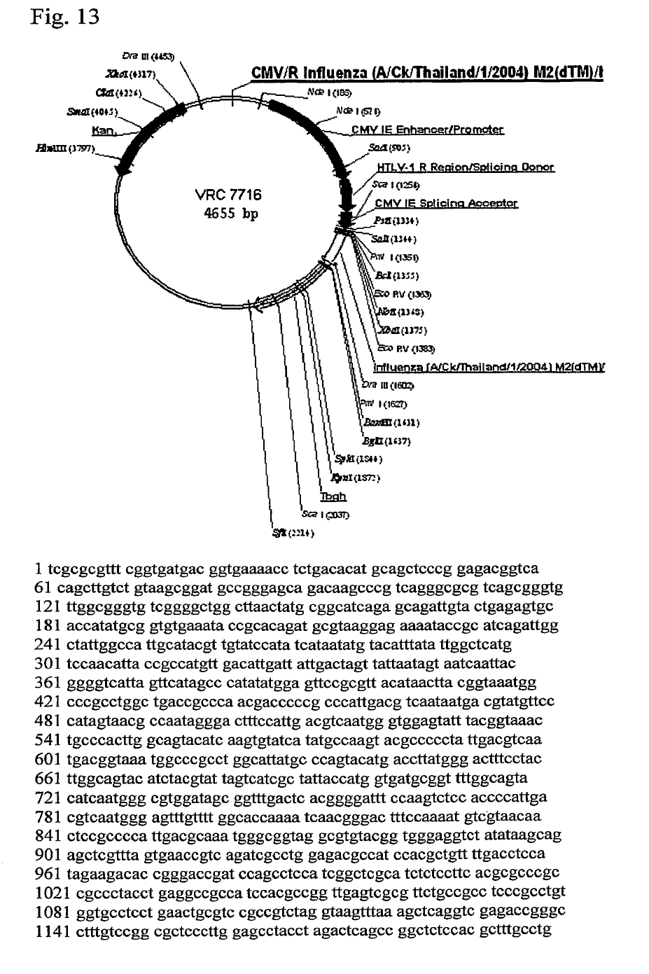

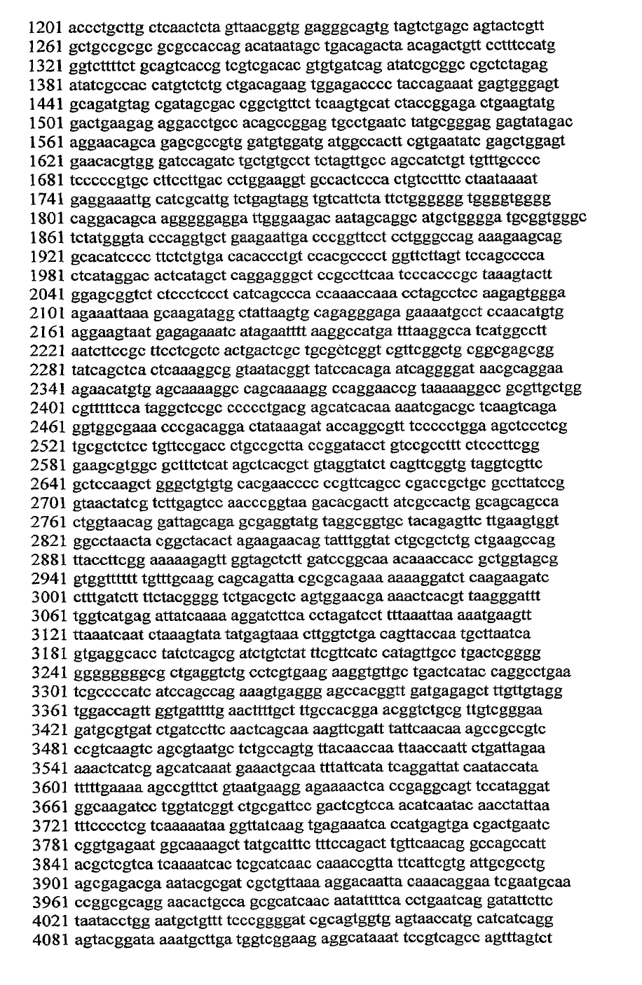

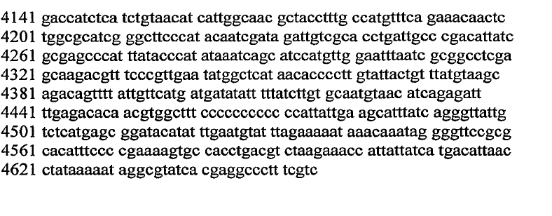

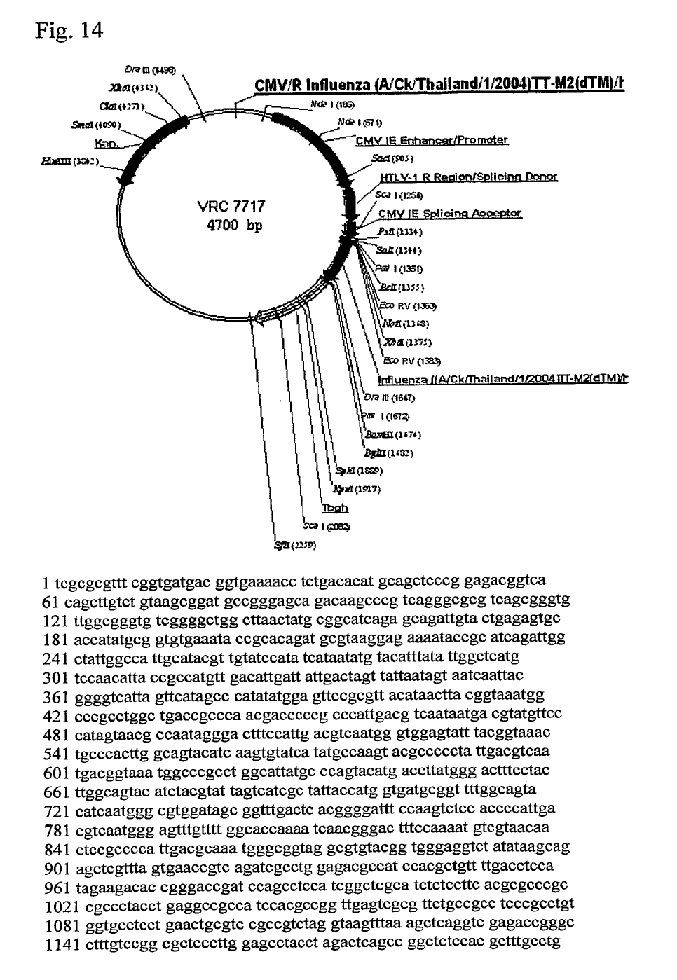

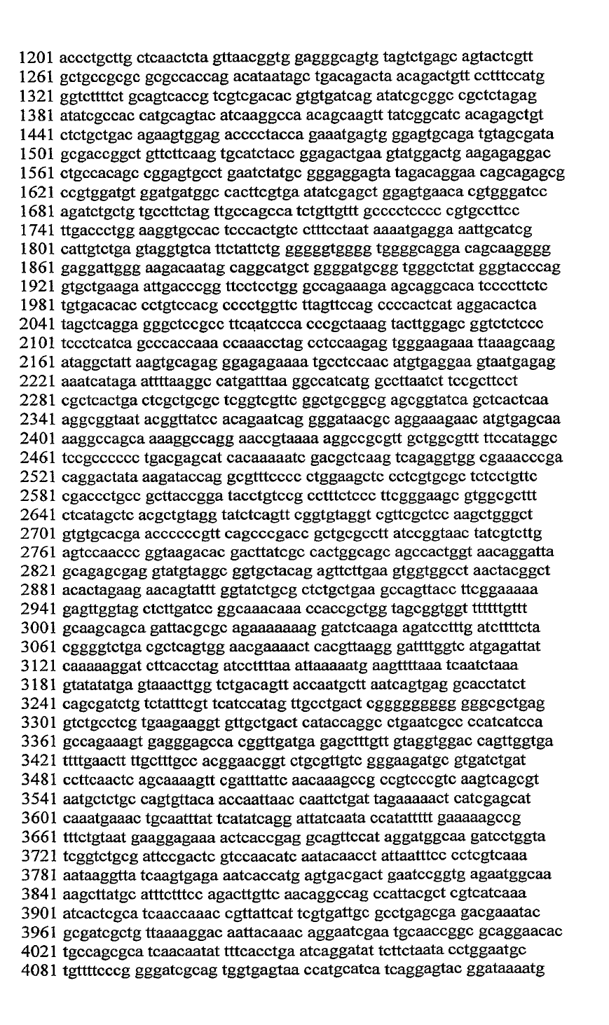

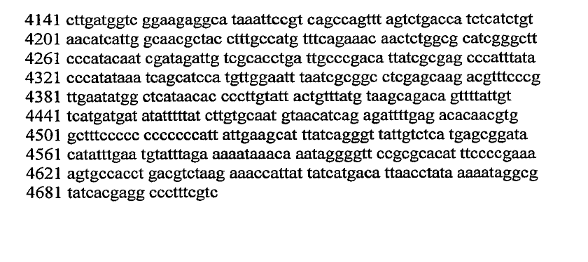

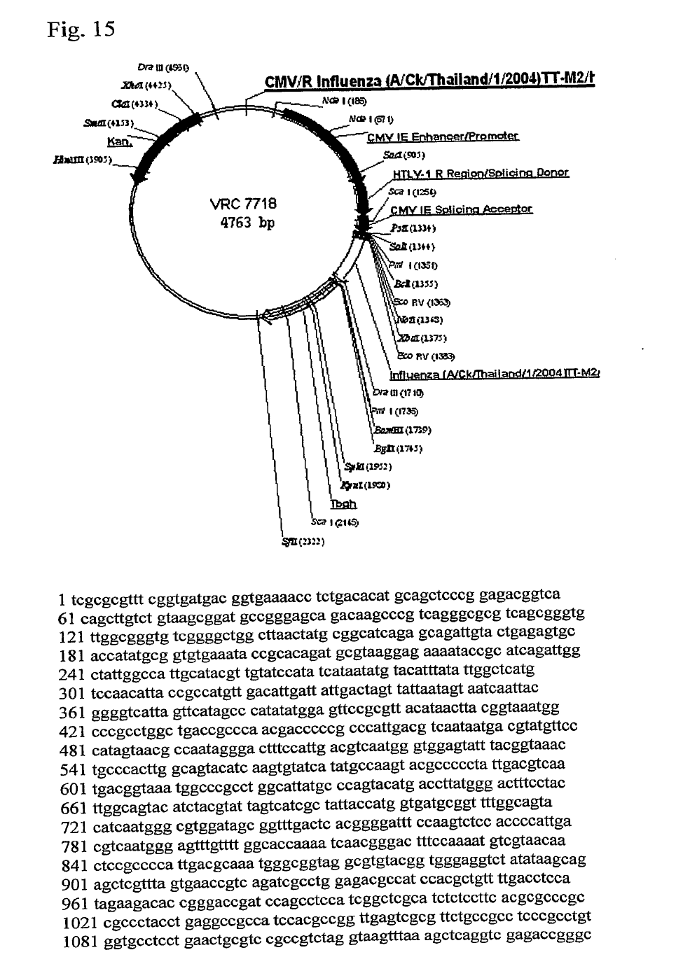

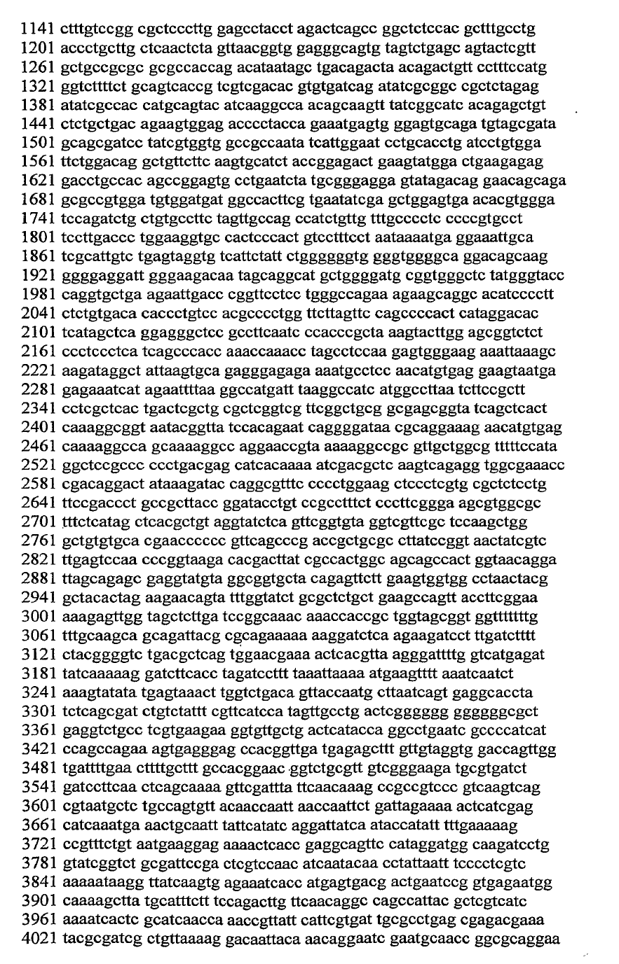

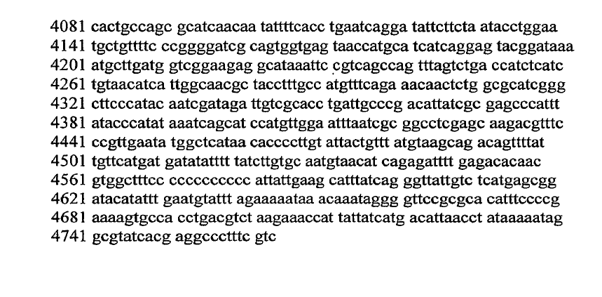

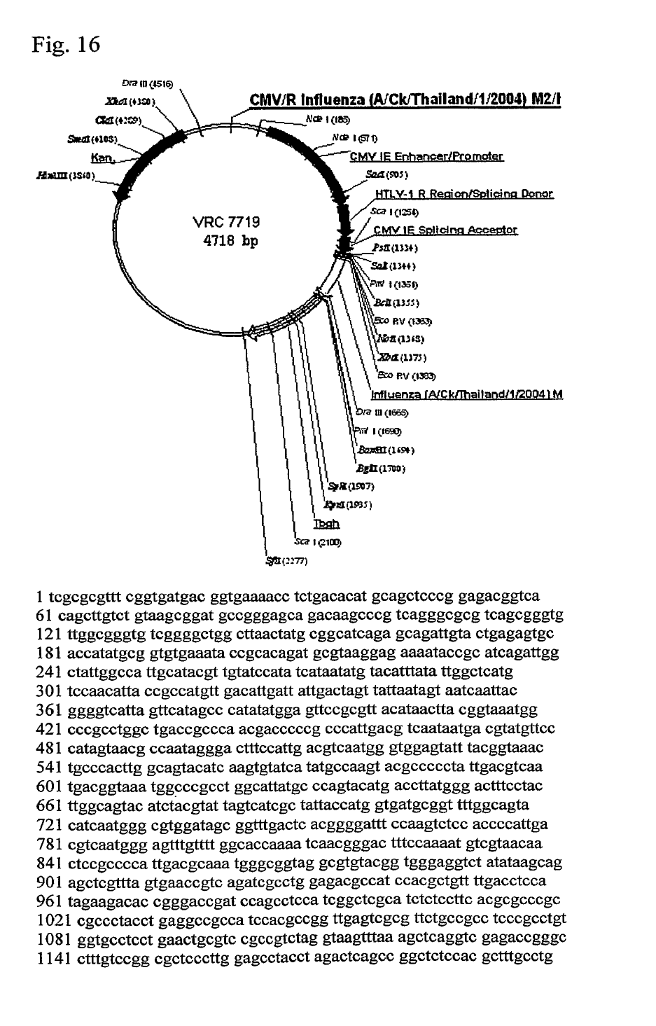

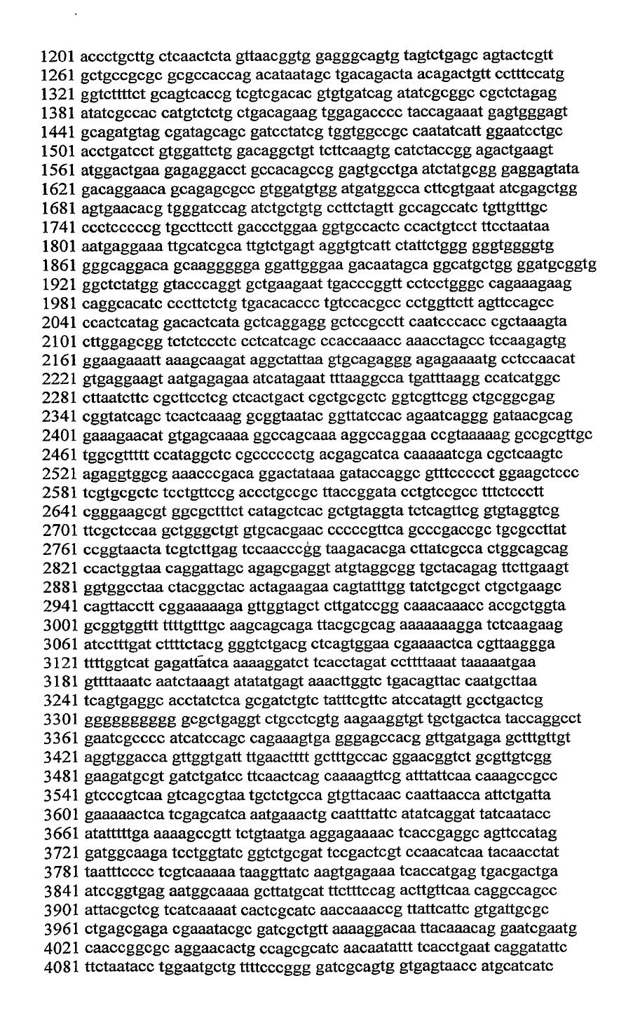

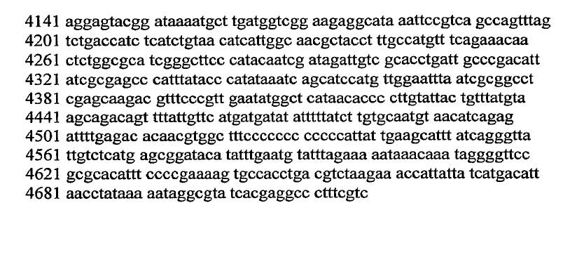

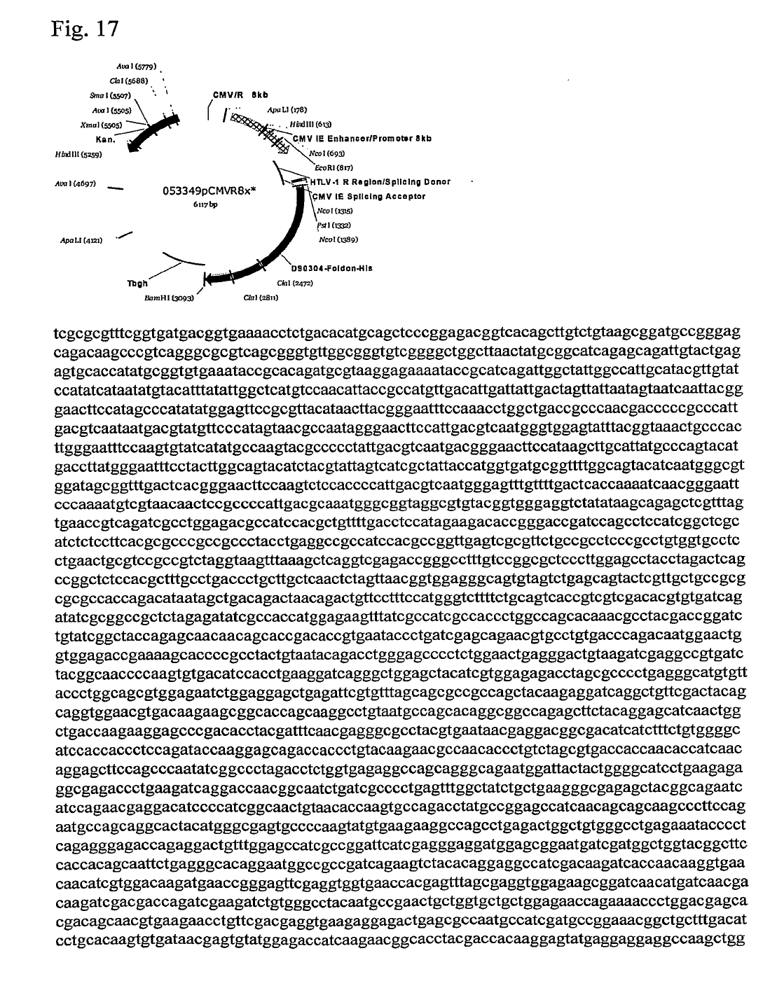

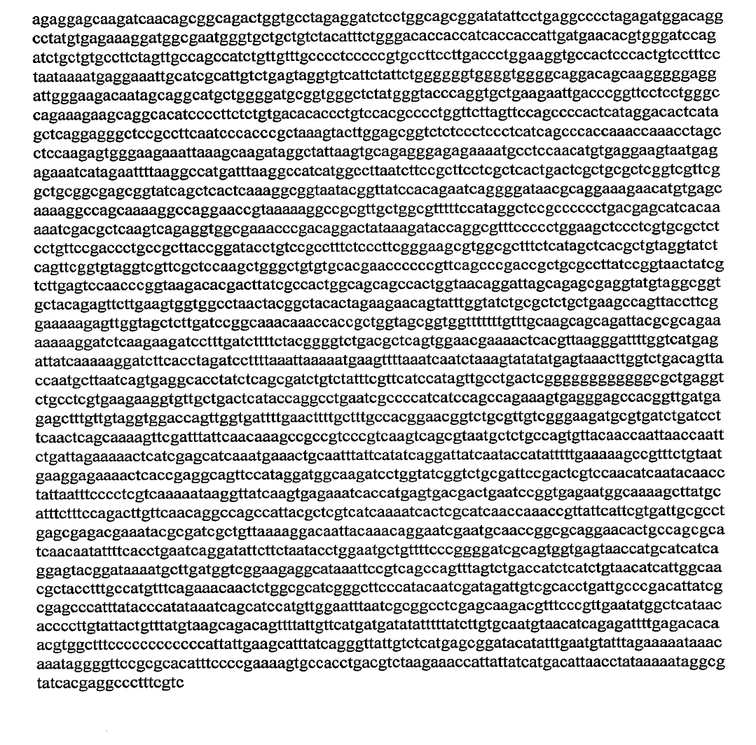

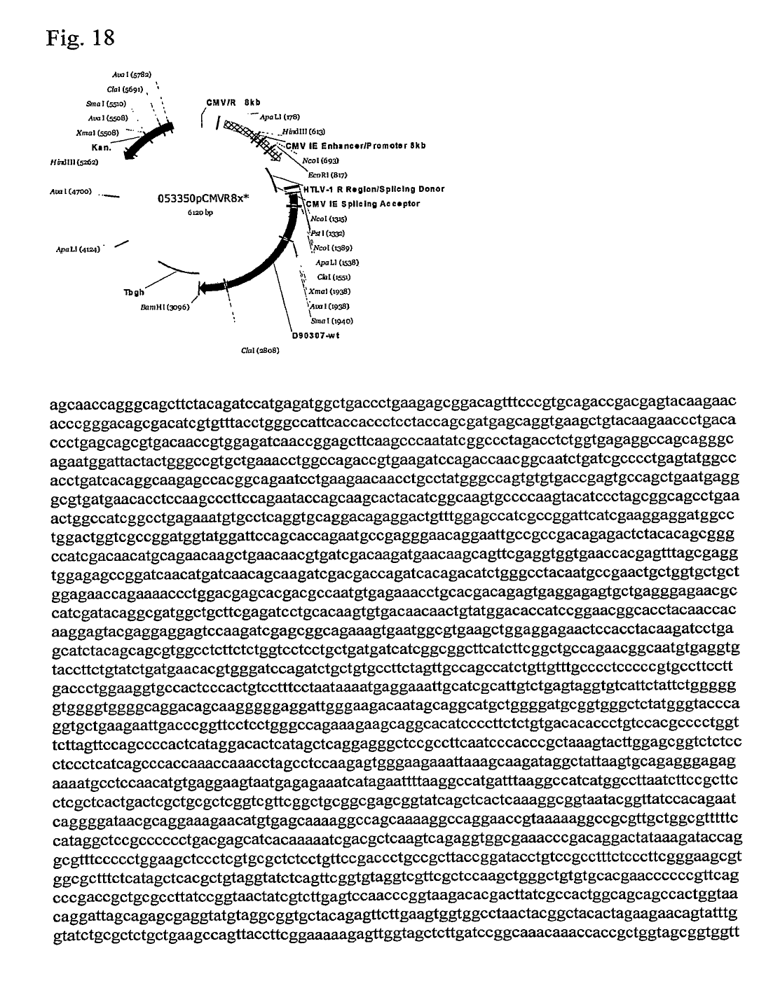

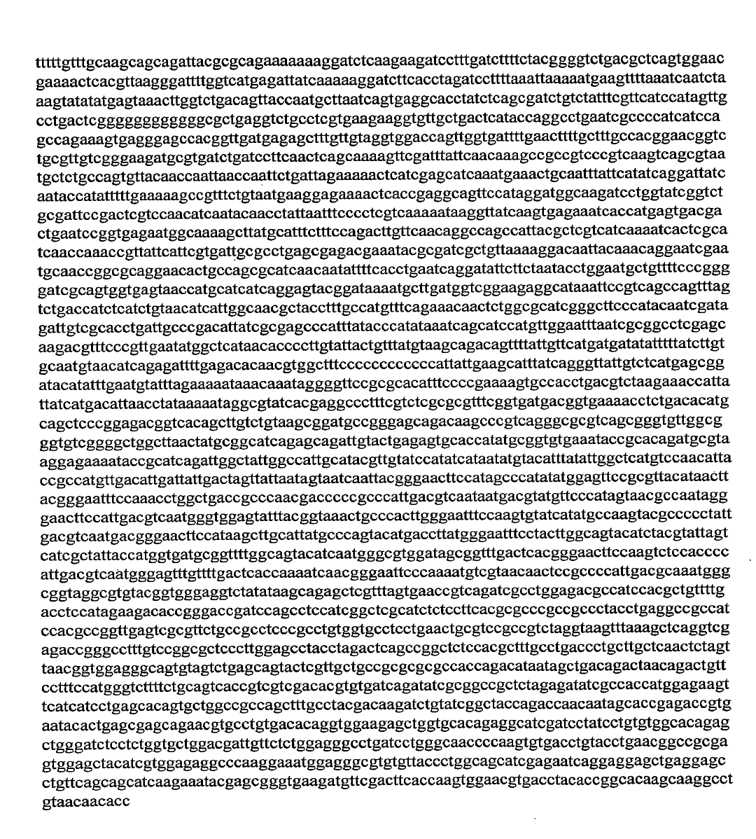

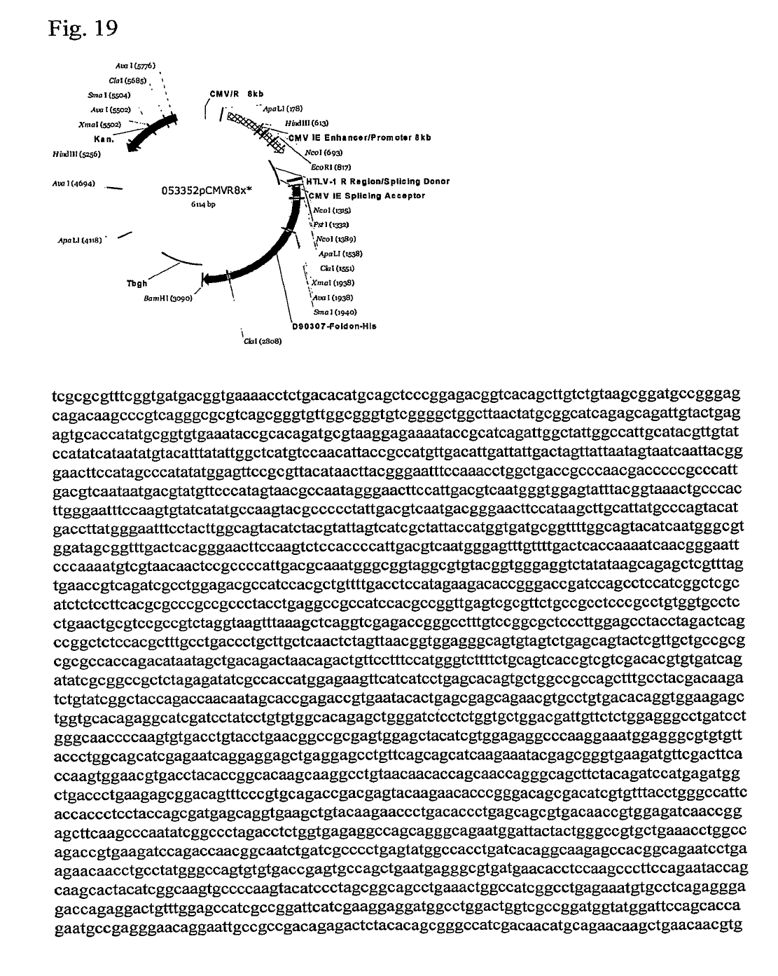

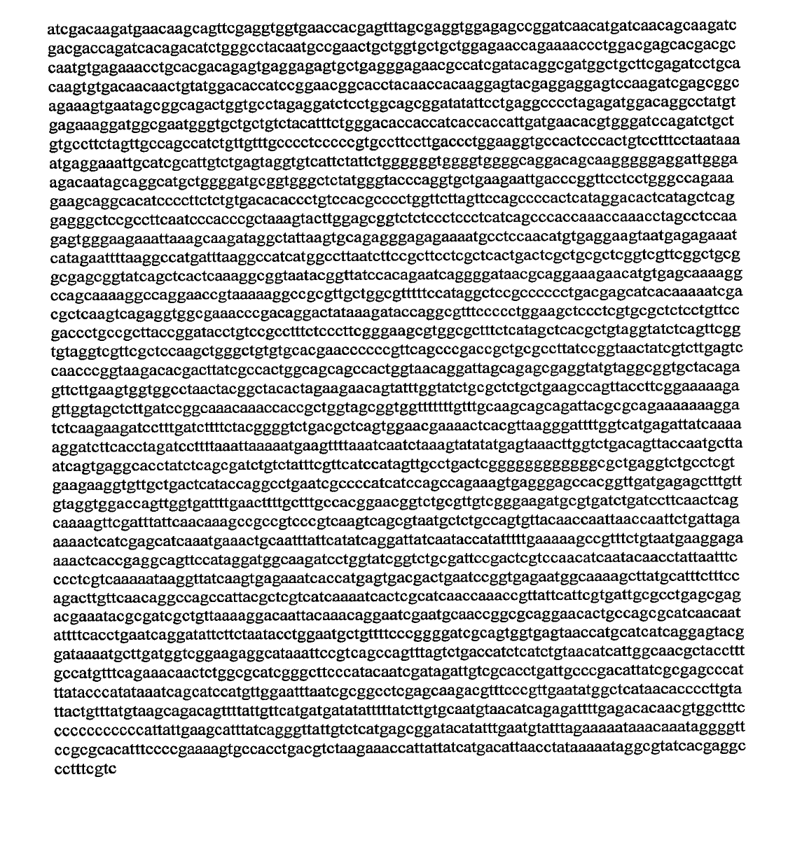

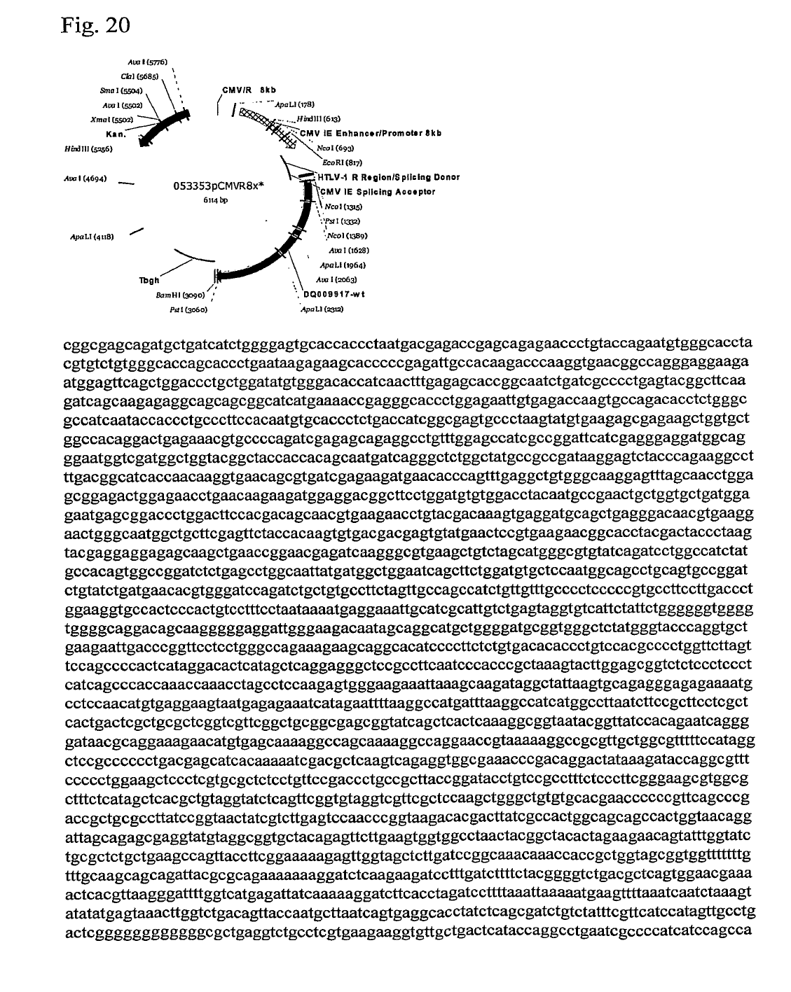

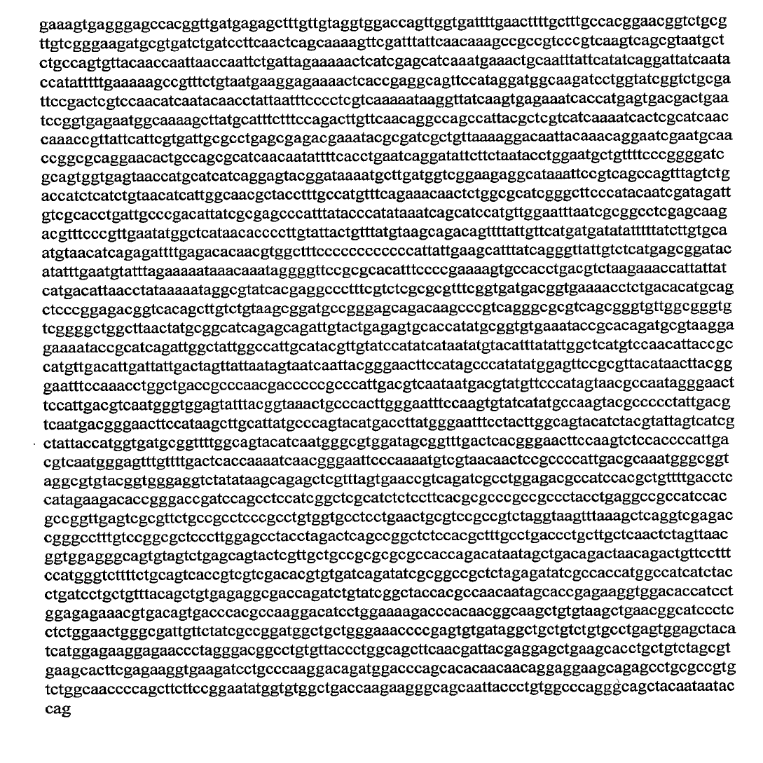

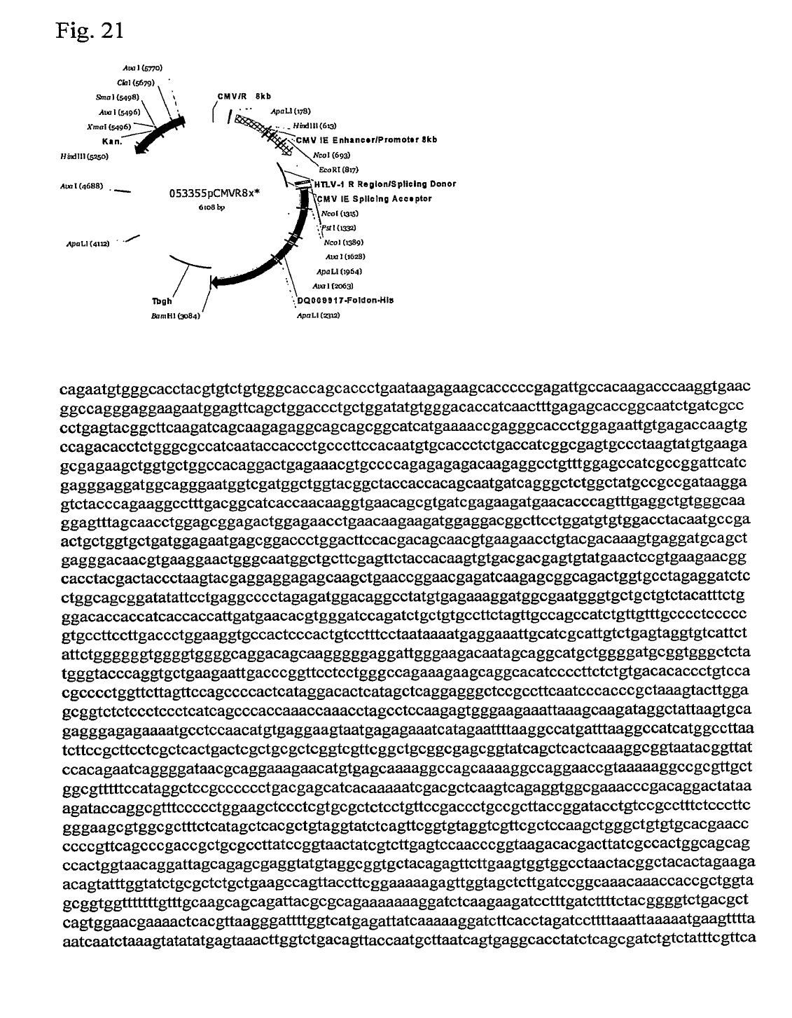

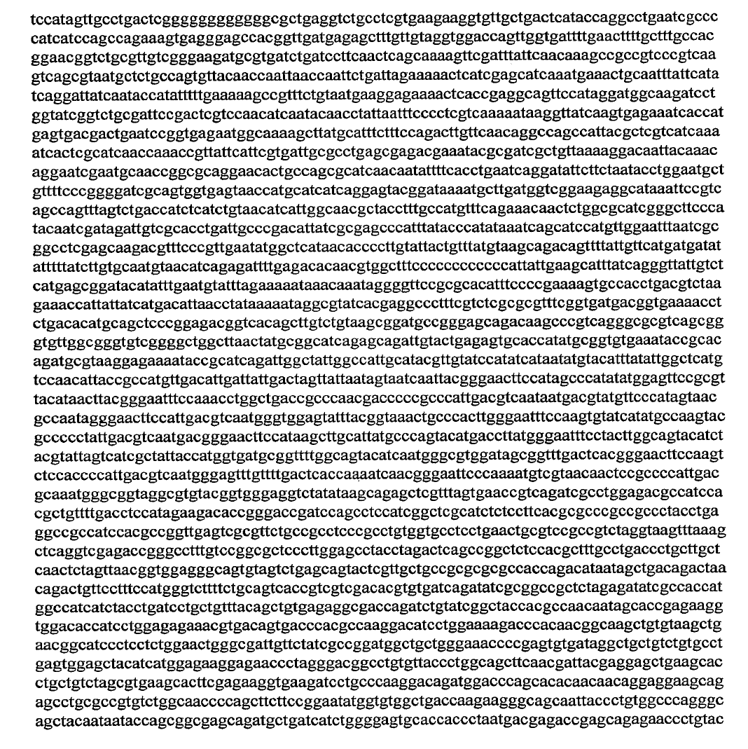

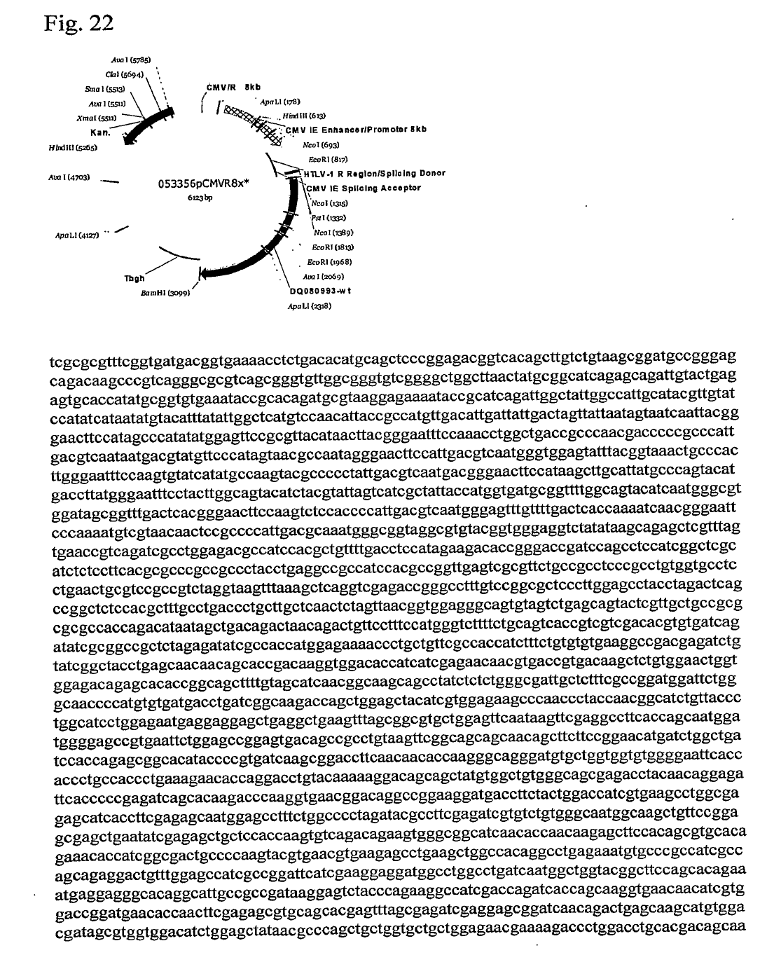

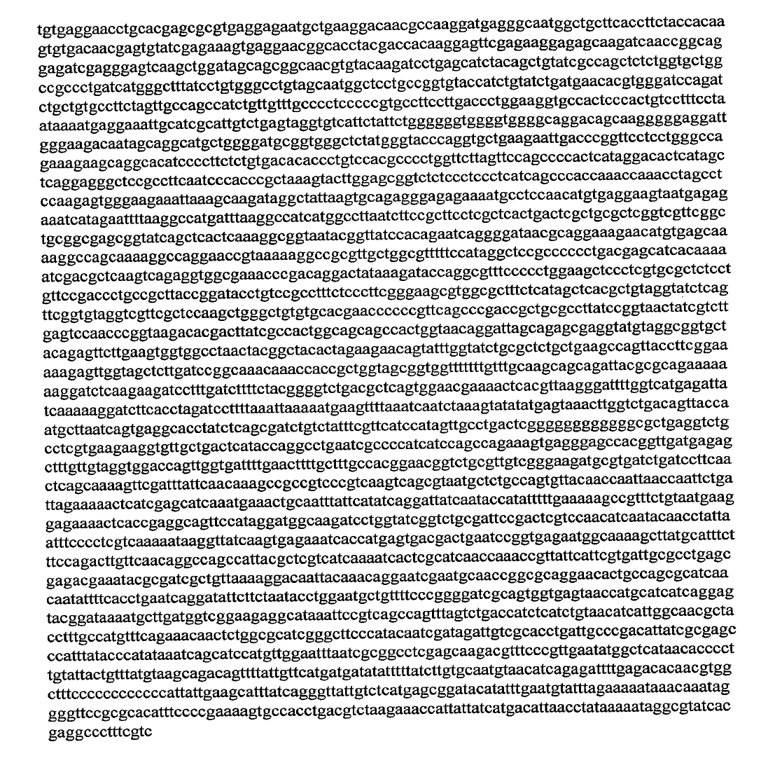

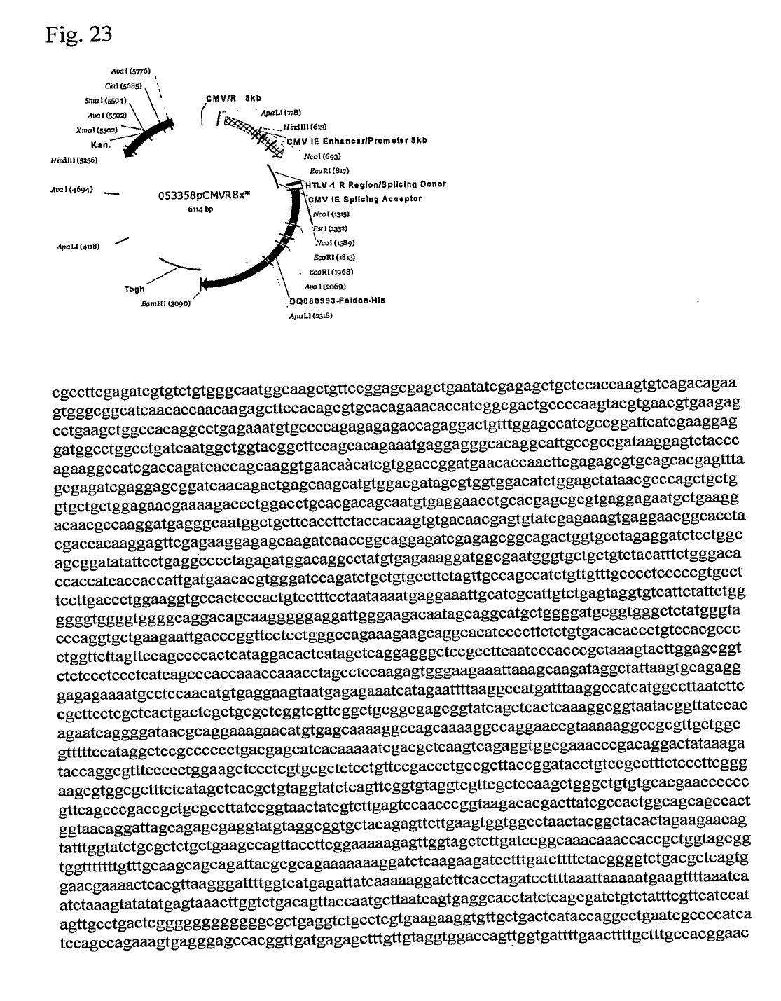

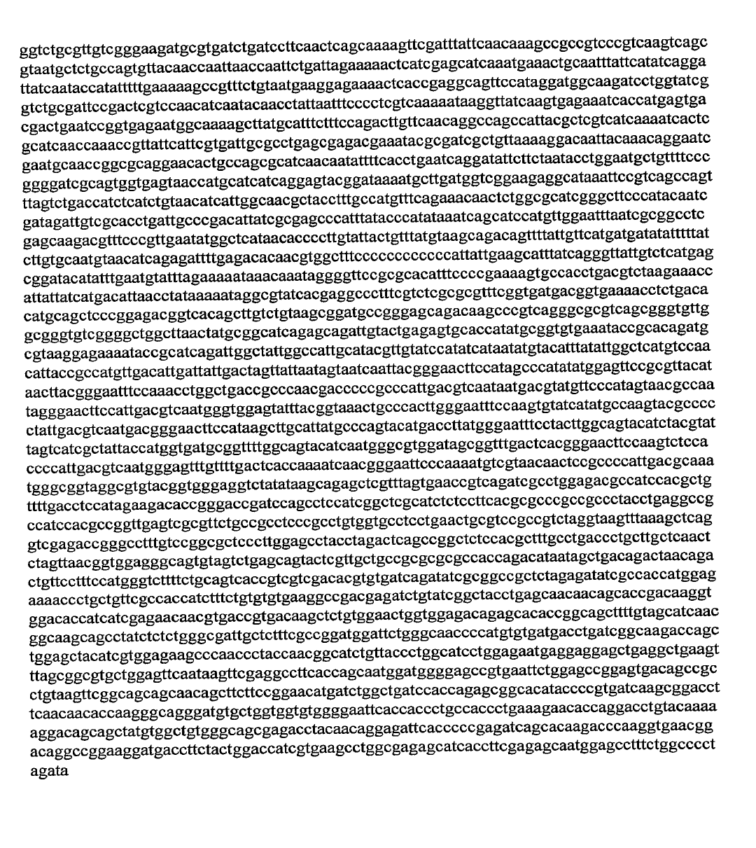

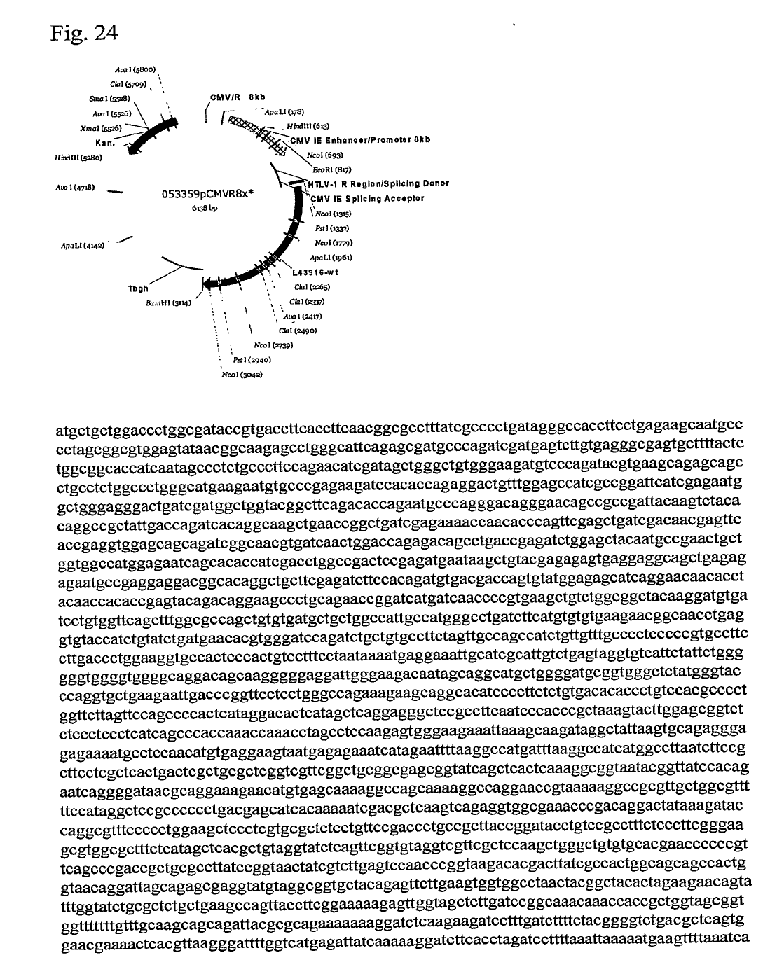

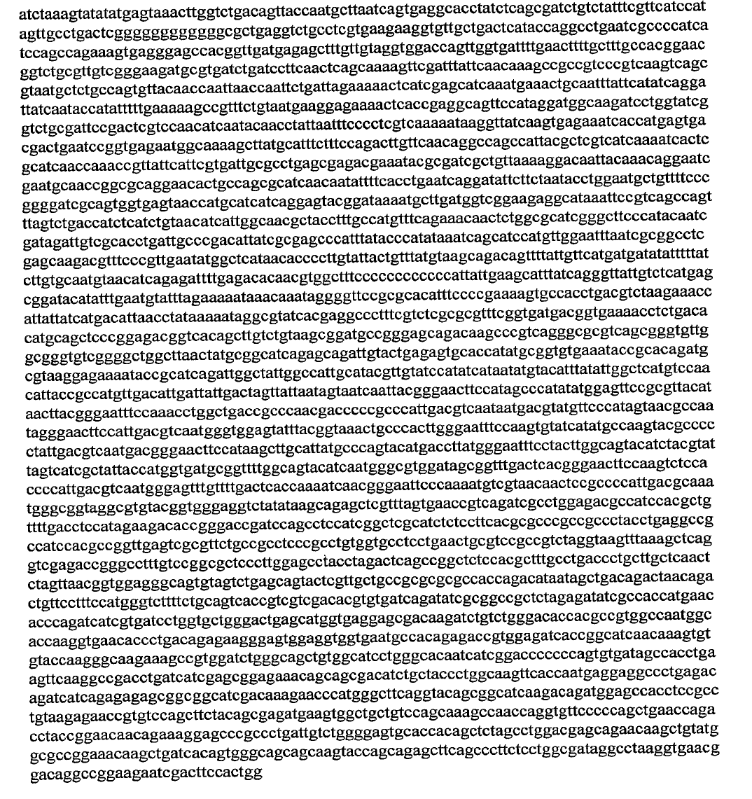

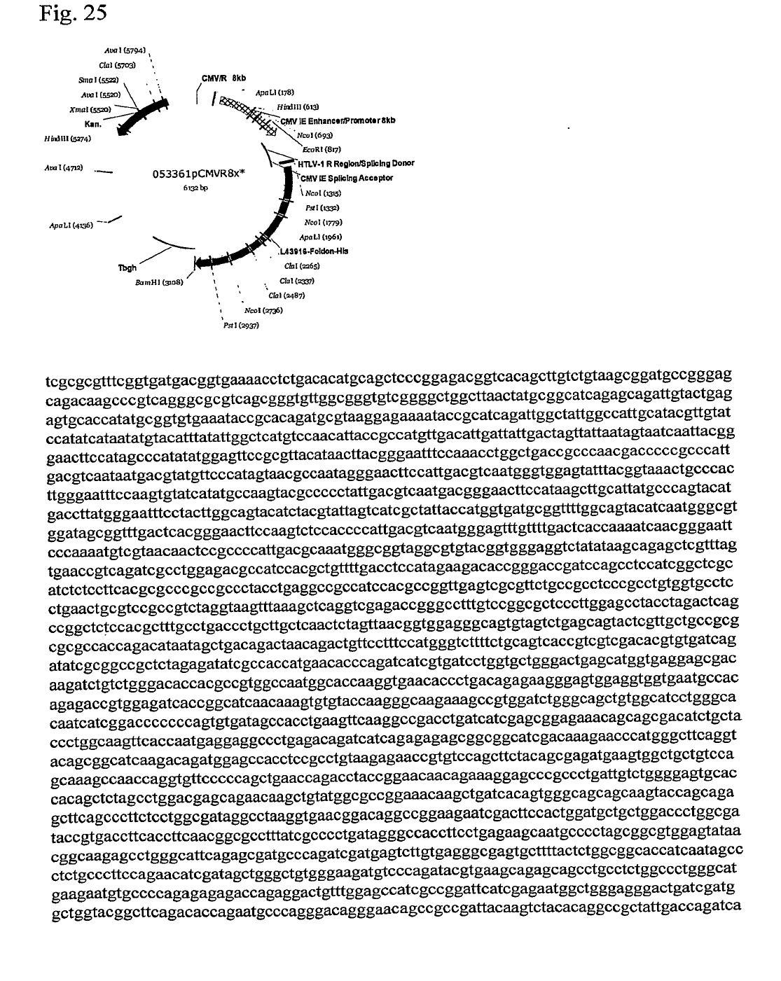

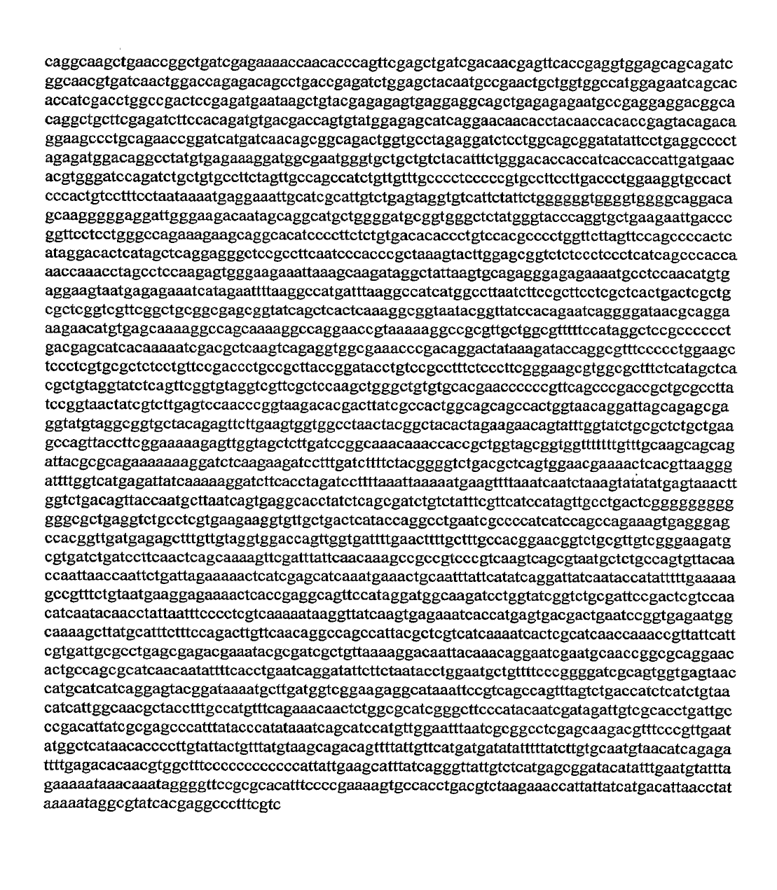

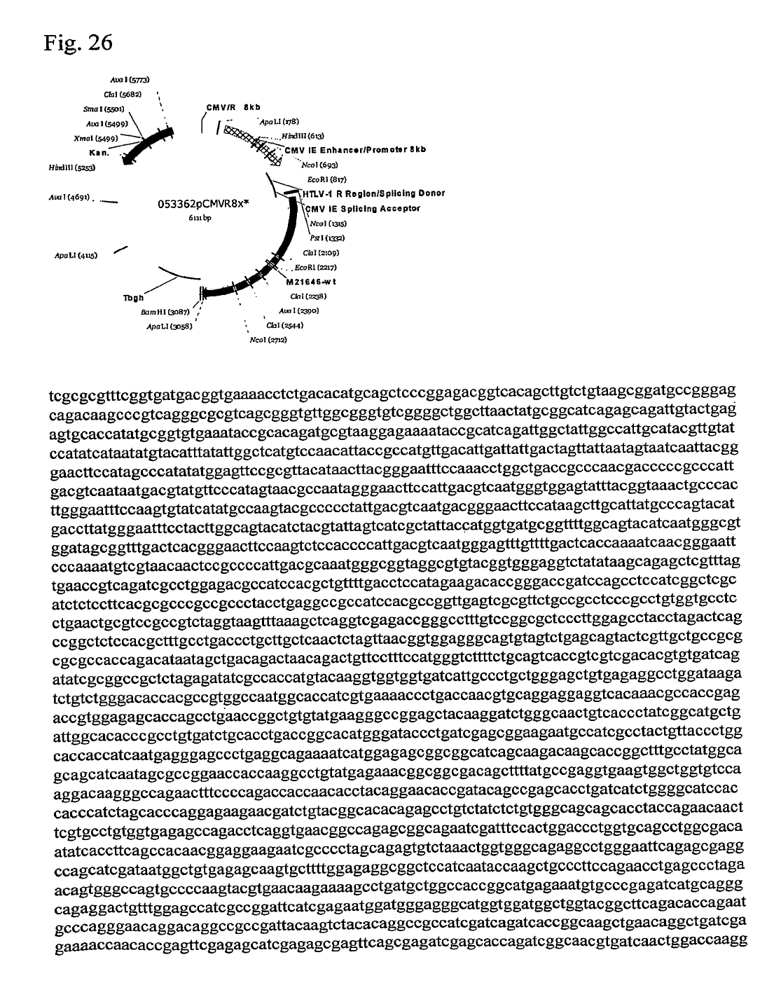

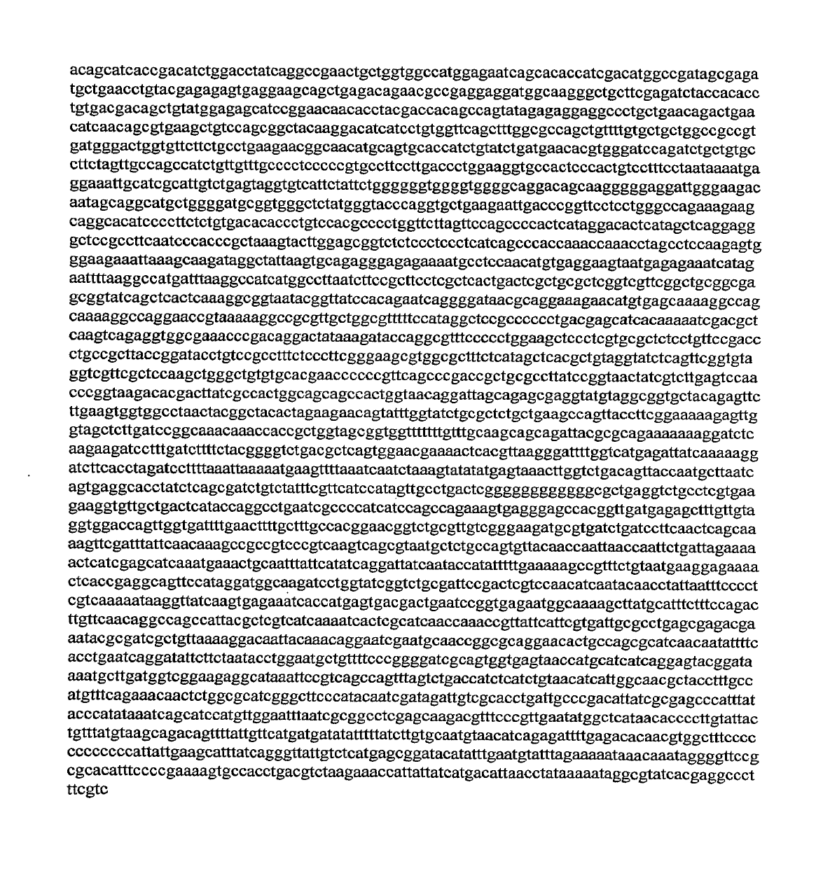

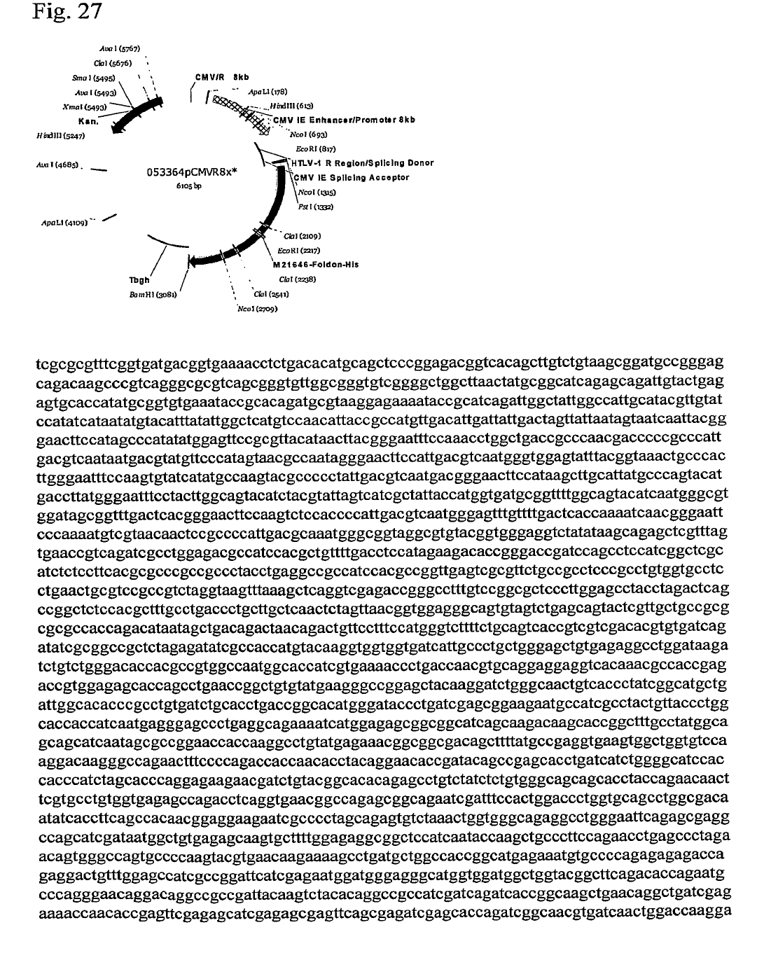

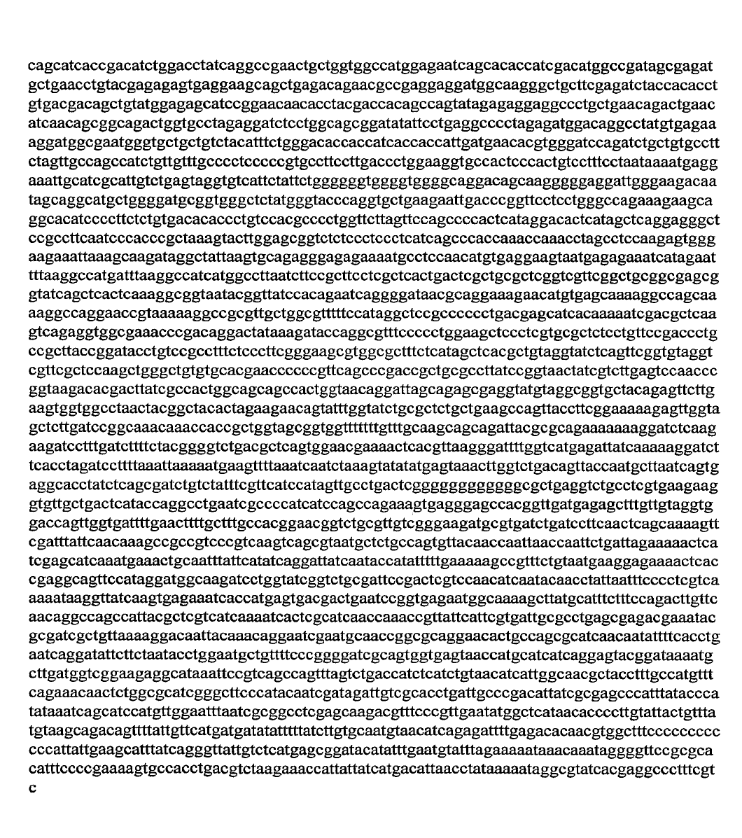

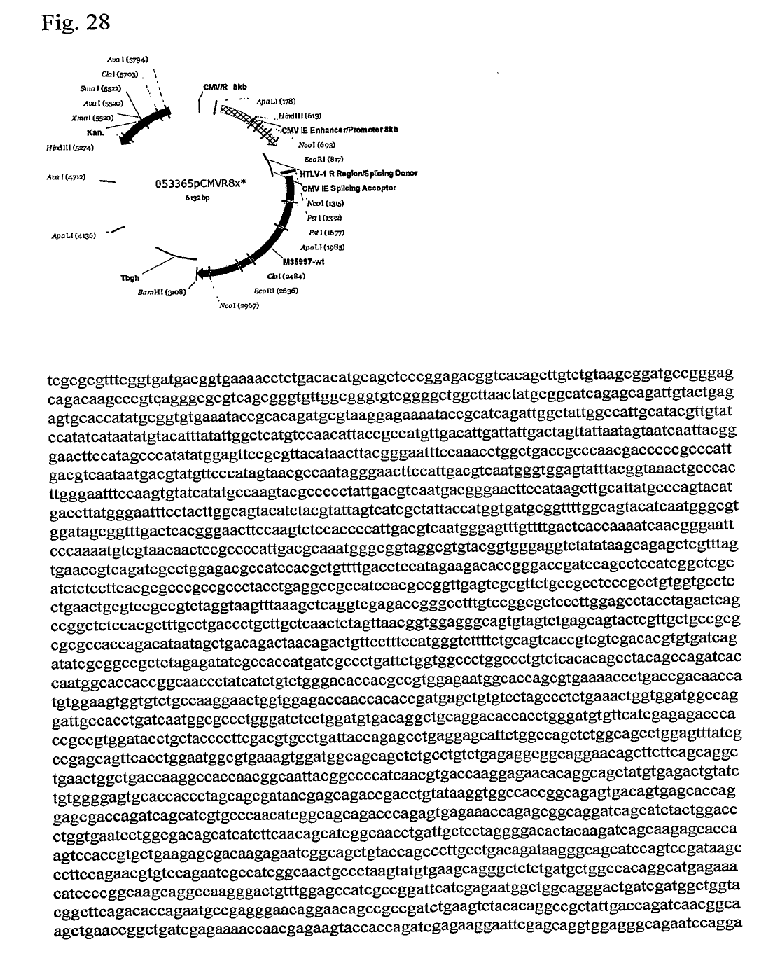

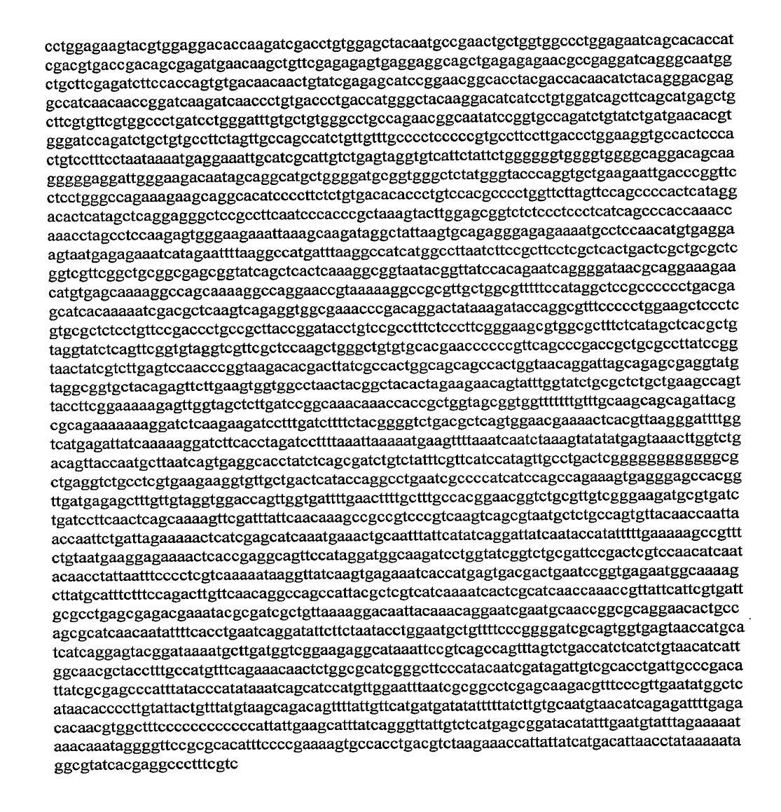

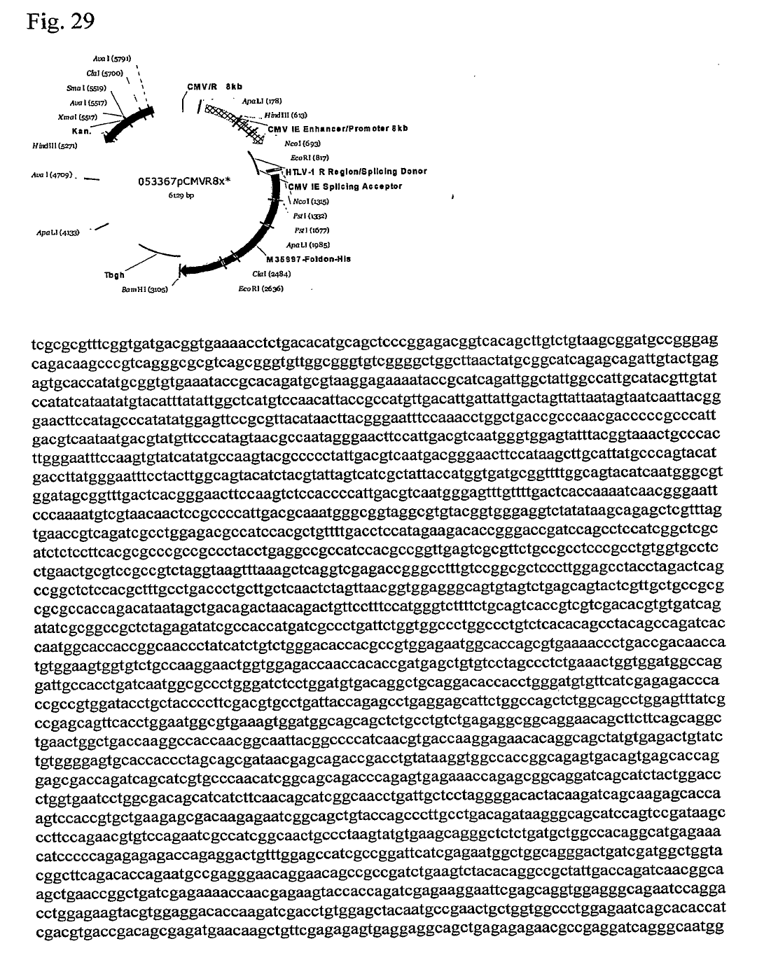

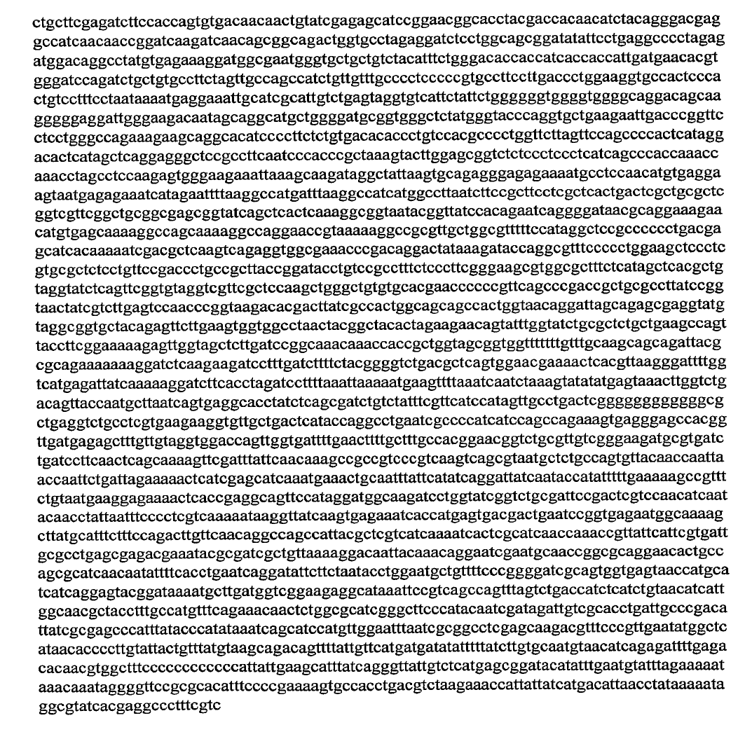

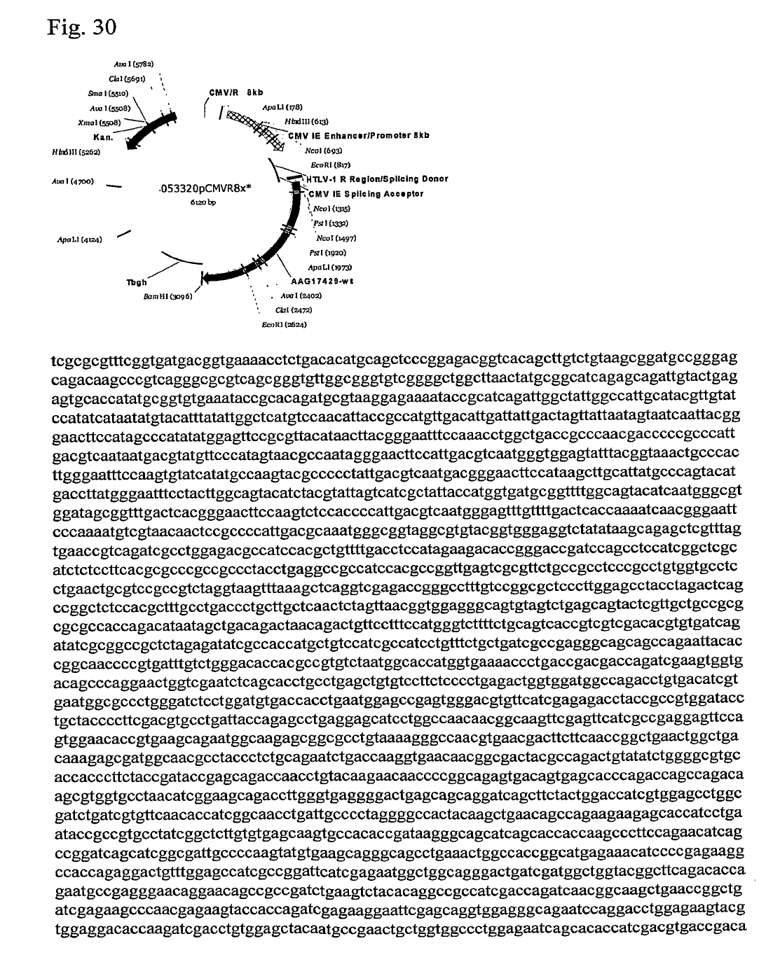

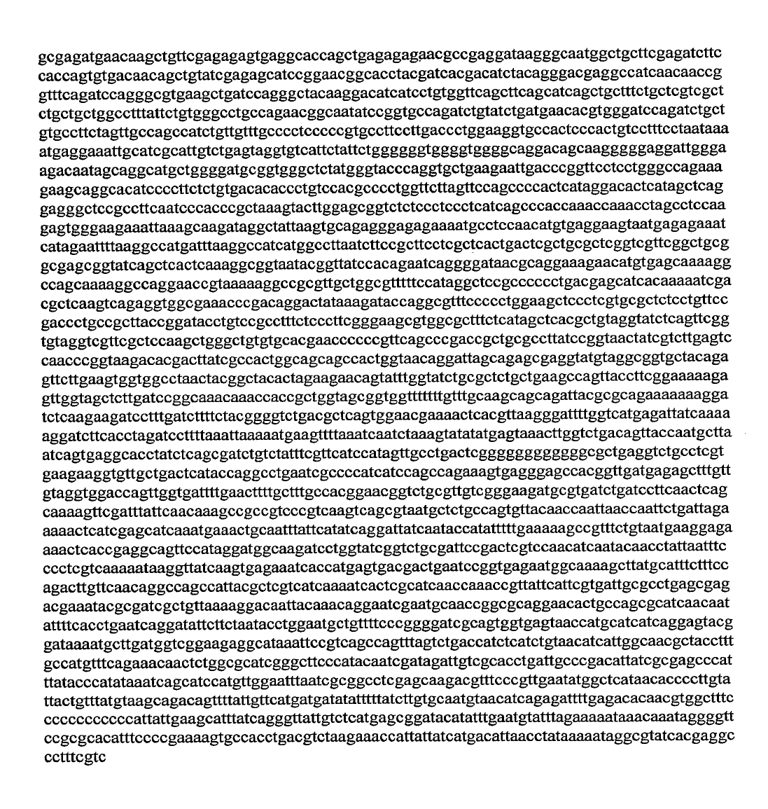

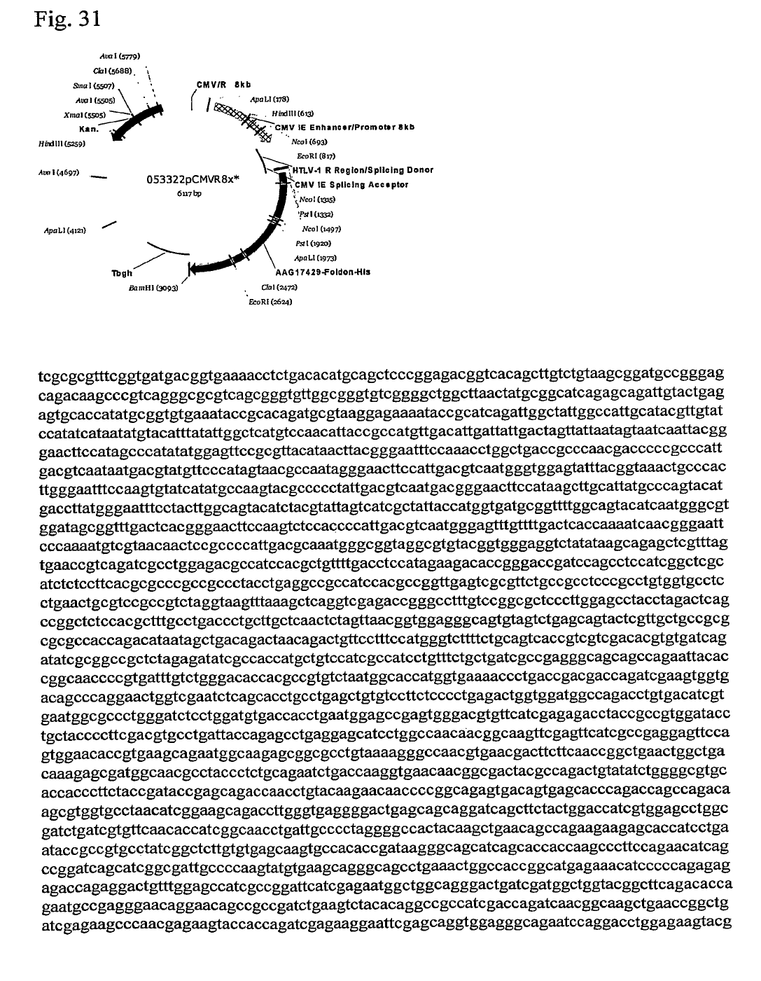

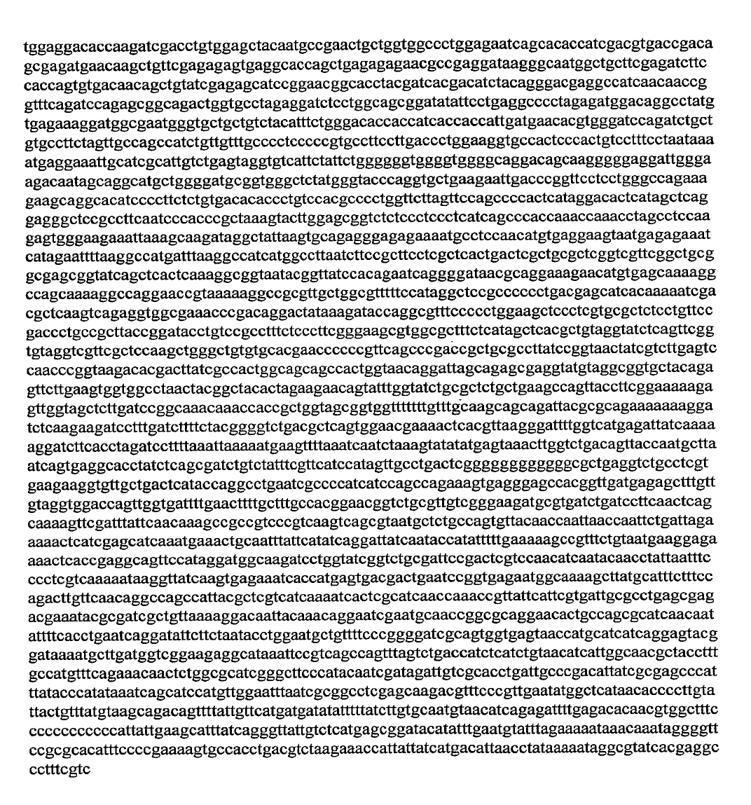

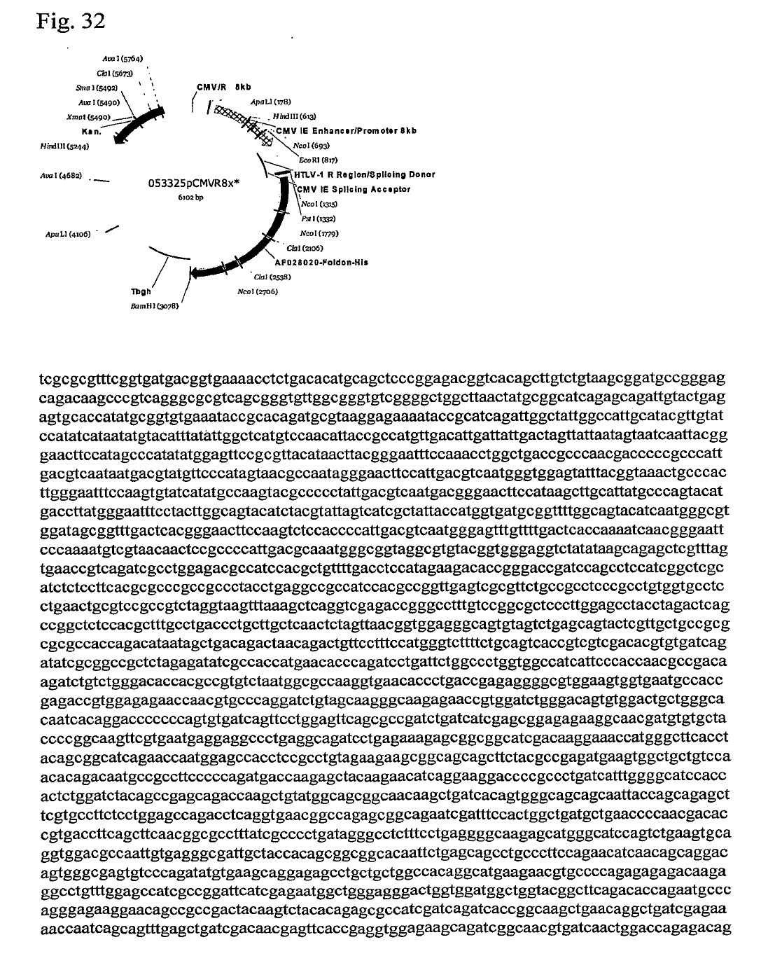

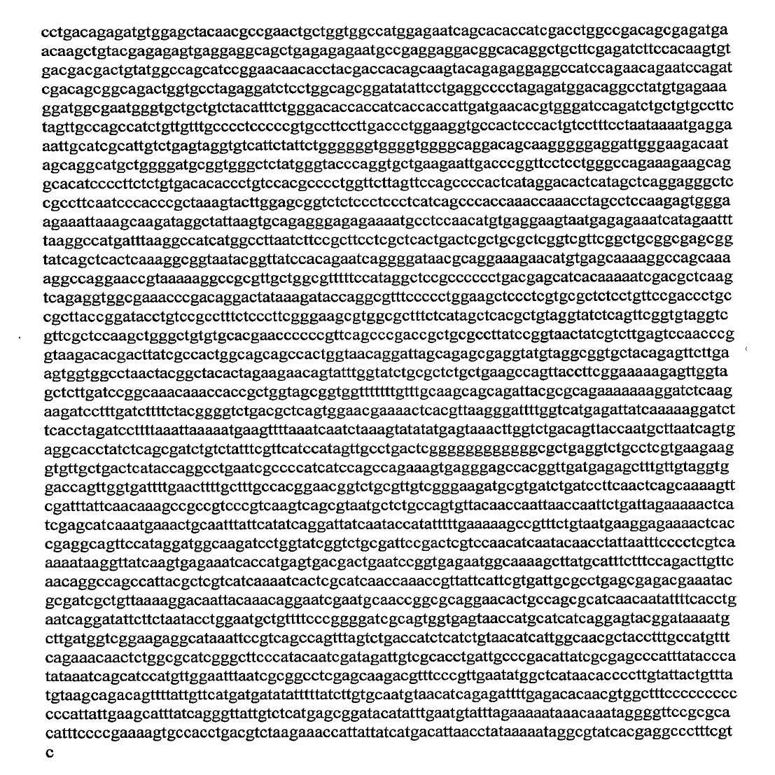

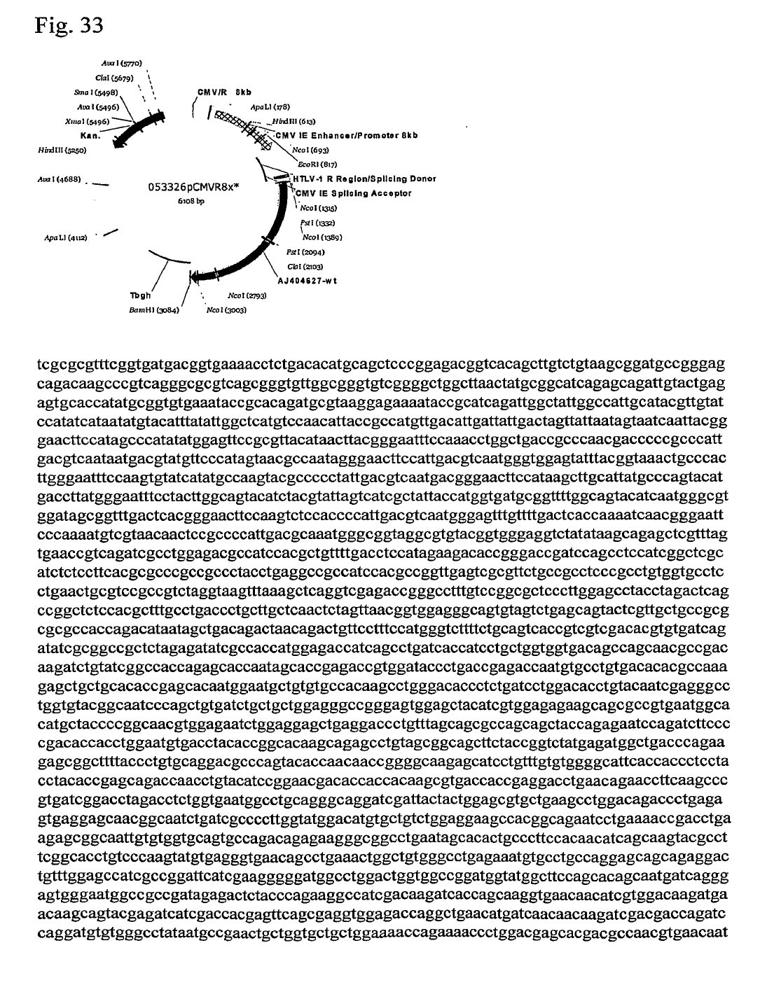

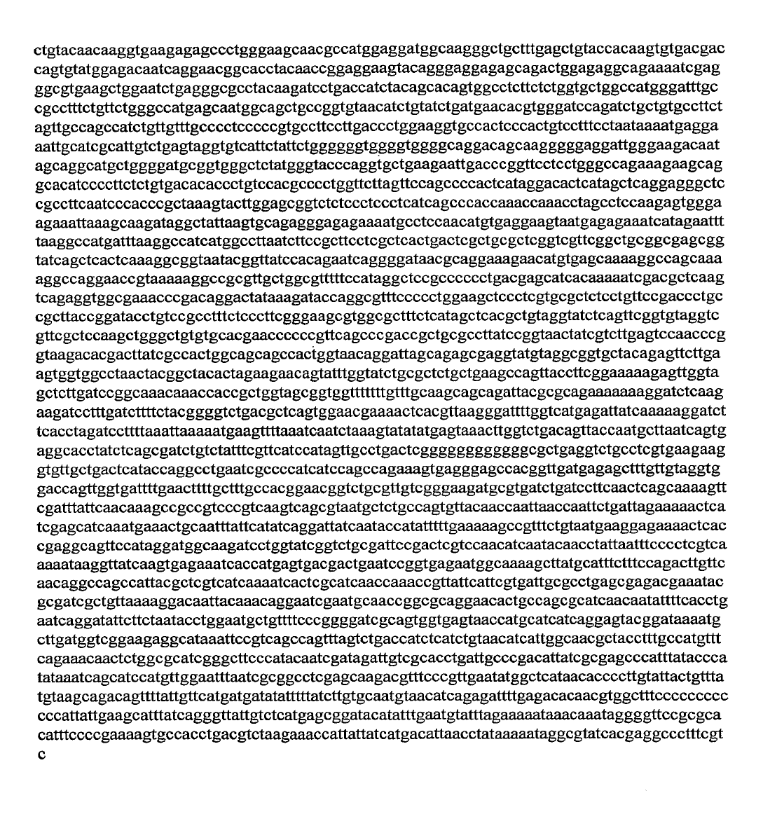

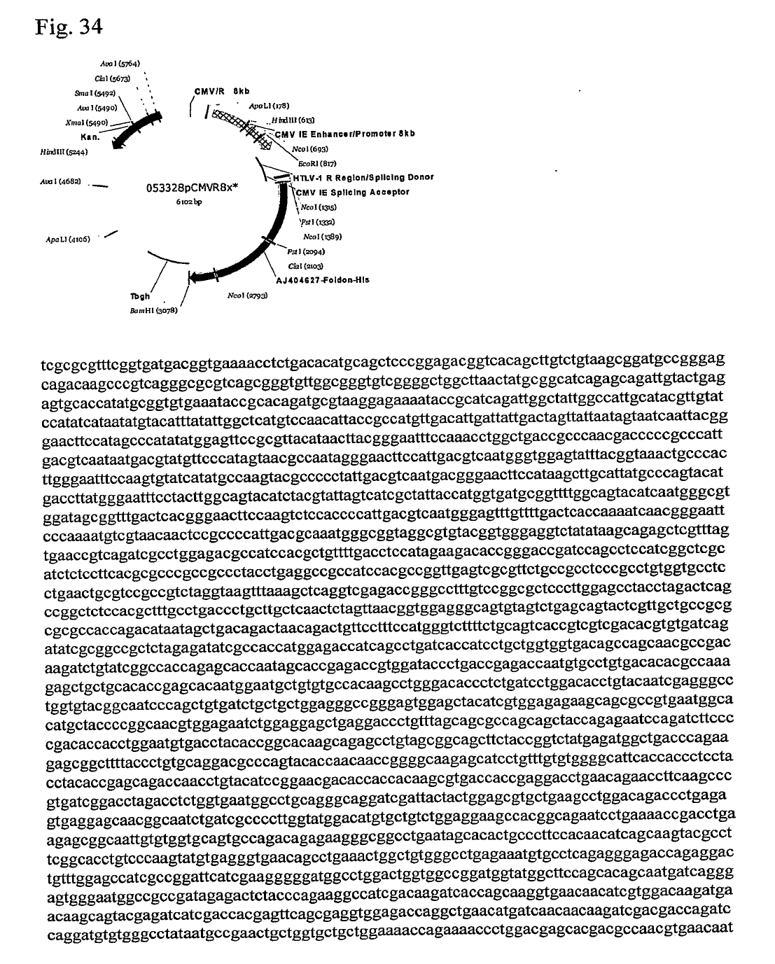

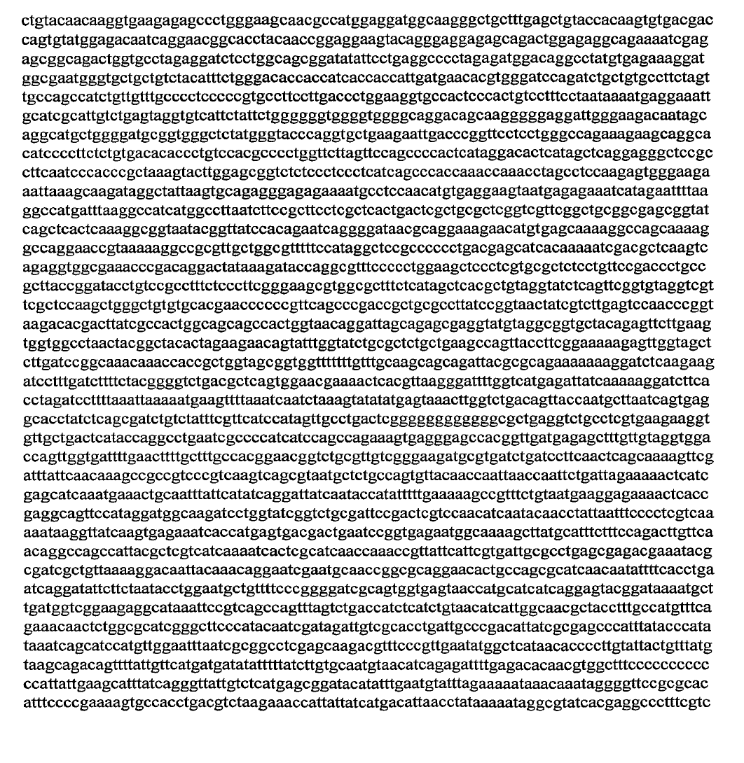

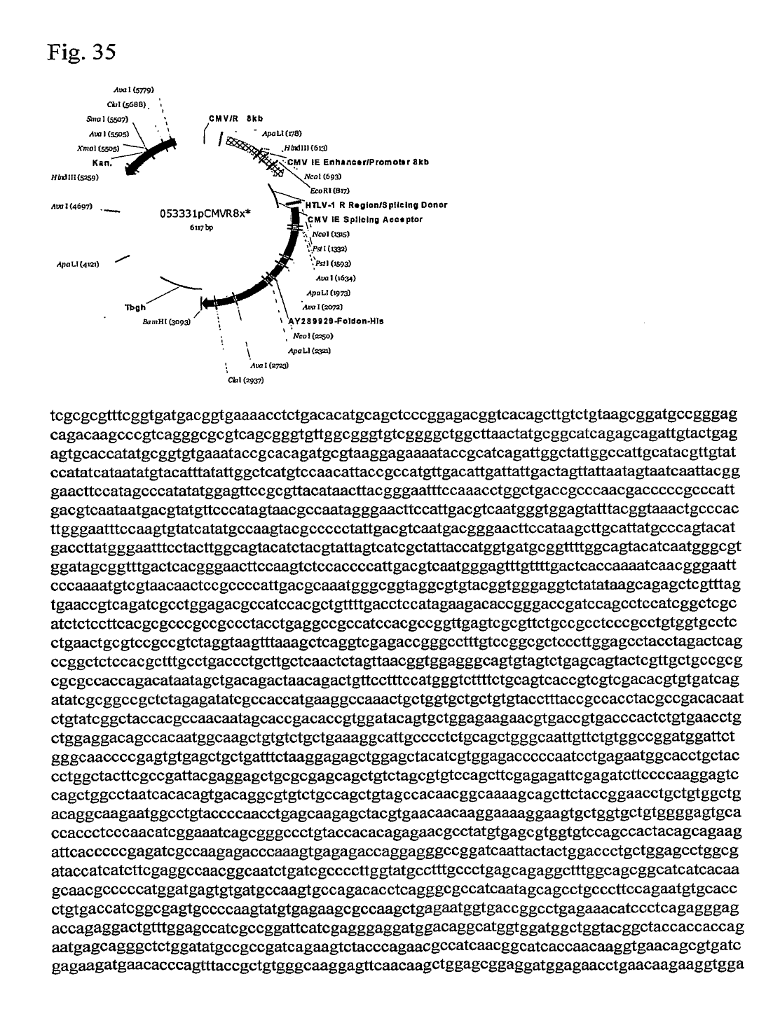

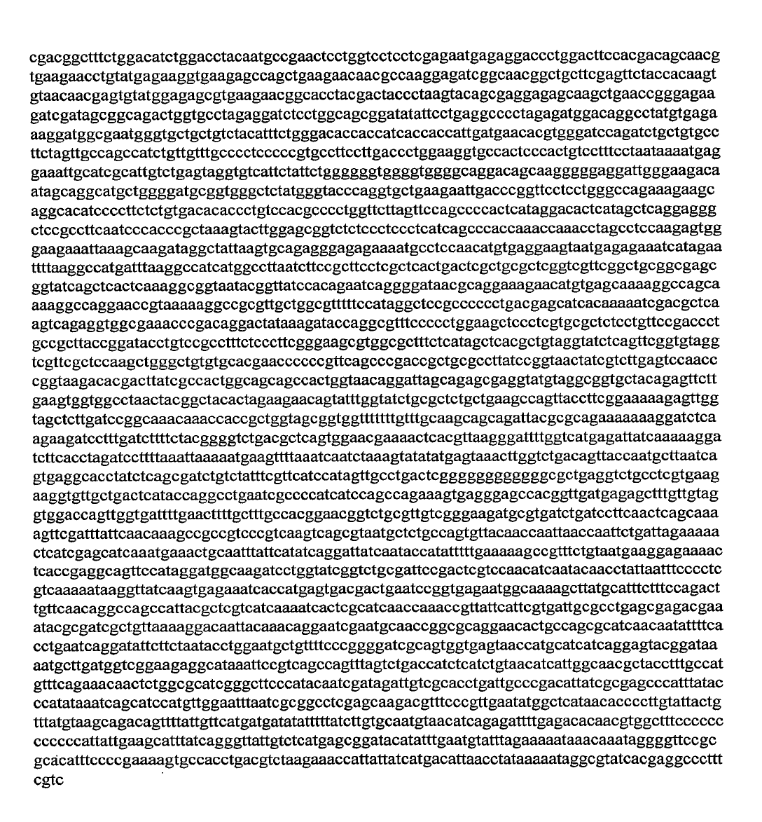

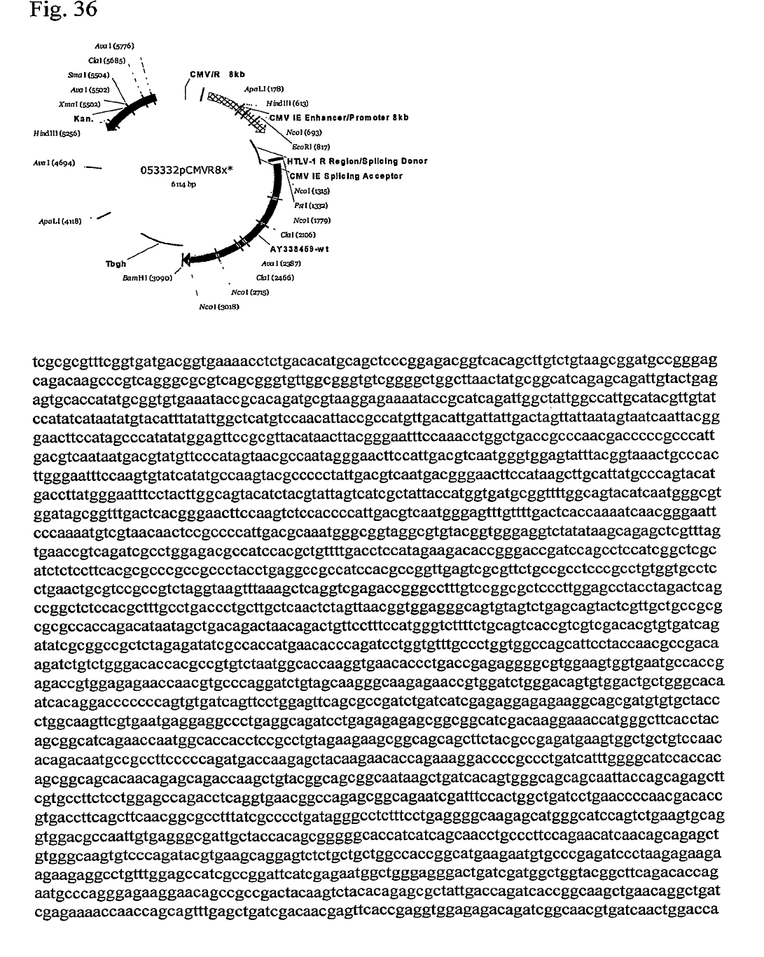

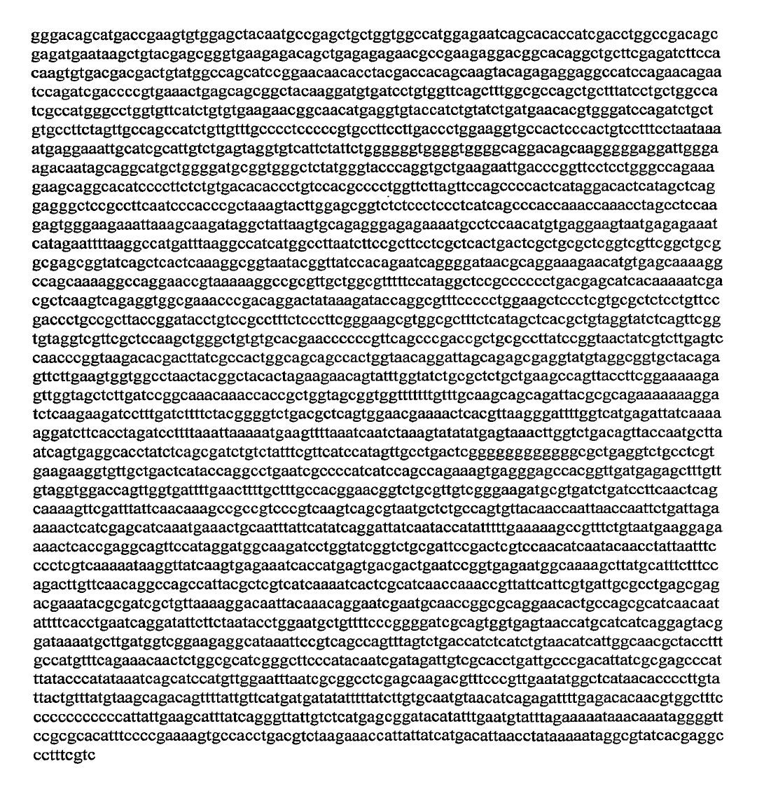

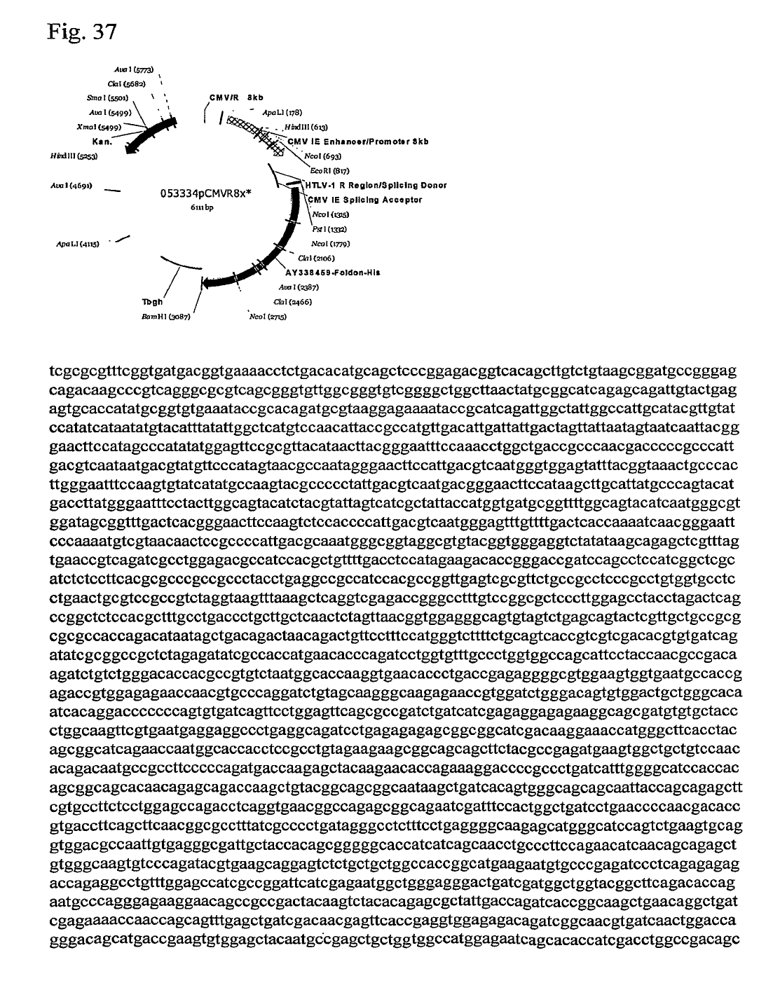

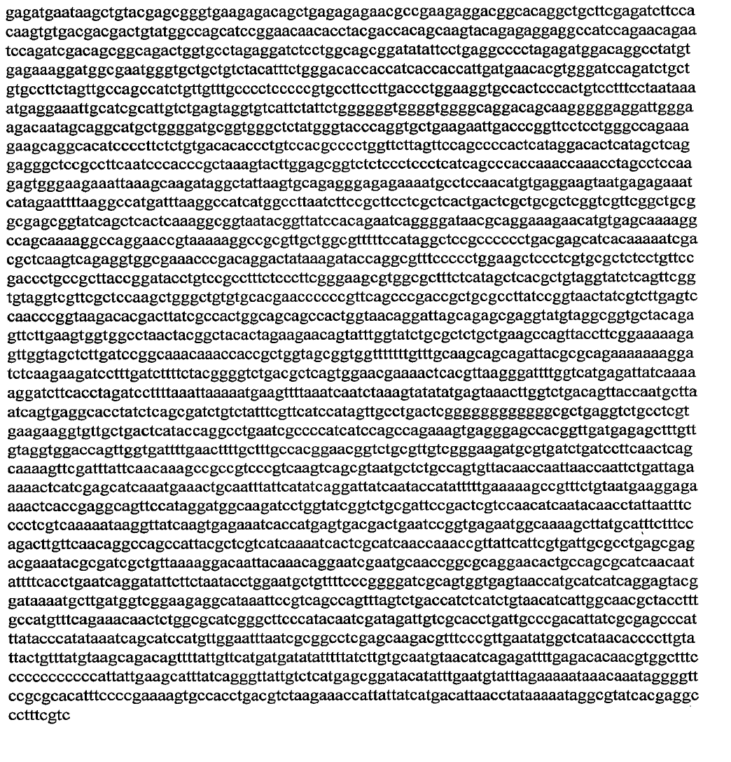

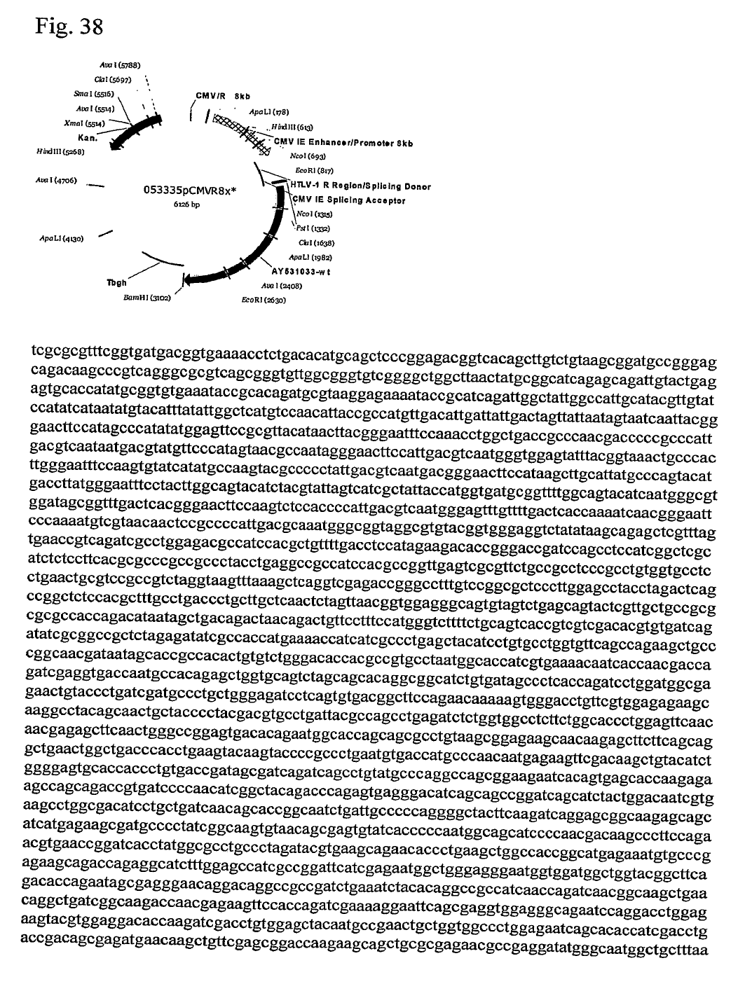

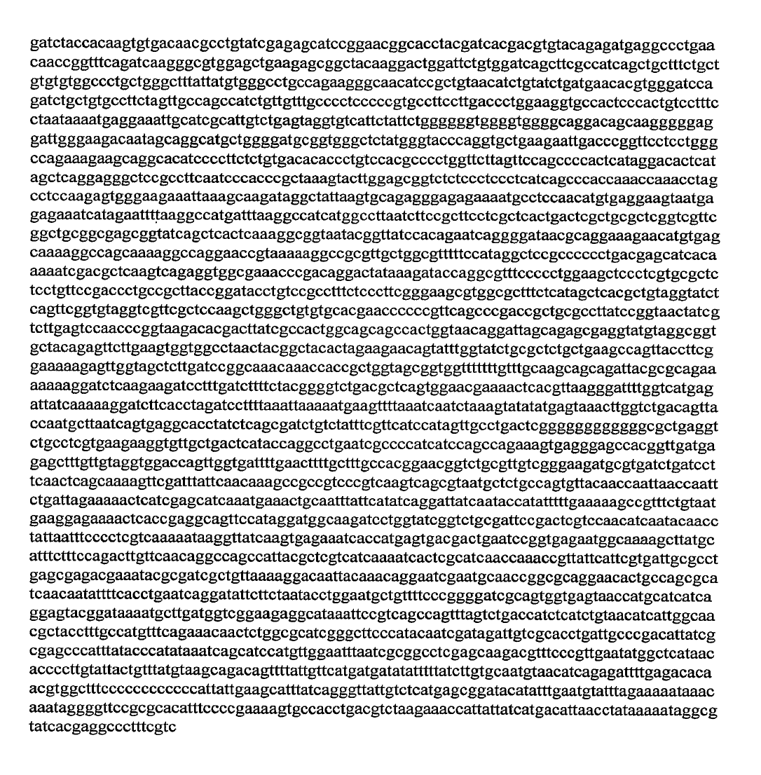

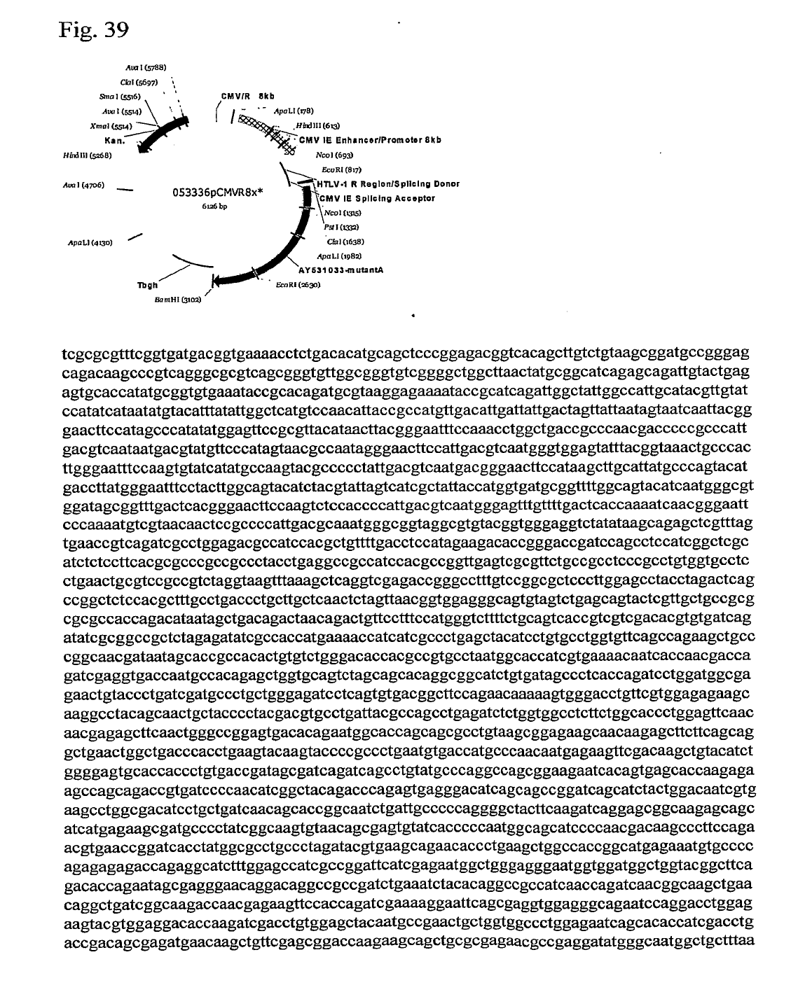

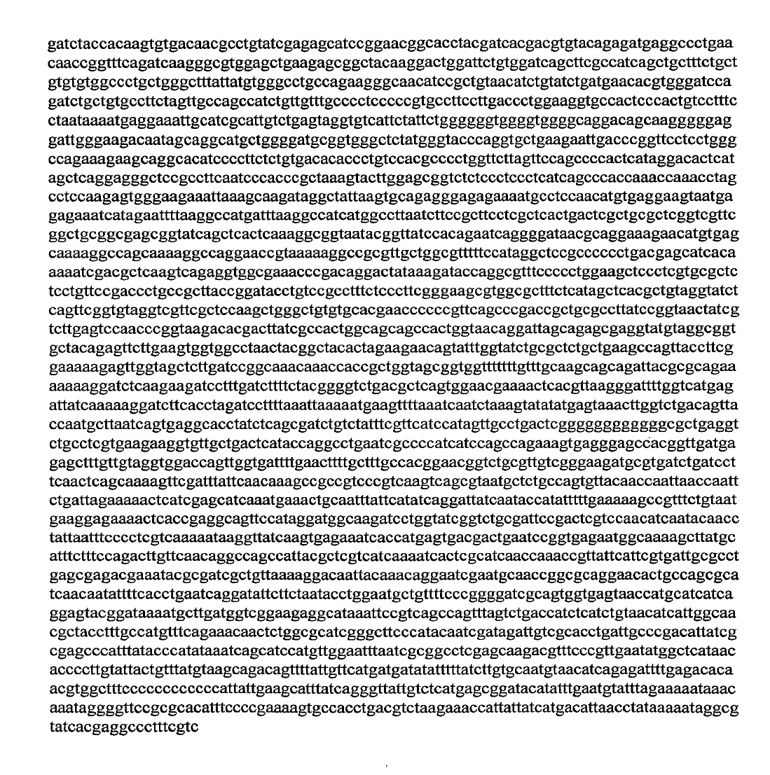

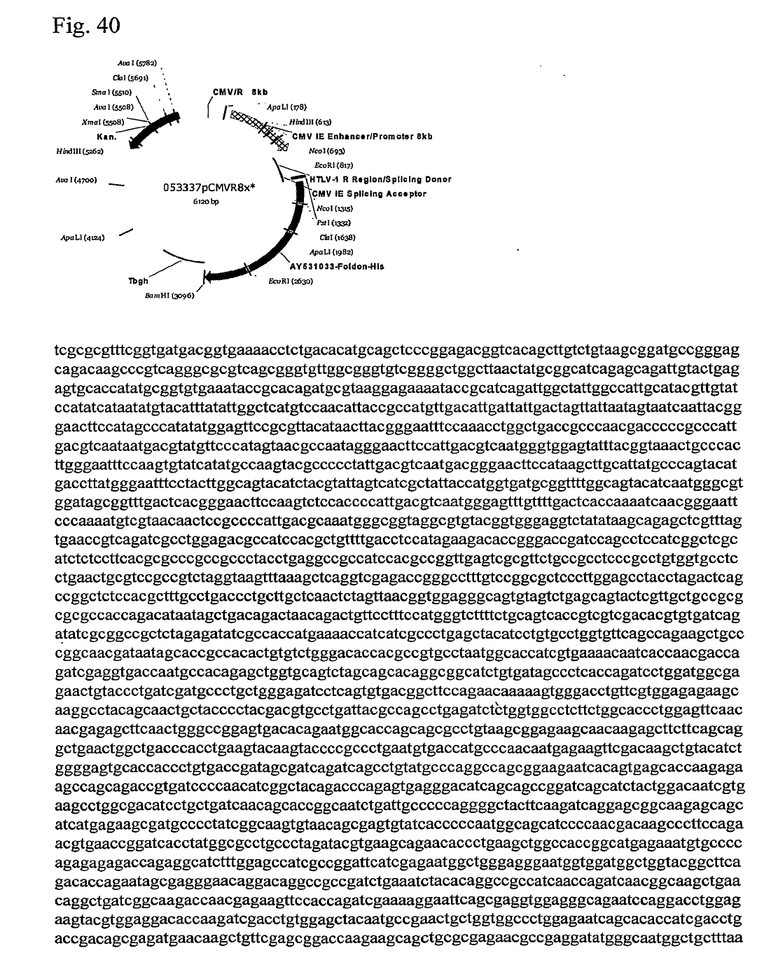

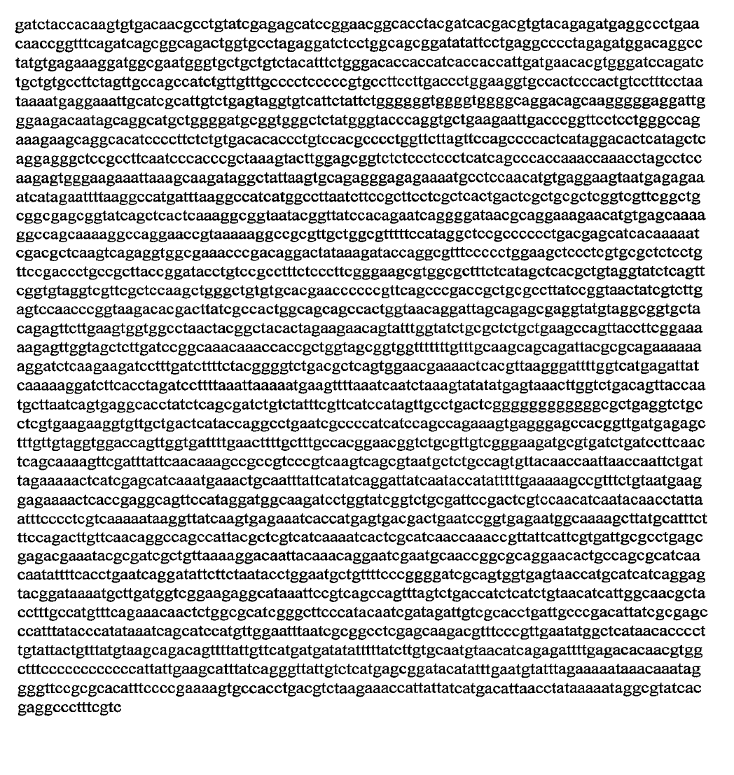

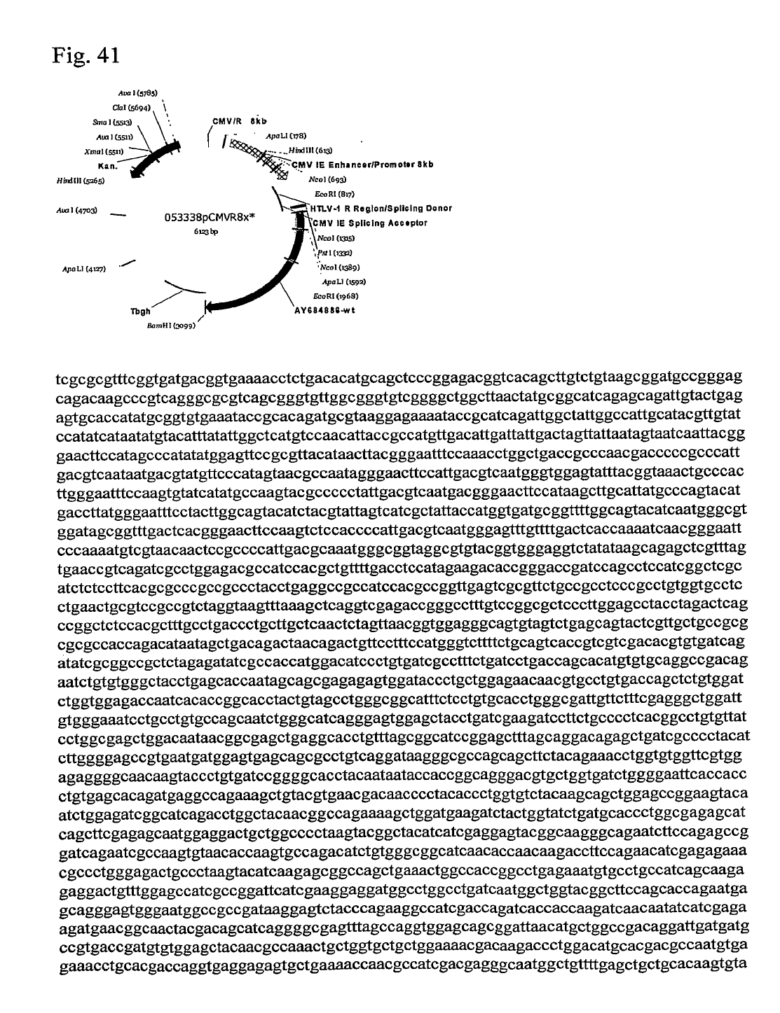

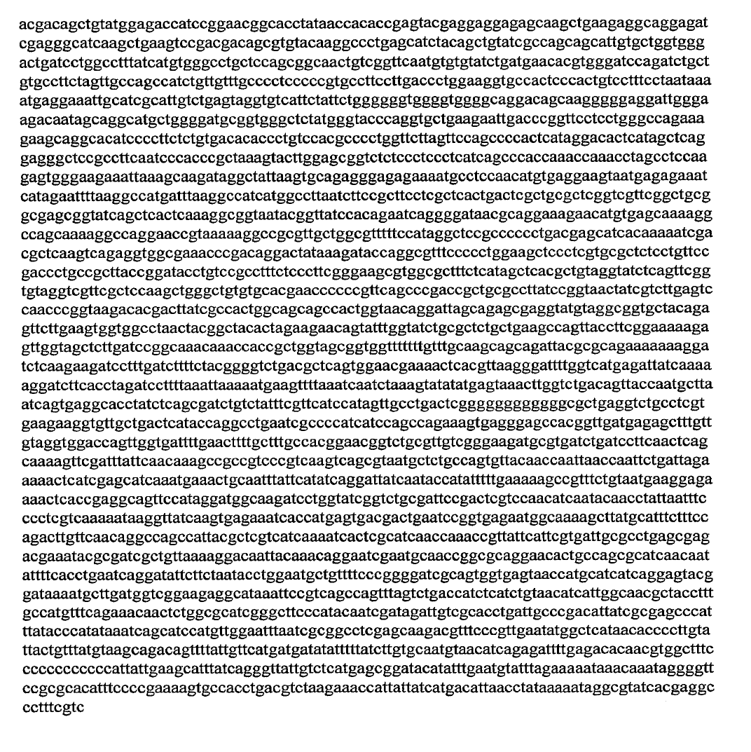

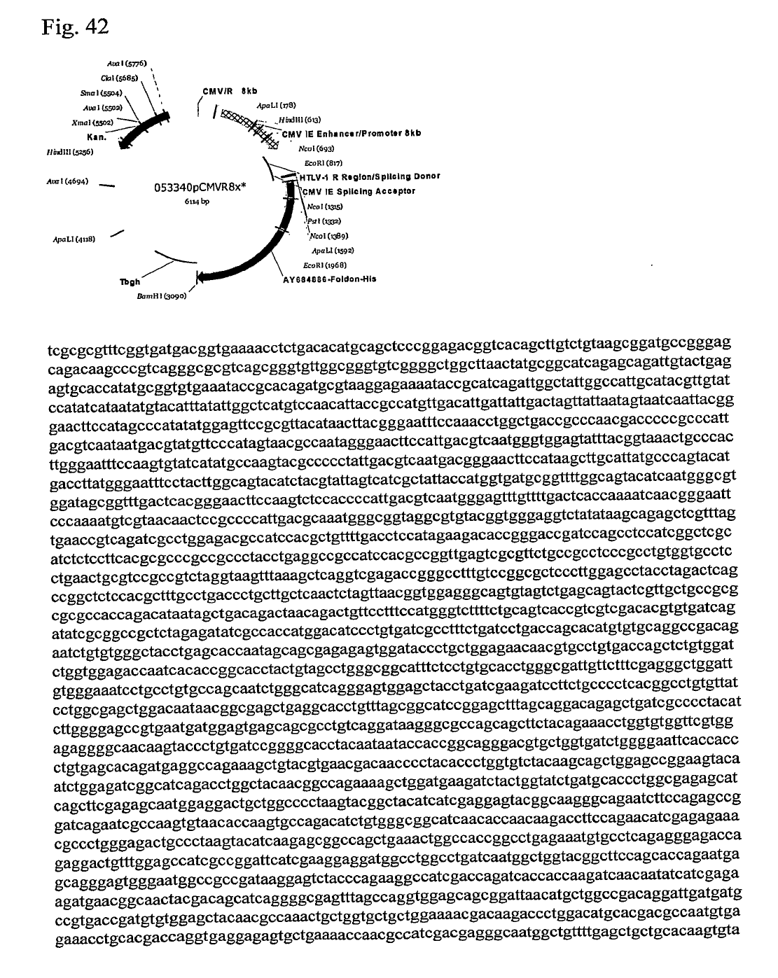

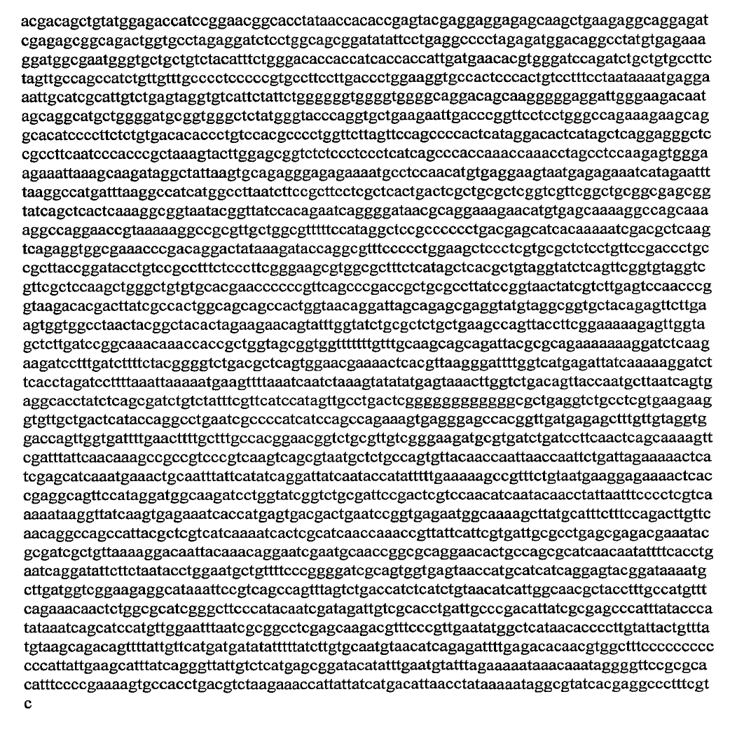

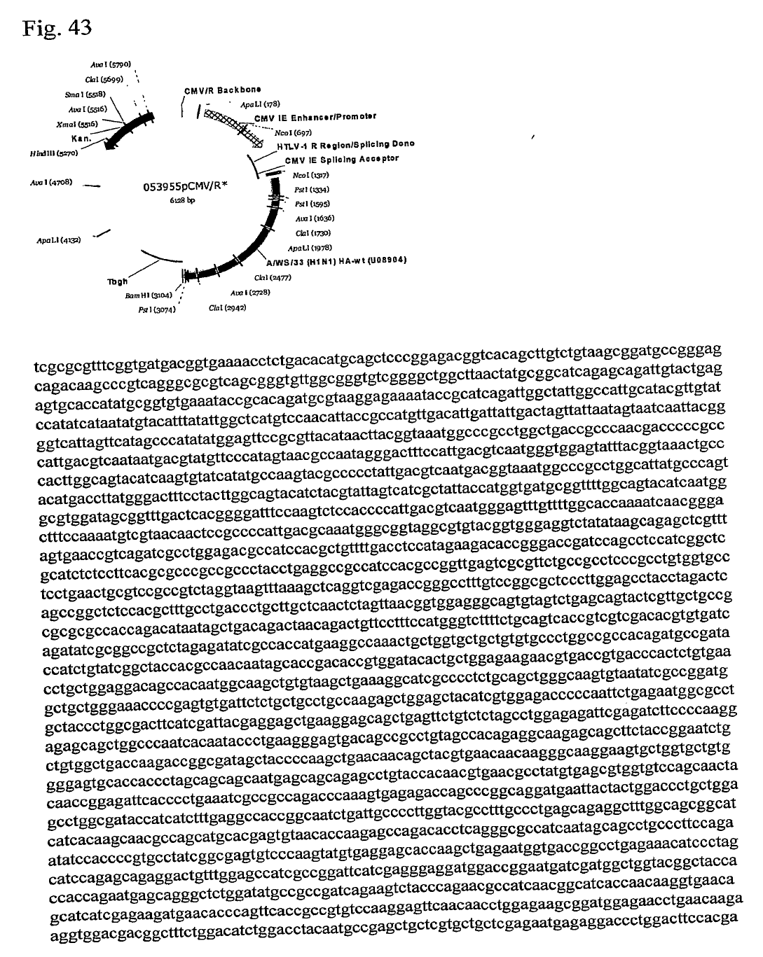

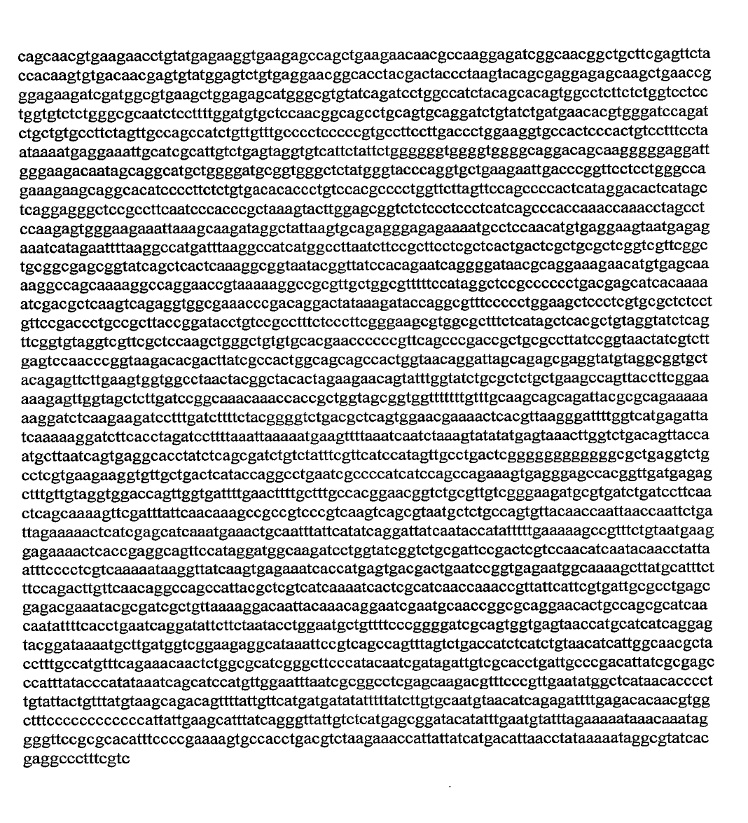

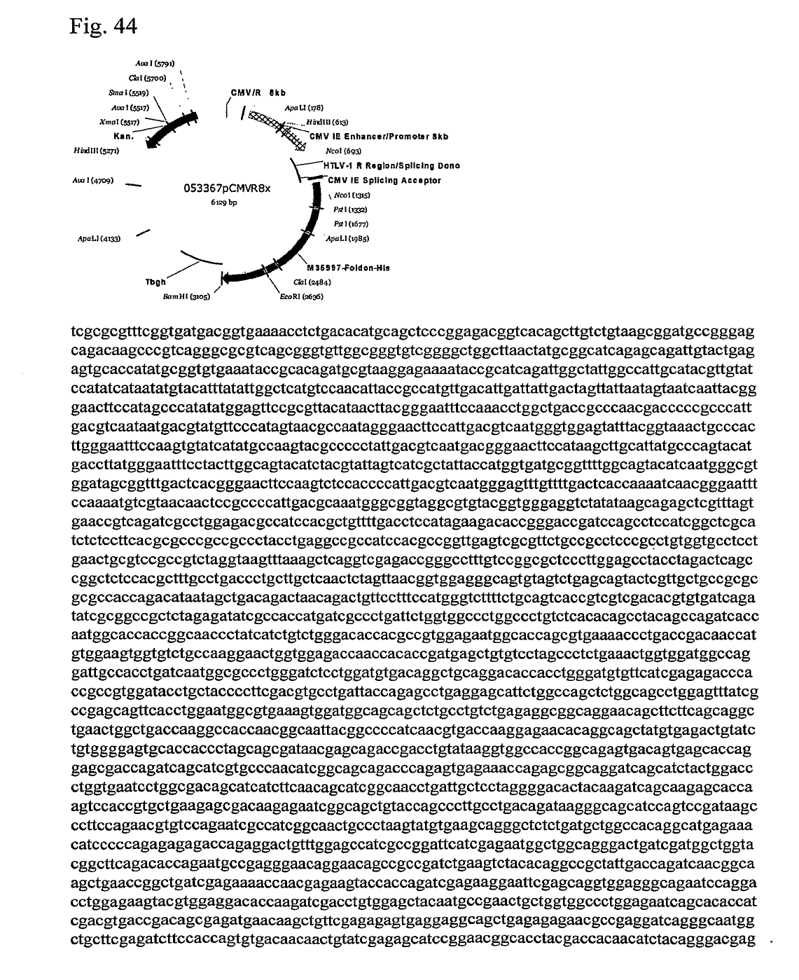

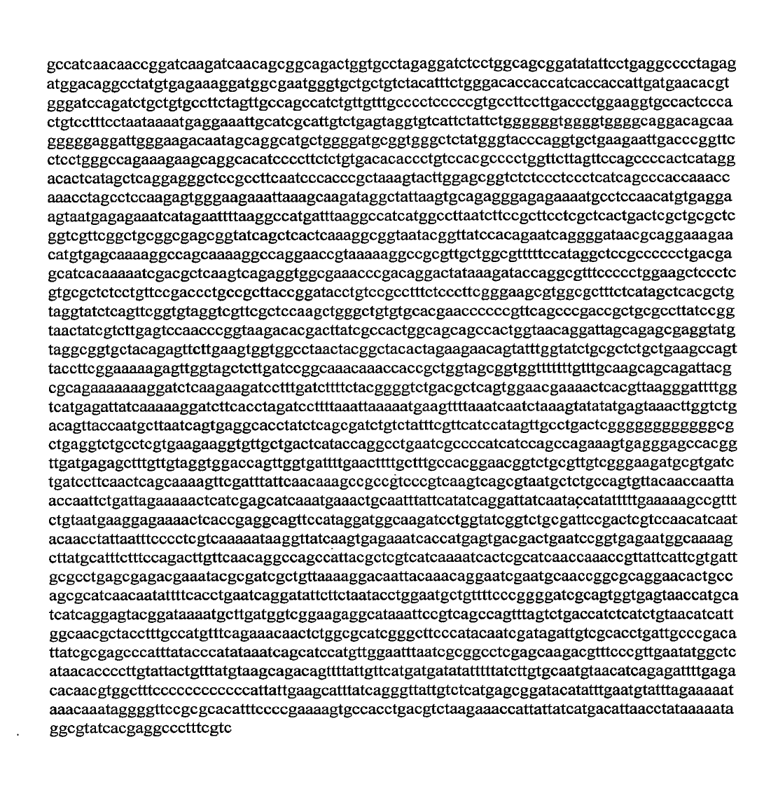

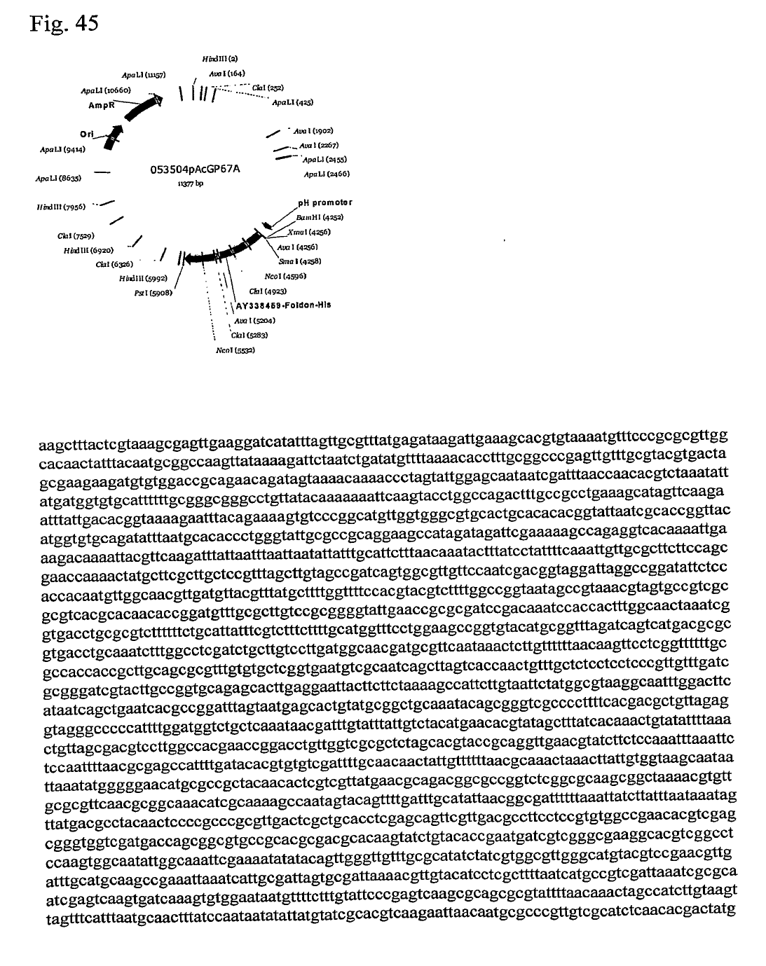

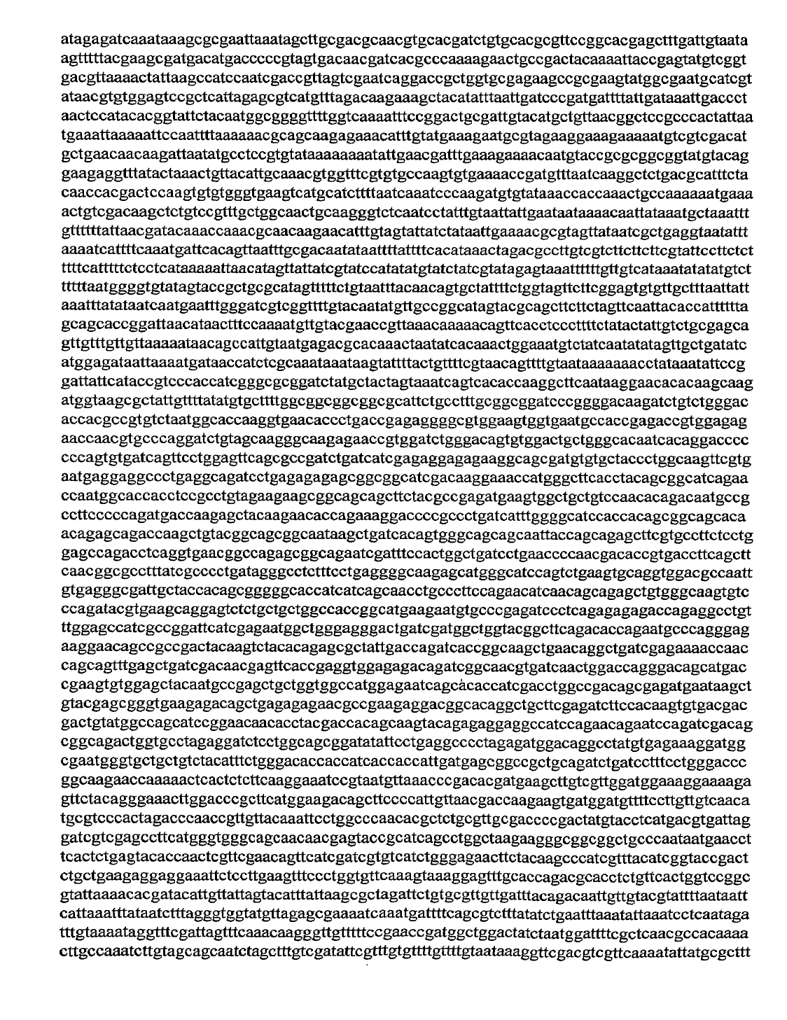

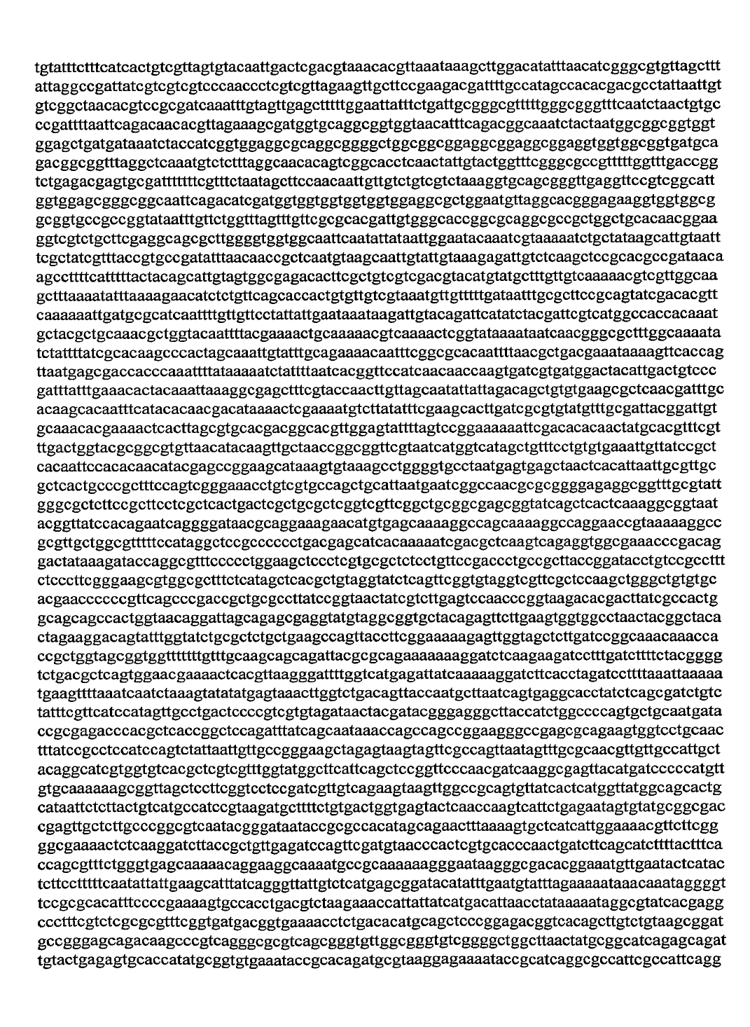

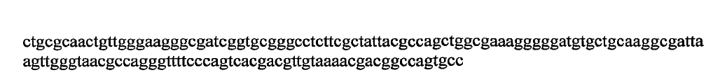

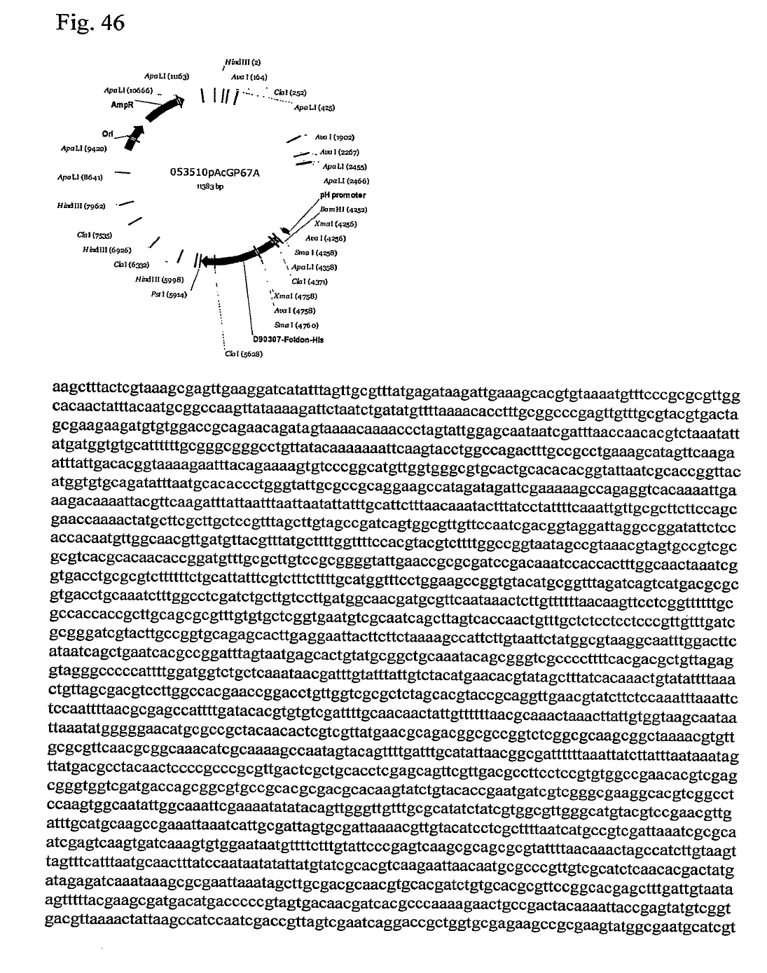

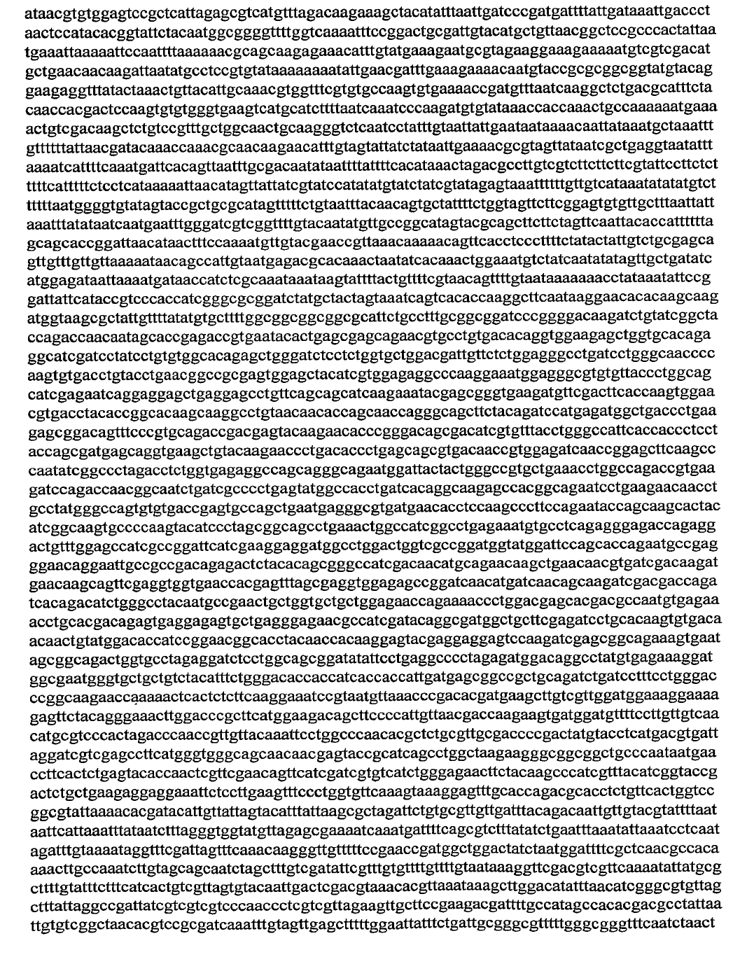

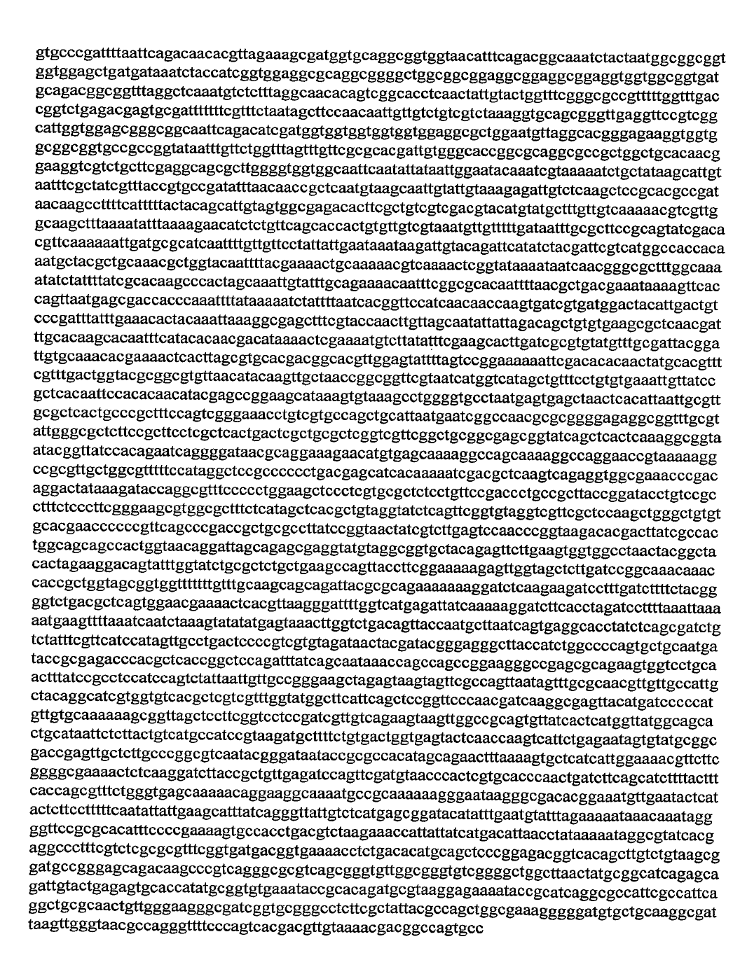

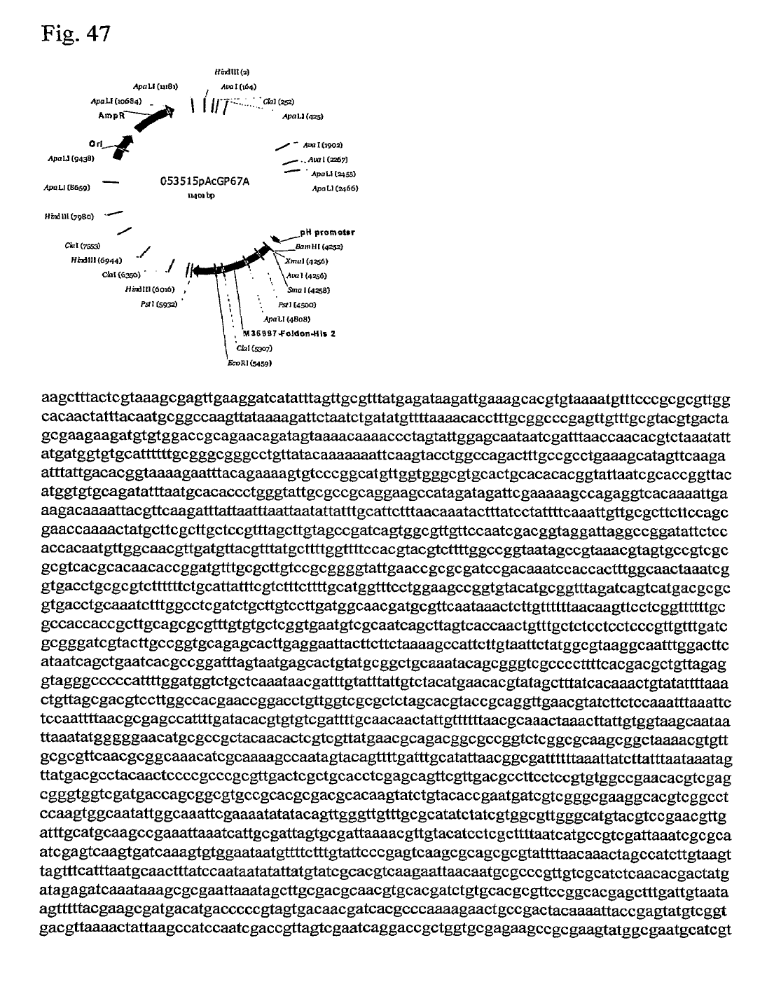

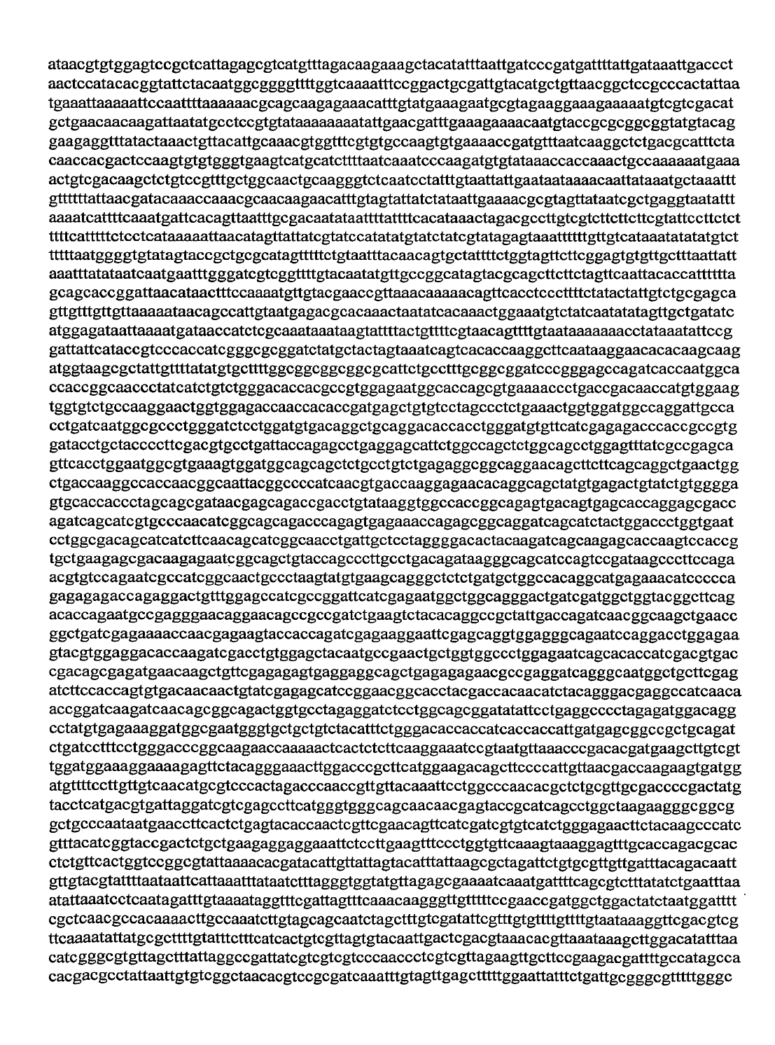

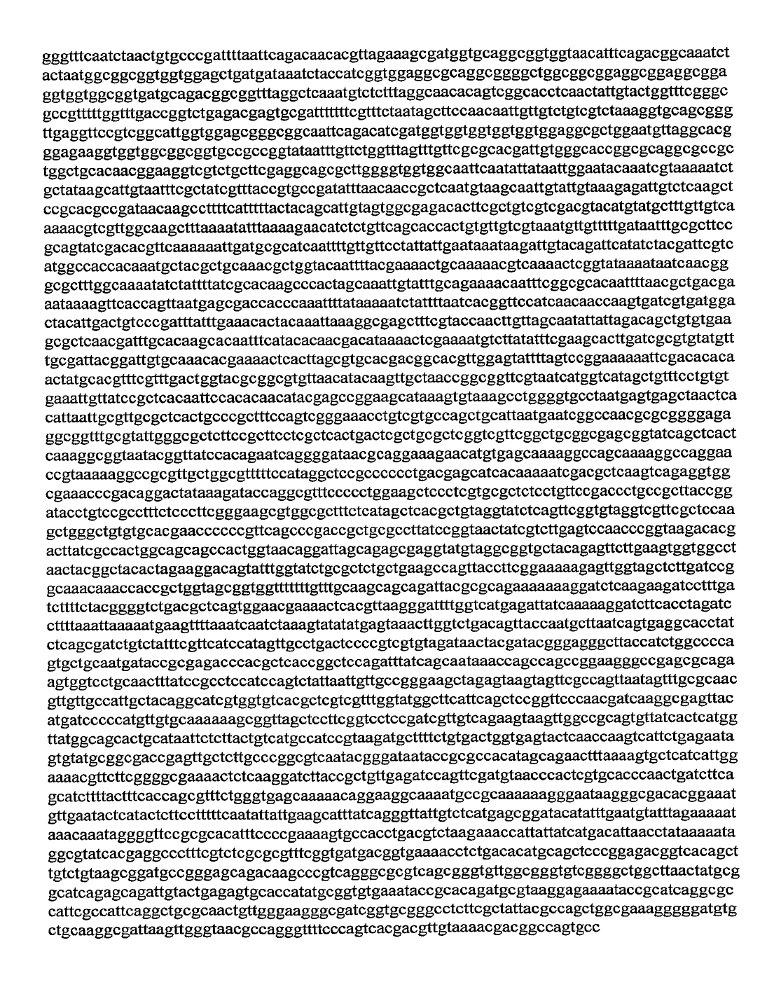

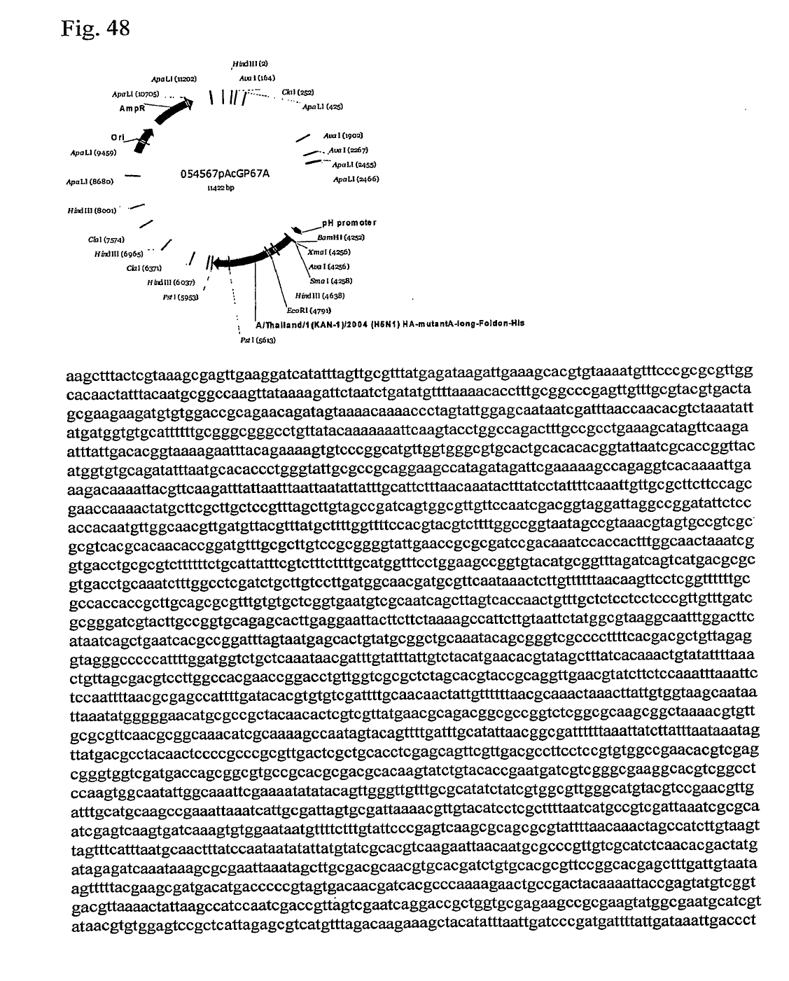

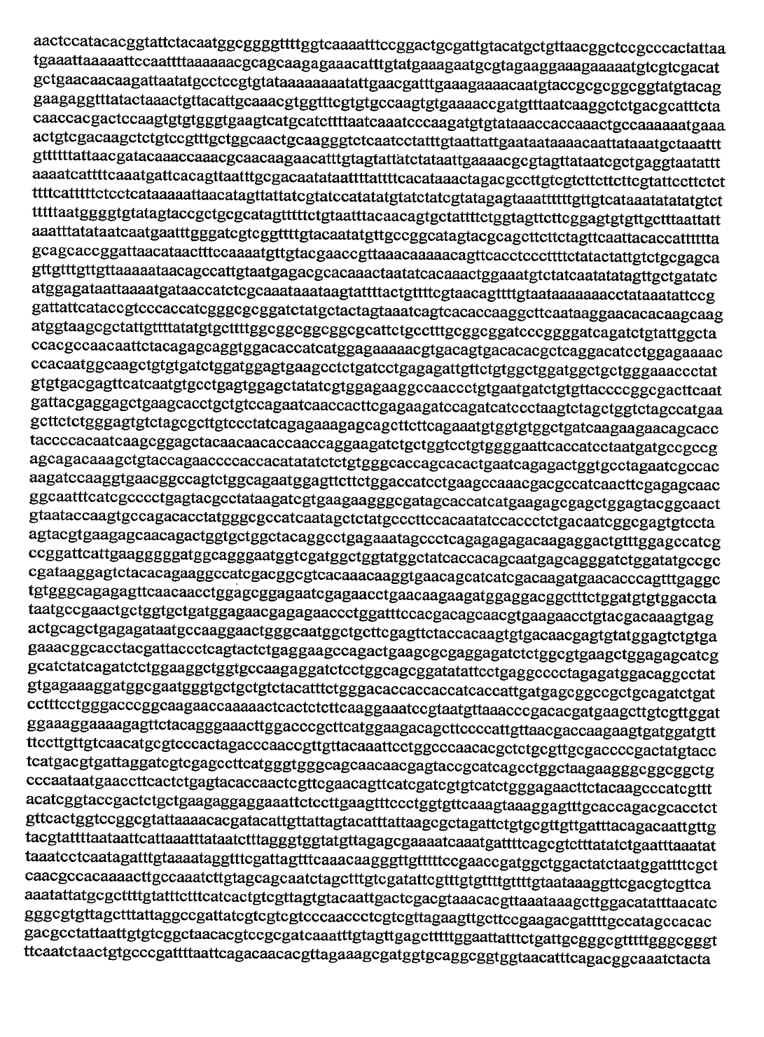

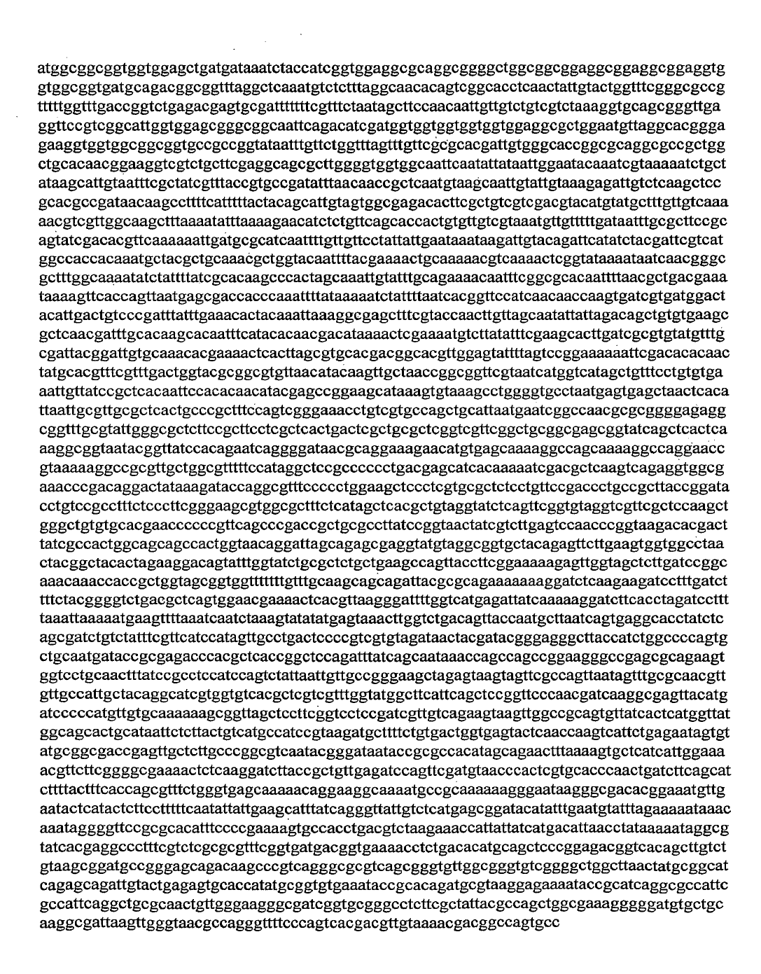

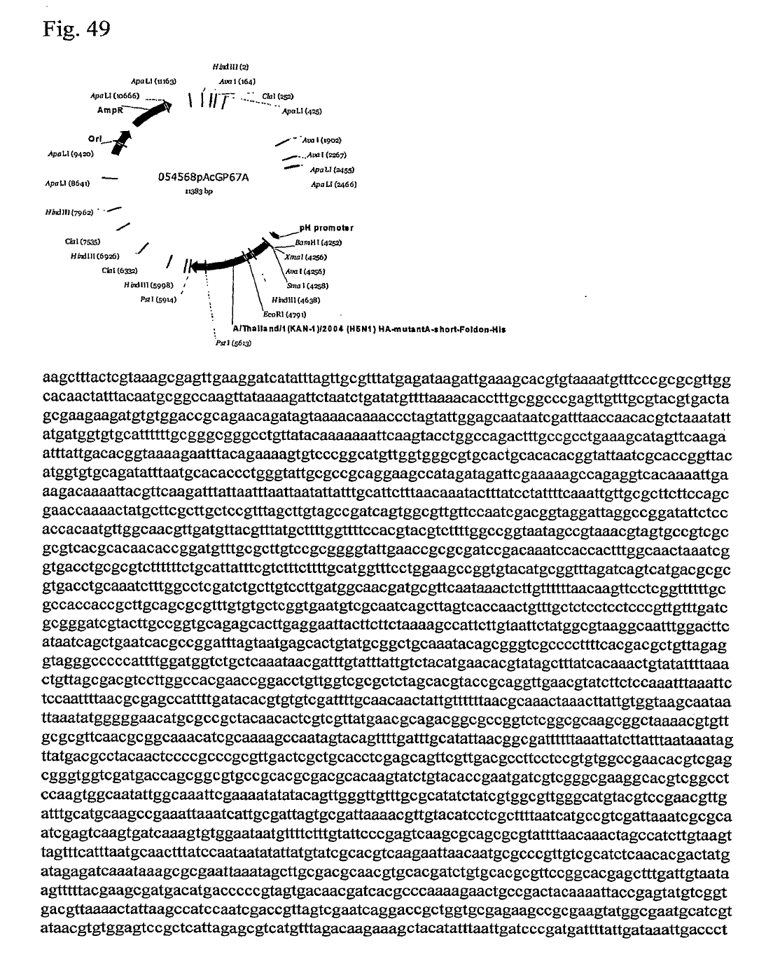

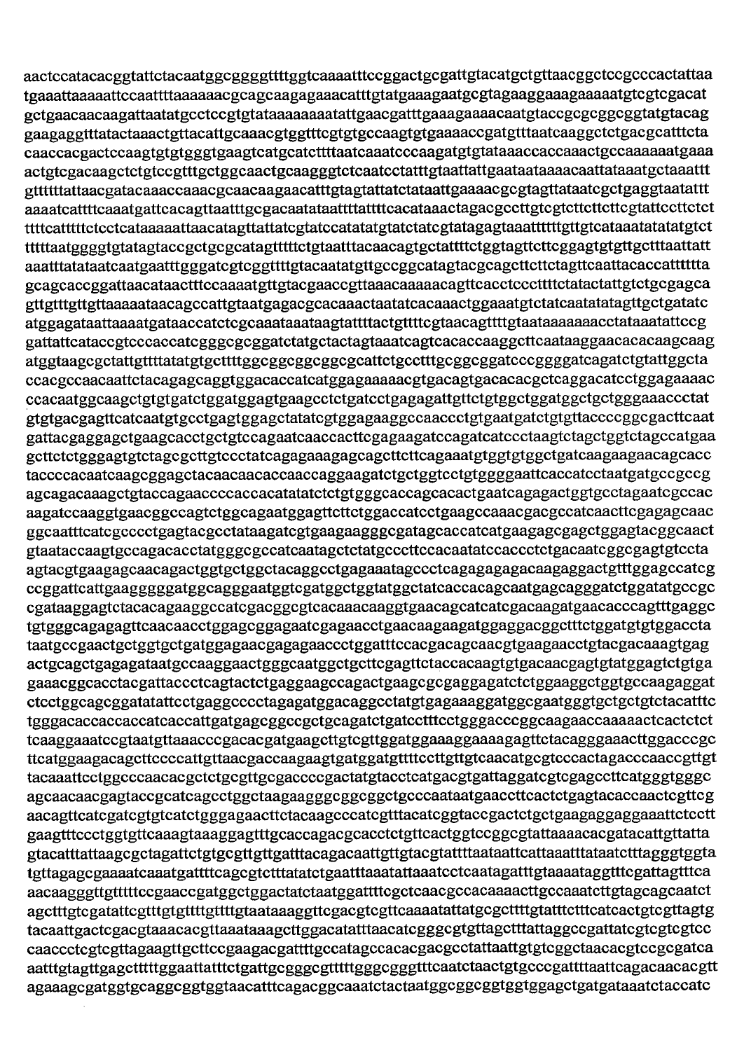

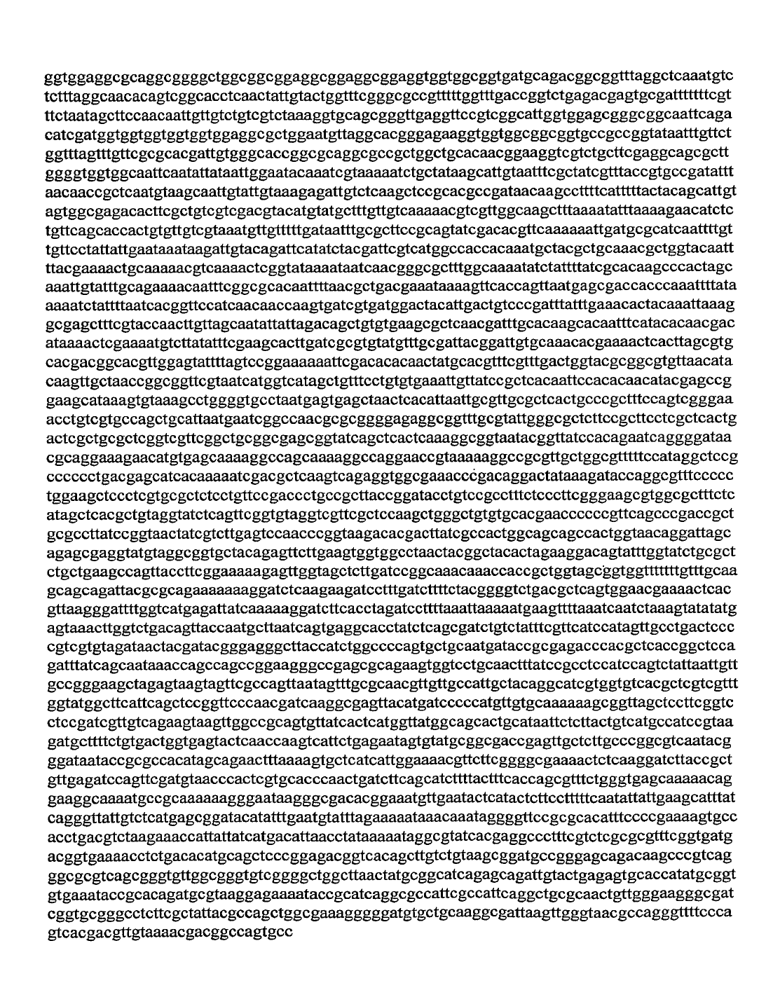

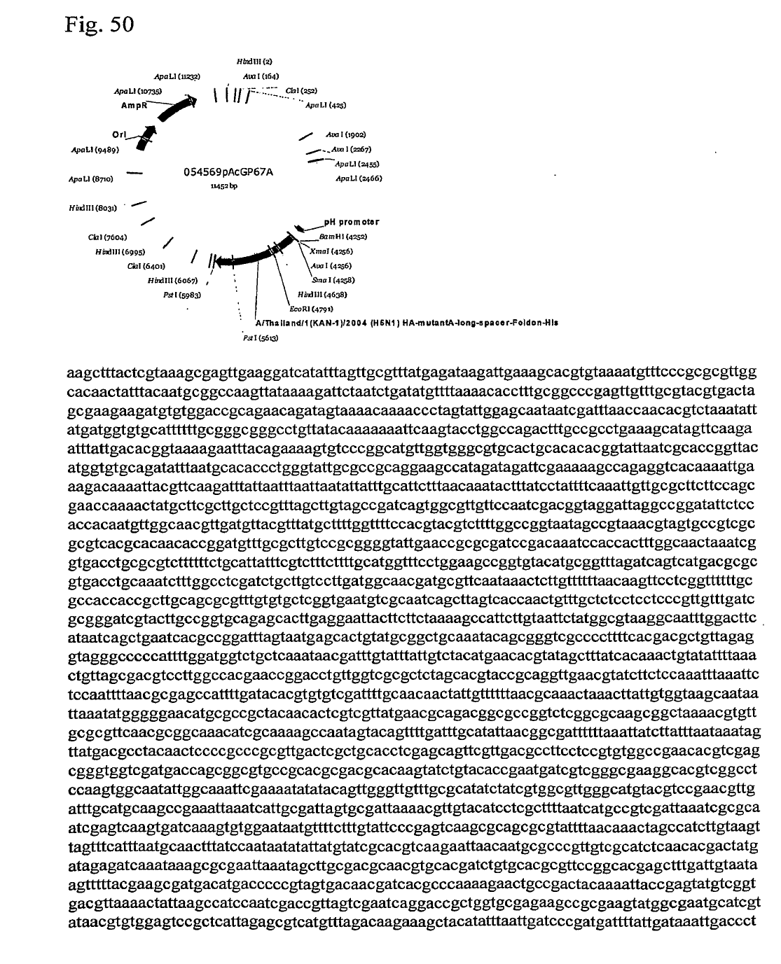

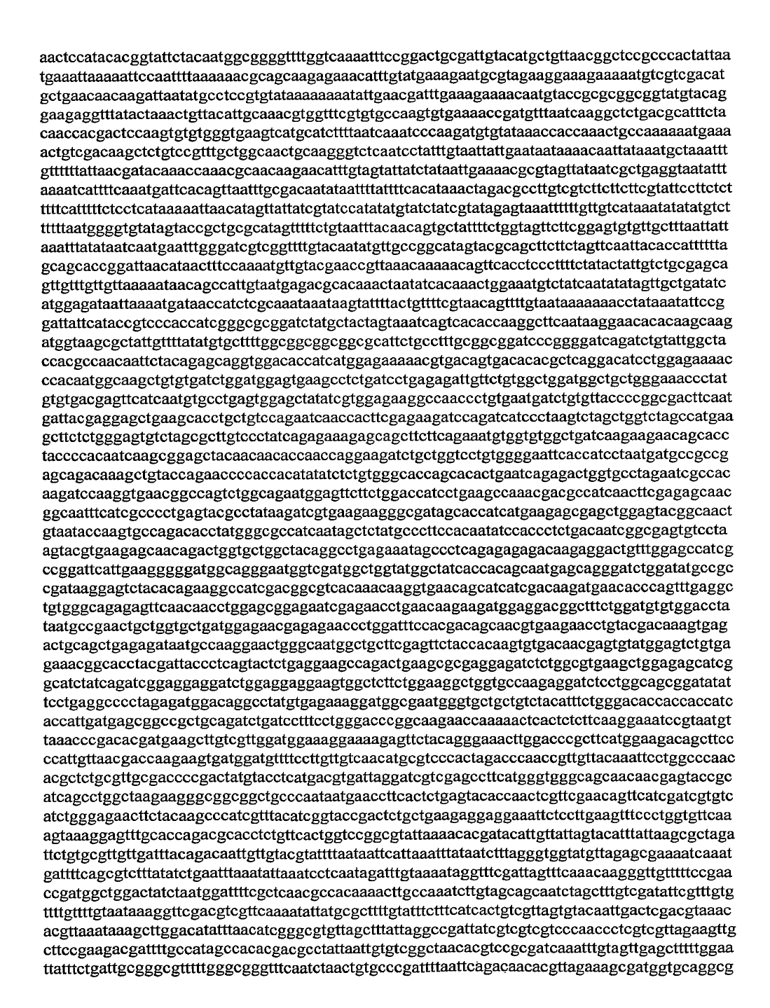

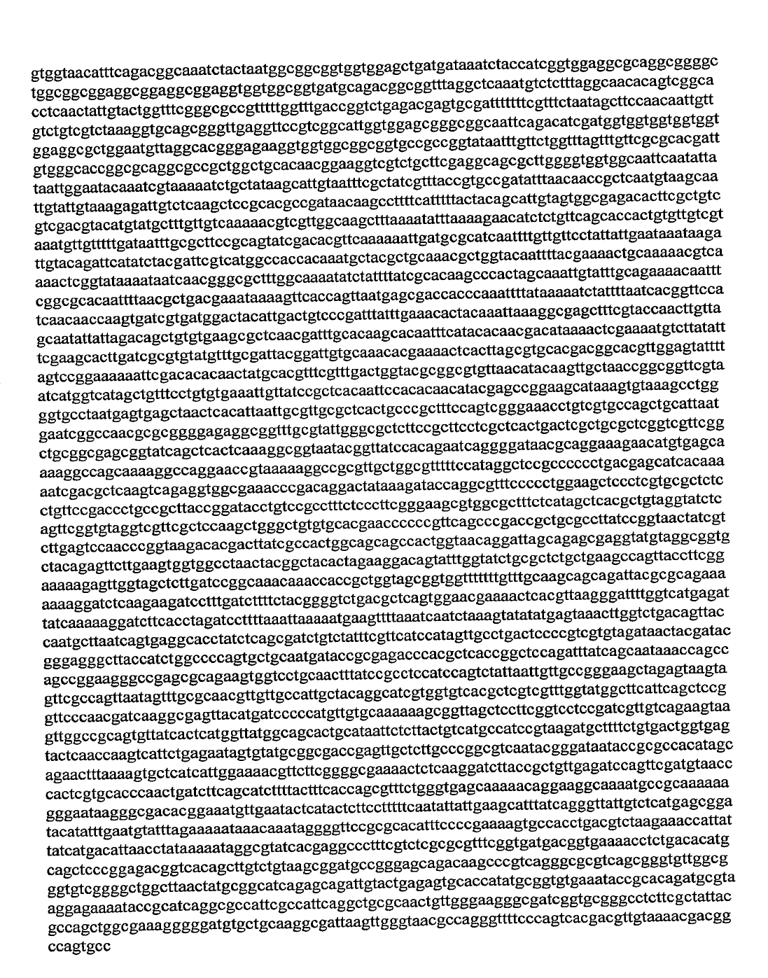

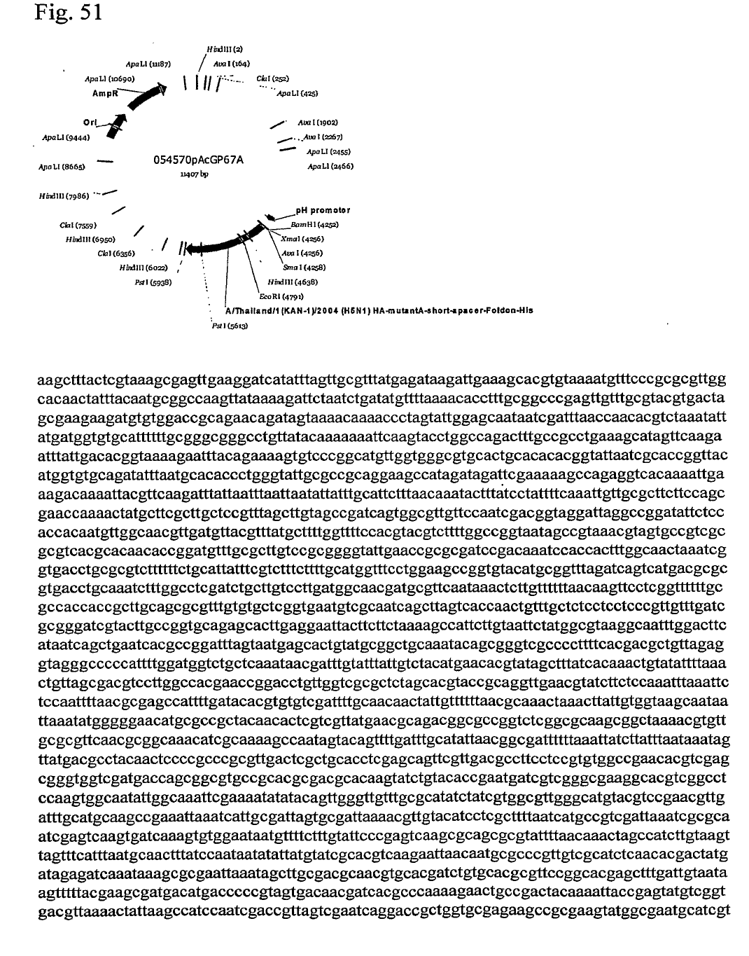

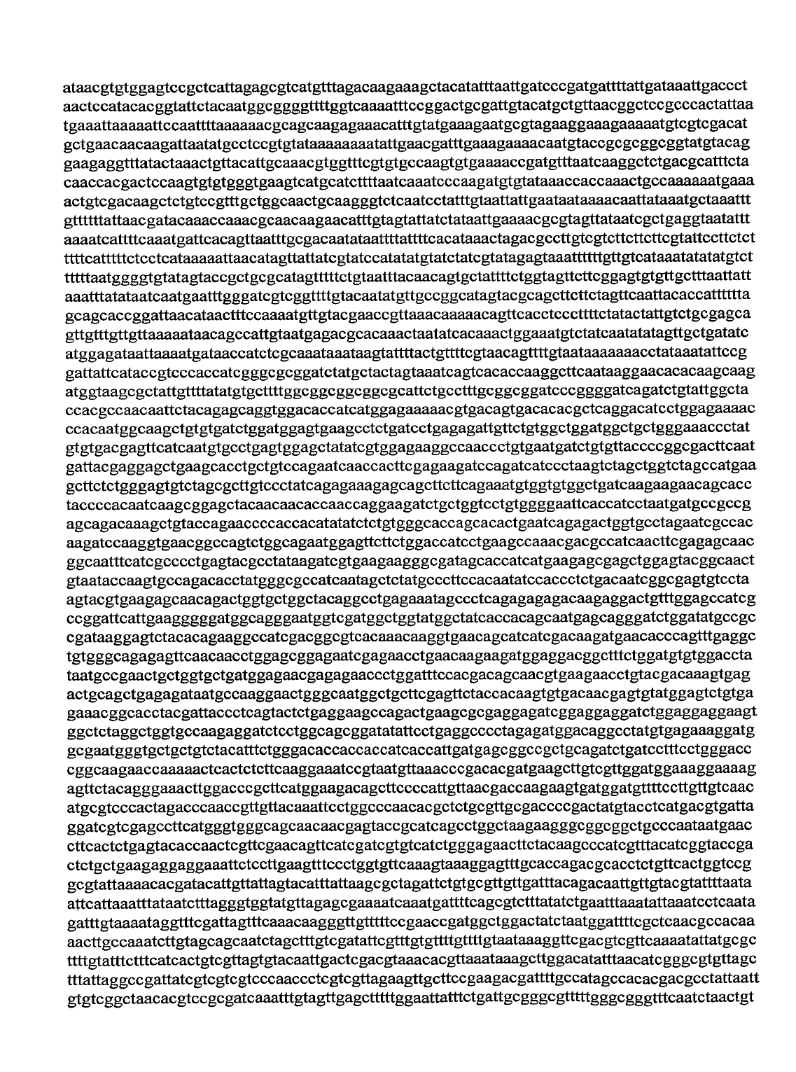

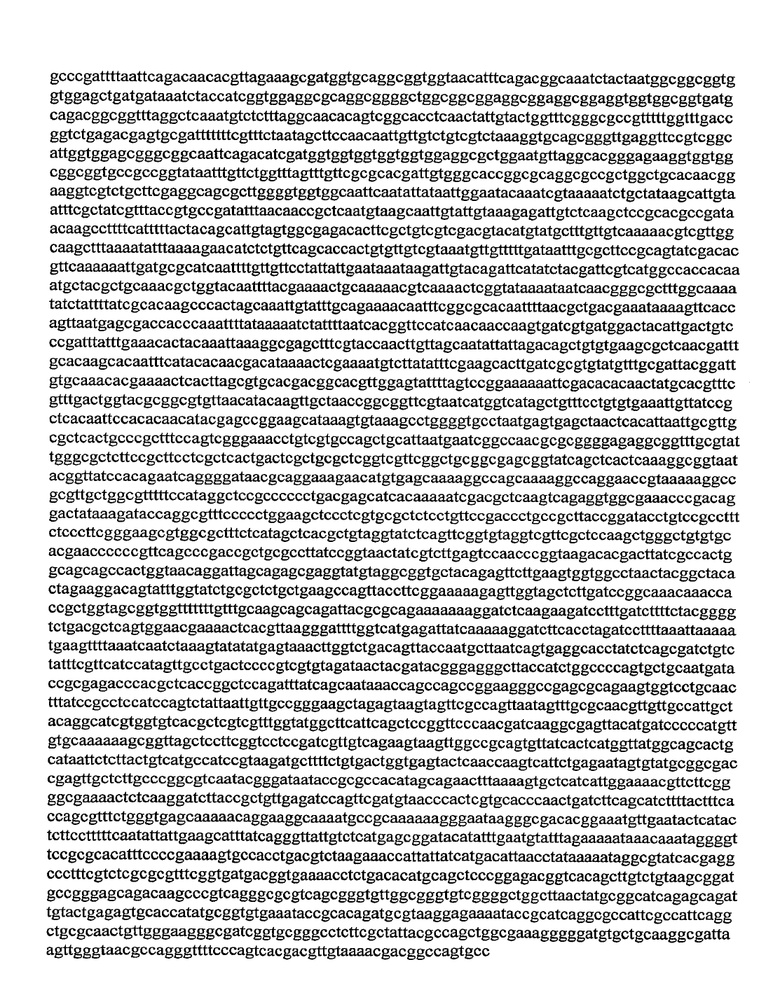

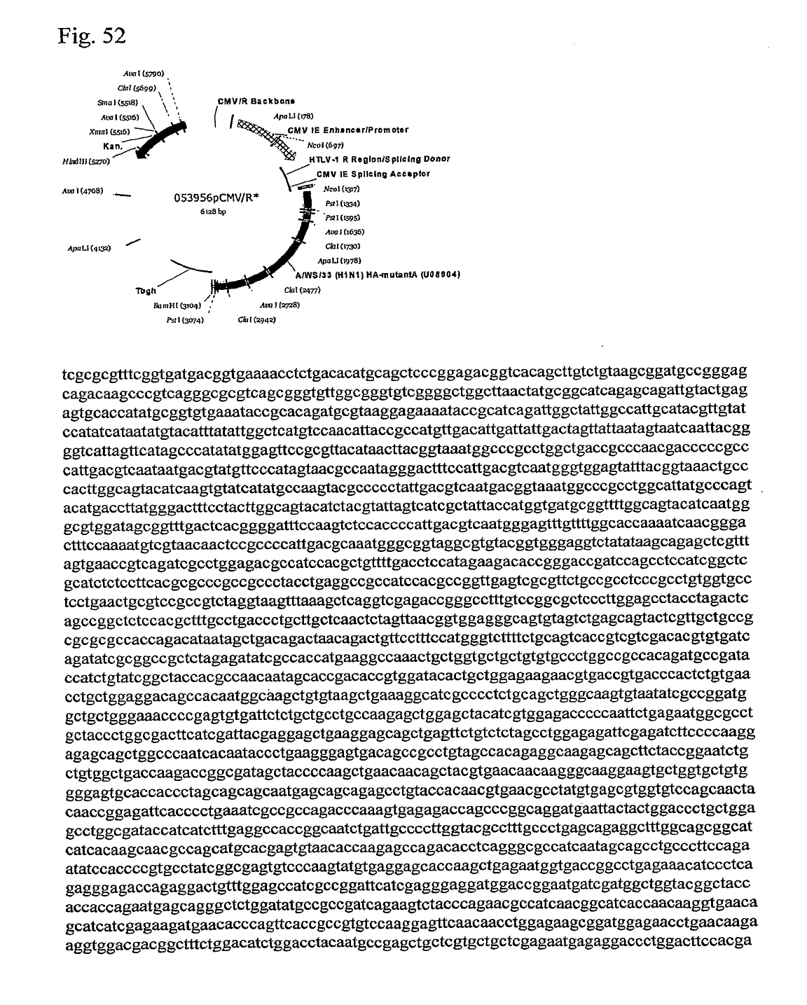

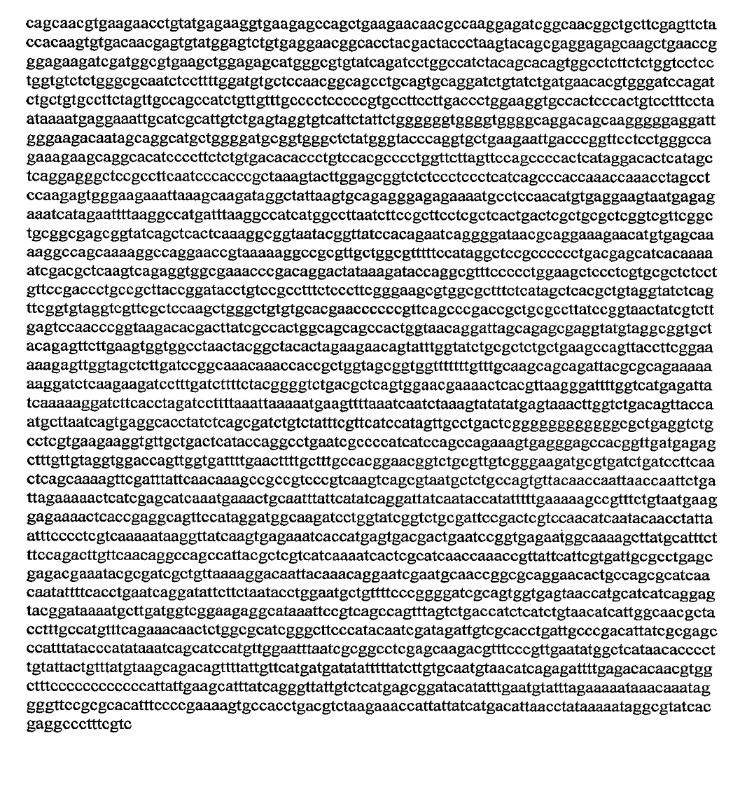

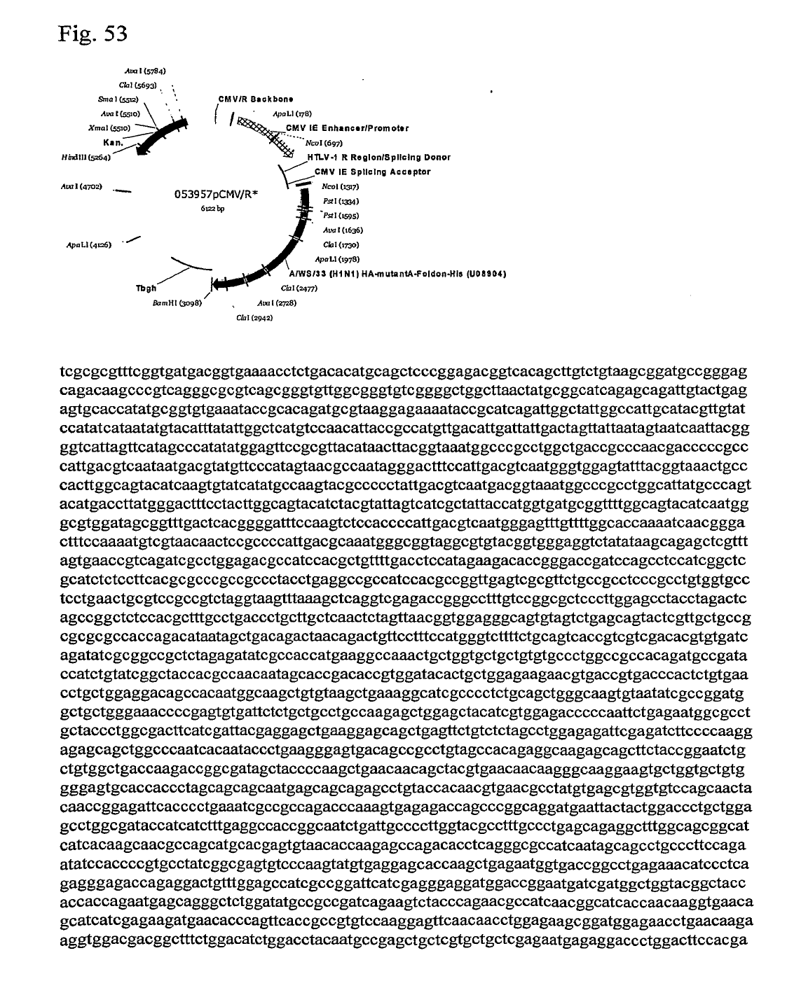

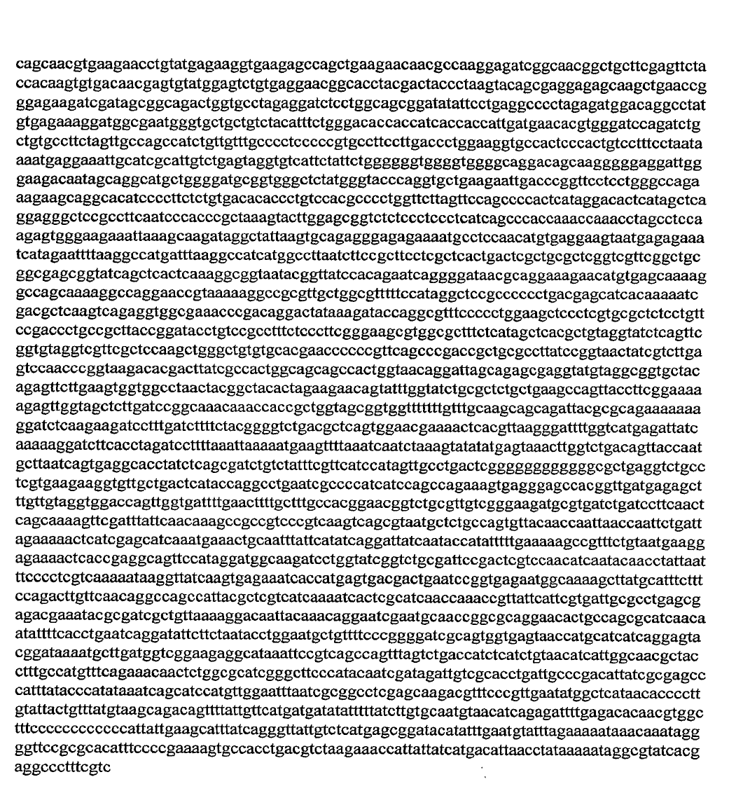

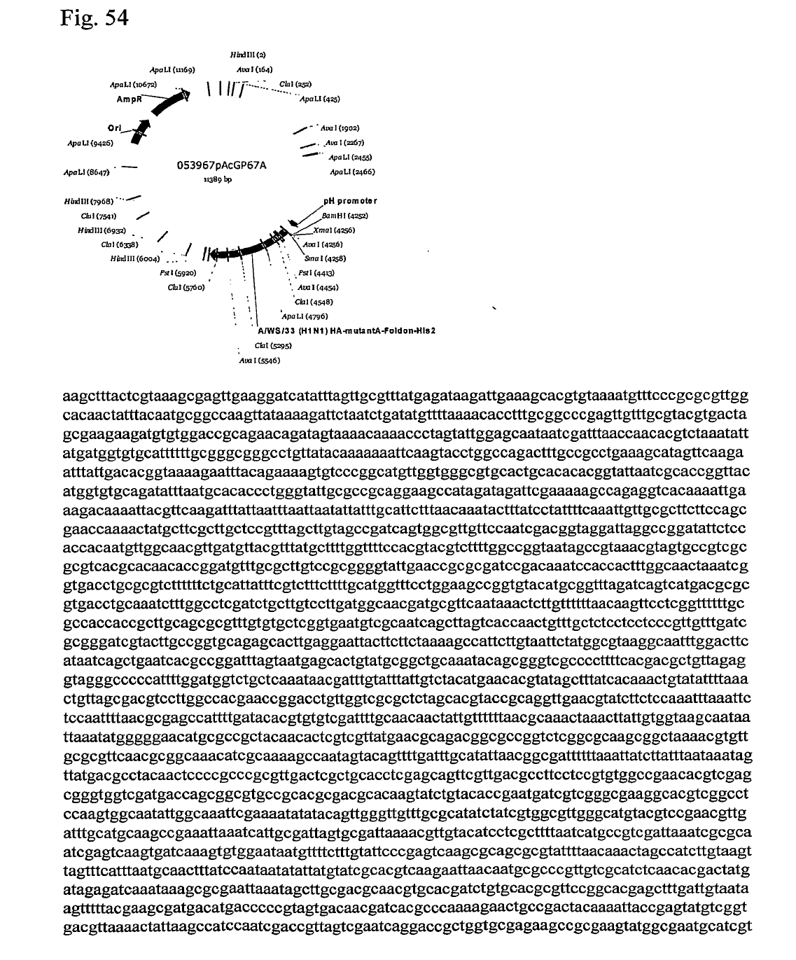

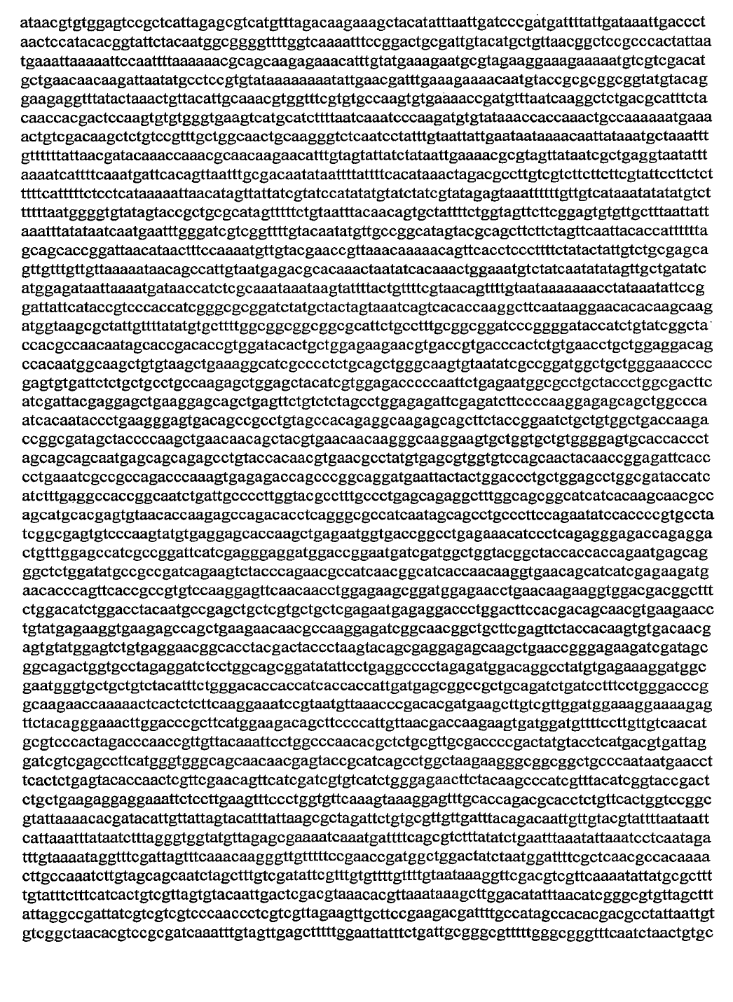

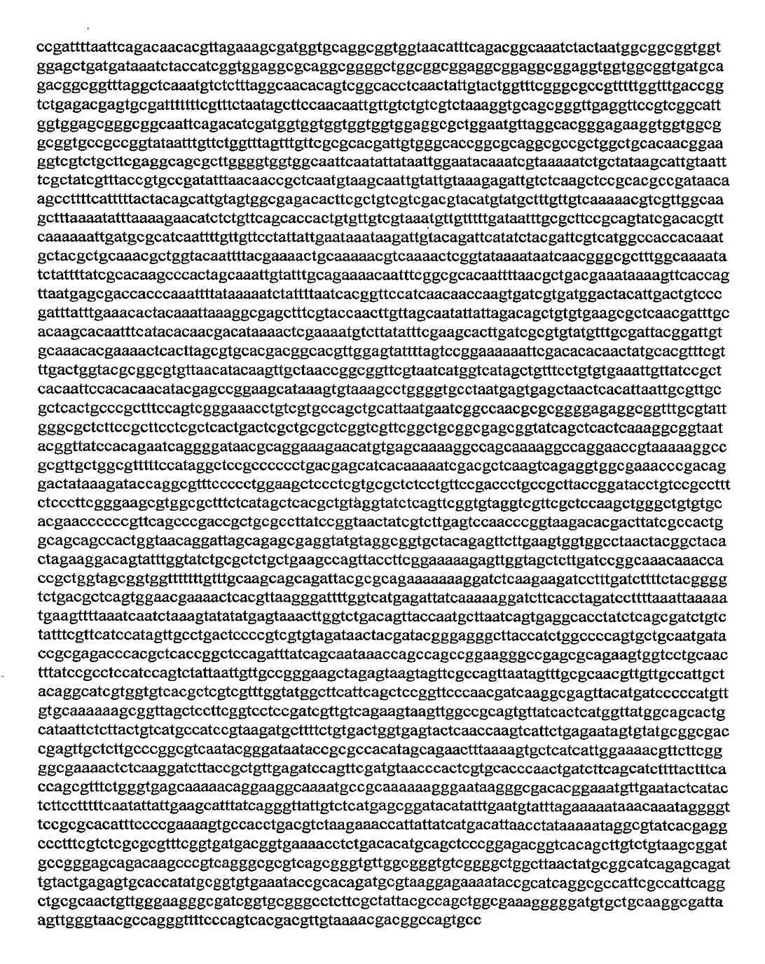

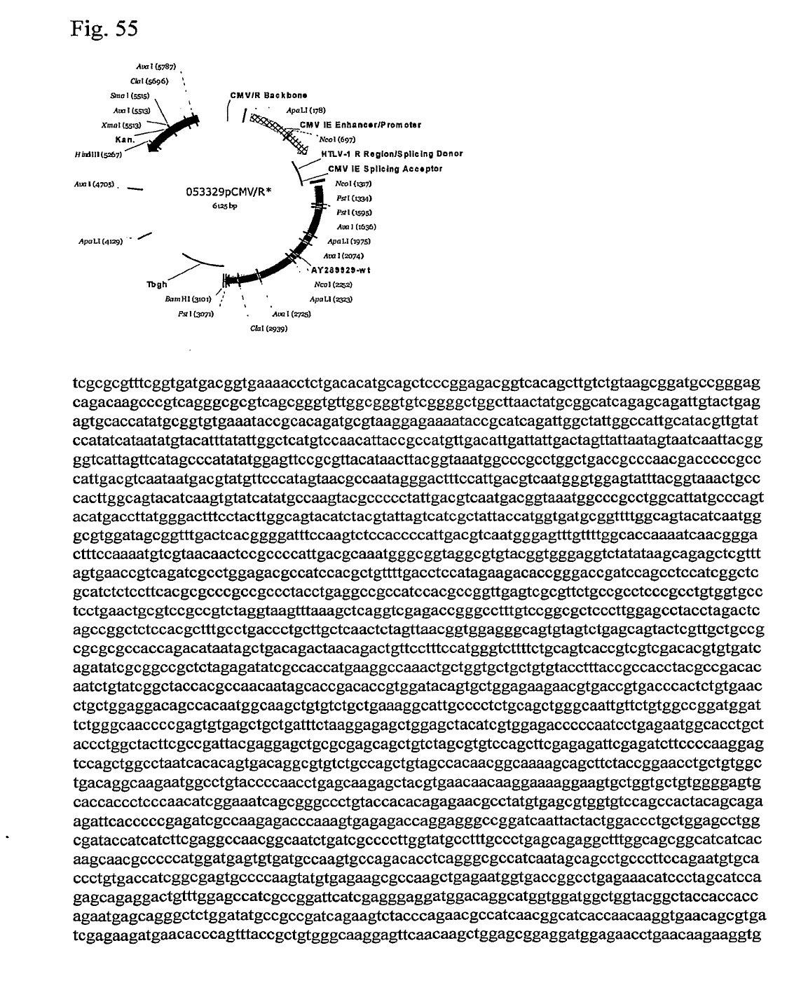

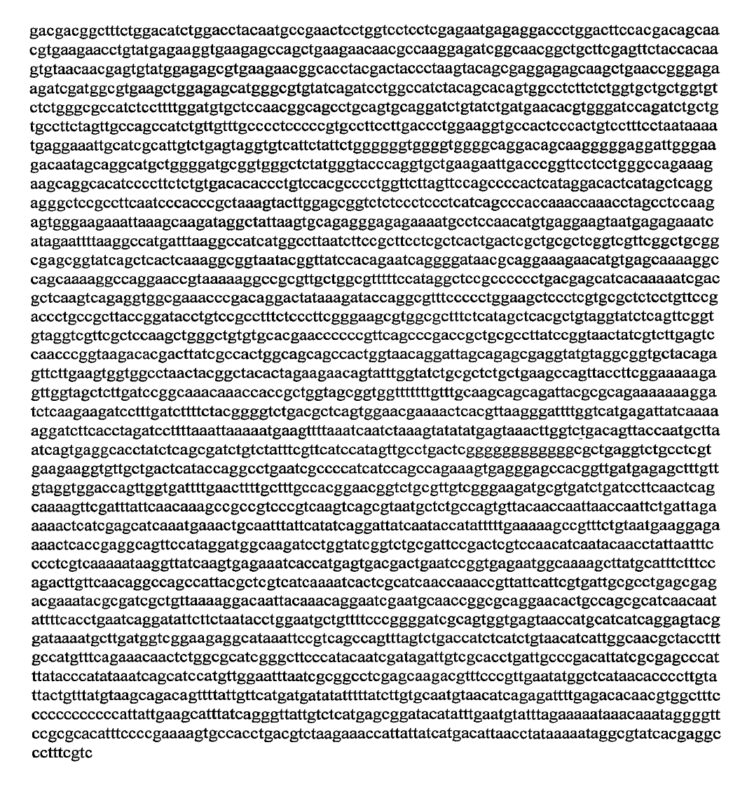

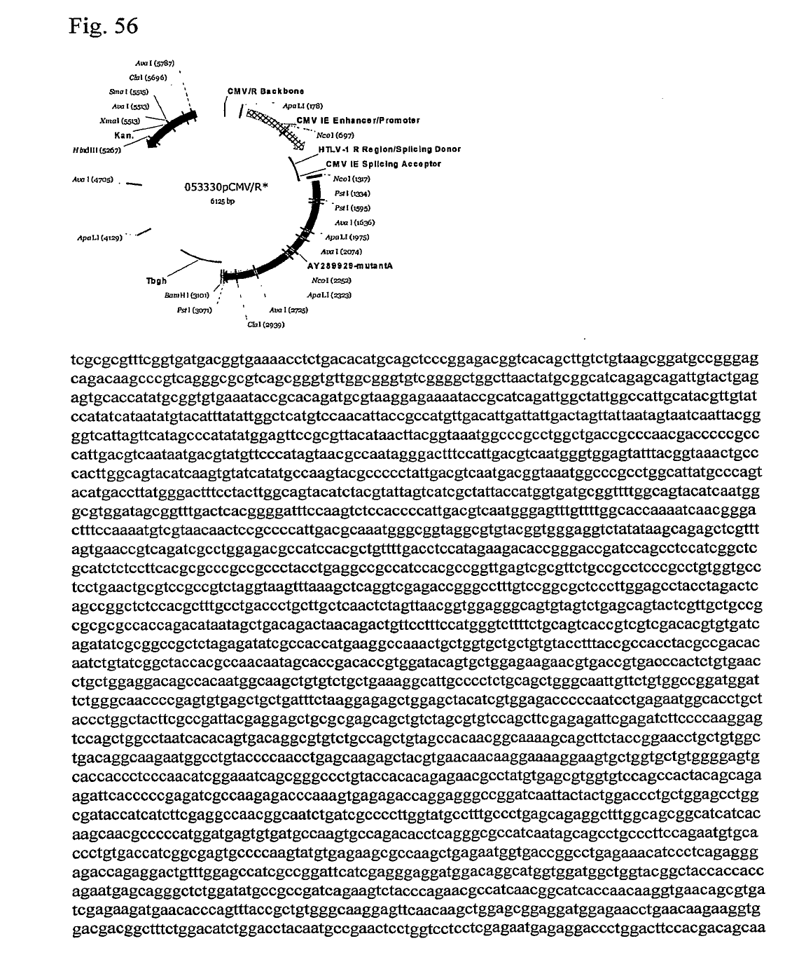

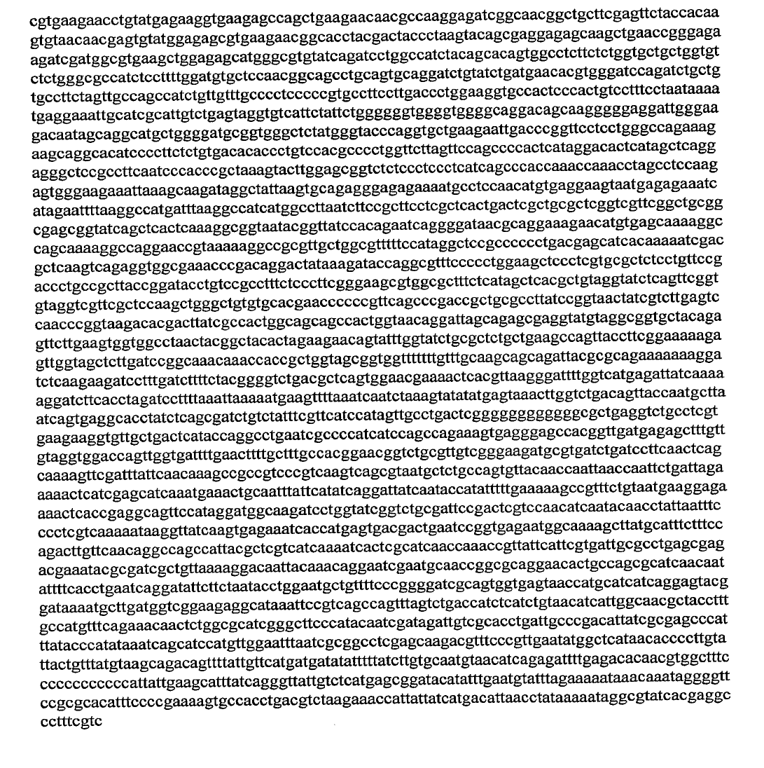

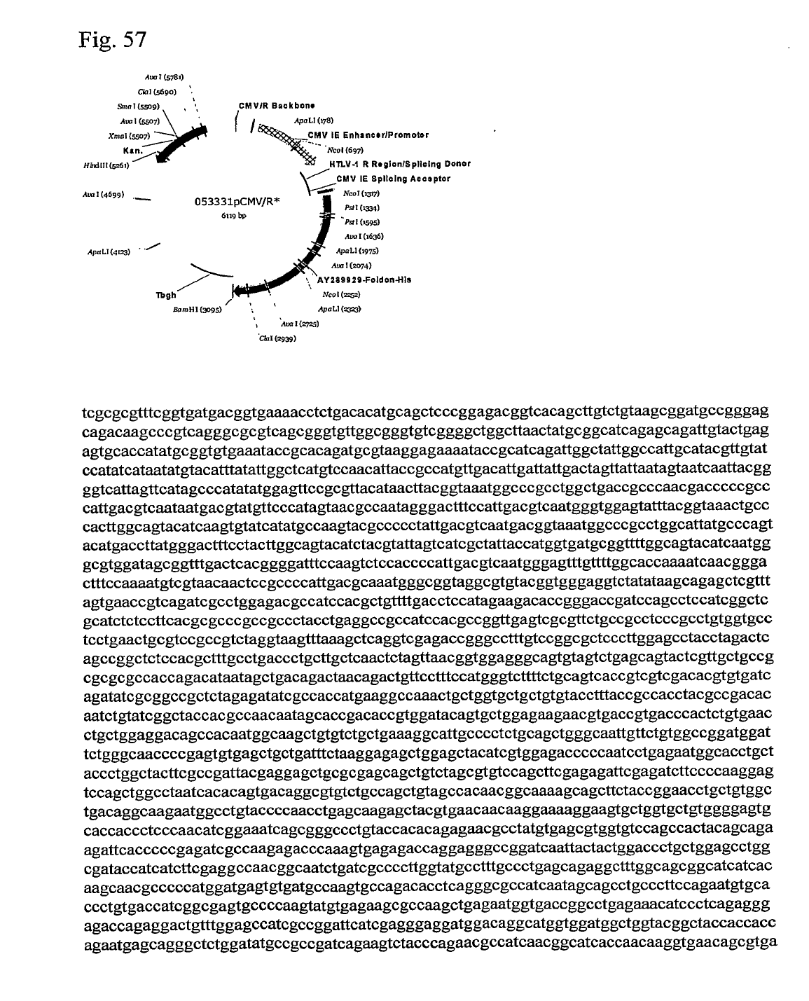

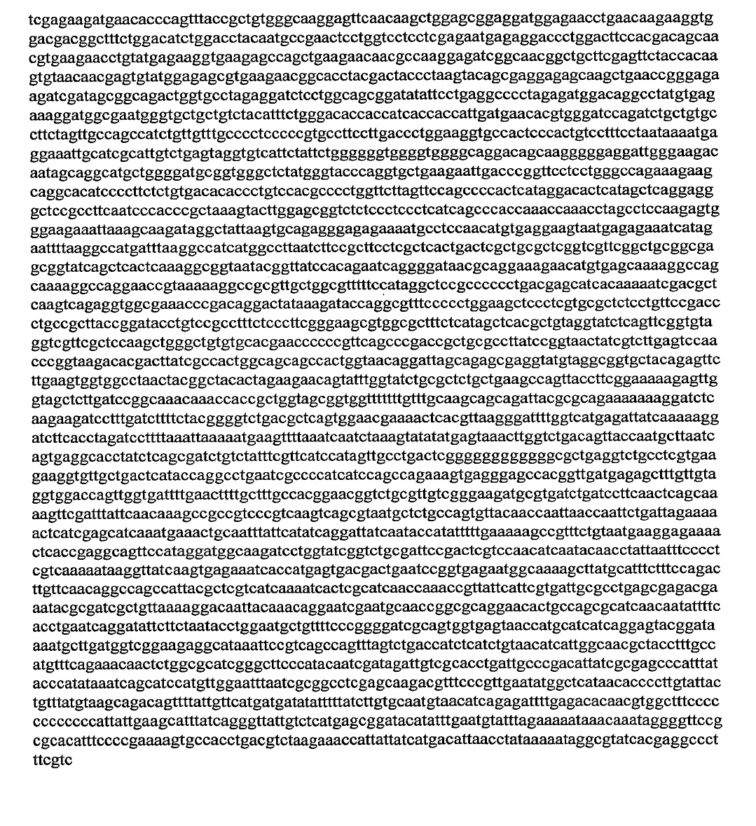

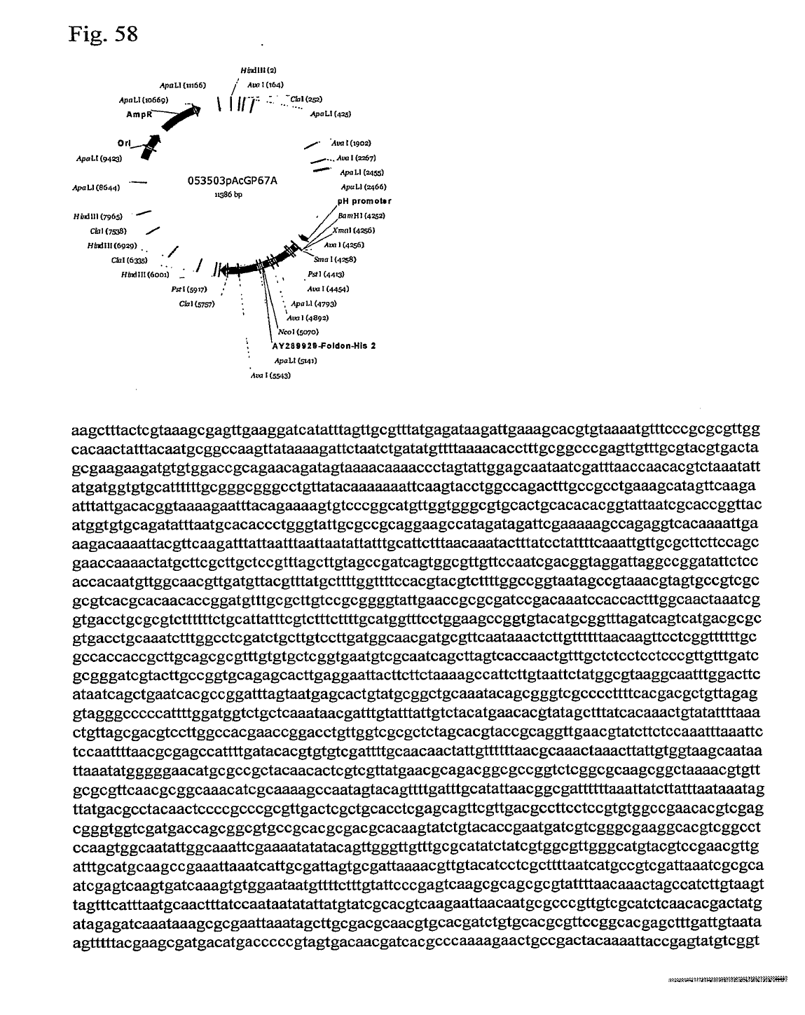

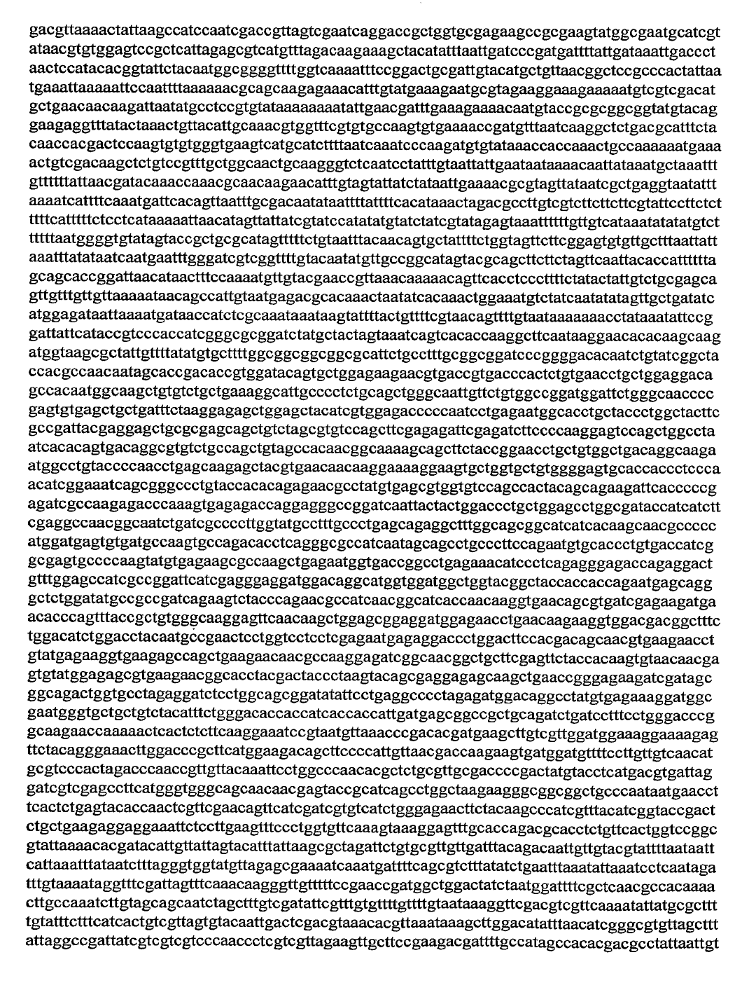

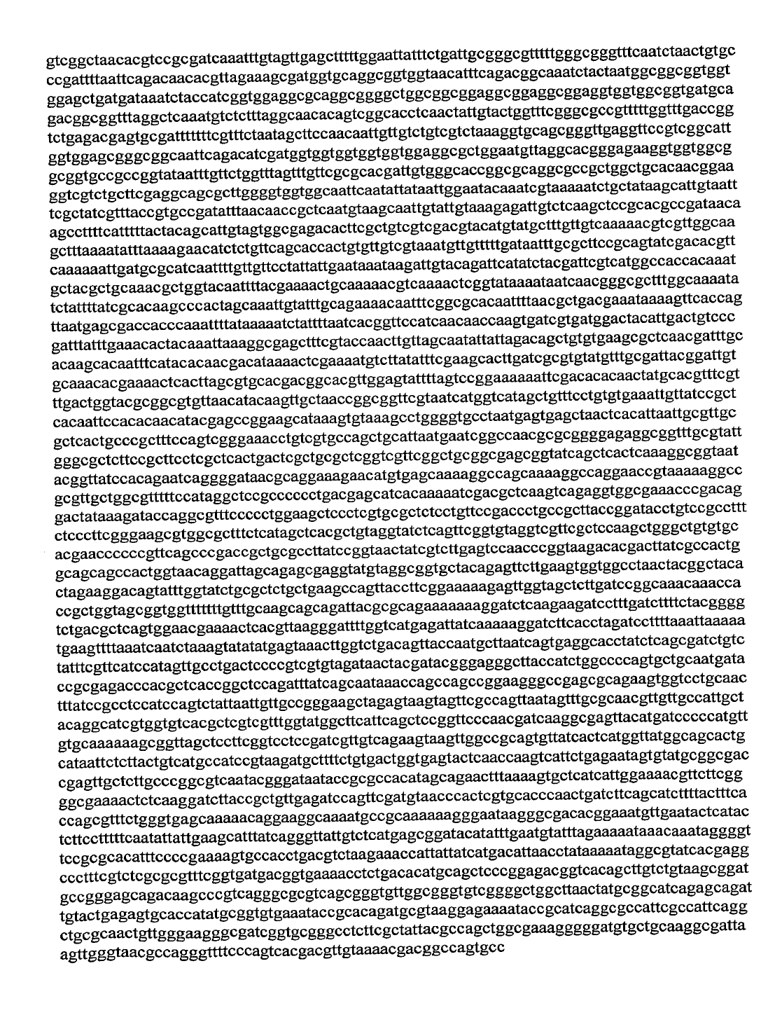

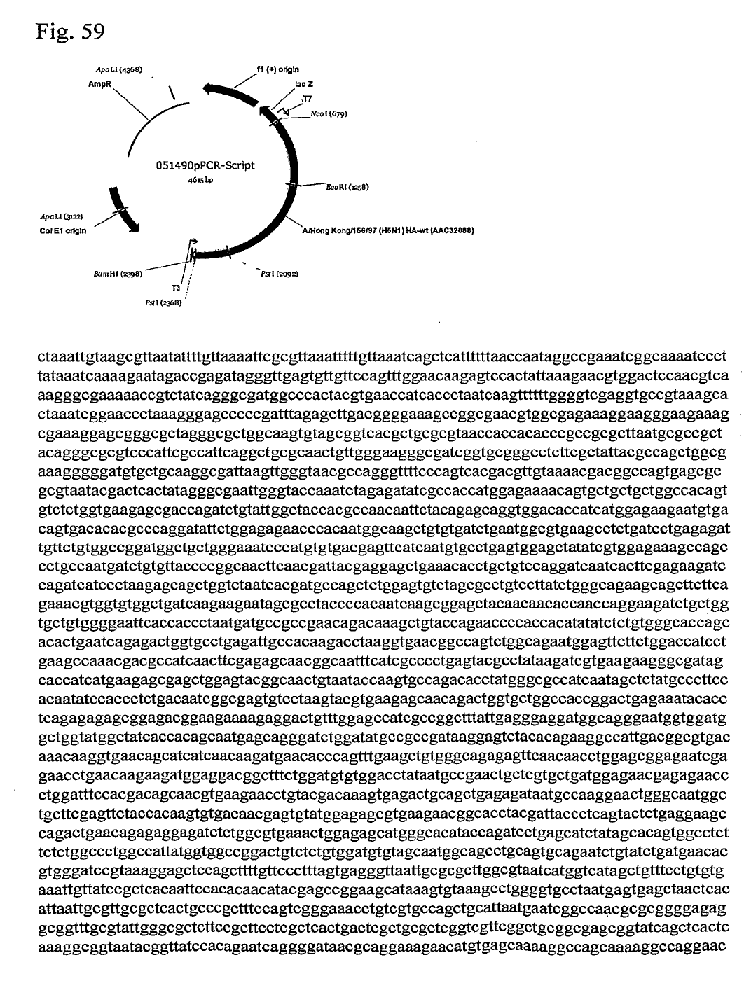

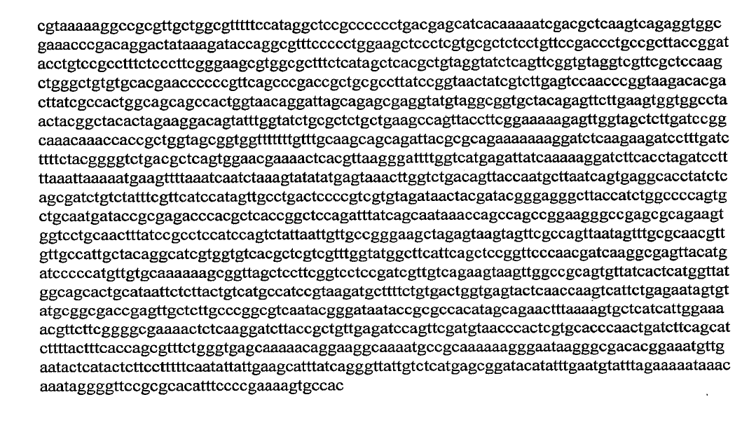

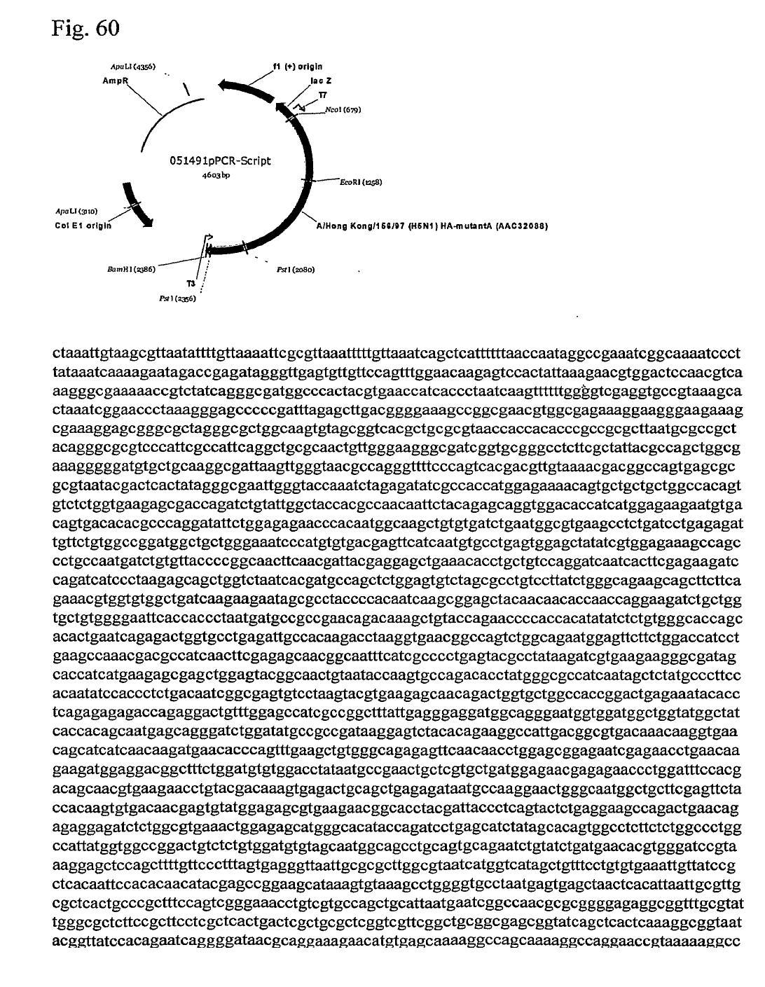

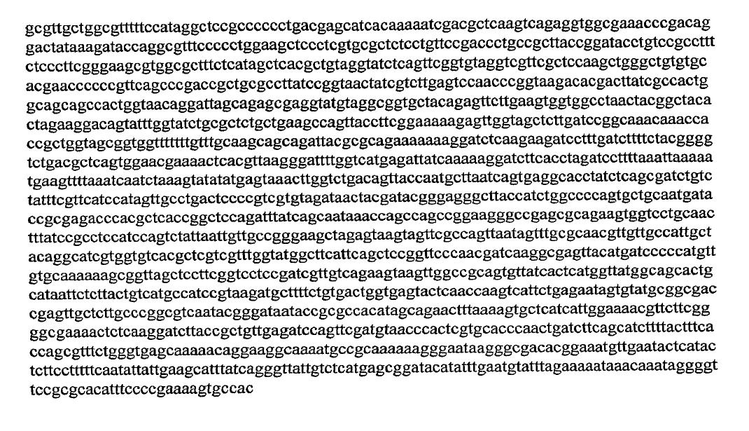

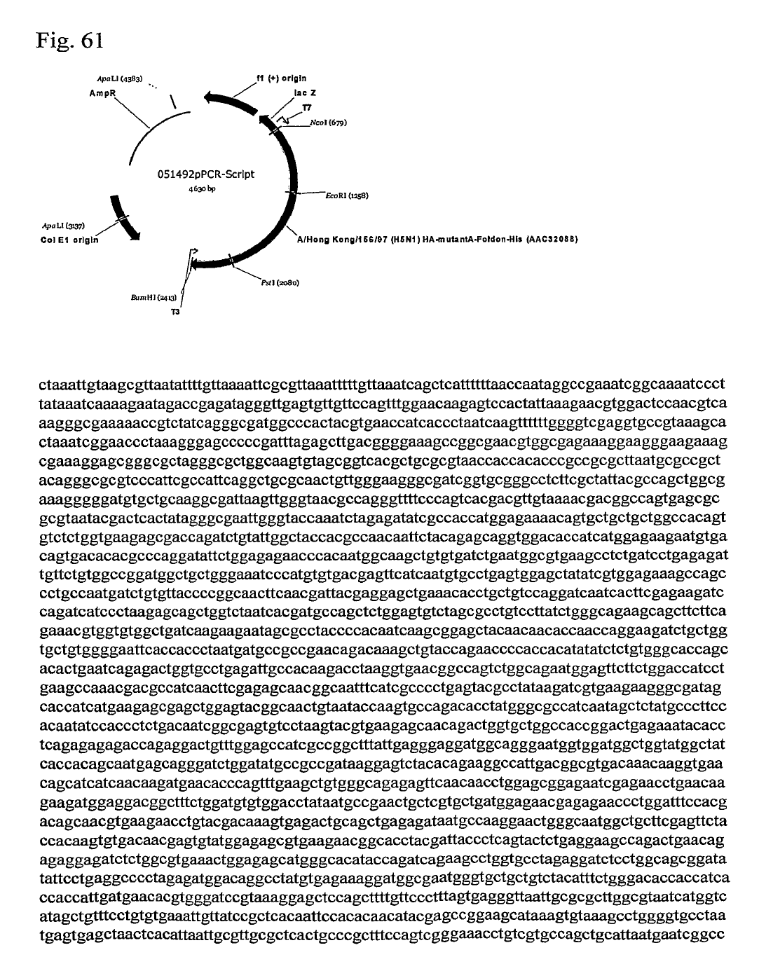

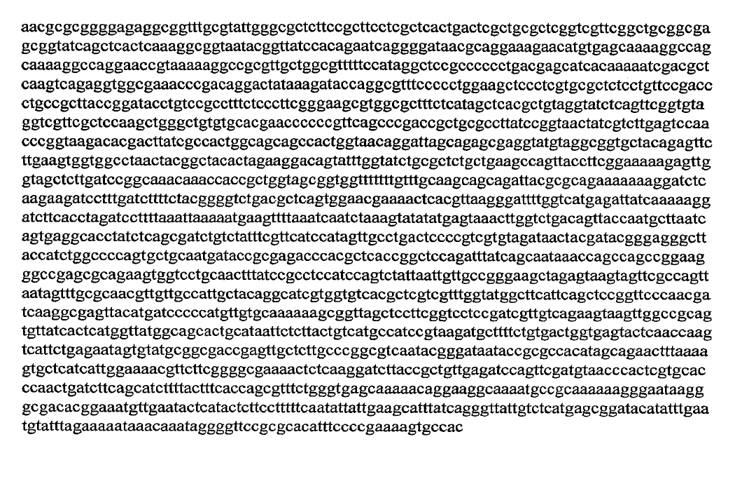

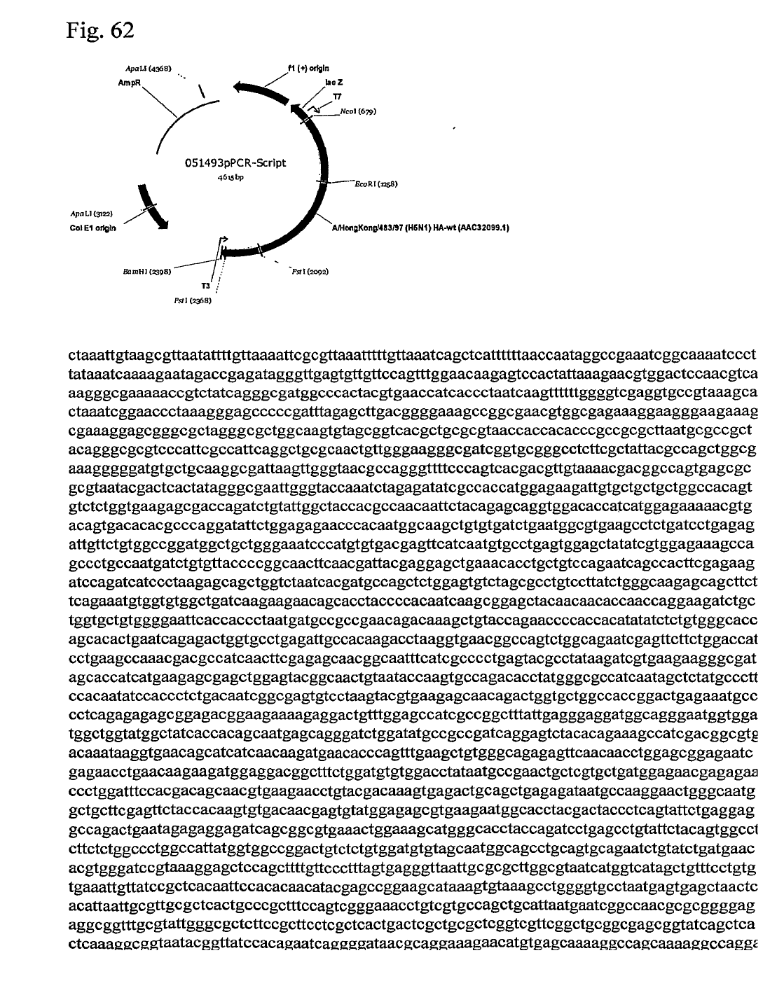

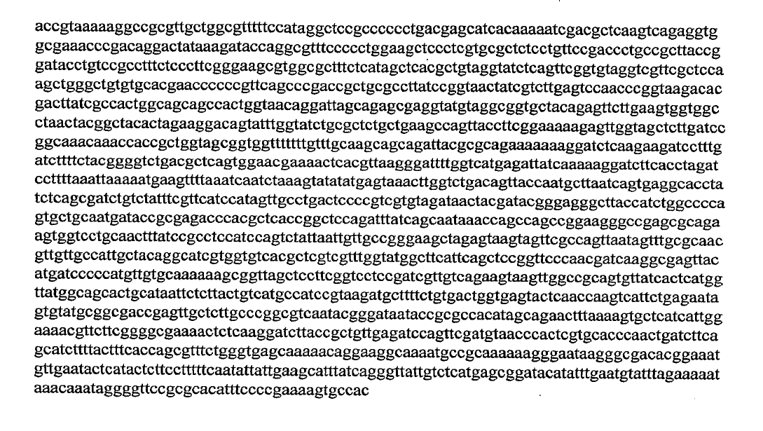

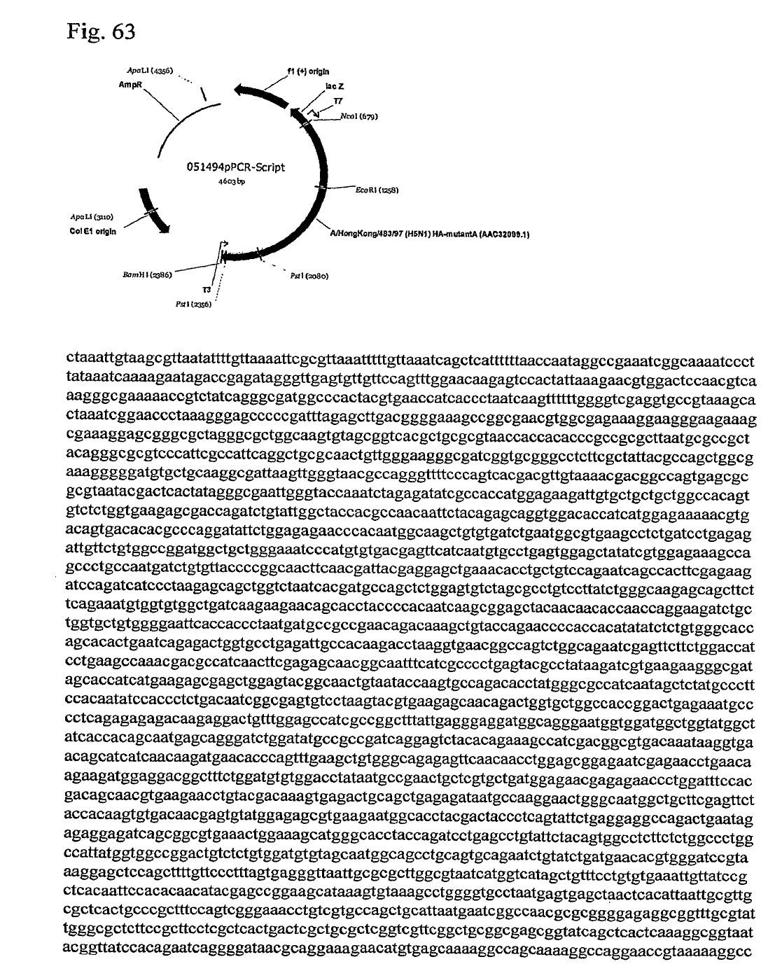

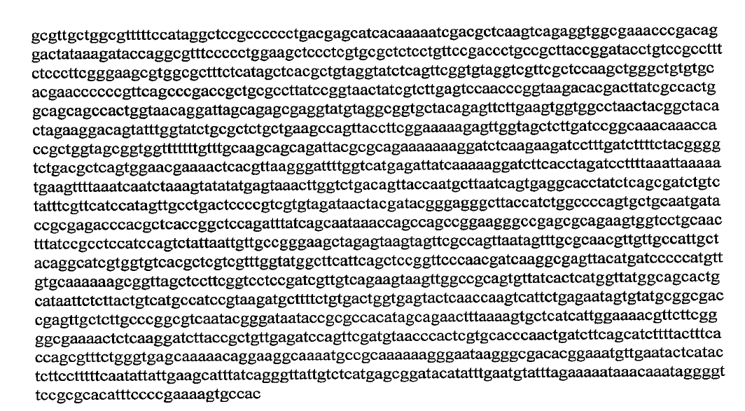

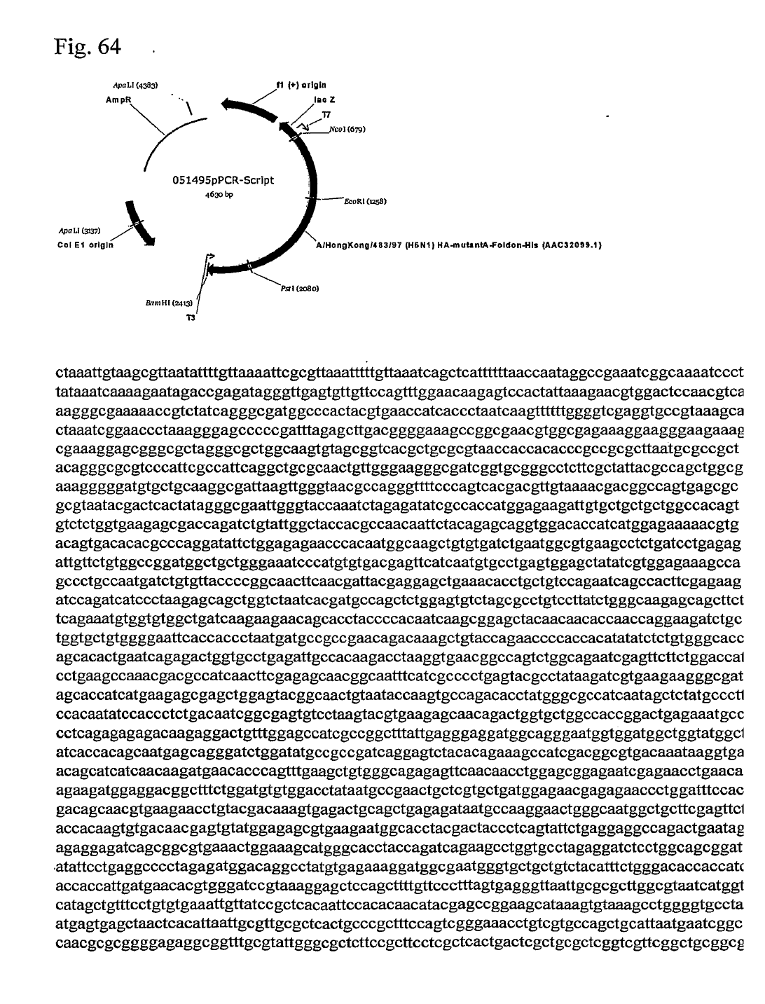

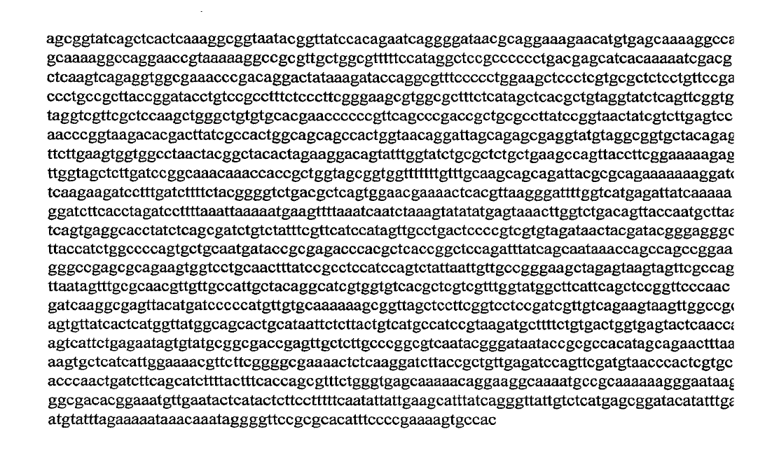

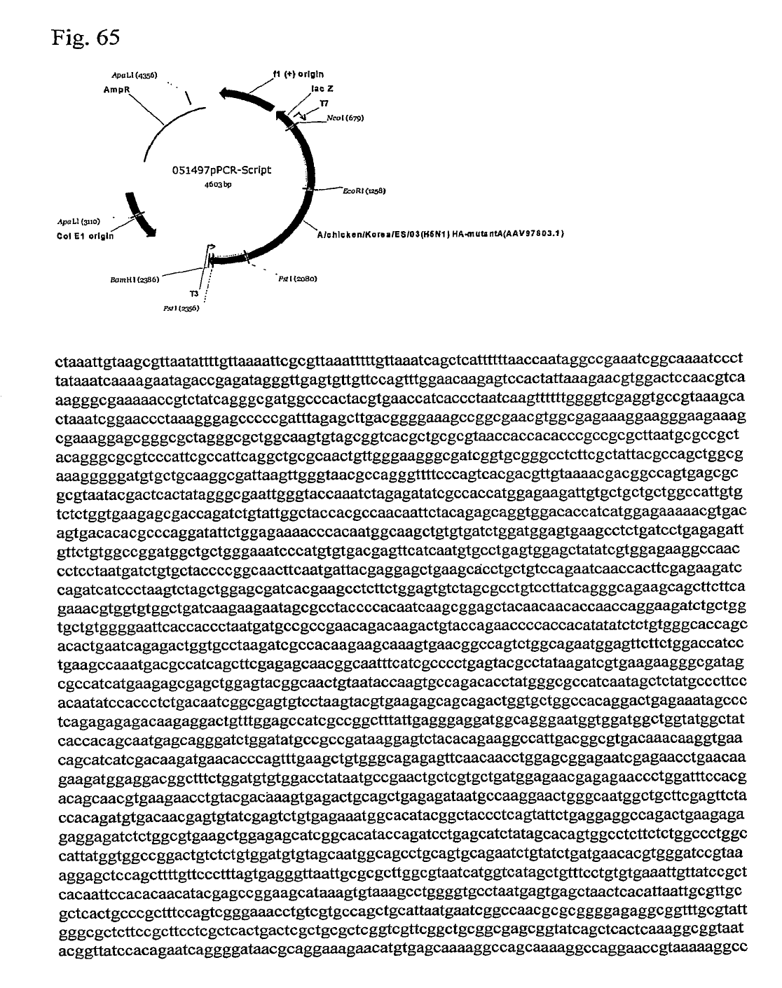

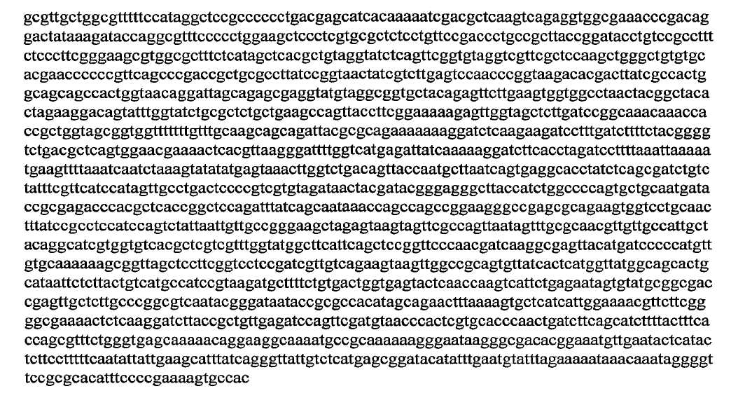

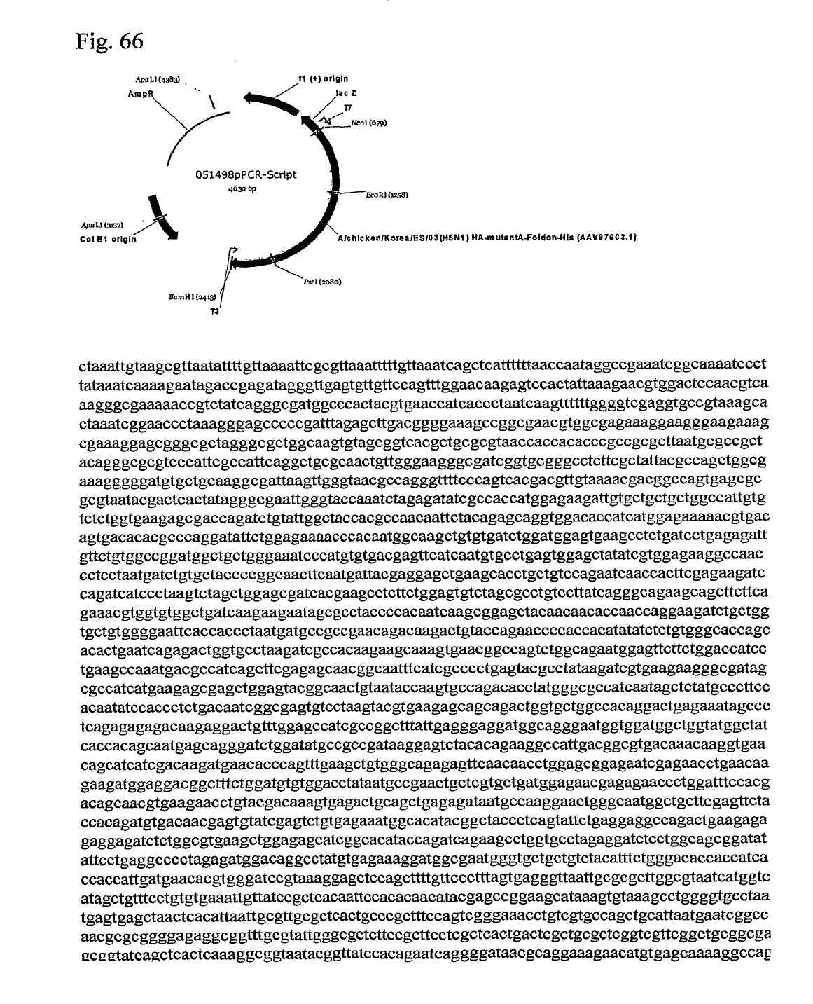

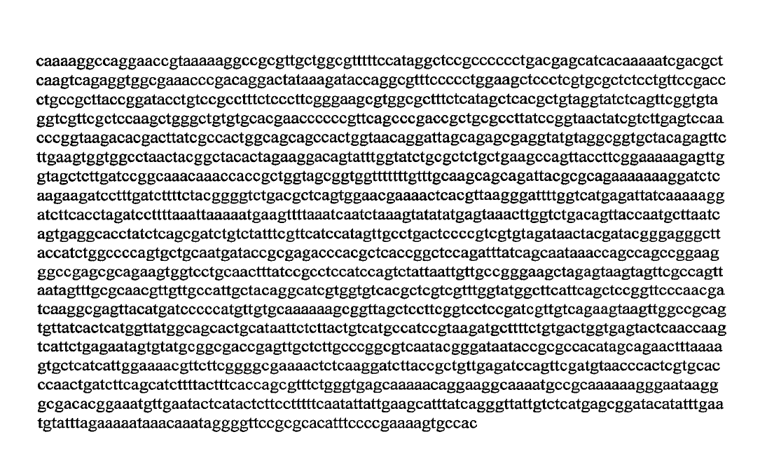

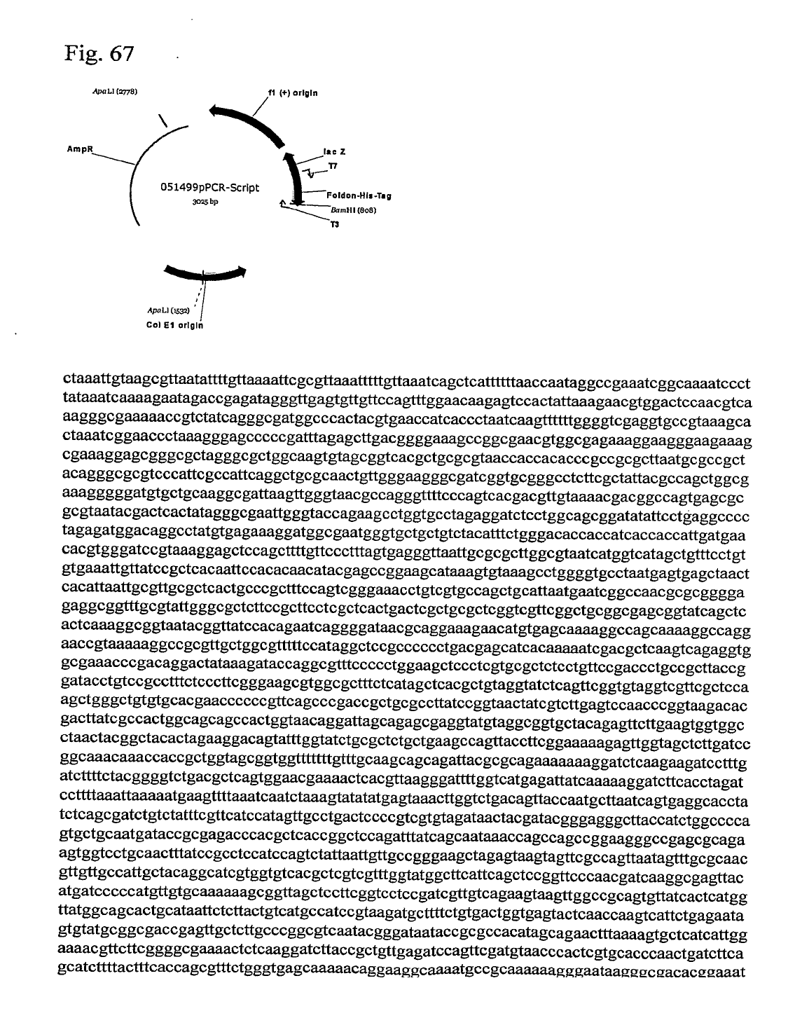

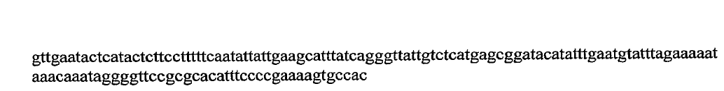

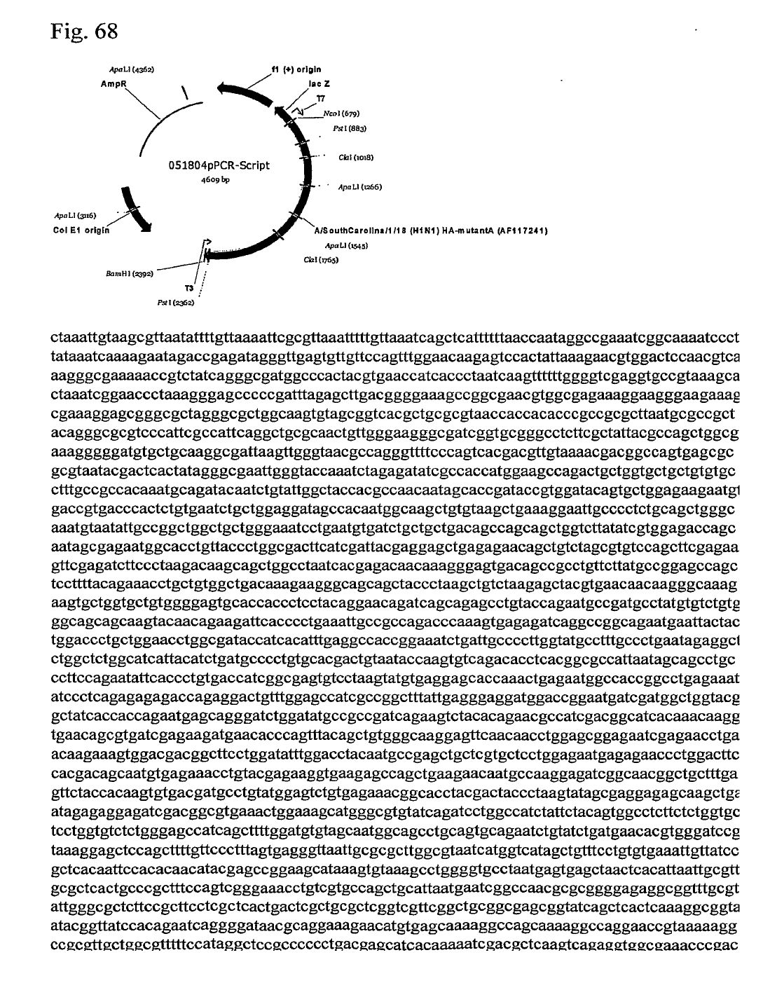

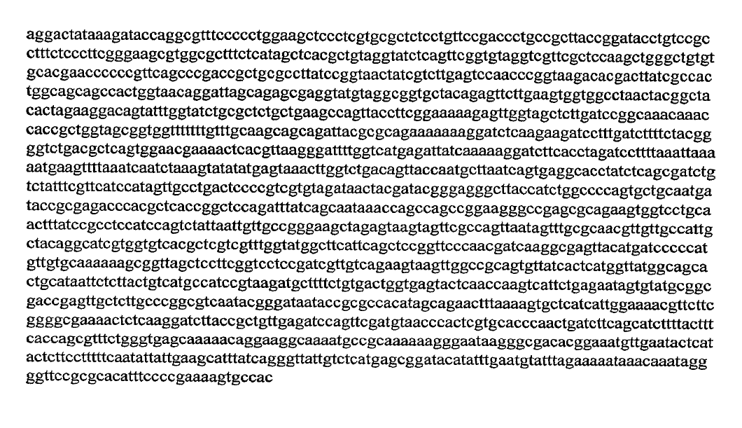

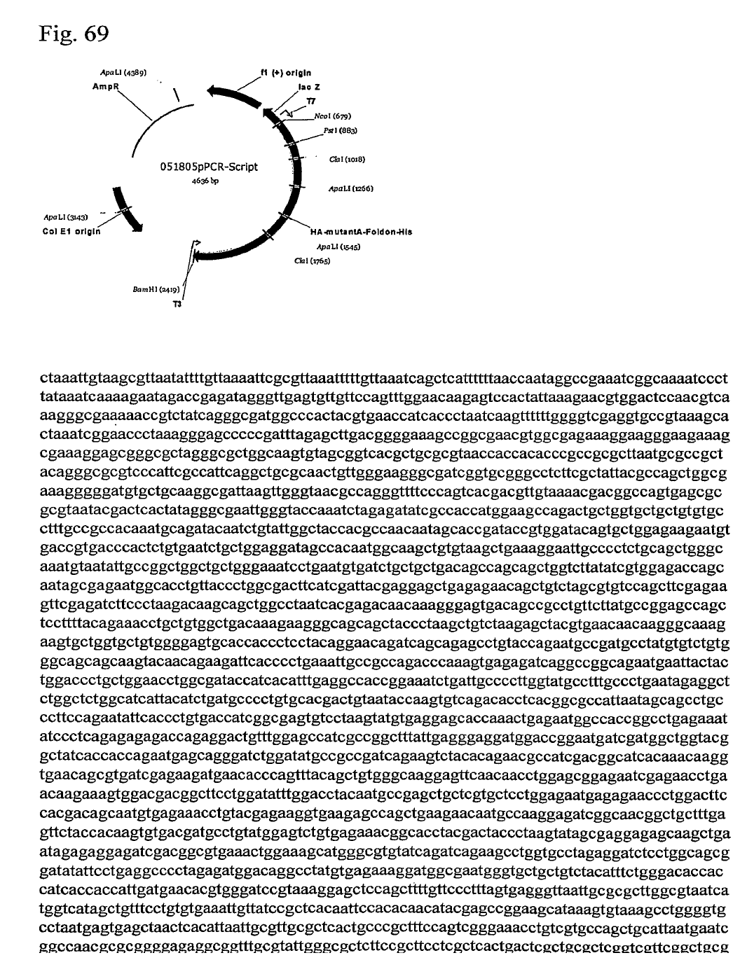

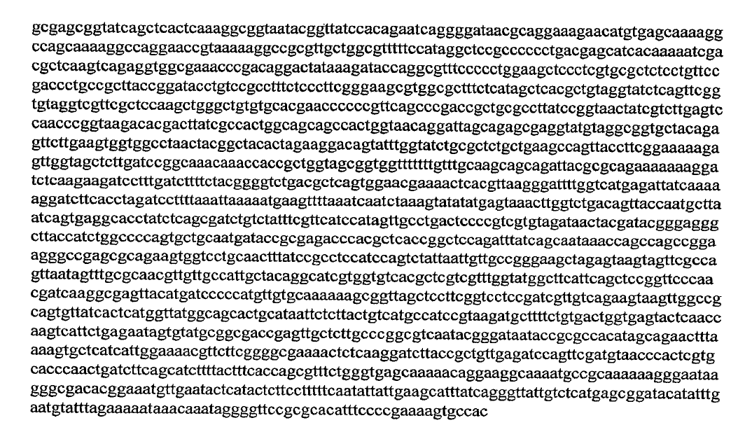

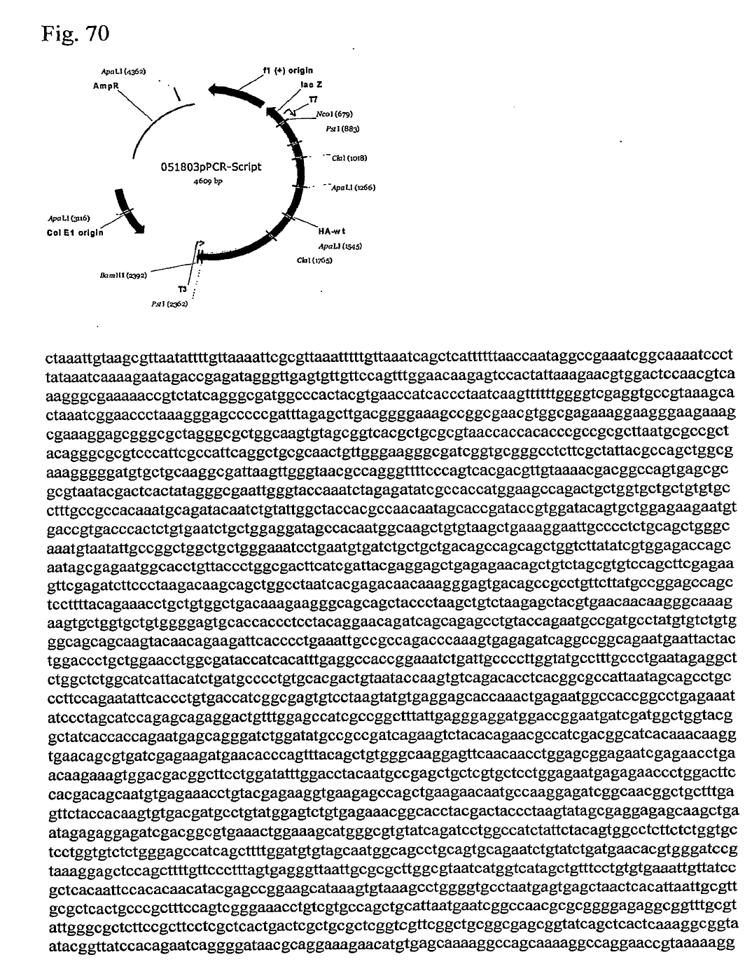

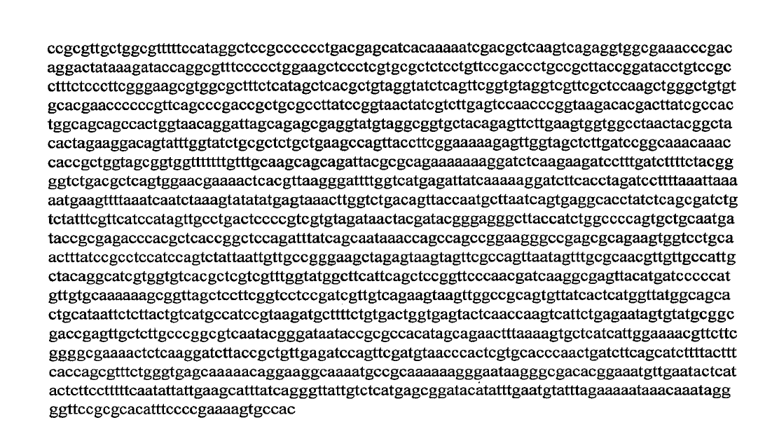

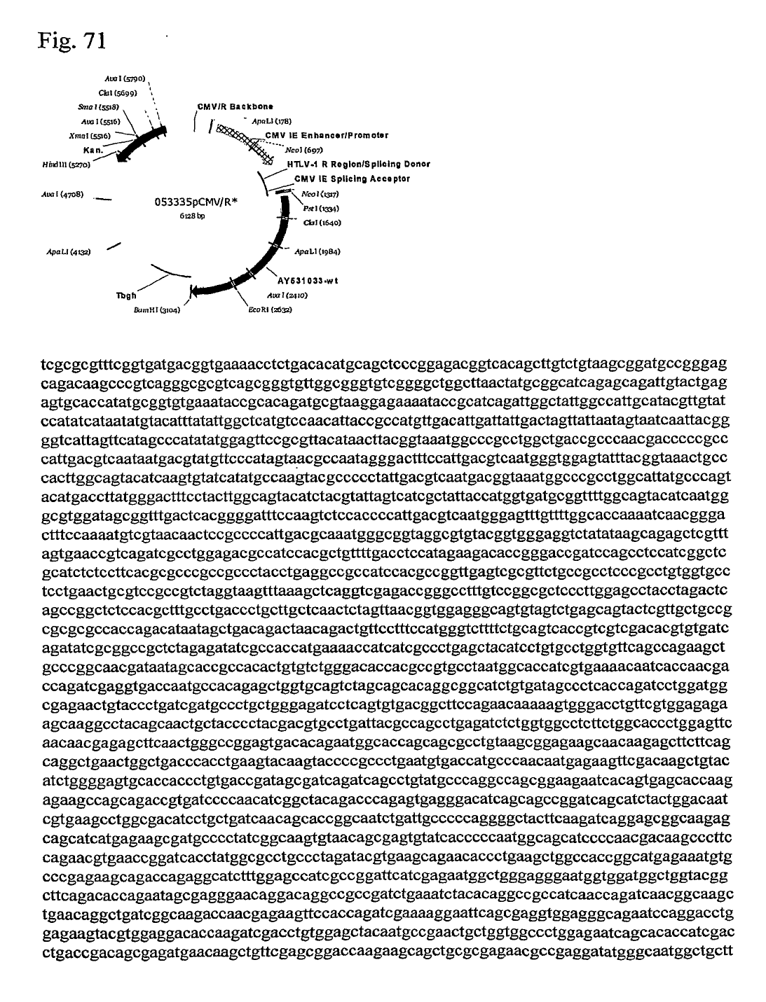

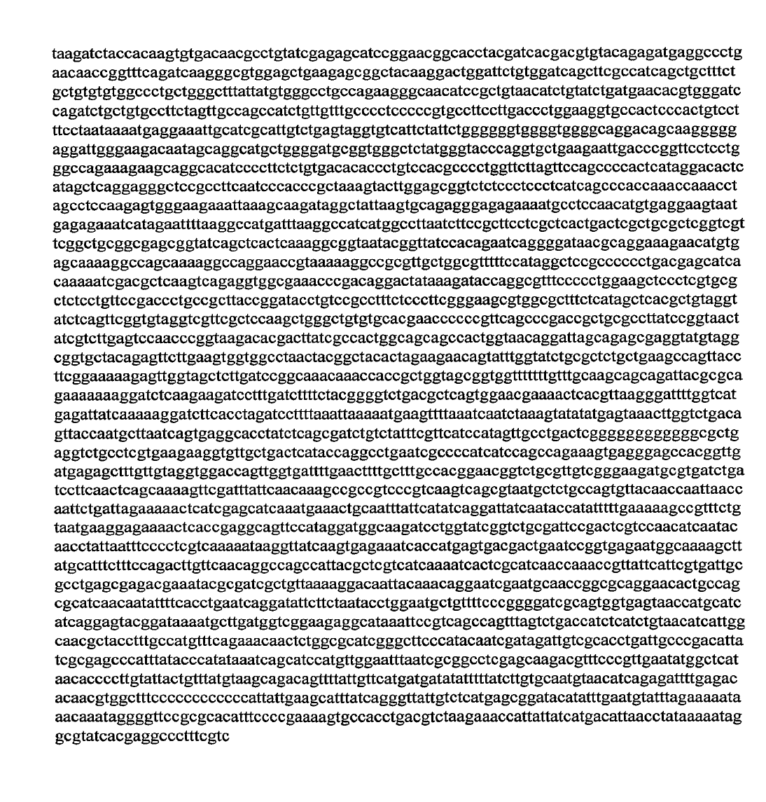

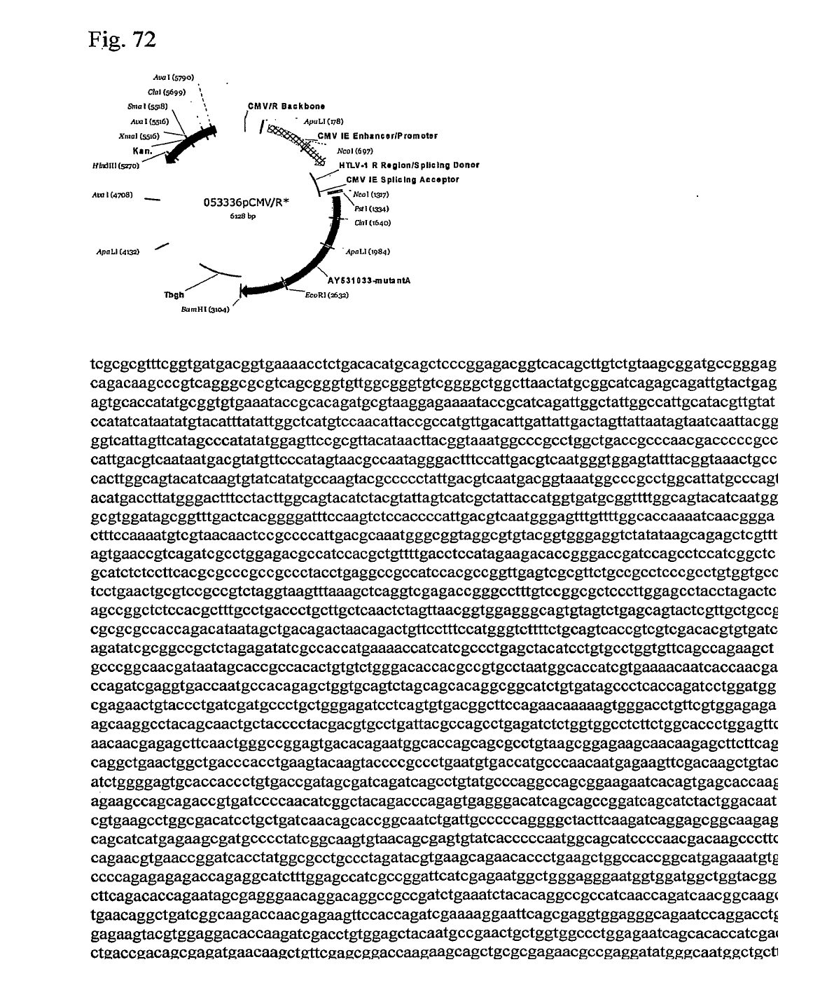

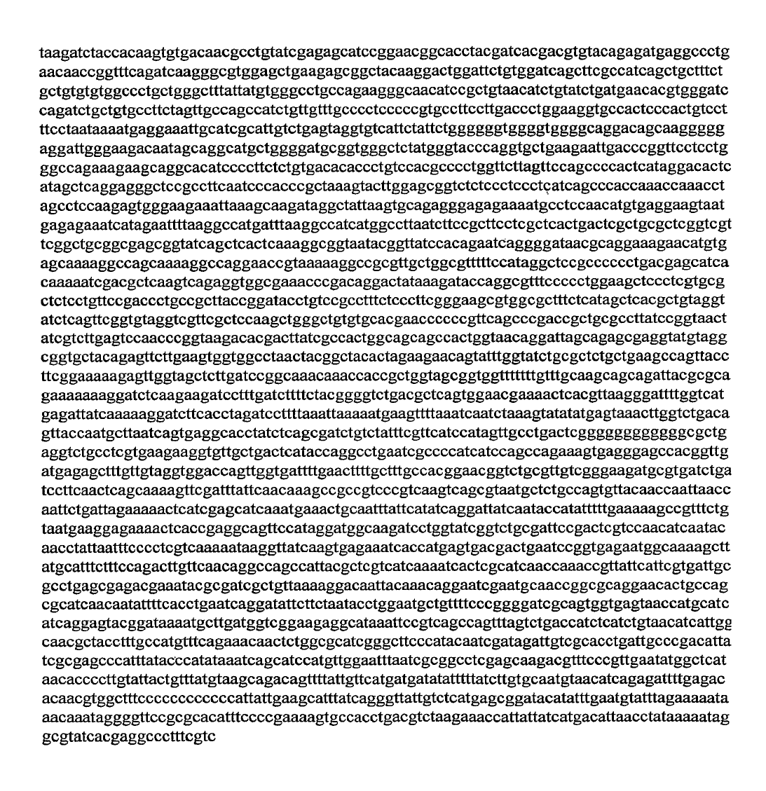

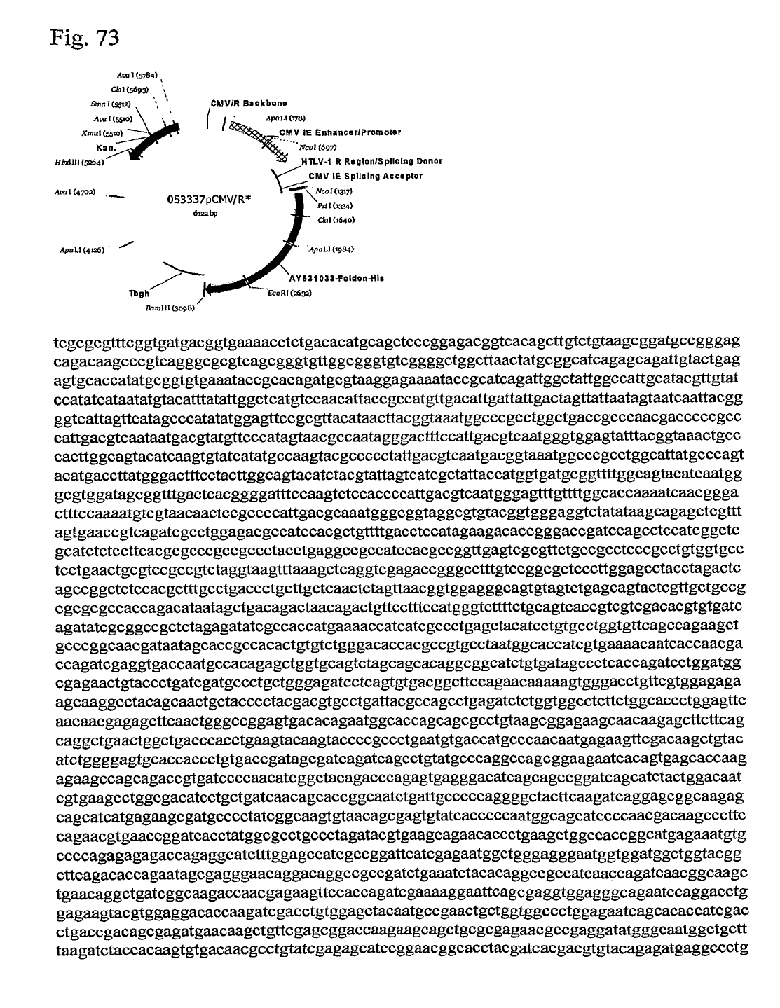

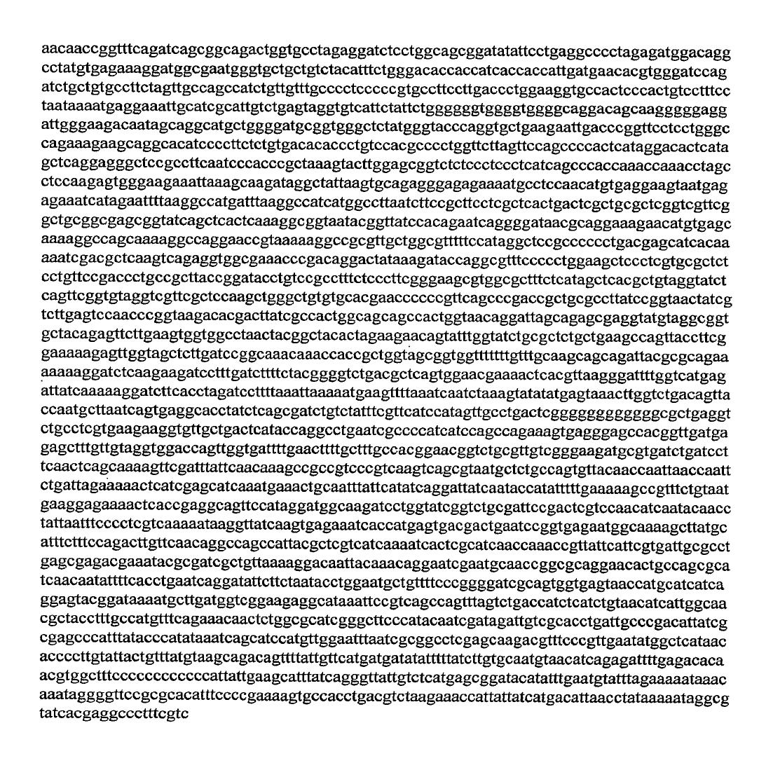

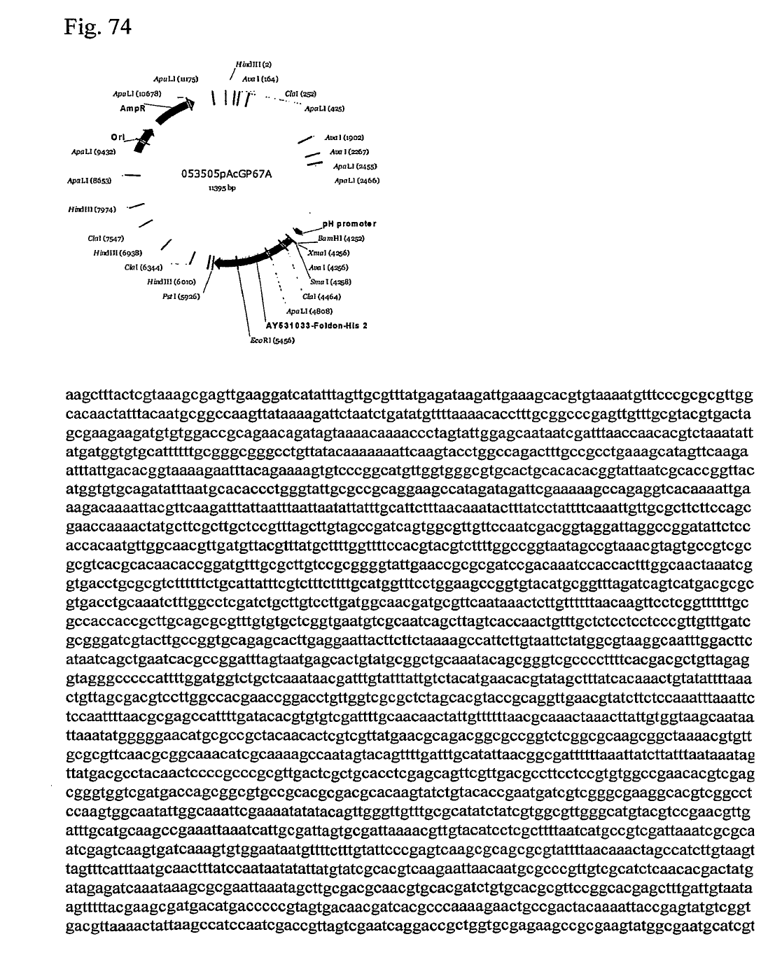

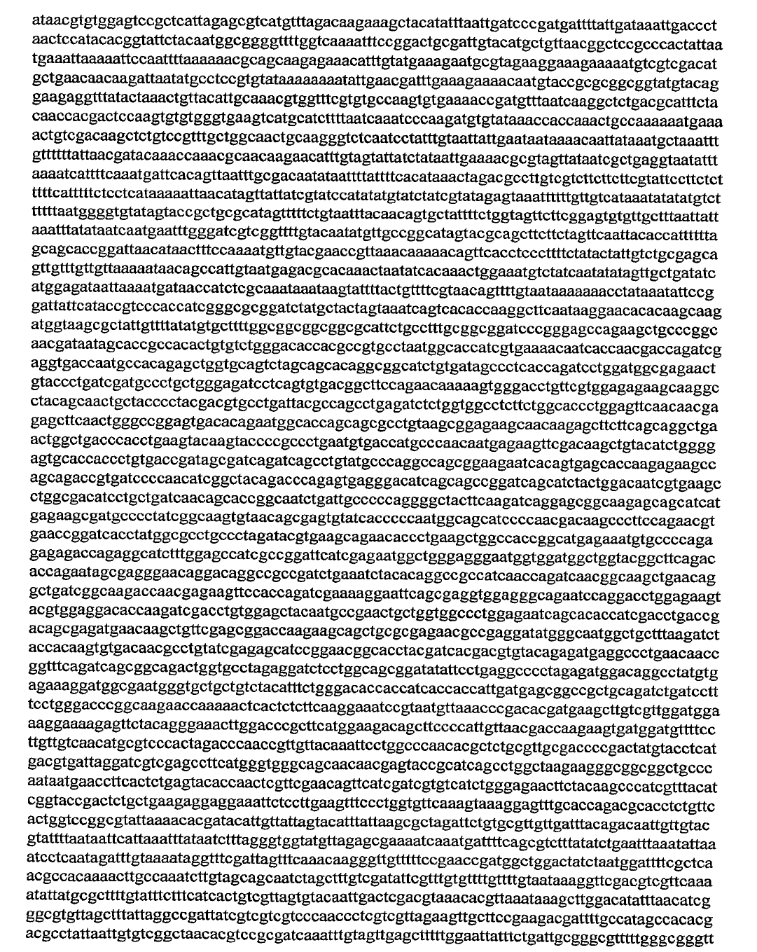

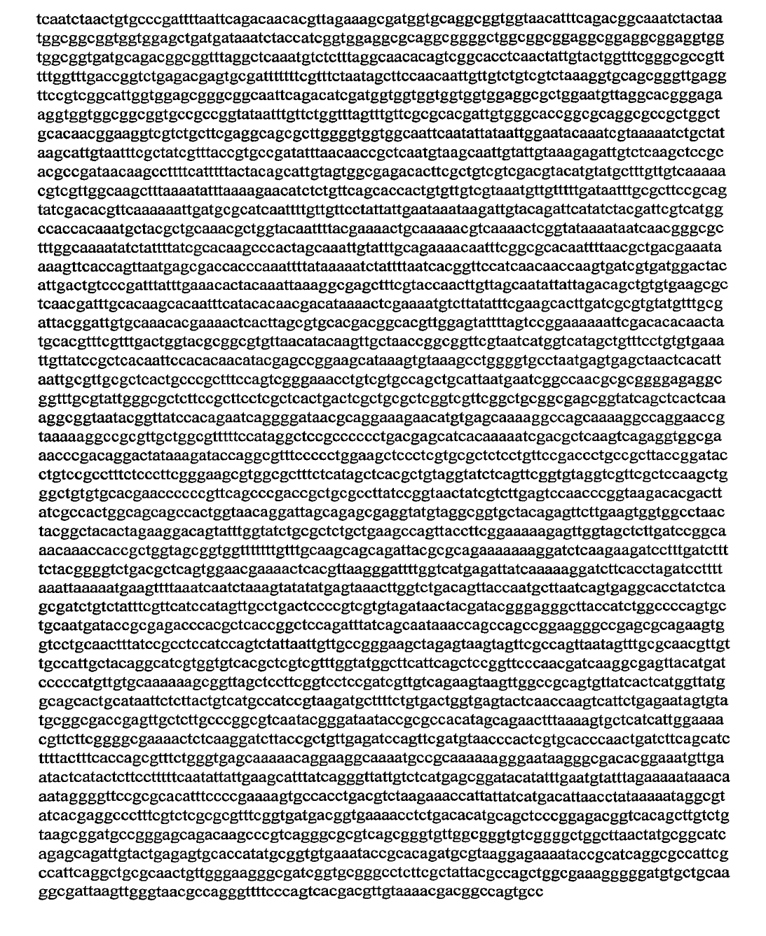

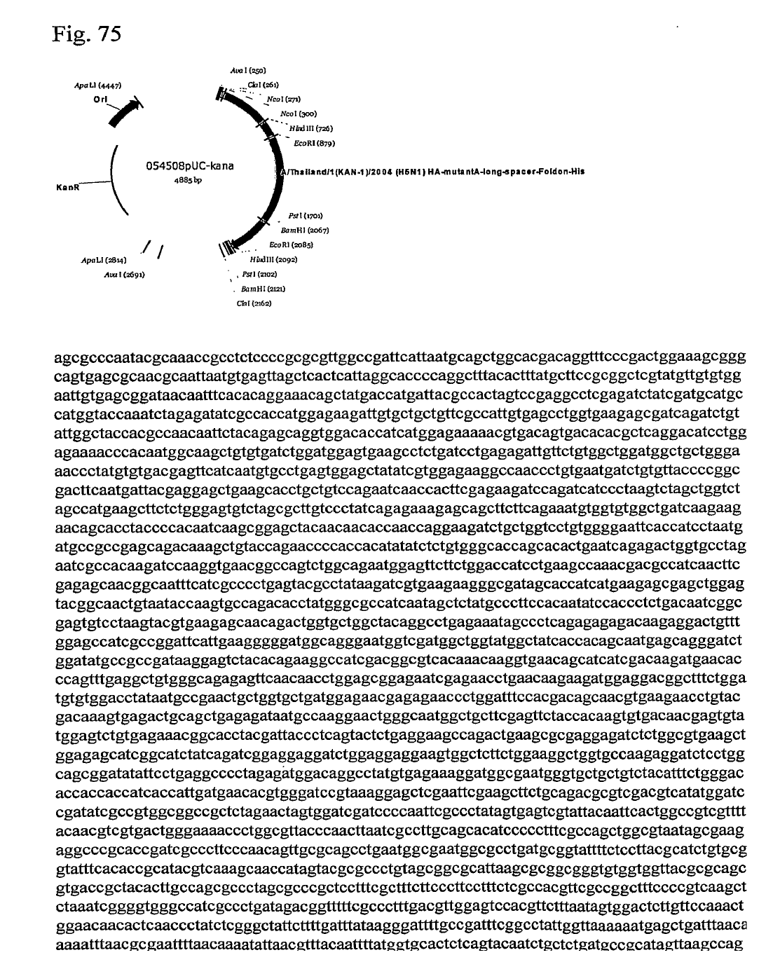

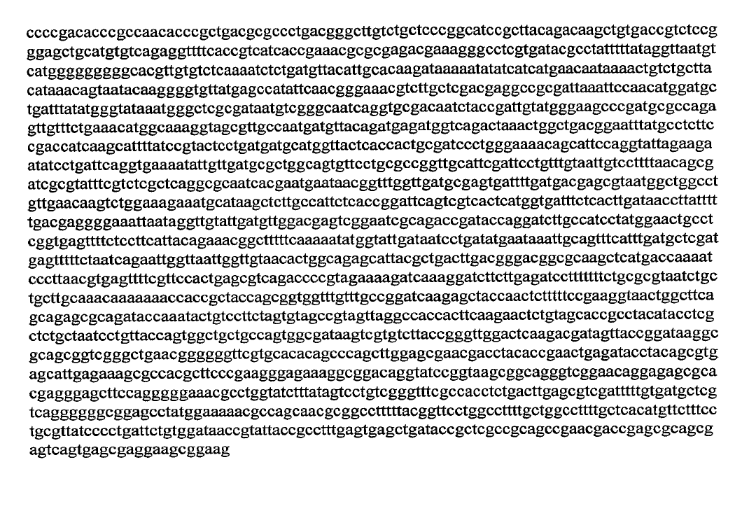

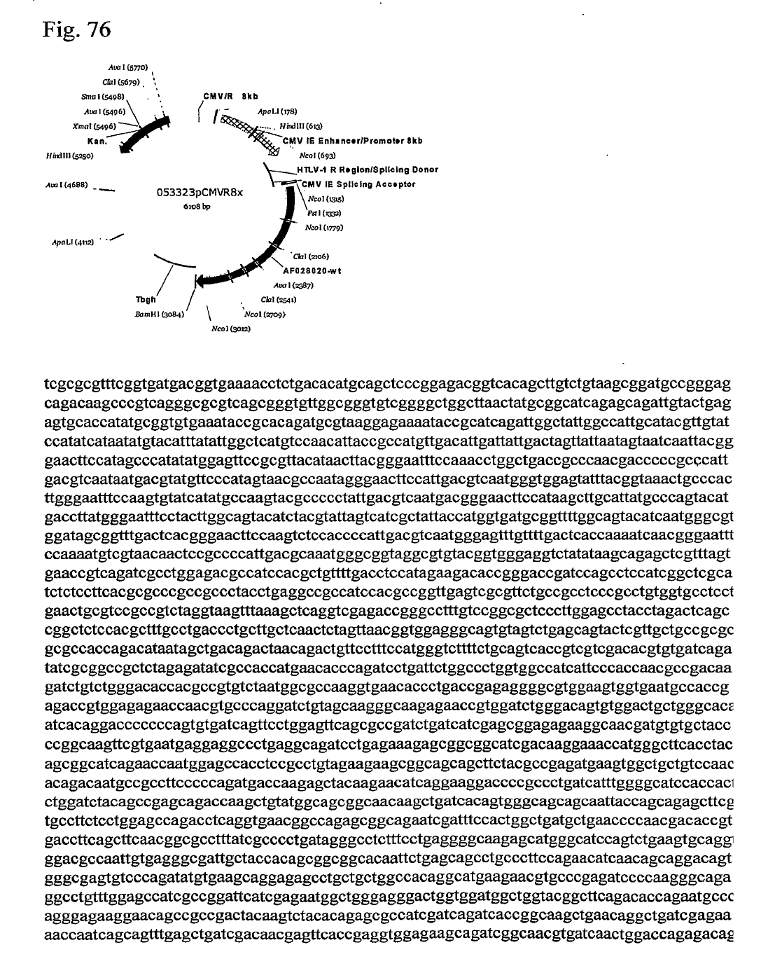

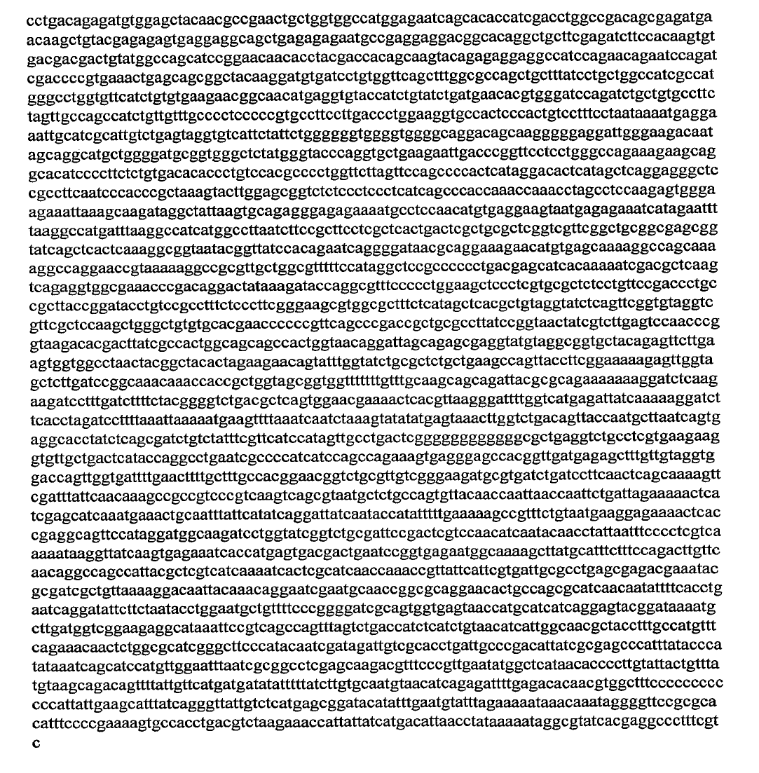

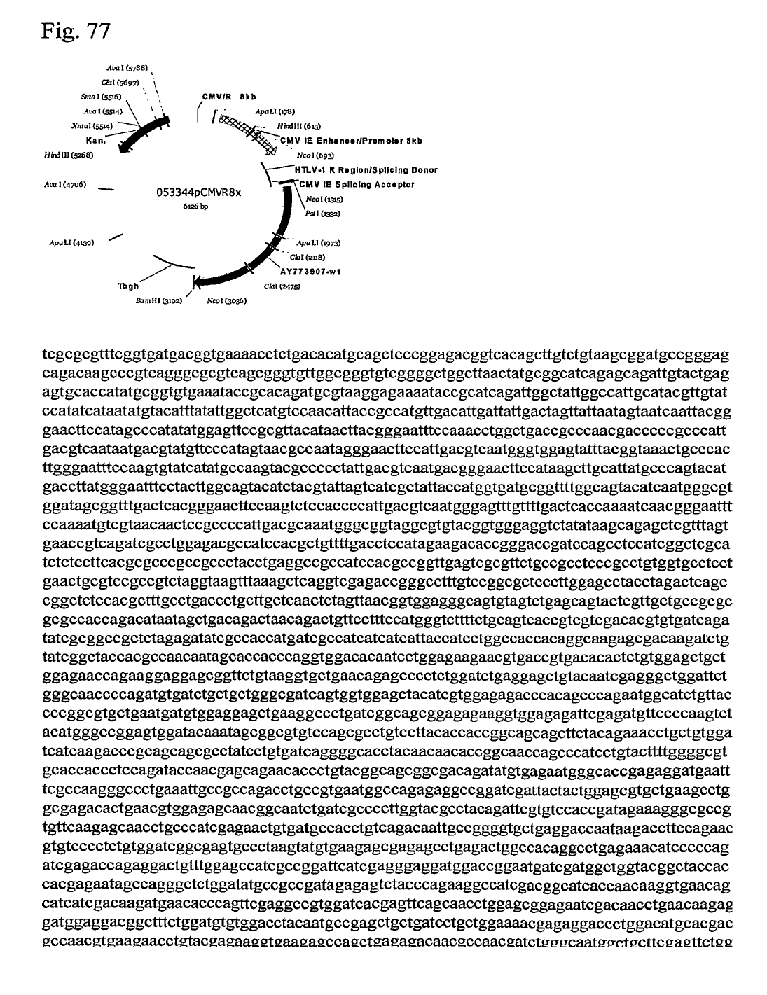

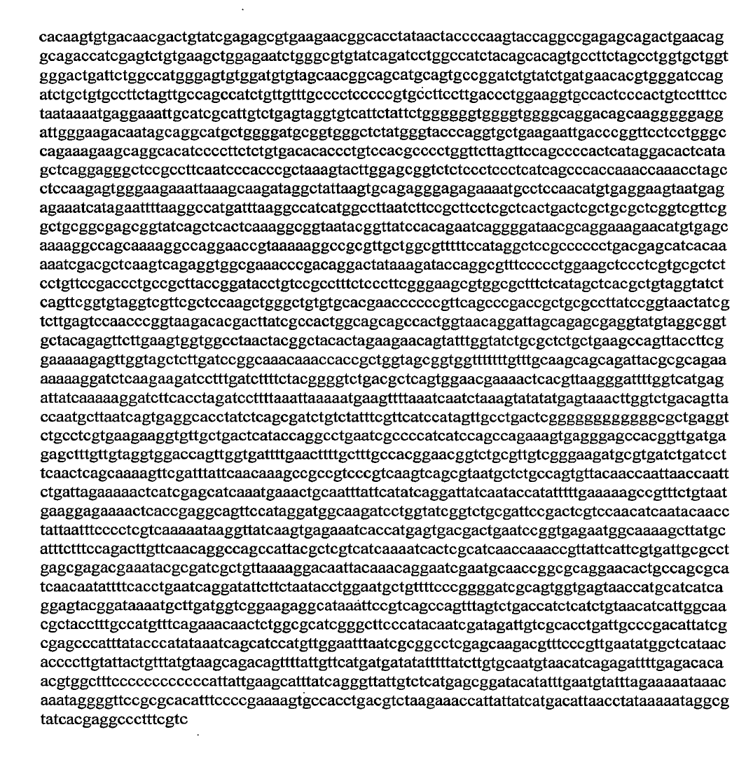

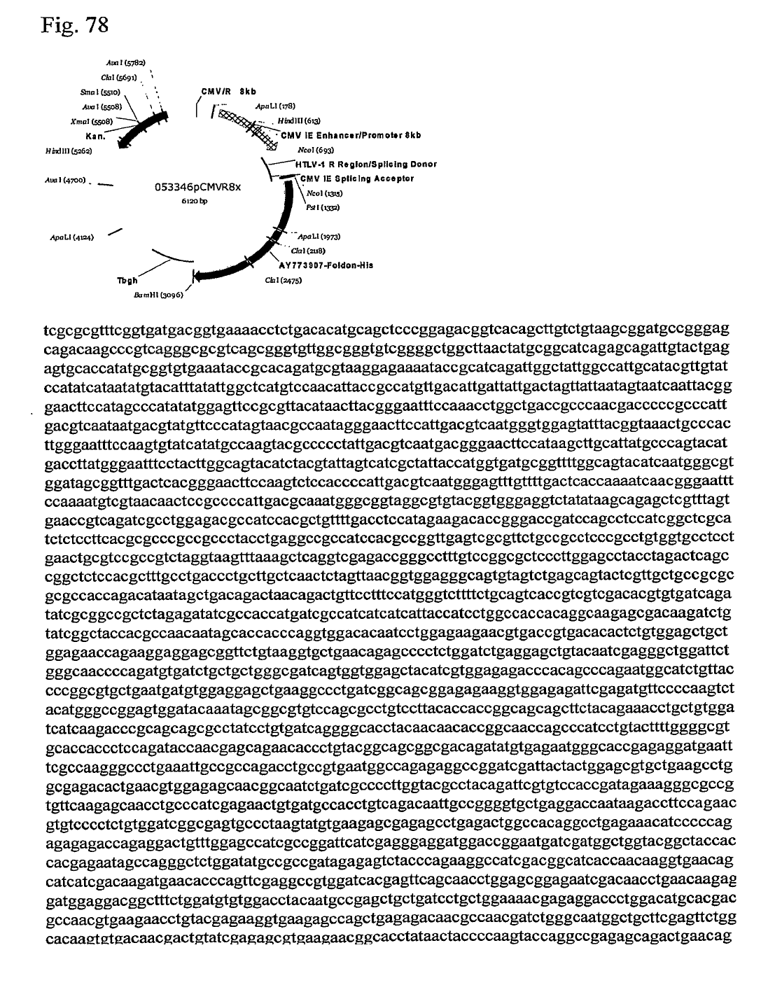

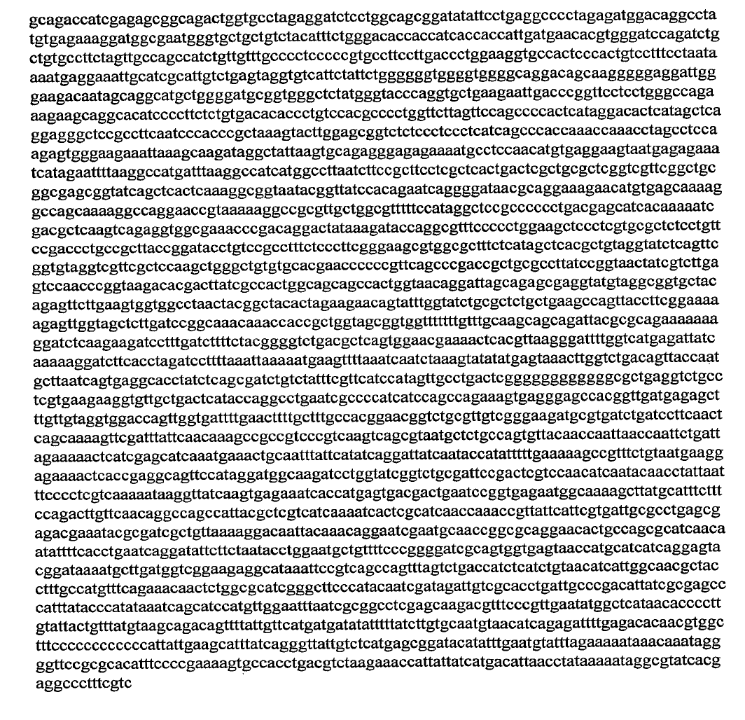

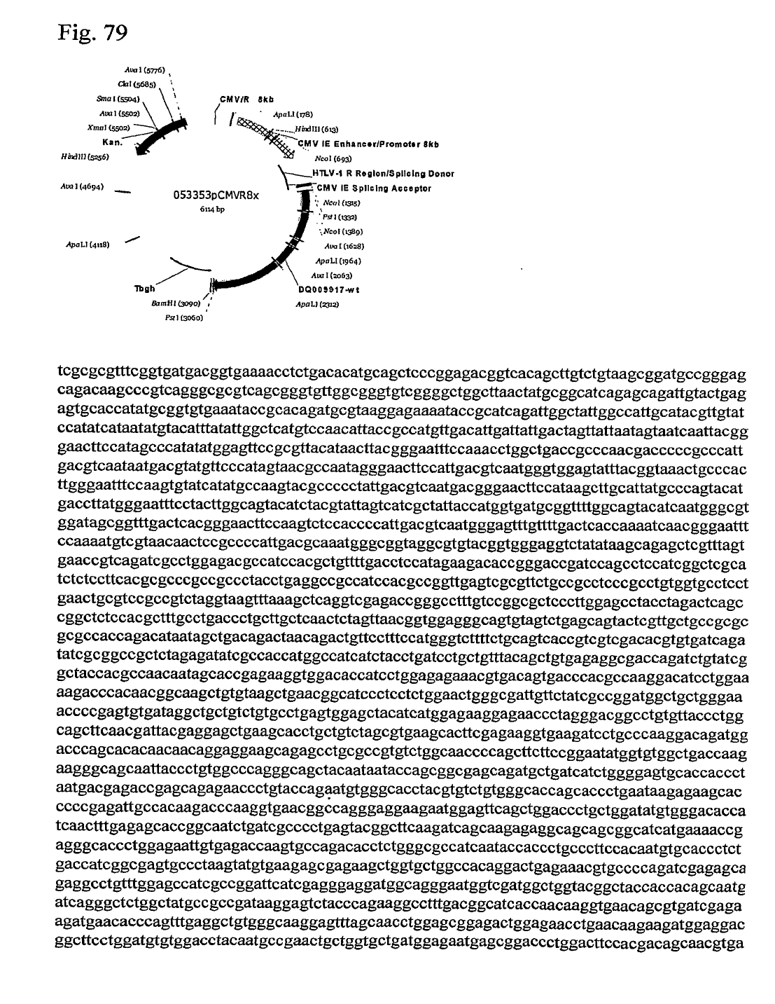

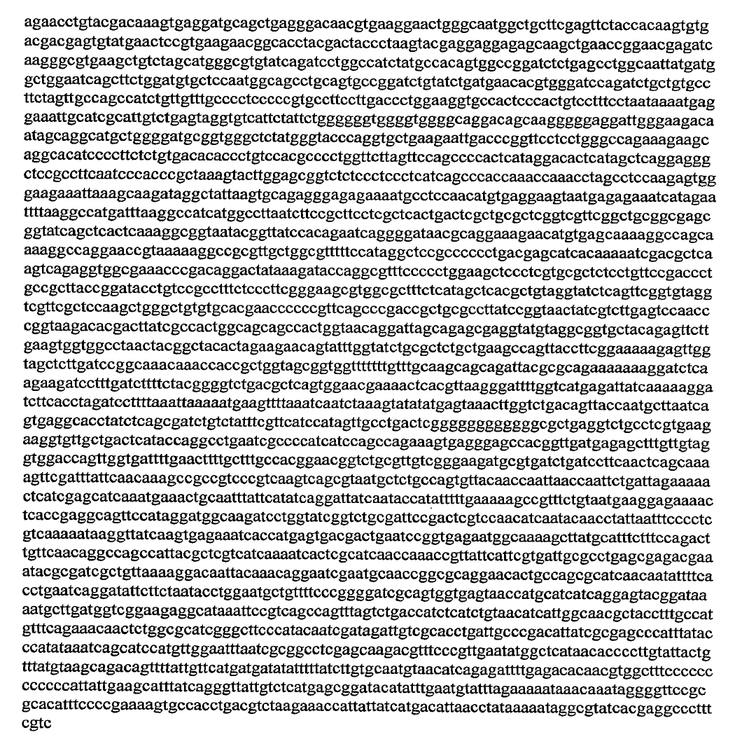

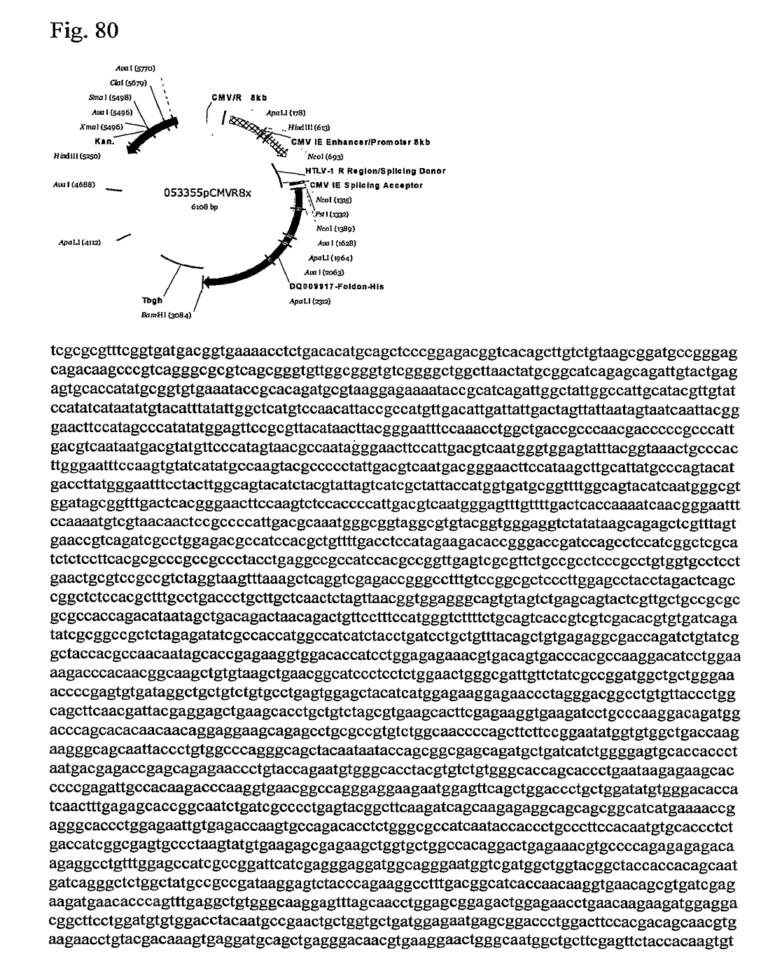

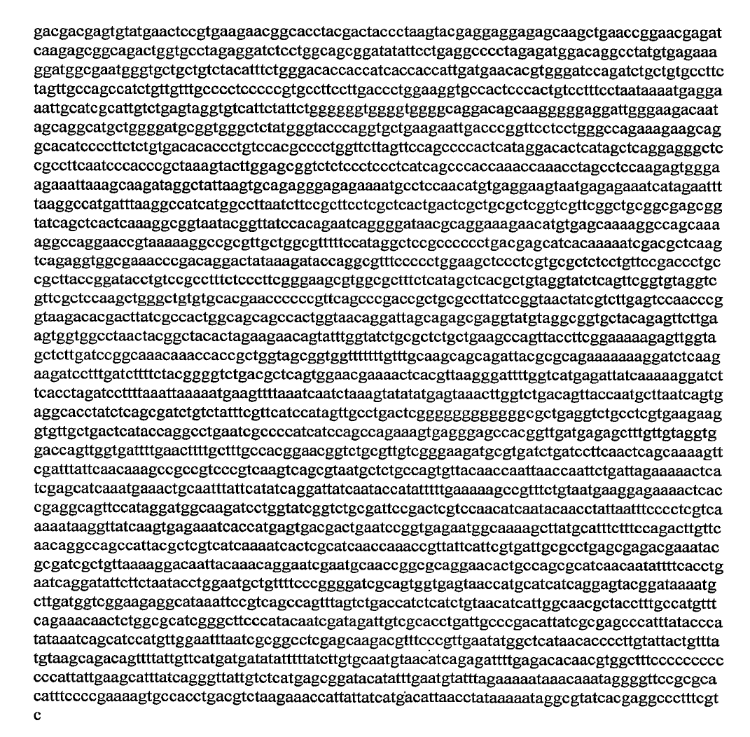

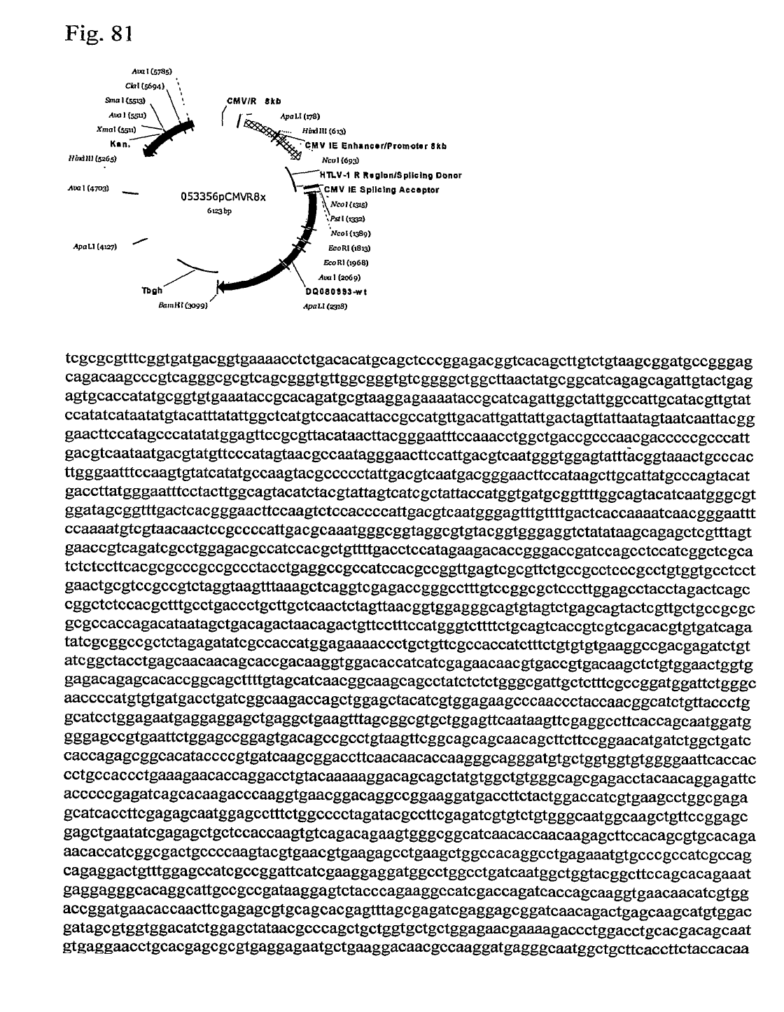

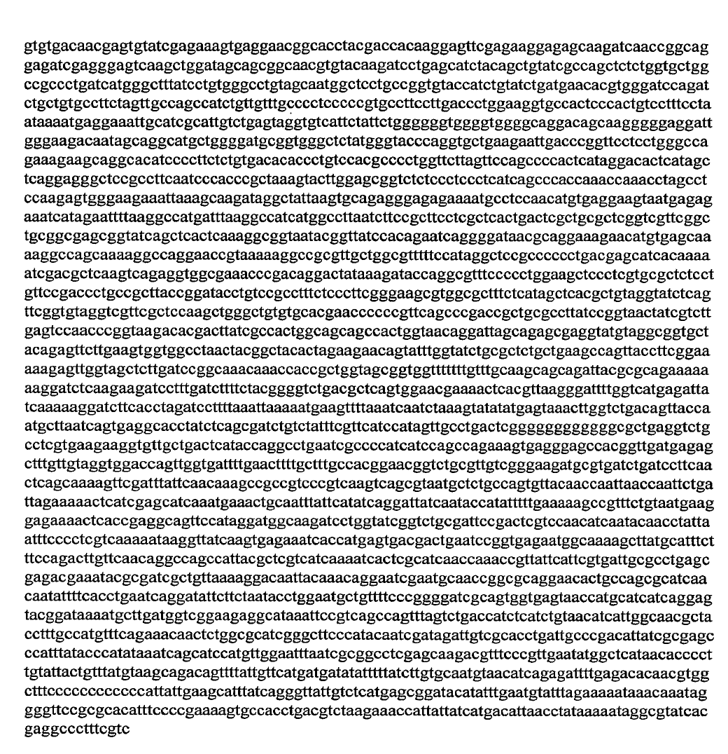

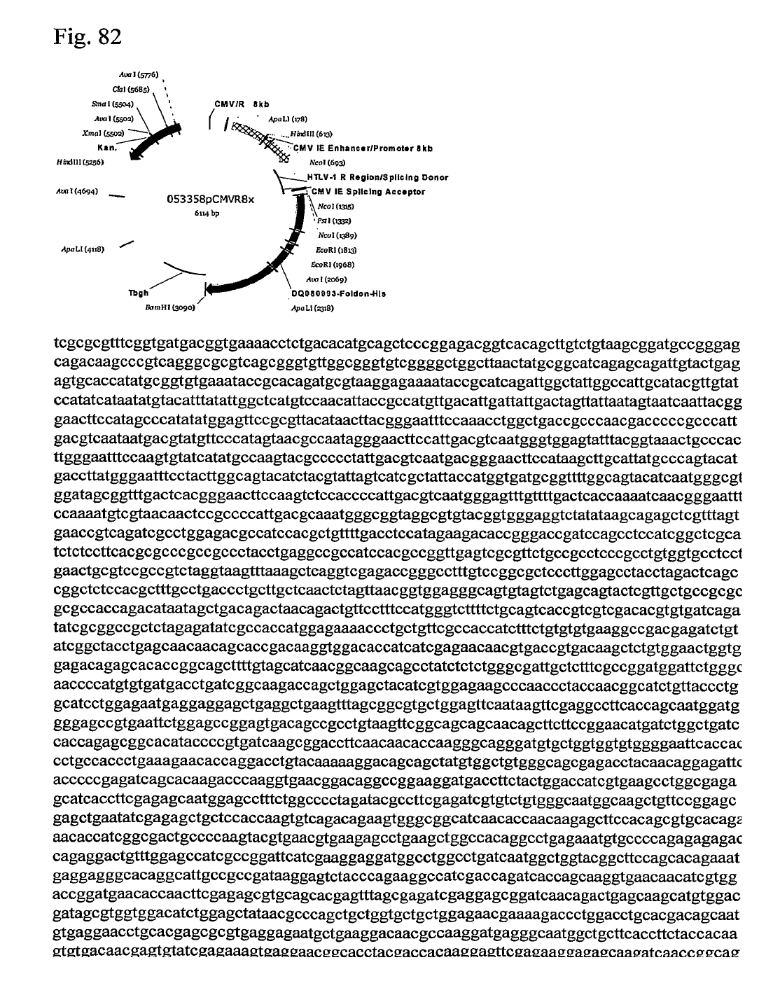

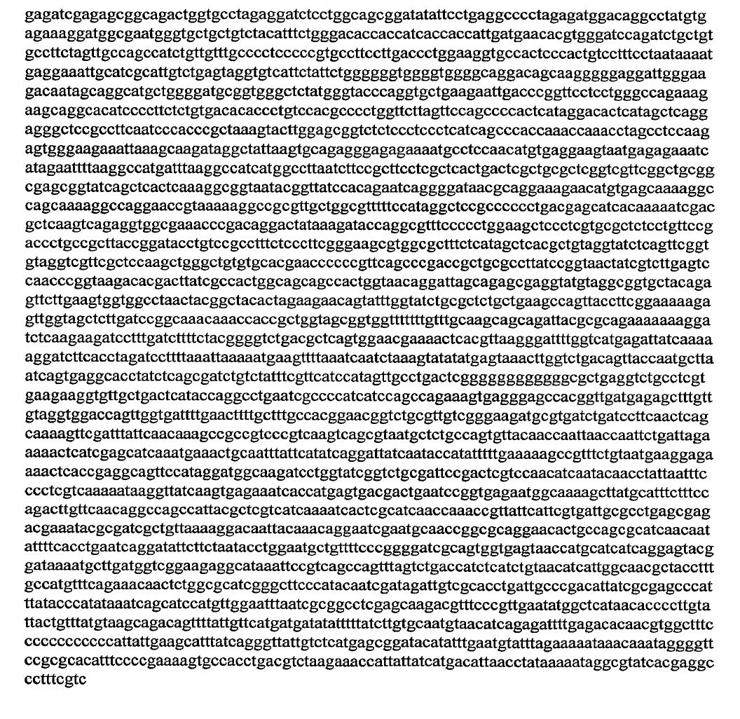

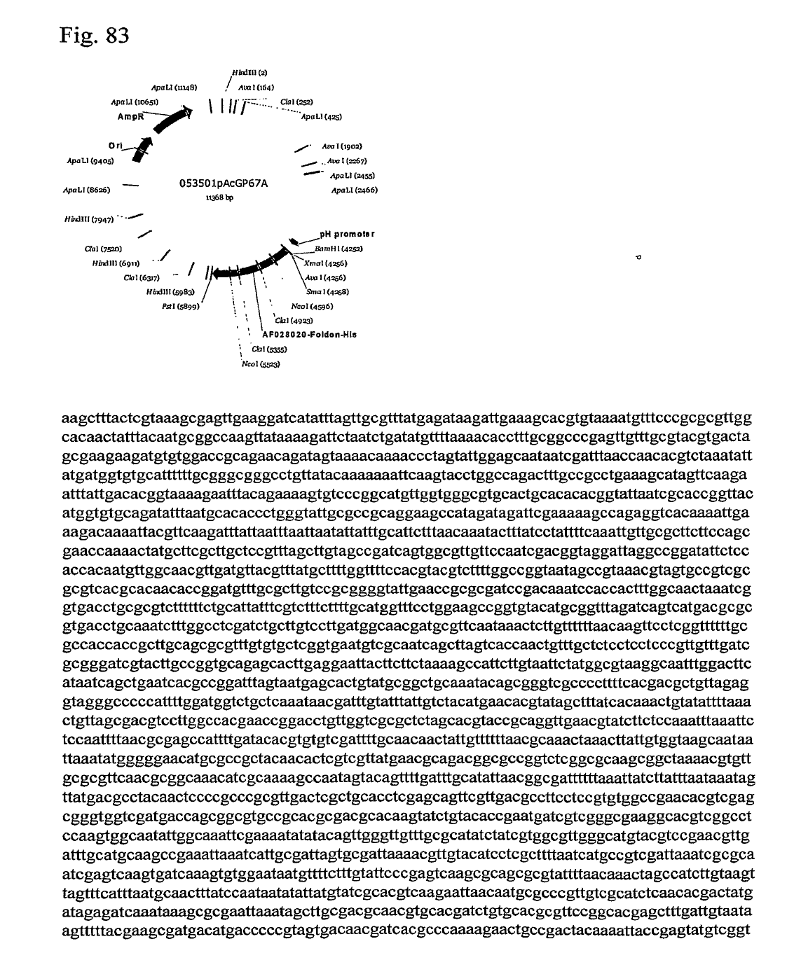

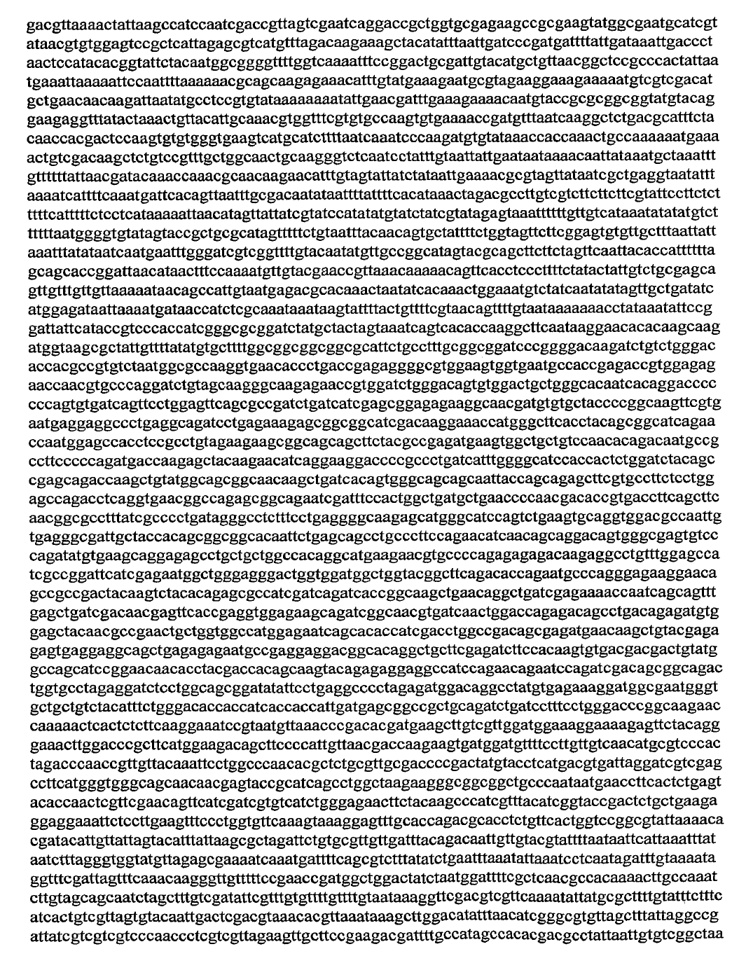

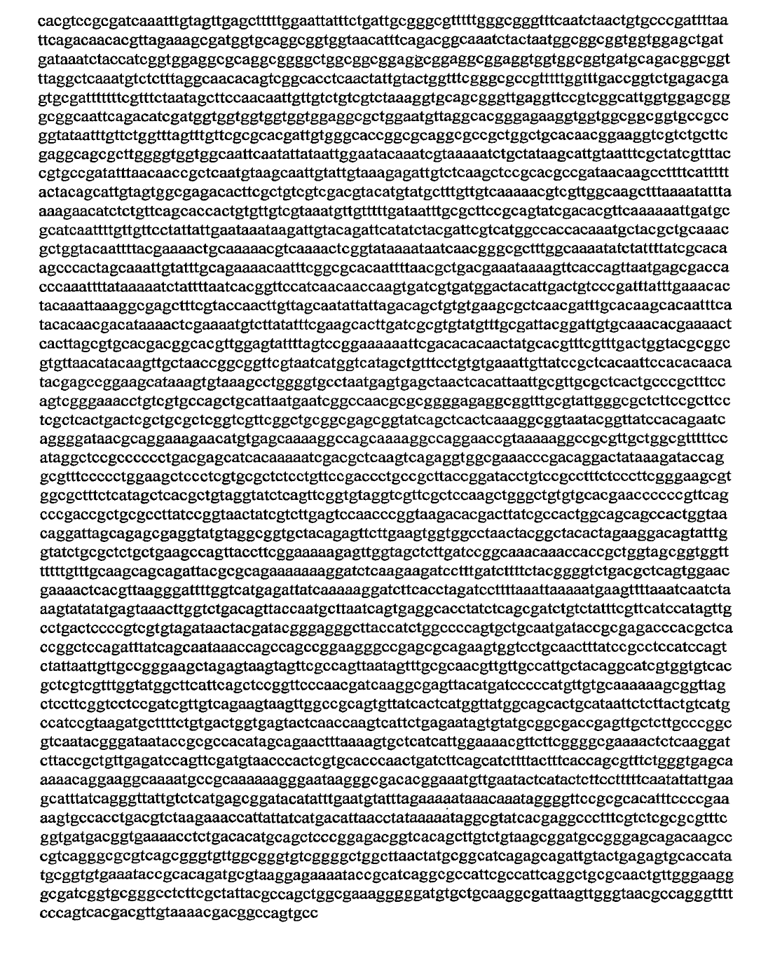

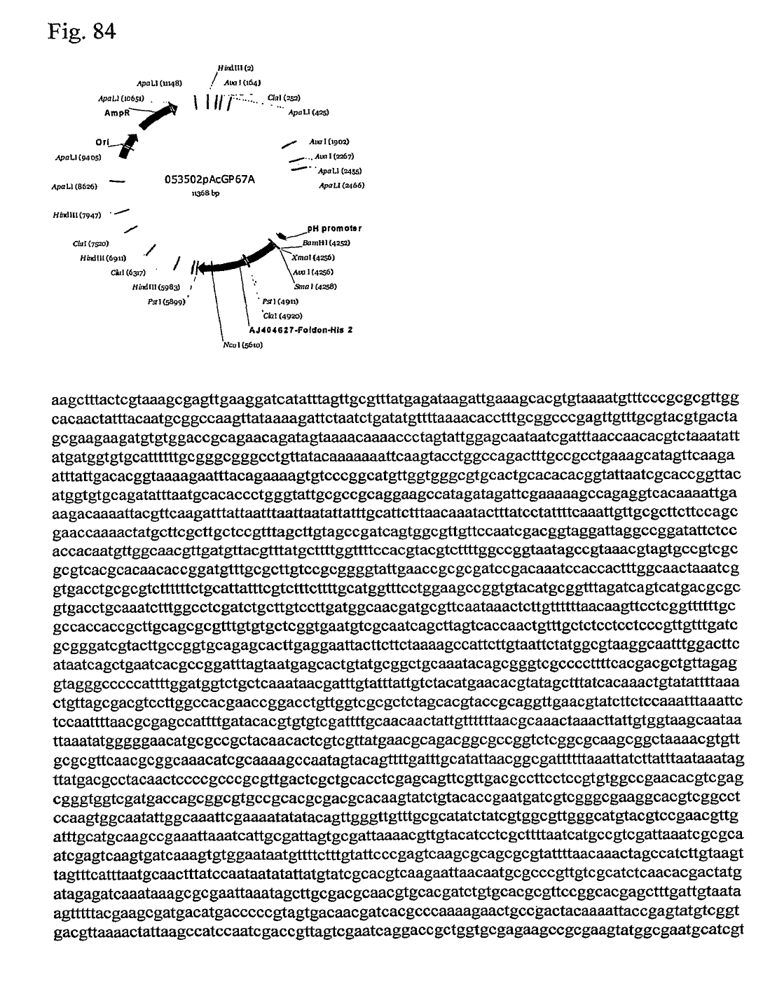

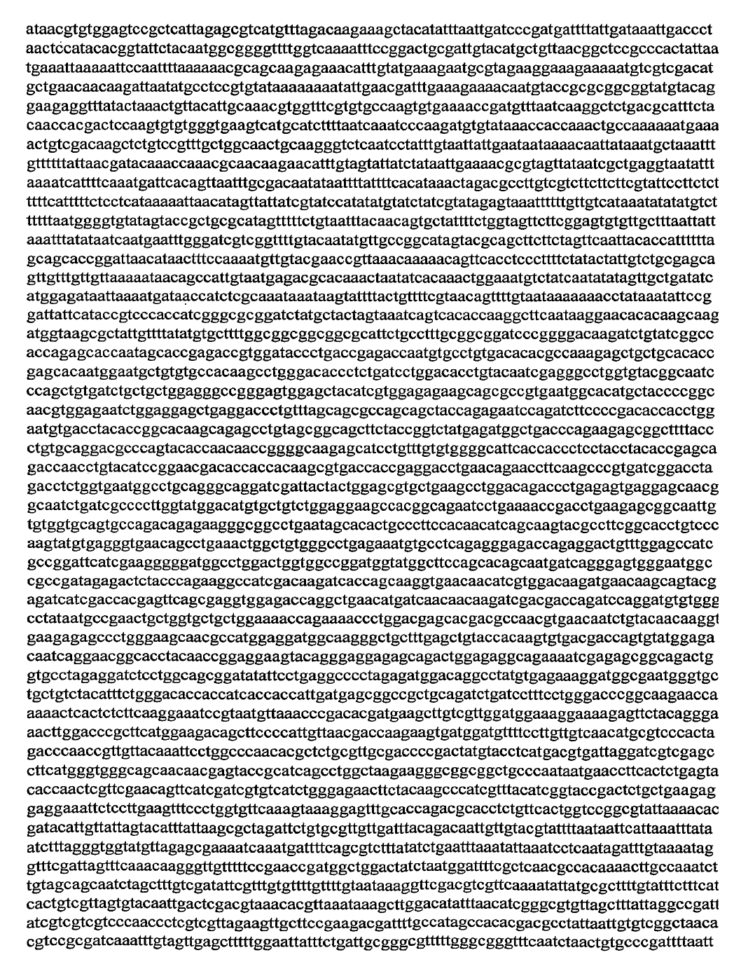

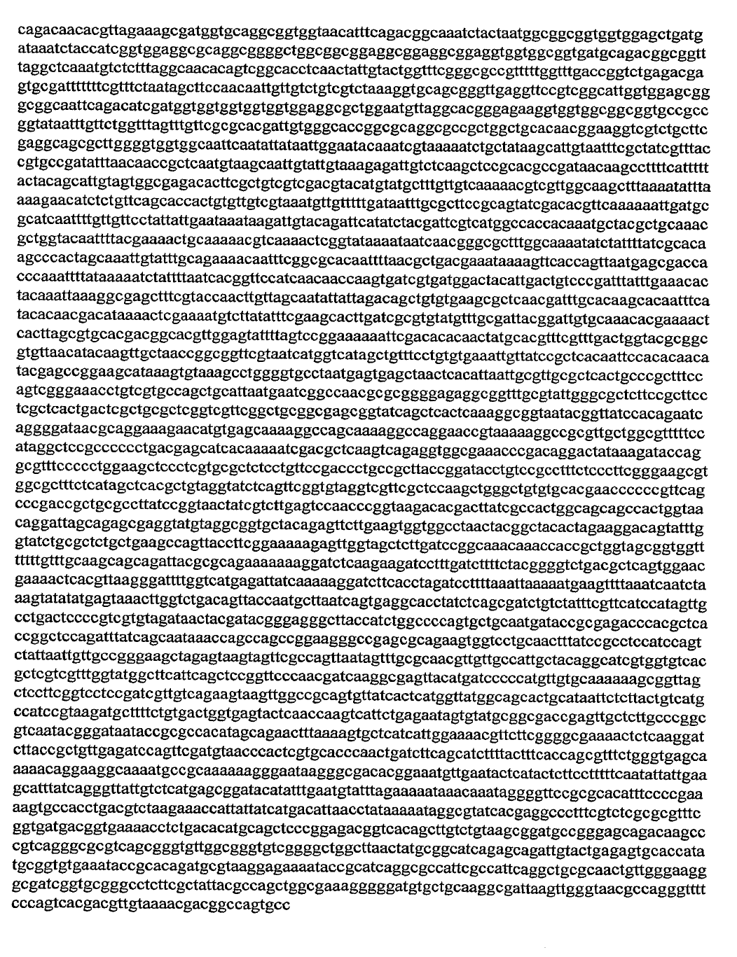

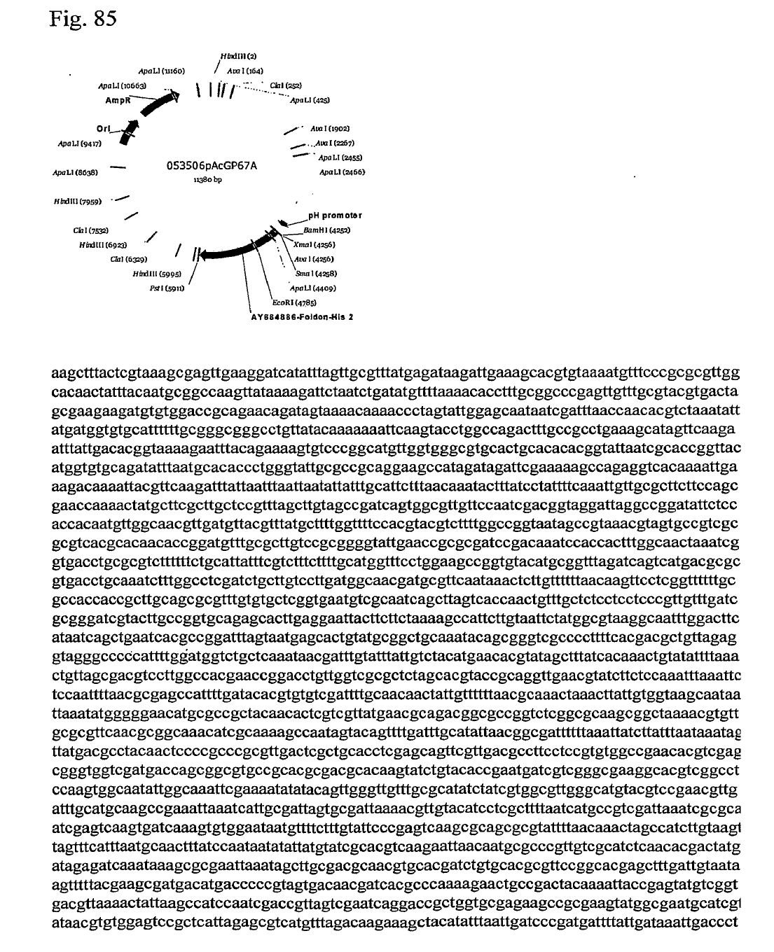

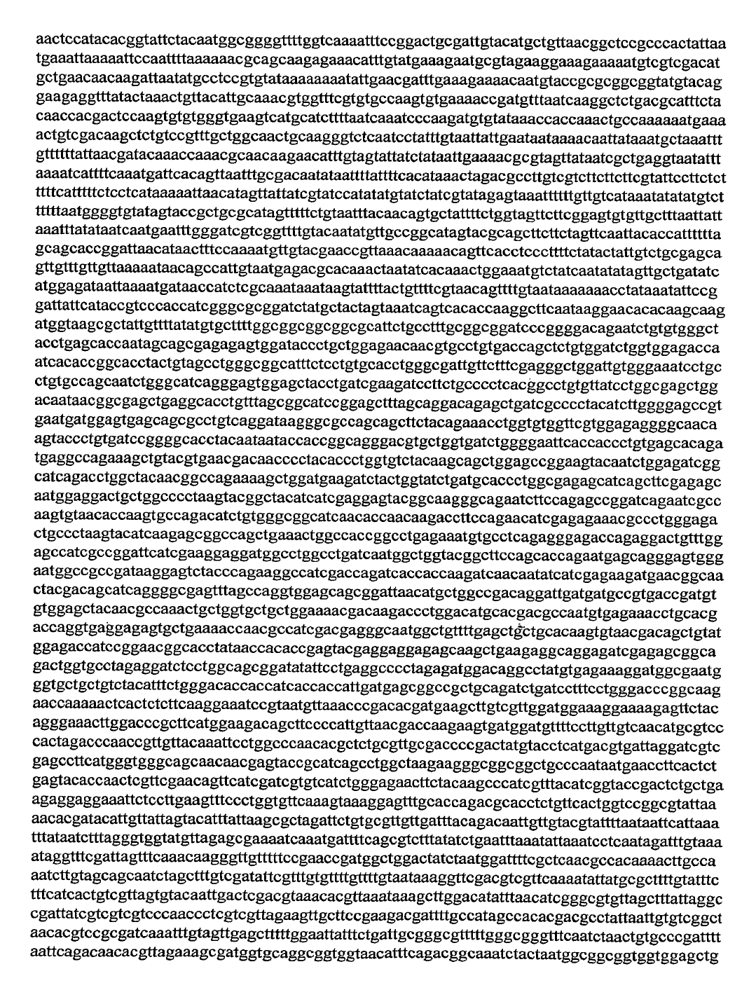

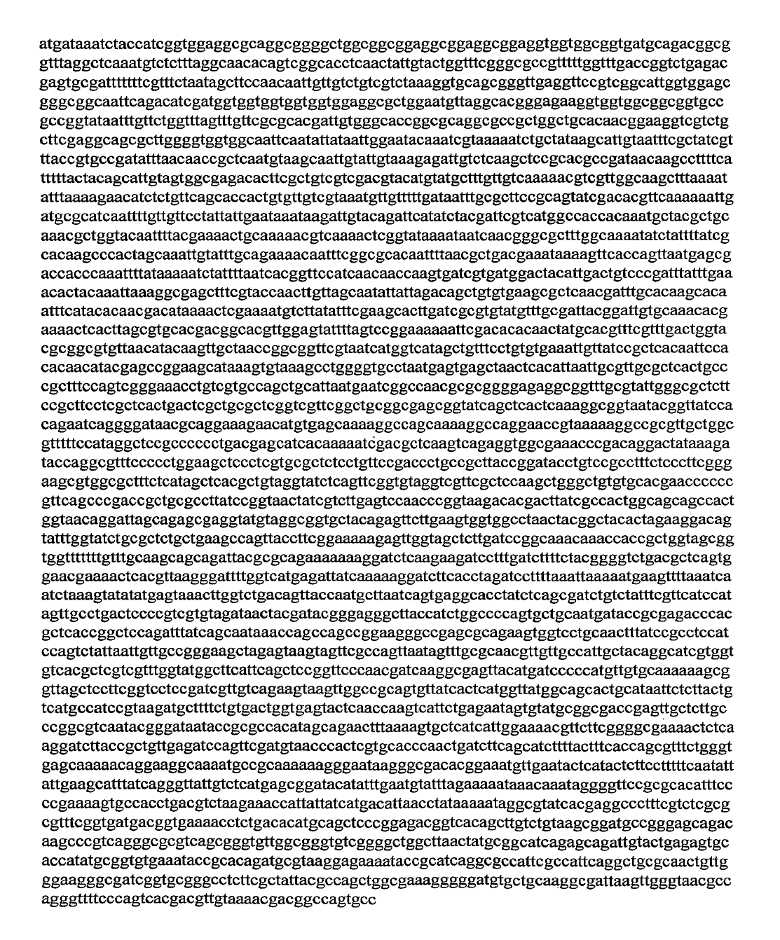

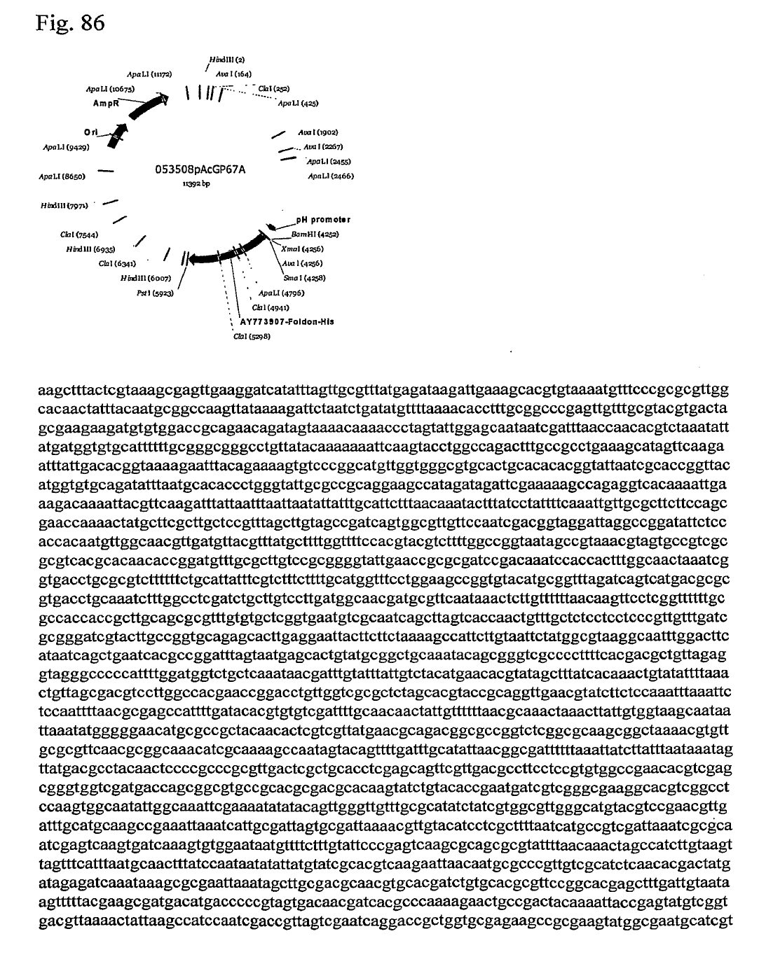

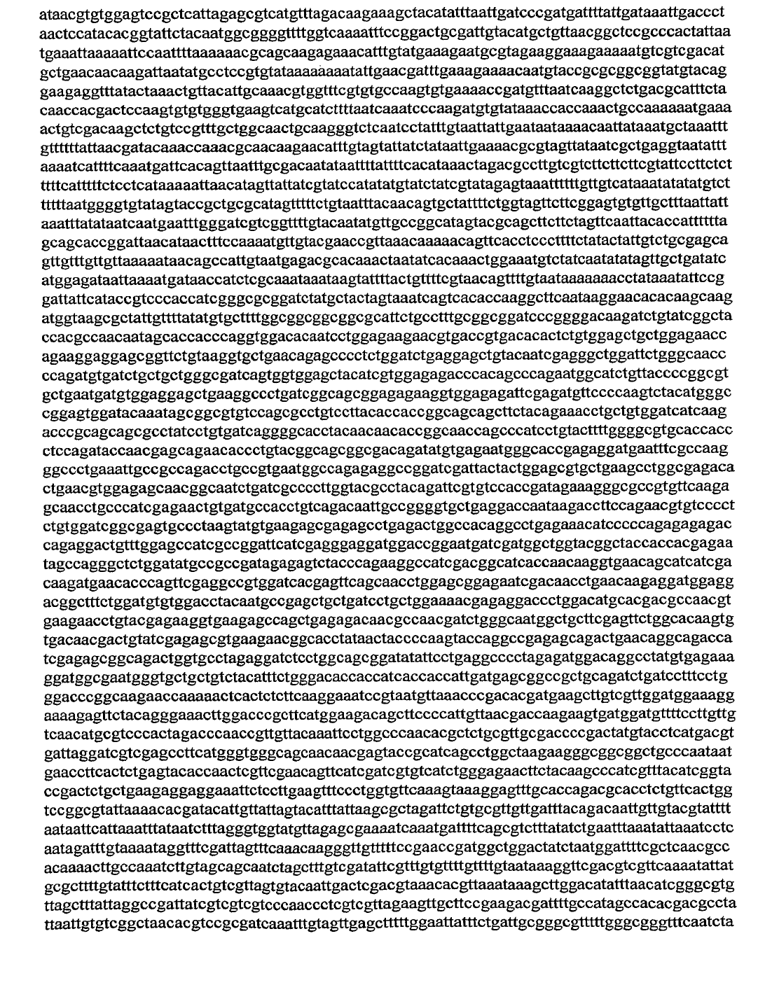

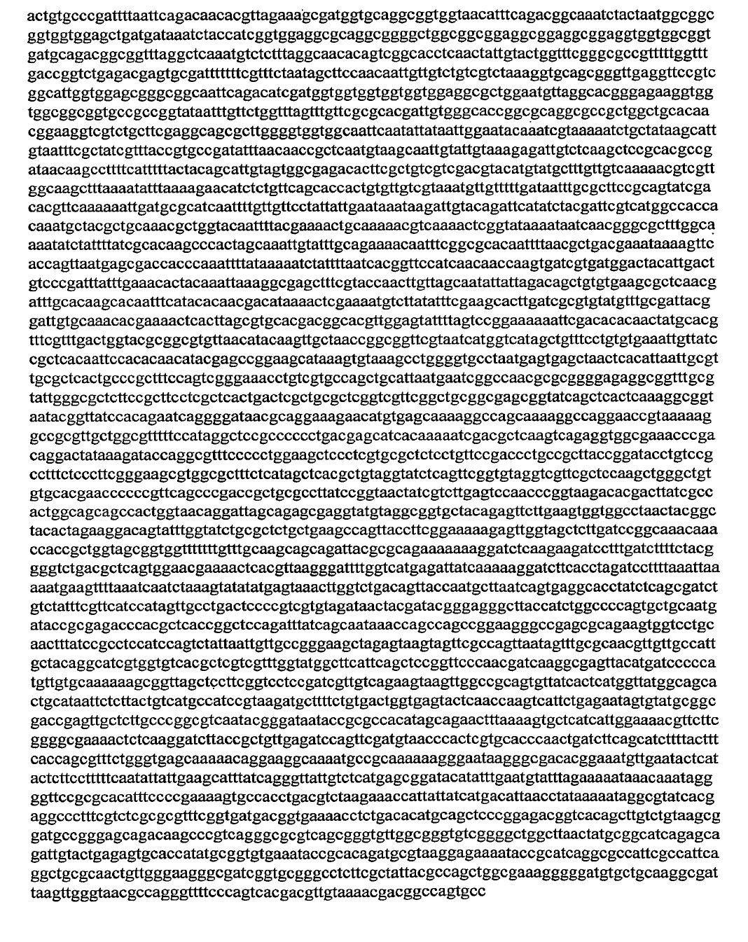

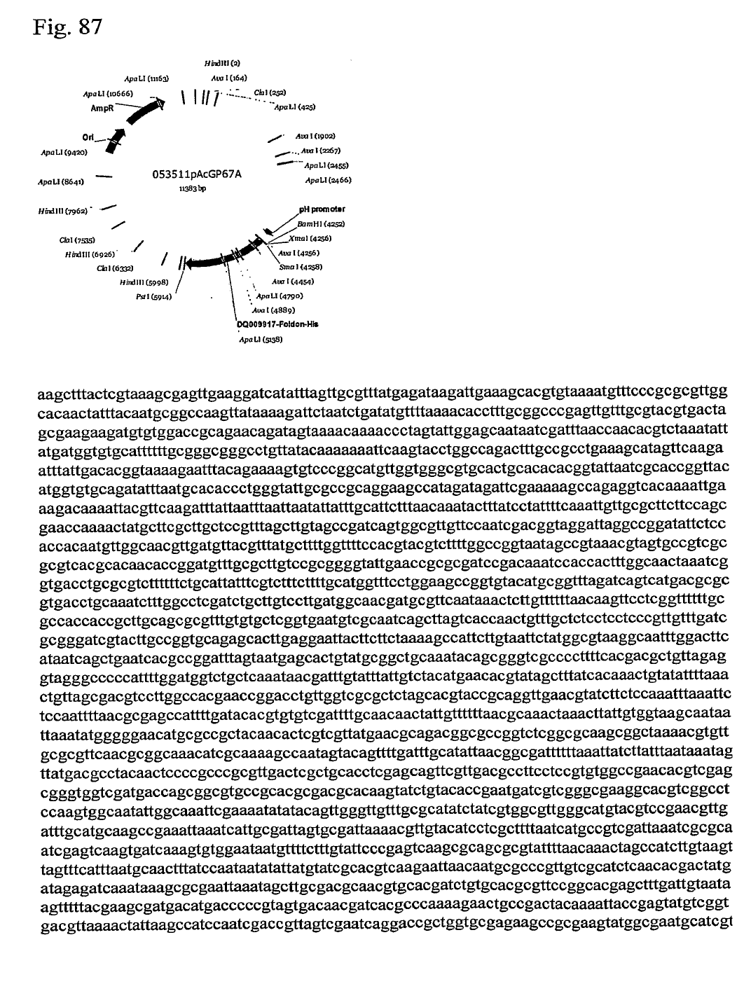

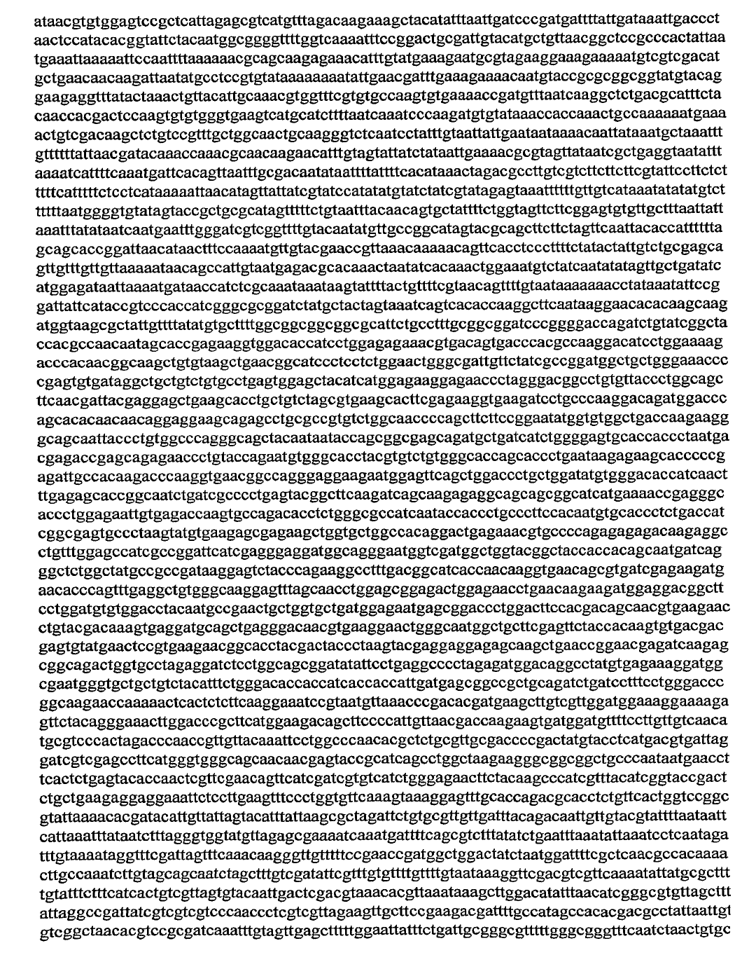

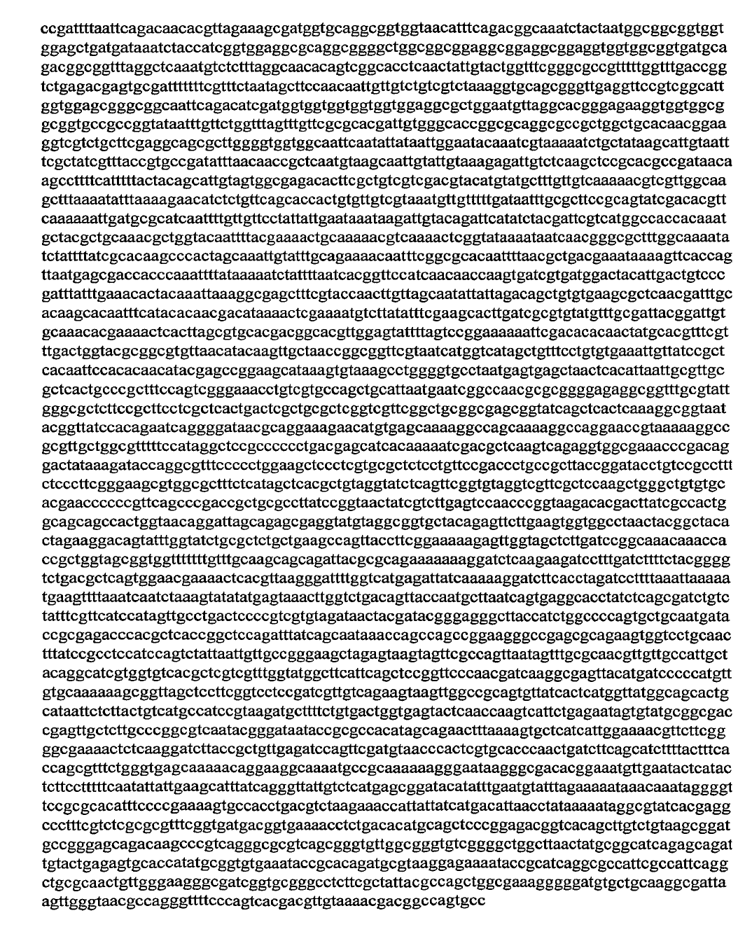

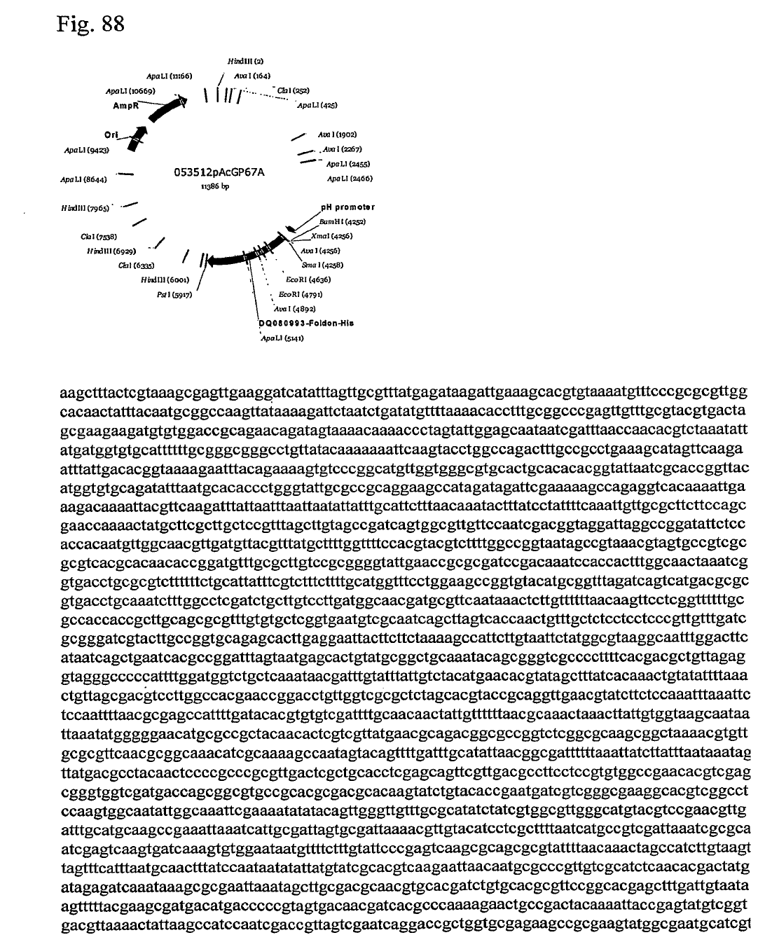

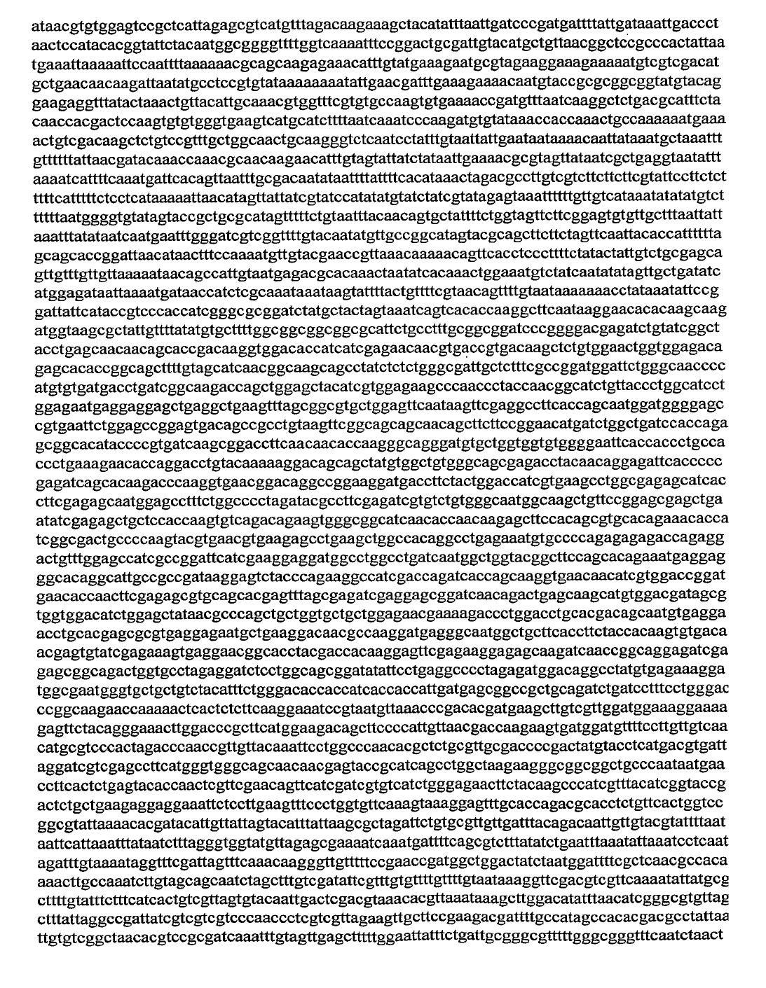

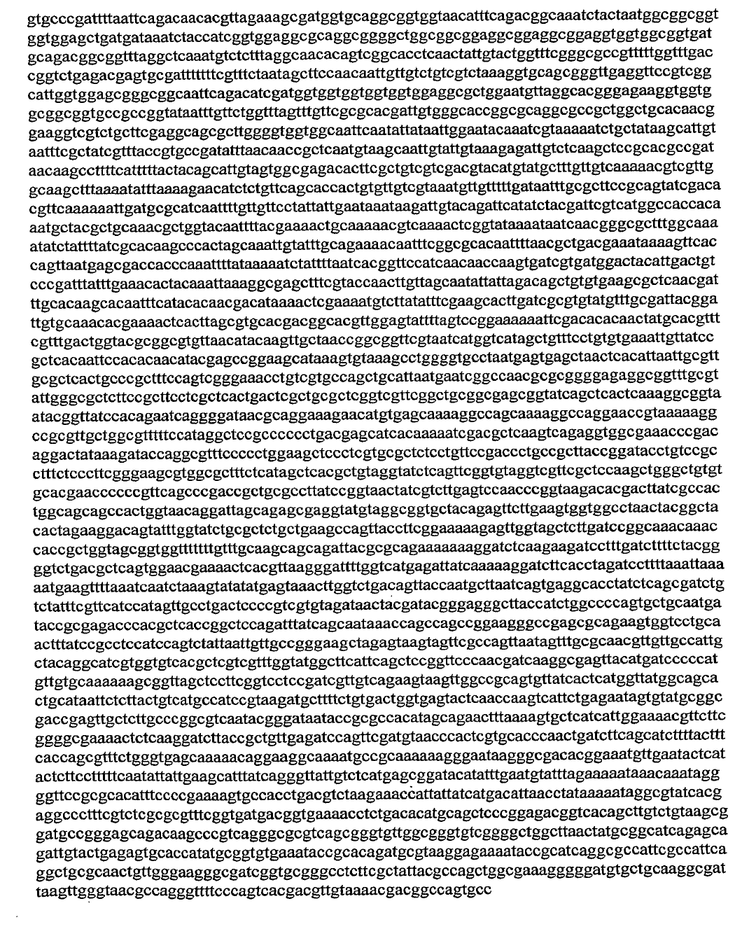

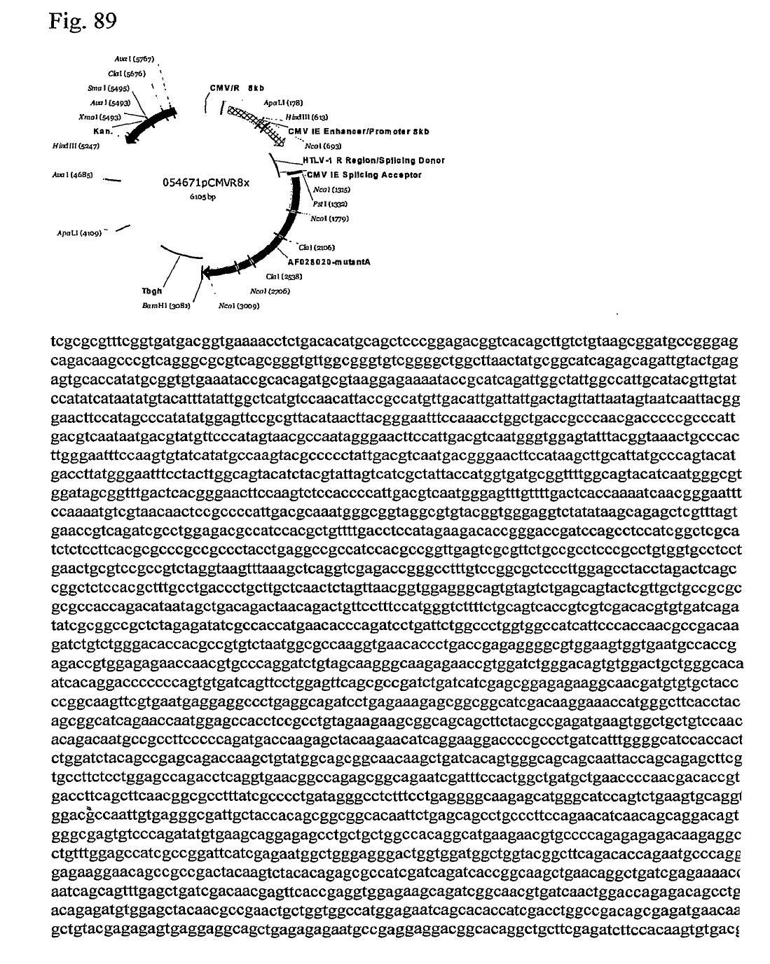

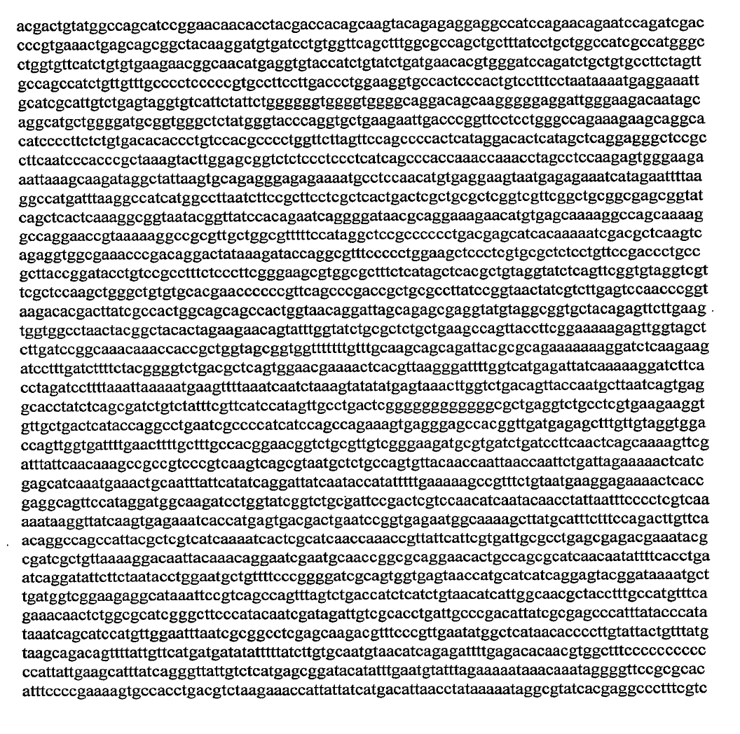

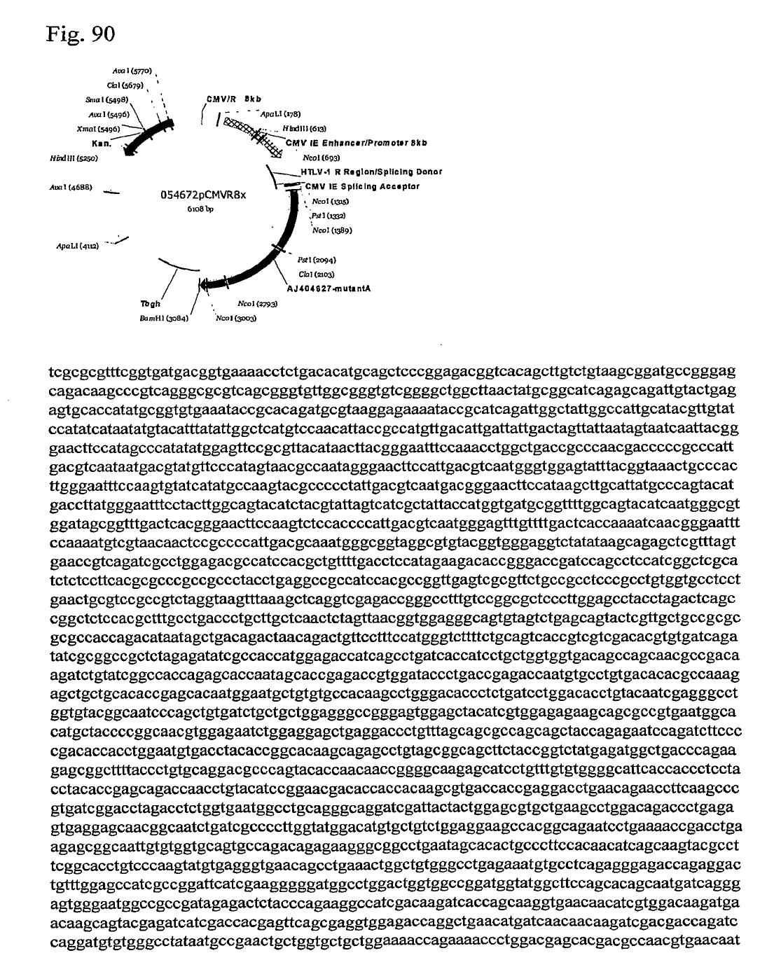

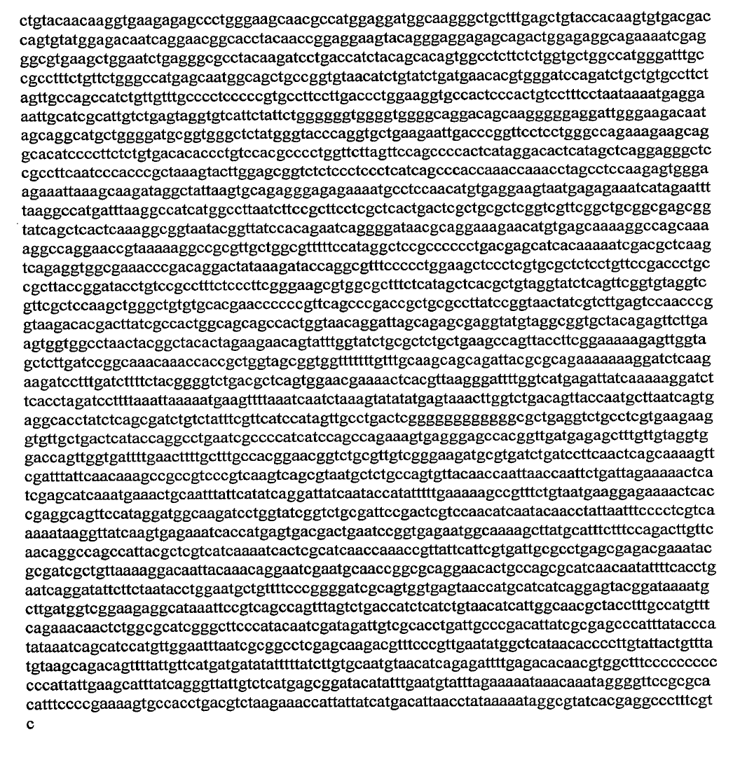

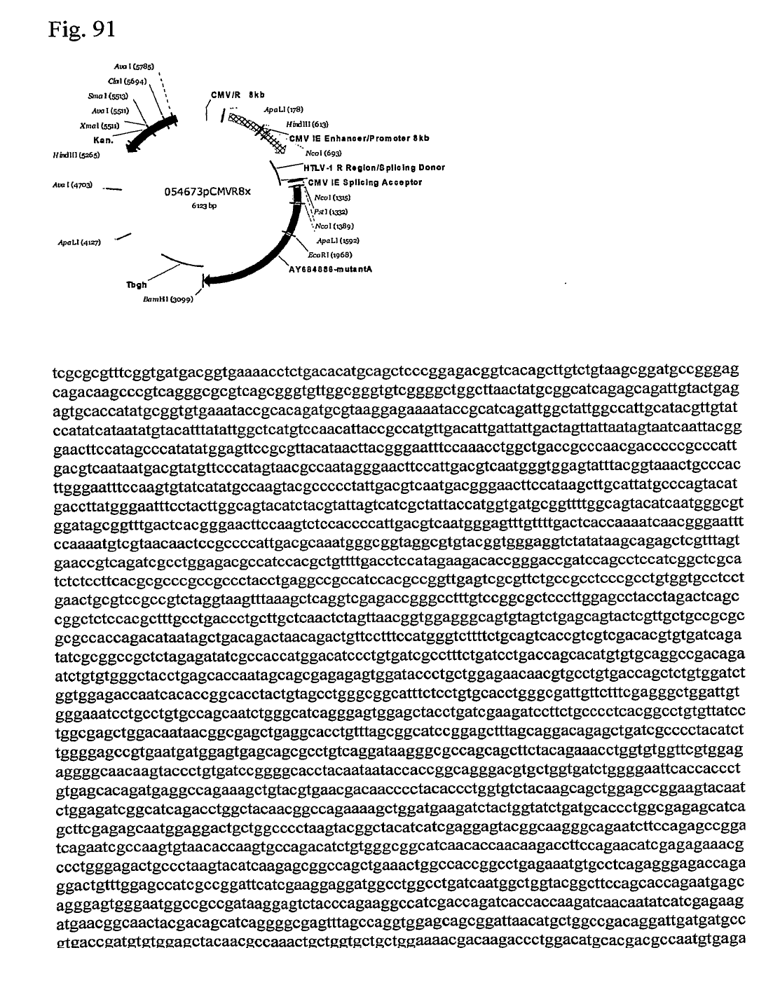

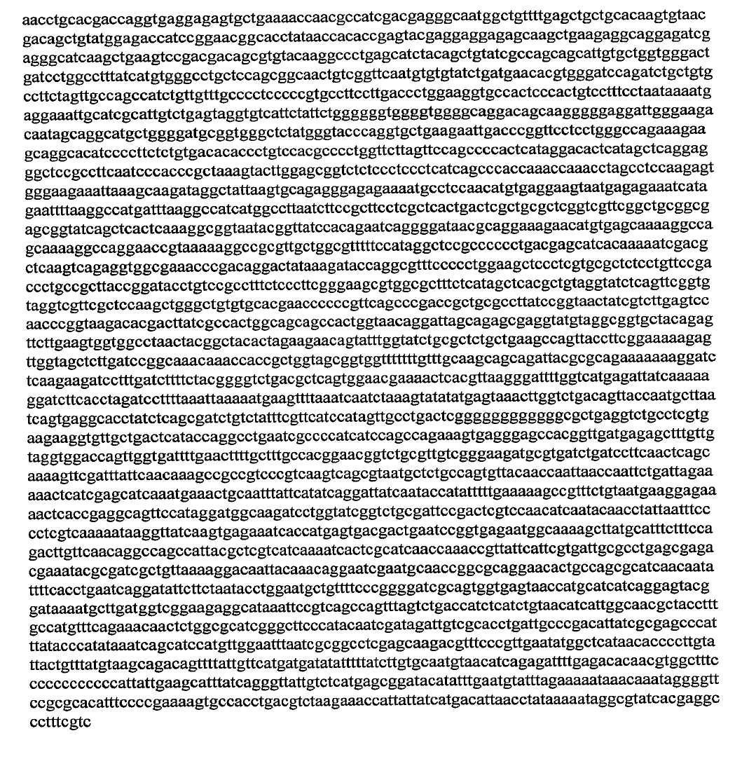

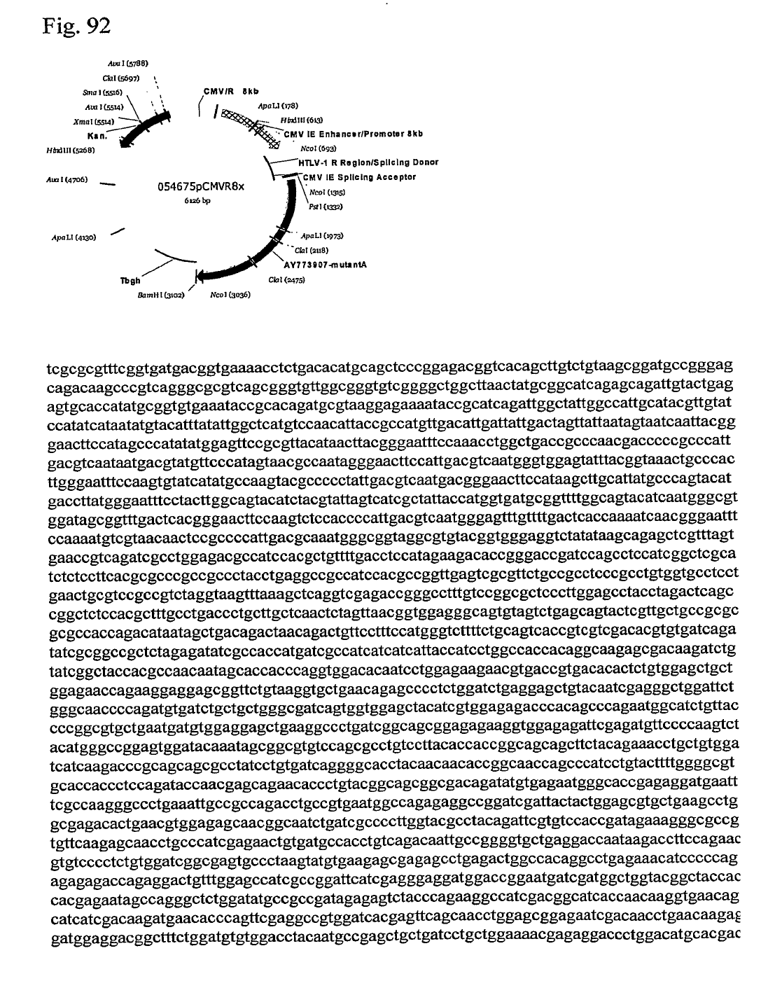

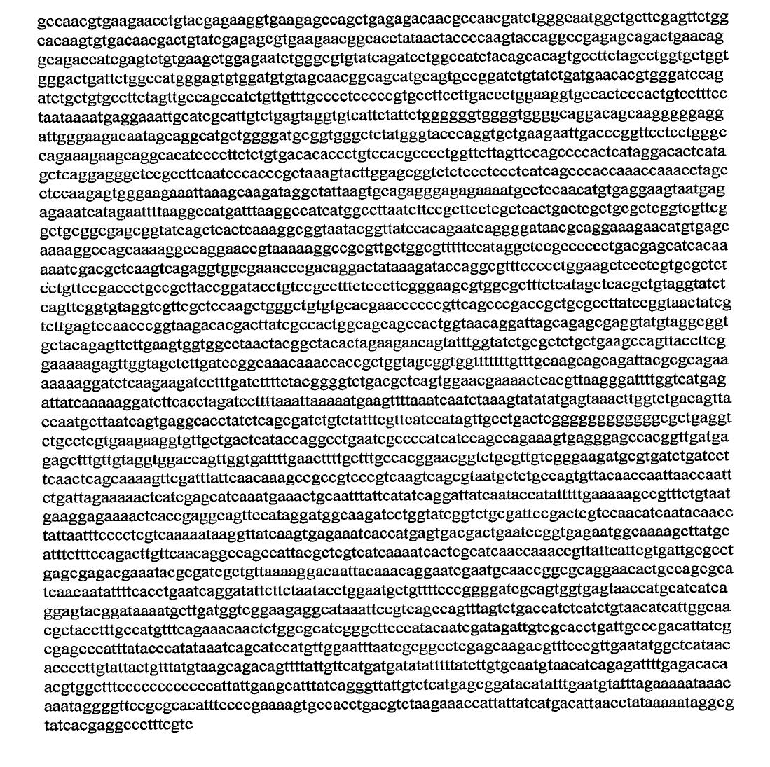

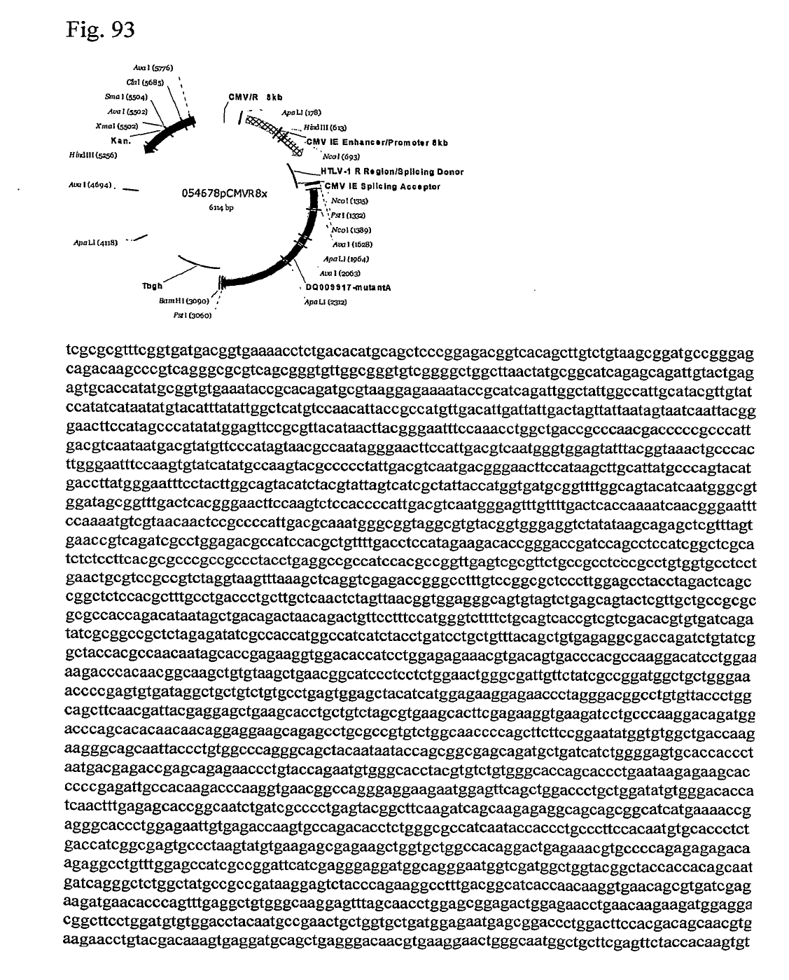

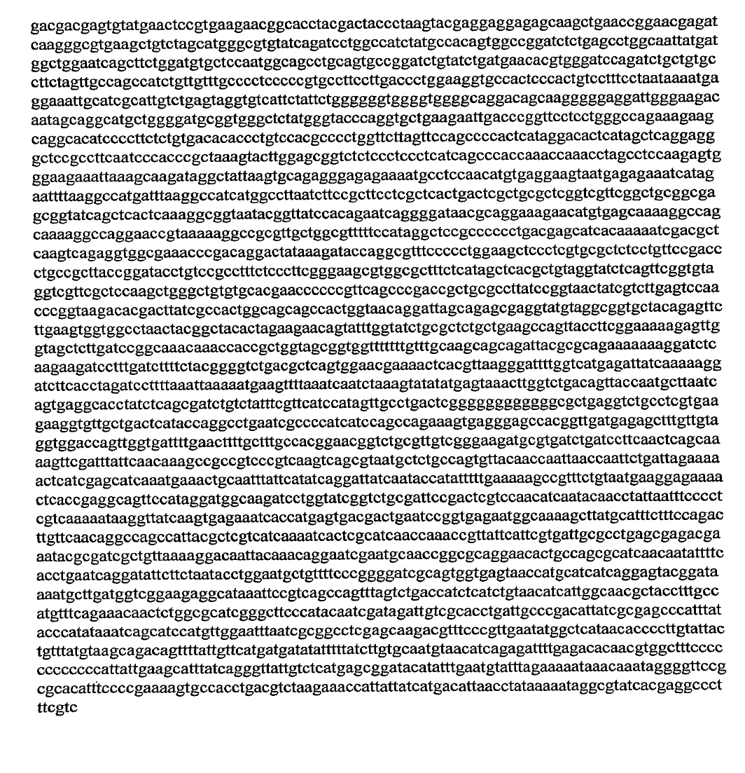

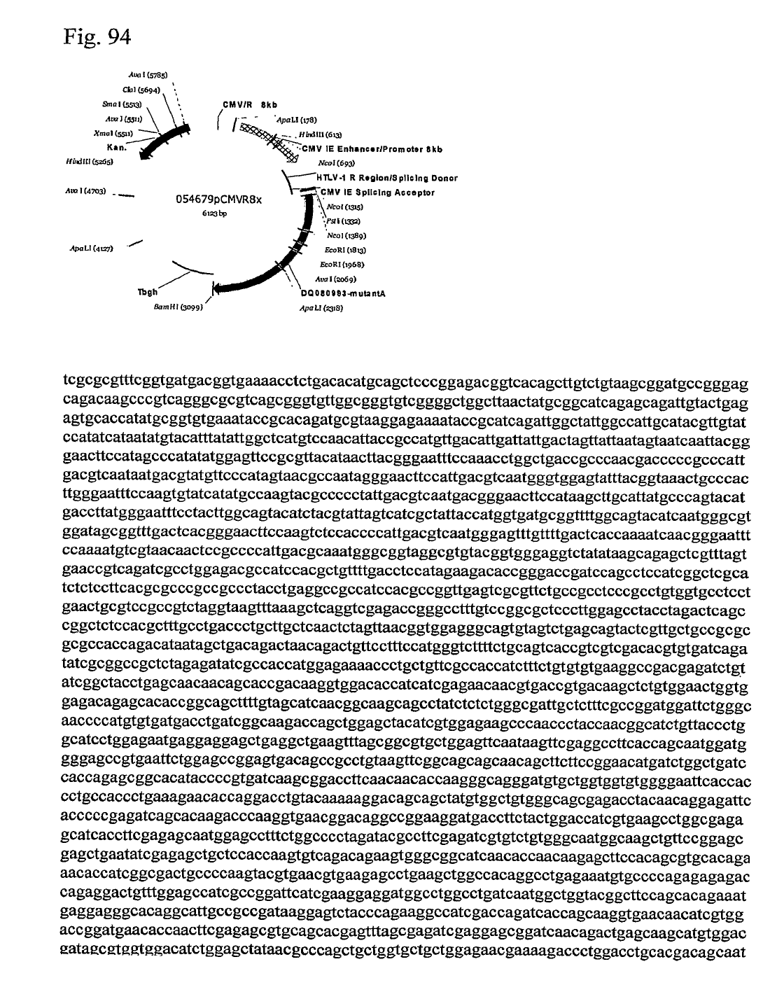

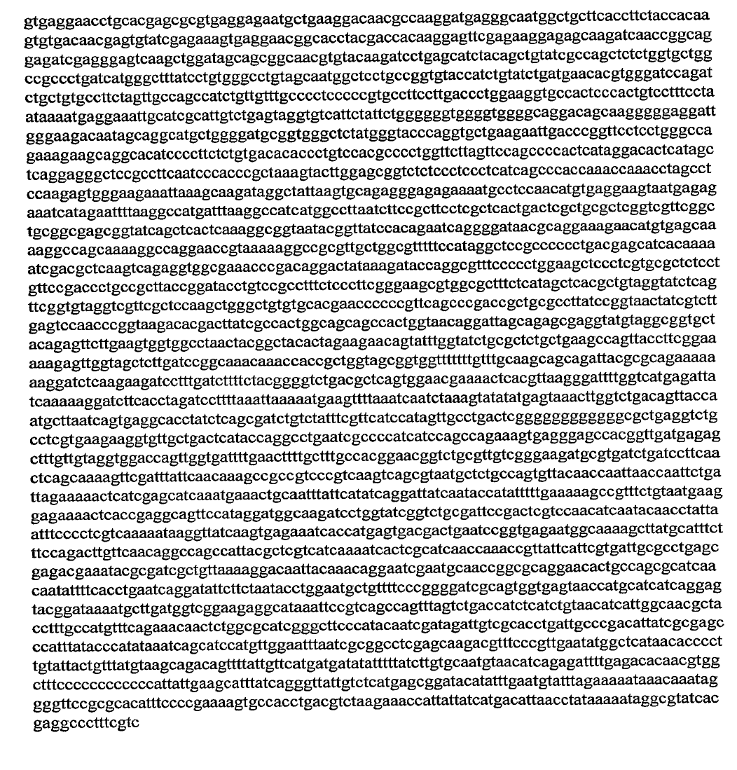

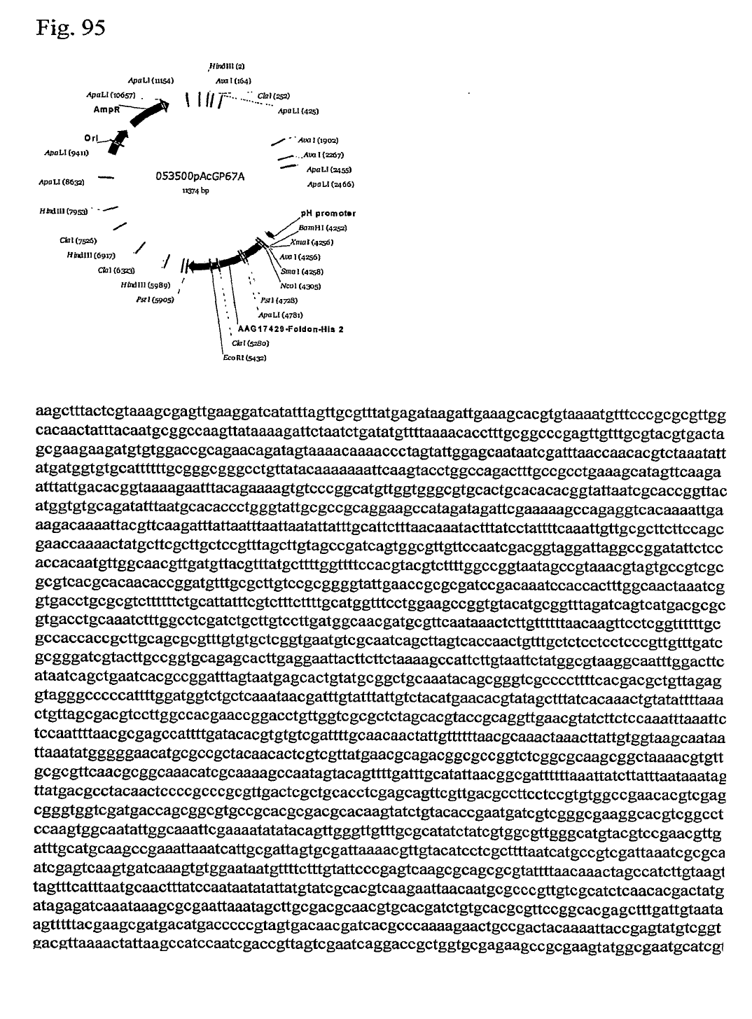

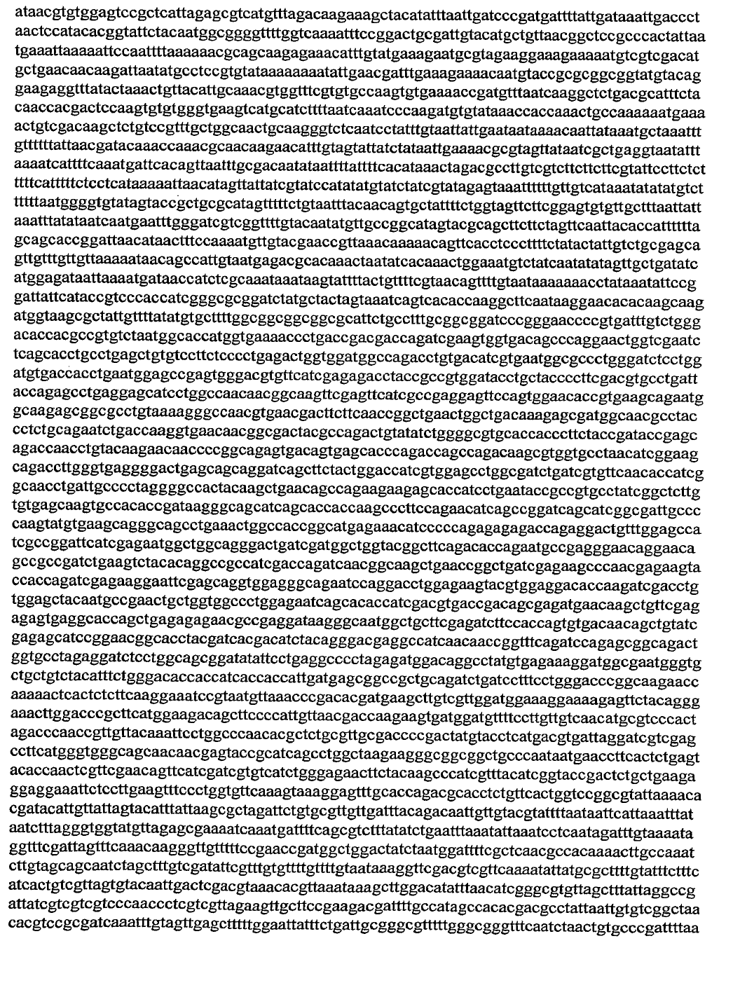

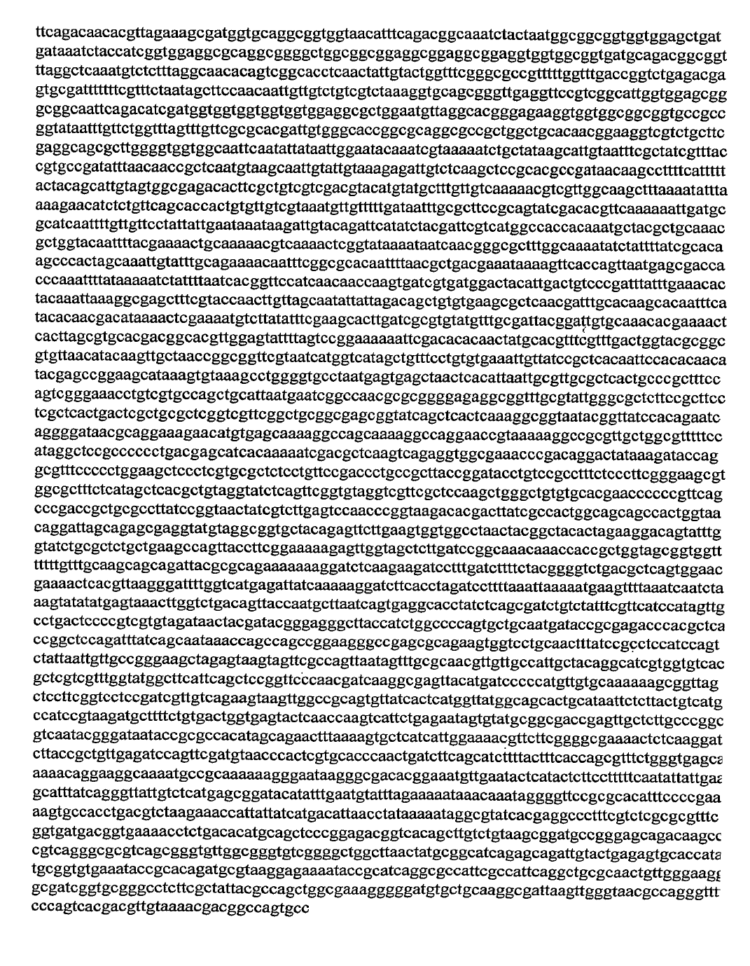

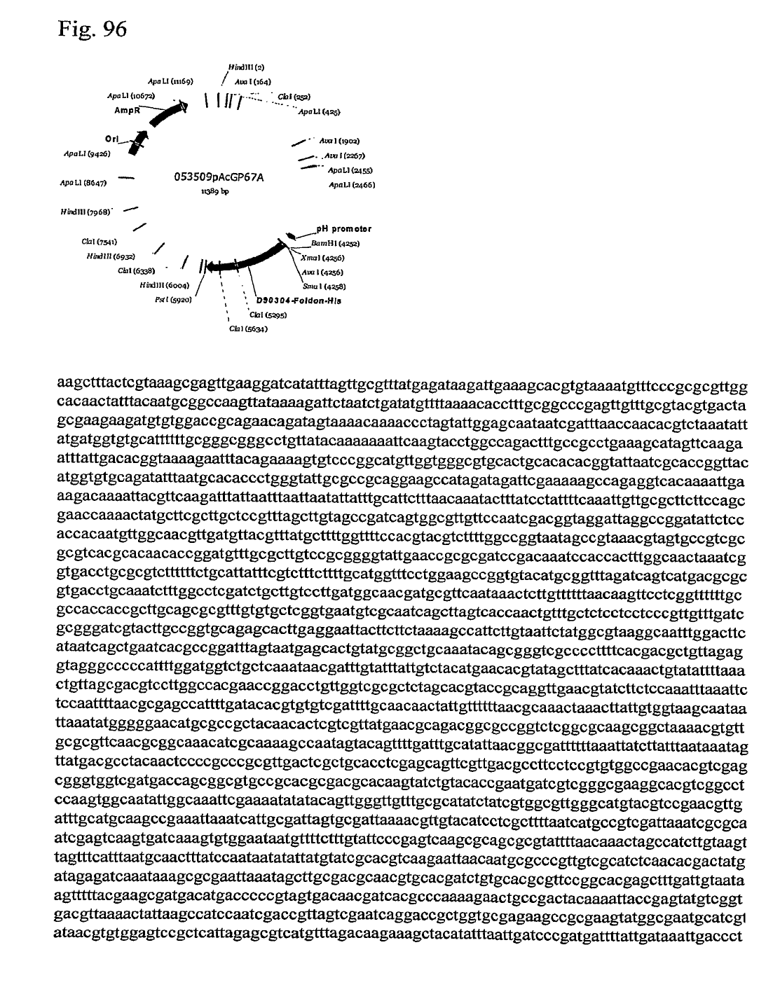

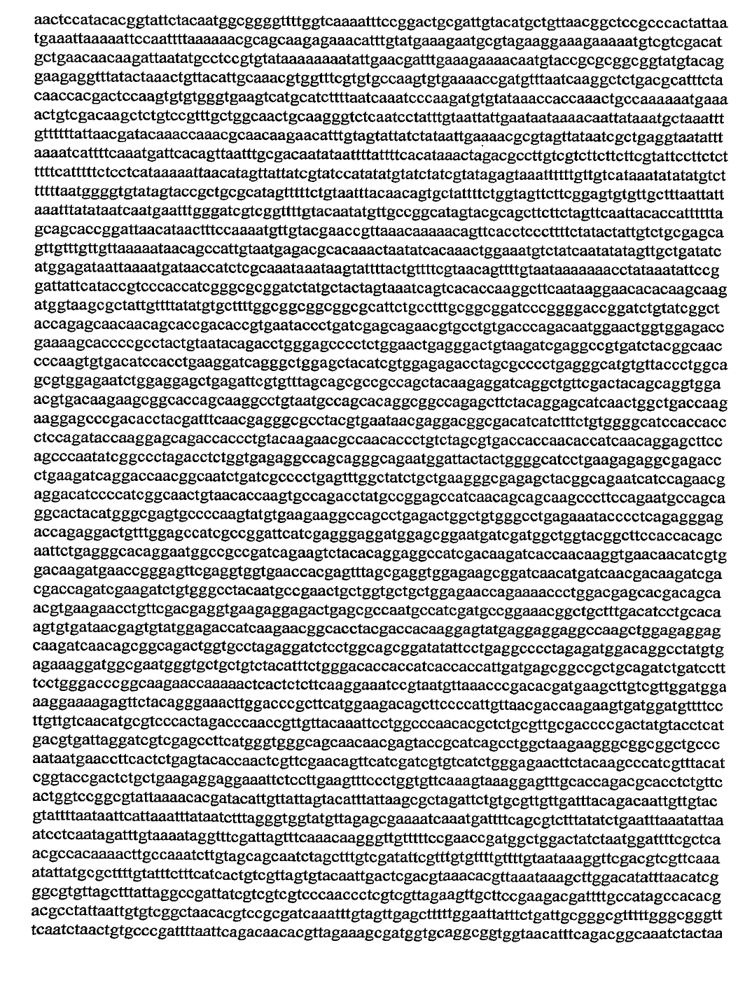

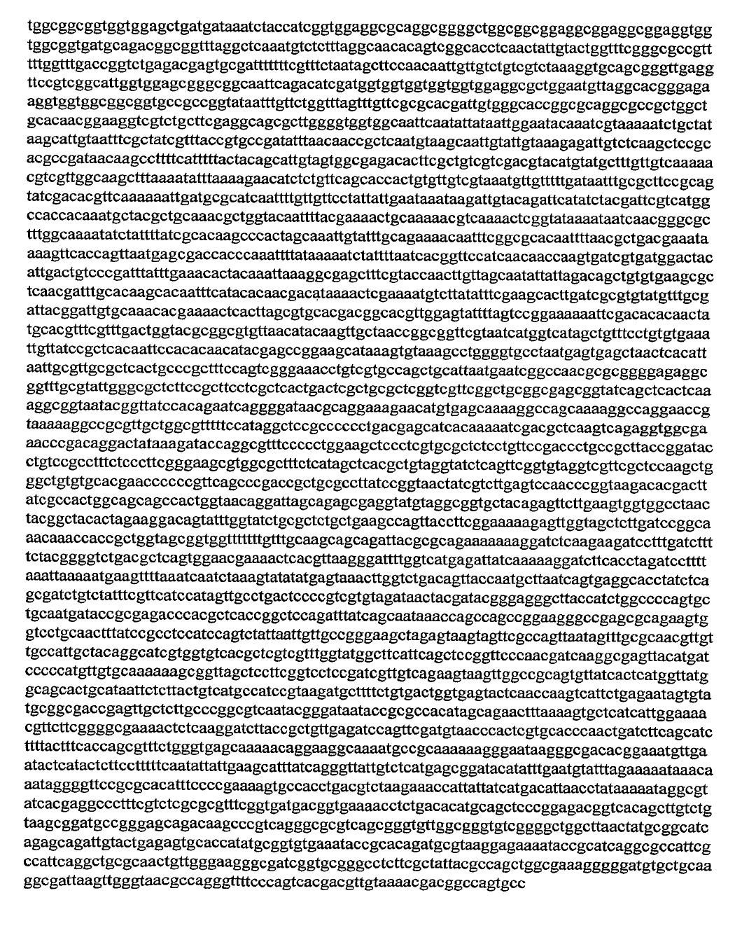

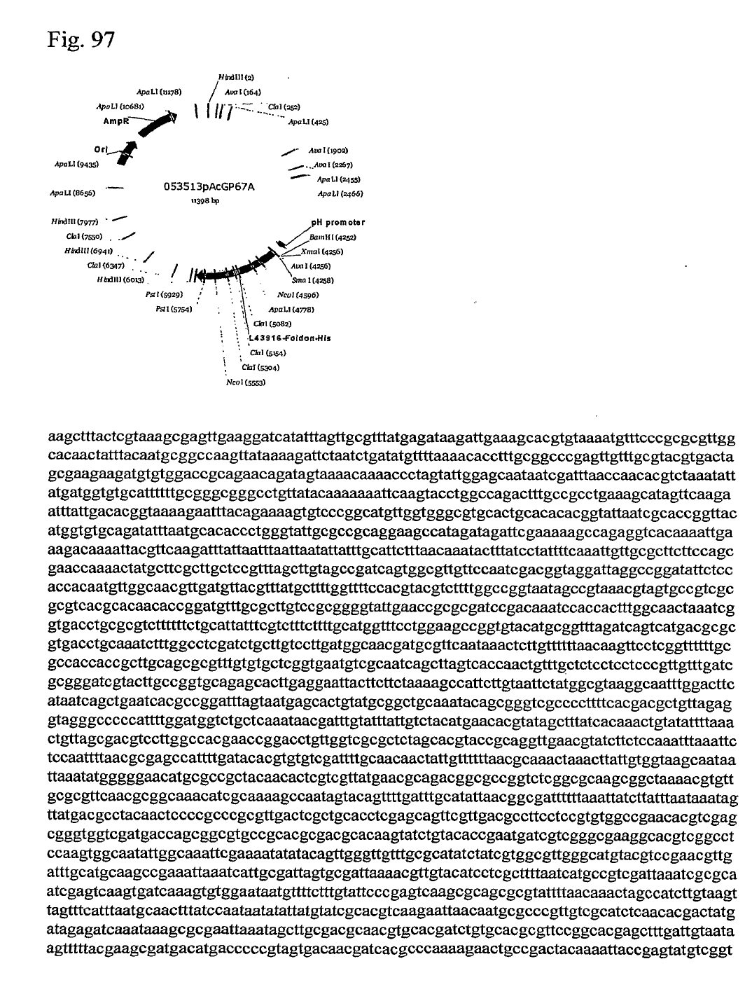

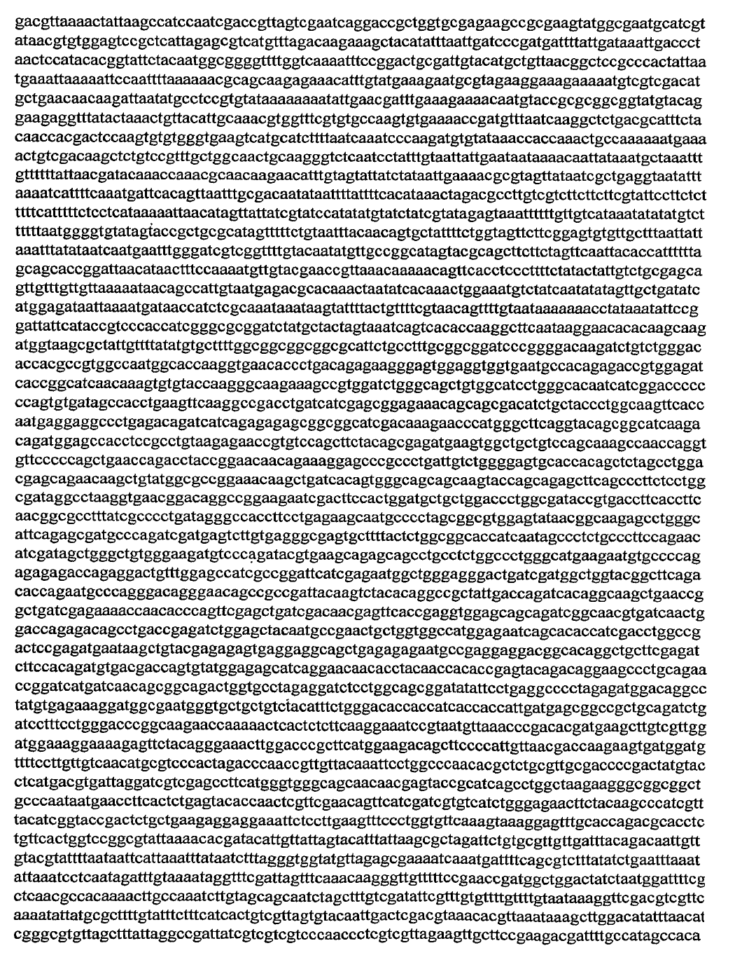

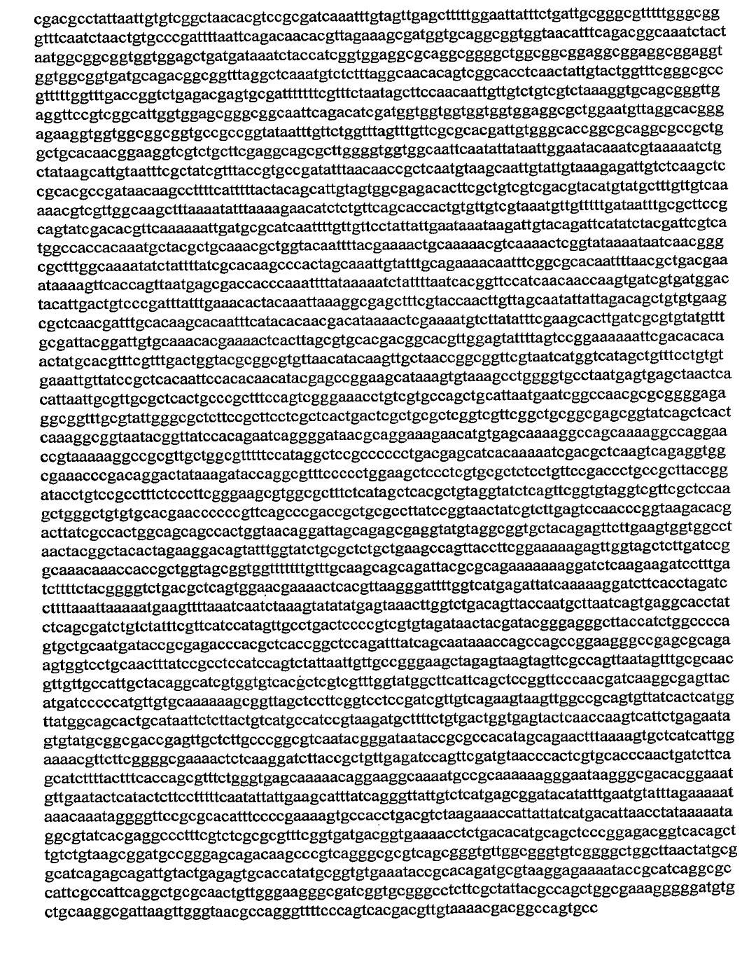

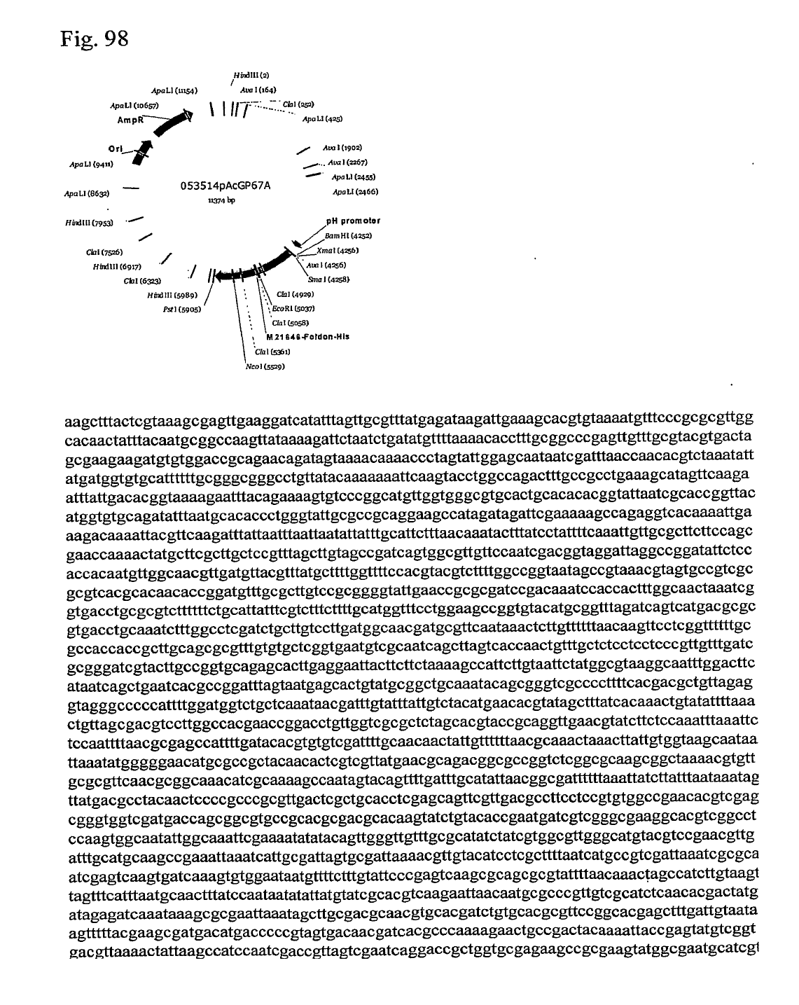

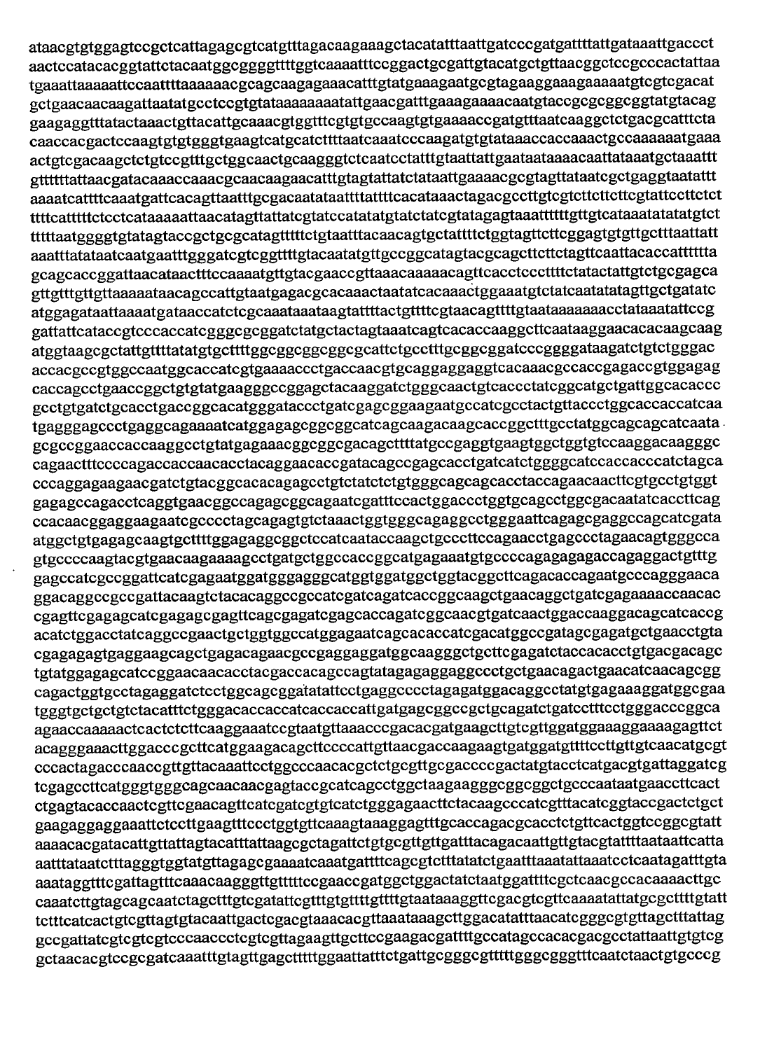

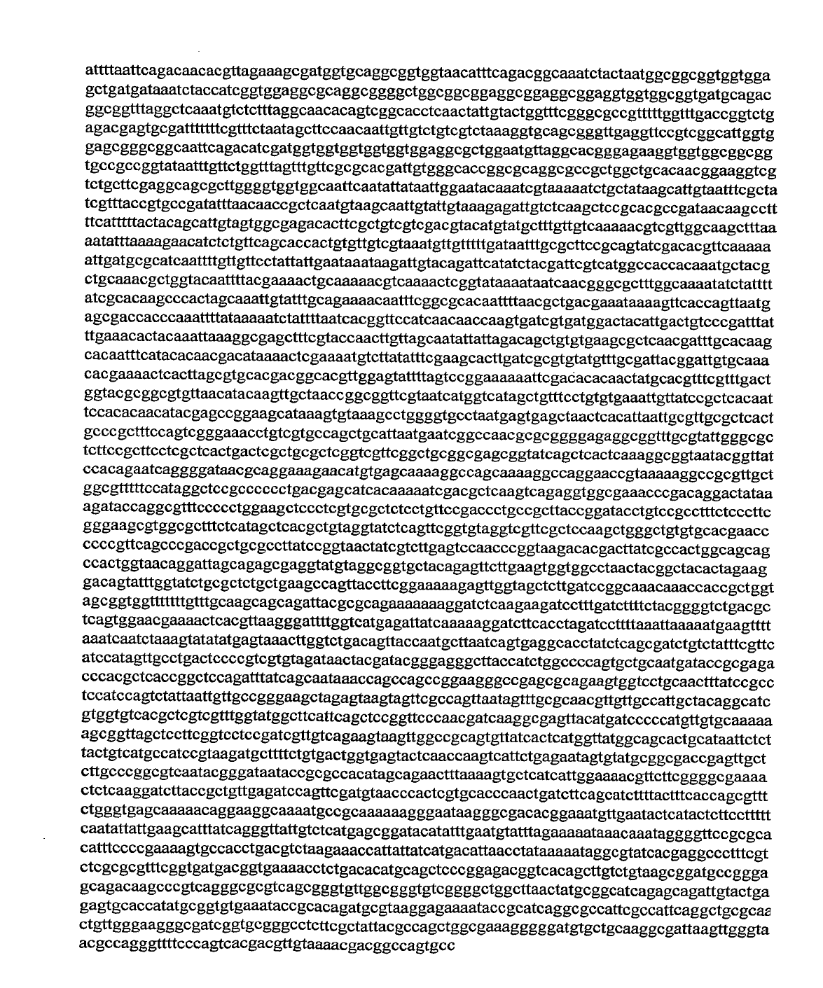

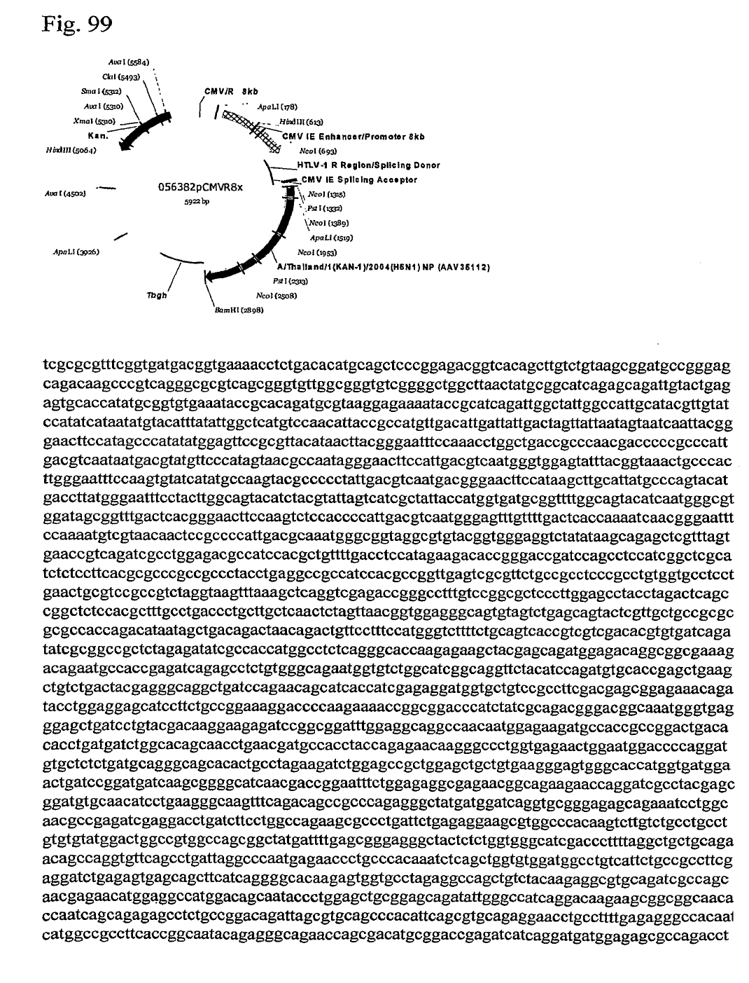

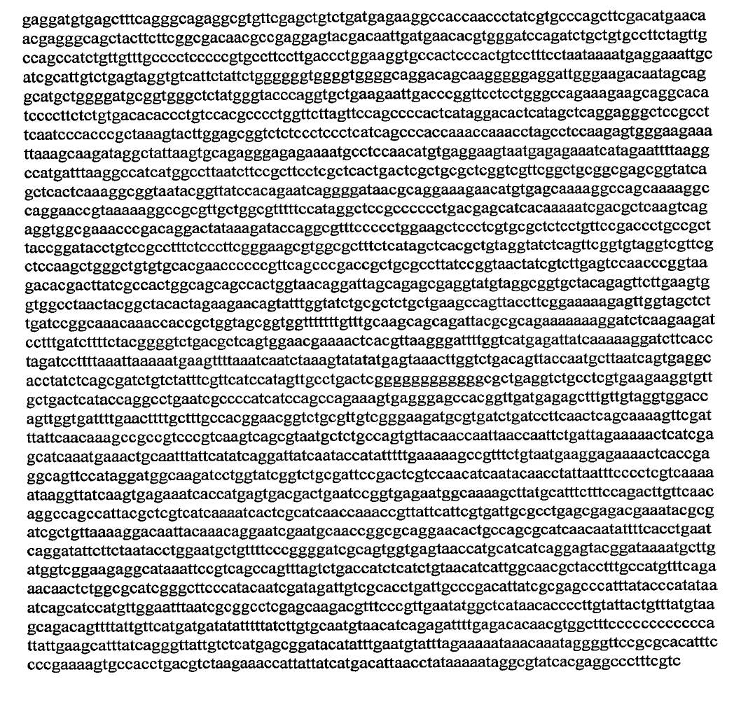

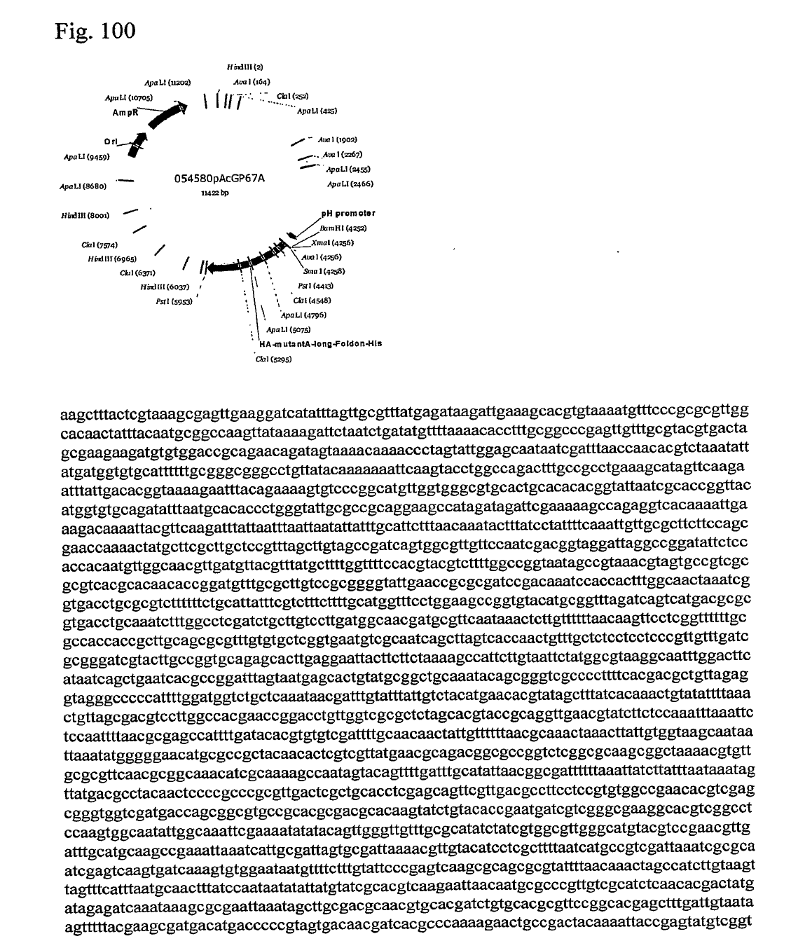

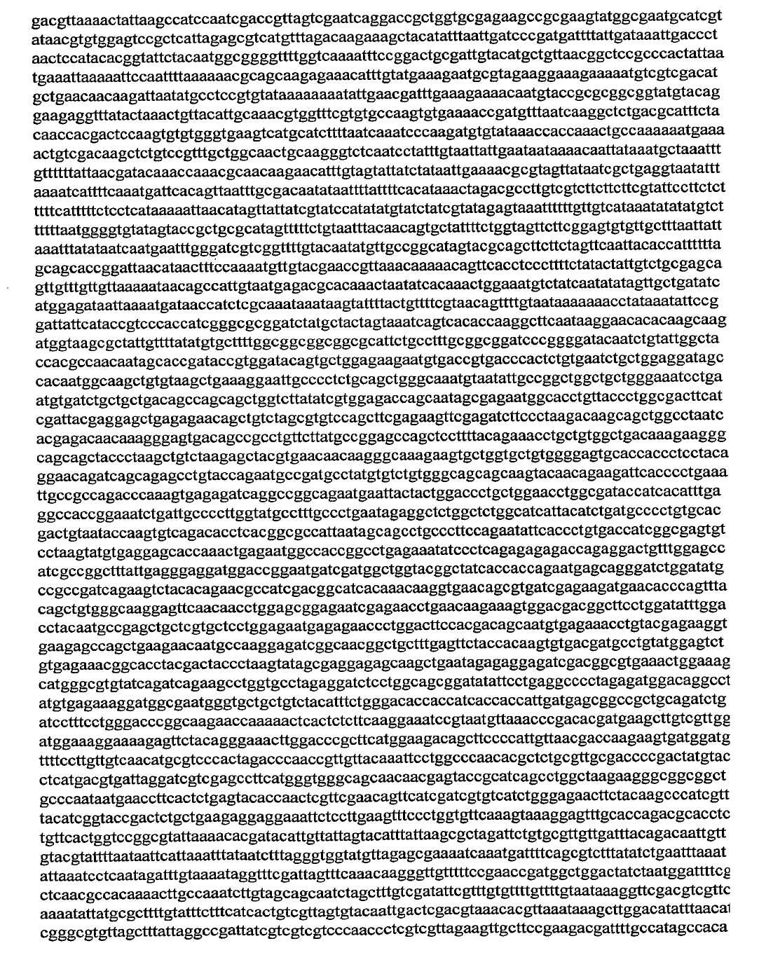

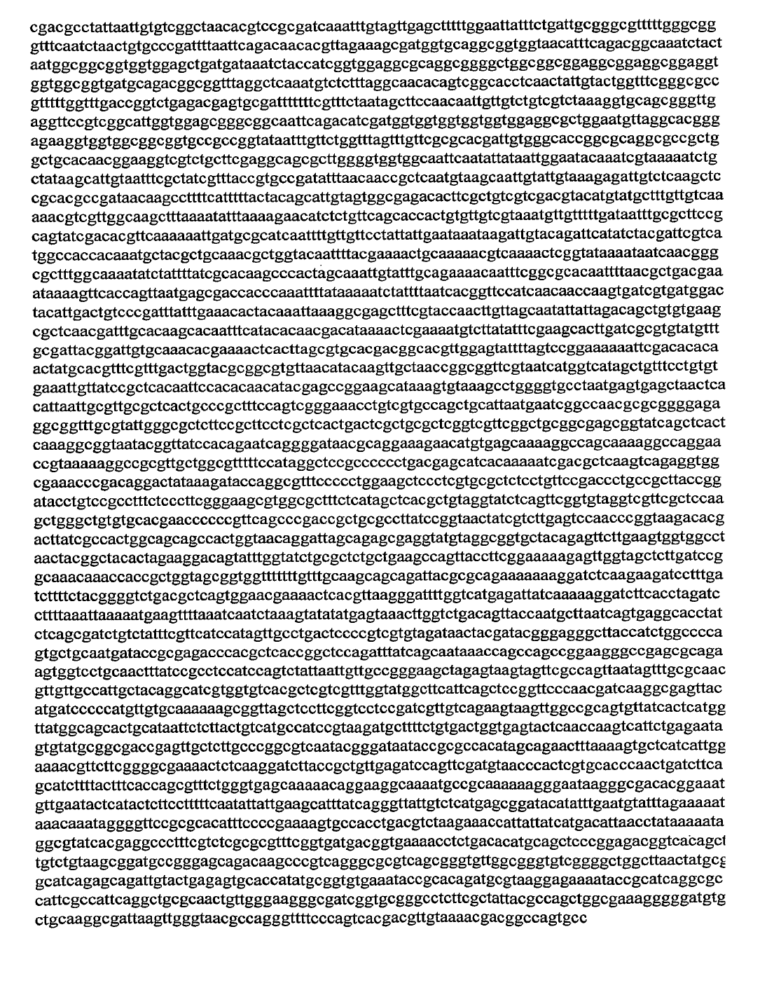

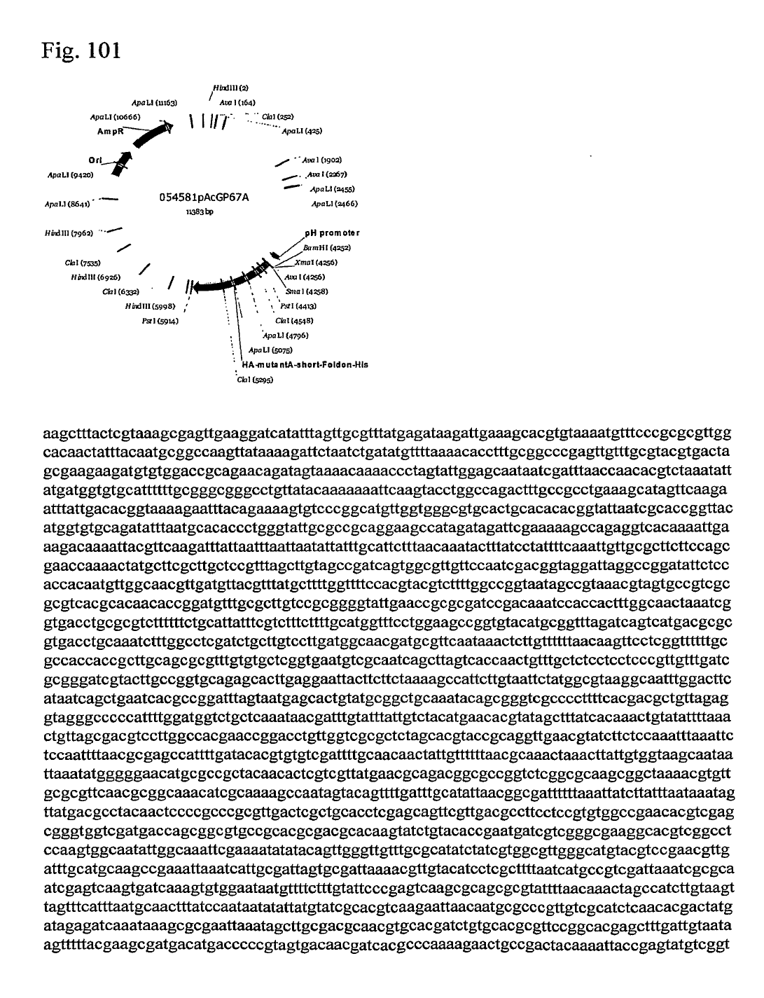

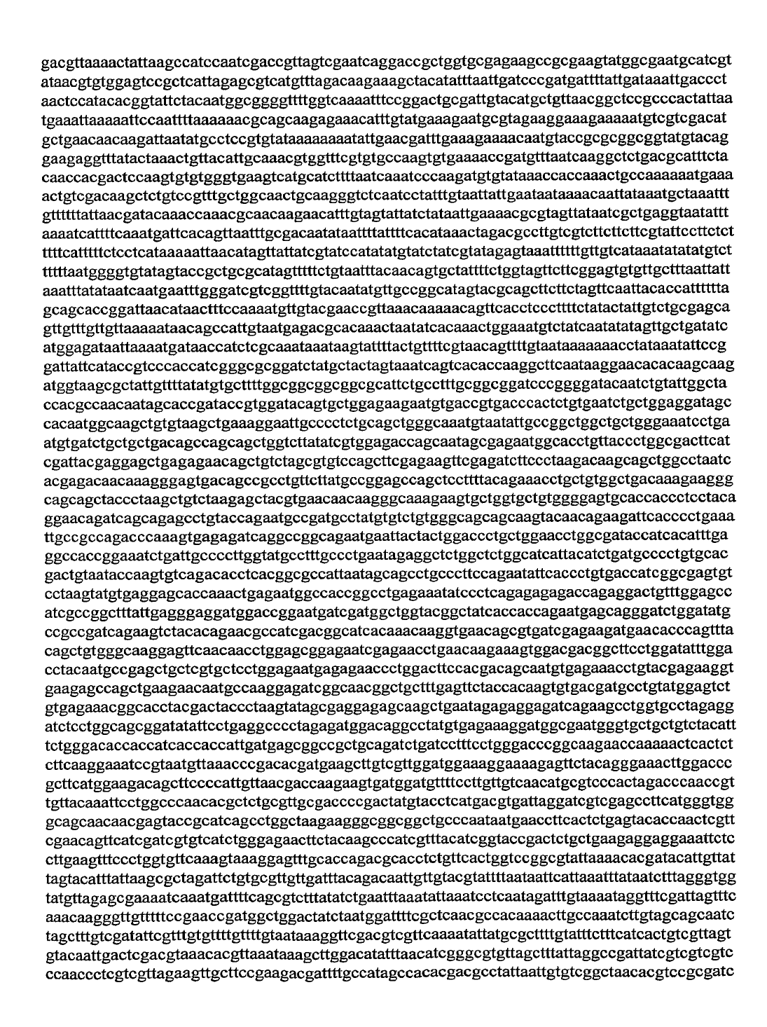

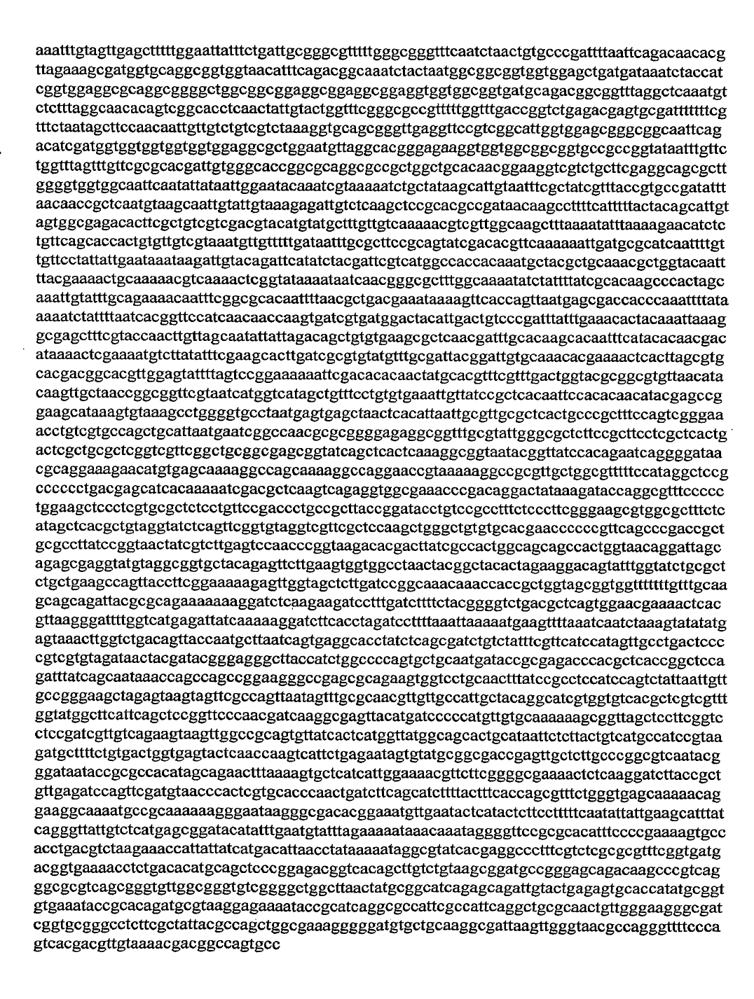

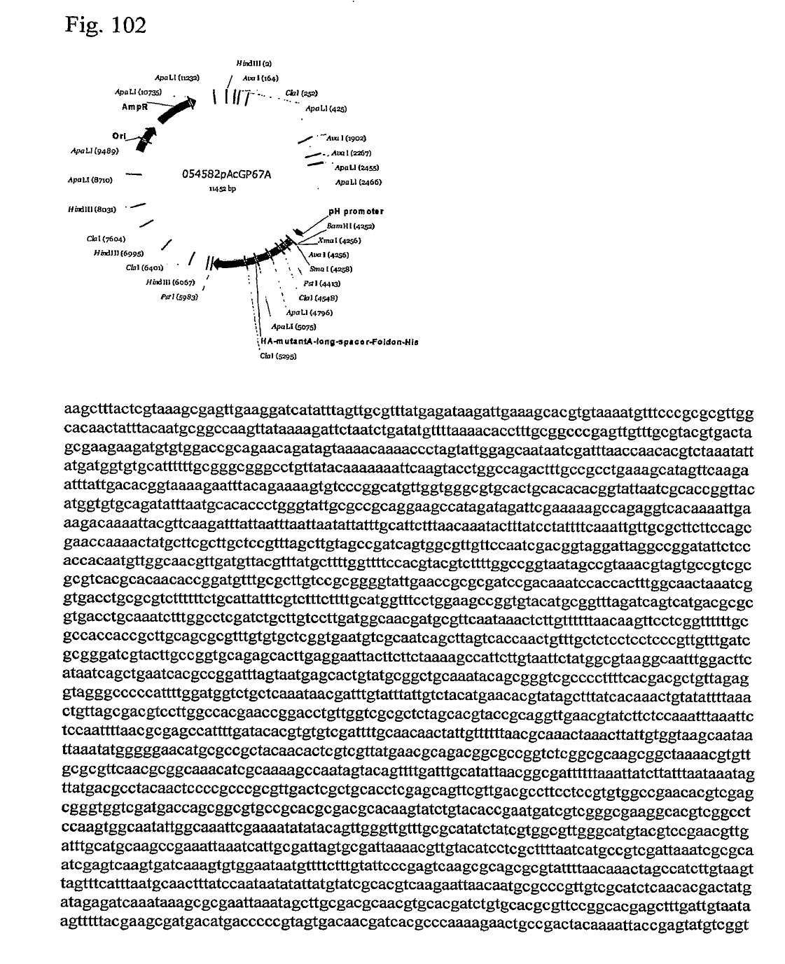

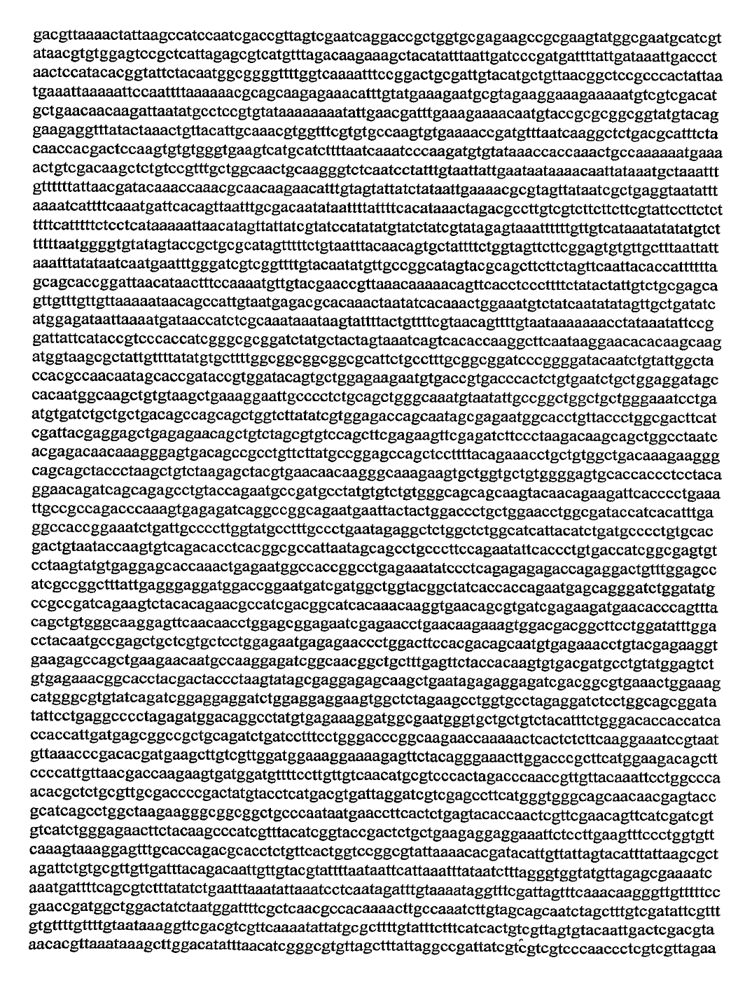

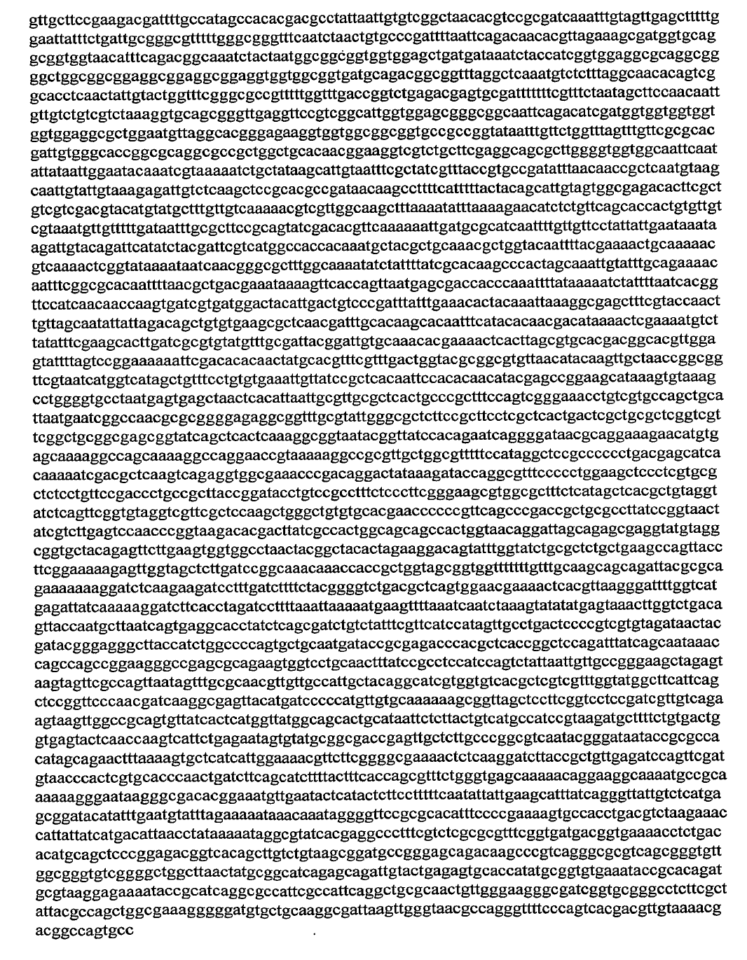

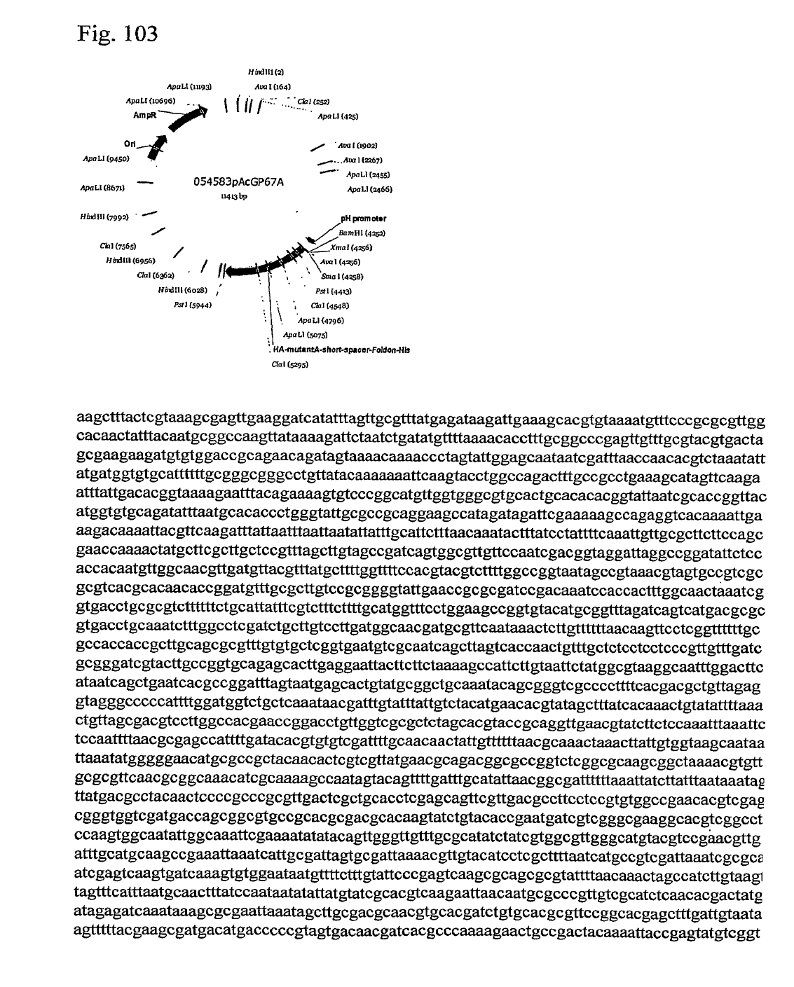

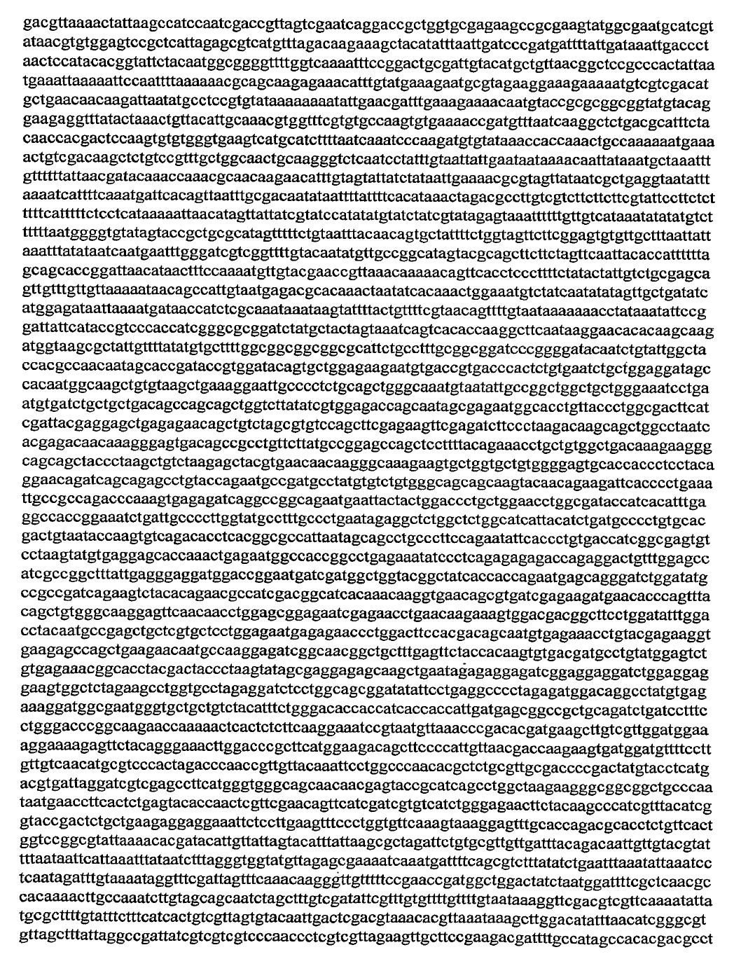

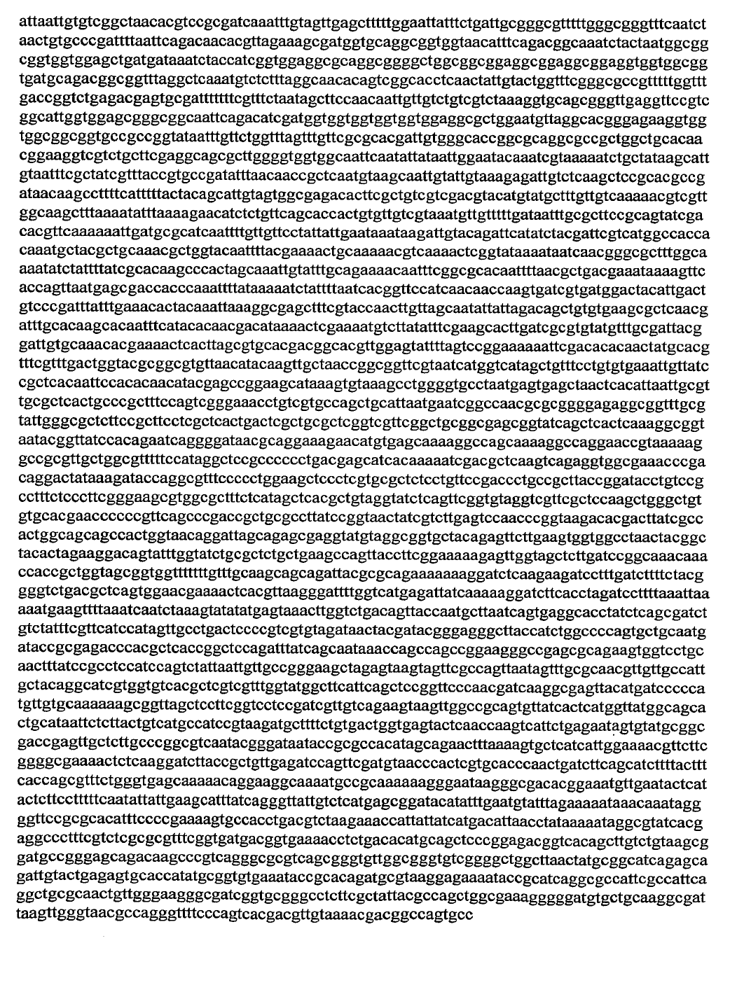

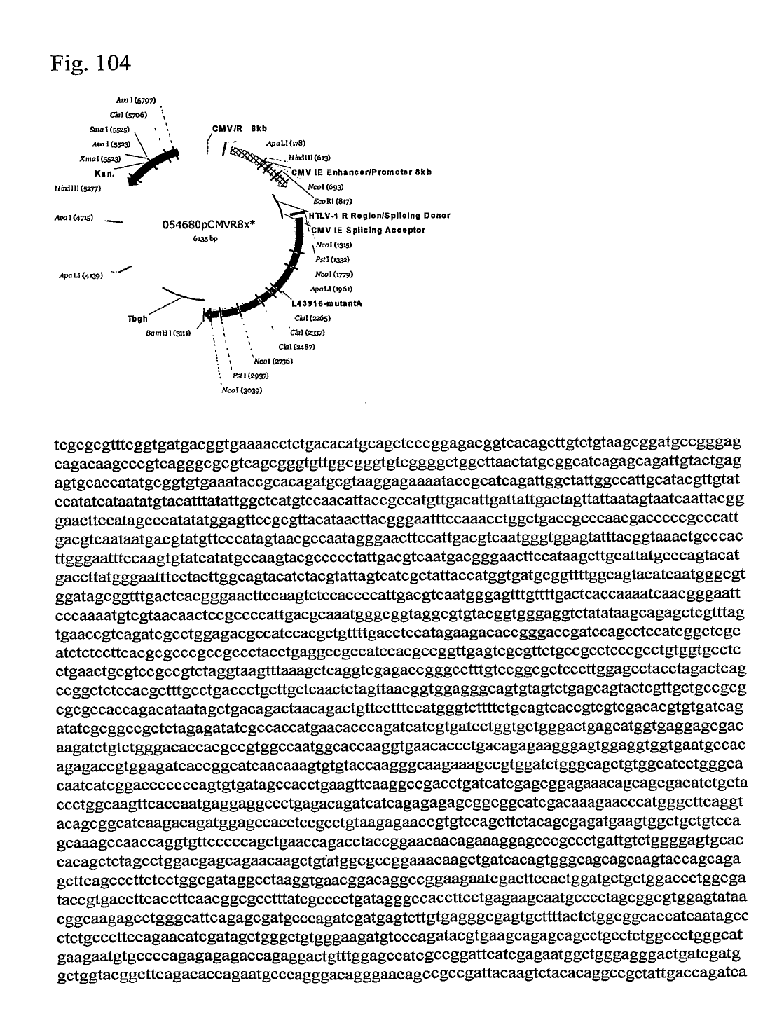

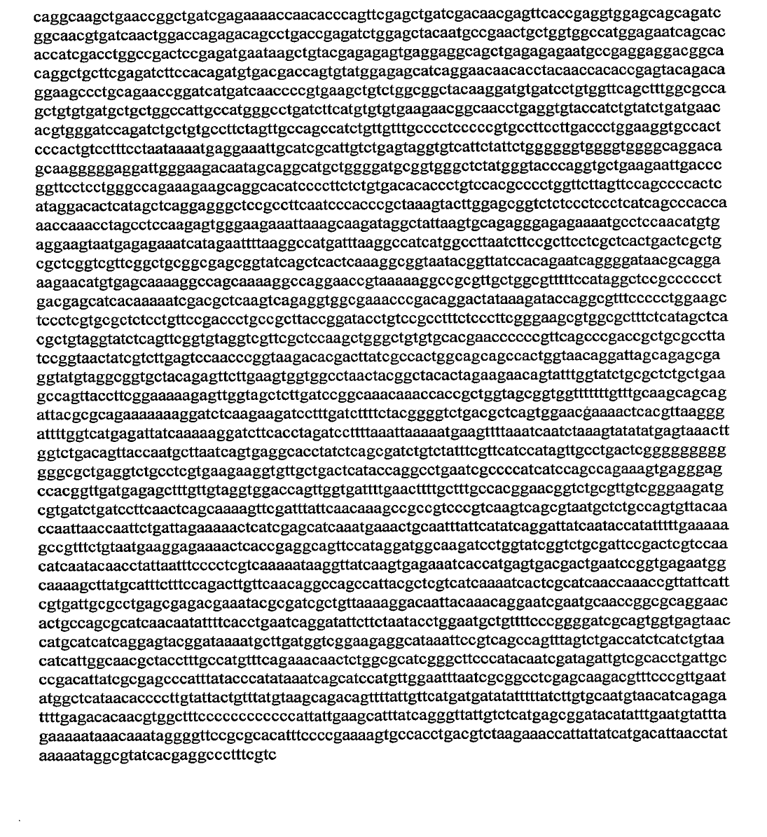

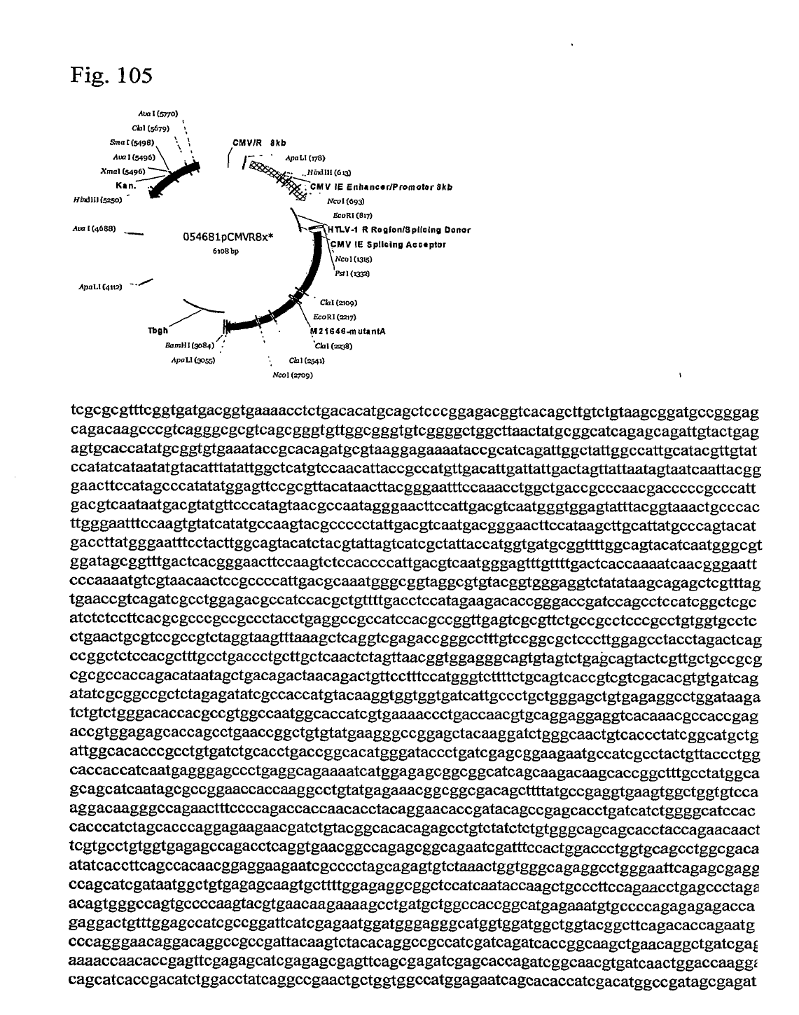

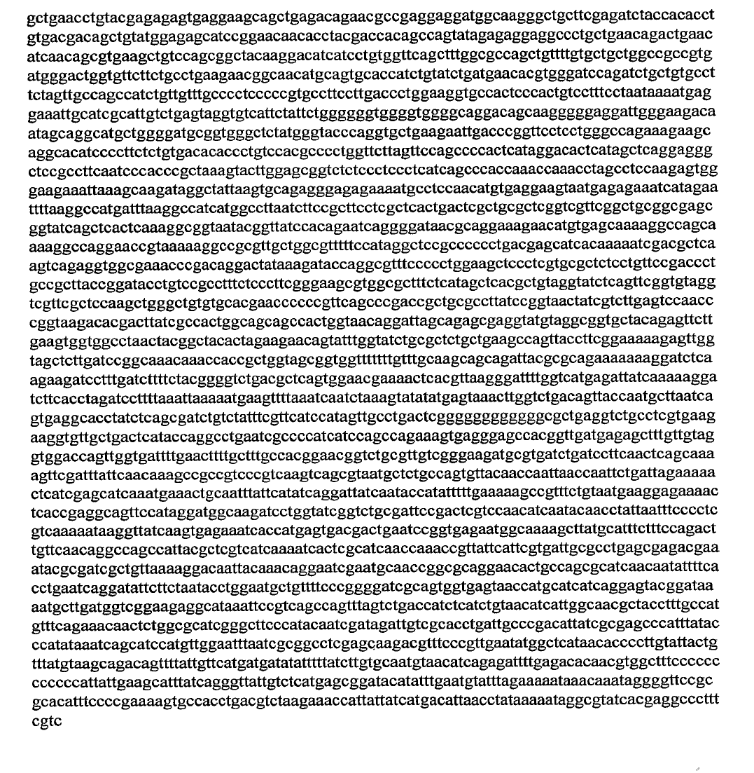

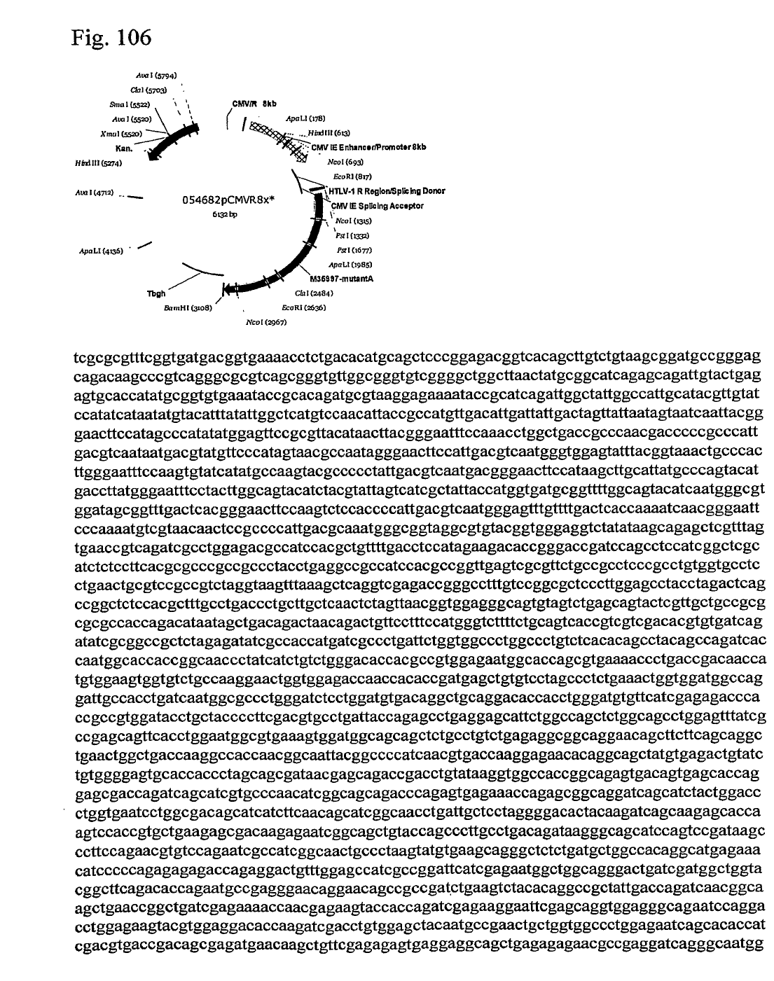

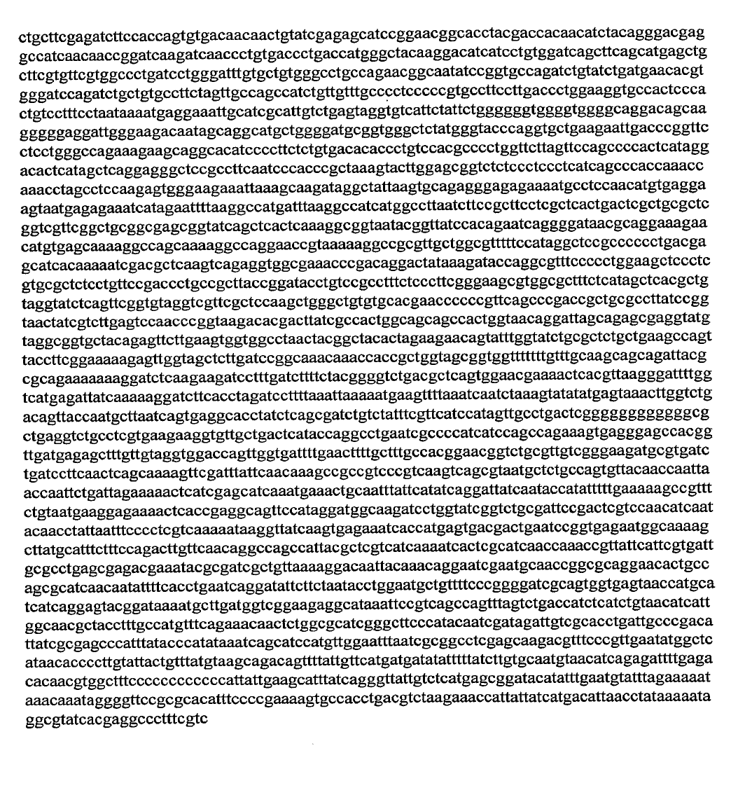

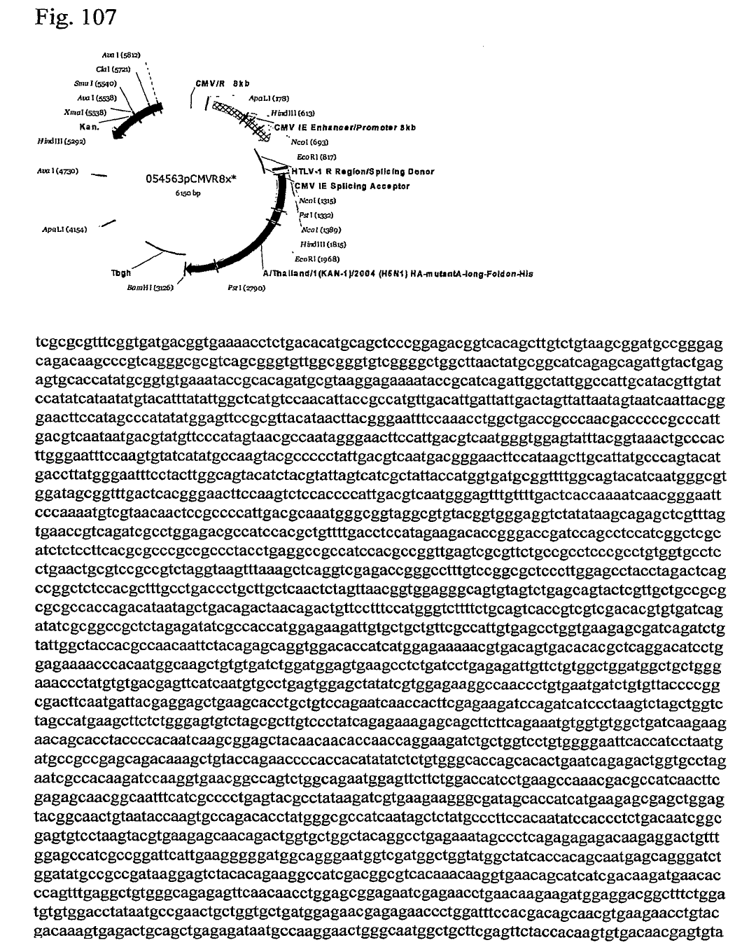

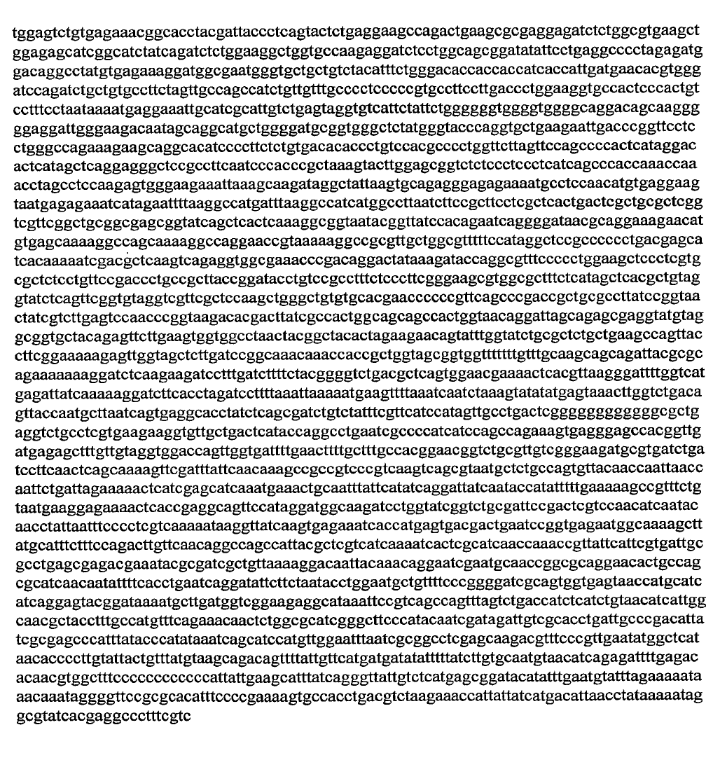

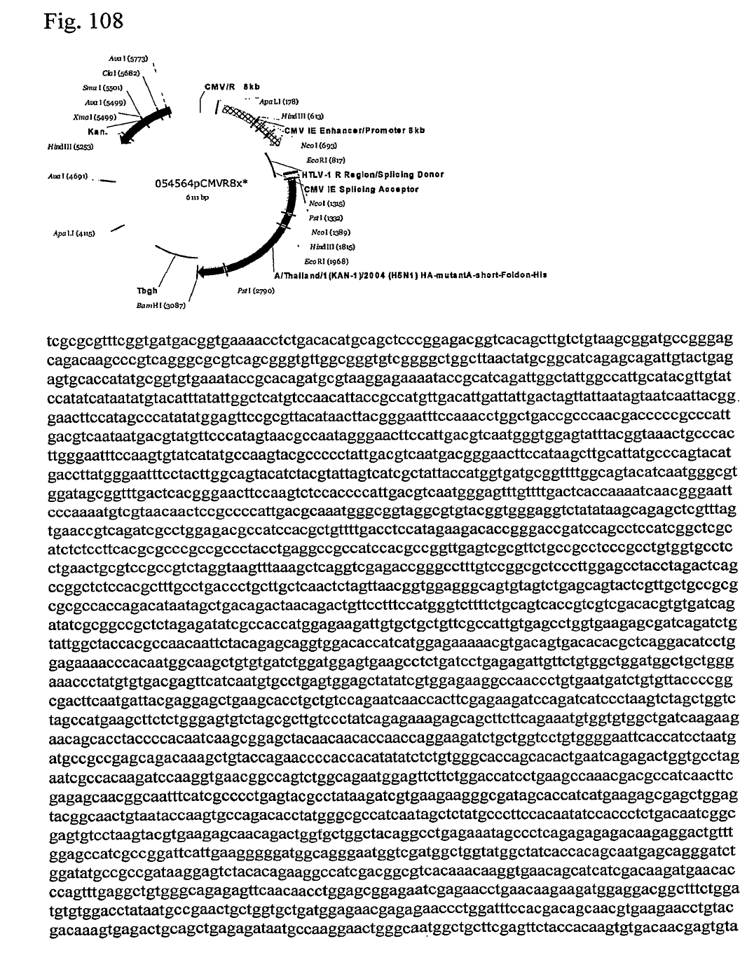

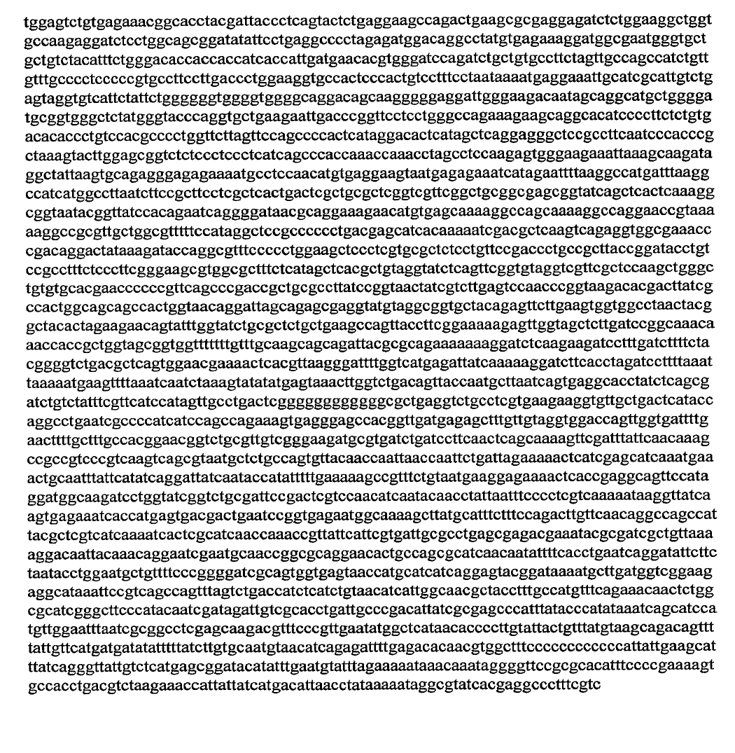

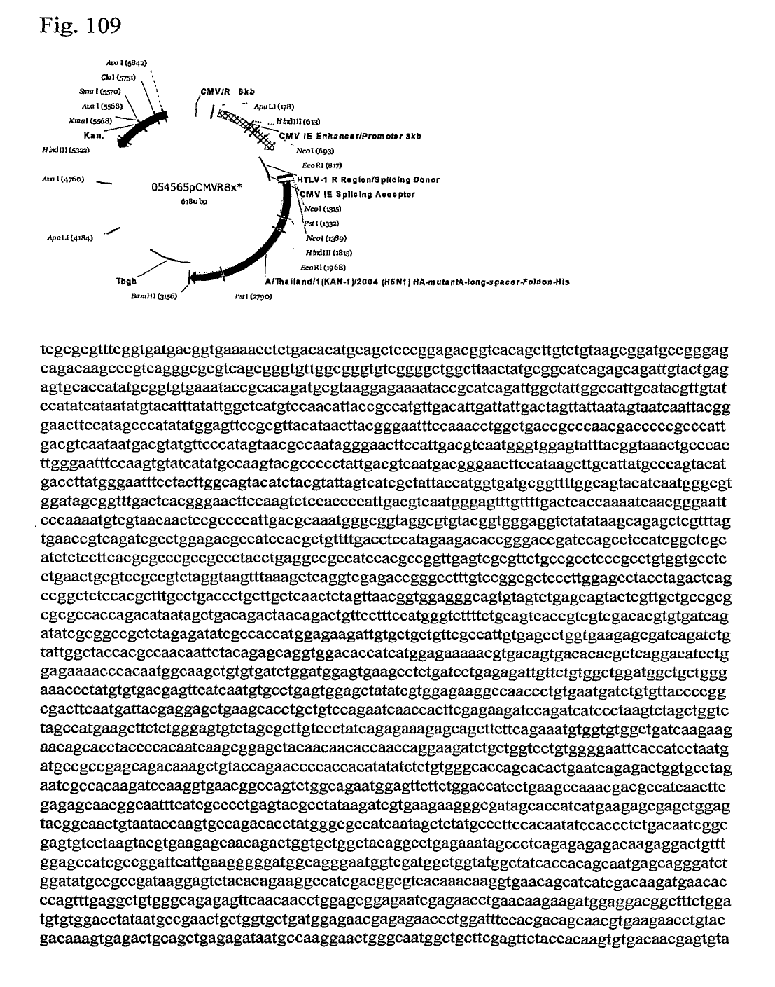

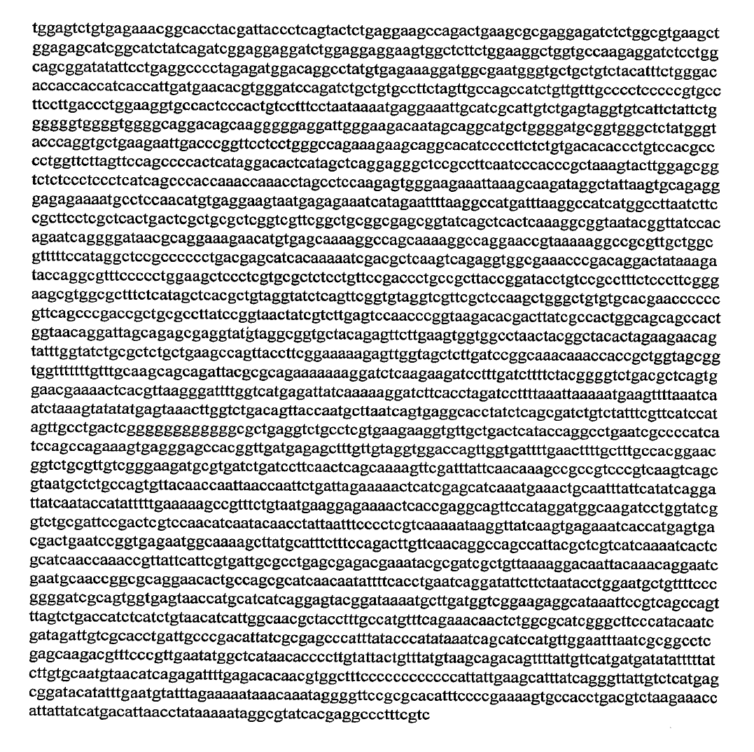

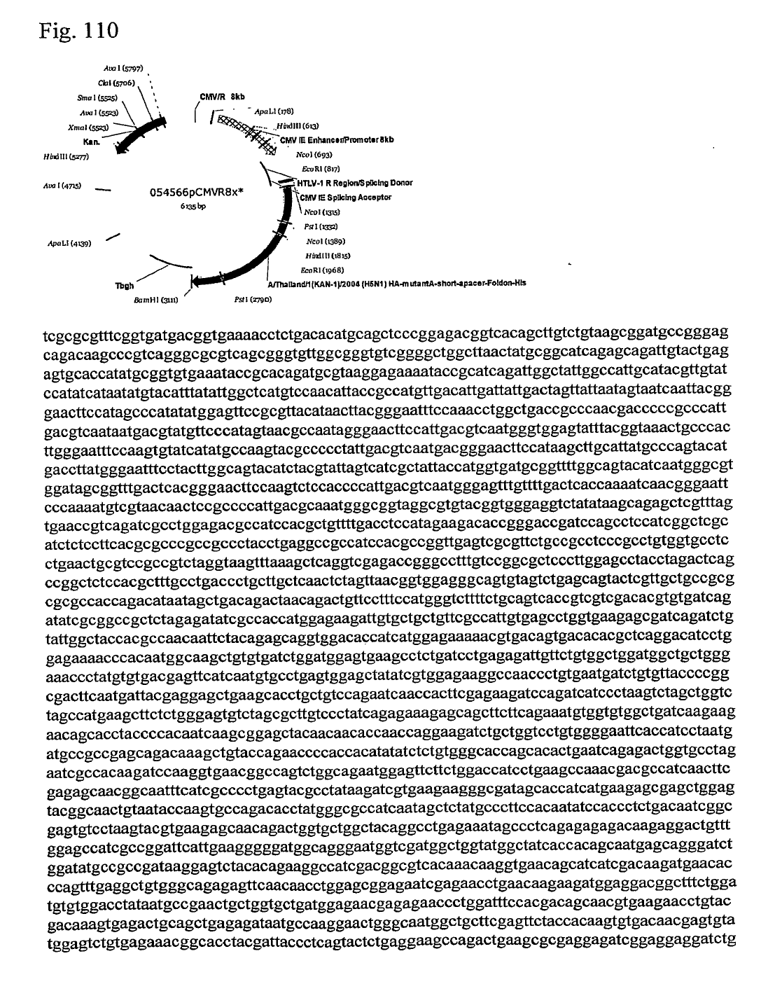

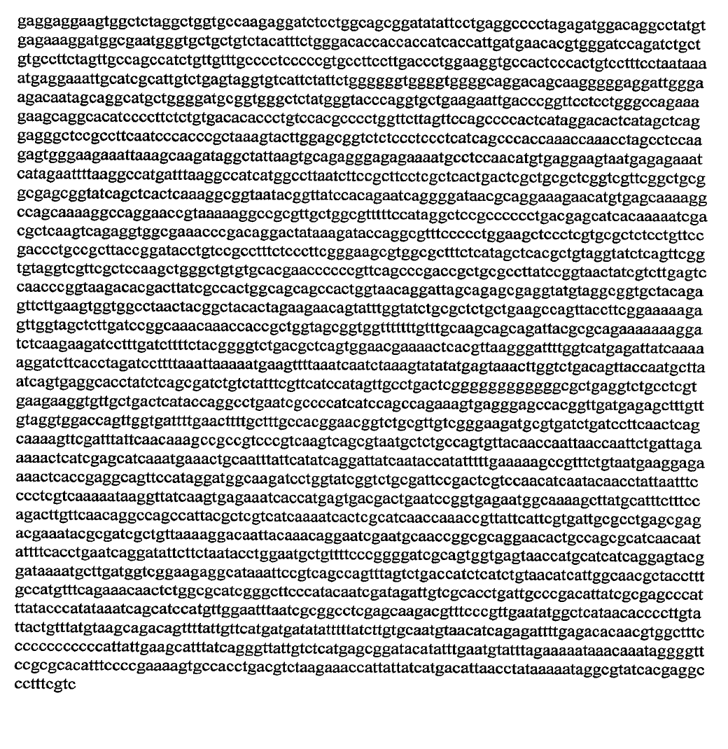

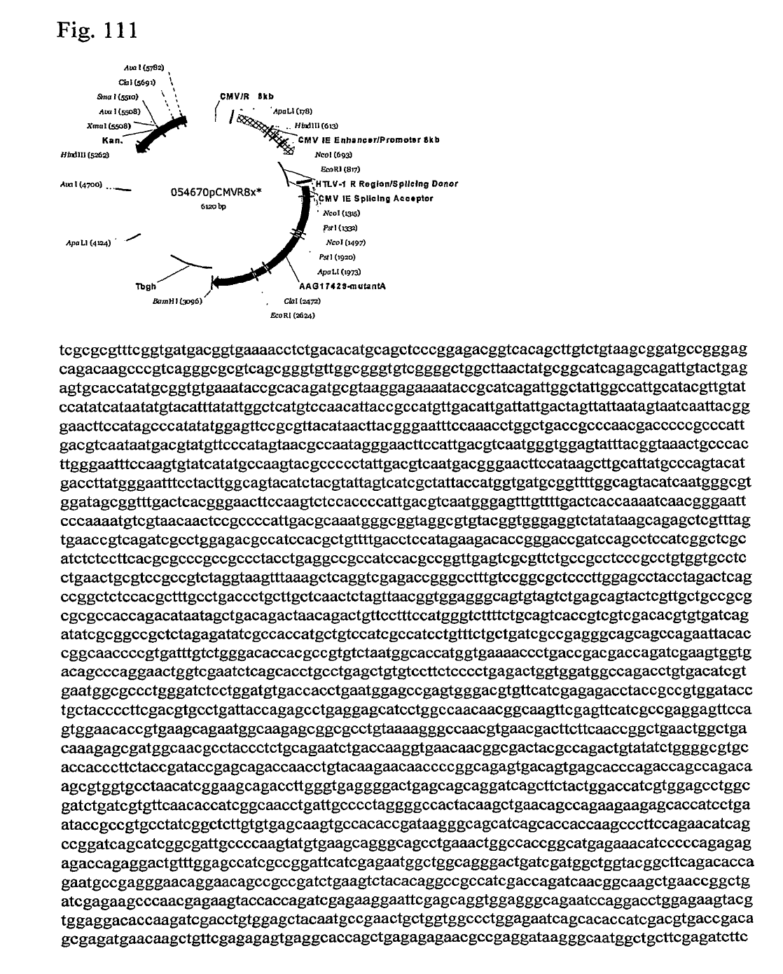

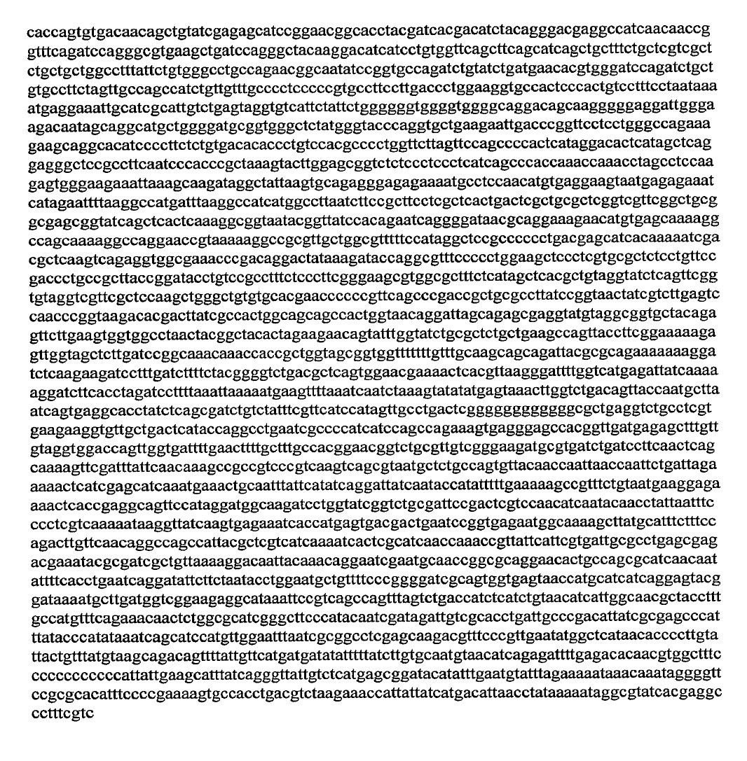

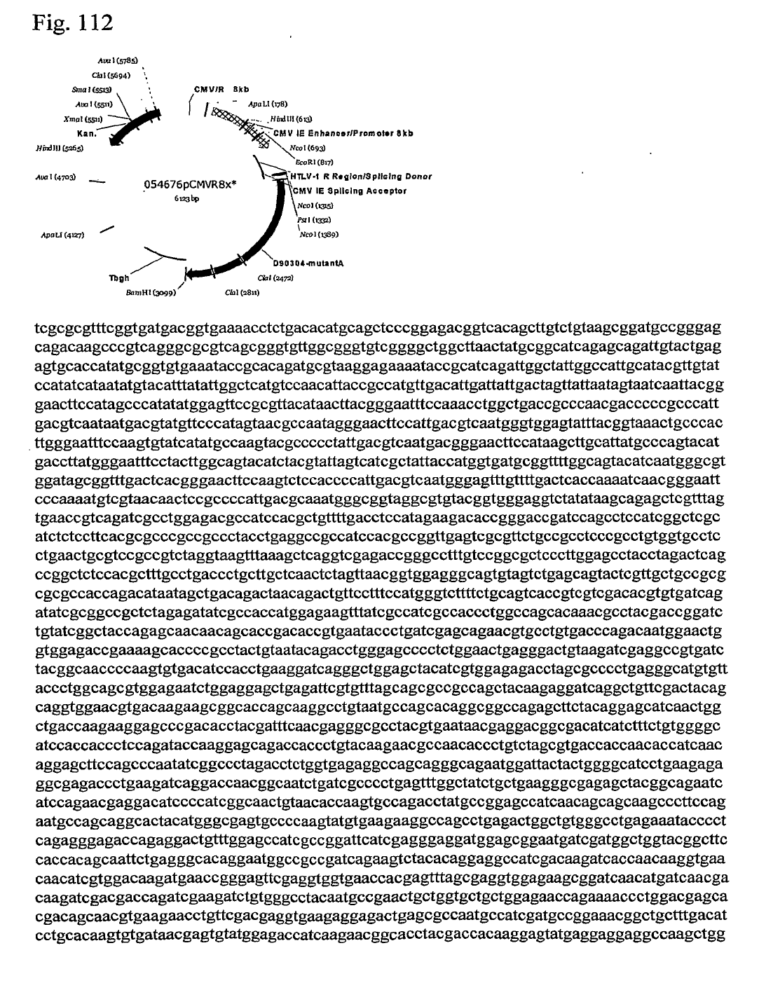

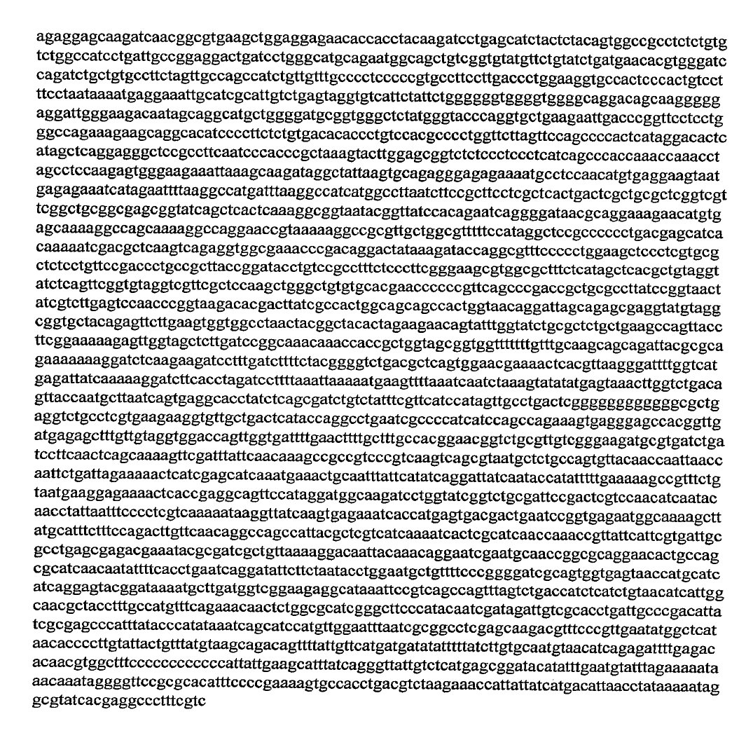

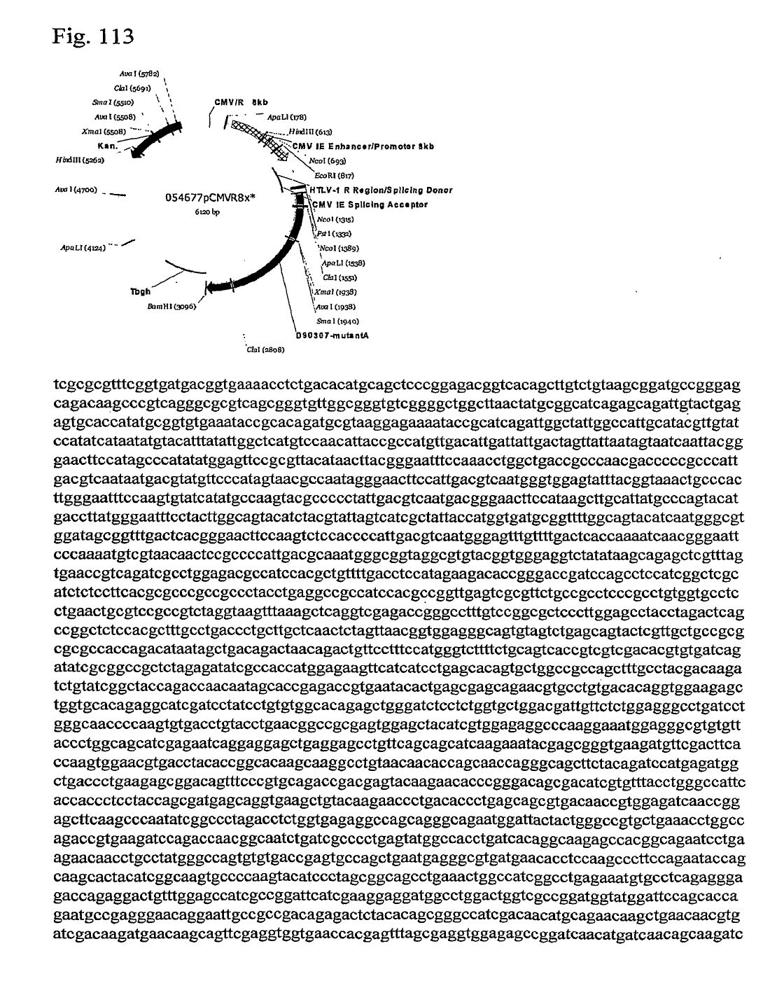

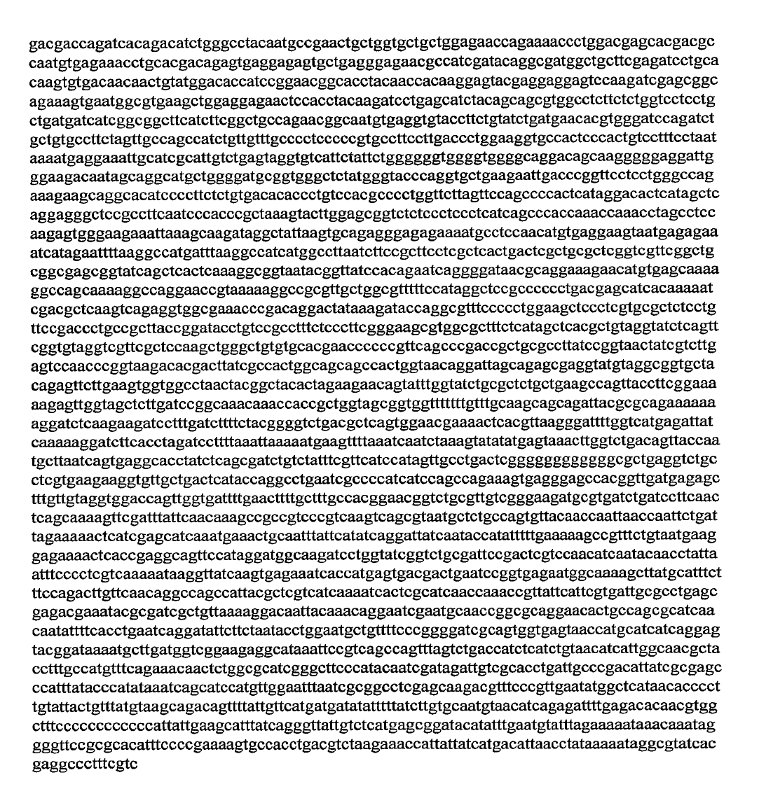

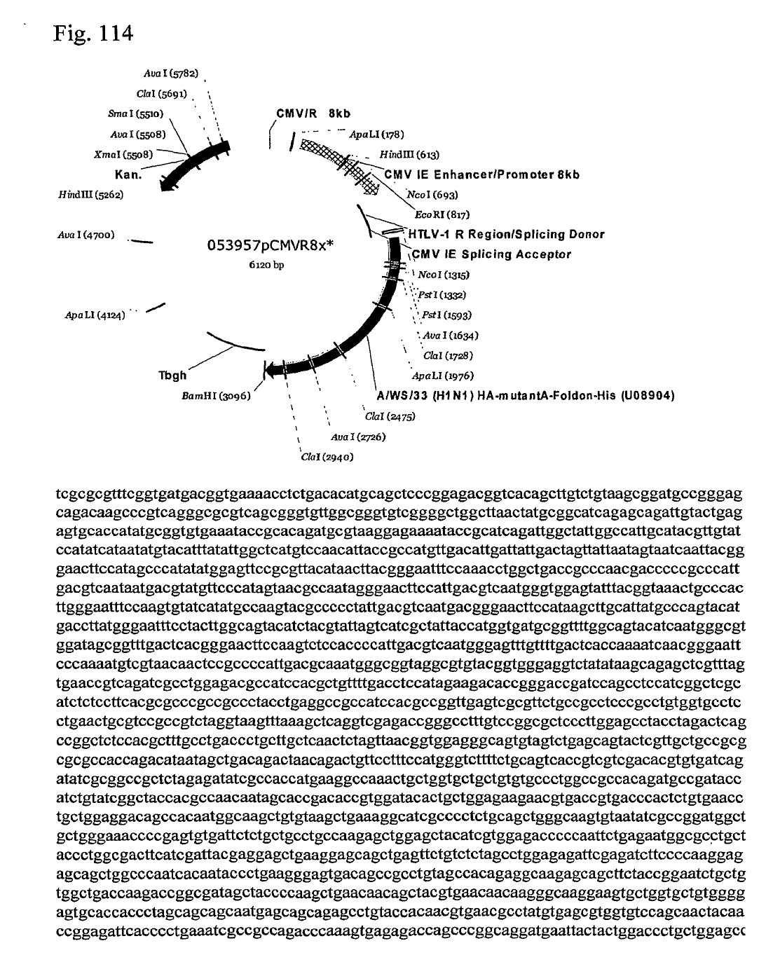

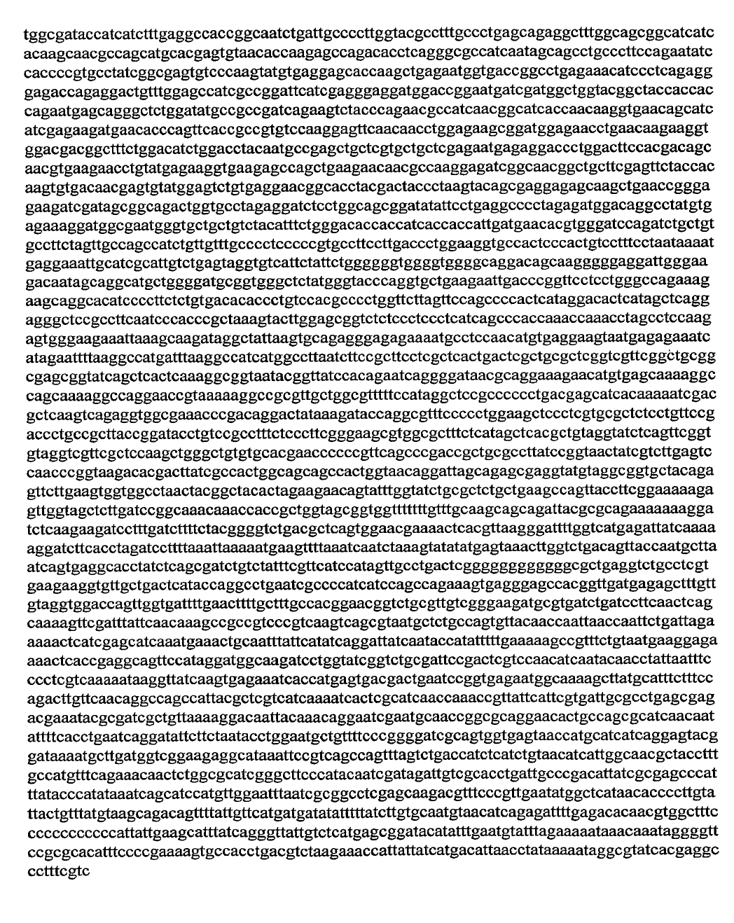

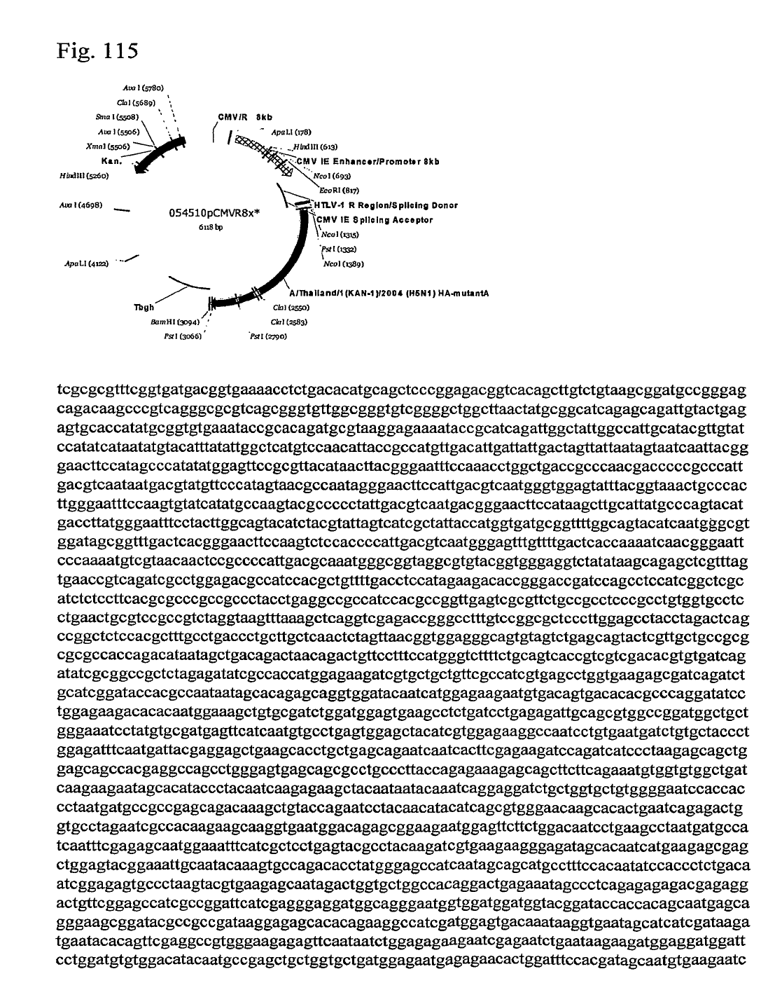

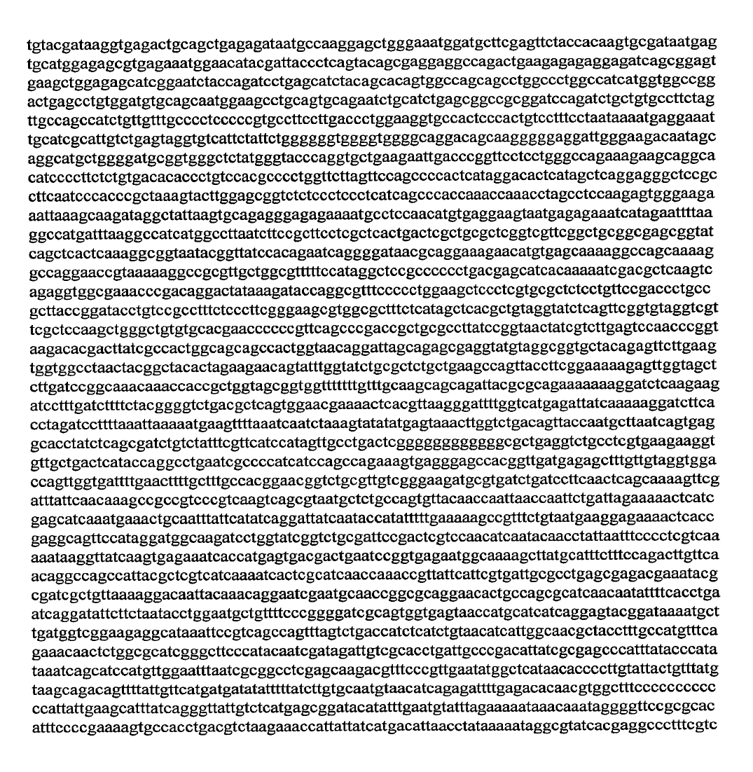

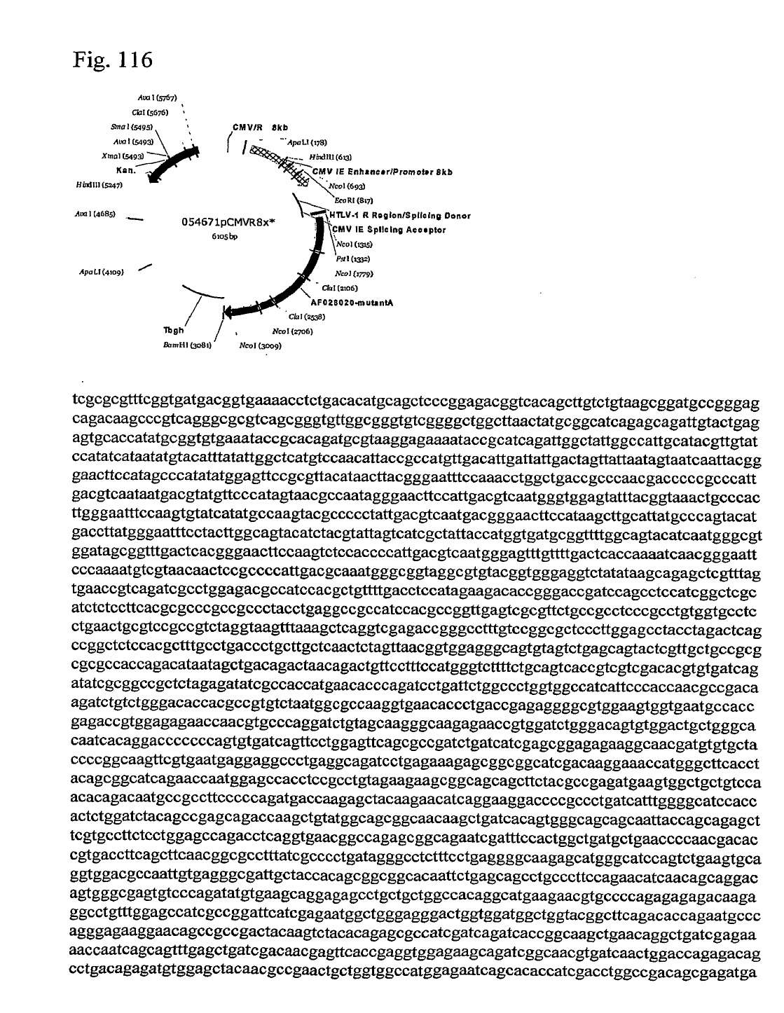

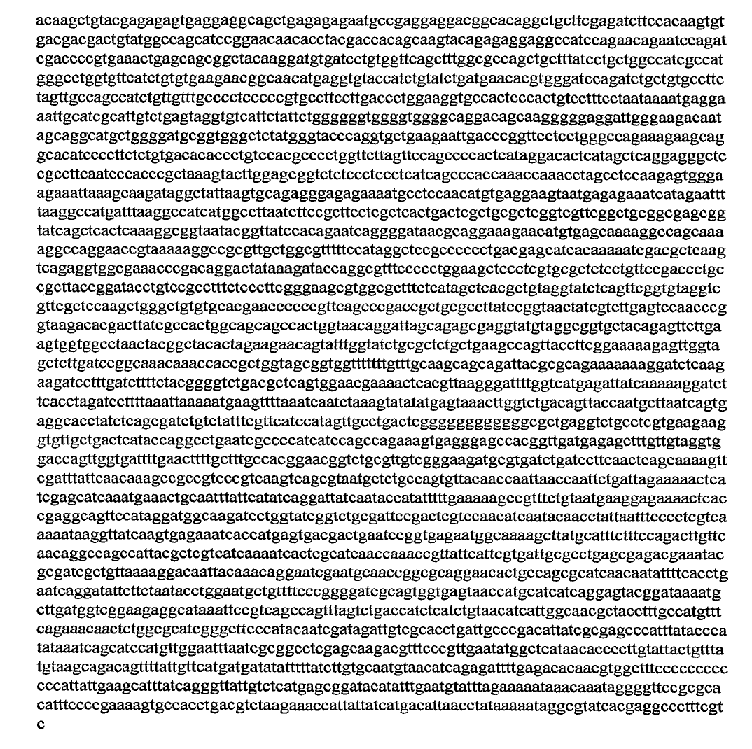

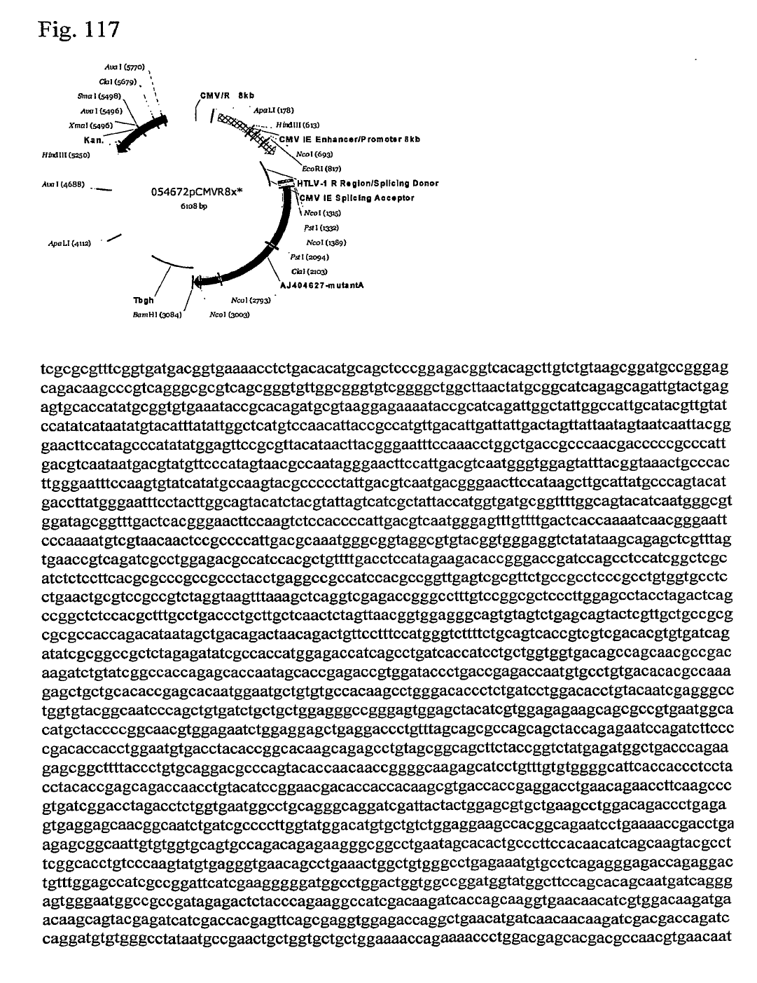

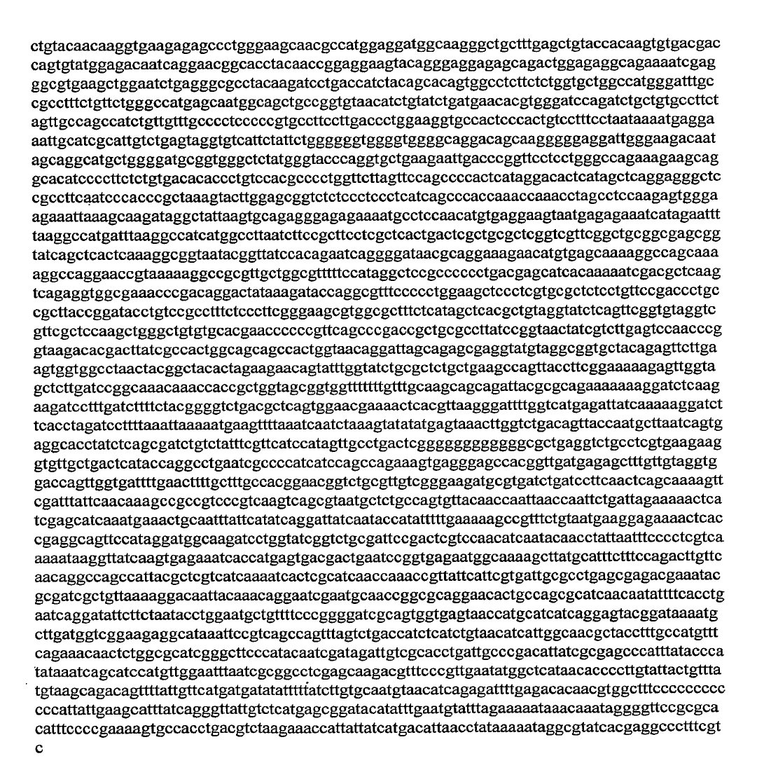

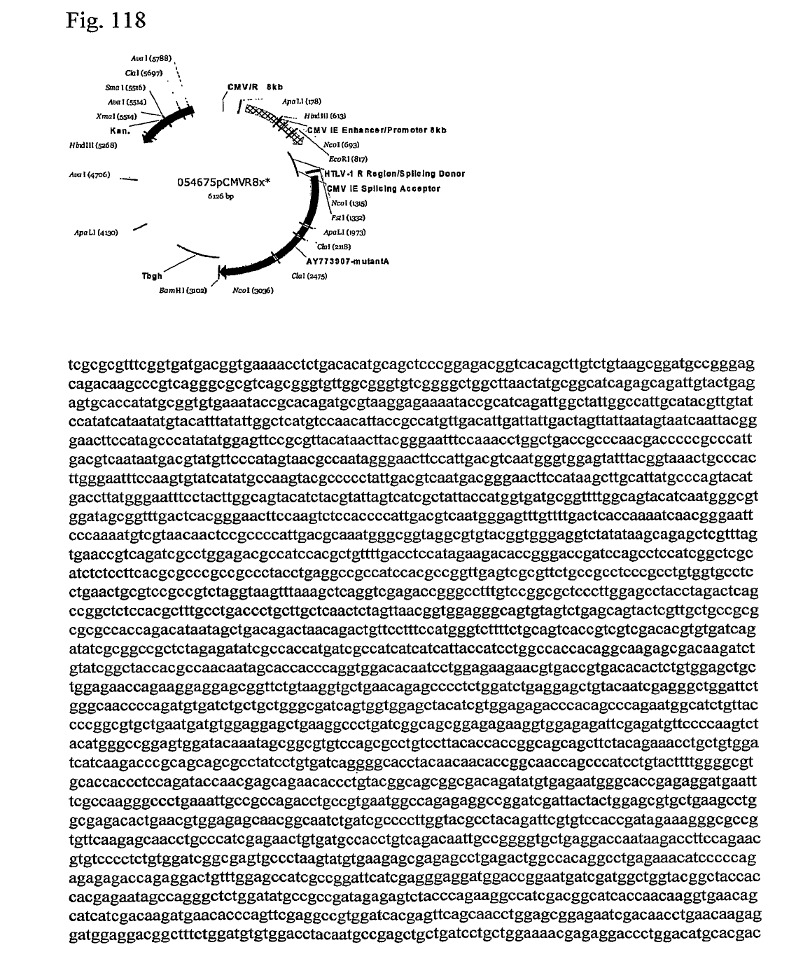

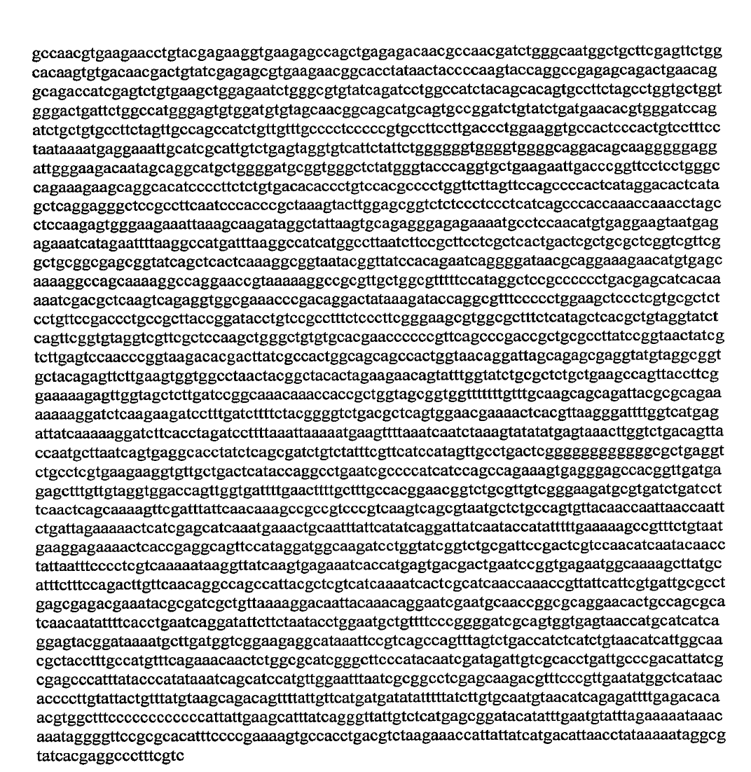

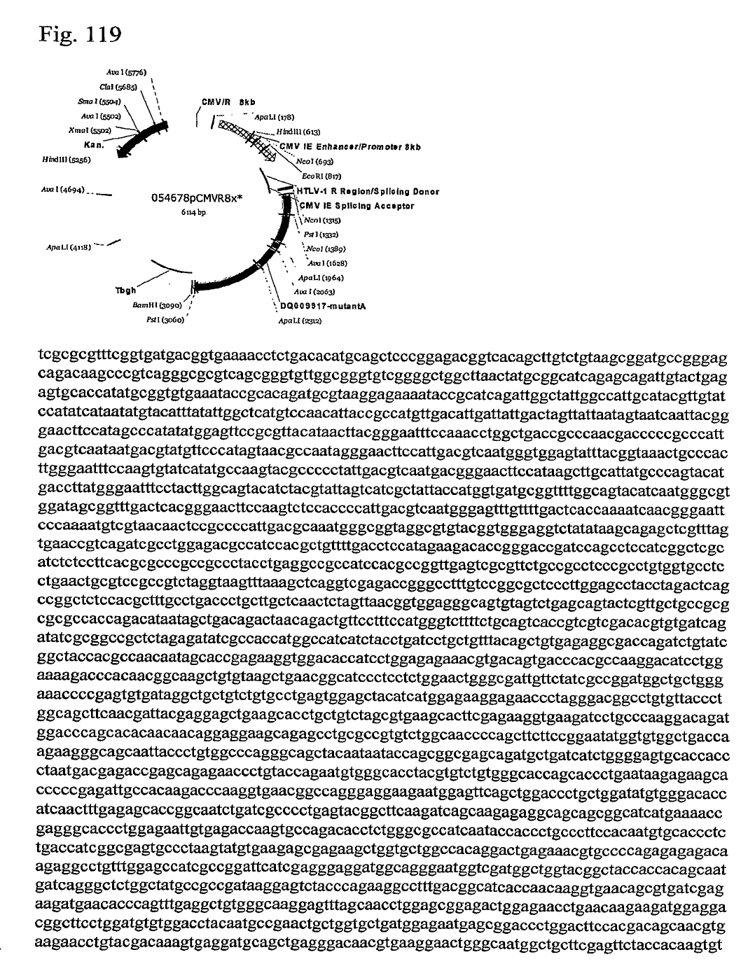

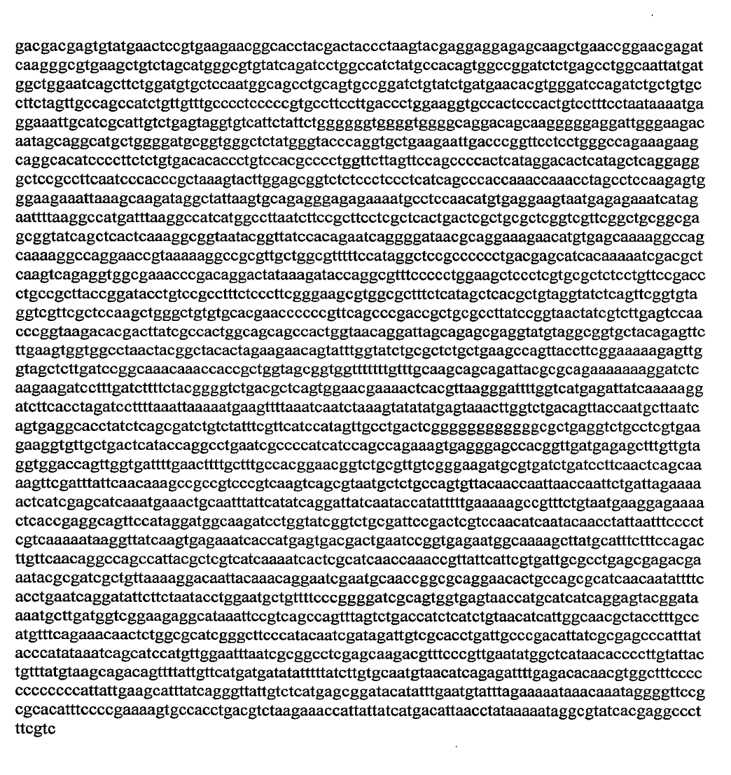

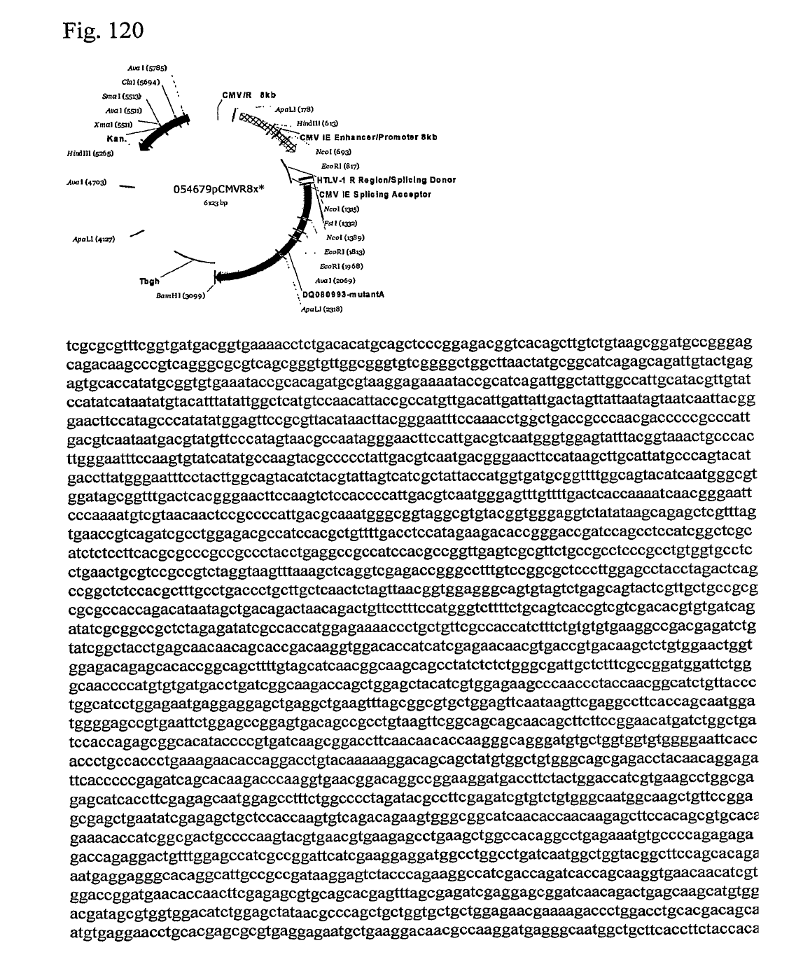

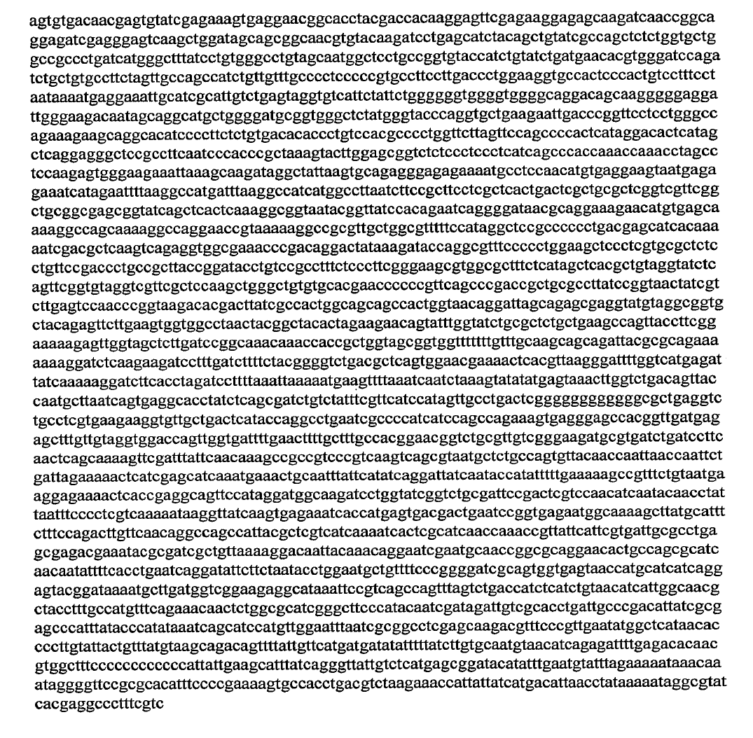

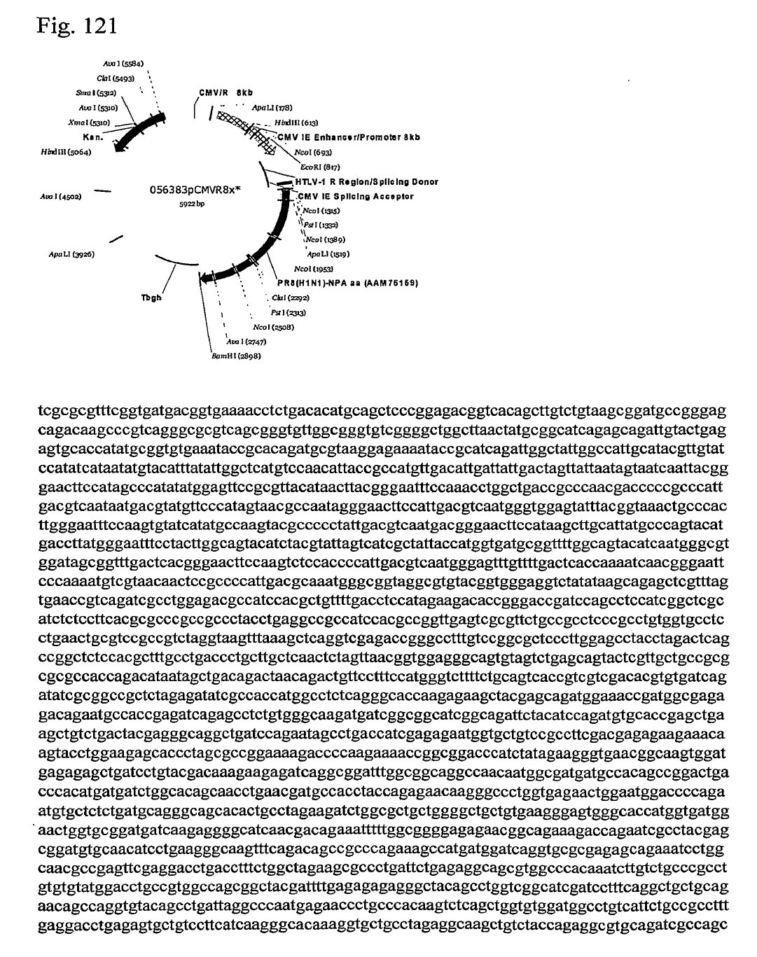

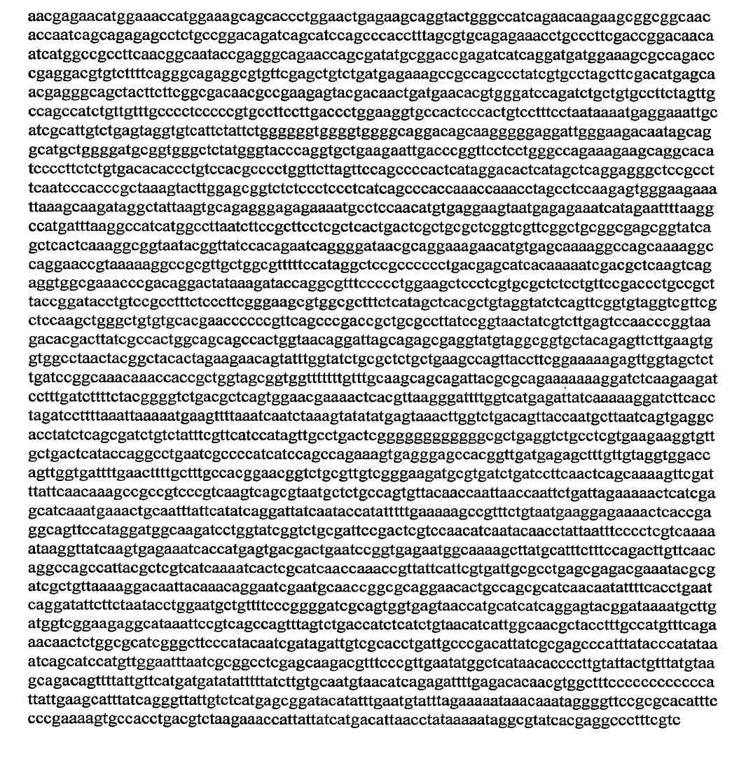

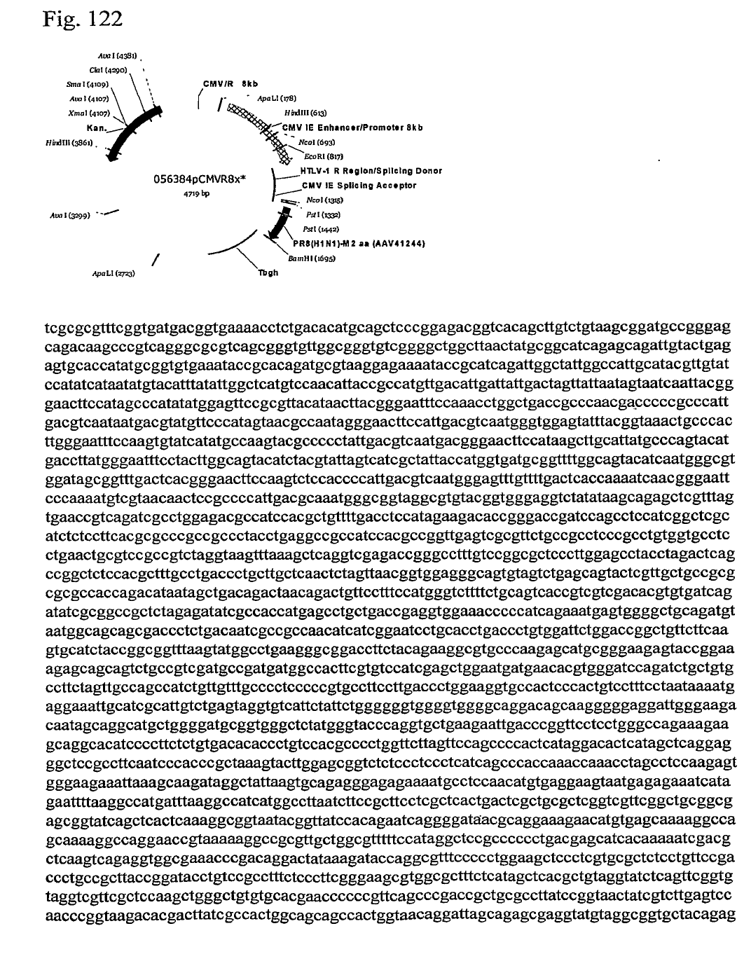

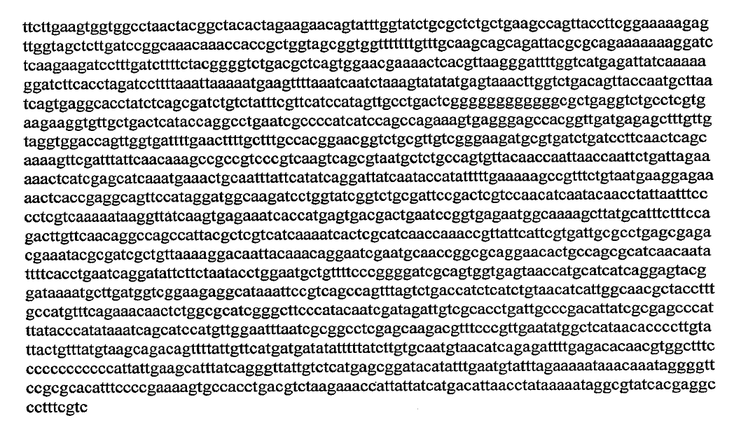

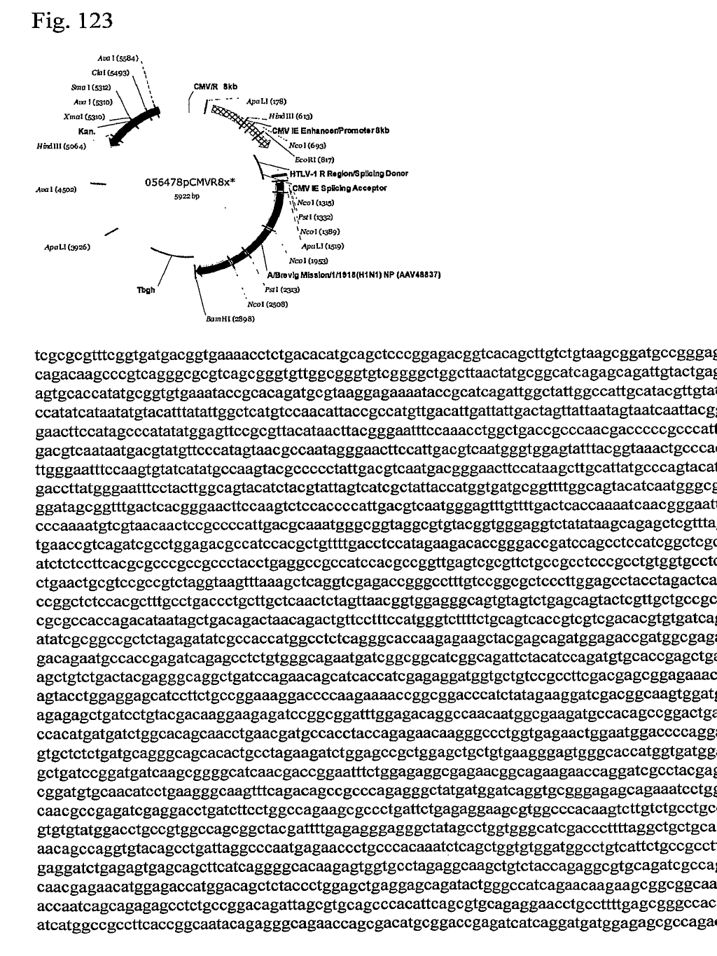

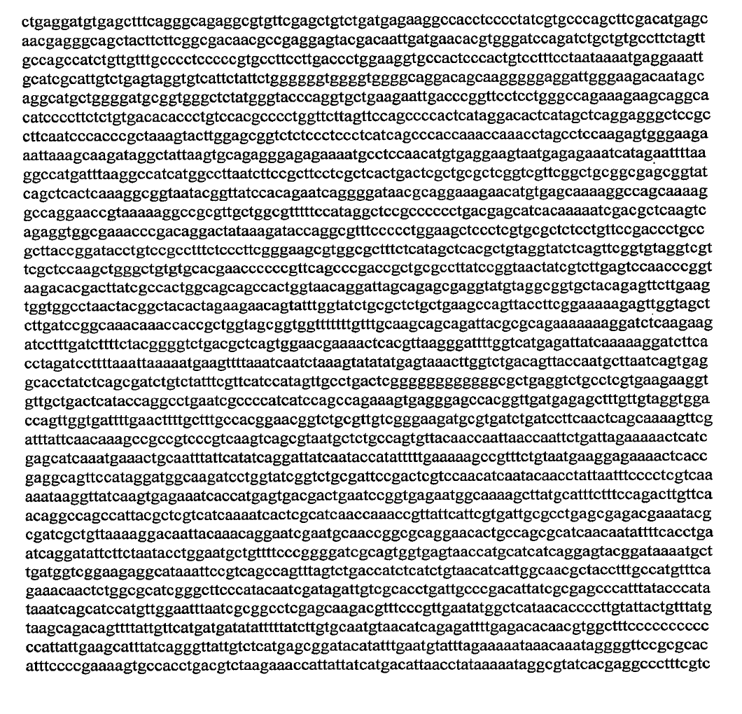

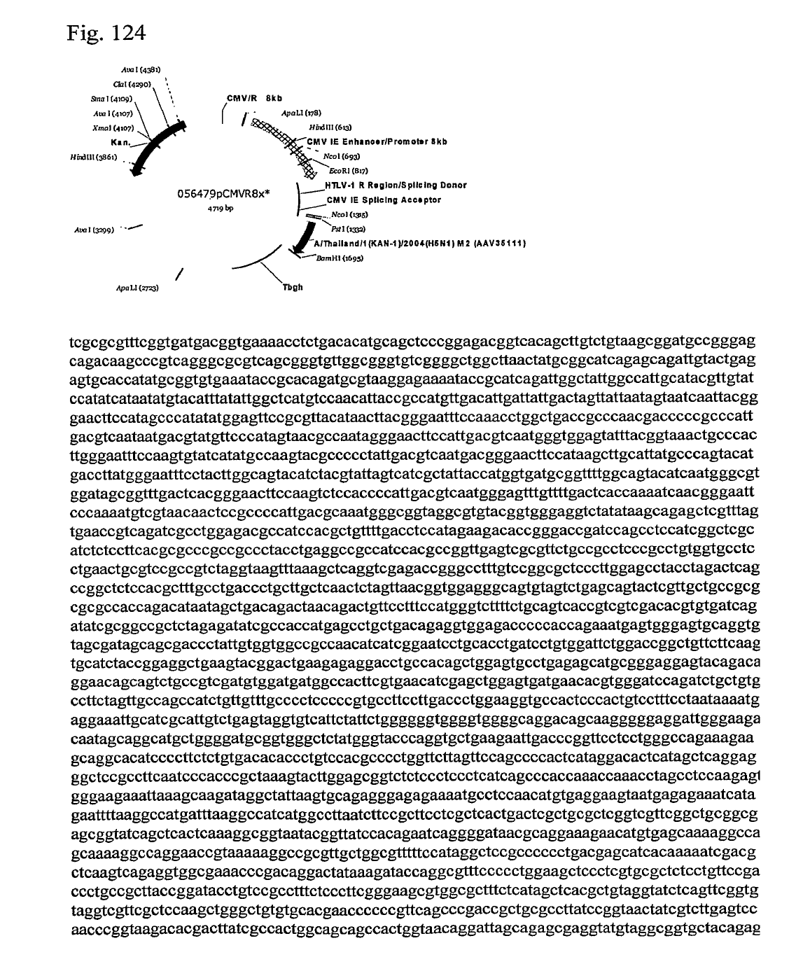

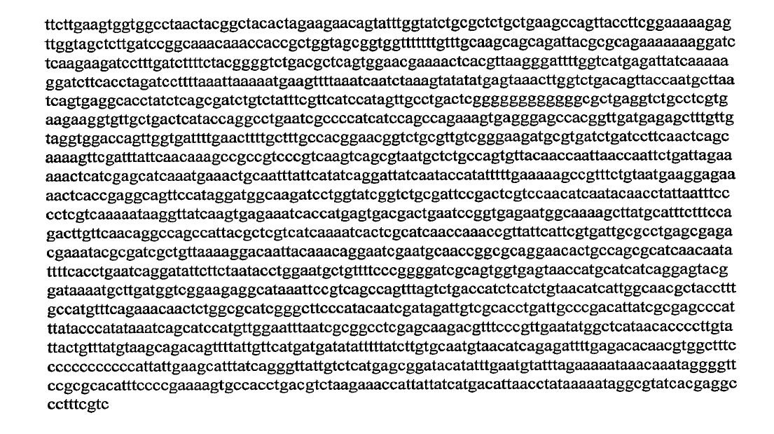

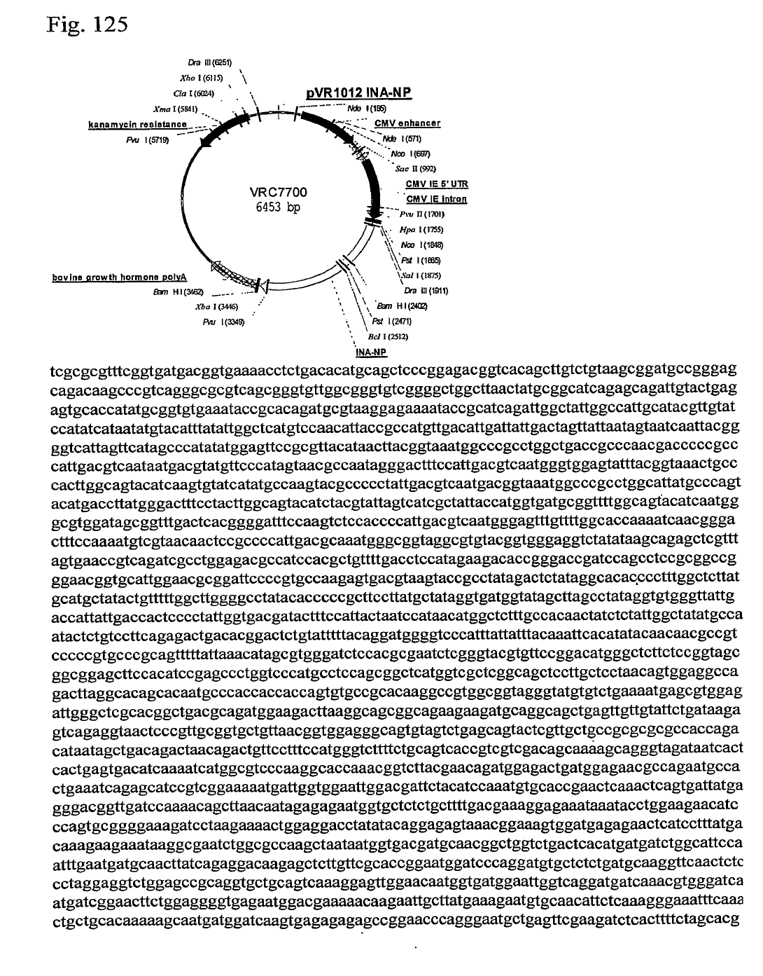

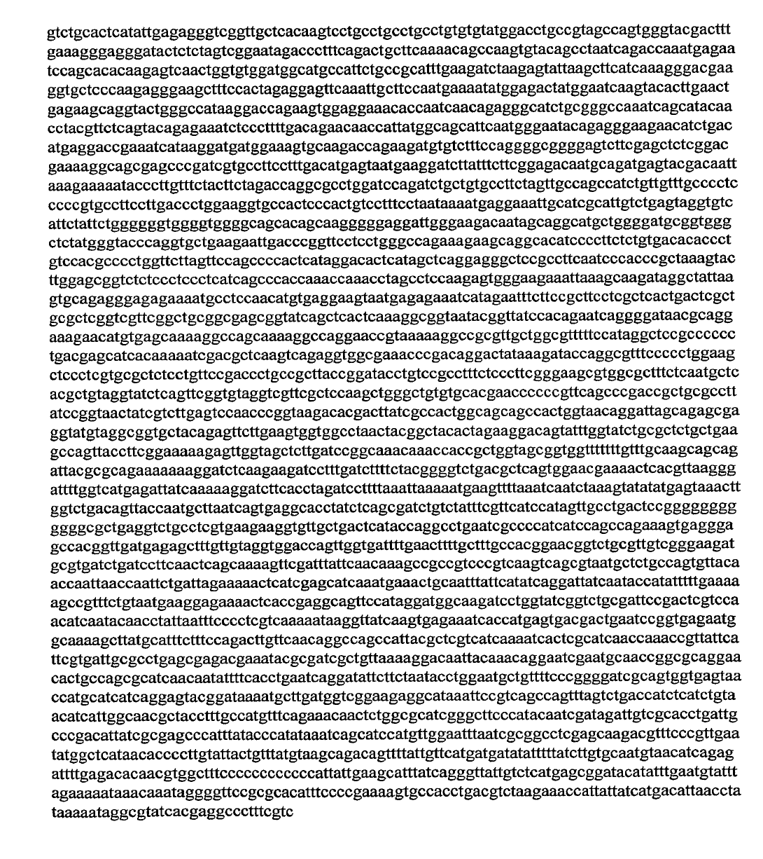

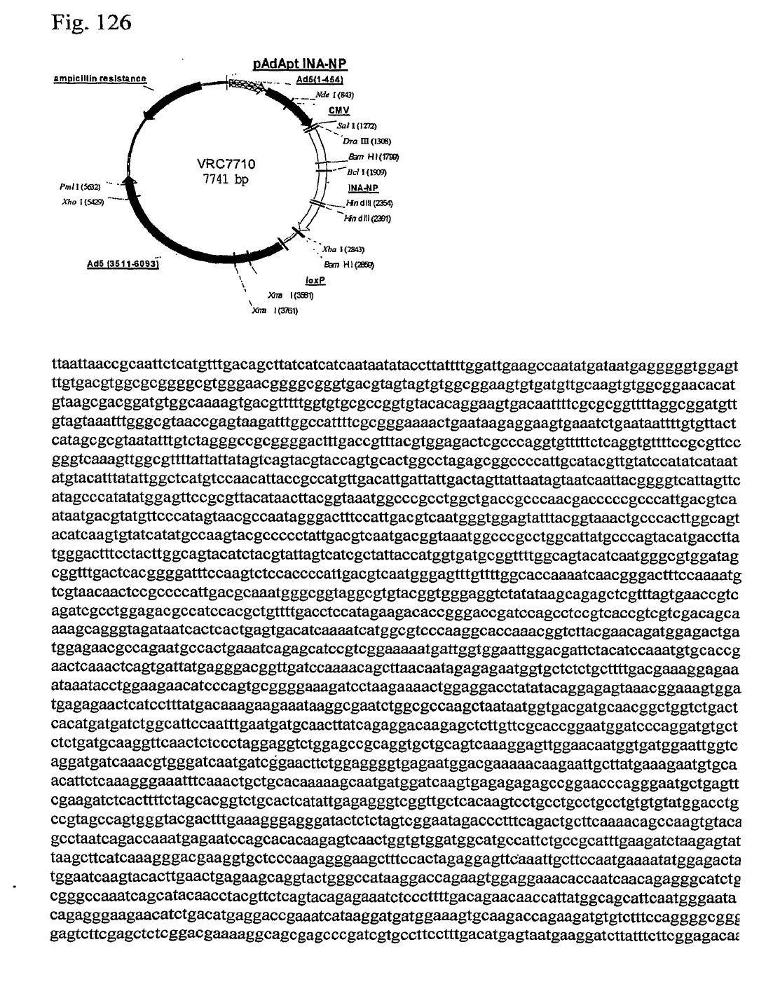

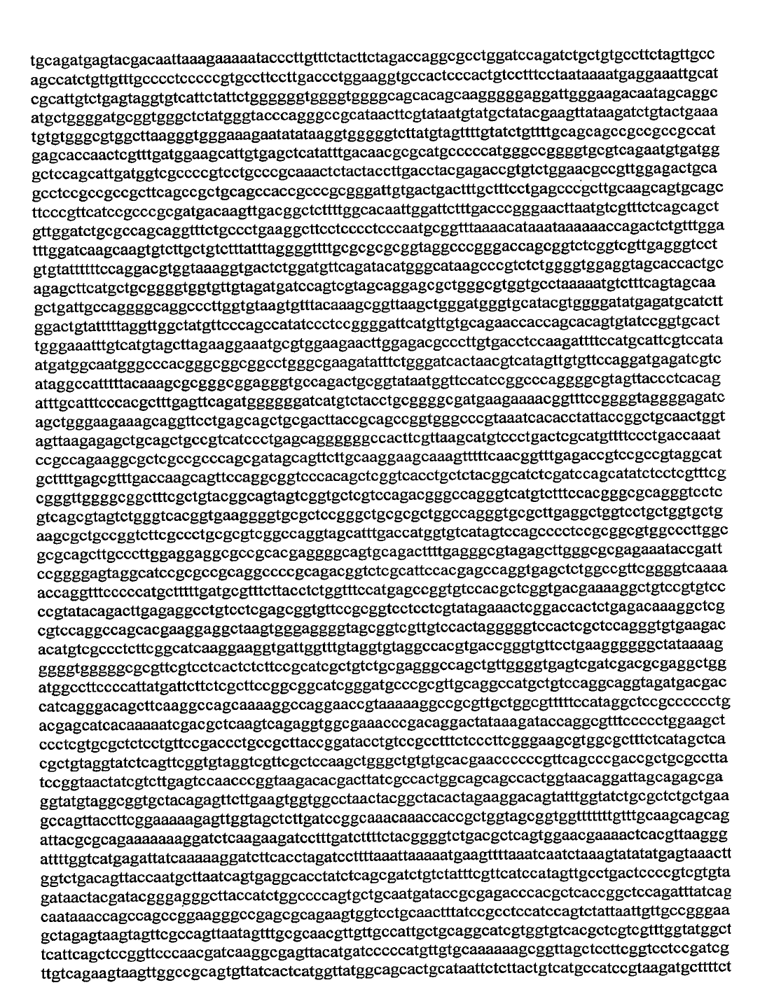

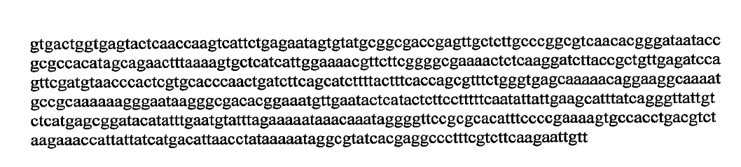

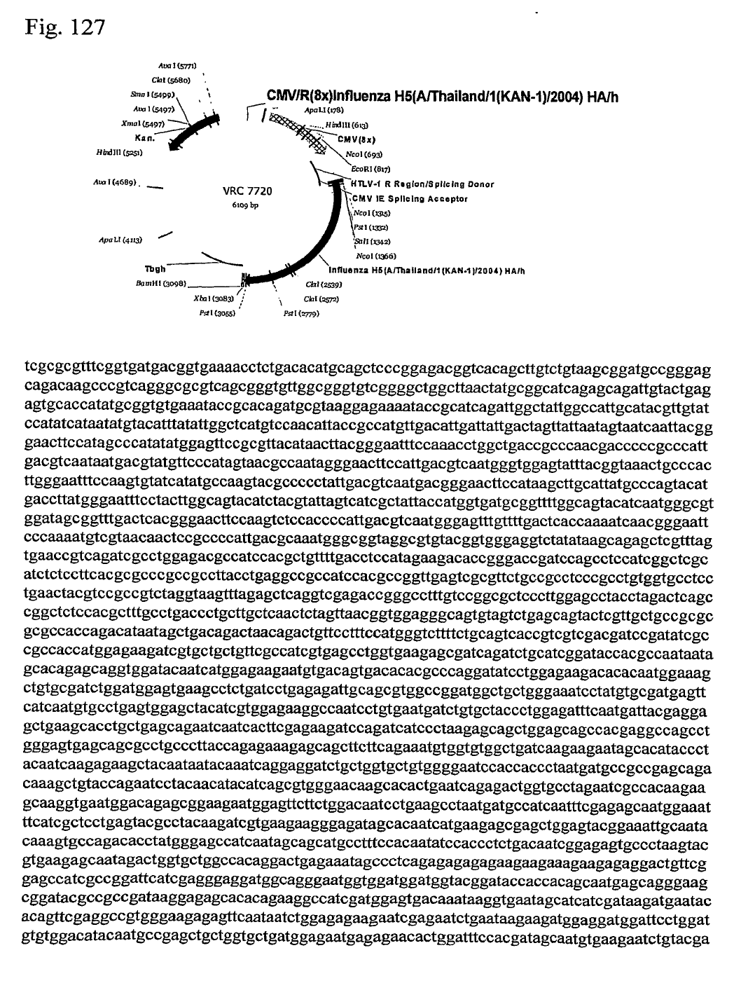

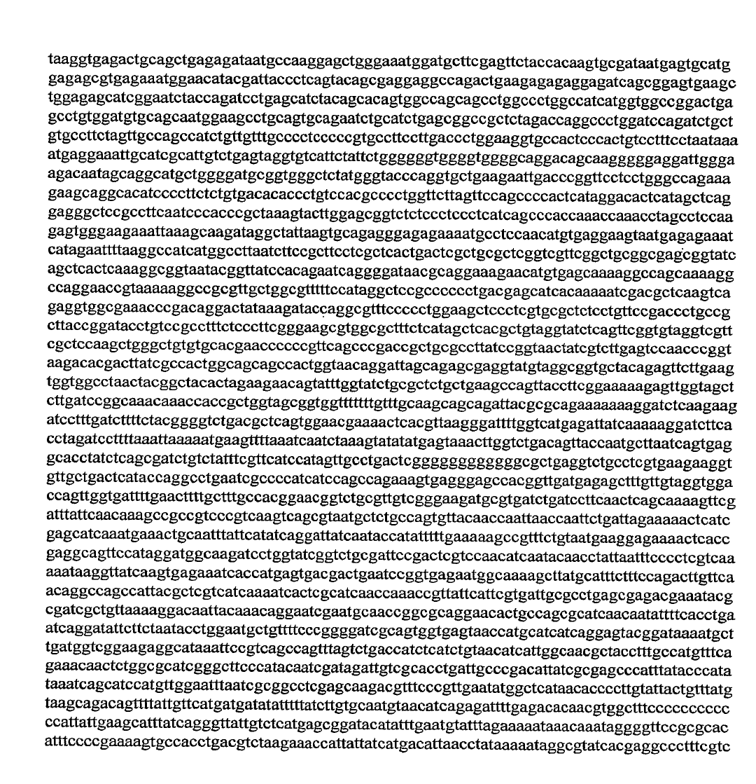

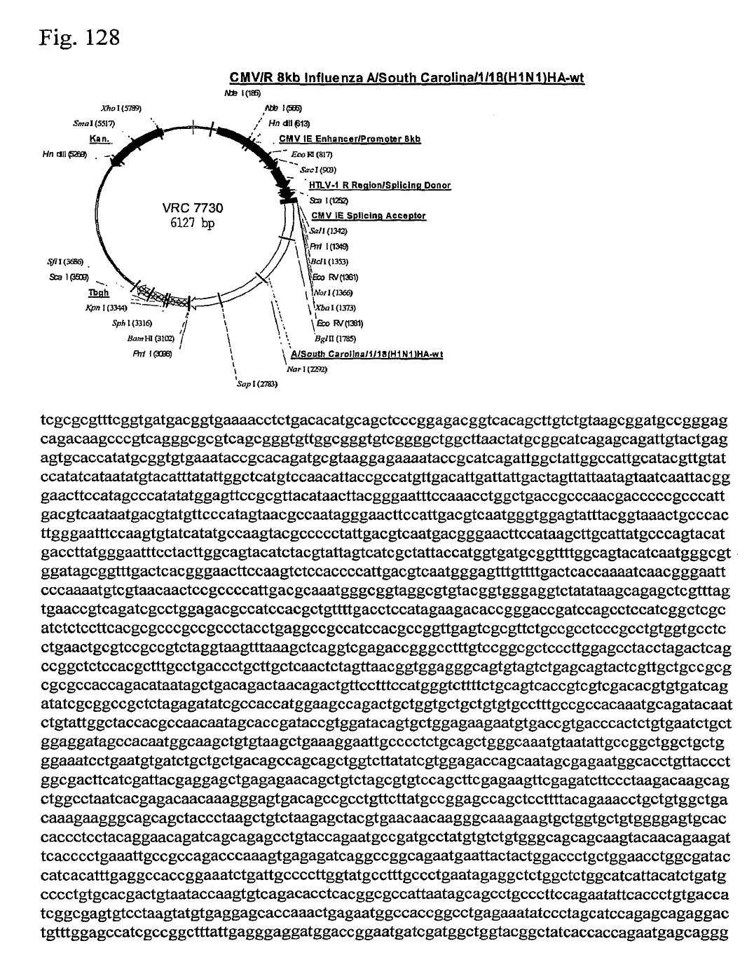

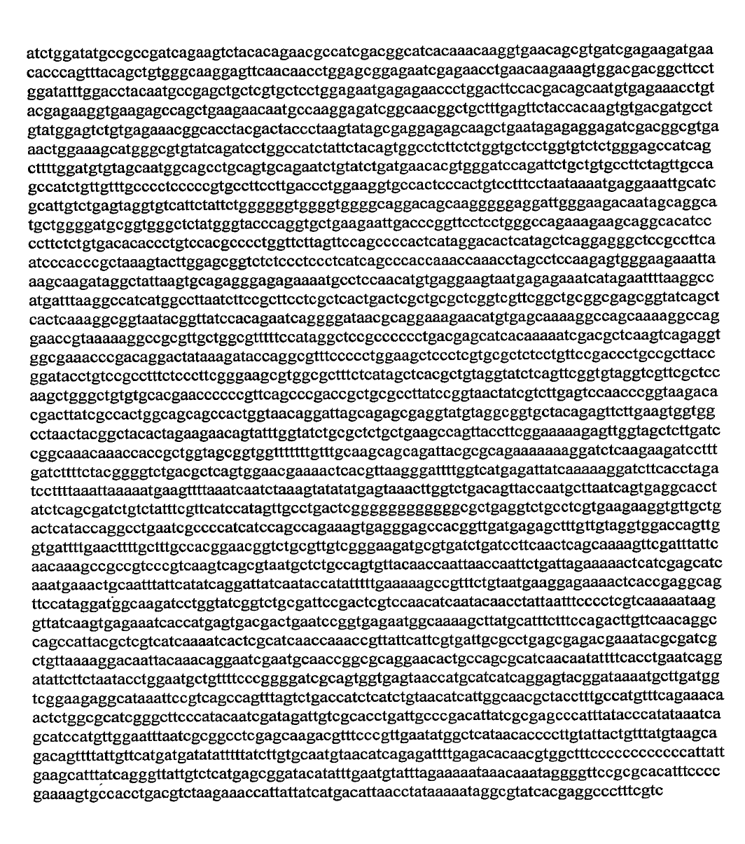

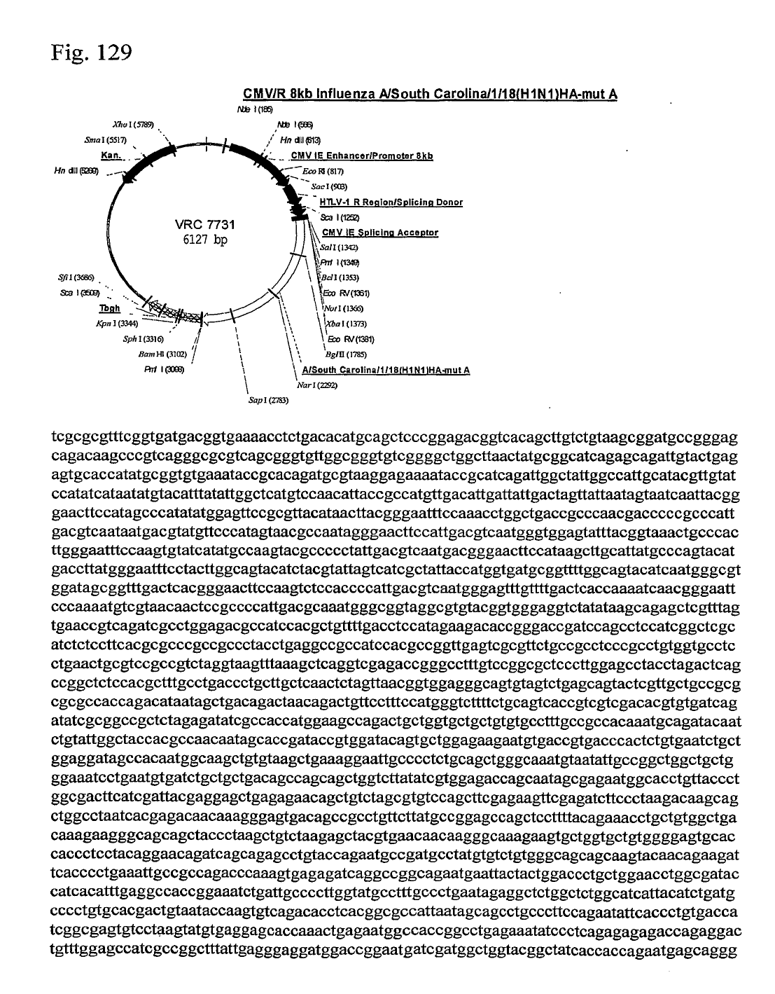

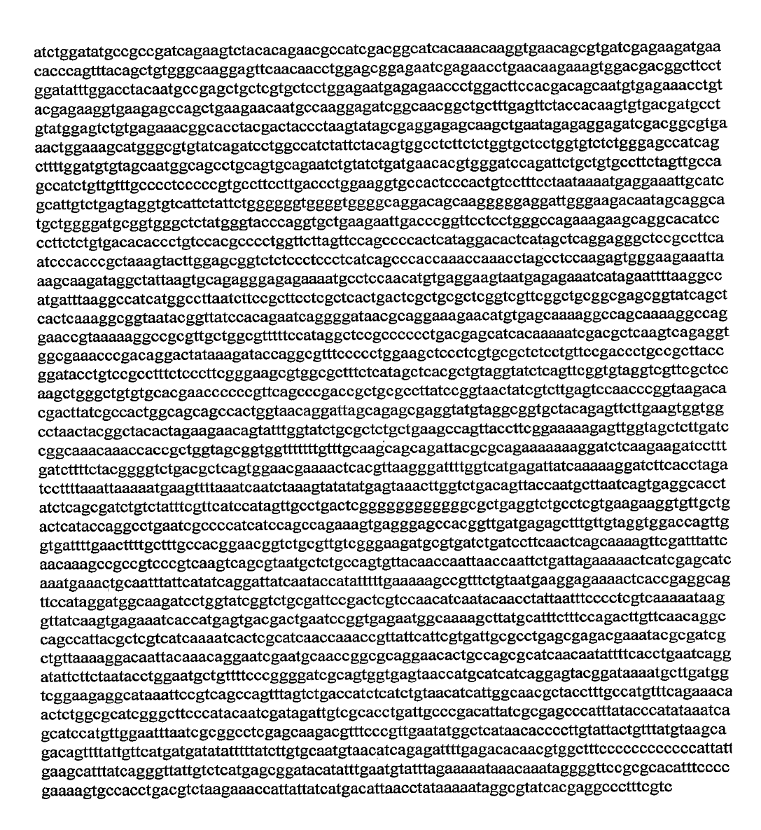

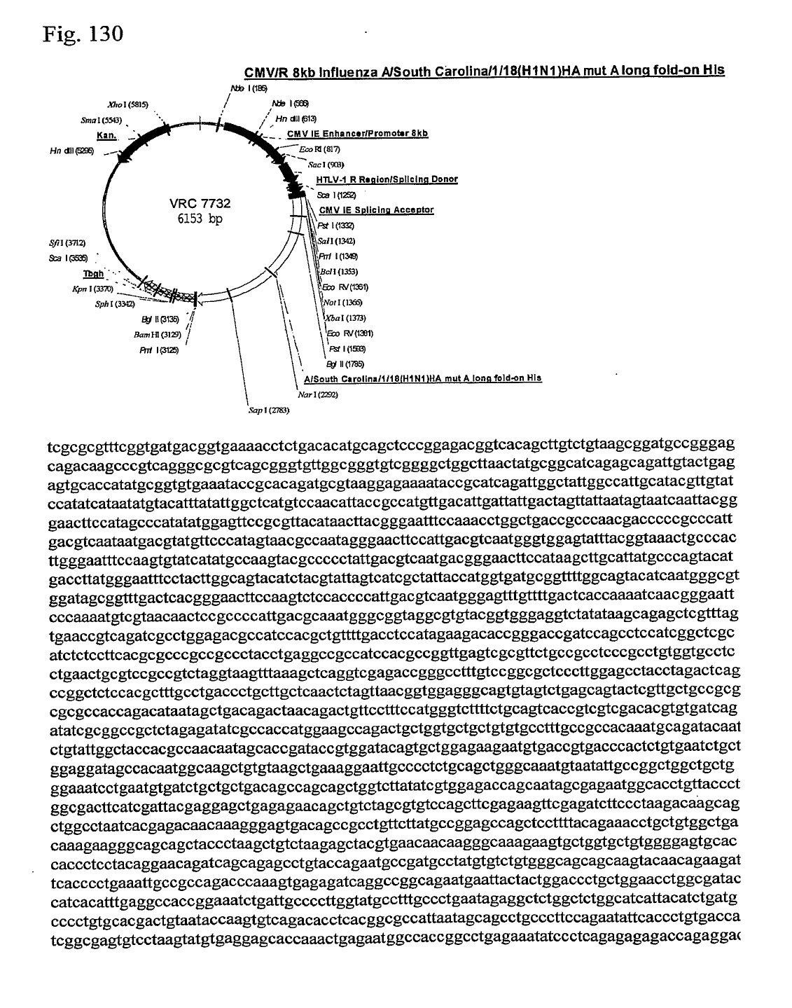

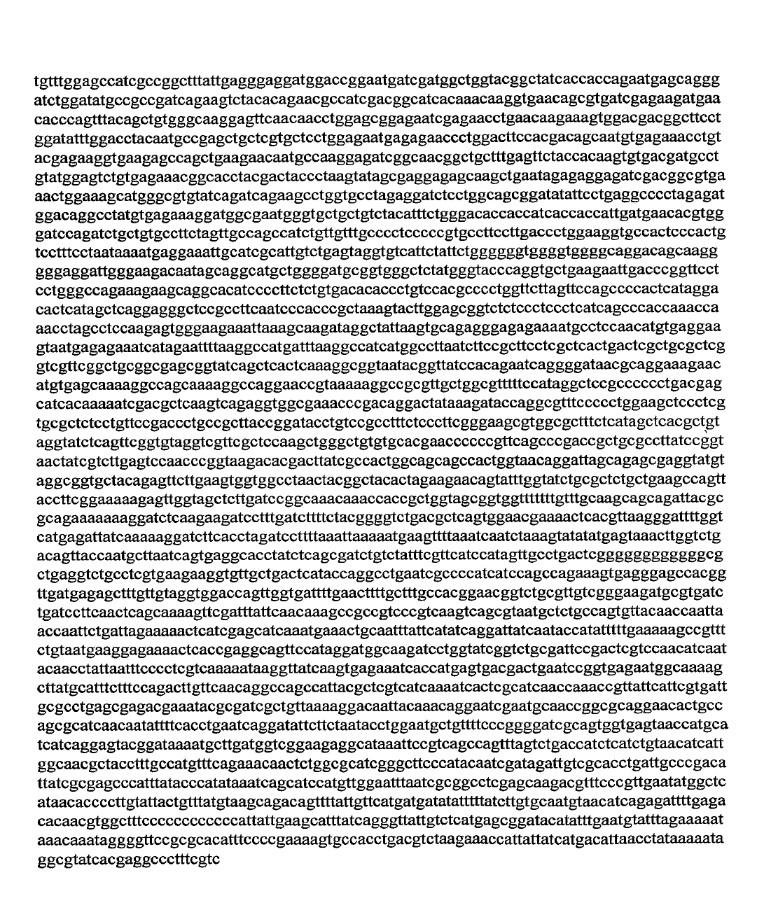

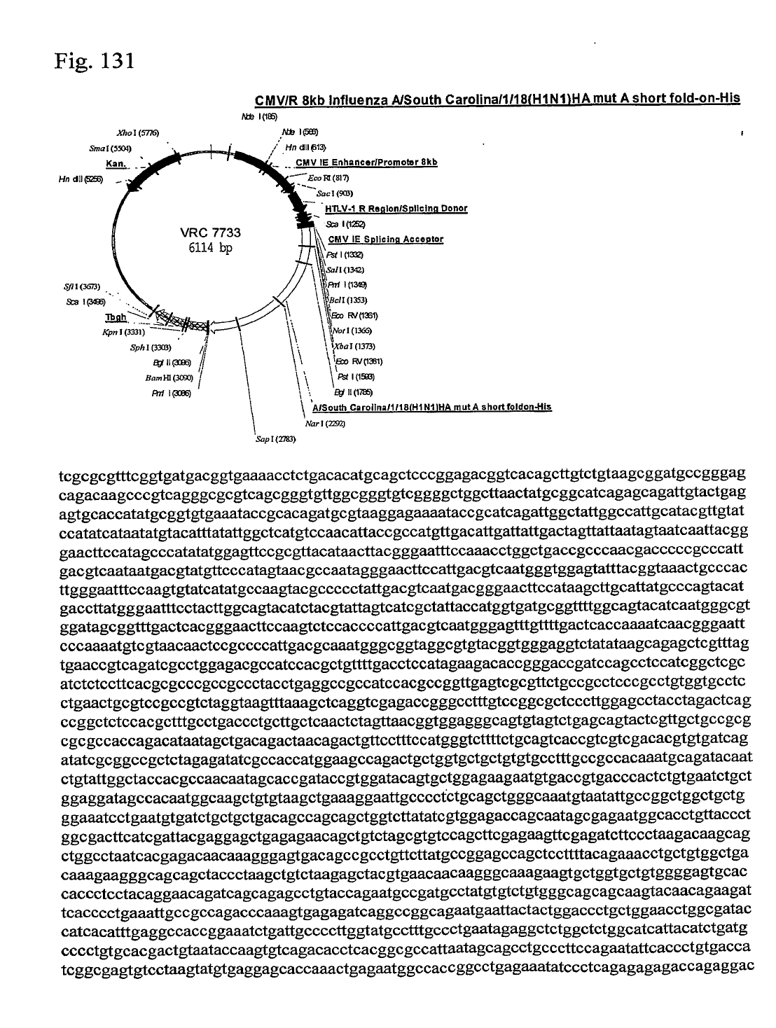

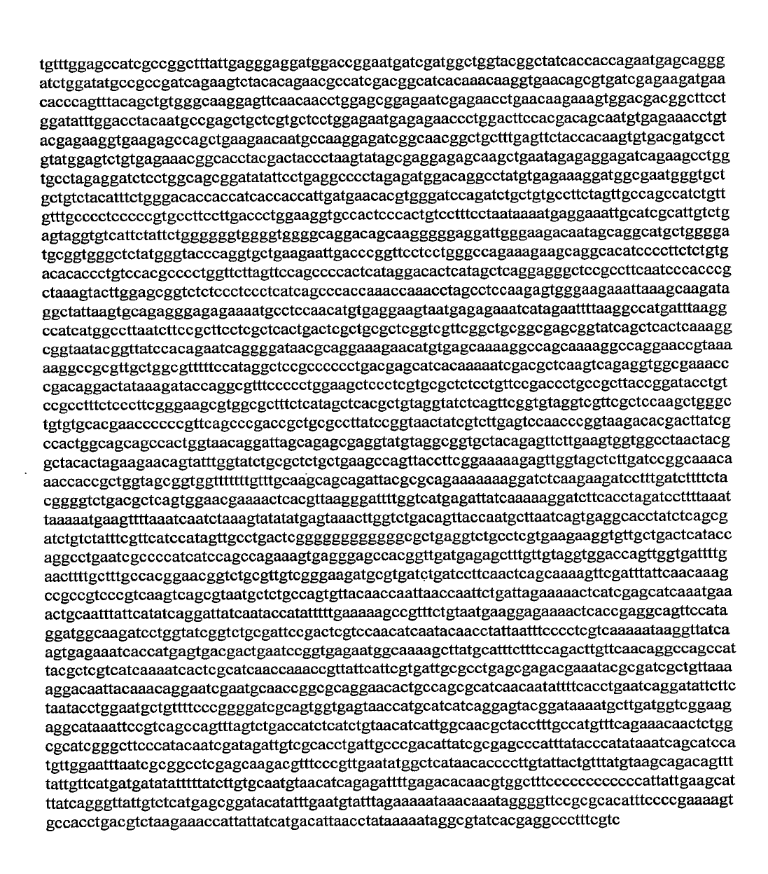

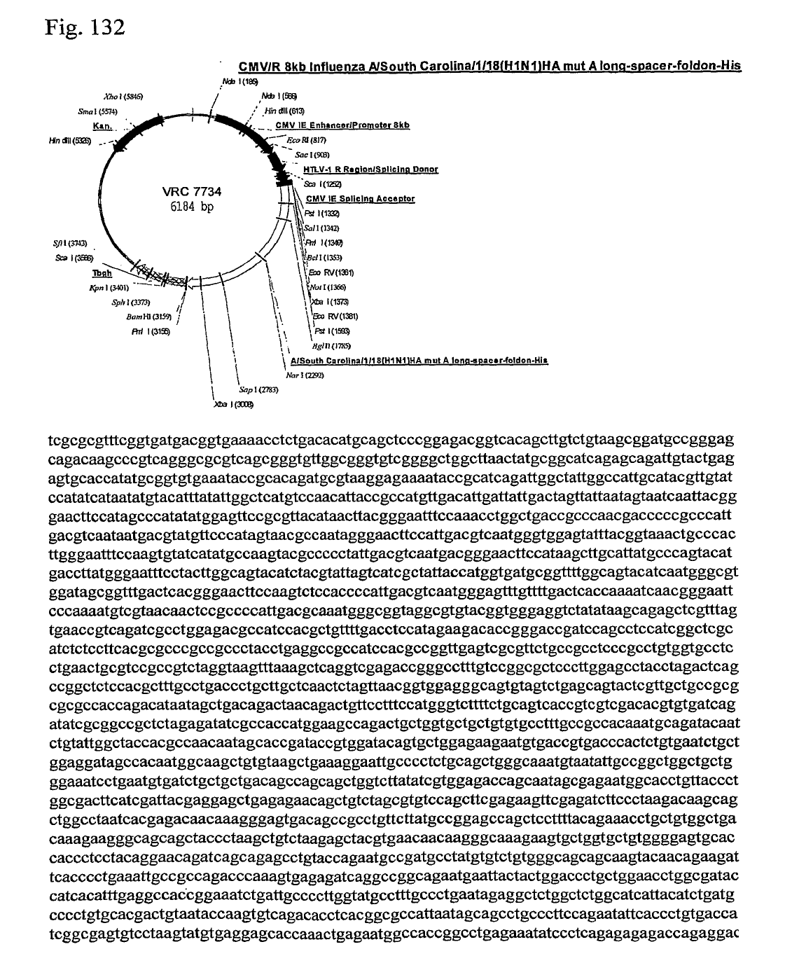

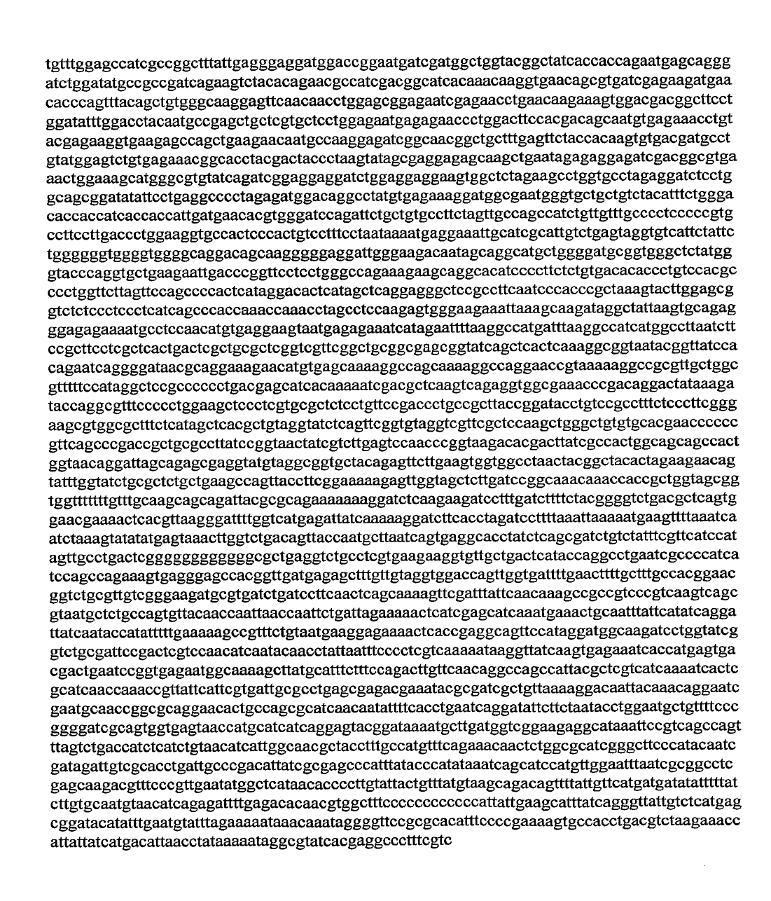

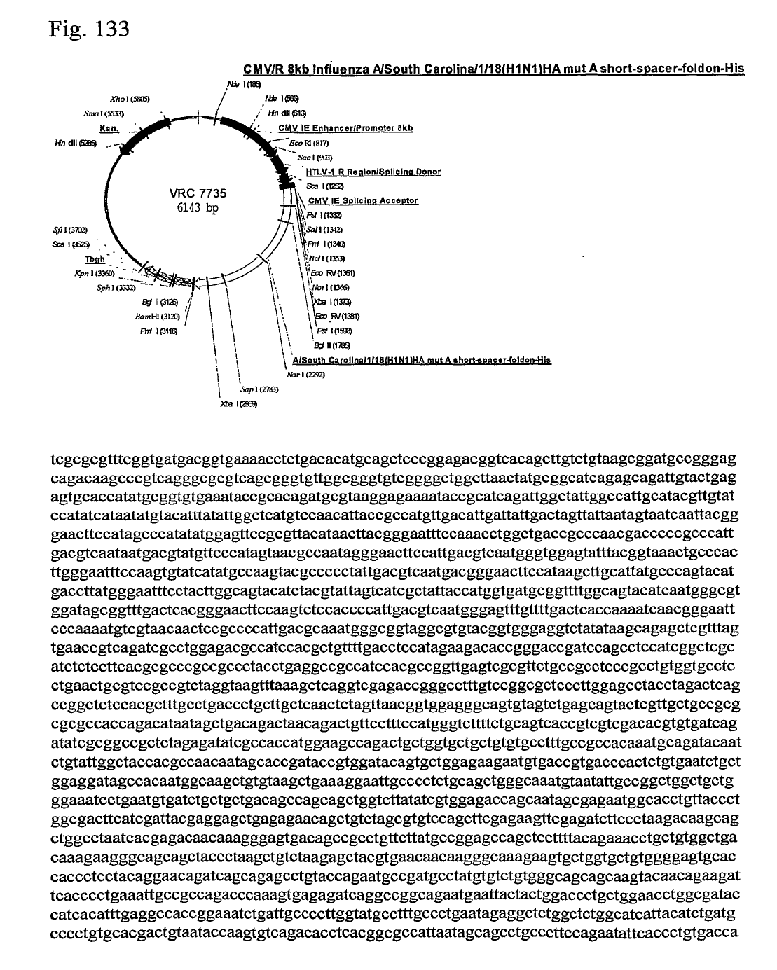

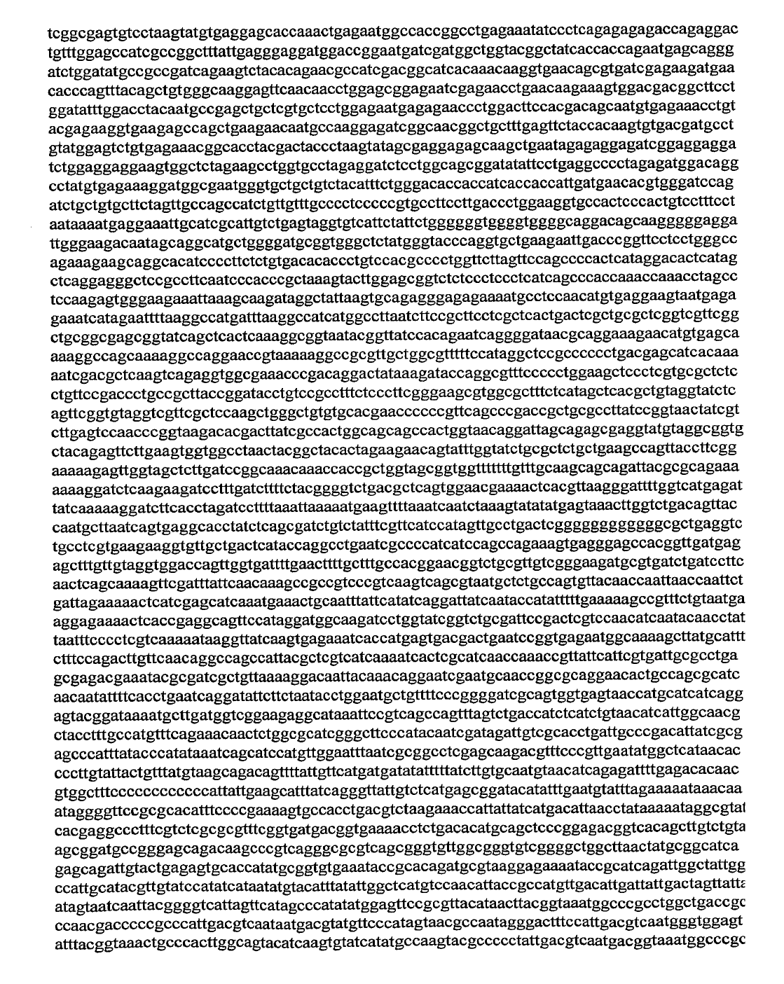

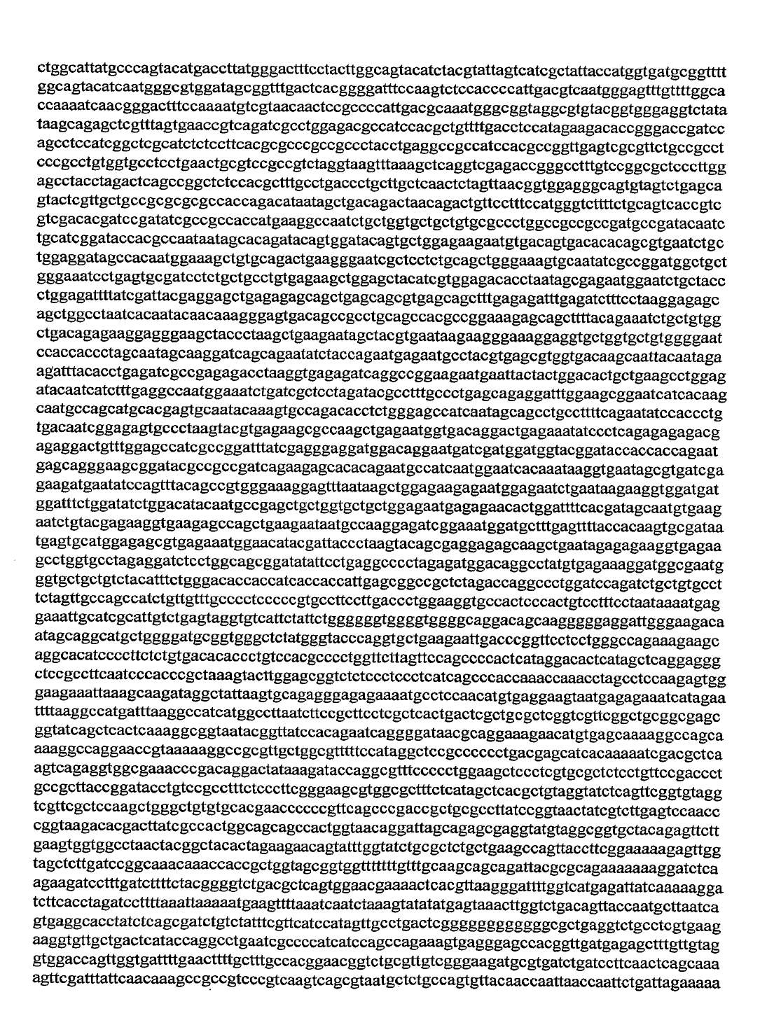

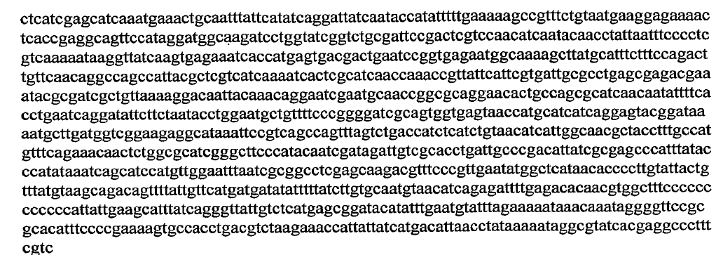

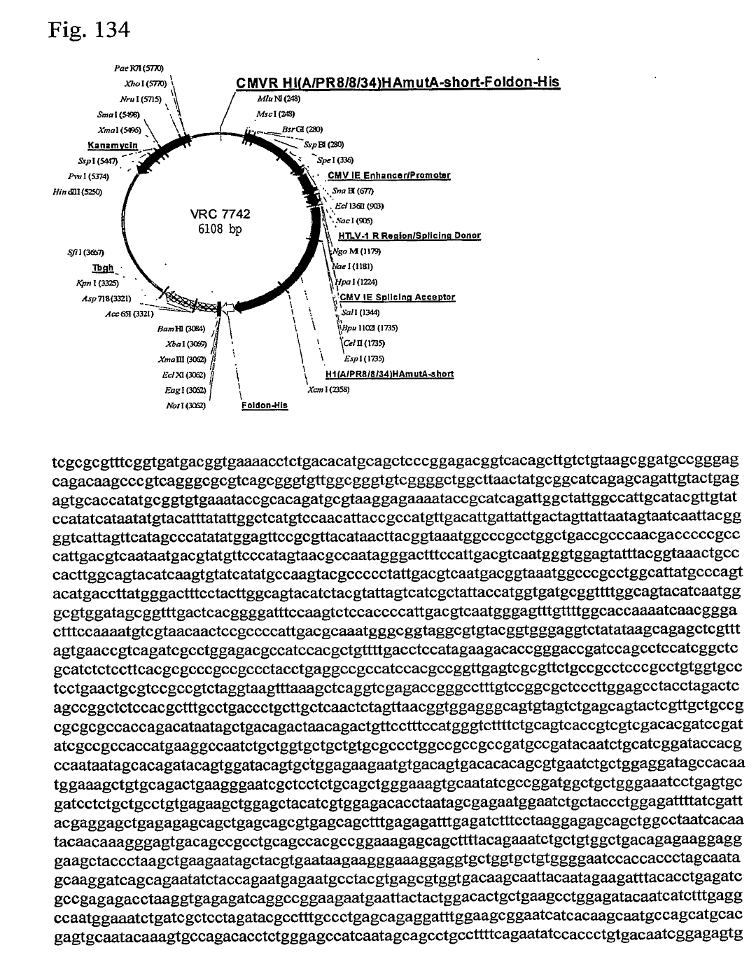

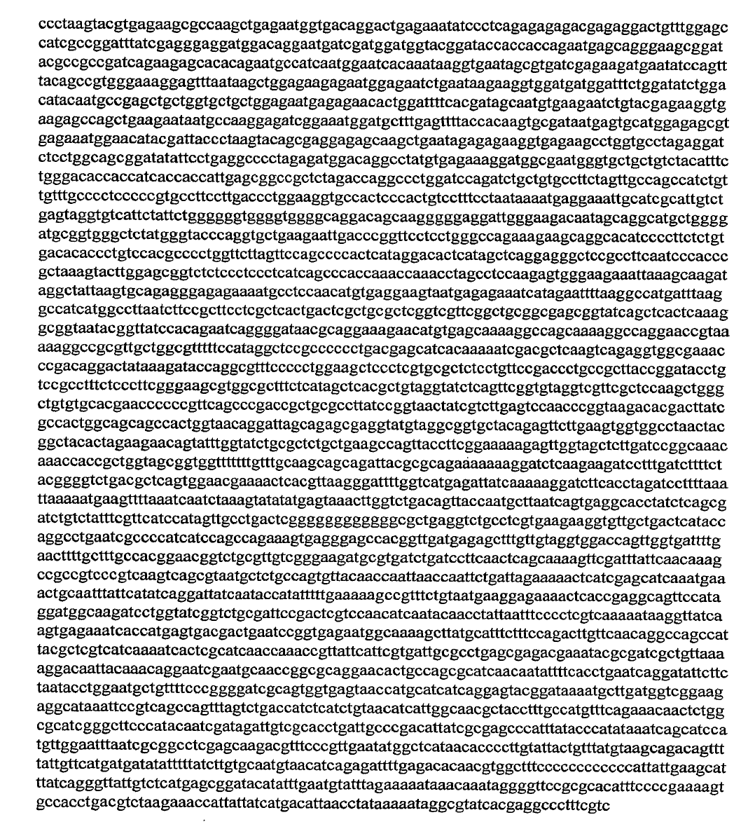

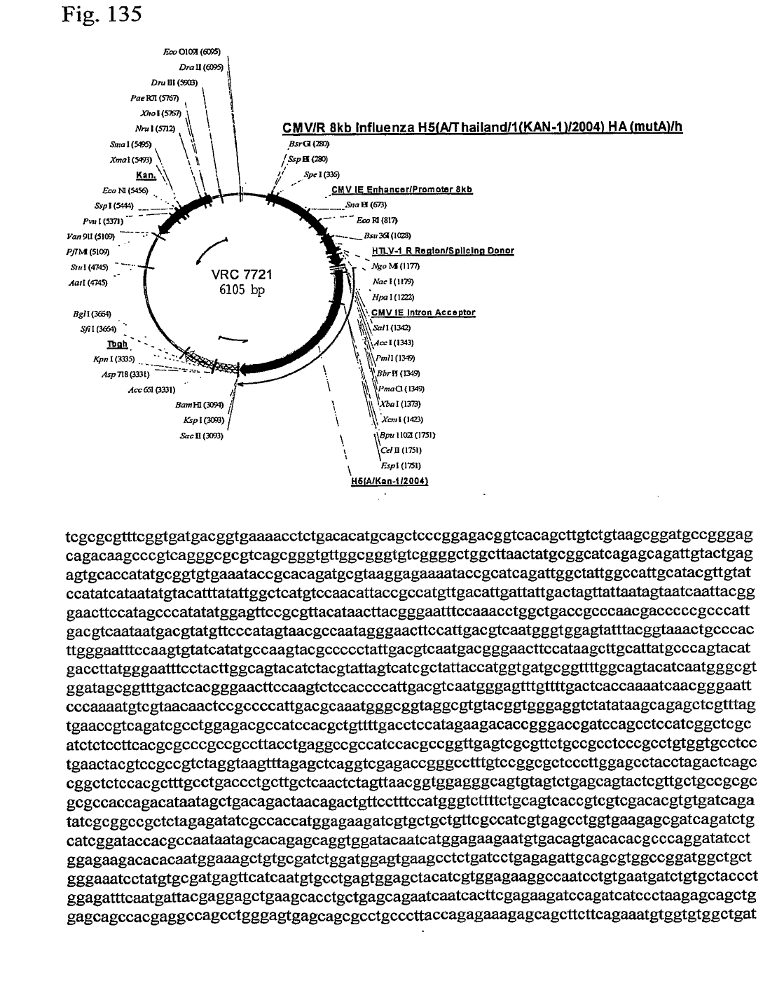

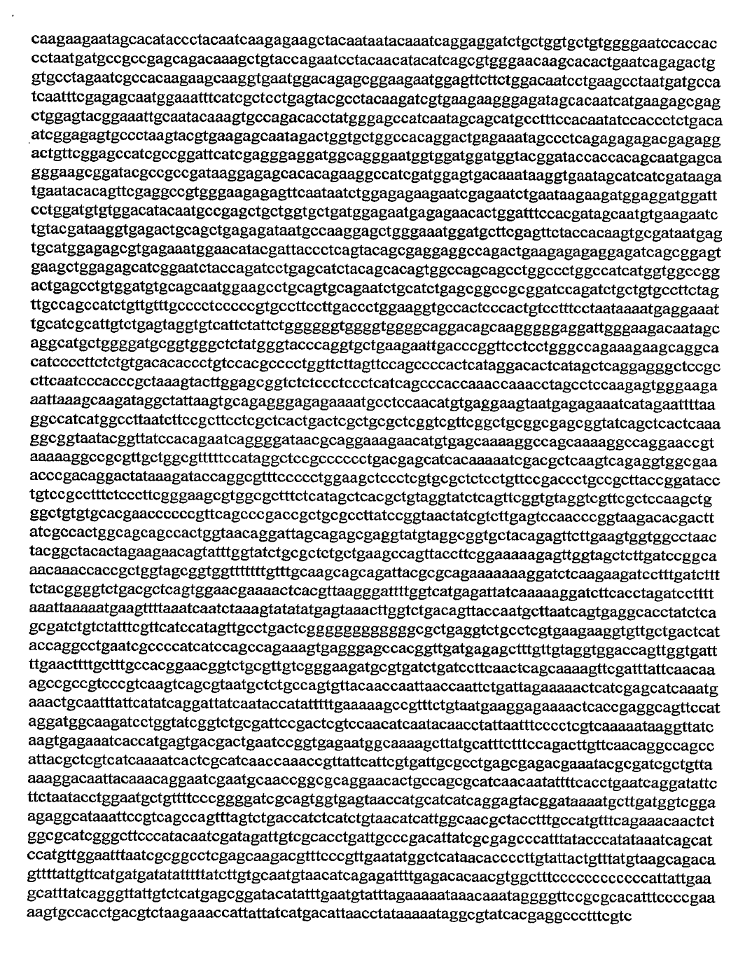

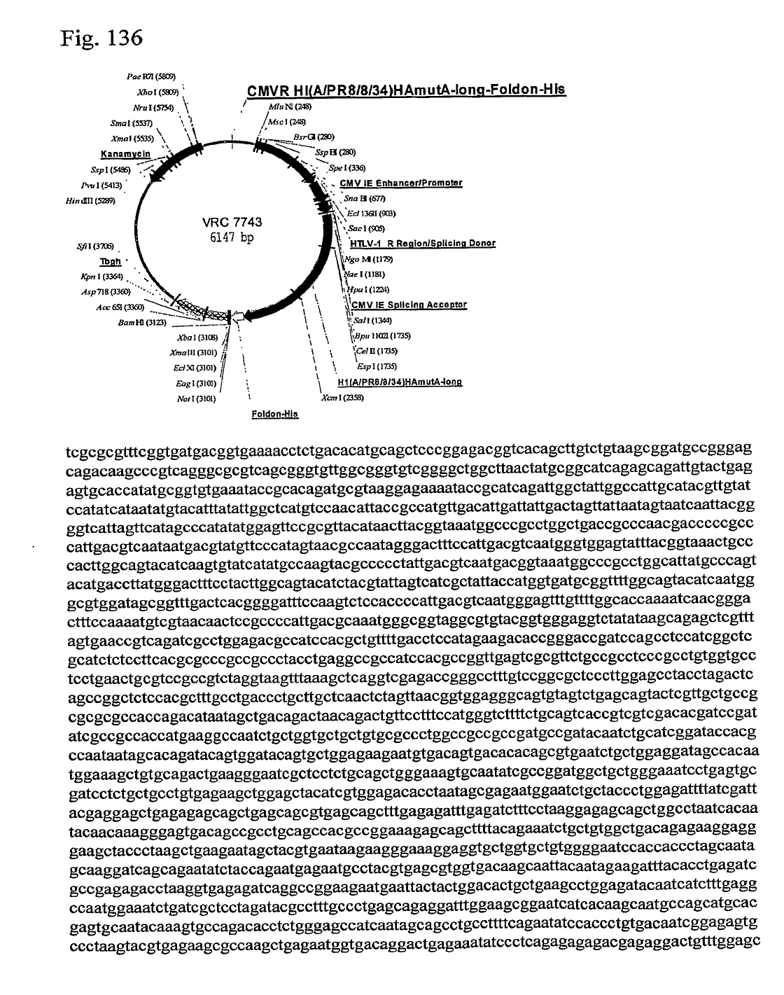

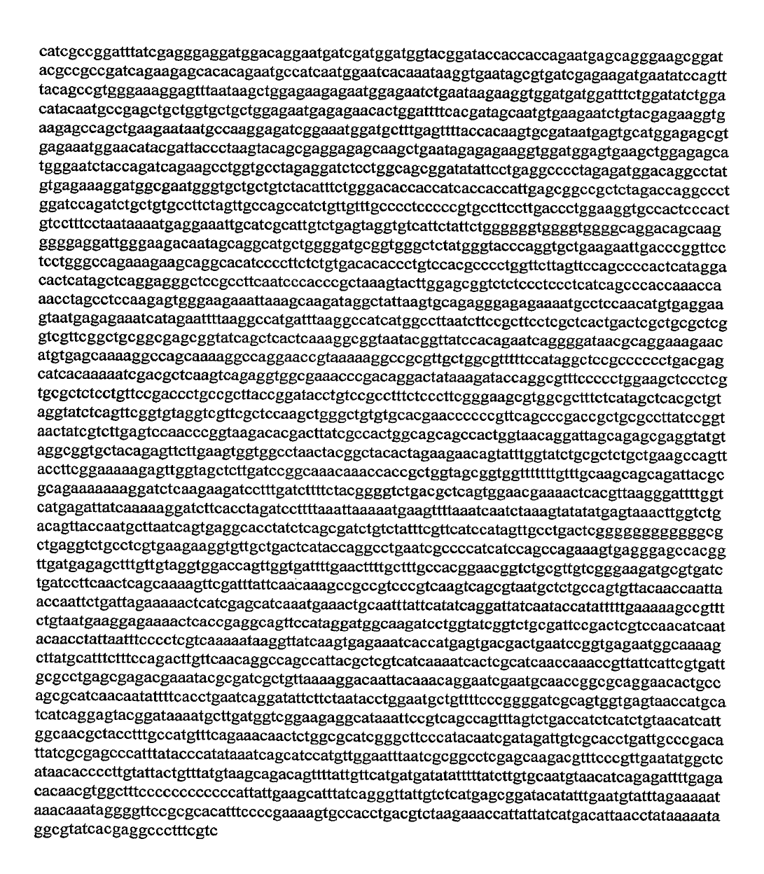

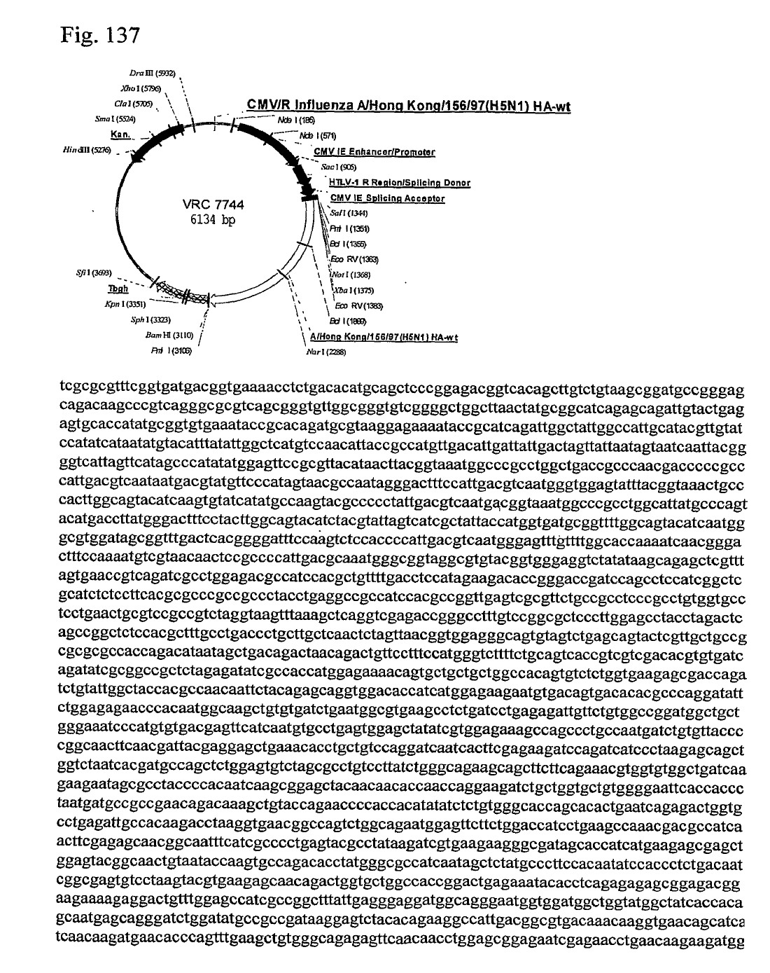

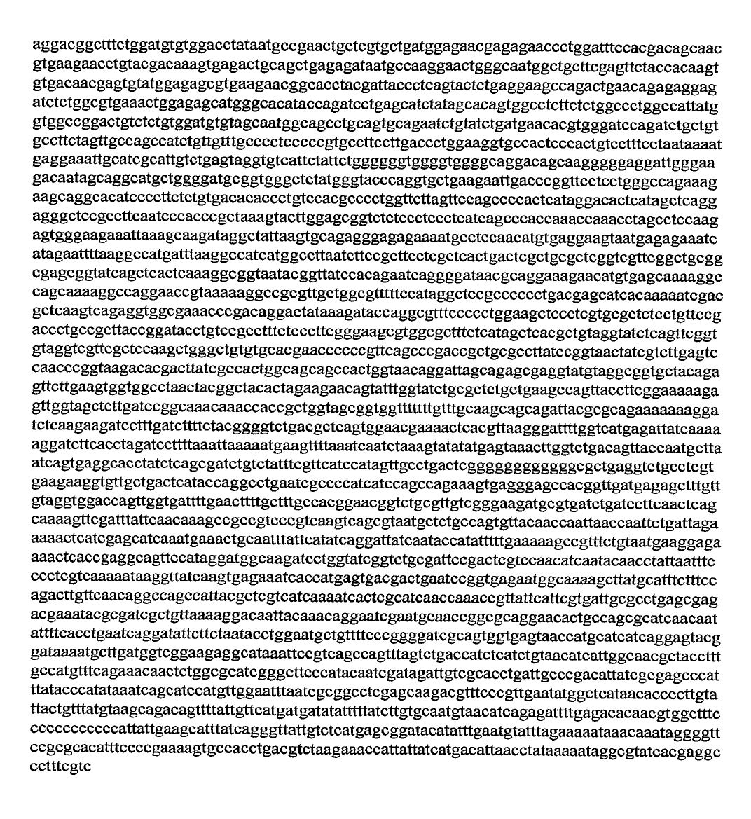

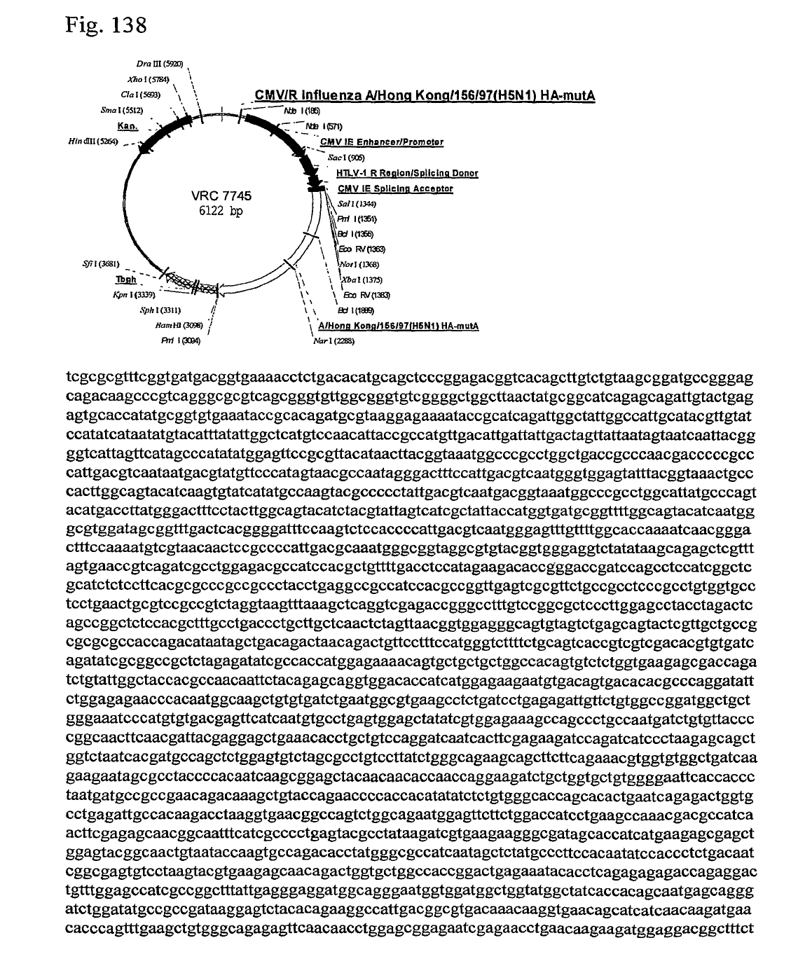

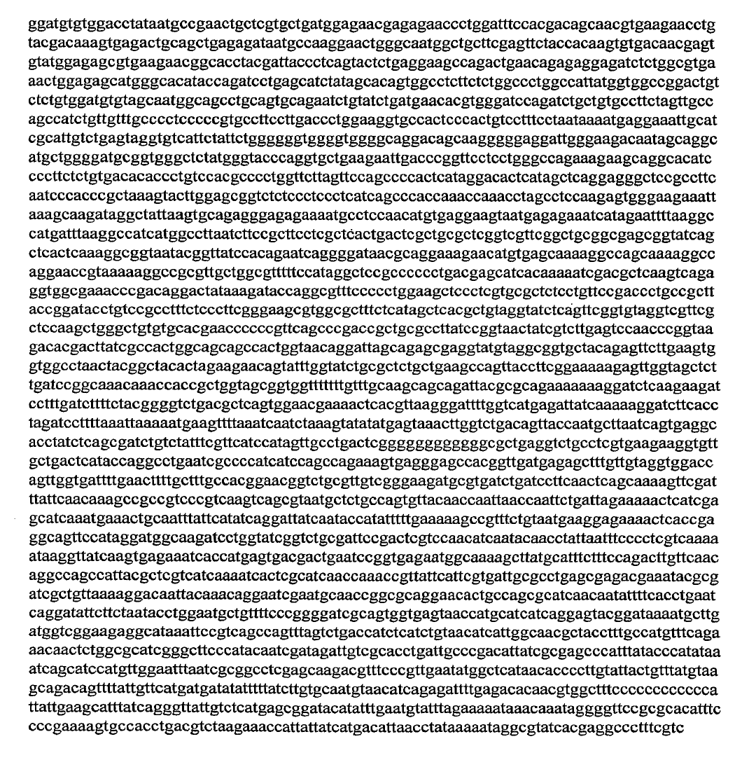

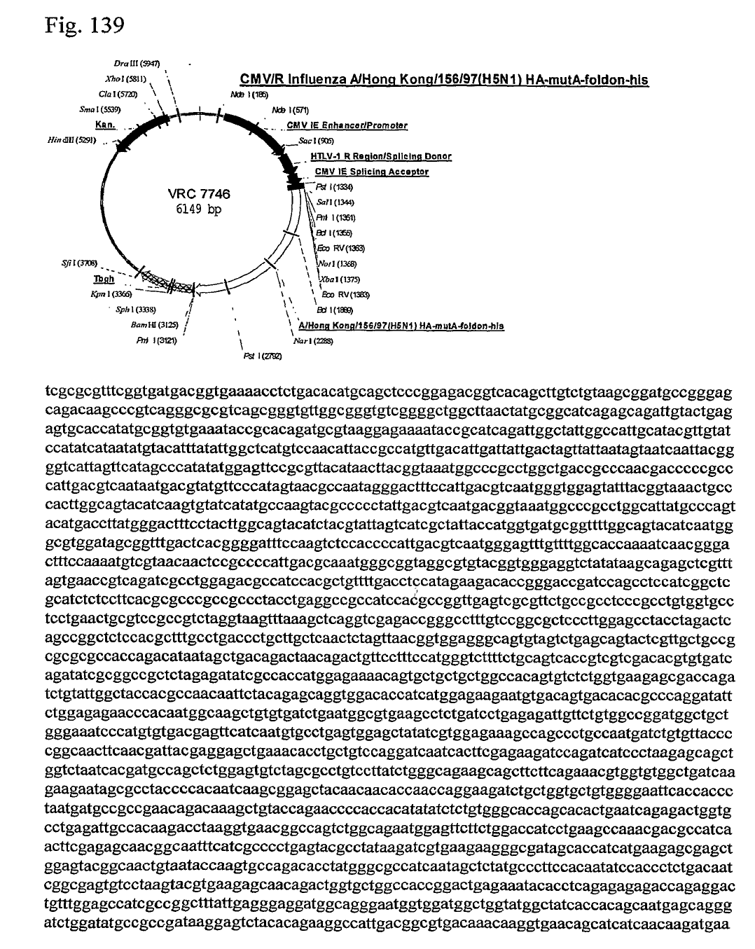

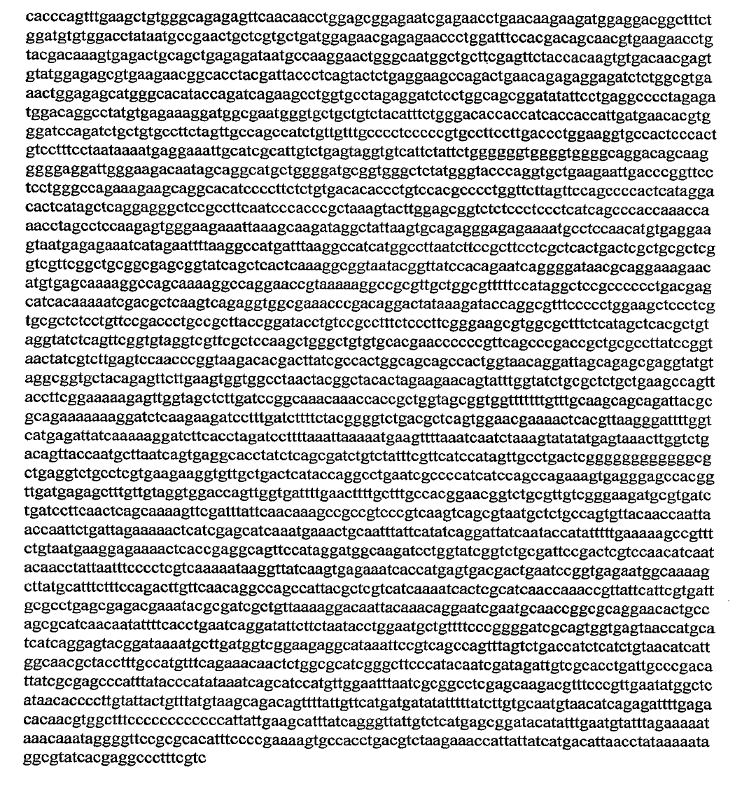

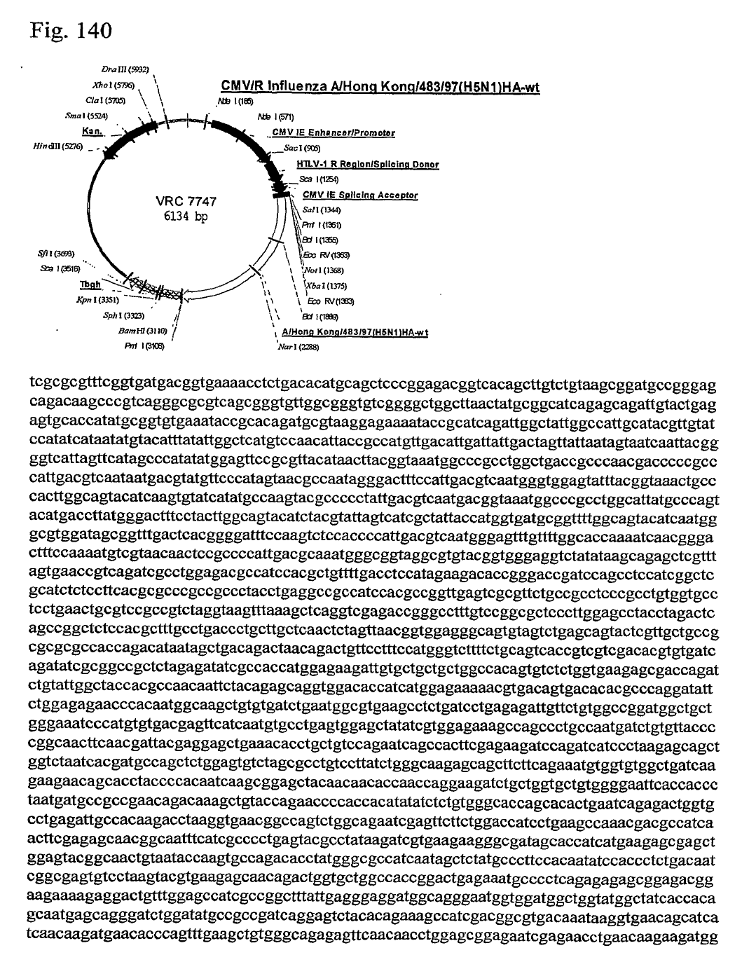

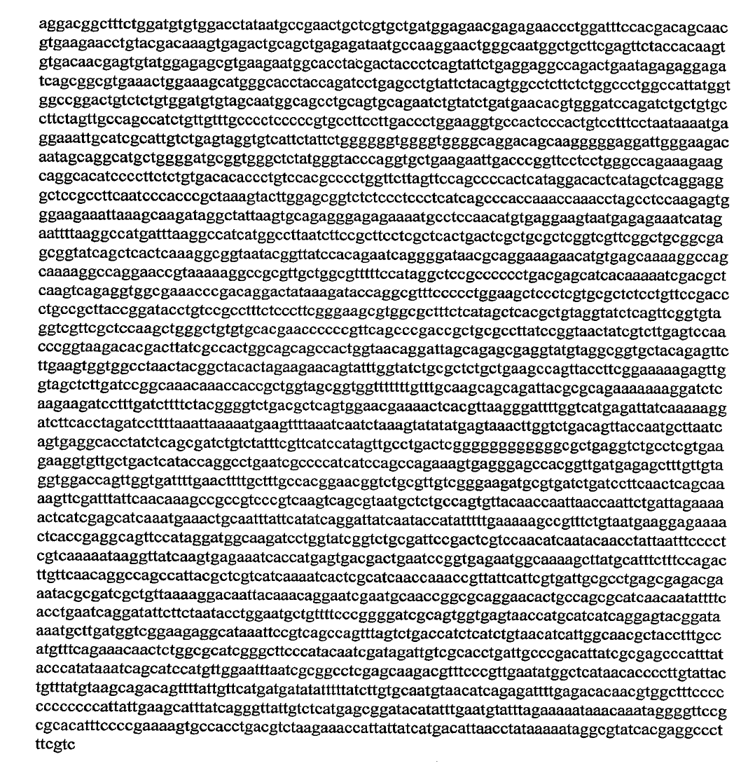

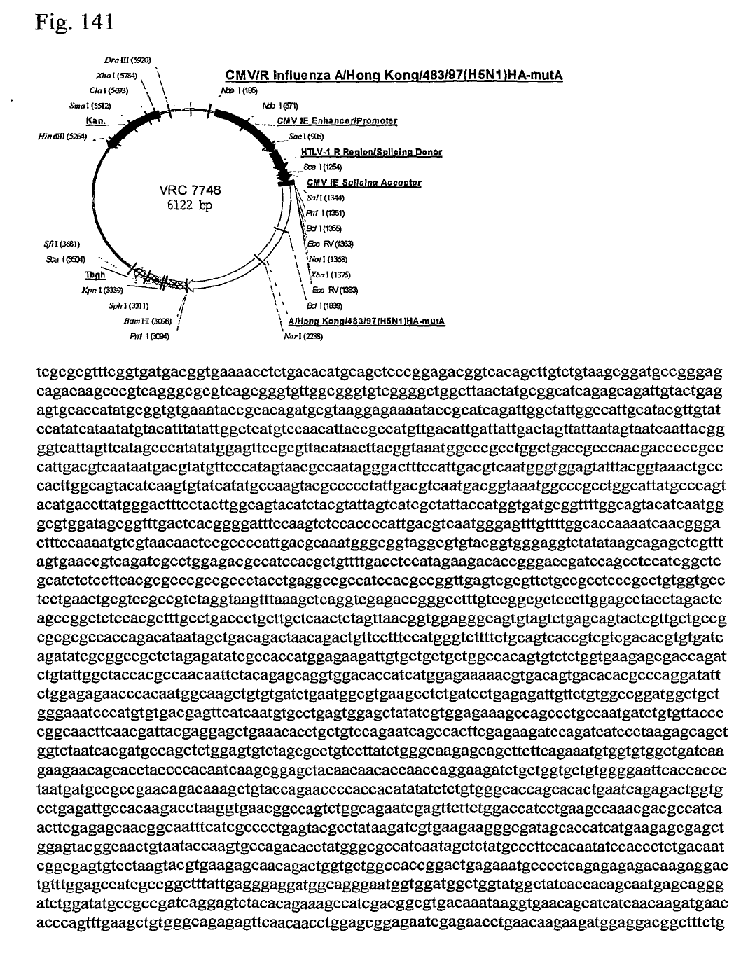

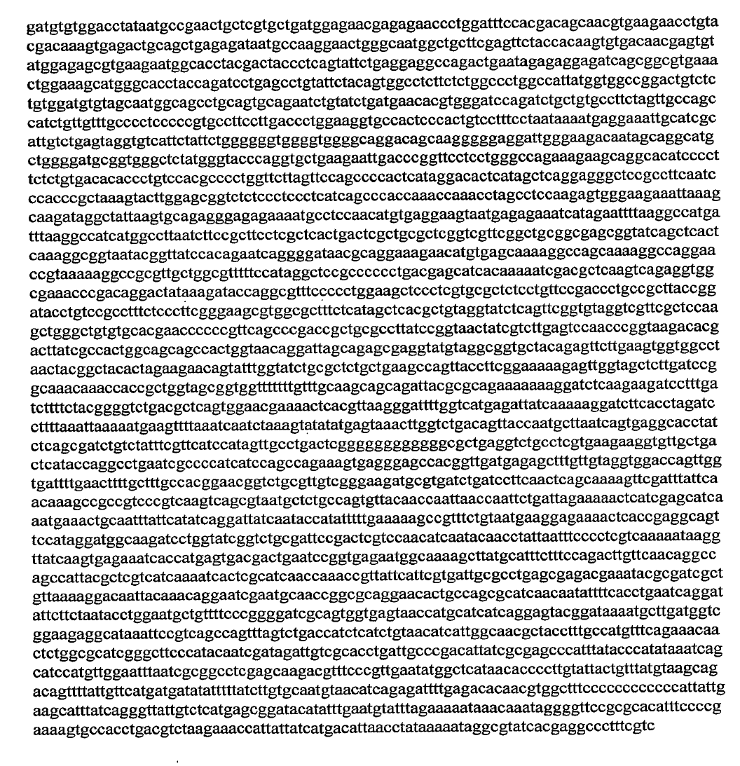

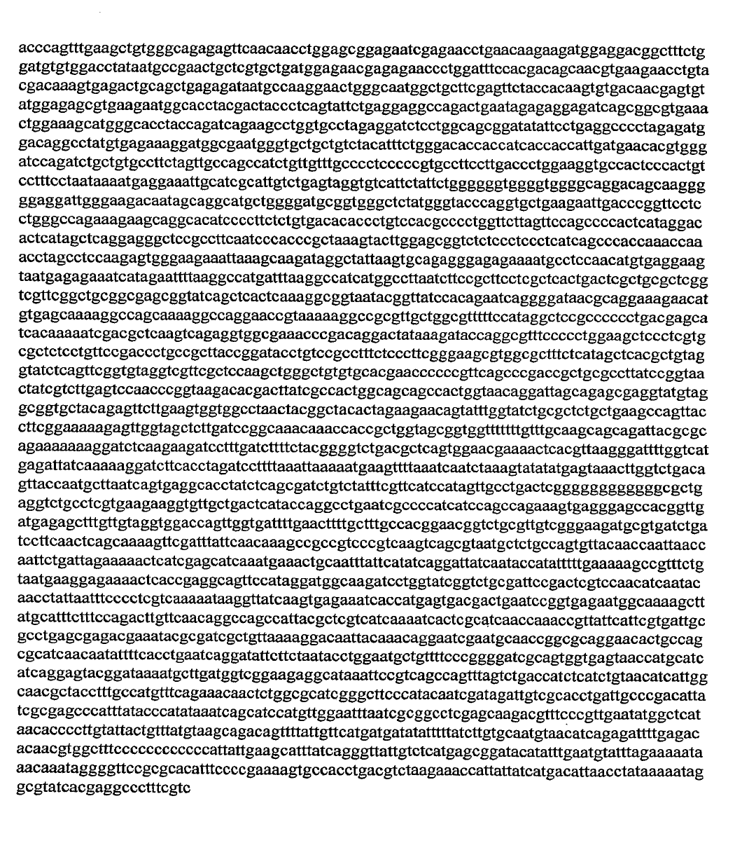

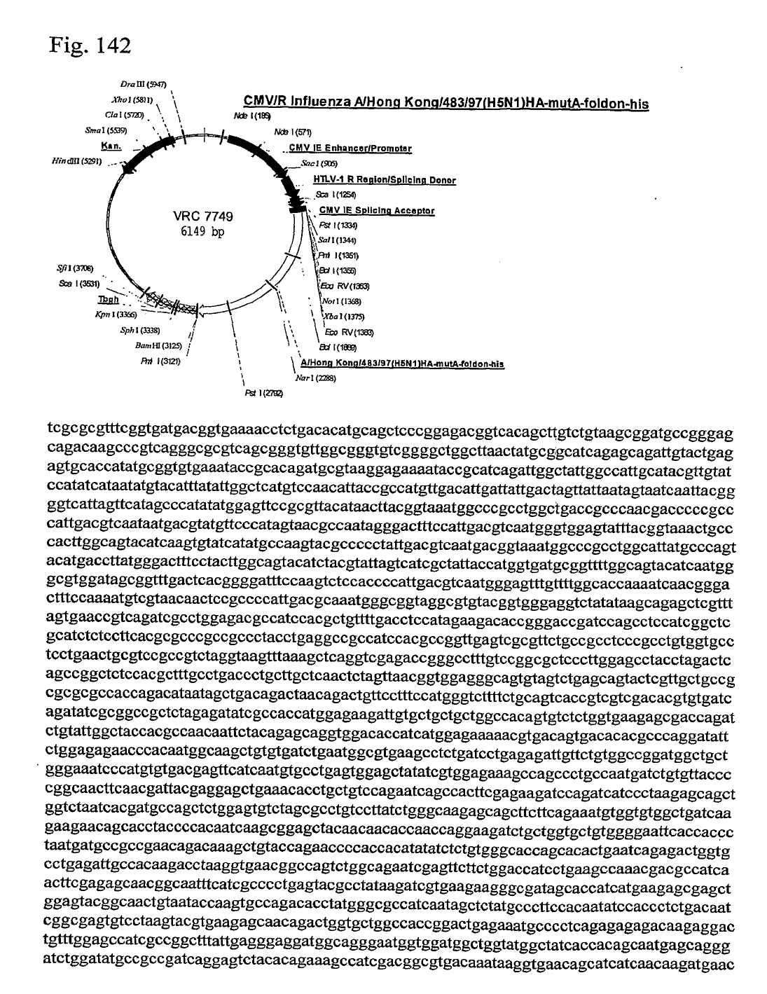

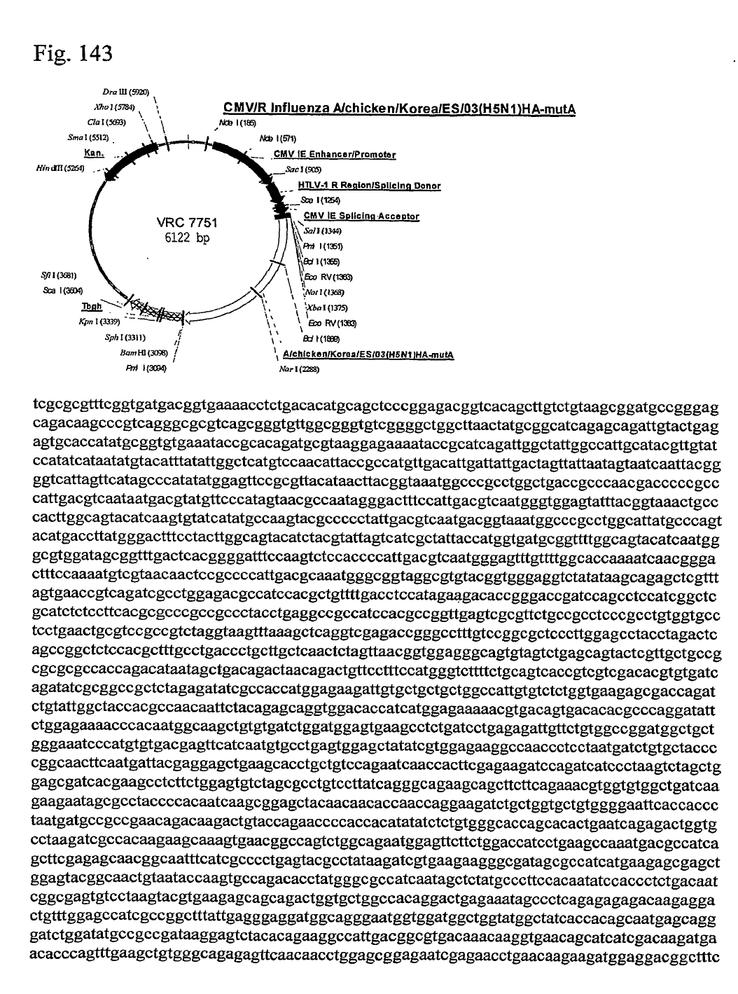

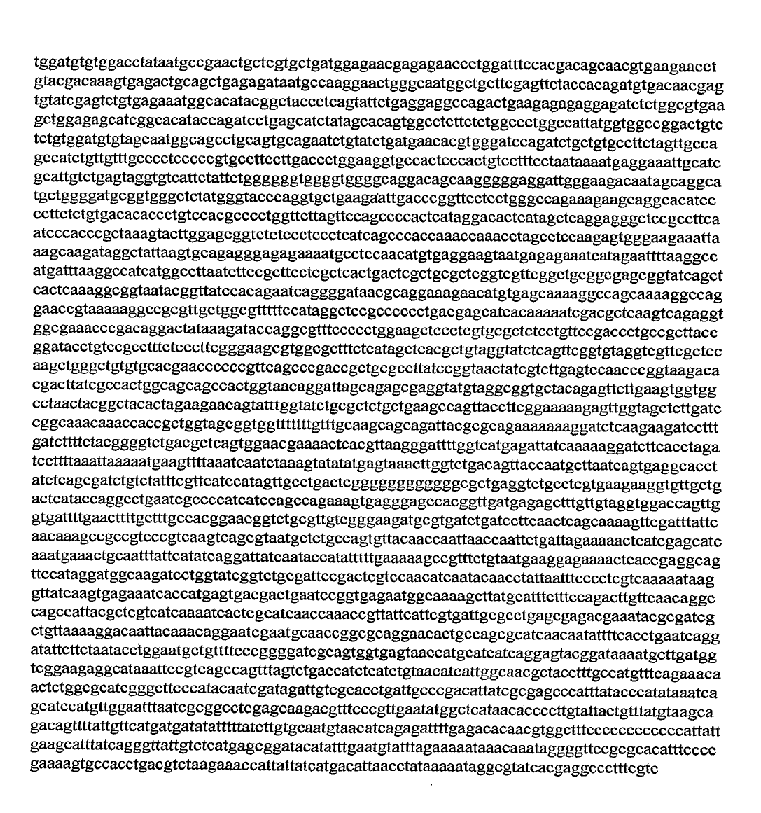

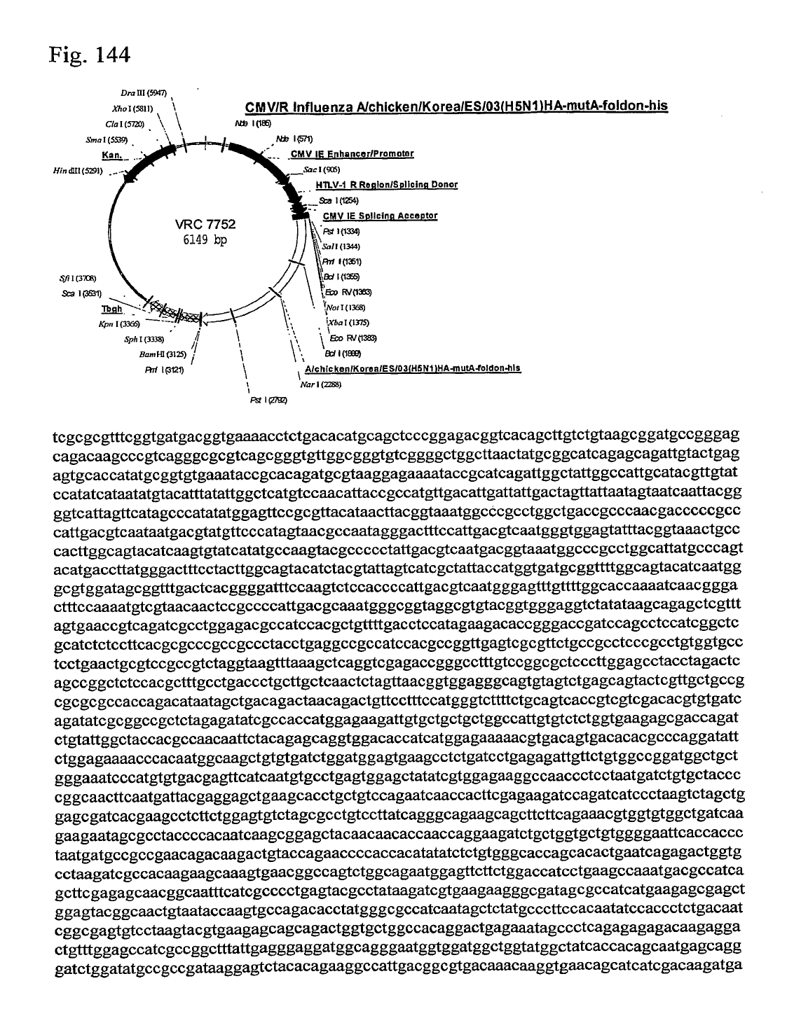

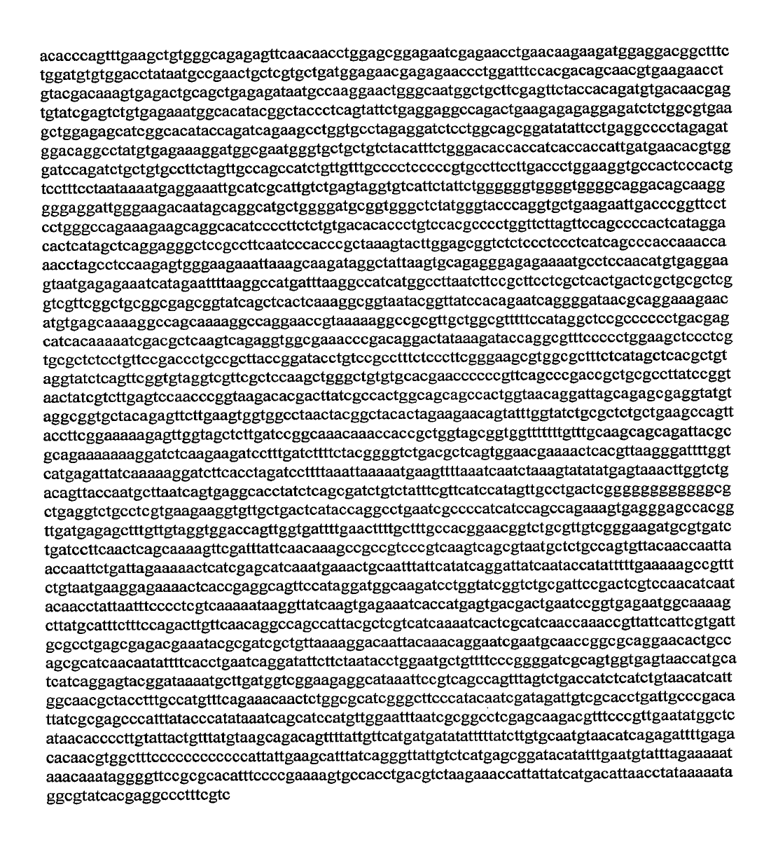

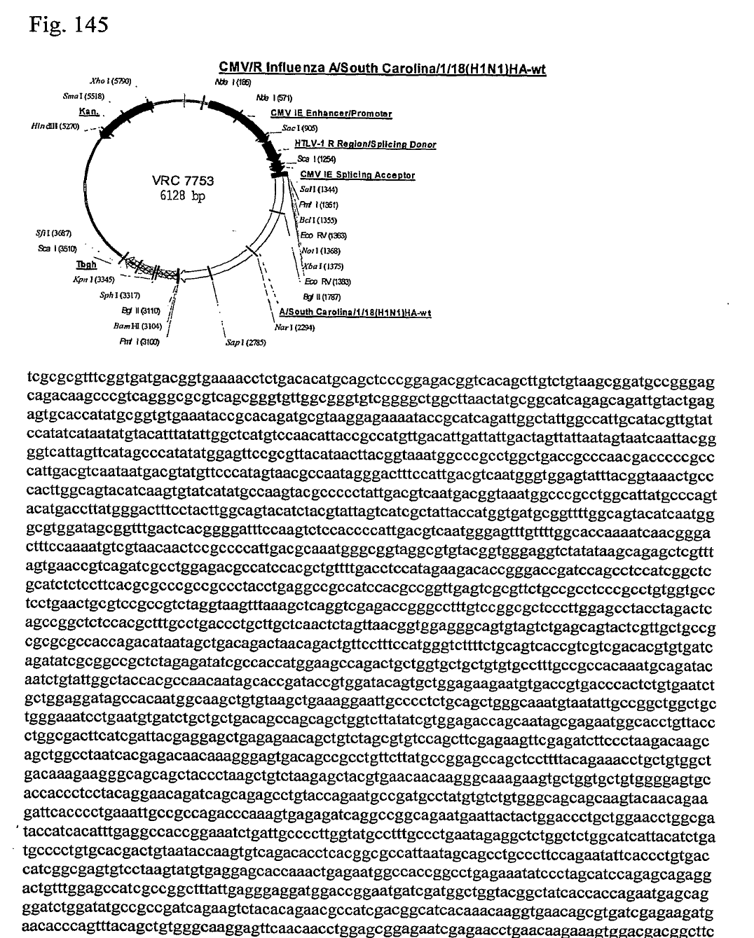

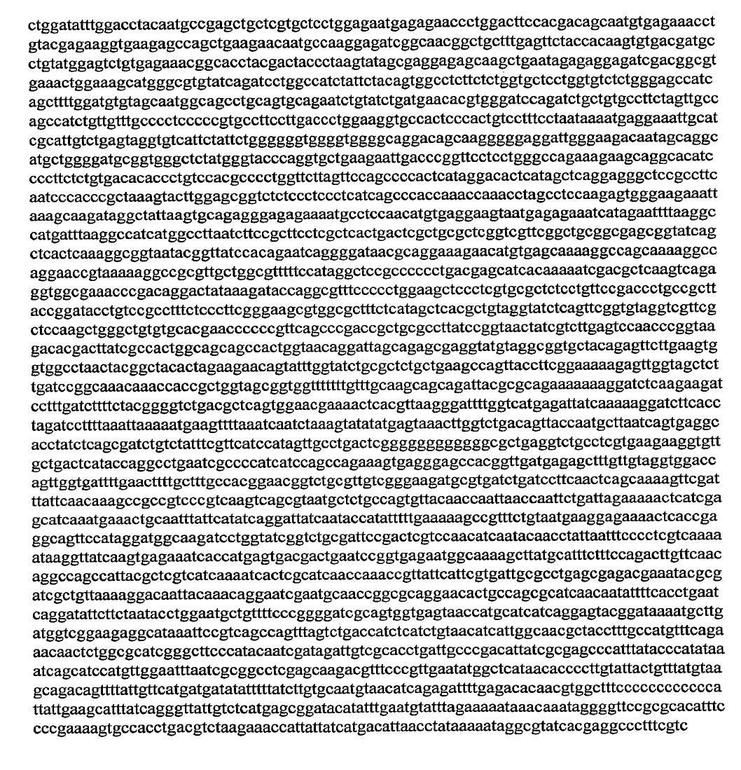

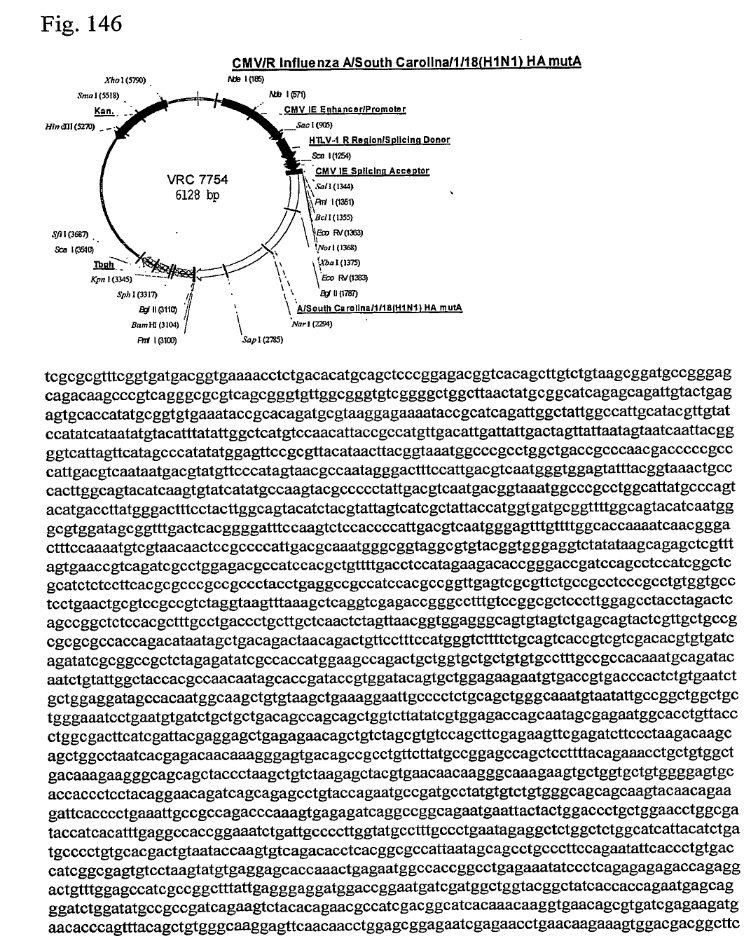

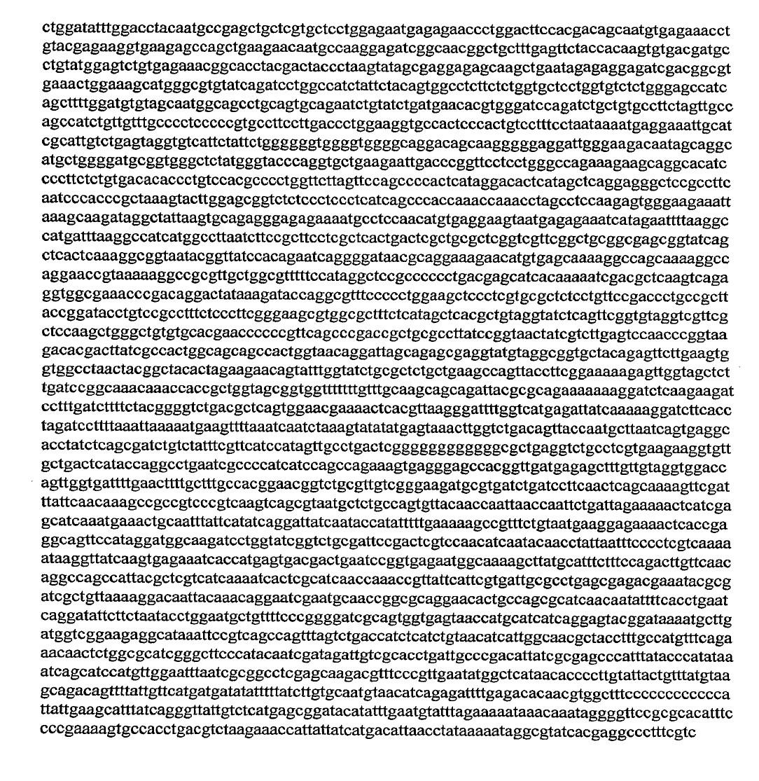

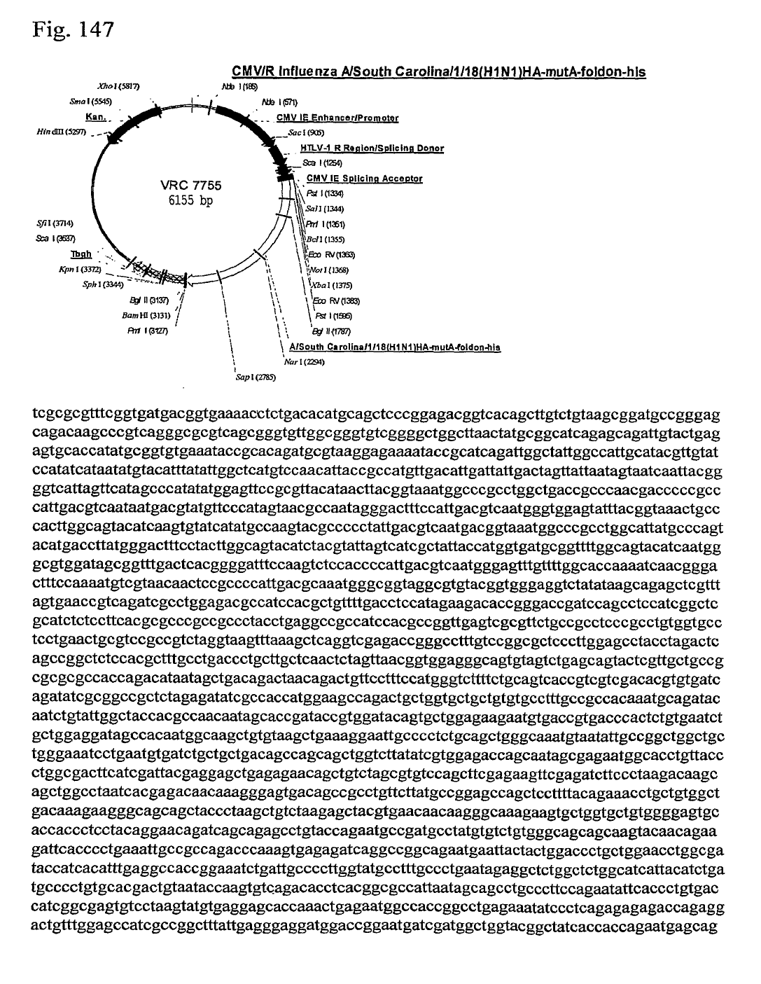

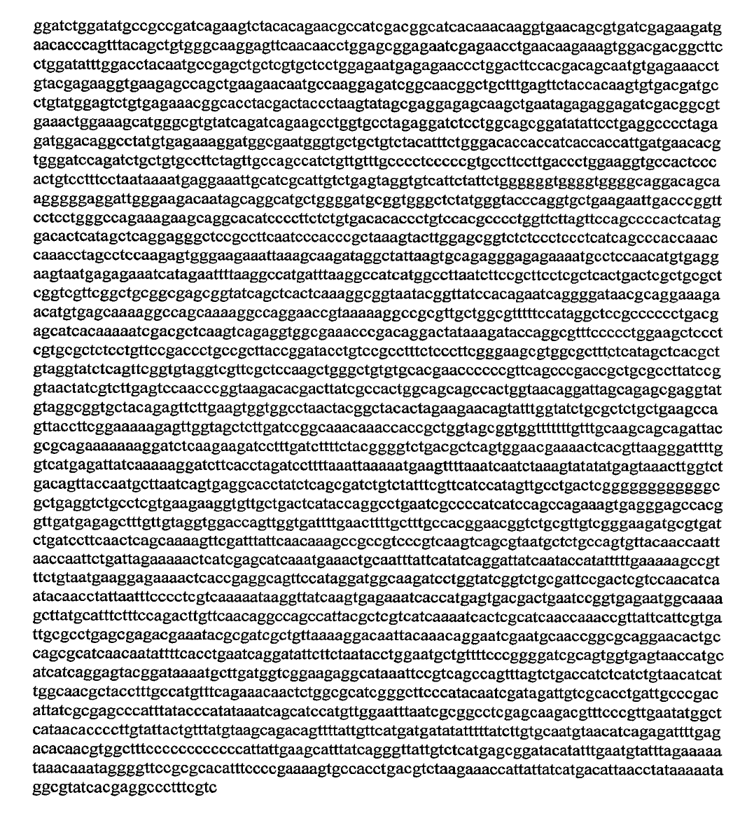

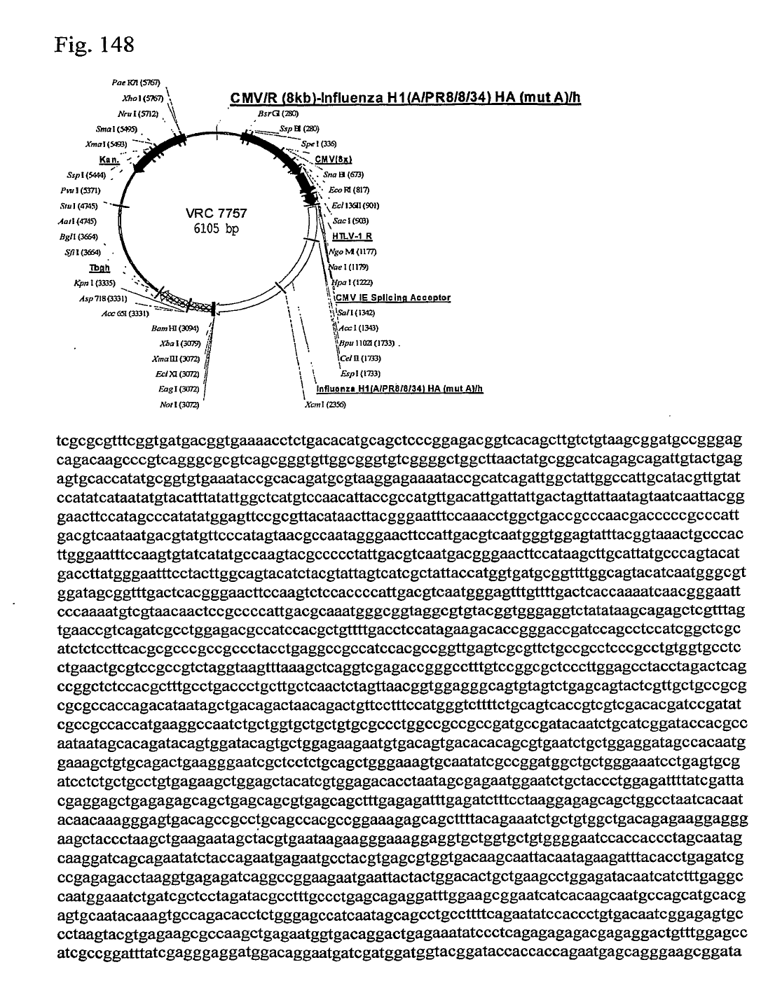

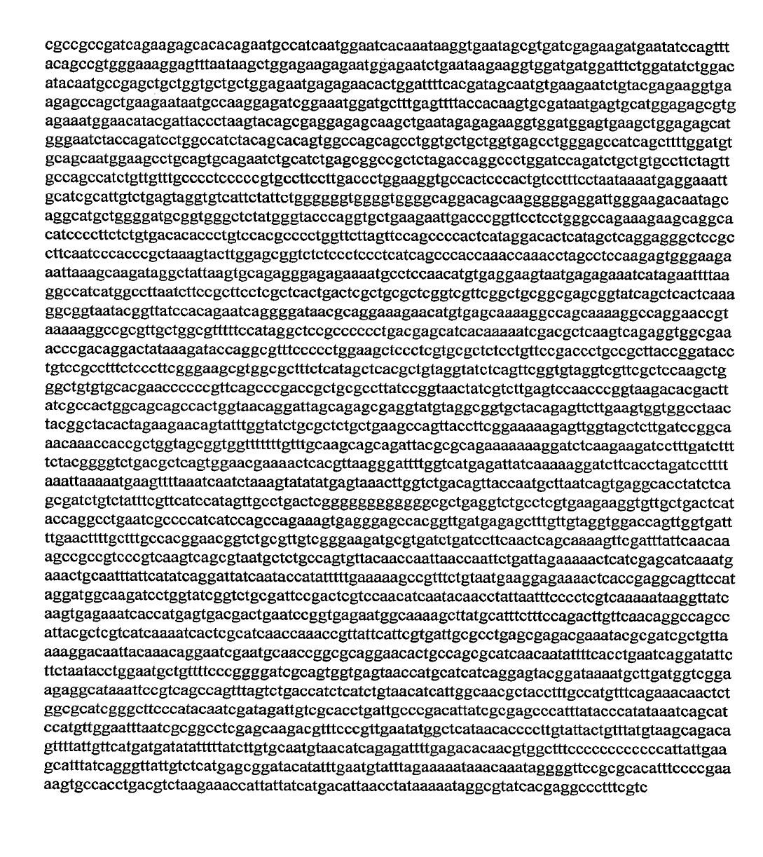

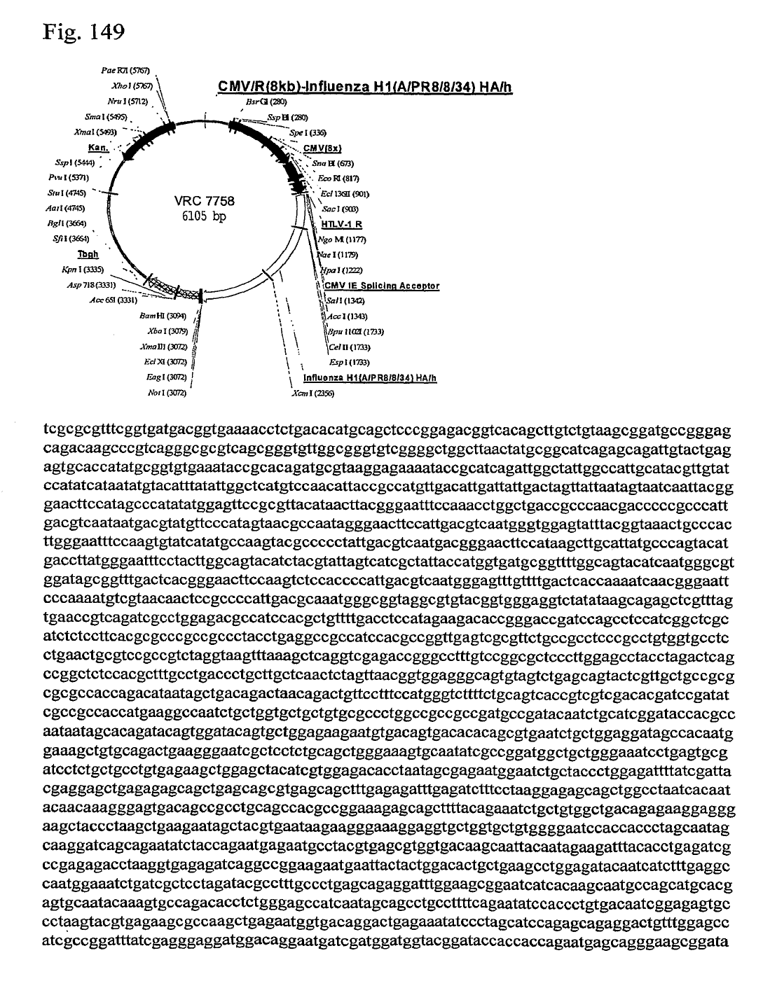

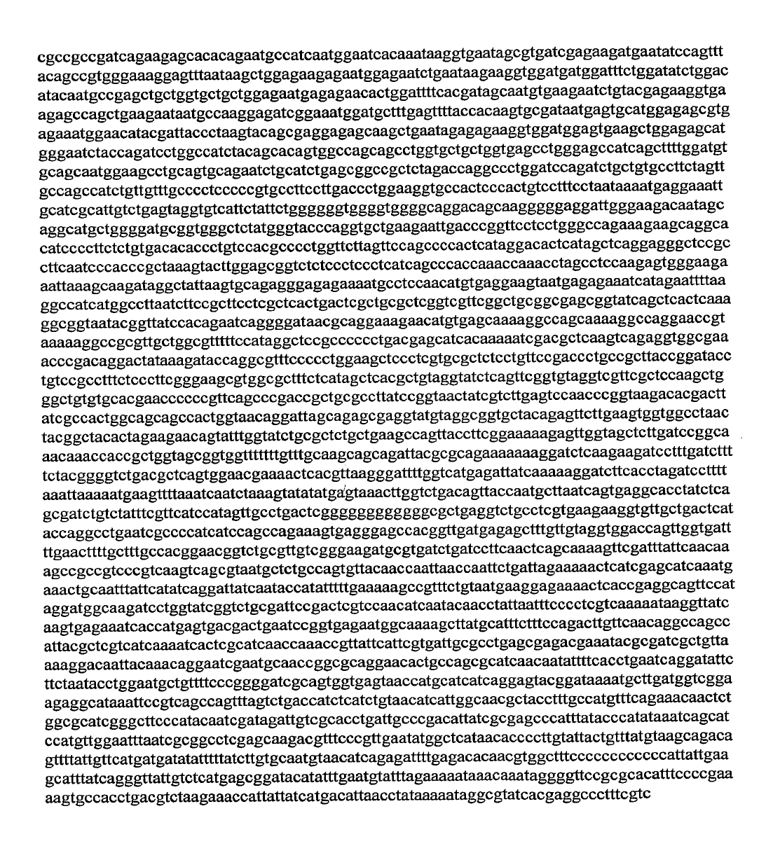

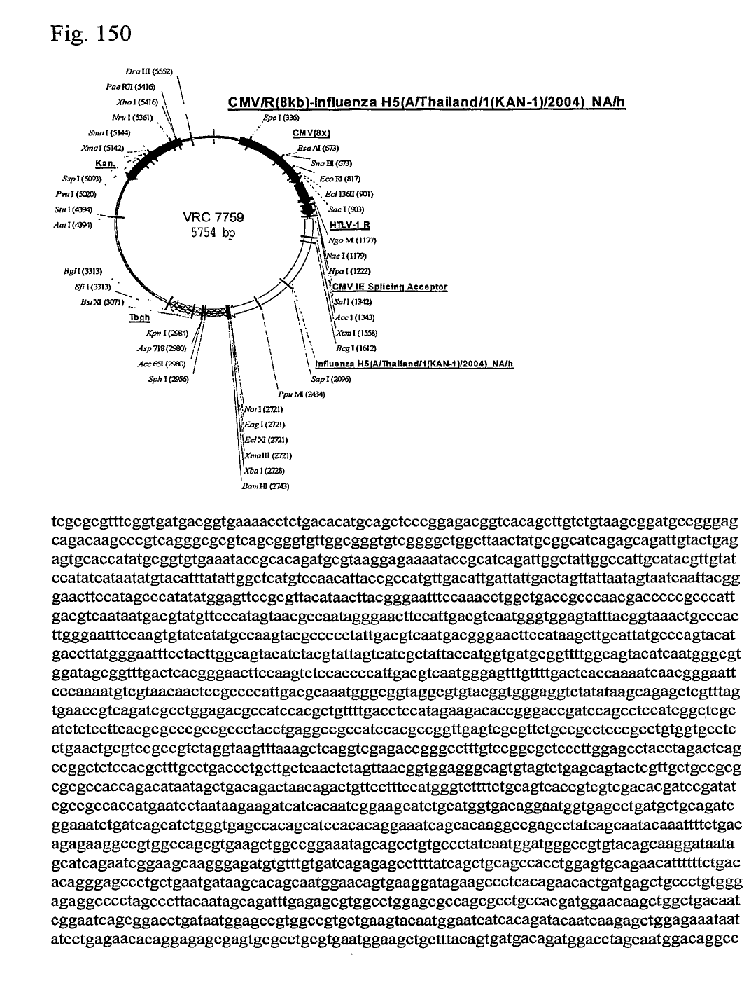

TABLE 1
Influenza HA constructs

| Construct | Construct Name/Description | SEQ ID NO | Figure |
|---|---|---|---|
| VRC 9123 | CMV/R Influenza A/Indonesia/05/05 (H5N1) HA-mut A | 1 | 1 |
| VRC 7702 | CMV/R Influenza H1(A/PR8/8/34) HA/h | 2 | 2 |
| VRC 7703 | CMV/R Influenza H1(A/PR8/8/34) HA (dPC-a)/h | 3 | 3 |
| VRC 7704 | CMV/R Influenza H1(A/PR8/8/34) HA (dPC-b)/h | 4 | 4 |
| VRC 7705 | CMV/R Influenza H5(A/Thailand/1(KAN-1)/2004) HA/h | 5 | 5 |
| VRC 7706 | CMV/R Influenza H5(A/Thailand/1(KAN-1)/2004) HA (dPC-a)/h | 6 | 6 |
| VRC 7707 | CMV/R Influenza H5(A/Thailand/1(KAN-1)/2004) HA (dPC-b)/h | 7 | 7 |
| VRC 7708 | CMV/R Influenza H5(A/Thailand/1(KAN-1)/2004) NA/h | 8 | 8 |
| VRC 7712 | CMV/R Influenza (A/PR8/8/34) M2(dTM)/h | 9 | 9 |
| VRC 7713 | CMV/R Influenza (A/PR8/8/34) TT-M2(dTM)/h | 10 | 10 |
| VRC 7714 | CMV/R Influenza (A/PR8/8/34) TT-M2/h | 11 | 11 |
| VRC 7715 | CMV/R Influenza (A/PR8/8/34) M2/h | 12 | 12 |
| VRC 7716 | CMV/R Influenza (A/Ck/Thailand/1/2004) M2(dTM)/h | 13 | 13 |
| VRC 7717 | CMV/R Influenza (A/Ck/Thailand/1/2004) TT-M2(dTM)/h | 14 | 14 |
| VRC 7718 | CMV/R Influenza (A/Ck/Thailand/1/2004) TT-M2/h | 15 | 15 |
| VRC 7719 | CMV/R Influenza (A/Ck/Thailand/1/2004) M2/h | 16 | 16 |
| 53349 | 053349pCMVR8x*/D90304-Foldon-His | 17 | 17 |
| 53350 | 053350pCMVR8x*/D90307-wt | 18 | 18 |
| 53352 | 053352pCMVR8x*/D90307-Foldon-His | 19 | 19 |
| 53353 | 053353pCMVR8x*/DQ009917-wt | 20 | 20 |
| 53355 | 053355pCMVR8x*/DQ009917-Foldon-His | 21 | 21 |
| 53356 | 053356pCMVR8x*/DQ080993-wt | 22 | 22 |
| 53358 | 053358pCMVR8x*/DQ080993-Foldon-His | 23 | 23 |
| 53359 | 053359pCMVR8x*/L43916-wt | 24 | 24 |
| 53361 | 053361pCMVR8x*/L43916-Foldon-His | 25 | 25 |
| 53362 | 053362pCMVR8x*/M21646-wt | 26 | 26 |
| 53364 | 053364pCMVR8x*/M21646-Foldon-His | 27 | 27 |
| 53365 | 053365pCMVR8x*/M35997-wt | 28 | 28 |
| 53367 | 053367pCMVR8x*/M35997-Foldon-His | 29 | 29 |
| 53320 | 053320pCMVR8x*/AAG17429-wt | 30 | 30 |
| 53322 | 053322pCMW8x*/AAG17429-Foldon-His | 31 | 31 |
| 53325 | 053325pCMVR8x*/AF028020-Foldon-His | 32 | 32 |
| 53326 | 053326pCMVR8x*/AJ404627-wt | 33 | 33 |
| 53328 | 053328pCMVR8x*/AJ404627-Foldon-His | 34 | 34 |
| 53331 | 053331pCMVR8x*/AY289929-Foldon-His | 35 | 35 |
| 53332 | 053332pCMVR8x*/AY338459-wt | 36 | 36 |
| 53334 | 053334pCMVR8x*/AY338459-Foldon-His | 37 | 37 |
| 53335 (8x) | 053335pCMVR8x*/AY531033-wt | 38 | 38 |
| 53336 (8x) | 053336pCMVR8x*/AY531033-mutant A | 39 | 39 |
| 53337 (8x) | 053337pCMVR8x*/AY531033-Foldon-His | 40 | 40 |
| 53338 | 053338pCMVR8x*/AY684886-wt | 41 | 41 |
| 53340 | 053340pCMVR8x*/AY684886-Foldon-His | 42 | 42 |
| 53955 | 053955pCMVR8x*/A/WS/33 (H1N1) HA-wt (U08904) | 43 | 43 |
| 53367 | 053367pCMVR8x*/M35997-Foldon-His | 44 | 44 |
| 53504 | 053504pAcGP67A/AY338459-Foldon-His | 45 | 45 |
| 53510 | 053510pAcGP67A/D90307-Foldon-His | 46 | 46 |
| 53515 | 053515pAcGP67A/M35997-Foldon-His 2 | 47 | 47 |
| 54567 | 054567pAcGP67A/A/Thailand/1(KAN-1)/2004 (H5N1) HA mutant A-long Foldon- | 48 | 48 |
| | His | | |
| 54568 | 054568pAcGP67A/A/Thailand/1 (KAN-1)/2004 (H5N1) HA-mutant A-short-Foldon- | 49 | 49 |
| | His | | |
| 54569 | 054569pAcGP67A/A/Thailand/1 (KAN-1)/2004 (H5N1) HA-mutant A-long-spacer- | 50 | 50 |
| | Foldon-His | | |
| 54570 | 054570pAcGP67A/A/Thailand/1 (KAN-1)/2004 (H5N1) HA-mutant A-short-spacer- | 51 | 51 |
| | Foldon-His | | |
| 53956 | 053956pCMV/R*/A/WS/33 (H1N1) HA-mutant A (U08904) | 52 | 52 |
| 53957 | 053957pCMV/R*/A/WS/33 (H1N1) HA-mutant A-Foldon-His (U08904) | 53 | 53 |
| 53967 | 053967pAcGP67A/A/WS/33 (H1N1) HA-mutant A-Foldon-His2 | 54 | 54 |
| 53329 | 053329pCMV/R*/AY289929-wt | 55 | 55 |
| 53330 | 053330pCMV/R*/AY289929-mutant A | 56 | 56 |
| 53331 | 053331pCMV/R*/AY289929-Foldon-His | 57 | 57 |
| 53503 | 053503pAcGP67A/AY289929-Foldon-His 2 | 58 | 58 |
| 51490 | 051490pPCR-Script/A/Hong Kong/156/97 (H5N1) HA-wt (AAC32088) | 59 | 59 |
| 51491 | 051491pPCR-Script/A/Hong Kong/156/97 (H5N1) HA-mutant A (AAC32088) | 60 | 60 |
| 51492 | 051492pPCR-Script/A/Hong Kong/156/97 (H5N1) HA-mutant A-Foldon-His | 61 | 61 |
| | (AAC32088) | | |
| 51493 | 051493pPCR-Script/A/Hong Kong/483/97 (H5N1) HA-wt (AAC32099.1) | 62 | 62 |
| 51494 | 051494pPCR-Script/A/Hong Kong/483/97 (H5N1) HA-mutant A (AAC32099.1) | 63 | 63 |
| 51495 | 051495pPCR-Script/A/Hong Kong/483/97 (H5N1) HA-mutant A-Foldon-His | 64 | 64 |
| | (AAC32099.1) | | |
| 51497 | 051497pPCR-Script/A/chicken/Korea/ES/03 (H5N1) HA-mutant A (AAV97603.1) | 65 | 65 |
| 51498 | 051498pPCR-Script/A/chicken/Korea/ES/03 (H5N1) HA-mutant A-Foldon-His | 66 | 66 |
| | (AAV97603.1) | | |
| 51499 | 051499pPCR-Script/Foldon-His-Tag | 67 | 67 |
| 51804 | 051804pPCR-Script/A/South Carolina/1/18 (H1N1) HA-mutant A (AF117241) | 68 | 68 |
| 51805 | 051805pPCR-Script/HA-mutant A-Foldon-His | 69 | 69 |
| 51803 | 051803pPCR-Script/HA-wt | 70 | 70 |
| 53335 (CMV/R) | 053335pCMV/R*/AY531033-wt | 71 | 71 |
| 53336 (CMV/R) | 053336pCMV/R*/AY531033-mutant A | 72 | 72 |
| 53337 (CMV/R) | 053337pCMV/R*/AY531033-Foldon-His | 73 | 73 |
| 53505 | 053505pAcGP67A/AY531033-Foldon-His 2 | 74 | 74 |
| 54508 | 054508pUC-kana/A/Thailand/1 (KAN-1)/2004 (H5N1) HA-mutant A-long-spacer- | 75 | 75 |
| | Foldon-His | | |
| 53323 | 053323pCMVR8x/AF028020-wt | 76 | 76 |
| 53344 | 053344pCMVR8x/ AY773907-wt | 77 | 77 |
| 53346 | 053346pCMVR8x/AY773907-Foldon-His | 78 | 78 |
| 53353 | 053353pCMVR8x/DQ009917-wt | 79 | 79 |
| 53355 | 053355pCMVR8x/DQ009917-Foldon-His | 80 | 80 |
| 53356 | 053356pCMVR8x/DQ080993-wt | 81 | 81 |
| 53358 | 053358pCMVR8x/DQ080993-Foldon-His | 82 | 82 |
| 53501 | 053501pAcGP67A/AF028020-Foldon-His | 83 | 83 |
| 53502 | 053502pAcGP67A/AJ404627-Foldon-His 2 | 84 | 84 |
| 53506 | 053506pAcGP67A/AY684886-Foldon-His 2 | 85 | 85 |
| 53508 | 053508pAcGP67A/AY773907-Foldon-His | 86 | 86 |
| 53511 | 053511pAcGP67A/DQ009917-Foldon-His | 87 | 87 |
| 53512 | 053512pAcGP67A/DQ080993-Foldon-His | 88 | 88 |
| 54671 | 054671pCMVR8x/AF028020-mutant A | 89 | 89 |
| 54672 | 054672pCMVR8x/AJ404627-mutant A | 90 | 90 |
| 54673 | 054673pCMVR8x/AY684886-mutant A | 91 | 91 |
| 54675 | 054675pCMVR8x/AY773907-mutant A | 92 | 92 |
| 54678 | 054678pCMVR8x/DQ009917-mutant A | 93 | 93 |
| 54679 | 054679pCMVR8x/DQ080993-mutant A | 94 | 94 |
| 53500 | 053500pAcGP67A/AAG17429-Foldon-His 2 | 95 | 95 |
| 53509 | 053509pAcGP67A/D90304-Foldon-His | 96 | 96 |
| 53513 | 053513pAcGP67A/L43916-Foldon-His | 97 | 97 |
| 53514 | 053514pAcGP67A/M21646-Foldon-His | 98 | 98 |
| 56382 | 056382pCMVR8x/A/Thailand/1 (KAN-1)/2004(H5N1) NP (AAV35112) | 99 | 99 |
| 54580 | 054580pAcGP67A/HA-mutant A-long-Foldon-His | 100 | 100 |
| 54581 | 054581pAcGP67A/HA-mutant A-short-Foldon-His | 101 | 101 |
| 54582 | 054582pAcGP67A/HA-mutant A-long-spacer-Foldon-His | 102 | 102 |
| 54583 | 054583pAcGP67A/HA-mutant A-short-spacer-Foldon-His | 103 | 103 |
| 54680 | 054680pCMVR8x*/L43916-mutant A | 104 | 104 |
| 54681 | 054681pCMVR8x*/M21646-mutant A | 105 | 105 |
| 54682 | 054682pCMVR8x*/M35997-mutant A | 106 | 106 |
| 54563 | 054563pCMVR8x*/A/Thailand/1 (KAN-1)/2004 (H5N1) HA-mutant A-long- | 107 | 107 |
| | Foldon-His | | |
| 54564 | 054564pCMVR8x*/A/Thailand/1 (KAN-1)/2004 (H5N1) HA-mutant A-short- | 108 | 108 |
| | Foldon-His | | |
| 54565 | 054565pCMVR8x*/A/Thailand/1 (KAN-1)/2004 (H5N1) HA-mutant A-long-spacer- | 109 | 109 |
| | Foldon-His | | |
| 54566 | 054566pCMVR8x*/A/Thailand/1 (KAN-1)/2004 (H5N1) HA-mutant-short-spacer- | 110 | 110 |
| | Foldon-His | | |
| 54670 | Q54670pCMVR8x*/AAG17429-mutant A | 111 | 111 |
| 54676 | 054676pCMVR8x*/D90304-mutant A | 112 | 112 |
| 54677 | 054677pCMVR8x*/D90307-mutant A | 113 | 113 |
| 53957 | 053957pCMVR8x*/A/WS/33 (H1N1) HA-mutant A-Foldon-His (U08904) | 114 | 114 |
| 54510 | 054510pCMVR8x*/A/Thailand/1 (KAN-1)/2004 (H5N1) HA-mutant A | 115 | 115 |
| 54671 | 054671pCMVR8x*/AF028020-mutant A | 116 | 116 |
| 54672 | 054672pCMVR8x*/AJ404627-mutant A | 117 | 117 |
| 54675 | 054675pCMVR8x*/AY773907-mutant A | 118 | 118 |
| 54678 | 056489pCMVR8x*/DQ009917-mutant A | 119 | 119 |
| 54679 | 054679pCMVR8x*/DQ080993-mutant A | 120 | 120 |
| 56383 | 056383pCMVR8x*/pR8(H1N1)-NPA aa (AAM75159) | 121 | 121 |
| 56384 | 056384pCMVR8x*/PR8(H1N1)-M2 aa (AAV41244) | 122 | 122 |
| 56478 | 056478pCMVR8x*/A/Brevig Mission/1/1918 (H1N1) NP (AAV48837) | 123 | 123 |
| 56479 | 056479pCMVR8x*/A/Thailand/1 (KAN-1)/2004 (H5N1) M2 (AAV35111) | 124 | 124 |
| VRC 7700 | pVR1012 INA-NP | 125 | 125 |
| VRC 7710 | pAdApt INA-NP | 126 | 126 |
| VRC 7720 | CMV/R (8κB) Influenza H5 (A/Thailand/1(KAN-1)/2004) HA/h | 127 | 127 |
| VRC 7730 | CMV/R 8κB Influenza A/South Carolina/1/18(H1N1) HA-wt | 128 | 128 |
| VRC 7731 | CMV/R 8κB Influenza A/South Carolina/1/18 (H1N1) HA-mut A | 129 | 129 |
| VRC 7732 | CMV/R 8κB Influenza A/South Carolina/1/18 (H1N1) HA mut A-long-Foldon-His | 130 | 130 |
| VRC 7733 | CMV/R 8κB Influenza A/South Carolina/1/18 (H1N1) HA mut A-short-Foldon-His | 131 | 131 |
| VRC 7734 | CMV/R 8κB Influenza A/South Carolina/1/18 (H1N1) HA mut A long-spacer- | 132 | 132 |
| | Foldon-His | | |
| VRC 7735 | CMV/R 8κB Influenza A/South Carolina/1/18 (H1N1) HA mut A short-spacer- | 133 | 133 |
| | Foldon-His | | |
| VRC 7742 | CMVR HI(A/PR8/8/34) HA mutA-short-Foldon-His | 134 | 134 |
| VRC 7721 | CMV/R 8κB Influenza H5 (A/Thailand/1(KAN-1)/2004) HA mut A/h | 135 | 135 |
| VRC 7743 | CMVR HI(A/PR8/8/34) HA mut A-long-Foldon-His | 136 | 136 |
| VRC 7744 | CMV/R Influenza A/Hong Kong/156/97 (H5N1) HA-wt | 137 | 137 |
| VRC 7745 | CMV/R Influenza A/Hong Kong/156/97 (H5N1) HA-mut A | 138 | 138 |
| VRC 7746 | CMV/R Influenza A/Hong Kong/156/97 (H5N1) HA-mut A-Foldon-His | 139 | 139 |
| VRC 7747 | CMV/R Influenza A/Hong Kong/483/97 (H5N1) HA-wt | 140 | 140 |
| VRC 7748 | CMV/R Influenza A/Hong Kong/483/97 (H5N1) HA-mut A | 141 | 141 |
| VRC 7749 | CMV/R Influenza A/Hong Kong/483/97 (H5N1) HA-mut A-Foldon-His | 142 | 142 |
| VRC 7751 | CMV/R Influenza A/chicken/Korea/ES/03(H5N1) HA-mut A | 143 | 143 |
| VRC 7752 | CMV/R Influenza A/chicken/Korea/ES/03(H5N1) HA-mut A-Foldon-His | 144 | 144 |
| VRC 7753 | CMV/R Influenza A/South Carolina/1/18 (H1N1) HA-wt | 145 | 145 |
| VRC 7754 | CMV/R Influenza A/South Carolina/1/18 (H1N1) HA-mut A | 146 | 146 |
| VRC 7755 | CMV/R Influenza A/South Carolina/1/18 (H1N1) HA-mut A-Foldon-His | 147 | 147 |
| VRC 7757 | CMV/R (8κB)-Influenza H1(A/PR8/8/34) HA (mut A)/h | 148 | 148 |
| VRC 7758 | CMV/R (8κB)-Influenza H1(A/PR8/8/34) HA/h | 149 | 149 |
| VRC 7759 | CMV/R (8κB)-Influenza H5 (A/Thailand/1(KAN-1)/2004) NA/h | 150 | 150 |

### DETAILED DESCRIPTION OF THE PREFERRED EMBODIMENT

### Part 1

### Influenza A

Influenza A is an enveloped negative single-stranded RNA virus that infects a wide range of avian and mammalian species. The influenza A viruses are classified into serologically-defined antigenic subtypes of the hemagglutinin (HA) and neuraminidase (NA) major surface glycoproteins (WHO Memorandum 1980 Bull WHO 58:585-591). The nomenclature meets the requirement for a simple system that can be used by all countries and it has been in effect since 1980. It is based on data derived from double immunodiffusion (DID) reactions involving hemagglutinin and neuraminidase antigens.

Double immunodiffusion (DID) tests are performed as described previously (Schild, GC et al. 1980 Arch Virol 63:171-184). Briefly, tests are carried out in agarose gels (HGT agarose, 1% phosphate-buffered saline, pH 7.2 containing 0.01 percent sodium azide). Preparations of purified virus particles containing 5-15 mg virus protein per ml (or an HA titer with chick erythrocytes of 10^{5.5}-10^{6.5} hemagglutinin units per 0.25 ml) are added in 5-10 μl volumes to wells in the gel. The virus particles are disrupted in the wells by the addition of sarcosyl detergent NL97, 1 percent final concentration). The precipitin reactions are either photographed without staining or, the gels are dried and stained with Coomassie Brilliant Blue.

The DID test, when performed using hyperimmune sera specific to one or other of the antigens, provides a valuable method for comparing antigenic relationships. Similarities between antigens are detected as lines of common precipitin, whereas the existence of variation between antigens is revealed by spurs of precipitin when different antigens are permitted to diffuse radically inwards toward a single serum. Based on the results of DID tests on influenza A viruses from all species, the H antigens can be grouped into 16 subtypes as indicated in Table 2).

TABLE 2
Hemagglutinin subtypes of influenza A viruses
isolated from humans, lower mammals and birds

| Sub- | Species of origin^{a} | | | |
|---|---|---|---|---|
| types | Humans | Swine | Horses | Birds |
| H1^{b} | PR/8/34 | Sw/Ia/15/30 | — | Dk/Alb/35/76 |
| H2 | Sing/1/57 | — | — | Dk/Ger/1215/73 |
| H3 | HK/1/68 | Sw/Taiwan/70 | Eq/Miami/1/63 | Dk/Ukr/1/63 |
| H4 | — | — | — | Dk/Cz/56 |
| H5 | — | — | — | Tern/S.A./61 |
| H6 | — | — | — | Ty/Mass/3740/65 |
| H7 | — | — | Eq/Prague/1/56 | FPV/Dutch/27 |
| H8 | — | — | — | Ty/Ont/6118/68 |
| H9 | — | — | — | Ty/Wis/1/66 |
| H10 | — | — | — | Ck/Ger/N/49 |
| H11 | — | — | — | Dk/Eng/56 |
| H12 | — | — | — | Dk/Alb/60/76 |
| H13 | — | — | — | Gull/MD/704/77 |
| H14 | — | — | — | Dk/Gurjev/263/82 |
| H15 | — | — | — | Dk/Austral/3431/83 |
| H16 | — | — | — | A/Black-headed |
| | | | | Gull/Sweden/5/99 |

^{a}The reference strains of influenza viruses, or the first isolates from that species, are presented.
^{b}Current subtype designation. From WHO Memorandum 1980 Bull WHO 58: 585-591.

The influenza A genome consists of eight single-stranded negative-sense RNA molecules (FIG. 151). Three types of integral membrane protein-hemagglutinin (HA), neuraminidase (NA), and small amounts of the M2 ion channel protein-are inserted through the lipid bilayer of the viral membrane. The virion matrix protein M1 is thought to underlie the lipid bilayer but also to interact with the helical ribonucleoproteins (RNPs). Within the envelope are eight segments of single-stranded genome RNA (ranging from 2341 to 890 nucleotides) contained in the form of an RNP. Associated with the RNPs are small amounts of the transcriptase complex, consisting of the proteins PB1, PB2, and PA. The coding assignments of the eight RNA segments are also illustrated in FIG. 151.

### Antigenic Shift and Drift

The segmentation of the influenza A genome facilitates reassortment among strains, when two or more strains infect the same cell. Reassortment can yield major genetic changes, referred to as antigenic shifts. In contrast, antigenic drift is the accumulation of viral strains with minor genetic changes, mainly amino acid substitutions in the HA and NA proteins. Influenza A nucleic acid replication by the virus-encoded RNA-dependent RNA polymerase complex is relatively error-prone, and these point mutations (˜1/10⁴ bases per replication cycle) in the RNA genome are the major source of genetic variation for antigenic drift.

Selection favors human influenza A strains with antigenic drift and shift involving the HA and NA proteins because these strains are able to evade neutralizing antibody from prior infection or vaccination. This selection allows viral reinfection with a new subtype (shift) or the same viral subtype (drift). Antigenic shifts caused three of the major influenza A pandemics in the twentieth century, including the 1918 H1N1 (Spanish flu), the 1957 H2N2 (Asian flu) and the 1968 H3N2 (Hong Kong flu) outbreaks. Antigenic drift accounts for the annual nature of flu epidemics. It also explains the reduced efficacy of influenza A vaccination, which is based on neutralizing antibody: For a particular subtype, if the amino acid sequence of the HA protein used in vaccination does not match that encountered during the epidemic, antibody neutralization may be ineffective.

### Hemagglutinin A

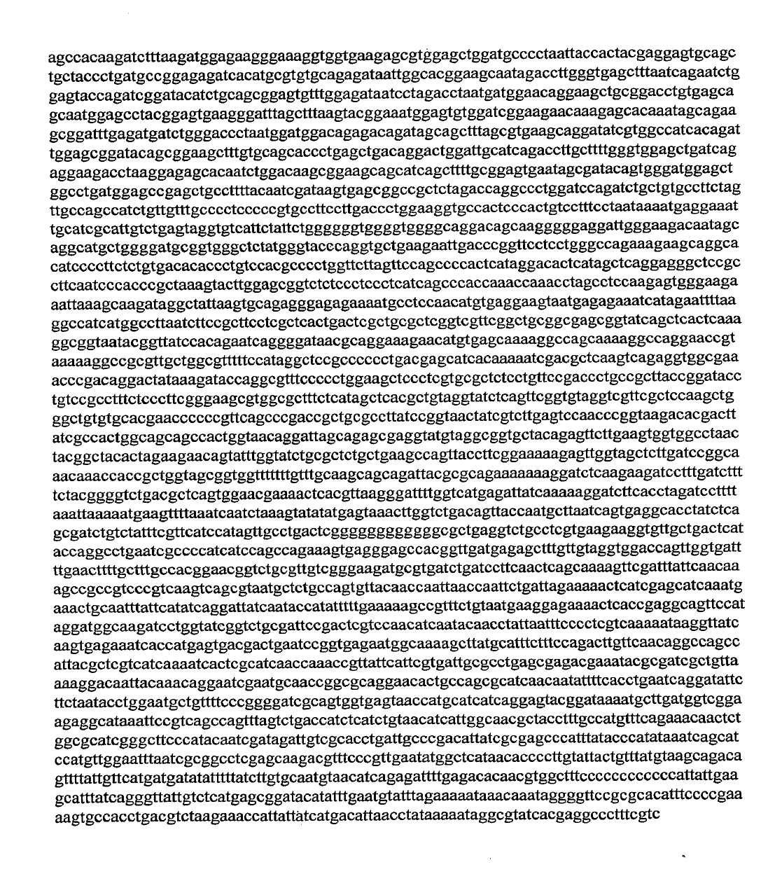

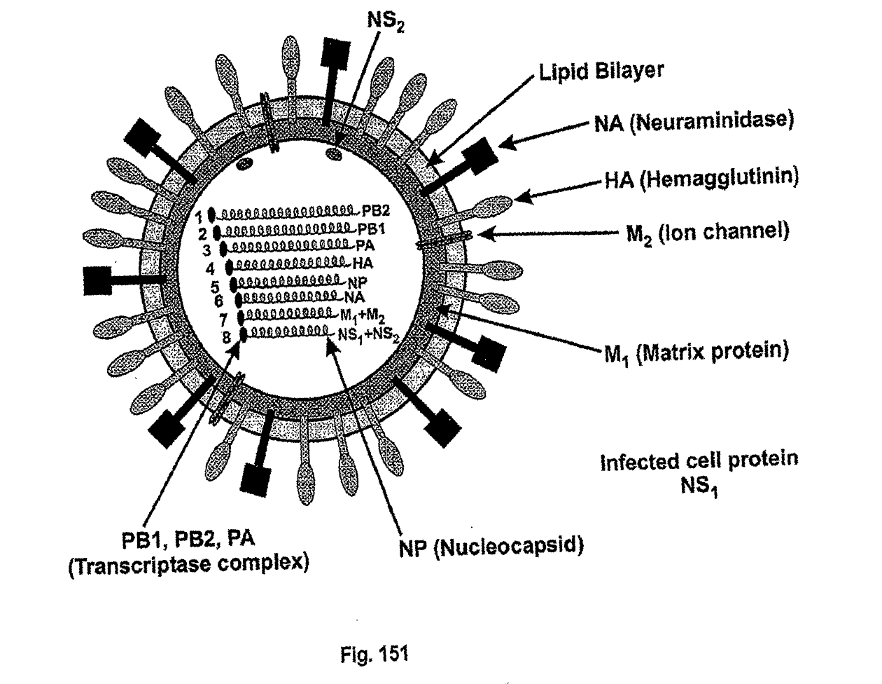

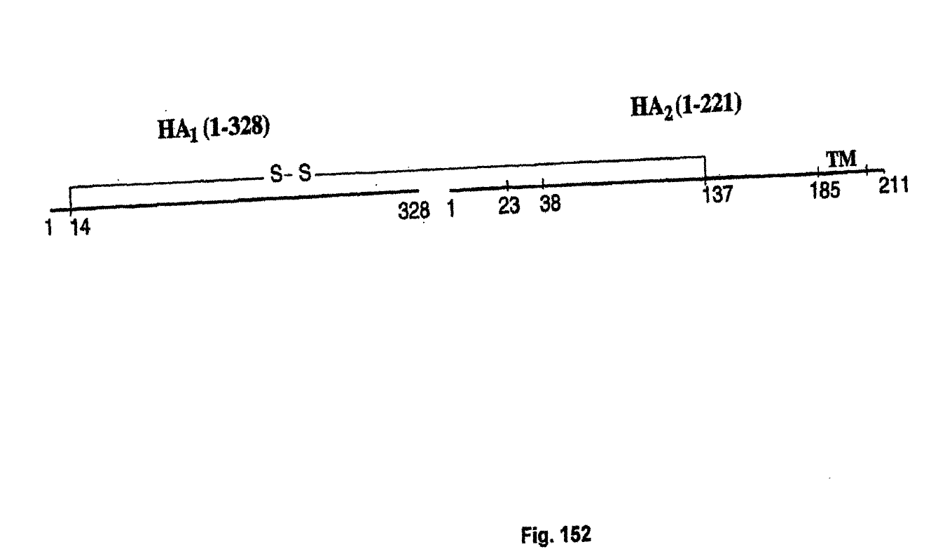

HA is encoded on a separate RNA molecule. HA is involved in viral attachment to terminal sialic acid residues on host cell glycoproteins and glycolipids. After viral entry into an acidic endosomal compartment of the cell, HA is also involved in fusion with the cell membrane, which results in the intracellular release of the virion contents. HA is synthesized as an HA₀ precursor that forms noncovalently bound homotrimers on the viral surface. The HA₀ precursor is cleaved by host proteases at a conserved arginine residue to creat two subunits, HA₁ and HA₂, which are associated by a single disulfide bond (FIG. 152). This cleavage event is required for productive infection.

HA is a critical determinant of the pathogenicity of avian influenza viruses, with a clear link between HA cleavability and virulence. The HA proteins of highly pathogenic H5 and H7 viruses contain multiple basic amino acid residues at the cleavage sites which are recognized by ubiquitous proteases, furin and PC6. For this reason, these viruses can cause systemic infections in poultry. Two groups of proteases are responsible for HA cleavage. The first group recognizes a single arginine and cleaves all HAs. Members of this group include plasmin, blood-clotting factor X-like proteases, tryptase Clara, miniplasmin, and bacterial proteases. The second group of proteases that cleaves HA proteins comprises the ubiquitous intracellular subtilisin-related endoproteases furin and PC6. These enzymes are calcium dependent, have an acidic pH optimum, and are located in the Golgi and/or trans-Golgi network.

The mature HA forms homotrimers. The crystallographic study of HA revealed the major features of the trimer structure: (a) a long fibrous stem that is comprised of a triple-stranded coiled coil of α-helices derived from the three HA2 parts of the molecule, and (b) the globular head, which is also comprised of three identical domains whose sequences are derived from the HA1 portions of the three monomers.

### Oligomerization Motifs

Several exogenous oligomerization motifs have been successfully used to promote stable trimers of soluble recombinant proteins: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin ‘foldon’ (Miroshnikov et al. 1998 Protein Eng 11:329-414). The fibritin foldon, a 27 amino acid sequence (GYIPEAPRDGQAYVRKDGEWVLLSTF, SEQ ID NO: 155), adopts a β-propeller conformation, and can fold and trimerize in an autonomous way (Tao et al. 1997 Structure 5:789-798). It has been reported recently that this foldon can successfully induce stable trimerization of other fibrous motifs such as phage T4 short-tail fibers and adenovirus fibers, as well as viral human immunodeficiency virus glycoprotein gp140.

### Nucleoprotein (NP)

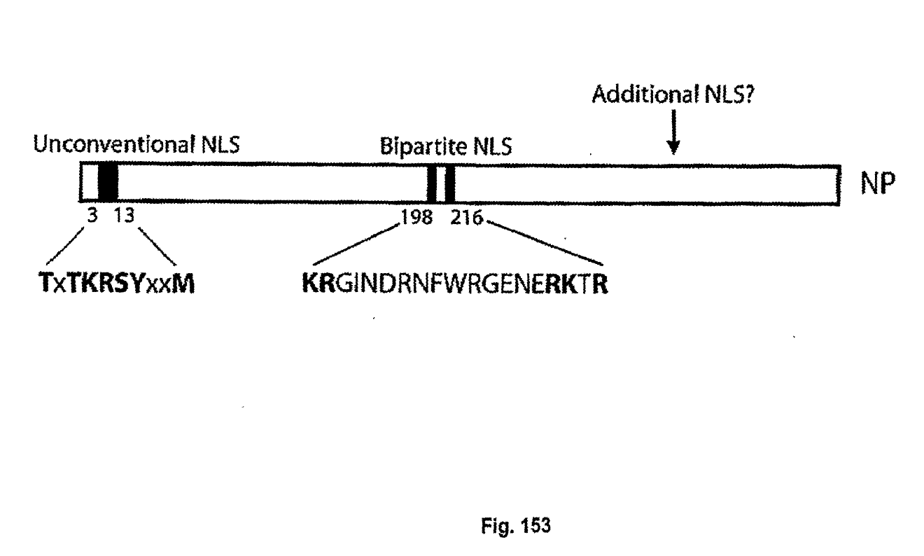

The major viral protein in the ribonucleoprotein complex is the NP, which coats the RNA. A schematic representation of the influenza A NP is shown in FIG. 153. The relative positions of the nuclear localization signals (NLS) are indicated, and the amino acids critical for activity are shown in bold type. Additional NLS have been postulated. Investigators proposed that an NLS is located between amino acids 320 and 400 and that NP may contain a conformational NLS.

### Neuraminidase

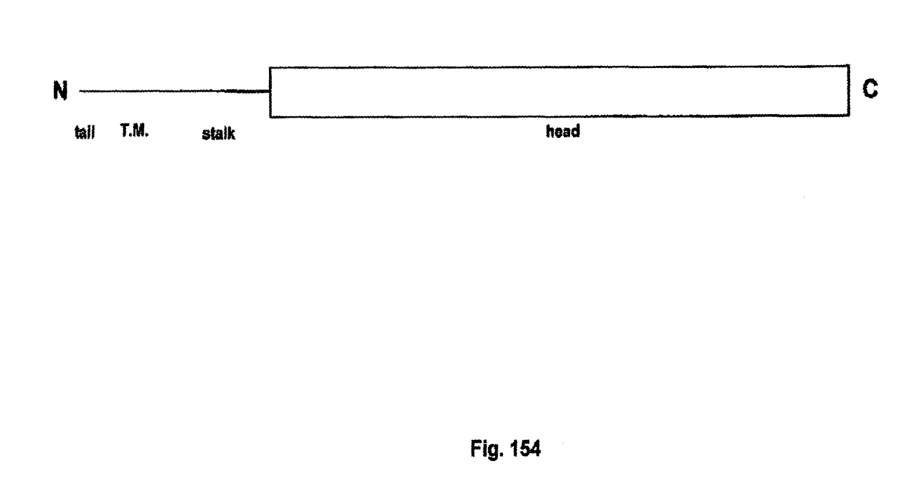

NA is encoded on a separate RNA molecule. A schematic representation of the influenza A NA protein is shown in FIG. 154. NA cleaves terminal sialic acid residues of influenza A cellular receptors and is involved in the release and spread of mature virions. It may also contribute to initial viral entry. NA is the target of inhibitor drugs such as oseltamivir and zanamivir.

### M2 Protein

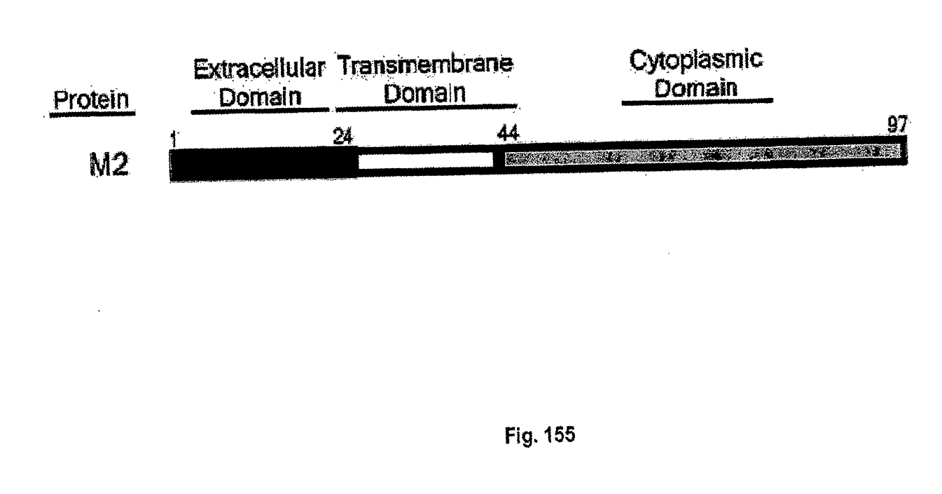

A single RNA segment encodes two matrix proteins, M1 and M2, which are generated by mRNA splicing. M1 is entirely internal and located immediately below the lipid bilayer of the virus. M2 serves as an ion channel that has a small extracellular surface domain. A schematic representation of the influenza A M2 protein is shown in FIG. 155. M2 is the target of the antiviral drugs amantidine and rimantidine.

### Types of Modifications

Described herein are modified influenza HA proteins that improve the immune response to native HA and expose the core protein for optimal antigen presentation and recognition. Weissenhom et al., 1998 Molecular Cell 2:605-616 proposes a core protein as a model for a fusion intermediate of viral glycoproteins, where the glycoproteins are characterized by a central triple stranded coiled coil followed by a disulfide-bonded loop that reverses the chain direction and connects to an α helix packed antiparallel to the core helices, as, for example, in the case of Ebola Zaire GP2, Murine Moloney Leukemia virus (MuMoLv) 55-residue segment of the TM subunit (Mo-55), low-pH-treated influenza HA2, protease resistant core of HIV gp41, and SIV gp41. Thus, the strategy for improving the immune response by exposing the protease resistant core embraces HA2 as a viral membrane fusion protein that is characterized by a central triple stranded coiled coil followed by a disulfide-bonded loop that reverses the chain direction and connects to an α helix packed antiparallel to the core helices.

To develop influenza variants that might effectively induce humoral and cellular immunity, a series of plasmid expression vectors were generated. Influenza proteins are encoded by nucleic acid sequences that contain RNA structures that may limit gene expression. These vectors were therefore synthesized using codons found in human genes that allow these structures to be eliminated without affecting the amino acid sequence.

To alter HA immunogenicity, an internal deletion was designed to stabilize and expose functional domains of the protein that might be present in an extended helical structure prior to the formation of the six-member coiled-coil structure in the hairpin intermediate (Weissenhom W et al. 1997 Nature 387:426-430). To generate this putative pre-hairpin structure, the cleavage site was removed to prevent the proteolytic processing of HA and stabilize the protein by linking HA1 covalently to HA2. These deletions were introduced into full-length and COOH-terminal truncation mutants.

The ability of these influenza proteins to elicit an immune response was determined in mice by injection with these plasmid DNA expression vectors. Antibody responses were monitored by the microneutralization assay and viral pseudotype assay.

To determine whether these modifications adversely affected CTL responses, vaccinated animals were tested for an increase in antigen-specific CD4 and CD8 T cells, as determined by intracellular cytokine staining to measure cells synthesizing either IFN-γ or TNF-α.

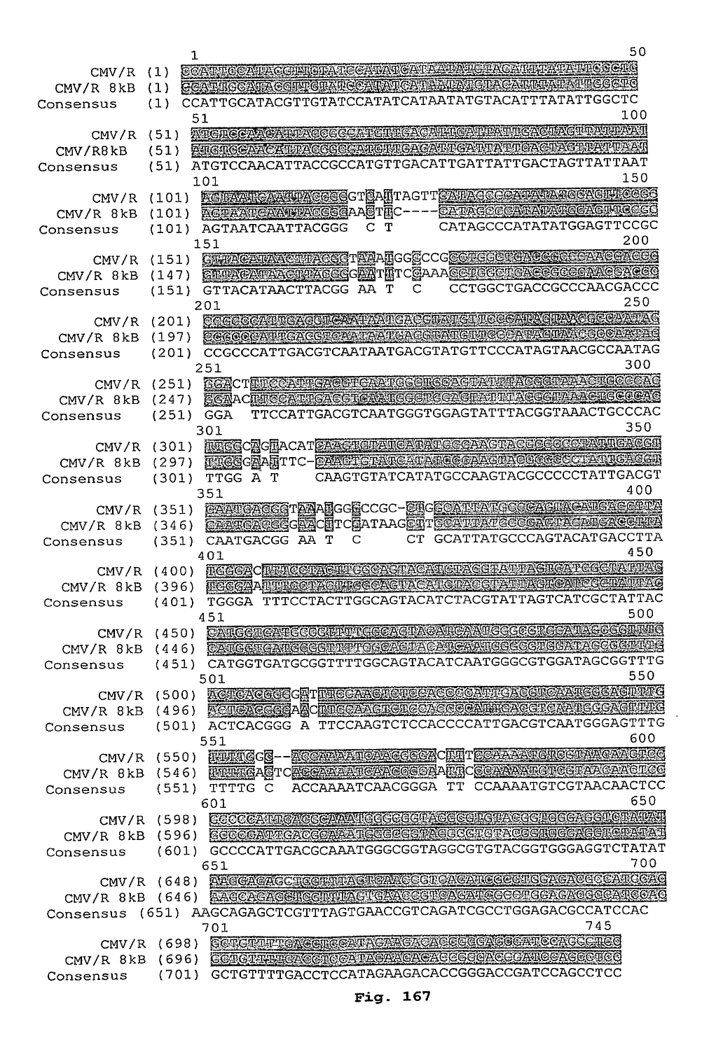

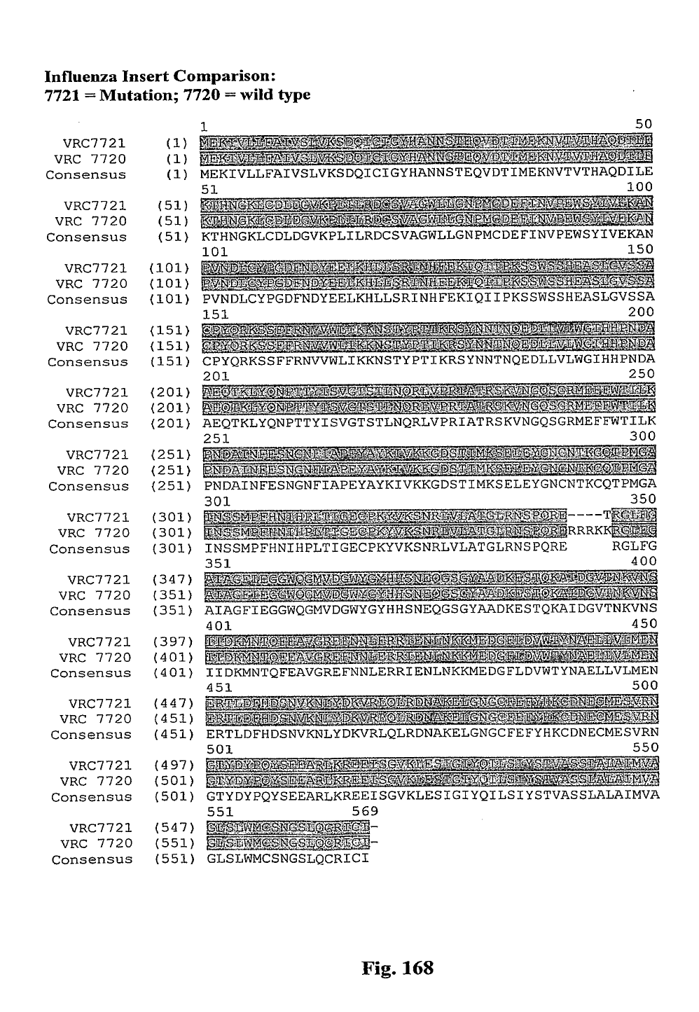

The HA gene of human influenza viruses contains multiple basic amino acid residues at the HA1/HA2 cleavage site similar to that seen in highly pathogenic avian influenza viruses. One component of our vaccine design was to delete this stretch of basic amino acids and to convert the HA to a low-pathogenic form without alteration of its antigenicity. The HA genes of wild type isolates were modified at the cDNA level so that the first five basic amino acid residues present in the cleavage site of wild type virus HA were deleted. In addition, a threonine residue was added proximal to the cleavage site to resemble that found in low-pathogenic avian strains (e.g., FIG. 168). This mutation is denoted HA (dPC-a), HA mut A or mutant A.

In some embodiments denoted “short” HA genes, we truncated the carboxy end (trans-membrane) part of the HA protein. The short HA version is truncated 10 amino acids upstream from the trans-membrane region.

In other embodiments, denoted “long” HA genes, we also truncated the carboxy end (trans-membrane) part of the HA protein. Long HA genes have ten (10) more amino acids than the corresponding short versions. The long version is truncated right before the trans-membrane region of the HA. Long HA constructs contain ten more amino acids upstream of the trans-membrane region of HA than that of the short HA version.

Some embodiments of the invention also have a “spacer”. The same spacer sequence is always used: When extra functional regions (e.g., Foldon domain, His Tag, etc.) are added to any naturally existing protein (e.g., HA), extra amino acids may be added between the regions to provide extra physical space, commonly called a spacer. The spacer is mainly for different functional regions to properly fold to their functional structural motifs without hindering each others' region.

Some embodiments denoted TT-M2(dTM) gene encode an influenza matrix 2 gene that has a transmembrane deletion.

Other embodiments denoted /h contain an influenza gene that is codon optimized for humans.

Some embodiments are denoted “Foldon-His”. In order to obtain the HA protein in its more native form, a foldon region is added to help the HA protein monomers to form the native trimer molecule. The His region acts as a tag for identification purposes of the HA protein and facilitates isolation of the HA protein by using anti-His antibodies, such as by the use of anti-His column chromatography.

### Part 2

### Definitions

Unless defined otherwise, technical and scientific terms used herein have the same meaning as commonly understood by one of ordinary skill in the art to which this invention belongs. See, e.g., Singleton P and Sainsbury D., in Dictionary of Microbiology and Molecular Biology 3rd ed., J. Wiley & Sons, Chichester, N.Y., 2001; and Fields Virology 5th ed., Knipe D. M. and Howley P. M. eds, Lippincott Williams & Wilkins, a Wolters Kluwer Business, Philadelphia 2007.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

### Nucleic Acid Molecules

As indicated herein, nucleic acid molecules of the present invention may be in the form of RNA or in the form of DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Nucleic acid molecules of the present invention include DNA molecules comprising an open reading frame (ORF), also termed “insert”, of a wild-type influenza gene; and DNA molecules which comprise a sequence substantially different from those described above but which, due to the degeneracy of the genetic code, still encode an ORF of a wild-type influenza polypeptide. Of course, the genetic code is well known in the art. Degenerate variants optimized for human codon usage are preferred

In another aspect, the invention provides a nucleic acid molecule comprising a polynucleotide which hybridizes under stringent hybridization conditions to a portion of the polynucleotide in a nucleic acid molecule of the invention described above. By “stringent hybridization conditions” is intended overnight incubation at 42° C. in a solution comprising: 50% formamide, 5 times SSC (750 mM NaCl, 75 mM trisodium citrate), 50 mM sodium phosphate (pH 7.6), 5 times Denhardt's solution, 10% dextran sulfate, and 20 μg/ml denatured, sheared salmon sperm DNA, followed by washing the filters in 0.1 times SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide.

By a portion of a polynucleotide of “at least 20 nt in length,” for example, is intended 20 or more contiguous nucleotides from the nucleotide sequence of the reference polynucleotide. Of course, a polynucleotide which hybridizes only to a complementary stretch of T (or U) resides, would not be included in a polynucleotide of the invention used to hybridize to a portion of a nucleic acid of the invention, since such a polynucleotide would hybridize to any nucleic acid molecule containing a poly T (or U) stretch or the complement thereof (e.g., practically any double-stranded DNA clone).

As indicated herein, nucleic acid molecules of the present invention which encode an influenza polypeptide may include, but are not limited to those encoding the amino acid sequence of the full-length polypeptide, by itself, the coding sequence for the full-length polypeptide and additional sequences, such as those encoding a leader or secretory sequence, such as a pre-, or pro- or prepro-protein sequence, the coding sequence of the full-length polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example, ribosome binding and stability of mRNA; and additional coding sequence which codes for additional amino acids, such as those which provide additional functionalities.

The present invention further relates to variants of the nucleic acid molecules of the present invention, which encode portions, analogs or derivatives of the influenza protein. Variants may occur naturally, such as a natural allelic variant. By an “allelic variant” is intended one of several alternate forms of a gene occupying a given locus on a genome of an organism (Genes II, Lewin, B., ed., John Wiley & Sons, New York (1985)). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both: Alterations in the coding regions may produce conservative or non-conservative amino acid substitutions, deletions or additions. Especially preferred among these are silent substitutions, additions and deletions, which do not alter the properties and activities of the influenza polypeptide or portions thereof. Also especially preferred in this regard are conservative substitutions.

Further embodiments of the invention include nucleic acid molecules comprising a polynucleotide having a nucleotide sequence at least 95% identical, and more preferably at least 96%, 97%, 98% or 99% identical to a nucleotide sequence encoding a polypeptide having the amino acid sequence of a wild-type influenza polypeptide or a nucleotide sequence complementary thereto.

By a polynucleotide having a nucleotide sequence at least, for example, 95% “identical” to a reference nucleotide sequence encoding an influenza polypeptide is intended that the nucleotide sequence of the polynucleotide is identical to the reference sequence except that the polynucleotide sequence may include up to five point mutations per each 100 nucleotides of the reference nucleotide sequence encoding the influenza polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 95%, 96%, 97%, 98% or 99% identical to the reference nucleotide sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, 1981 Advances in Applied Mathematics 2:482-489, to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 95%, 96%, 97%, 98% or 99% identical to the nucleic acid sequences shown herein in the Sequence Listing which encode a polypeptide having influenza polypeptide activity. By “a polypeptide having influenza activity” is intended polypeptides exhibiting influenza activity in a particular biological assay. For example, HA, NA, NP and M2 protein activity can be measured for changes in immunological character by an appropriate immunological assay.

Of course, due to the degeneracy of the genetic code, one of ordinary skill in the art will immediately recognize that a large number of the nucleic acid molecules having a sequence at least 95%, 96%, 97%, 98%, or 99% identical to a nucleic acid sequence shown herein in the Sequence Listing will encode a polypeptide “having influenza polypeptide activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having influenza polypeptide activity. This is because the skilled artisan is fully aware of amino acid substitutions that are either less likely or not likely to significantly affect protein function (e.g., replacing one aliphatic amino acid with a second aliphatic amino acid).

For example, guidance concerning how to make phenotypically silent amino acid substitutions is provided in Bowie, J. U. et al. 1990 Science 247:1306-1310, wherein the authors indicate that proteins are surprisingly tolerant of amino acid substitutions.

### Polypeptides and Fragments

The invention further provides an influenza polypeptide having the amino acid sequence encoded by an open reading frame (ORF), also termed “insert”, of a wild-type influenza gene, or a peptide or polypeptide comprising a portion thereof (e.g., HA, NA, NP and M2).

It will be recognized in the art that some amino acid sequences of the influenza polypeptides can be varied without significant effect of the structure or function of the protein. If such differences in sequence are contemplated, it should be remembered that there will be critical areas on the protein which determine activity.

Thus, the invention further includes variations of the influenza polypeptides which show substantial influenza polypeptide activity or which include regions of influenza proteins such as the protein portions discussed below. Such mutants include deletions, insertions, inversions, repeats, and type substitutions. As indicated, guidance concerning which amino acid changes are likely to be phenotypically silent can be found in Bowie, J. U., et al. 1990 Science 247:1306-1310.

Thus, the fragment, derivative or analog of the polypeptide of the invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

As indicated, changes are preferably of a minor nature, such as conservative amino acid substitutions that do not significantly affect the folding or activity of the protein (see Table 3).

TABLE 3
Conservative Amino Acid Substitutions

| | |
|---|---|
| Aromatic | Phenylalanine |
| | Tryptophan |
| | Tyrosine |
| Ionizable: Acidic | Aspartic Acid |
| | Glutamic Acid |
| Ionizable: Basic | Arginine |
| | Histidine |
| | Lysine |
| Nonionizable Polar | Asparagine |
| | Glutamine |
| | Selenocystine |
| | Serine |
| | Threonine |
| Nonpolar (Hydrophobic) | Alanine |
| | Glycine |
| | Isoleucine |
| | Leucine |
| | Proline |
| | Valine |
| Sulfur Containing | Cysteine |
| | Methionine |

Of course, the number of amino acid substitutions a skilled artisan would make depends on many factors, including those described above. Generally speaking, the number of amino acid substitutions for any given influenza polypeptide will not be more than 50, 40, 30, 20, 10, 5 or 3.

Amino acids in the influenza polypeptides of the present invention that are essential for function can be identified by methods known in the art, such as site-directed mutagenesis or alanine-scanning mutagenesis (Cunningham and Wells, 1989 Science 244:1081-1085). The latter procedure introduces single alanine mutations at every residue in the molecule. The resulting mutant molecules are then tested for biological activity such as changes in immunological character.

The polypeptides of the present invention are conveniently provided in an isolated form. By “isolated polypeptide” is intended a polypeptide removed from its native environment. Thus, a polypeptide produced and/or contained within a recombinant host cell is considered isolated for purposes of the present invention.

Also intended as an “isolated polypeptide” are polypeptides that have been purified, partially or substantially, from a recombinant host cell or a native source. For example, a recombinantly produced version of the influenza polypeptide can be substantially purified by the one-step method described in Smith and Johnson, 1988 Gene 67:31-40.

The polypeptides of the present invention include a polypeptide comprising a polypeptide encoded by a nucleic acid sequence shown herein in the Sequence Listing; as well as polypeptides which are at least 95% identical, and more preferably at least 96%, 97%, 98% or 99% identical to those described above and also include portions of such polypeptides with at least 30 amino acids and more preferably at least 50 amino acids.

By a polypeptide having an amino acid sequence at least, for example, 95% “identical” to a reference amino acid sequence of an influenza polypeptide is intended that the amino acid sequence of the polypeptide is identical to the reference sequence except that the polypeptide sequence may include up to five amino acid alterations per each 100 amino acids of the reference amino acid of the influenza polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 95%, 96%, 97%, 98% or 99% identical to, for instance, the amino acid sequence encoded by a nucleic acid sequence shown herein in the Sequence Listing can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the fill length of the reference amino acid sequence and that gaps in homology of up to 5% of the total number of amino acid residues in the reference sequence are allowed.

The polypeptides of the invention may be produced by any conventional means. Houghten, R. A. 1985 Proc Natl Acad Sci USA 82:5131-5135. This “Simultaneous Multiple Peptide Synthesis (SMPS)” process is further described in U.S. Pat. No. 4,631,211 to Houghten et al (1986).

The present invention also relates to vectors which include the nucleic acid molecules of the present invention, host cells which are genetically engineered with the recombinant vectors, and the production of influenza polypeptides or fragments thereof by recombinant techniques.

The polynucleotides may be joined to a vector, which serves as a “backbone”, containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs and cytomegalovirus (CMV) such as the CMV immediate early promoter, to name a few. Other suitable promoters will be known to the skilled artisan. The expression constructs will further contain sites for transcription initiation, termination and, in the transcribed region, a ribosome binding site for translation The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The influenza polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

### Pharmaceutical Formulations, Dosages, and Modes of Administration

The compounds of the invention may be administered using techniques well known to those in the art. Preferably, compounds are formulated and administered by genetic immunization. Techniques for formulation and administration may be found in “Remington's Pharmaceutical Sciences”, 18^{th} ed., 1990, Mack Publishing Co., Easton, Pa. Suitable routes may include parenteral delivery, such as intramuscular, intradermal, subcutaneous, intramedullary injections, as well as, intrathecal, direct intraventricular, intravenous, intraperitoneal, intranasal, or intraocular injections, just to name a few. For injection, the compounds of the invention may be formulated in aqueous solutions, preferably in physiologically compatible buffers such as Hanks' solution, Ringer's solution, or physiological saline buffer.

In instances wherein intracellular administration of the compounds of the invention is preferred, techniques well known to those of ordinary skill in the art may be utilized. For example, such compounds may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are effectively delivered into the cell cytoplasm.

Nucleotide sequences of the invention which are to be intracellularly administered may be expressed in cells of interest, using techniques well known to those of skill in the art. For example, expression vectors derived from viruses such as retroviruses, adenoviruses, adeno-associated viruses, herpes viruses, vaccinia viruses, polio viruses, or sindbis or other RNA viruses, or from plasmids may be used for delivery and expression of such nucleotide sequences into the targeted cell population. Methods for the construction of such expression vectors are well known. See, for example, Molecular Cloning: a Laboratory Manual, 3^{rd} edition, Sambrook et al. 2001 Cold Spring Harbor Laboratory Press, and Current Protocols in Molecular Biology, Ausubel et al. eds., John Wiley & Sons, 1994.

The invention extends to the use of a plasmid for primary immunization (priming) of a host and the subsequent use of a recombinant virus, such as a retrovirus, adenovirus, adeno-associated virus, herpes virus, vaccinia virus, polio virus, or sindbis or other RNA virus, for boosting said host, and vice versa. For example, the host may be immunized (primed) with a plasmid by DNA immunization and receive a boost with the corresponding viral construct, and vice versa. Alternatively, the host may be immunized (primed) with a plasmid by DNA immunization and receive a boost with not the corresponding viral construct but a different viral construct, and vice versa.

With respect to influenza virus HA, NA, NP and M2, protein sequences of the invention, they may be used as therapeutics or prophylatics (as subunit vaccines) in the treatment of influenza virus infection. A therapeutically effective dose refers to that amount of the compound sufficient to result in amelioration of symptoms or a prolongation of survival in a patient. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50ED50. Compounds which exhibit large therapeutic indices are preferred. The data obtained from cell culture assays and animal studies can be used in formulating a range of dosage for use in humans. The dosage of such compounds lies preferably within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (e.g., the concentration of the test compound which achieves a half-maximal inhibition of viral infection relative to the amount of the event in the absence of the test compound) as determined in cell culture. Such information can be used to more accurately determine useful doses in humans. Levels in plasma may be measured, for example, by high performance liquid chromatography (HPLC).

The compounds of the invention may, further, serve the role of a prophylactic vaccine, wherein the host produces antibodies and/or CTL responses against influenza virus HA, NA, NP or M2 protein, which responses then preferably serve to neutralize influenza viruses by, for example, inhibiting further influenza infection. Administration of the compounds of the invention as a prophylactic vaccine, therefore, would comprise administering to a host a concentration of compounds effective in raising an immune response which is sufficient to elicit antibody and/or CTL responses to influenza virus HA, NA, NP or M2 protein, and/or neutralize an influenza virus, by, for example, inhibiting the ability of the virus to infect cells. The exact concentration will depend upon the specific compound to be administered, but may be determined by using standard techniques for assaying the development of an immune response which are well known to those of ordinary skill in the art.

The compounds may be formulated with a suitable adjuvant in order to enhance the immunological response. Such adjuvants may include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum.

Adjuvants suitable for co-administration in accordance with the present invention should be ones that are potentially safe, well tolerated and effective in people including QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (see Kim et al., 2000, Vaccine, 18: 597 and references therein).

Other contemplated adjuvants that may be administered include lectins, growth factors, cytokines and lymphokines such as alpha-interferon, gamma-interferon, platelet derived growth factor (PDGF), gCSF, GMCSF, TNF, epidermal growth factor (EGF), IL-1, IL-2, IL-4, IL-6, IL-8, IL-10 and IL-12.

For all such treatments described above, the exact formulation, route of administration and dosage can be chosen by the individual physician in view of the patient's condition. (See e.g., Fingl et al., 1975, in “The Pharmacological Basis of Therapeutics”, Ch. 1 p. 1).

It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the viral infection of interest will vary with the severity of the condition to be treated and the route of administration. The dose and perhaps prime-boost regimen, will also vary according to the age, weight, and response of the individual patient. A program comparable to that discussed above may be used in veterinary medicine.

The pharmacologically active compounds of this invention can be processed in accordance with conventional methods of galenic pharmacy to produce medicinal agents for administration to patients, e.g., mammals including humans.

The compounds of this invention can be employed in admixture with conventional excipients, i.e., pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, which includes intramuscular, intradermal, subcutaneous, intranasal, intracapsular, intraspinal, intrasternal, and intravenous injection, particularly suitable are injectable, sterile solutions, preferably oily or aqueous solutions, as well as suspensions, emulsions, or implants, including suppositories. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For enteral application, particularly suitable are tablets, dragees, liquids, drops, suppositories, or capsules. The pharmaceutical compositions may be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze dry the new compounds and use the lyophilizates obtained, for example, for the preparation of products for injection.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Genetic Immunization

Genetic immunization according to the present invention elicits an effective immune response without the use of infective agents or infective vectors. Vaccination techniques which usually do produce a CTL response do so through the use of an infective agent. A complete, broad based immune response is not generally exhibited in individuals immunized with killed, inactivated or subunit vaccines. The present invention achieves the full complement of immune responses in a safe manner without the risks and problems associated with vaccinations that use infectious agents.

According to the present invention, DNA or RNA that encodes a target protein is introduced into the cells of an individual where it is expressed, thus producing the target protein. The DNA or RNA is linked to regulatory elements necessary for expression in the cells of the individual. Regulatory elements for DNA include a promoter and a polyadenylation signal. In addition, other elements, such as a Kozak region, may also be included in the genetic construct.

The genetic constructs of genetic vaccines comprise a nucleotide sequence that encodes a target protein operably linked to regulatory elements needed for gene expression. Accordingly, incorporation of the DNA or RNA molecule into a living cell results in the expression of the DNA or RNA encoding the target protein and thus, production of the target protein.

When taken up by a cell, the genetic construct which includes the nucleotide sequence encoding the target protein operably linked to the regulatory elements may remain present in the cell as a functioning extrachromosomal molecule or it may integrate into the cell's chromosomal DNA. DNA may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Since integration into the chromosomal DNA necessarily requires manipulation of the chromosome, it is preferred to maintain the DNA construct as a replicating or non-replicating extrachromosomal molecule. This reduces the risk of damaging the cell by splicing into the chromosome without affecting the effectiveness of the vaccine. Alternatively, RNA may be administered to the cell. It is also contemplated to provide the genetic construct as a linear minichromosome including a centromere, telomeres and an origin of replication.

The necessary elements of a genetic construct of a genetic vaccine include a nucleotide sequence that encodes a target protein and the regulatory elements necessary for expression of that sequence in the cells of the vaccinated individual. The regulatory elements are operably linked to the DNA sequence that encodes the target protein to enable expression.

The molecule that encodes a target protein is a protein-encoding molecule which is translated into protein. Such molecules include DNA or RNA which comprise a nucleotide sequence that encodes the target protein. These molecules may be cDNA, genomic DNA, synthesized DNA or a hybrid thereof or an RNA molecule such as mRNA. Accordingly, as used herein, the terms “DNA construct”, “genetic construct” and “nucleotide sequence” are meant to refer to both DNA and RNA molecules.

The regulatory elements necessary for gene expression of a DNA molecule include: a promoter, an initiation codon, a stop codon, and a polyadenylation signal. In addition, enhancers are often required for gene expression. It is necessary that these elements be operable in the vaccinated individual. Moreover, it is necessary that these elements be operably linked to the nucleotide sequence that encodes the target protein such that the nucleotide sequence can be expressed in the cells of a vaccinated individual and thus the target protein can be produced.

Initiation codons and stop codons are generally considered to be part of a nucleotide sequence that encodes the target protein. However, it is necessary that these elements are functional in the vaccinated individual.

Similarly, promoters and polyadenylation signals used must be functional within the cells of the vaccinated individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego, Calif.), referred to as the SV40 polyadenylation signal, can be used. Additionally, the bovine growth hormone (bgh) polyadenylation signal can serve this purpose.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with a mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration.

An additional element may be added which serves as a target for cell destruction if it is desirable to eliminate cells receiving the genetic construct for any reason. A herpes thymidine kinase (tk) gene in an expressible form can be included in the genetic construct. When the construct is introduced into the cell, tk will be produced. The drug gangcyclovir can be administered to the individual and that drug will cause the selective killing of any cell producing tk. Thus, a system can be provided which allows for the selective destruction of vaccinated cells.

In order to be a functional genetic construct, the regulatory elements must be operably linked to the nucleotide sequence that encodes the target protein. Accordingly, it is necessary for the initiation and termination codons to be in frame with the coding sequence.

Open reading frames (ORFs) encoding the protein of interest and another or other proteins of interest may be introduced into the cell on the same vector or on different vectors. ORFs on a vector may be controlled by separate promoters or by a single promoter. In the latter arrangement, which gives rise to a polycistronic message, the ORFs will be separated by translational stop and start signals. The presence of an internal ribosome entry site (IRES) site between these ORFs permits the production of the expression product originating from the second ORF of interest, or third, etc. by internal initiation of the translation of the bicistronic or polycistronic mRNA.

According to the invention, the genetic vaccine may be administered directly into the individual to be immunized or ex vivo into removed cells of the individual which are reimplanted after administration. By either route, the genetic material is introduced into cells which are present in the body of the individual. Routes of administration include, but are not limited to, intramuscular, intraperitoneal, intradermal, subcutaneous, intravenous, intraarterially, intraoccularly and oral as well as transdermally or by inhalation or suppository. Preferred routes of administration include intramuscular, intraperitoneal, intradermal and subcutaneous injection. Genetic constructs may be administered by means including, but not limited to, traditional syringes, needleless injection devices, or microprojectile bombardment gene guns. Alternatively, the genetic vaccine may be introduced by various means into cells that are removed from the individual. Such means include, for example, ex vivo transfection, electroporation, microinjection and microprojectile bombardment. After the genetic construct is taken up by the cells, they are reimplanted into the individual. It is contemplated that otherwise non-immunogenic cells that have genetic constructs incorporated therein can be implanted into the individual even if the vaccinated cells were originally taken from another individual.

The genetic vaccines according to the present invention comprise about 1 nanogram to about 1000 micrograms of DNA. In some preferred embodiments, the vaccines contain about 10 nanograms to about 800 micrograms of DNA. In some preferred embodiments, the vaccines contain about 0.1 to about 500 micrograms of DNA. In some preferred embodiments, the vaccines contain about 1 to about 350 micrograms of DNA. In some preferred embodiments, the vaccines contain about 25 to about 250 micrograms of DNA. In some preferred embodiments, the vaccines contain about 100 micrograms DNA.

The genetic vaccines according to the present invention are formulated according to the mode of administration to be used. One having ordinary skill in the art can readily formulate a genetic vaccine that comprises a genetic construct. In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vaso-constriction agent is added to the formulation. The pharmaceutical preparations according to the present invention are provided sterile and pyrogen free.

Genetic constructs may optionally be formulated with one or more response enhancing agents such as: compounds which enhance transfection, i.e., transfecting agents; compounds which stimulate cell division, i.e., replication agents; compounds which stimulate immune cell migration to the site of administration, i.e., inflammatory agents; compounds which enhance an immune response, i.e., adjuvants or compounds having two or more of these activities.

In one embodiment, bupivacaine, a well known and commercially available pharmaceutical compound, is administered prior to, simultaneously with or subsequent to the genetic construct. Bupivacaine and the genetic construct may be formulated in the same composition. Bupivacaine is particularly useful as a cell stimulating agent in view of its many properties and activities when administered to tissue. Bupivacaine promotes and facilitates the uptake of genetic material by the cell. As such, it is a transfecting agent. Administration of genetic constructs in conjunction with bupivacaine facilitates entry of the genetic constructs into cells. Bupivacaine is believed to disrupt or otherwise render the cell membrane more permeable. Cell division and replication is stimulated by bupivacaine. Accordingly, bupivacaine acts as a replicating agent. Administration of bupivacaine also irritates and damages the tissue. As such, it acts as an inflammatory agent which elicits migration and chemotaxis of immune cells to the site of administration. In addition to the cells normally present at the site of administration, the cells of the immune system which migrate to the site in response to the inflammatory agent can come into contact with the administered genetic material and the bupivacaine. Bupivacaine, acting as a transfection agent, is available to promote uptake of genetic material by such cells of the immune system as well.

In addition to bupivacaine, mepivacaine, lidocaine, procains, carbocaine, methyl bupivacaine, and other similarly acting compounds may be used as response enhancing agents. Such agents act as cell stimulating agents which promote the uptake of genetic constructs into the cell and stimulate cell replication as well as initiate an inflammatory response at the site of administration.

Other contemplated response enhancing agents which may function as transfecting agents and/or replicating agents and/or inflammatory agents and which may be administered include lectins, growth factors, cytokines and lymphokines such as alpha-interferon, gamma-interferon, platelet derived growth factor (PDGF), gCSF, gMCSF, TNF, epidermal growth factor (EGF), IL-1, IL-2, IL-4, IL-6, IL-8, IL-10 and IL-12 as well as collagenase, fibroblast growth factor, estrogen, dexamethasone, saponins, surface active agents such as immune-stimulating complexes (ISCOMS), Freund's incomplete adjuvant, LPS analog including monophosphoryl Lipid A (MPL), muramyl peptides, quinone analogs and vesicles such as squalene and squalane, hyaluronic acid and hyaluronidase may also be administered in conjunction with the genetic construct. In some embodiments, combinations of these agents are co-administered in conjunction with the genetic construct. In other embodiments, genes encoding these agents are included in the same or different genetic construct(s) for co-expression of the agents.

With respect to influenza virus HA, NA, NP and M2 nucleotide sequences of the invention, particularly through genetic immunization, may be used as therapeutics or prophylatics in the treatment of influenza virus infection. A therapeutically effective dose refers to that amount of the compound sufficient to result in amelioration of symptoms or a prolongation of survival in a patient. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from cell culture assays and animal studies can be used in formulating a range of dosage for use in humans. The dosage of such compounds lies preferably within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the (e.g., the concentration of the test compound which achieves a half-maximal inhibition of viral infection relative to the amount of the event in the absence of the test compound) as determined in cell culture. Such information can be used to more accurately determine useful doses in humans. Levels in plasma may be measured, for example, by high performance liquid chromatography (HPLC).

The compounds (for genetic immunization) of the invention may, further, serve the role of a prophylactic vaccine, wherein the host produces antibodies and/or CTL responses against influenza virus HA, NA, NP and M2, which responses then preferably serve to neutralize influenza viruses by, for example, inhibiting further influenza infection. Administration of the compounds of the invention as a prophylactic vaccine, therefore, would comprise administering to a host a concentration of compounds effective in raising an immune response which is sufficient to elicit antibody and/or CTL responses to influenza virus HA, NA, NP and M2 and/or neutralize influenza virus, by, for example, inhibiting the ability of the virus to infect cells. The exact concentration will depend upon the specific compound to be administered, but may be determined by using standard techniques for assaying the development of an immune response which are well known to those of ordinary skill in the art.

### Prime and Boost Immunization Regimes

The present invention relates to “prime and boost” immunization regimes in which the immune response induced by administration of a priming composition is boosted by administration of a boosting composition. For example, effective boosting can be achieved using replication-defective adenovirus vectors, following priming with any of a variety of different types of priming compositions. The present invention employs replication-deficient adenovirus which have been found to be an effective means for providing a boost to an immune response primed to antigen using any of a variety of different priming compositions.

Replication-deficient adenovirus derived from human serotype 5 has been developed as a live viral vector by Graham and colleagues (Graham & Prevec 1995 Mol Biotechnol 3:207-20; and Bett et al. 1994 PNAS USA 91:8802-6). Adenoviruses are non-enveloped viruses containing a linear double-stranded DNA genome of around 3600 bp. Recombinant viruses can be constructed by in vitro recombination between an adenovirus genome plasmid and a shuttle vector containing the gene of interest together with a strong eukaryotic promoter, in a permissive cell line which allows viral replication. High viral titers can be obtained from the permissive cell line, but the resulting viruses, although capable of infecting a wide range of cell types, do not replicate in any cells other than the permissive line, and are therefore a safe antigen delivery system. Recombinant adenoviruses have been shown to elicit protective immune responses against a number of antigens including tick-borne encephalitis virus NSI protein (Jacobs et al. 1992 J Virol 66:2086-95) and measles virus nucleoprotein (Fooks et al. 1995 Virology 210:456-65).

Use of embodiments of the present invention allows for recombinant replication-defective adenovirus expressing an antigen to boost an immune response primed by a DNA vaccine. Replication-defective adenoviruses induce an immune response after intramuscular immunization. In prime/boost vaccination regimes the replication-defective adenovirus is also envisioned as being able to prime a response that can be boosted by a different recombinant virus or recombinantly produced antigen.

Non-human primates immunized with plasmid DNA and boosted with replication-defective adenovirus are protected against challenge. Both recombinant replication-deficient adenovirus and plasmid DNA are vaccines that are safe for use in humans. Advantageously, a vaccination regime using intramuscular immunization for both prime and boost can be employed, constituting a general immunization regime suitable for inducing an immune response, e.g., in humans.

The present invention in various aspects and embodiments employs a replication-deficient adenovirus vector encoding an antigen for boosting an immune response to the antigen primed by previous administration of the antigen or nucleic acid encoding the antigen.

A general aspect of the present invention provides for the use of a replication-deficient adenoviral vector for boosting an immune response to an antigen.

One aspect of the present invention provides a method of boosting an immune response to an antigen in an individual, the method including provision in the individual of a replication-deficient adenoviral vector including nucleic acid encoding the antigen operably linked to regulatory sequences for production of antigen in the individual by expression from the nucleic acid, whereby an immune response to the antigen previously primed in the individual is boosted.

An immune response to an antigen may be primed by genetic immunization, by infection with an infectious agent, or by recombinantly produced antigen.

A further aspect of the invention provides a method of inducing an immune response to an antigen in an individual, the method comprising administering to the individual a priming composition comprising the antigen or nucleic acid encoding the antigen and then administering a boosting composition which comprises a replication-deficient adenoviral vector including nucleic acid encoding the antigen operably linked to regulatory sequences for production of antigen in the individual by expression from the nucleic acid.

A further aspect provides for use of a replication-deficient adenoviral vector, as disclosed, in the manufacture of a medicament for administration to a mammal to boost an immune response to an antigen. Such a medicament is generally for administration following prior administration of a priming composition comprising the antigen or nucleic acid encoding the antigen.

The priming composition may comprise any viral vector, including adenoviral, or other than adenoviral, such as a vaccinia virus vector such as a replication-deficient strain such as modified virus Ankara (MVA) (Mayr et al. 1978 Zentralbi Bakteriol 167:375-90; Sutter and Moss 1992 PNAS USA 89:10847-51; Sutter et al. 1994 Vaccine 12:1032-40) or NYVAC (Tartaglia et al. 1992 Virology 118:217-32), an avipox vector such as fowlpox or canarypox, e.g., the strain known as ALVAC (Kanapox, Paoletti et al. 1994 Dev Biol Stand 1994 82:65-9), or a herpes virus vector.

The priming composition may comprise DNA encoding the antigen, such DNA preferably being in the form of a circular plasmid that is not capable of replicating in mammalian cells. Any selectable marker should not be resistant to an antibiotic used clinically, so for example Kanamycin resistance is preferred to Ampicillin resistance. Antigen expression should be driven by a promoter which is active in mammalian cells, for instance the cytomegalovirus immediate early (CMV IE) promoter.

In particular embodiments of the various aspects of the present invention, administration of a priming composition is followed by boosting with first and second boosting compositions, the first and second boosting compositions being the same or different from one another, e.g., as exemplified below. Still further boosting compositions may be employed without departing from the present invention. In one embodiment, a triple immunization regime employs DNA, then adenovirus (Ad) as a first boosting composition, and then MVA as a second boosting composition, optionally followed by a further (third) boosting composition or subsequent boosting administration of one or other or both of the same or different vectors. Another option is DNA then MVA then Ad, optionally followed by subsequent boosting administration of one or other or both of the same or different vectors.

The antigen to be included in respective priming and boosting compositions (however many boosting compositions are employed) need not be identical, but should share epitopes. The antigen may correspond to a complete antigen in a target pathogen or cell, or a fragment thereof. Peptide epitopes or artificial strings of epitopes may be employed, more efficiently cutting out unnecessary protein sequence in the antigen and encoding sequence in the vector or vectors. One or more additional epitopes may be included, for instance epitopes which are recognized by T helper cells, especially epitopes recognized in individuals of different HLA types.

Within the replication-deficient adenoviral vector, regulatory sequences for expression of the encoded antigen will include a promoter. By “promoter” is meant a sequence of nucleotides from which transcription may be initiated of DNA operably linked downstream (i.e., in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter. Other regulatory sequences including terminator fragments, polyadenylation sequences, enhancer sequences, marker genes, internal ribosome entry site (IRES) and other sequences may be included as appropriate, in accordance with the knowledge and practice of the ordinary person skilled in the art: see, for example, Molecular Cloning: a Laboratory Manual, 3^{rd} edition, Sambrook et al. 2001 Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1994.

Suitable promoters for use in aspects and embodiments of the present invention include the cytomegalovirus immediate early (CMV IE) promoter, with or without intron A, and any other promoter that is active in mammalian cells.

Either or both of the priming and boosting compositions may include an adjuvant or cytokine, such as alpha-interferon, gamma-interferon, platelet-derived growth factor (PDGF), granulocyte macrophage-colony stimulating factor (gM-CSF) granulocyte-colony stimulating factor (gCSF), tumor necrosis factor (TNF), epidermal growth factor (EGF), IL-1, IL-2, IL-4, IL-6, IL-8, IL-10 and IL-12, or encoding nucleic acid therefor Administration of the boosting composition is generally weeks or months after administration of the priming composition, preferably about 2-3 weeks or 4 weeks, or 8 weeks, or 16 weeks, or 20 weeks, or 24 weeks, or 28 weeks, or 32 weeks.

Preferably, administration of priming composition, boosting composition, or both priming and boosting compositions, is intramuscular immunization.

Intramuscular administration of adenovirus vaccines or plasmid DNA may be achieved by using a needle to inject a suspension of the virus or plasmid DNA. An alternative is the use of a needless injection device to administer a virus or plasmid DNA suspension (using, e.g., Biojector™) or a freeze-dried powder containing the vaccine (e.g., in accordance with techniques and products of Powderject), providing for manufacturing individually prepared doses that do not need cold storage. This would be a great advantage for a vaccine that is needed in third world countries or undeveloped regions of the world.

Adenovirus is a virus with an excellent safety record in human immunizations. The generation of recombinant viruses can be accomplished simply, and they can be manufactured reproducibly in large quantities. Intramuscular administration of recombinant replication-deficient adenovirus is therefore highly suitable for prophylactic or therapeutic vaccination of humans against diseases which can be controlled by an immune response.

The individual may have a disease or disorder such that delivery of the antigen and generation of an immune response to the antigen is of benefit or has a therapeutically beneficial effect.

Most likely, administration will have prophylactic aim to generate an immune response against a pathogen or disease before infection or development of symptoms.

Diseases and disorders that may be treated or prevented in accordance with the present invention include those in which an immune response may play a protective or therapeutic role.

Components to be administered in accordance with the present invention may be formulated in pharmaceutical compositions. These compositions may comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g., intravenous, cutaneous or subcutaneous, intramucosal (e.g., gut), intranasal, intramuscular, or intraperitoneal routes.

As noted, administration is preferably intradermal, subcutaneous or intramuscular.

Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at an intramuscular site, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

A slow-release formulation may be employed.

Following production of replication-deficient adenoviral particles and optional formulation of such particles into compositions, the particles may be administered to an individual, particularly human or other primate.

Administration may be to another animal, e.g., an avian species or a mammal such as a mouse, rat or hamster, guinea pig, rabbit, sheep, goat, pig, horse, cow, donkey, dog or cat.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated Prescription of treatment, e.g., decisions on dosage etc., is within the responsibility of general practitioners and other medical doctors, or in a veterinary context a veterinarian, and typically takes account of the disorder to be treated, the condition of the individual patient, the site of delivery, the method of administration and other factors known to practitioners. Examples of the techniques and protocols mentioned above can be found in Remington's Pharmaceutical Sciences”, 18^{th} ed., 1990, Mack Publishing Co., Easton, Pa.

In one preferred regimen, DNA is administered (preferably intramuscularly) at a dose of 10 micrograms to 50 milligrams/injection, followed by adenovirus (preferably intramuscularly) at a dose of 5×10⁷−1×10¹² particles/injection.

The composition may, if desired, be presented in a kit, pack or dispenser, which may contain one or more unit dosage forms containing the active ingredient. The kit, for example, may comprise metal or plastic foil, such as a blister pack. The kit, pack, or dispenser may be accompanied by instructions for administration.

A composition may be administered alone or in combination with other treatments, either simultaneously or sequentially dependent upon the condition to be treated.

Delivery to a non-human mammal need not be for a therapeutic purpose, but may be for use in an experimental context, for instance in investigation of mechanisms of immune responses to an antigen of interest, e.g., protection against disease.

### Part 3

### Protective Immunity to Lethal Challenge of the 1918 Pandemic Influenza Virus by Vaccination

The remarkable infectivity and virulence of the 1918 influenza virus resulted in an unprecedented pandemic, raising the question of whether it is possible to develop protective immunity to this virus and whether immune evasion may have contributed to its spread. Here, we report that the highly lethal 1918 virus is susceptible to immune protection by a preventive vaccine, and we define its mechanism of action. Immunization with plasmid expression vectors encoding hemagglutinin (HA) elicited potent CD4 and CD8 cellular responses as well as neutralizing antibodies. Antibody specificity and titer were defined by a microneutralization and a pseudotype assay that could assess antibody specificity without the need for high-level biocontainment. This pseudotype inhibition assay can define evolving serotypes of influenza viruses and facilitate the development of immune sera and neutralizing monoclonal antibodies that may help contain pandemic influenza. Notably, mice vaccinated with 1918 HA plasmid DNAs showed complete protection to lethal challenge. T cell depletion had no effect on immunity, but passive transfer of purified IgG from anti-H1(1918) immunized mice provided protective immunity for naive mice challenged with infectious 1918 virus. Thus, humoral immunity directed at the viral HA can protect against the 1918 pandemic virus.

### Introduction

In the past century, three influenza outbreaks have caused significant increases in human fatalities throughout the world, the hallmark of pandemics. Among them, the 1918 strain was notable for its exceptional infectivity and disease severity in otherwise healthy individuals, with mortality of 40 million to 50 million people worldwide. Through molecular analysis of preserved specimens, it has been possible to characterize the gene products of this virus in an effort to determine the molecular basis for its immunopathogenesis (Stevens J et al. 2006 J Mol Biol 355:1143-1155; Taubenberger J K et al. 2005 Nature 437:889-893; Glaser L et al. 2005 J Virol 79:11533-11536; Reid A H 2004 J Virol 78:12462-12470; Kobasa D 2004 Nature 431:703-707; Tumpey T M et al. 2004 Proc Natl Acad Sci USA 101:3166-3171; Stevens J et al. 2004 Science 303:1866-1870; Gamblin S J et al. 2004 Science 303:1838-1842; Basler C F et al. 2001 Proc Natl Acad Sci USA 98:2746-2751; Reid A H et al. 2000 Proc Natl Acad Sci USA 97:6785-6790). Recently, this virus was reconstructed fully from lung specimens stored in 1918 (Stevens J et al. 2006 J Mol Biol 355:1143-1155; Taubenberger J K et al. 2005 Nature 437:889-893; Glaser L et al. 2005 J Virol 79:11533-11536; Reid A H 2004 J Virol 78:12462-12470; Kobasa D 2004 Nature 431:703-707; Tumpey T M et al. 2004 Proc Natl Acad Sci USA 101:3166-3171; Stevens J et al. 2004 Science 303:1866-1870; Gamblin S J et al. 2004 Science 303:1838-1842; Basler C F et al. 2001 Proc Natl Acad Sci USA 98:2746-2751; Reid A H et al. 2000 Proc Natl Acad Sci USA 97:6785-6790) and shown to cause highly lethal infection in mice. This model has provided an opportunity to analyze the genetic basis of its virulence and to explore mechanisms of immunity relevant to the development of vaccines and antivirals for this and other influenza viruses with pandemic potential. Specifically, we sought to determine whether it is possible to generate protective immunity to this virus, to define potential mechanisms of immune protection, and to ascertain whether countermeasures can be developed to contain outbreaks.

### Results

### Generation of Expression Vectors

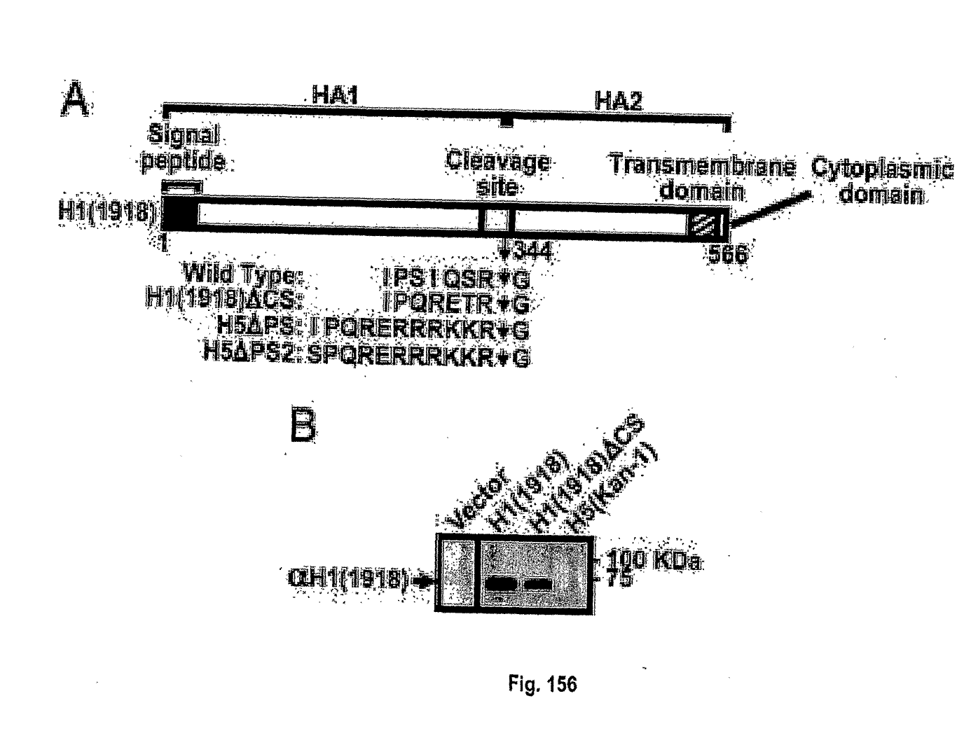

To evaluate the protective immune response against the 1918 influenza virus, synthetic plasmid expression vectors encoding HA were generated. Plasmids expressing wild-type (WT) hemagglutinin (HA) (GenBank accession no. DQ868374) or HA with a mutation in the HA cleavage site (GenBank no. DQ868375) that attenuates influenza virus during viral replication were prepared by using nonviral codons, both to ensure compatibility with mammalian codon usage and to exclude the unlikely possibility of homologous reassortment with WT influenza viruses that might generate replication-competent H1(1918) virus (FIG. 156A). Expression was confirmed in transfected human renal 293T cells by Western blot analysis using antisera from mice vaccinated with these DNA expression vectors (FIG. 156B).

Referring to FIG. 155, expression of viral HAs is depicted. In FIG. 156A, the structure of the vectors, together with the indicated specific mutations in the cleavage site, for immunogens and lentiviral vector pseudotypes is shown. In FIG. 156B, expression of the indicated viral HAs was determined by Western blot analysis with antisera reactive to the 1918 influenza HA. Expression was evaluated after transfection of the indicated plasmids in 293T cells. Arrows indicate the relevant viral HA bands.

Vaccination with DNA Vaccines and Analysis of Cellular Immune Responses

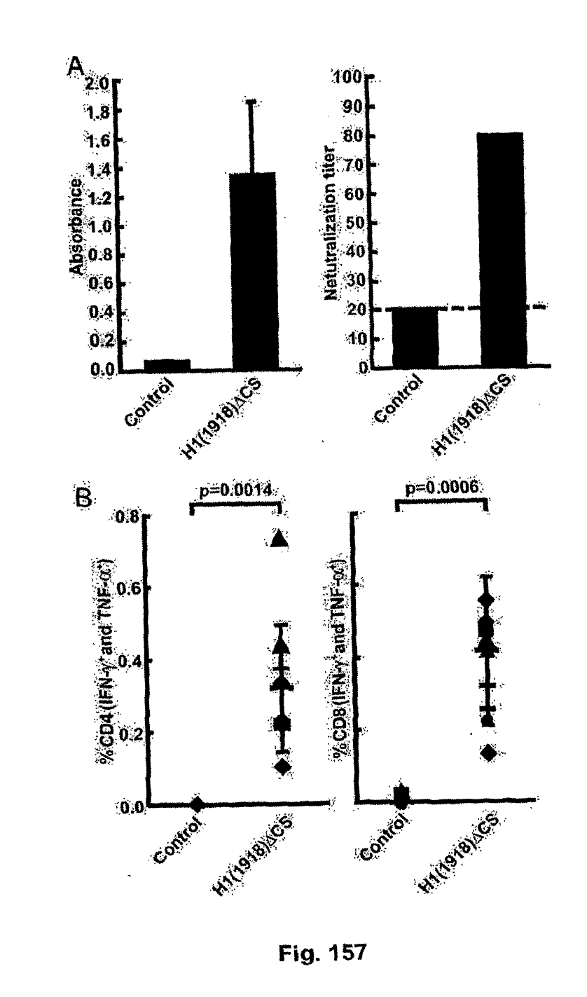

Antisera to the 1918 HA were generated by intramuscular inoculation of BALB/c mice with DNA vaccines. DNA vaccines included the WT 1918 HA as well as an attenuated HA (1918) cleavage site (ACS) mutant (FIG. 156A). Immunization with HA induced significant cellular and humoral responses. For example, H1(1918)ACS induced a >10-fold increase in H1(1918)-specific antibody measured by ELISA (FIG. 157A Left). The neutralization activity was confirmed by the microneutralization assay using live 1918 (H1N1) virus (FIG. 157A Right). Neutralization titers of ≈1:80 were observed with both the WT and mutant HA expression vectors using this assay, with a modest increase in the neutralization titers observed with the attenuated cleavage site mutant. Next, the cellular immune response was characterized in immunized mice. Vaccinated animals showed a marked increase in H1(1918) antigen-specific CD4 and CD8 T cells (FIG. 157B), as determined by intracellular cytokine staining to measure cells synthesizing either IFN-γ and/or TNF-α, at levels at least 62-fold and 122-fold above the background response for each T cell subset, respectively. These responses compared favorably with those observed with other viral spike proteins, including HIV, severe acute respiratory syndrome (SARS), coronavirus, and Ebola virus, all of which contain a number of predicted T cell epitopes.

Referring to FIG. 157, humoral and cellular immune responses to 1918 influenza HA after DNA vaccination are indicated. In FIG. 157A, antibody responses induced by DNA vaccination against the 1918 influenza HA measured by ELISA (Left) or microneutralization (Right) are shown. The microneutralization assay using dilutions of heat-inactivated sera and titers of virus neutralizing antibody were determined as the reciprocal of the highest dilution of serum that neutralized 100 plaque-forming units of virus in Madin-Darby canine kidney (MDCK) cell cultures on a 96-well plate (Right). ELISA for viral nucleocapsid protein (NP) was performed for determining the presence of the virus as the read-out. Data are presented as the mean for each group. In FIG. 157B, intracellular cytokine staining was performed to analyze the T cell response to viral HA peptides. The percentage of activated T cells that produced either IFN-γ and/or TNF-α in response to stimulation with overlapping peptides in CD4 (Left) or CD8 (Right) is shown. Lymphocytes from mice (n=5 per group) immunized with empty plasmid vector (control) or mice (n=10 per group) immunized with the indicated plasmid at 0, 3, 6, and 12 weeks were assessed, and immune responses were measured 11 days after the final boost. Nonstimulated cells gave responses similar to controls at background levels. Symbols indicate the response of individual animals, and the median value is shown with a horizontal bar.

### Immune Protection Conferred by DNA Vaccination and Mechanism of Action

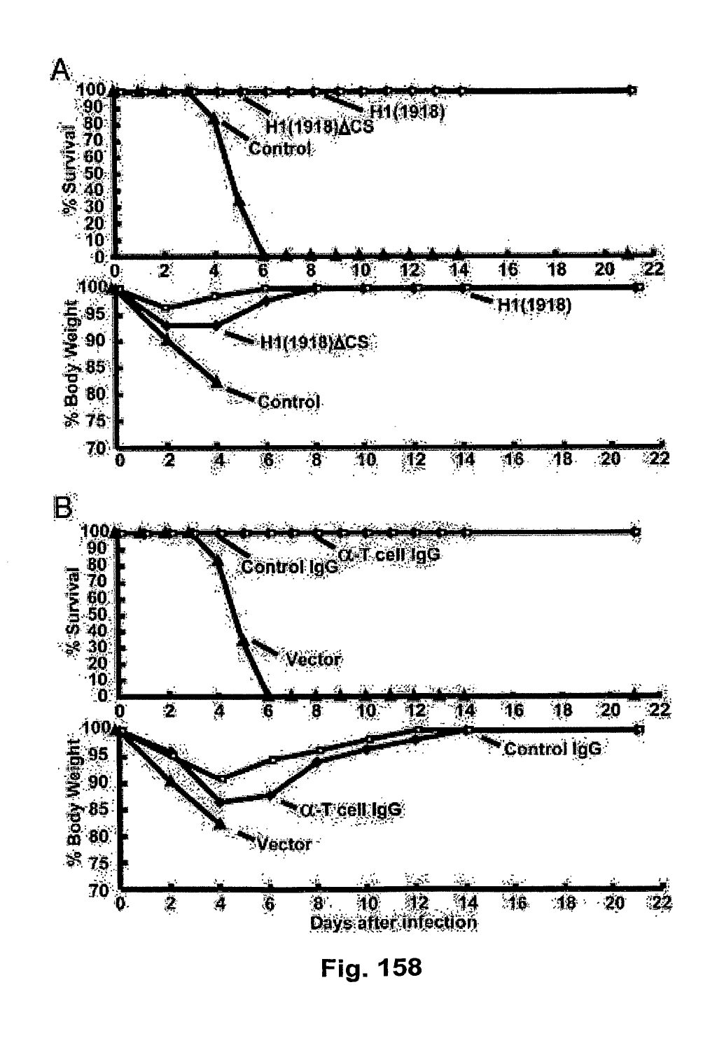

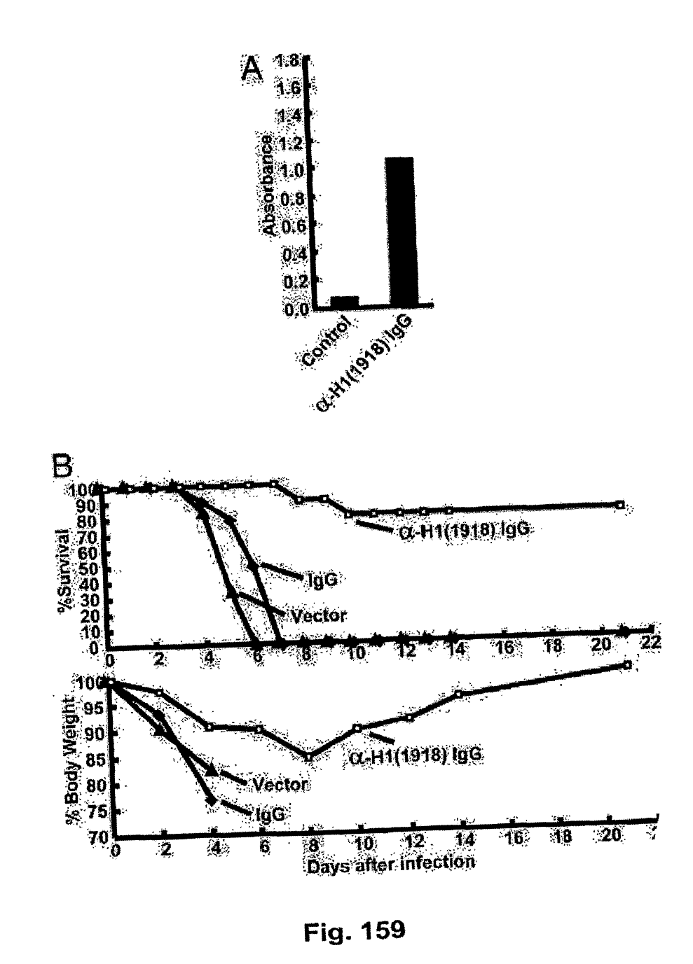

To assess the efficacy of this vaccine against lethal infection by the 1918 influenza virus, vaccinated animals were given 100 LD50 of live virus intranasally 14 days after the final DNA plasmid injection. All studies with live reconstructed 1918 virus were performed under high-containment (biosafety level 3 enhanced (BSL3)) laboratory conditions in accordance with guidelines of the National Institutes of Health and the Centers for Disease Control (Tumpey TM et al. 2005 Science 310:77-80 and the world-wide-web at cdc.gov/flu/h2n2bsl3.htm). Both the WT and cleavage mutant H1(1918) plasmids induced complete protection against lethal viral challenge measured by survival (FIG. 158A Upper) as well as extent of weight loss compared with controls (FIG. 158A Lower). To define the mechanism of immune protection, T cell depletion was performed with monoclonal antibodies (anti-mouse CD4(GK1.5), anti-mouse CD8(2.43), or anti-mouse CD90(30-H12)) to CD4, CD8, and CD90 (Thy1.2), previously shown to deplete >99% T cells in lung and spleen (Yang Z-Y et al. 2004 Nature 428:561-564). The same negative control DNA plasmid vectors without an insert were used for both the DNA vaccine and the depletion studies. T cell depletion of H1(1918) immunized animals did not affect survival (FIG. 158B Upper), and these mice showed weight loss comparable with nonimmune Ig-treated animals (FIG. 158B Lower). In contrast, when IgG from immunized mice purified by Protein A chromatography was passively transferred to naive recipients, neutralizing antibodies could be detected in the recipients at levels slightly below those of the immunized mice (FIG. 159A vs. FIG. 157A Left). Importantly, passive transfer of this immune IgG conferred immune protection in 8 of 10 mice, compared with 0 of 10 animals that received IgG from control unvaccinated animals (FIG. 159B; P=0.0007).

Referring to FIG. 158, immune protection conferred against lethal challenge of 1918 influenza and lack of T cell dependence is shown. In FIG. 158A, immunization with H1(1918), H1(1918)ΔCS, or negative control plasmid expression vectors was performed in mice (n=10 per group) as described (Tumpey T M et al. 2005 Science 310:77-80), and survival (Upper) and weight loss (Lower) were evaluated. The statistical significance between these groups are P=1.08×10⁻⁵ and P=1.08×10⁻⁵ with respect to controls by Fisher's exact test. In FIG. 158B, monoclonal rat anti-mouse anti-CD4, CD8, and CD90 (T cell depletion) were used to deplete T cells in H1(1918)ACS immunized mice, in comparison with a control group of vaccinated animals injected with nonimmune IgG (Control IgG). Vector-immunized animals that received no depletion served as additional controls (Vector). Mice were administered IgG at −3, +3, +9, and +15 days after viral challenge. Mice (n=10 per group) were then evaluated for survival (Upper) and weight loss (Lower). No decrease in immune protection was observed in T cell-depleted animals.

Referring to FIG. 159, immune mechanism of protection showing dependence on Ig is shown. In FIG. 159A, the activity of control, nonimmune, IgG (control), or anti-HA immune IgG (anti-H1(1918)), purified as described (Yang Z-Y et al. 2004 Nature 428:561-564), was confirmed by ELISA before passive transfer into naive recipients (n=10 per group). Passive transfer is depicted in FIG. 159B. To assess the protective effects of immune IgG, mice received immune or control IgG 24 h before infection with 100 LD50 of 1918 virus. Mice were then monitored for survival and weight loss throughout a 21-day observation period. The difference between the immune (α-H1(1918) IgG) and control IgG (IgG) groups was significant (P=0.0007).

### Development of Pseudotyped Lentiviral Reporters

The functional activity of this HA was assessed through the use of a pseudotyped lentiviral vector in which the 1918 HA was used instead of the retroviral envelope. The HA pseudoviruses were then characterized for their susceptibility to neutralizing antibodies by using a luciferase reporter gene. Whereas H5-pseudotyped. lentiviral vectors readily mediated entry, the H1(1918) strain was inactive (FIG. 160A Left vs. Center, second column). Because H5 viruses contain a cleavage site recognized more broadly by proteases, the protease cleavage site region from H5 was substituted into the relevant sequence of H1(1918) in an effort to increase its processing to a fusion-competent form. A modification that extended 11 aa (FIG. 156A; H5ΔPS2) from the cleavage site improved entry more than a slightly shorter 9-aa (FIG. 156A; H5ΔPS) addition (FIG. 160A Center, third and fourth column). Insertion of an H5 cleavage site conferred a similar increase in entry for an independent H1 strain, PR/8 (FIG. 160A Right), suggesting that such modifications could allow otherwise incompetent HAs to generate functionally active pseudotyped vectors that could be used to assess neutralizing antibody activity.

Humoral immunity in mice immunized with 1918 HA plasmid DNAs was assessed by using the viral pseudotype assay. To analyze the ability of antibodies to neutralize virus, the pseudoviruses were incubated with antisera from control and HA-immune animals, and the reduction in luciferase activity was measured. Sera from animals immunized with the H1(1918) or H5(Kan-1) HA expression vectors neutralized pseudotyped lentiviral vectors encoding the homologous, but not the heterologous, HAs at dilutions of 1:400 in this assay, in contrast to nonimmune sera, which had no effect (FIG. 160B). These titers were considerably higher than those measured by microneutralization (FIG. 157A) or hemagglutination inhibition, suggesting that the pseudotype vector inhibition assay is more sensitive. Thus, immunity in the lethal challenge model was readily measured and correlated with protection in this assay.

Referring to FIG. 160, HA-pseudotyped lentiviral vectors were developed. In FIG. 160A, gene transfer mediated by lentiviral vectors pseudotyped with H1(1918), H5(Kan-1), or other HAs containing the H5 protease cleavage site was measured with a luciferase reporter assay. In FIG. 160B, neutralization by antisera from mice immunized with the indicated HA plasmid expression vectors or no insert (control) plasmid DNA vectors was measured with the luciferase assay with the HA-pseudotyped lentiviral vectors. Reduction of gene transfer in the presence of immune sera was observed in a dose-dependent fashion.

### Discussion

In this study, gene-based vaccination was used to elicit cellular and humoral immune responses to the 1918 influenza virus HA. The humoral immune response was able to neutralize this virus, and these antibodies were necessary and sufficient to confer protective immunity to lethal challenge by virus. In contrast, although a robust T cell response was observed, this response was not required for protective immunity. Whereas slightly increased weight loss was observed in T cell-depleted animals relative to controls, this difference was also observed to some extent in control animals, likely reflecting stress associated with the additional manipulations. Thus, although it remains possible that T cells may contribute to an antiviral effect and could potentially contribute to cross-heterotypic protection to variant viruses, they are not required for immune protection for this vaccine in the mouse. In humans, immune control is likely also antibody dependent, but we cannot exclude the possibility that cellular mechanisms of protection may contribute to viral clearance. The unique circumstances that contributed to the 1918 pandemic spread are also unknown. Although there has been speculation about the types of viruses that may have circulated before the epidemic and their implications for herd immunity, neither the virus isolates nor sera from these times are available, and the present epidemiologic data do not permit further analysis, although recovery of such viruses through methods used for the 1918 rescue could be informative in the future.

The ability to use a pseudotyped lentiviral vector with viral HA allows for analysis of neutralizing antibody response with increased sensitivity in the detection of neutralizing antibodies compared with the traditional antiviral assay. In addition, the ability to perform screening in the absence of replication-competent virus allows for methods to screen for neutralizing antibodies and to generate antiviral reagents, for the 1918 pandemic influenza virus, as well as avian H5N1 influenza virus and other possibly highly pathogenic influenza viruses in a conventional biosafety level 2 laboratory. It will also be desirable in the future to compare results from this assay with hemagglutination inhibition to explore its ability to predict protective immunity in humans.

Further testing is envisioned as confirming that DNA vaccination can confer similar humoral immune responses in humans. Previous experience has shown that this mode of vaccination is somewhat less robust in humans than in rodents. The results nonetheless demonstrate that humoral immune responses are protective against viral infection and provide proof of concept that immunization with H1(1918), whether by gene-based vaccines, recombinant proteins, or inactivated virus, is likely to be successful for the generation of protective immunity in humans. These data also suggest that there is no intrinsic immune resistance of this pandemic influenza virus. This knowledge, together with the enhanced ability to measure and screen for neutralizing antibody as a correlate of protection, will facilitate the development of novel protective vaccines and monoclonal antibodies for the 1918 influenza virus and for contemporary pandemic flu threats.

### Materials and Methods

### Immunogen and Plasmid Construction

Plasmids encoding different versions of HA protein [A/South Carolina/1/18, GenBank AF117241; A/Thailand/1(KAN-1)/2004, GenBank AAS65615; A/PR/8/34, GenBank P03452] were synthesized by using human-preferred codons as described (Yang Z-Y et al. 2004 Nature 428:561-564) by GeneArt (Regensburg, Germany). All of the H1 and H5 HA cleavage-site mutants were made with the original viral cleavage amino acid sequences changed to PQRETRG (SEQ ID NO: 156), ΔCS, which is originally from a low-pathogen city H5 isolate (A/chicken/Mexico/31381/94; GenBank AAL34297), and the modification resulted in the trypsin-dependent phenotype (Li S et al. 1999 J Infect Dis 179:1132-1138) and may alter the antigenic character. To make the pseudotyped lentiviral vector for H1(1918), the original viral cleavage site was changed to IPQRERRRKKRG (SEQ ID NO: 157), ΔPS, and SPQRERRRKKRG (SEQ ID NO: 158), ΔPS2, which is originally from a highly virulent H5 (A/Thailand/1(KAN-1)/2004), and the modification should result in the trypsin-independent phenotype. Protein expression was confirmed by Western blot analysis (Kong WP et al. 2003 J Virol 77:12764-12772) with serum from mice immunized with HA-expressing plasmid DNA.

To synthesize the coating antigen for the ELISA, codon-optimized cDNA corresponding to aa 1 to 530 was cloned into CMV/R 8κB expression vector for efficient expression in mammalian cells. This vector utilizes the CMVIR plasmid backbone (Barouch D H et al. 2005 J Virol 79:8828-8834) with several modifications at the NF-κB binding sites in the enhancer/promoter region to enhance immunogenicity of the inserts expressed in the plasmid DNA constructs. Four κB binding sites in the enhancer/promoter region were modified by two pairs to a consensus κB sequence (Leung T H et al. 2004 Cell 118:453464) as follows: nucleotide (nt) 802 GCACCAAAATCAACGGGACTTT (SEQ ID NO: 159) was changed to ACTCACCAAAATCAACGGGAATTC (SEQ ID NO: 160); nt 753 GGGGATTT (SEQ ID NO: 167) was changed to GGGACTT (SEQ ID NO: 168); nt 648 GGGACTTT (SEQ ID NO: 169) was changed to GGGAATTT (SEQ ID NO: 170); and nt 607 TAAATGGCCCGCCTG (SEQ ID NO: 171) was changed to GAACTTCCATAAGCTT (SEQ ID NO: 172). Two additional such sites were introduced upstream of the original enhancer/promoter region as follows: nt 550 GGCAGTACATCA (SEQ ID NO: 173) was changed to GGGAATTTCCA (SEQ ID NO: 174); nt 497 GGGACTTTC (SEQ ID NO: 175) was changed to GGGAACTTC (SEQ ID NO: 176); nt 714 TAAATGGCGGG (SEQ ID NO: 177) was changed to GAATTTCCAAA (SEQ ID NO: 178); and nt 361 GGGGTCATTAGTT (SEQ ID NO: 179) was changed to GGGAACTTC (SEQ ID NO: 180). When tested in mice, a CMV/R 8κB plasmid induced a higher immunological response to HIV clade B envelope immunogen, with higher antigen-specific CD4 and CD8 T cell responses than CMV/R, and also improved antibody responses. A trimerization sequence from bacteriophage T4 fibritin was introduced followed by a thrombin cleavage site and a His tag at the C terminus. The plasmid was then transfected into 293T cells, and the cell media containing the secreted protein was collected and purified by nickel column chromatography. The purified protein contains the following additional residues at the C terminus (RSLVPRGSPGSGYIPEAPRDGQAYVRKDGEWVLLSTFLGHHHHHH) (SEQ ID NO: 181), where the thrombin site is in italics, the fibritin trimerization sequence is underlined, and the His tag is in bold. Similar modifications used for HA molecule structural studies have been described (Stevens J et al. 2004 Science 303:1866-1870).

### Vaccination

Female BALB/c mice (6-8 weeks old; Jackson Laboratories, Bar Harbor, ME) were immunized intramuscularly with 15 μg of plasmid DNA in 100 μl of PBS (pH 7.4) at weeks 0, 3, and 6 for T lymphocyte depletion, IgG passive transfer, and viral challenge. T cell depletion and antibody transfer were performed as described below. An additional immunization at week 12 was performed in groups for the intracellular cytokine staining assay.

### Flow Cytometric Analysis of Intracellular Cytokines

CD4+ and CD8+ T cell responses were evaluated by using intracellular cytokine staining for IFN-γ and TNF-α as described (Kong WP et al. 2003 J Virol 77:12764-12772) with peptide pools (15 mers overlapping by 11 aa, 2.5 μg/ml each) covering the HA protein. Cells were then fixed, permeabilized, and stained by using rat monoclonal anti-mouse CD3, CD4, CD8, IFN-γ, and TNF-α (BD-PharMingen, San Diego, Calif.). The IFN-γ- and TNF-α-positive cells in the CD4+ and CD8+ cell populations were analyzed with the program FlowJo (Tree Star, Ashland, Oreg.).

### ELISA for Mouse Anti-HA IgG and IgM

The mouse anti-HA IgG and IgM ELISA titer was measured by using a described method (Yang Z-Y et al. 2004 J Virol 78:4029-4036). Purified HA protein was made by purification of a transmembrane-domain-truncated HA protein with a trimerization domain, thrombin cleavage site, and His tag expressed in the CMV/R 8κB expression vector. H1 or H5 protein was purified from transfected 293T cell culture supernatants as detailed in Immunogen and Plasmid Construction, also analogous to a described method (Stevens J et al. 2004 Science 303:1866-1870), and used to coat the plate.

### Production of HA Pseudotyped Lentiviral Vectors and Measurement of Neutralizing Activity of Immune Serum

Influenza HA-pseudotyped lentiviral vectors expressing a luciferase reporter gene were produced as described (Yang Z-Y et al. 2004 J Virol 78:4029-4036). Briefly, 293T cells were cotransfected by using the following plasmids: 7 μg of pCMVΔR8.2, 7 μg of pHR′CMV-Luc, and 125 ng CMV/R 8κB H1(1918), H1(1918)(ΔPS), H1(1918)(ΔPS2), or H1(PR/8)(ΔPS), or H5(Kan-1). Cells were transfected overnight, washed, and replenished with fresh medium. Forty-eight hours later, supernatants were harvested, filtered through a 0.45-μm syringe filter, aliquotted, and used immediately or frozen at −80° C. For neutralization assays, antisera were mixed with 100 μl of pseudoviruses at various dilutions and added to 293A cells (Invitrogen, Carlsbad, Calif.) in 48-well dishes (30,000 cells per well). Plates were washed, and fresh medium was added 14-16 h later. Forty-eight hours after infection, cells were lysed in mammalian cell lysis buffer (Promega, Madison, Wis.). A standard quantity of cell lysate was used in a luciferase assay with luciferase assay reagent (Promega) according to the manufacturer's protocol.

### Microneutralization Assay of 1918 (H1N1) by Mouse Immune Antisera

Two-fold dilutions of heat-inactivated sera were tested in a microneutralization assay for the presence of antibodies that neutralized the infectivity of 100 TCID50 (50% tissue culture infectious dose) of 1918 (HINI) viruses on MDCK cell monolayers by using two wells per dilution on a 96-well plate as described (Rowe T et al. 1999 J Clin Microbiol 37:937-943). After 2 days of incubation, cells were fixed, and ELISA was performed to detect the presence of viral nucleoprotein (NP) and determine the neutralization activity.

Challenge of Mice with Live 1918 (H1N1) Virus

Two weeks after final vaccination, mice were challenged intranasally with 100 LD50 of 1918 (HINI) virus in a volume of 50 μl. After infection, mice were monitored daily for disease signs and death for 21 days after infection Individual body weights and death were recorded for each group on various days after inoculation. All 1918 influenza virus studies were performed under high-containment biosafety level 3 enhanced (BSL3) as described (Tumpey T M et al. 2005 Science 310:77-80).

### Depletion of T Cell Subsets in Vivo

To deplete specific T cell subsets, known rat monoclonal antibodies (anti-mouse CD4 (GK1.5), anti-mouse CD8(2.43), or anti-mouse CD90(30-H12)), prepared as described (Yang Z-Y et al. 2004 Nature 428:561-564; Epstein S L et al. 2005 Vaccine 23:5404-5410) and obtained from the National Cell Culture Center (Minneapolis, Minn.), were administered i.p. (1 mg each in 1 ml of PBS) 3 days before challenge and 3, 9, and 15 days after the viral challenge. For nonimmune Ig treatment (control), an isotype-matched anti-human leukocyte antigen (SFR3-D5) mononclonal antibody was used.

### Passive Transfer of Ig

IgG from mice immunized with plasmid DNA encoding HAACS (immune) or no insert (controls) was purified from sera by using a Montage Antibody Purification Kit (Millipore, Billerica, Mass.), and antibody titer was confirmed by ELISA. Briefly, 0.3 ml of purified IgG (from≈0.7 ml of serum) was administered i.v. into each recipient naïve mouse (n=10 per group) by tail vein injection 24 h before challenge.

### Statistical Analysis

Each individual animal immune response was counted as an individual value for statistical analysis. The significance of the cellular and humoral immune responses was calculated by Student's t test (tails=2, type=2) as indicated by the P value. For immune protection between groups, Fisher's exact test was used to analyze the data, and the result was indicated by the P value.

### Part 4

Previous Experience with CMV/R Promoter VRC-1012 Plasmid Backbone DNA Vaccines

VRC vaccines utilizing a VRC-1012 plasmid backbone with the translational enhancer element of the Human T-cell Leukemia Virus Long Terminal Repeat (the R element) substituted for a portion of the Cytomegalovirus (CMV) 5′ untranslated region have undergone standard preclinical safety testing (biodistribution and repeated-dose toxicity). Two vaccines constructed in this backbone have been evaluated in nonclinical GLP toxicology and biodistribution studies, followed by Phase I clinical studies under BB-IND 11995 (SARS) (n=10 subjects) and BB-IND 11294 (Ebola) (n=21 subjects). In addition, the CMV/R promoter was allowed into initial clinical testing of an HIV vaccine (BB-IND 11750) based on prior human experience with the Ebola vaccine (BB-IND 11294), without new preclinical safety studies required. This HIV vaccine product has advanced to Phase 11 testing (BB-IND 12326) as part of a DNA prime-recombinant adenovector boost regimen. It has been administered to 55 subjects at the VRC Clinic and is currently enrolling in three international studies. The preclinical and clinical experience with VRC vaccines in the CMV/R promoter/VRC-1012 plasmid backbone suggests that modifications to the inserted gene do not significantly impact vaccine biodistribution. Furthermore human clinical safety data with this promoter have now been obtained in over 100 human subjects under several INDs, as summarized below.

Descriptions of Modified Influenza Plasinid DNA Vaccines The new influenza vaccine products utilize the 1012 plasmid backbone constructed with a CMV /R 8κB promoter that has not yet been tested in humans, but is very similar to the CMV/R promoter that has been tested in over 100 human subjects (see below). The sequences of both the CMV/R and CMV/R 8κB promoters are compared below.

The family of transcription factors, NF-κB, plays an essential role in inflammatory and immunological responses. Members of the NF-κB family finction by binding to their DNA binding site in the promoter/enhancer region of the genes that they regulate. Several NF-κB binding sites in the CMV/R 8κB promoter have been modified to incorporate optimal κB sites in an effort to enhance immunogenicity of the constructs There are four NF-κB binding sites in the CMV promoter/enhancer. In an effort to further improve the CMV/R DNA expression system, it was rationalized that optimization of these binding sites to a consensus sequence by minor nucleotide changes might enhance their ability to induce immunological responses.

The CMV/R 8κB plasmid was evaluated in mice for its ability to induce immunological responses to the HIV envelope gp 145 (ΔCFI)(ΔV12)(Bal) immunogen. Five mice were vaccinated with 2.5 μg plasmid DNA at Weeks 0, 3, and 6. Ten days after the last vaccination, serum and spleens were collected for antigen specific ELISA and T-cell response analyses. The results (see below) showed that the new CMV/R 8κB vector could generate statistically higher antigen specific CD4 and CD8 T-cell responses than CMV/R, and also improved antibody responses. Similar changes in the mouse model have shown improved immunogenicity when tested in non-human primates (Barouch, D. H. et al. 2005 J Virol 79:8828-8834).

### VRC Influenza Plasniid DNA Vaccines

Three new vaccines have been developed, each composed of a single plasmid DNA encoding hemaggluinin (HA) protein from H1N1, H3N2 and H5N1 subtypes isolated from recent human outbreaks of influenza. The HI protein (A/New Caledonia/20/99/H1N1) expressed by the VRC vaccine has been administered to humans as a component Of the currently licensed Influenza Virus Vaccine Fluzone®. The H3 protein (A/Wyoming/3/03/H3N2) was recommended for use by the CDC for the 2004-2005 flu season (CDC 2005 MMWR Morb Mortal Wkly Rep 54(RR-8): 1-40). The H5 ((A/Thailand/1 (KAN-1)/2004 (H5N1) has been administered to humans in clinical trials of inactivated H5N1 influenza vaccine (NIAID press release). The sources of the HA gene sequences used in the production of the plasmid DNA vaccines are summarized in Table 4 below.

Plasmid VRC-7727 encodes Influenza HA H1, VRC-7729 encodes HA H3, and VRC-7721 encodes HA H5. For each construct, the plasmid encodes a modified HA protein with a mutation at the protease cleavage site. The original viral cleavage sequence was changed from wild-type of the original strain to PQRETRG (SEQ ID NO: 182) which is originally from a non-pathogenic H5 isolate (A/chicken/Mexico/31381/94) and other non-pathogenic strains with the same amino acid sequence. This mutated sequence makes the HA protein less accessible to cleavage by cellular proteases (e.g., trypsin, furin) which is one of the most critical steps for viral infection.

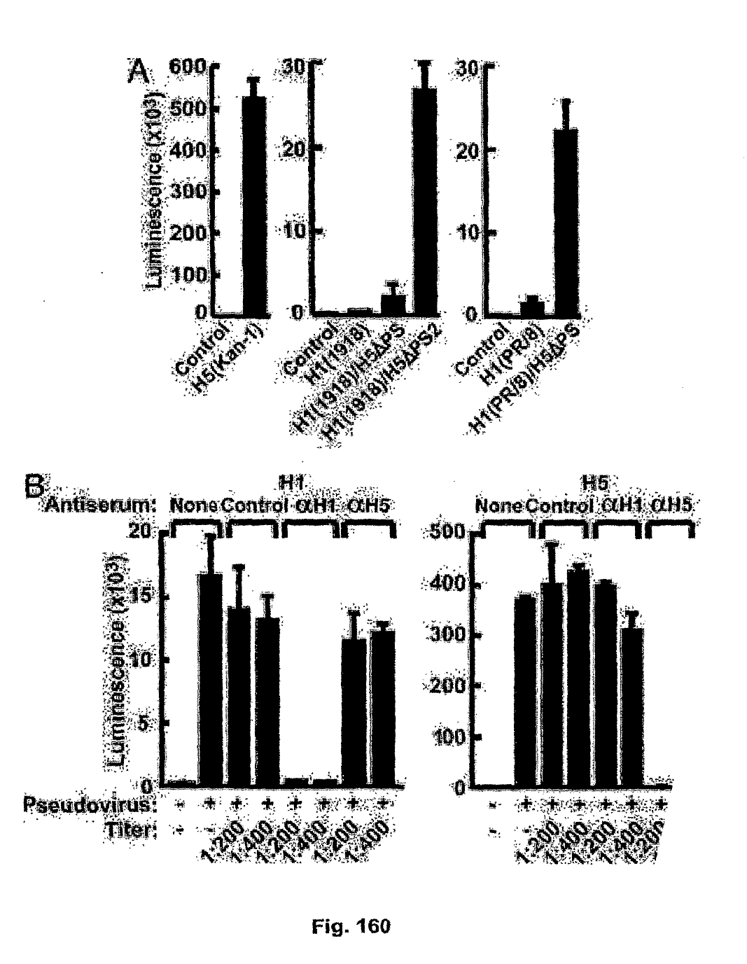

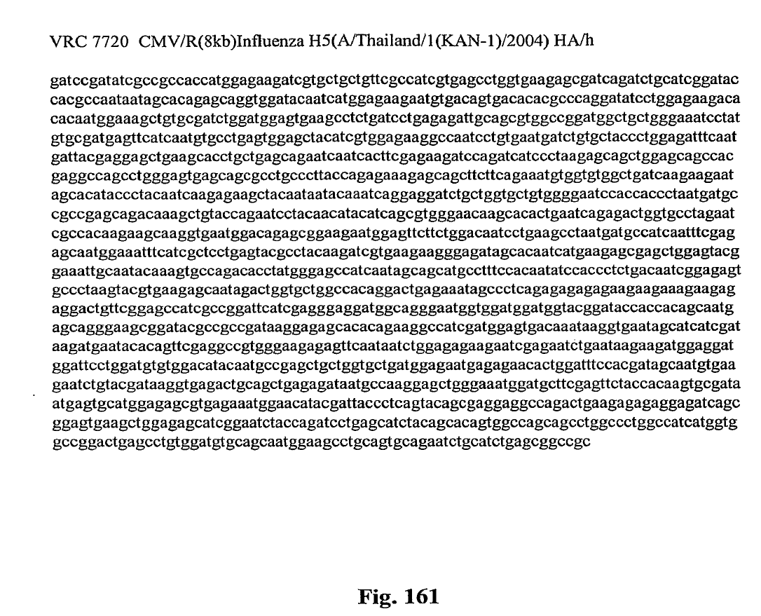

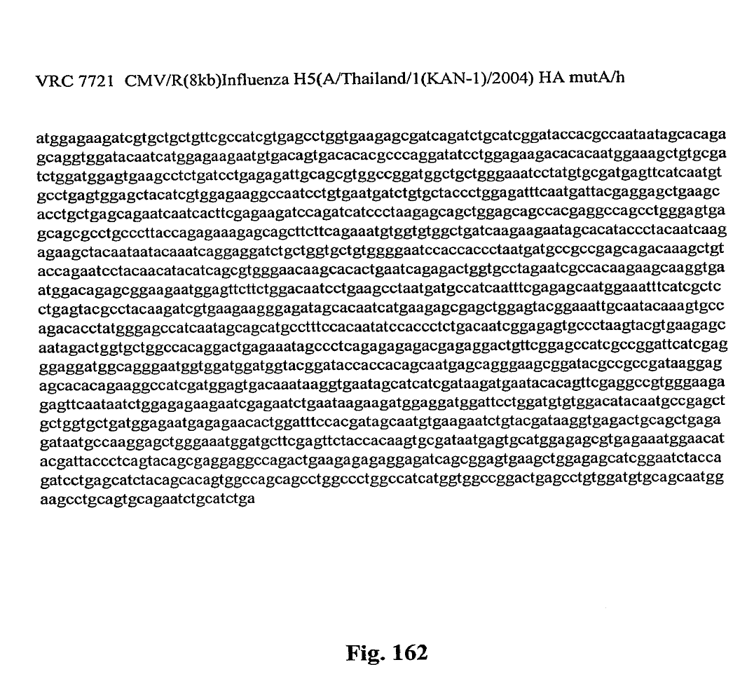

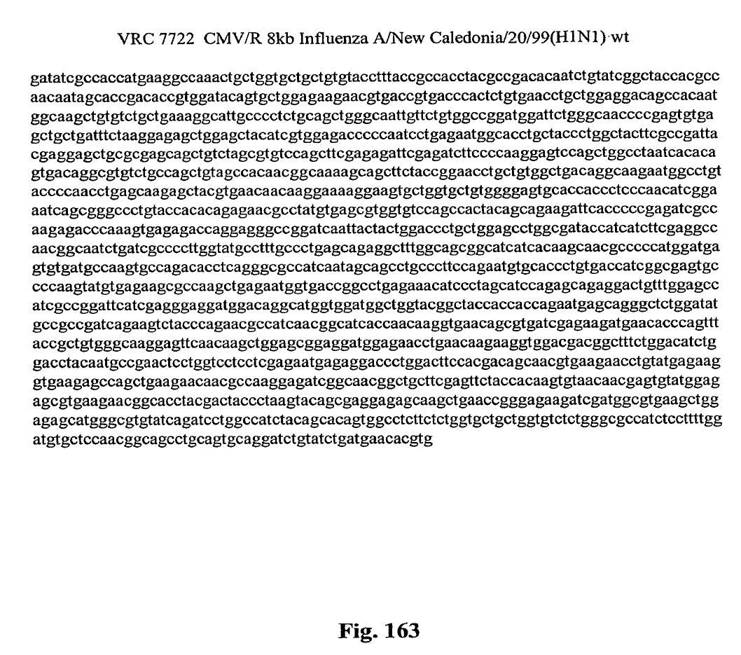

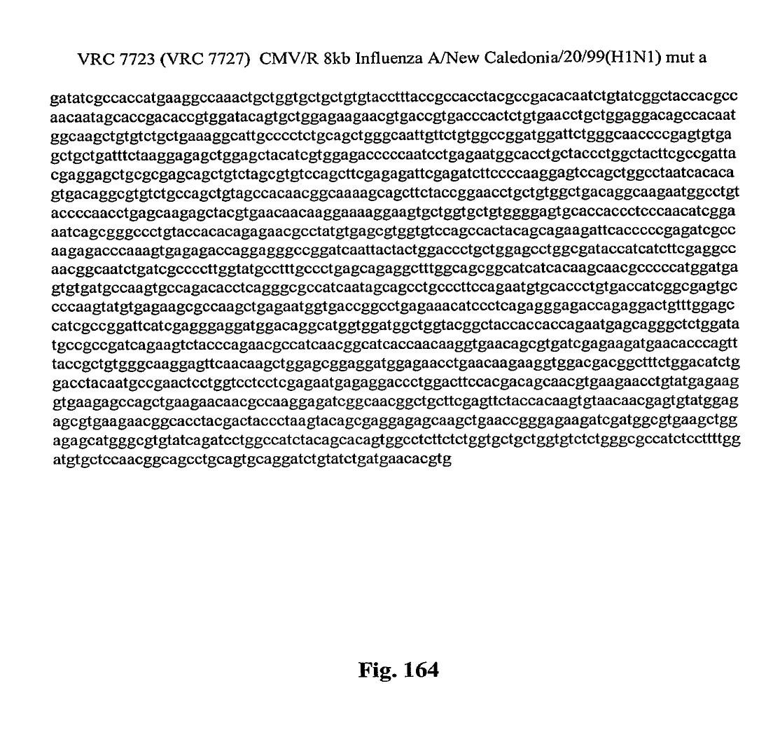

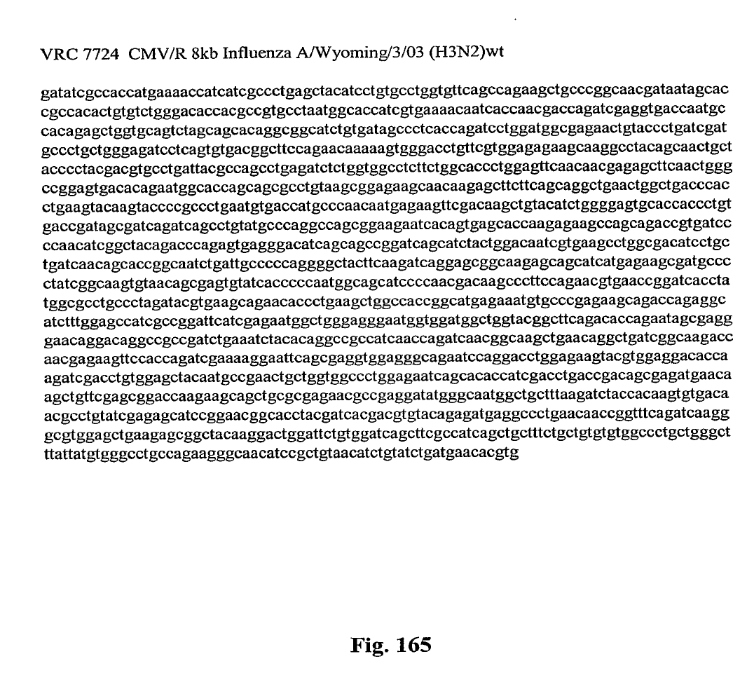

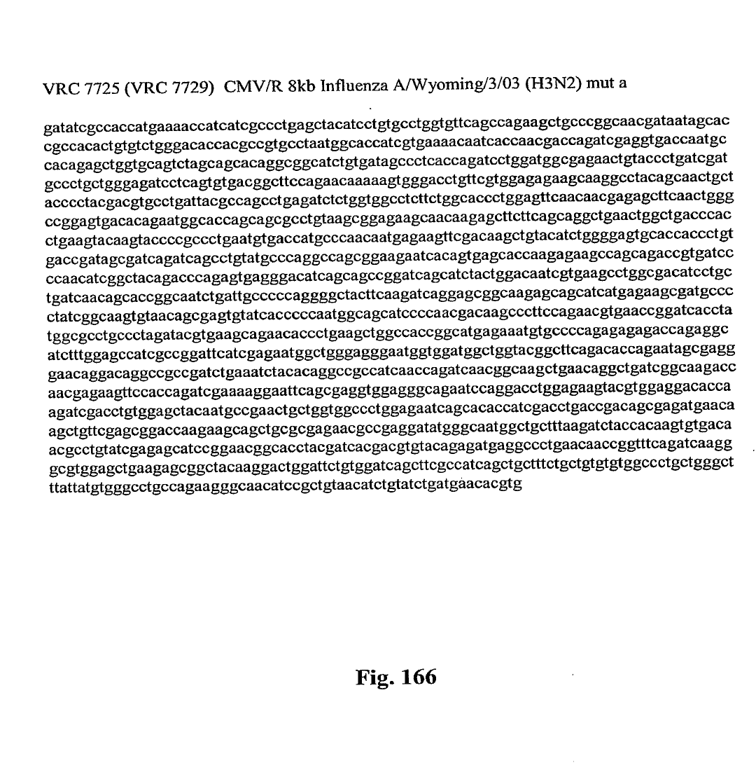

The nucleic acid sequences for six insert sequences, including VRC7720, VRC7721, VRC7722, VRC 7723 (VRC 7727), VRC 7724, and VRC 7725 (VRC 7729) are given in FIGS. 161-166.

TABLE 4
Description of VRC Influenza Plasmid DNA Vaccines

| | Plasmid | | | |
|---|---|---|---|---|
| | Number | Vaccine Product | Source of HA Gene Insert | Description of HA Gene Insert |
| Influenza A | VRC-7727 | VRC-FLUDNA031-00-VP | Influenza A New Caledonia 20/99 | Genebank #AY289929 |
| vaccines | | | (H1N1), mutant | Expresses HA w/protease |
| | | | | cleavage site modification |
| | VRC-7729 | VRC-FLUDNA033-00-VP | Influenza A Wyoming 3/03 | Genebank #AY531033 |
| | | | (H3N2), mutant | Expresses HA w/protease |
| | | | | cleavage site modification |
| Avian | VRC-7721 | VRC-AVIDNA035-00-VP | Influenza A A/Thailand/1 (Kan-1) | Genebank #AY555150 |
| Influenza | | | 2004 (H5N1), mutant | Expresses HA w/protease |
| vaccines | | | | cleavage site modification |

### Sequences of CMV/R and CMV/R 8κB Promoters and Plasniids

The CMV/R and CMV/R 8κB plasmids are 99.1% identical throughout their entire length (minus the inserted HA gene). The only areas of divergence are within the sequences of the CMV/R and CMV/R 8κB promoters shown in FIG. 167.

Apart from the modified protease cleavage sites, the amino acid sequences in all the influenza plasmids are the same as the wild-type HA proteins, but the gene sequences have been modified for optimal expression in human cells. These plasmids have been constructed in the 1012 plasmid backbone with the CMV/R 8κB promoter.

Referring to FIG. 168, the amino acid sequences of the VRC 7721 and VRC 7720 inserts are aligned, highlighting the modified protease cleavave site in VRC 7721.

### Immunogenicity Studies of CMV/R and CMV/R 8κB Plasmid DNA Vectors in Mice

Non-clinical, non-GLP immunogenicity studies were conducted by investigators at the Vaccine Research Center, National Institutes of Allergy and Infectious Diseases, National Institutes of Health, Bethesda, MD with CMV/R and CMV/R 8κB plasmid DNA vectors expressing lade B envelope in mice. HIV clade B (Ba1 strain) envelope gp145 ΔCF1ΔV12 is the same modified Env gene expressed by the CMV/R plasmid contained in the VRC HIV vaccine product VRC-HIVDNAO16-00-VP (BB-IND 11750). Several assays were used to evaluate immune responses elicited by the vaccine. Cellular immune responses, interferon gamma (IFN-γ) and tumor necrosis factor alpha (TNF-α) production by antigen stimulated cells, was measured by the flow cytometry-based intracellular cytokine staining (ICS) assay. In this system, the stimulated cells are characterized by phenotypic lymphocyte markers, allowing for precise quantification of the type of cells (for example CD4+ or CD8+ T-lymphocytes) responding to vaccine antigens. Humoral immune responses were measured using an Enzyme-Linked Immunosorbant Assay (ELISA) or a modified assay where the purified HIV envelope protein, (prepared from cells transfected with the same plasmid DNA vector), was bound to the test plate system.

Intracellular Flow Cytometric Analysis of HIV-1 Protein-specific CD4+ and CD8+ Responses after Vaccination

Harvested spleen cells (106 cells/peptide pool) were stimulated for 6 hours. The last five hours of stimulation occurred in the presence of 10 μg/mL brefeldin A (Sigina), with peptide pools having the same amino acid sequences as those expressed by the vaccine vectors. All peptides used in this report were 15-mers overlapping by 11 amino acids that spanned the complete sequence of the genes tested. Cells were permeabilized, fixed and stained with monoclonal antibodies (rat anti-mouse cell surfaces antigens CD3, CD4 and CD8, Pharmingen) followed by multiparametric flow cytometry to detect the IFN-γ and TNF-α positive cells in the CD4+ or CD8+ T-cell population.

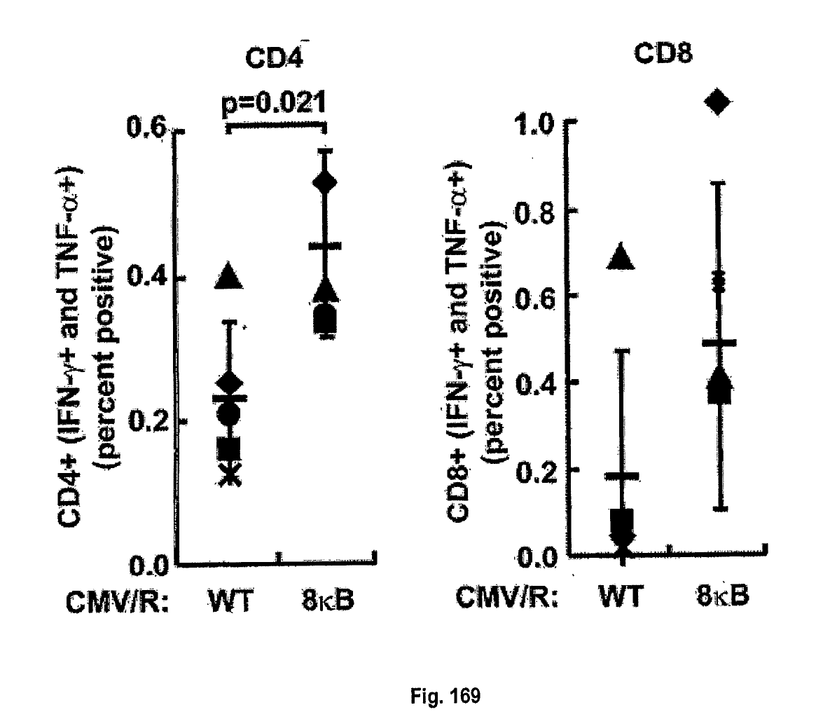

Statistical analyses of the observed CD4+ and CD8+ responses between control plasmid-vaccinated and test article-vaccinated mice were performed by the Mann-Whitney test using GraphPad Prism 3.0 software, San Diego, Calif. Assuming a frequency of >0.1% cytokine producing cells represented a positive result, then CD4+responses were observed in 5/5 of CMV/R wild-type (wt) vaccinated mice and in 5/5 of CMV/R 8κB (8κB) mice. There was a significantly higher CD4+response in 8κB vaccinated mice when compared to those vaccinated with wild-type vectors (p=0.021). CD8+ responses were observed in 2/5 of wt-vaccinated mice and in 4/5 8κB vaccinated mice as described in FIG. 169.

Referring to FIG. 169, intracellular flow cytometric analysis of gp145 env-specific CD4+and CD8+T-cell responses of immunized mice was performed. Groups of mice (5/group were vaccinated with 2.5 μg DNA plasmid, by needle-and syringe, three times (at three week intervals) and immune responses were tested 10 days after the injection. Spleens were removed aseptically, gently homogenized to a single-cell suspension, washed and re-suspended to a final concentration of 10⁶ cells/mL. Each symbol represents the percent positive cells in the CD4+ (left panel) or CD8+ (right panel) T-cell population for one animal. The mean response for the responding animals is indicated by horizontal bars. P values represent comparison of groups by Mann-Wbitney nonparametric analysis.

### ELISA Assays

Ninety-six well ELISA plates were coated with 2 μg/ml of affinity-column purified gp140 (dCFI(dV12)(Ba1) overnight at 4° C., blocked with PBS containing 5% skim milk and 2% BSA. The plates were washed with PBS+0.5% Tween-20, and incubated with 100 μL of serum from the vaccinated mice diluted in PBS+2% BSA, added in two-fold serial dilutions to each well, beginning at a dilution of 1:2400, followed by horseradish peroxidase-conjugated goat antimouse immunoglobulin 6 (IgG) and substrate (Fast o-Phenylenediamine dihydrochloride, Sigma). The reaction was stopped by the addition of 50 μL of 1N H₂SO₄, and the optical density was read at 450 nm.

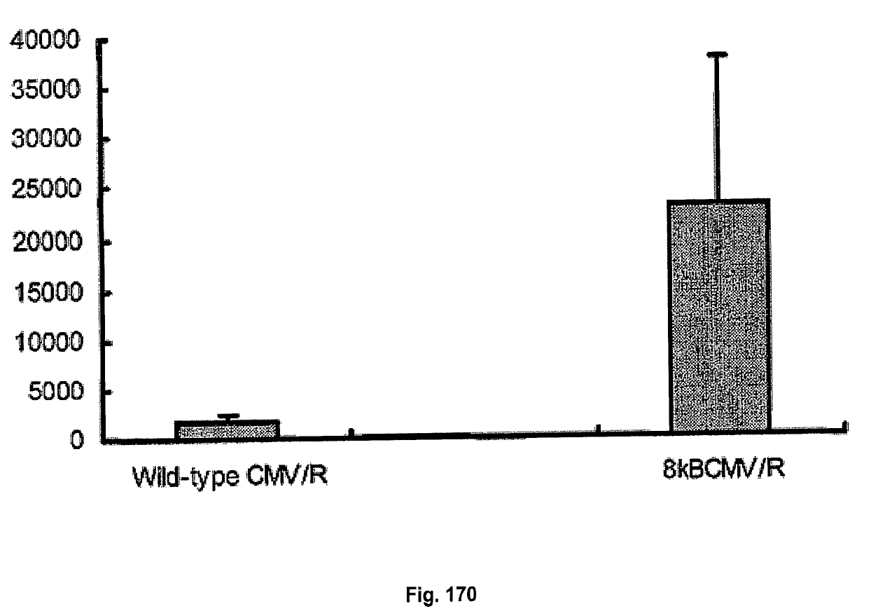

The mean antibody responses were much higher (average ELISA titer˜1:23,040) in 8κB vaccinated mice compared to wild-type CMV/R (˜1:1,680); p=0.011 as shown in FIG. 170.

Referring to FIG. 170, end-point dilutions were determined for antibody responses in mice vaccinated with wild-type CMV/R or CMV/R 8κB plasmid DNA expressing HIV gp145. Antibody responsesto HIV gp145 protein in immunized mice, measured by ELISA, is represented on the Y-axis. The thick bar on the X-axis represents the mean of ten test animals vaccinated with CMV/R or CMV/R 8κB. Error bars represent the standard deviation of the mean at each dilution.

### Potency of Influenza Plasmid DNA Vectors in Mice

Non-clinical, non-GLP immunogenicity studies in mice were conducted at the NIH Vaccine Research Center, in collaboration with the Center for Disease Control and Prevention. Mice were immunized with a CMV/R 8κB plasmid DNA vector expressing avian influenza hemagglutinin (HA) protein (influenza A/Thailand/1(KAN-1)J2004 (H5N1), followed by a lethal challenge with avian flu (influenza A/Vietnam/1203 (H5N1). After challenge, mice were monitored daily for disease signs for 21 days postinfection (p.i.). Individual body weights were recorded for each group on various days p.i.

The study demonstrated that protective immunity to lethal H5N1 challenge was conferred by vaccination with a CMV/R 8Kκ plasmid DNA vector expressing H5 hemaglutininin. All vaccinated animals survived challenge whereas all the control animals died as shown in FIG. 171. In addition, H5 plasmid DNA vaccinated animals experienced less weight loss than animals vaccinated with an empty vector control.

Referring to FIG. 171, protective immunity to lethal H5N1 Influenza challenge in mice vaccinated with a CMV/R 8κB plasmid DNA vector expressing H5 Hemagglutinin is shown. Two groups of Balb/C mice (10 mice/H5 group and 5 mice/control group) were injected bilaterally into the hind leg muscle with 5 μg DNA (100 mL) at 3 timepoints, each 21 days apart. Mice were injected either with a CMV/R 8κB plasmid DNA vector expressing H5 hemagglutinin (H5) or an empty CMV/R 8κB plasmid DNA control. Two weeks after the third and final vaccination, mice were challenged intranasally with 100 LD₅₀ of A/Vietnam/1203 (H5N1) in a volume of 50 μL.

Summary of Preclinical and Clinical Experience with Plasmid DNA Backbone Elements Used in VRC Phase I Clinical Trials

Plasmids containing the VRC-1012 backbone (Hartikka, J. et al. 1996 Hum Gen Ther 7:1205-1217) and the CMV/R promoter elements (Barouch, D. H. et al. 2005 J Virol 79:8828-8834) have undergone standard preclinical safety testing and have been evaluated in multiple human clinical trials as elements of DNA vaccines (VRC-1012, CMV/R) with demonstrated clinical safety. Preclinical and clinical testing of plasmids containing these elements is summarized in Table 5 below.

TABLE 5
Preclinical and Clinical Experience with Plasmid DNA Vaccines

| | | | Vaccine | | | | Clinical Testing | |
|---|---|---|---|---|---|---|---|---|
| | BB- | | (Number of | Preclinical Testing | | | Clinical | Number of |
| Plasmid | IND | IND Sponsor | Plasmids) | Toxicity | Biodistribution | Integration | Protocol | Subjects |
| CMV | 9782 | DAIDS/NIAID | HIV (1) | + | + | + | VRC 001 | 21 * |
| Promoter & | 10681 | DAIDS/NIAID | HIV (4) | + | + | | VRC 004 | 50 * |
| VRC-1012 | | | | | | | HVTN 052 | 180 * |
| Backbone | | | | | | | RV 156 | 15 * |
| | 11289 | DAIDS/NIAID | | | | | ACTG 5187 | 20 * |
| | 10914 | DAIDS/NIAID | HIV (4) & | ** | ** | | HVTN 044 | 70 * |
| | | | IL2/Ig | | | | | |
| | 11215 | Vical, Inc. | Anthrax (2) | + | + | + | AB01 101 | 40 |
| | 12242 | RCHSPB/NIAID | WNV (1) | + | + | | VRC 302 | 15 |
| CMV/R | 11294 | DAIDS/NIAID | Ebola (3) | + | + | | VRC 204 | 27 * |
| promoter & | 11750 | DAIDS/NIAID | HIV (6) | *** | *** | | VRC 007 | 15 |
| VRC-1012 | 11995 | RCHSPB/NIAID | SARS (1) | + | + | | VRC 301 | 10 |
| Backbone | 12326 | DAIDS/NIAID | HIV (6) | *** | *** | | VRC 008 | 40 |
| | | | | | | | HVTN 204 | 480 * |
| | | | | | | | IAVI V001 | 64 * |
| | | | | | | | RV 172 | 324 * |

* Includes control subjects
** Toxicity and biodistribution studies for the HIV (4) portion of the vaccine (Clade B gag pol nef; Clade A, B, C, env) were waived by CBER due to high degree of antigen homology with HIV (2) vaccine (Clade B gag pol nef, Clade B env) plasmids
*** Toxicity and biodistribution studies for the HIV (6) vaccine with the CMV/R promoter were waived by CBER due to high degree of antigen homology with HIV (4) vaccine plasmids.
+ Indicates that the study was completed;
Note:
Ongoing additional studies testing DNA vaccines in combination with adenovector boosts in additional INDs are not listed.

### Part 5

### Production of HA Pseudotyped Lentiviral Vectors and Measurement of Neutralizing Activity of Immune Serum

Lentiviral vectors were generated by transfiection of three plasmids into 293T cells. A lentiviral vector plasmid expressing luciferase from an internal cytomegalovirus (CMV) promoter was used as a transfer vector. The packaging plasmid pCMVΔR8.2 (encoding the HIV structural and accessory proteins) was used to express the lentiviral gene products. The influenza HA protein was expressed from a plasmid.

To measure neutralizing activity of immune serum, three plasmids—a plasmid which encodes luciferase driven ty the CMV promoter; pCMVΔR8.2, which encodes the HIV structural and accessory proteins; and a plasmid encoding the influenza HA protein—were cotransfected into 293T cells and the viral supernatant was harvested 48 h after transfection. The collected supernatants were placed on 293A cells that express the receptor for HA.

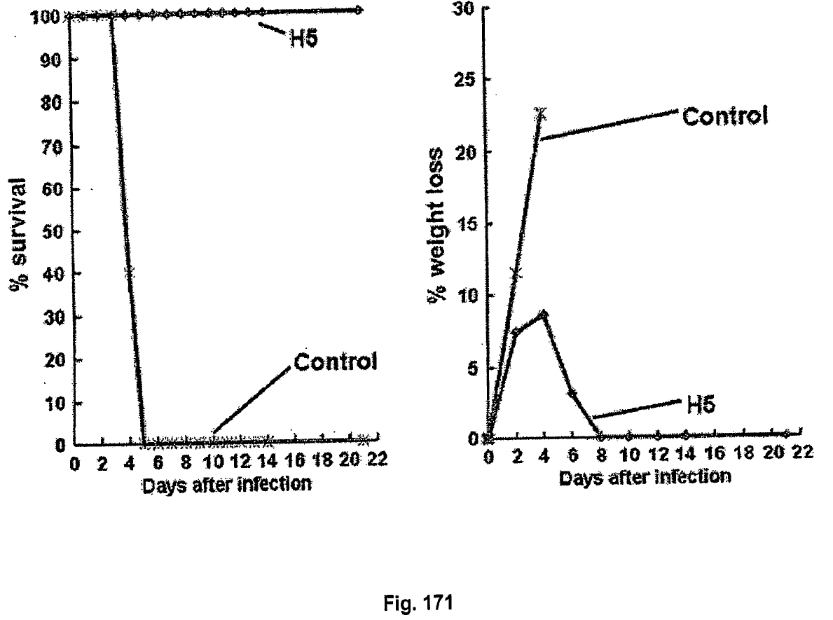

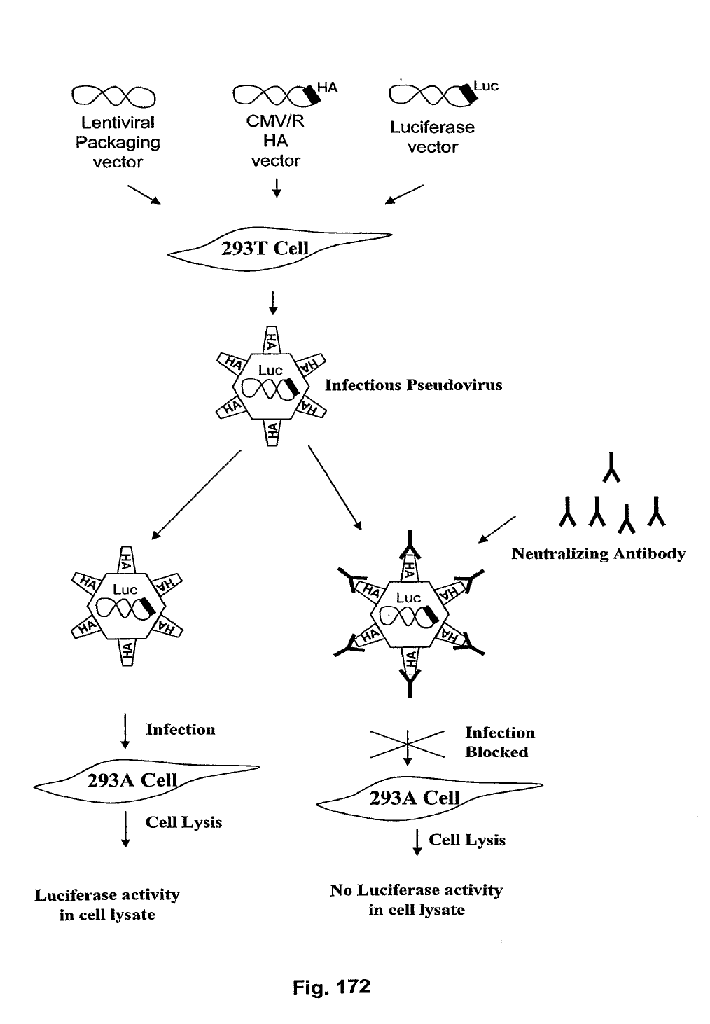

Referring to FIG. 172, influenza HA-pseudotyped lentiviral vectors expressing a luciferase reporter gene were produced as described (Yang Z-Y et al. 2004 J Virol 78:4029-4036, Naldini L et al. 1996 Science 272:263-267; and Lewis, B C et al. 2001 J Virol 75:9339-9344). Briefly, 293T cells were cotransfected by using the following plasmids: 7 μg of pCMVAR8.2, 7 μg of pHR′CMV-Luc, and 125 ng CMV/R 8κB H1(1918), H1(1918)(ΔPS), H1(1918)(ΔPS2), or H1(PR/8)(ΔPS), or H5(Kan-1). pCMVDR8.2 encodes all of the structural and accessory proteins for the lentiviral particles. Cells were transfected overnight, washed, and replenished with fresh medium. Forty-eight hours later, supernatants were harvested, filtered through a 0.45-μm syringe filter, aliquotted, and used immediately or frozen at −80° C. For neutralization assays, antisera were mixed with 100 μl of pseudoviruses at various dilutions and added to 293A cells (Invitrogen, Carlsbad, Calif.) in 48-well dishes (30,000 cells per well). Plates were washed, and fresh medium was added 14-16 h later. Forty-eight hours after infection, cells were lysed in mammalian cell lysis buffer (Promega, Madison, Wis.). A standard quantity of cell lysate was used in a luciferase assay with luciferase assay reagent (Promega) according to the manufacturer's protocol. Example neutralization assay data are given in Table 6.

TABLE 6
Immunization with plasmids

| | Viruses for neutralization Assay | | | | | |
|---|---|---|---|---|---|---|
| | VN/1203/ | Ck/KOR/ES/ | PR/ | VN/1203/ | HK/213/ | HK/491/ |
| | 2004(H5N1) | 2003(H5N1) | 8(H1N1) | 2004(H5N1) | 2003(H5N1) | 97(H5N1) |
| | Threshold dilution | | | | | |
| | 1X = 1:80 | 1X = 1:160 | 1X < 1:40 | 1X < 1:20 | 1X < 1:20 | 1X < 1:20 |
| | dilution | dilution | dilution | dilution | dilution | dilution |
| CMV/R-HA (H1) | 1X | 1X | 64X | 1X | 1X | 1X |
| CMV/R-HA-mutant A (H1) | N/D | N/D | 128X | N/D | N/D | N/D |
| CMV/R-HA-mutant B (H1) | N/D | N/D | 4X | N/D | N/D | N/D |
| CMV/R-HA (H5) | 4X | 1X | 1X | 1X | 1X | 1X |
| CMV/R-HA-mutant A (H5) | 8X | ½X | N/D | 1.4X | 3.2X | 1.4X |
| CMV/R-HA-mutant B (H5) | 2X | ½X | N/D | 1X | 1X | 1X |
| CMV/R-HA (H1) + | ½X | ½X | 64X | 1X | 1X | 1X |
| CMV/R-NA(N1) | | | | | | |
| CMV/R-HA-mutant A(H1) + | N/D | N/D | 64X | N/D | N/D | N/D |
| CMV/R-NA(N1) | | | | | | |
| CMV/R-HA-mutant B(H1) + | N/D | N/D | 32X | N/D | N/D | N/D |
| CMV/R-NA(N1) | | | | | | |
| CMV/R-HA (H5) + | 2X | ½X | 1X | 1X | 1X | 1X |
| CMV/R-NA(N1) | | | | | | |
| CMV/R-HA-mutant A(H5) + | 1X | ½X | N/D | 1X | 1X | 1X |
| CMV/R-NA(N1) | | | | | | |
| CMV/R-HA-mutant B(H5) + | 1X | ½X | N/D | 1X | 1X | 1X |
| CMV/R-NA(N1) | | | | | | |
| CMV/R + CMV/R-NA(N1) | ½X | 1X | 1X | 1X | 1X | 1X |
| CMV/R | X | 1X | N/D | 1X | 1X | 1X |

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1-170. (canceled)

171. A nucleic acid molecule comprising:
a CMV/R or CMV/R 8κB backbone; and
a polynucleotide encoding a modified hemaagglutinin (HA) protein, wherein the protein comprises a modified proteolytic cleavage site that reduces proteolytic processing of the HA protein.

172. The nucleic acid molecule of claim 171, wherein the modified HA protein comprises the amino acids PQRETR in the proteolytic cleavage site.

173. The nucleic acid molecule of claim 171, wherein the backbone is the CMV/R backbone.

174. The nucleic acid molecule of claim 171, wherein the backbone is the CMV/R 8κB backbone.

175. The nucleic acid molecule of claim 171, wherein the HA protein is encoded with a truncation at the carboxy terminal end.

176. The nucleic acid molecule of claim 171, wherein the polynucleotide is codon optimized for humans.

177. The nucleic acid molecule of claim 171, wherein said molecule is at least 95% identical to plasmid VRC 9123.

178. The nucleic acid molecule of claim 171, wherein the HA protein is an A/Thailand/1 (KAN-1)/2004 strain of HA.

179. The nucleic acid molecule of claim 178, wherein the modified HA protein comprises the amino acids PQRETR in the proteolytic cleavage site.

180. The nucleic acid molecule of claim 178, wherein the polynucleotide is codon optimized for humans.

181. The nucleic acid molecule of claim 178, wherein said molecule is at least 95% identical to plasmid VRC 7720.

182. The nucleic acid molecule of claim 171, wherein said molecule is at least 95% identical to plasmid VRC7721.

183. The nucleic acid molecule of claim 171, wherein said molecule is at least 95% identical to plasmid VRC7722.

184. The nucleic acid molecule of claim 171, wherein said molecule is at least 95% identical to plasmid VRC7727.

185. A pharmaceutical composition comprising:
a CMV/R or CMV/R 8κB backbone;
a polynucleotide encoding a modified hemaagglutinin (HA) protein, wherein the protein comprises a modified proteolytic cleavage site that reduces proteolytic processing of the HA protein; and
a pharmaceutically acceptable solution in a therapeutically effective dose.

186. The composition of claim 185, additionally comprising an adjuvant or nucleic acid encoding an adjuvant.

187. The composition of claim 186, wherein said adjuvant is a cytokine.

188. The composition of claim 185, for use as a vaccine to prevent influenza infection in a mammal.

189. A vaccine composition comprising a vector having a CMV/R or CMV/R 8κB backbone and a polynueleotide encoding a modified hemaagglutinin (HA) protein, wherein the protein comprises a modified proteolytic cleavage site that reduces proteolytic processing of the HA protein.

190. The vaccine composition of claim 189, wherein said HA protein is an A/Thailand/1 (KAN-1)/2004 strain of HA.

191. The vaccine composition of claim 189, wherein said composition comprises a plurality of vectors encoding a modified HA proteins from different serotypes of influenza viruses.

192. A pseudotyped lentiviral particle pseudotyped with an influenza HA protein comprising:
(a) a lentiviral vector plasmid expressing luciferase,
(b) lentiviral structural and accessory proteins sufficient for assembly of a lentiviral particle, and
(c) influenza HA protein, wherein the influenza HA protein effectively pseudotypes the lentiviral particle.

193. A method of preventing the symptoms of an influenza A infection, comprising:
identifying a person susceptible to influenza A infection; and
administering the nucleic acid molecule of claim 171 to the person.
